SEQUENCE LISTING (37 sequences)

SEQ ID NO: 1 - DNA, 702 bp, Homo sapiens
  source 1..702: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 2 - DNA, 1368 bp, Homo sapiens
  source 1..1368: mol_type = genomic DNA
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 3 - DNA, 281 bp, Homo sapiens
  source 1..281: mol_type = genomic DNA
attcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 4 - DNA, 939 bp, Homo sapiens
  source 1..939: mol_type = genomic DNA
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctag

SEQ ID NO: 5 - DNA, 1506 bp, Homo sapiens
  source 1..1506: mol_type = genomic DNA
atggctgcagctagcgtgaccccccctggctccctggagttgctacagcccggcttctccaagaccctcctggggaccaagctggaagccaagtacctgtgctccgcctgcagaaacgtcctccgcaggcccttccaggcgcagtgtggccaccggtactgctccttctgcctggccagcatcctcagctctgggcctcagaactgtgctgcctgtgttcacgagggcatatatgaagaaggcatttctattttagaaagcagttcggccttcccagataatgctgcccgcagggaggtggagagcctgccggccgtctgtcccagtgatggatgcacctggaaggggaccctgaaagaatacgagagctgccacgaaggccgctgcccgctcatgctgaccgaatgtcccgcgtgcaaaggcctggtccgccttggtgaaaaggagcgccacctggagcacgagtgcccggagagaagcctgagctgccggcattgccgggcaccctgctgcggagcagacgtgaaggcgcaccacgaggtctgccccaagttccccttaacttgtgacggctgcggcaagaagaagatcccccgggagaagtttcaggaccacgtcaagacttgtggcaagtgtcgagtcccttgcagattccacgccatcggctgcctcgagacggtagagggtgagaaacagcaggagcacgaggtgcagtggctgcgggagcacctggccatgctactgagctcggtgctggaggcaaagcccctcttgggagaccagagccacgcggggtcagagctcctgcagaggtgcgagagcctggagaagaagacggccacttttgagaacattgtctgcgtcctgaaccgggaggtggagagggtggccatgactgccgaggcctgcagccggcagcaccggctggaccaagacaagattgaagccctgagtagcaaggtgcagcagctggagaggagcattggcctcaaggacctggcgatggctgacttggagcagaaggtcttggagatggaggcatccacctacgatggggtcttcatctggaagatctcagacttcgccaggaagcgccaggaagctgtggctggccgcatacccgccatcttctccccagccttctacaccagcaggtacggctacaagatgtgtctgcgtatctacctgaacggcgacggcaccgggcgaggaacacacctgtccctcttctttgtggtgatgaagggcccgaatgacgccctgctgcggtggcccttcaaccagaaggtgaccttaatgctgctcgaccagaataaccgggagcacgtgattgacgccttcaggcccgacgtgacttcatcctcttttcagaggccagtcaacgacatgaacatcgcaagcggctgccccctcttctgccccgtctccaagatggaggcaaagaattcctacgtgcgggacgatgccatcttcatcaaggccattgtggacctgacagggctctaa

SEQ ID NO: 6 - DNA, 1044 bp, Homo sapiens
  source 1..1044: mol_type = genomic DNA
atggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactga

SEQ ID NO: 7 - DNA, 4124 bp, Homo sapiens
  source 1..4124: mol_type = genomic DNA
atgagcacggaggcggacgagggcatcactttctctgtgccacccttcgccccctcgggcttctgcaccatccccgagggcggcatctgcaggaggggaggagcggcggcggtgggcgagggcgaggagcaccagctgccaccgccgccgccgggcagtttctggaacgtggagagcgccgctgcccctggcatcggttgtccggcggccacctcctcgagcagtgccacccgaggccggggcagctctgttggcgggggcagccgacggaccacggtggcatatgtgatcaacgaagcgagccaagggcaactggtggtggccgagagcgaggccctgcagagcttgcgggaggcgtgcgagacagtgggcgccaccctggaaccctgcattttgggaaactcgactttggagaaaccaccgtgctggaccgcttttacaatgcagatattgcggtggtggagatgagcgatgccttccggcagccgtccttgttttaccaccttggggtgagagaaagtttcagcatggccaacaacatcatcctctactgcgatactaactcggactctctgcagtcactgaaggaaatcatttgccagaagaatactatgtgcactgggaactacacctttgttccttacatgataactccacataacaaagtctactgctgtgacagcagcttcatgaaggggttgacagagctcatgcaaccgaacttcgagctgcttcttggacccatctgcttacctcttgtggatcgttttattcaacttttgaaggtggcacaagcaagttctagccagtacttccgggaatctatactcaatgacatcaggaaagctcgtaatttatacactggtaaagaattggcagctgagttggcaagaattcggcagcgagtagataatatcgaagtcttgacagcagatattgtcataaatctgttactttcctacagagatatccaggactatgattctattgtgaagctggtagagactttagaaaaactgccaacctttgatttggcctcccatcaccatgtgaagtttcattatgcatttgcactgaataggagaaatctccctggtgacagagcaaaagctcttgatattatgattcccatggtgcaaagcgaaggacaagttgcttcagatatgtattgcctagttggtcgaatctacaaagatatgtttttggactctaatttcacggacactgaaagcagagaccatggagcttcttggttcaaaaaggcatttgaatctgagccaacactacagtcaggaattaattatgcggtcctcctcctggcagctggacaccagtttgaatcttcctttgagctccggaaagttggggtgaagctaagtagtcttcttggtaaaaagggaaacttggaaaaactccagagctactgggaagttggattttttctgggggccagcgtcctagccaatgaccacatgagagtcattcaagcatctgaaaagctttttaaactgaagacaccagcatggtacctcaagtctattgtagagacaattttgatatataagcattttgtgaaactgaccacagaacagcctgtggccaagcaagaacttgtggacttttggatggatttcctggtcgaggccacaaagacagatgttactgtggttaggtttccagtattaatattagaaccaaccaaaatctatcaaccttcttatttgtctatcaacaatgaagttgaggaaaagacaatctctatttggcacgtgcttcctgatgacaagaaaggtatacatgagtggaattttagtgcctcttctgtcaggggagtgagtatttctaaatttgaagaaagatgctgctttctttatgtgcttcacaattctgatgatttccaaatctatttctgtacagaacttcattgtaaaaagttttttgagatggtgaacaccattaccgaagagaaggggagaagcacagaggaaggagactgtgaaagtgacttgctggagtatgactatgaatatgatgaaaatggtgacagagtcgttttaggaaaaggcacttatgggatagtctacgcaggtcgggacttgagcaaccaagtcagaattgctattaaggaaatcccagagagagacagcagatactctcagcccctgcatgaagaaatagcattgcataaacacctgaagcacaaaaatattgtccagtatctgggctctttcagtgagaatggtttcattaaaatcttcatggagcaggtccctggaggaagtctttctgctctccttcgttccaaatggggtccattaaaagacaatgagcaaacaattggcttttatacaaagcaaatactggaaggattaaaatatctccatgacaatcagatagttcaccgggacataaagggtgacaatgtgttgattaatacctacagtggtgttctcaagatctctgacttcggaacatcaaagaggcttgctggcataaacccctgtactgaaacttttactggtaccctccagtatatggcaccagaaataatagataaaggaccaagaggctacggaaaagcagcagacatctggtctctgggctgtacaatcattgaaatggccacaggaaaacccccattttatgaactgggagaaccacaagcagctatgttcaaggtgggaatgtttaaagtccaccctgagatcccagagtccatgtctgcagaggccaaggcattcatactgaaatgttttgaaccagatcctgacaagagagcctgtgctaacgacttgcttgttgatgagtttttaaaagtttcaagcaaaaagaaaaagacacaacctaagctttcagctctttcagctggatcaaatgaatatctcaggagtatatccttgccggtacctgtgctggtggaggacaccagcagcagcagtgagtacggctcagtttcacccgacacggagttgaaagtggaccccttctctttcaaaacaagagccaagtcctgcggagaaagagatgtcaagggaattcggacactctttttgggcattccagatgagaattttgaagatcacagtgctcctccttcccctgaagaaaaagattctggattcttcatgctgaggaaggacagtgagaggcgagctacccttcacaggatcctgacggaagaccaagacaaaattgtgagaaacctaatggaatctttagctcagggggctgaagaaccgaaactaaaatgggaacacatcacaaccctcattgcaagcctcagagaatttgtgagatccactgaccgaaaaatcatagccaccacactgtcaaagctgaaactggagctggacttcgacagccatggcattagccaagtccaggtggtactctttggttttcaagatgctgtcaataaagttcttcggaatcataacatcaagccgcactggatgtttgccttagacagtatcattcggaaggcggtacagacagccattaccatcctggttccagaactaaggccacatttcagccttgcatctgagagtgatactgctgatcaagaagacttggatgtagaagatgaccatgaggaacagccttcaaatcaaactgtccgaagacctcaggctgtcattgaagatgctgtggctacctcaggcgtgagcacgctcagttctactgtgtctcatgattcccagagtgctcaccggtcactgaatgtacagcttggaaggatgaaaatagaaaccaatagattactggaagaattggttcggaaagagaaagaattacaagcactccttcatcgagctattgaagaaaaagaccaagaaattaaacacctgaagcttaagtcccaacccatagaaattcctgaattgcctgtatttcatctaaattcttctggcacaaatactgaagattctgaacttaccgactggctgagagtgaatggagctgatgaagacactataagccggtttttggctgaagattatacactattggatgttctctactatgttacacgtgatgacttaaaatgcttgagactaaggggagggatgctgtgcacactgtggaaggctatcattgactttcgaaacaaacagacttga

SEQ ID NO: 8 - DNA, 1128 bp, Homo sapiens
  source 1..1128: mol_type = genomic DNA
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 9 - DNA, 1140 bp, Homo sapiens
  source 1..1140: mol_type = genomic DNA
atggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctag

SEQ ID NO: 10 - DNA, 1083 bp, Homo sapiens
  source 1..1083: mol_type = genomic DNA
atggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaa

SEQ ID NO: 11 - DNA, 2265 bp, Homo sapiens
  source 1..2265: mol_type = genomic DNA
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 12 - DNA, 954 bp, Homo sapiens
  source 1..954: mol_type = genomic DNA
atgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatga

SEQ ID NO: 13 - DNA, 2139 bp, Homo sapiens
  source 1..2139: mol_type = genomic DNA
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 14 - DNA, 1284 bp, Homo sapiens
  source 1..1284: mol_type = genomic DNA
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatga

SEQ ID NO: 15 - DNA, 1446 bp, Homo sapiens
  source 1..1446: mol_type = genomic DNA
atgggggccttggccagagccctgccgtccatactgctggcattgctgcttacgtccaccccagaggctctgggtgccaaccccggcttggtcgccaggatcaccgacaagggactgcagtatgcggcccaggaggggctattagctctgcagagtgagctgctcaggatcacgctgcctgacttcaccggggacttgaggatcccccacgtcggccgtgggcgctatgagttccacagcctgaacatccacagctgtgagctgcttcactctgcgctgaggcctgtccctggccagggcctgagtctcagcatctccgactcctccatccgggtccagggcaggtggaaggtgcgcaagtcattcttcaaactacagggctcctttgatgtcagtgtcaagggcatcagcatttcggtcaacctcctgttgggcagcgagtcctccgggaggcccacagttactgcctccagctgcagcagtgacatcgctgacgtggaggtggacatgtcgggagacttggggtggctgttgaacctcttccacaaccagattgagtccaagttccagaaagtactggagagcaggatttgcgaaatgatccagaaatcggtgtcctccgatctacagccttatctccaaactctgccagttacaacagagattgacagtttcgccgacattgattatagcttagtggaagcccctcgggcaacagcccagatgctggaggtgatgtttaagggtgaaatctttcatcgtaaccaccgttctccagttaccctccttgctgcagtcatgagccttcctgaggaacacaacaaaatggtctactttgccatctcggattatgtcttcaacacggccagcctggtttatcatgaggaaggatatctgaacttctccatcacagatgacatgataccgcctgactctaatatccgactgaccaccaagtccttccgacccttcgtcccacggttagccaggctctaccccaacatgaacctggaactccagggatcagtgccctctgctccgctcctgaacttcagccctgggaatctgtctgtggacccctatatggagatagatgcctttgtgctcctgcccagctccagcaaggagcctgtcttccggctcagtgtggccactaatgtgtccgccaccttgaccttcaataccagcaagatcactgggttcctgaagccaggaaaggtaaaagtggaactgaaagaatccaaagttggactattcaatgcagagctgttggaagcgctcctcaactattacatccttaacaccctctaccccaagttcaatgataagttggccgaaggcttcccccttcctctgctgaagcgtgttcagctctacgaccttgggctgcagatccataaggacttcctgttcttgggtgccaatgtccaatacatgagagtttga

SEQ ID NO: 16 - DNA, 1182 bp, Homo sapiens
  source 1..1182: mol_type = genomic DNA
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 17 - DNA, 1203 bp, Homo sapiens
  source 1..1203: mol_type = genomic DNA
atgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtga

SEQ ID NO: 18 - DNA, 957 bp, Homo sapiens
  source 1..957: mol_type = genomic DNA
atgtccaagccacccgcacccaaccccacacccccccggaacctggactcccggaccttcatcaccattggagacagaaactttgaggtggaggctgatgacttggtgaccatctcagaactgggccgtggagcctatggggtggtagagaaggtgcggcacgcccagagcggcaccatcatggccgtgaagcggatccgggccaccgtgaactcacaggagcagaagcggctgctcatggacctggacatcaacatgcgcacggtcgactgtttctacactgtcaccttctacggggcactattcagagagggagacgtgtggatctgcatggagctcatggacacatccttggacaagttctaccggaaggtgctggataaaaacatgacaattccagaggacatccttggggagattgctgtgtctatcgtgcgggccctggagcatctgcacagcaagctgtcggtgatccacagagatgtgaagccctccaatgtccttatcaacaaggagggccatgtgaagatgtgtgactttggcatcagtggctacttggtggactctgtggccaagacgatggatgccggctgcaagccctacatggcccctgagaggatcaacccagagctgaaccagaagggctacaatgtcaagtccgacgtctggagcctgggcatcaccatgattgagatggccatcctgcggttcccttacgagtcctgggggaccccgttccagcagctgaagcaggtggtggaggagccgtccccccagctcccagccgaccgtttctcccccgagtttgtggacttcactgctcagtgcctgaggaagaaccccgcagagcgtatgagctacctggagctgatggagcaccccttcttcaccttgcacaaaaccaagaagacggacattgctgccttcgtgaaggagatcctgggagaagactcatag

SEQ ID NO: 19 - DNA, 1005 bp, Homo sapiens
  source 1..1005: mol_type = genomic DNA
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaa

SEQ ID NO: 20 - DNA, 4539 bp, Homo sapiens
  source 1..4539: mol_type = genomic DNA
atggcggcggcggcggggaatcgcgcctcgtcgtcgggattcccgggcgccagggctacgagccctgaggcaggcggcggcggaggagccctcaaggcgagcagcgcgcccgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgcgagccgcggcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacgtctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagttccactcacttcaccaggatgcgtcgccgtttgatggctattgcagatgaggtggaaattgccgaagccatccagttgggcgtagaagacactttggatggtcaacaggacagcttcttgcaggcatctgttcccaacaactatctggaaaccacagagaacagttcccctgagtgcacagtccatttagagaaaactggaaaaggattatgtgctacaaaattgagtgccagttcagaggacatttctgagagactggccagcatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctagacctaccccaggtaatacaagtaaacagggagatccctcaaaaaatagcatgacacttgatctgaacagtagttccaaatgtgatgacagctttggctgtagcagcaatagtagtaatgctgttatacccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggatgccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtag

SEQ ID NO: 21 - DNA, 1974 bp, Homo sapiens
  source 1..1974: mol_type = genomic DNA
atggacgaacaggaggcattgaactcaatcatgaacgatctggtggccctccagatgaaccgacgtcaccggatgcctggatatgagaccatgaagaacaaagacacaggtcactcaaataggcagaaaaaacacaacagcagcagctcagcccttctgaacagccccacagtaacaacaagctcatgtgcaggggccagtgagaaaaagaaatttttgagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactga

SEQ ID NO: 22 - DNA, 4826 bp, Homo sapiens
  source 1..4826: mol_type = genomic DNA
atgagagaagccgctgccgcgctggtccctcctcccgcctttgccgtcacgcctgccgccgccatggaggagccgccgccaccgccgccgccgccaccaccgccaccggaacccgagaccgagtcagaacccgagtgctgcttggcggcgaggcaagagggcacattgggagattcagcttgcaagagtcctgaatctgatctagaagacttctccgatgaaacaaatacagagaatctttatggtacctctccccccagcacacctcgacagatgaaacgcatgtcaaccaaacatcagaggaataatgtggggaggccagccagtcggtctaatttgaaagaaaaaatgaatgcaccaaatcagcctccacataaagacactggaaaaacagtggagaatgtggaagaatacagctataagcaggagaaaaagatccgagcagctcttagaacaacagagcgtgatcataaaaaaaatgtacagtgctcattcatgttagactcagtgggtggatctttgccaaaaaaatcaattccagatgtggatctcaataagccttacctcagccttggctgtagcaatgctaagcttccagtatctgtgcccatgcctatagccagacctgcacgccagacttctaggactgactgtccagcagatcgtttaaagttttttgaaactttacgacttttgctaaagcttacctcagtctcaaagaaaaaagacagggagcaaagaggacaagaaaatacgtctggtttctggcttaaccgatctaacgaactgatctggttagagctacaagcctggcatgcaggacggacaattaacgaccaggacttctttttatatacagcccgtcaagccatcccagatattattaatgaaatccttactttcaaagtcgactatgggagcttcgcctttgttagagatagagctggttttaatggtacttcagtagaagggcagtgcaaagccactcctggaacaaagattgtaggttactcaacacatcatgagcatctccaacgccagagggtctcatttgagcaggtaaaacggataatggagctgctagagtacatagaagcactttatccatcattgcaggctcttcagaaggactatgaaaaatatgctgcaaaagacttccaggacagggtgcaggcactctgtttgtggttaaacatcacaaaagacttaaatcagaaattaaggattatgggcactgttttgggcatcaagaatttatcagacattggctggccagtgtttgaaatcccttcccctcgaccatccaaaggtaatgagccggagtatgagggtgatgacacagaaggagaattaaaggagttggaaagtagtacggatgagagtgaagaagaacaaatctctgatcctagggtaccggaaatcagacagcccatagataacagcttcgacatccagtcgcgggactgcatatccaagaagcttgagaggctcgaatctgaggatgattctcttggctggggagcaccagactggagcacagaagcaggctttagtagacattgtctgacttctatttatagaccatttgtagacaaagcactgaagcagatggggttaagaaagttaattttaagacttcacaagctaatggatggttccttgcaaagggcacgtatagcattggtaaagaacgatcgtccagtggagttttctgaatttccagatcccatgtggggttcagattatgtgcagttgtcaaggacaccaccttcatctgaggagaaatgcagtgctgtgtcgtgggaggagctgaaggccatggatttaccttcattcgaacctgccttcctagttctctgccgagtccttctgaatgtcatacatgagtgtctgaagttaagattggagcagagacctgctggagaaccatctctcttgagtattaagcagctggtgagagagtgtaaggaggtcctgaagggcggcctgctgatgaagcagtactaccagttcatgctgcaggaggttctggaggacttggagaagcccgactgcaacattgacgcttttgaagaggatctacataaaatgcttatggtgtattttgattacatgagaagctggatccaaatgctacagcaattacctcaagcatcgcatagtttaaaaaatctgttagaagaagaatggaatttcaccaaagaaataactcattacatacggggaggagaagcacaggccgggaagcttttctgtgacattgcaggaatgctgctgaaatctacaggaagttttttagaatttggcttacaggagagctgtgctgaattttggactagtgcggatgacagcagtgcttccgacgaaatcaggaggtctgttatagagatcagtcgagccctgaaggagctcttccatgaagccagagaaagggcttccaaagcacttggatttgctaaaatgttgagaaaggacctggaaatagcagcagaattcaggctttcagccccagttagagacctcctggatgttctgaaatcaaaacagtatgtcaaggtgcaaattcctgggttagaaaacttgcaaatgtttgttccagacactcttgctgaggagaagagtattattttgcagttactcaatgcagctgcaggaaaggactgttcaaaagattcagatgacgtactcatcgatgcctatctgcttctgaccaagcacggtgatcgagcccgtgattcagaggacagctggggcacctgggaggcacagcctgtcaaagtcgtgcctcaggtggagactgttgacaccctgagaagcatgcaggtggataatcttttactagttgtcatgcagtctgcgcatctcacaattcagagaaaagctttccagcagtccattgagggacttatgactctgtgccaggagcagacatccagtcagccggtcatcgccaaagctttgcagcagctgaagaatgatgcattggagctatgcaacaggataagcaatgccattgaccgcgtggaccacatgttcacatcagaatttgatgctgaggttgatgaatctgaatctgtcaccttgcaacagtactaccgagaagcaatgattcaggggtacaattttggatttgagtatcataaagaagttgttcgtttgatgtctggggagtttagacagaagataggagacaaatatataagctttgcccggaagtggatgaattatgtcctgactaaatgtgagagtggtagaggtacaagacccaggtgggcgactcaaggatttgattttctacaagcaattgaacctgcctttatttcagctttaccagaagatgacttcttgagtttacaagccttgatgaatgaatgcattggccatgtcataggaaaaccacacagtcctgttacaggtttgtaccttgccattcatcggaacagcccccgtcctatgaaggtacctcgatgccatagtgaccctcctaacccacacctcattatccccactccagagggattcagcactcggagcatgccttccgacgcgcggagccatggcagccctgctgctgctgctgctgctgctgctgctgctgttgctgccagtcggcccagcccctctggtggtgactctgtgctgcccaaatccatcagcagtgcccatgataccaggggttccagcgttcctgaaaatgatcgattggcttccatagctgctgaattgcagtttaggtccctgagtcgtcactcaagccccacggaggagcgagatgaaccagcatatccaagaggagattcaagtgggtccacaagaagaagttgggaacttcggacactaatcagccagagtaaagatactgcttctaaactaggacccatagaagctatccagaagtcagtccgattgtttgaagaaaagaggtaccgagaaatgaggagaaagaatatcattggtcaagtttgtgatacgcctaagtcctatgataatgttatgcacgttggcttgaggaaggtgaccttcaaatggcaaagaggaaacaaaattggagaaggccagtatgggaaggtgtacacctgcatcagcgtcgacaccggggagctgatggccatgaaagagattcgatttcaacctaatgaccataagactatcaaggaaactgcagacgaattgaaaatattcgaaggcatcaaacaccccaatctggttcggtattttggtgtggagctccatagagaagaaatgtacatcttcatggagtactgcgatgaggggactttagaagaggtgtcaaggctgggacttcaggaacatgtgattaggctgtattcaaagcagatcaccattgcgatcaacgtcctccatgagcatggcatagtccaccgtgacattaaaggtgccaatatcttccttacctcatctggattaatcaaactgggagattttggatgttcagtaaagctcaaaaacaatgcccagaccatgcctggtgaagtgaacagcaccctggggacagcagcatacatggcacctgaagtcatcactcgtgccaaaggagagggccatgggcgtgcggccgacatctggagtctggggtgtgttgtcatagagatggtgactggcaagaggccttggcatgagtatgagcacaactttcaaattatgtataaagtggggatgggacataagccaccaatccctgaaagattaagccctgaaggaaaggacttcctttctcactgccttgagagtgacccaaagatgagatggaccgccagccagctcctcgaccattcgtttgtcaaggtttgcacagatgaagaatg

SEQ ID NO: 23 - DNA, 3867 bp, Homo sapiens
  source 1..3867: mol_type = genomic DNA
atggcggggccgtgtccccggtccggggcggagcgcgccggcagctgctggcaggacccgctggccgtggcgctgagccggggccggcagctcgcggcgcccccgggccggggctgcgcgcggagccggccgctcagcgtggtctacgtgctgacccgggagccgcagcccgggctcgagcctcgggagggaaccgaggcggagccgctgcccctgcgctgcctgcgcgaggcttgcgcgcaggtcccccggccgcggccgcccccgcagctgcgcagcctgcccttcgggacgctggagctaggcgacaccgcggctctggatgccttctacaacgcggatgtggtggtgctggaggtgagcagctcgctggtacagccctccctgttctaccaccttggtgtgcgtgagagcttcagcatgaccaacaatgtgctcctctgctcccaggccgacctccctgacctgcaggccctgcgggaggatgttttccagaagaactcggattgcgttggcagctacacactgatcccctatgtggtgacggccactggtcgggtgctgtgtggtgatgcaggccttctgcggggcctggctgatgggctggtacaggctggagtggggaccgaggccctgctcactcccctggtgggccggcttgcccgcctgctggaggccacacccacagactcttgtggctatttccgggagaccattcggcgggacatccggcaggcgcgggagcggttcagtgggccacagctgcggcaggagctggctcgcctgcagcggagactggacagcgtggagctgctgagccccgacatcatcatgaacttgctgctctcctaccgcgatgtgcaggactactcggccatcattgagctggtggagacgctgcaggccttgcccacctgtgatgtggccgagcagcataatgtctgcttccactacacttttgccctcaaccggaggaacaggcctggggaccgggcgaaggccctgtctgtgctgctgccgctggtacagcttgagggctctgtggcgcccgatctgtactgcatgtgtggccgtatctacaaggacatgttcttcagctcgggtttccaggatgctgggcaccgggagcaggcctatcactggtatcgcaaggcttttgacgtagagcccagccttcactcaggcatcaatgcagctgtgctcctcattgctgccgggcagcactttgaggattccaaagagctccggctaataggcatgaagctgggctgcctgctggcccgcaaaggctgcgtggagaagatgcagtattactgggatgtgggtttctacctgggagcccagatcctcgccaatgaccccacccaggtggtgctggctgcagagcagctgtataagctcaatgcccccatatggtacctggtgtccgtgatggagaccttcctgctctaccagcacttcaggcccacgccagagccccctggagggccaccacgccgtgcccacttctggctccacttcttgctacagtcctgccaaccattcaagacagcctgtgcccagggcgaccagtgcttggtgctggtcctggagatgaacaaggtgctgctgcctgcaaagctcgaggttcggggtactgacccagtaagcacagtgaccctgagcctgctggagcctgagacccaggacattccctccagctggaccttcccagtcgcctccatatgcggagtcagcgcctcaaagcgcgacgagcgctgctgcttcctctatgcactccccccggctcaggacgtccagctgtgcttccccagcgtagggcactgccagtggttctgcggcctgatccaggcctgggtgacgaacccggattccacggcgcccgcggaggaggcggagggcgcgggggagatgttggagtttgattatgagtacacggagacgggcgagcggctggtgctgggcaagggcacgtatggggtggtgtacgcgggccgcgatcgccacacgagggtgcgcatcgccatcaaggagatcccggagcgggacagcaggttctctcagcccctgcatgaagagatcgctcttcacagacgcctgcgccacaagaacatagtgcgctatctgggctcagctagccagggcggctaccttaagatcttcatggaggaagtgcctggaggcagcctgtcctccttgctgcggtcggtgtggggacccctgaaggacaacgagagcaccatcagtttctacacccgccagatcctgcagggacttggctacttgcacgacaaccacatcgtgcacagggacataaaaggggacaatgtgctgatcaacaccttcagtgggctgctcaagatttctgacttcggcacctccaagcggctggcaggcatcacaccttgcactgagaccttcacaggaactctgcagtatatggccccagaaatcattgaccagggcccacgcgggtatgggaaagcagctgacatctggtcactgggctgcactgtcattgagatggccacaggtcgcccccccttccacgagctcgggagcccacaggctgccatgtttcaggtgggtatgtacaaggtccatccgccaatgcccagctctctgtcggccgaggcccaagcctttctcctccgaacttttgagccagacccccgcctccgagccagcgcccagacactgctgggggaccccttcctgcagcctgggaaaaggagccgcagccccagctccccacgacatgctccacggccctcagatgccccttctgccagtcccactccttcagccaactcaaccacccagtctcagacattcccgtgccctcaggcaccctctcagcacccacccagccccccgaagcgctgcctcagttatgggggcaccagccagctccgggtgcccgaggagcctgcggccgaggagcctgcgtctccggaggagagttcggggctgagcctgctgcaccaggagagcaagcgtcgggccatgctggccgcagtattggagcaggagctgccagcgctggcggagaatctgcaccaggagcagaagcaagagcagggggcccgtctgggcagaaaccatgtggaagagctgctgcgctgcctcggggcacacatccacactcccaaccgccggcagctcgcccaggagctgcgggcgctgcaaggacggctgagggcccagggccttgggcctgcgcttctgcacagaccgctgtttgccttcccggatgcggtgaagcagatcctccgcaagcgccagatccgtccacactggatgttcgttctggactcactgctcagccgtgctgtgcgggcagccctgggtgtgctaggaccggaggtggagaaggaggcggtctcaccgaggtcagaggagctgagtaatgaaggggactcccagcagagcccaggccagcagagcccgcttccggtggagcccgagcagggccccgctcctctgatggtgcagctgagcctcttgagggcagagactgatcggctgcgcgaaatcctggcggggaaggaacgggagtaccaggccctggtgcagcgggctctacagcggctgaatgaggaagcccggacctatgtcctggccccagagcctccaactgctctttcaacggaccagggcctggtgcagtggctacaggaactgaatgtggattcaggcaccatccaaatgctgttgaaccatagcttcaccctccacactctgctcacctatgccactcgagatgacctcatctacacccgcatcaggggagggatggtatgccgcatctggagggccatcttggcacagcgagcaggatccacaccagtcacctctggaccctga

SEQ ID NO: 24 - DNA, 1740 bp, Homo sapiens
  source 1..1740: mol_type = genomic DNA
atgtctacagcctctgccgcctcctcctcctcctcgtcttcggccggtgagatgatcgaagccccttcccaggtcctcaactttgaagagatcgactacaaggagatcgaggtggaagaggttgttggaagaggagcctttggagttgtttgcaaagctaagtggagagcaaaagatgttgctattaaacaaatagaaagtgaatctgagaggaaagcgtttattgtagagcttcggcagttatcccgtgtgaaccatcctaatattgtaaagctttatggagcctgcttgaatccagtgtgtcttgtgatggaatatgctgaagggggctctttatataatgtgctgcatggtgctgaaccattgccatattatactgctgcccacgcaatgagttggtgtttacagtgttcccaaggagtggcttatcttcacagcatgcaacccaaagcgctaattcacagggacctgaaaccaccaaacttactgctggttgcaggggggacagttctaaaaatttgtgattttggtacagcctgtgacattcagacacacatgaccaataacaaggggagtgctgcttggatggcacctgaagtttttgaaggtagtaattacagtgaaaaatgtgacgtcttcagctggggtattattctttgggaagtgataacgcgtcggaaaccctttgatgagattggtggcccagctttccgaatcatgtgggctgttcataatggtactcgaccaccactgataaaaaatttacctaagcccattgagagcctgatgactcgttgttggtctaaagatccttcccagcgcccttcaatggaggaaattgtgaaaataatgactcacttgatgcggtactttccaggagcagatgagccattacagtatccttgtcagtattcagatgaaggacagagcaactctgccaccagtacaggctcattcatggacattgcttctacaaatacgagtaacaaaagtgacactaatatggagcaagttcctgccacaaatgatactattaagcgcttagaatcaaaattgttgaaaaatcaggcaaagcaacagagtgaatctggacgtttaagcttgggagcctcccgtgggagcagtgtggagagcttgcccccaacctctgagggcaagaggatgagtgctgacatgtctgaaatagaagctaggatcgccgcaaccacaggcaacggacagccaagacgtagatccatccaagacttgactgtaactggaacagaacctggtcaggtgagcagtaggtcatccagtcccagtgtcagaatgattactacctcaggaccaacctcagaaaagccaactcgaagtcatccatggacccctgatgattccacagataccaatggatcagataactccatcccaatggcttatcttacactggatcaccaactacagcctctagcaccgtgcccaaactccaaagaatctatggcagtgtttgaacagcattgtaaaatggcacaagaatatatgaaagttcaaacagaaattgcattgttattacagagaaagcaagaactagttgcagaactggaccaggatgaaaaggaccagcaaaatacatctcgcctggtacaggaacataaaaagcttttagatgaaaacaaaagcctttctacttactaccagcaatgcaaaaaacaactagaggtcatcagaagtcagcagcagaaacgacaaggcacttcatga

SEQ ID NO: 25 - DNA, 1113 bp, Homo sapiens
  source 1..1113: mol_type = genomic DNA
atgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtga

SEQ ID NO: 26 - DNA, 954 bp, Homo sapiens
  source 1..954: mol_type = genomic DNA
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 27 - DNA, 2907 bp, Homo sapiens
  source 1..2907: mol_type = genomic DNA
atggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 28 - DNA, 2844 bp, Homo sapiens
  source 1..2844: mol_type = genomic DNA
atggcagtgatggaaatggcctgcccaggtgcccctggctcagcagtggggcagcagaaggaactccccaaagccaaggagaagacgccgccactggggaagaaacagagctccgtctacaagcttgaggccgtggagaagagccctgtgttctgcggaaagtgggagatcctgaatgacgtgattaccaagggcacagccaaggaaggctccgaggcagggccagctgccatctctatcatcgcccaggctgagtgtgagaatagccaagagttcagccccaccttttcagaacgcattttcatcgctgggtccaaacagtacagccagtccgagagtcttgatcagatccccaacaatgtggcccatgctacagagggcaaaatggcccgtgtgtgttggaagggaaagcgtcgcagcaaagcccggaagaaacggaagaagaagagctcaaagtccctggctcatgcaggagtggccttggccaaacccctccccaggacccctgagcaggagagctgcaccatcccagtgcaggaggatgagtctccactcggcgccccatatgttagaaacaccccgcagttcaccaagcctctgaaggaaccaggccttgggcaactctgttttaagcagcttggcgagggcctacggccggctctgcctcgatcagaactccacaaactgatcagccccttgcaatgtctgaaccacgtgtggaaactgcaccacccccaggacggaggccccctgcccctgcccacgcaccccttcccctatagcagactgcctcatcccttcccattccaccctctccagccctggaaacctcaccctctggagtccttcctgggcaaactggcctgtgtagacagccagaaacccttgcctgacccacacctgagcaaactggcctgtgtagacagtccaaagcccctgcctggcccacacctggagcccagctgcctgtctcgtggtgcccatgagaagttttctgtggaggaatacctagtgcatgctctgcaaggcagcgtgagctcaggccaggcccacagcctgaccagcctggccaagacctgggcagcaaggggctccagatcccgggagcccagccccaaaactgaggacaacgagggtgtcctgctcactgagaaactcaagccagtggattatgagtaccgagaagaagtccactgggccacgcaccagctccgcctgggcagaggctccttcggagaggtgcacaggatggaggacaagcagactggcttccagtgcgctgtcaaaaaggtgcggctggaagtatttcgggcagaggagctgatggcatgtgcaggattgacctcacccagaattgtccctttgtatggagctgtgagagaagggccttgggtcaacatcttcatggagctgctggaaggtggctccctgggccagctggtcaaggagcagggctgtctcccagaggaccgggccctgtactacctgggccaggccctggagggtctggaatacctccactcacgaaggattctgcatggggacgtcaaagctgacaacgtgctcctgtccagcgatgggagccacgcagccctctgtgactttggccatgctgtgtgtcttcaacctgatggcctgggaaagtccttgctcacaggggactacatccctggcacagagacccacatggctccggaggtggtgctgggcaggagctgcgacgccaaggtggatgtctggagcagctgctgtatgatgctgcacatgctcaacggctgccacccctggactcagttcttccgagggccgctctgcctcaagattgccagcgagcctccgcctgtgagggagatcccaccctcctgcgcccctctcacagcccaggccatccaagaggggctgaggaaagagcccatccaccgcgtgtctgcagcggagctgggagggaaggtgaaccgggcactacagcaagtgggaggtctgaagagcccttggaggggagaatataaagaaccaagacatccaccgccaaatcaagccaattaccaccagaccctccatgcccagccgagagagctttcgccaagggccccagggccccggccagctgaggagacaacaggcagagcccctaagctccagcctcctctcccaccagagcccccagagccaaacaagtctcctcccttgactttgagcaaggaggagtctgggatgtgggaacccttacctctgtcctccctggagccagcccctgccagaaaccccagctcaccagagcggaaagcaaccgtcccggagcaggaactgcagcagctggaaatagaattattcctcaacagcctgtcccagccattttctctggaggagcaggagcaaattctctcgtgcctcagcatcgacagcctctccctgtcggatgacagtgagaagaacccatcaaaggcctctcaaagctcgcgggacaccctgagctcaggcgtacactcctggagcagccaggccgaggctcgaagctccagctggaacatggtgctggcccgggggcggcccaccgacaccccaagctatttcaatggtgtgaaagtccaaatacagtctcttaatggtgaacacctgcacatccgggagttccaccgggtcaaagtgggagacatcgccactggcatcagcagccagatcccagctgcagccttcagcttggtcaccaaagacgggcagcctgttcgctacgacatggaggtgccagactcgggcatcgacctgcagtgcacactggcccctgatggcagcttcgcctggagctggagggtcaagcatggccagctggagaacaggccctaa

SEQ ID NO: 29 - DNA, 1083 bp, Homo sapiens
  source 1..1083: mol_type = genomic DNA
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctga

SEQ ID NO: 30 - DNA, 1656 bp, Homo sapiens
  source 1..1656: mol_type = genomic DNA
atggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 31 - DNA, 1443 bp, Homo sapiens
  source 1..1443: mol_type = genomic DNA
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 32 - DNA, 2514 bp, Homo sapiens
  source 1..2514: mol_type = genomic DNA
atggctagcaaacgaaaatctacaactccatgcatggttcggacatcacaagtagtagaacaagatgtgcccgaggaagtagacagggccaaagagaaaggaatcggcacaccacagcctgacgtggccaaggacagttgggcagcagaacttgaaaactcttccaaagaaaacgaagtgatagaggtgaaatctatgggggaaagccagtccaaaaaactccaaggtggttatgagtgcaaatactgcccctactccacgcaaaacctgaacgagttcacggagcatgtcgacatgcagcatcccaacgtgattctcaaccccctctacgtgtgtgcagaatgtaacttcacaaccaaaaagtacgactccctatccgaccacaactccaagttccatcccggggaggccaacttcaagctgaagttaattaaacgcaataatcaaactgtcttggaacagtccatcgaaaccaccaaccatgtcgtgtccatcaccaccagtggccctggaactggtgacagtgattctgggatctcggtgagtaaaacccccatcatgaagcctggaaaaccaaaagcggatgccaagaaggtgcccaagaagcccgaggagatcacccccgagaaccacgtggaagggaccgcccgcctggtgacagacacagctgagatcctctcgagactcggcggggtggagctcctccaagacacattaggacacgtcatgccttctgtacagctgccaccaaatatcaaccttgtgcccaaggtccctgtcccactaaatactaccaaatacaactctgccctggatacaaatgccacgatgatcaactctttcaacaagtttccttacccgacccaggctgagttgtcctggctgacagctgcctccaaacacccagaggagcacatcagaatctggtttgccacccagcgcttaaagcatggcatcagctggtccccagaagaggtggaggaggcccggaagaagatgttcaacggcaccatccagtcagtacccccgaccatcactgtgctgcccgcccagttggcccccacaaaggtgacgcagcccatcctccagacggctctaccgtgccagatcctcggccagactagcctggtgctgactcaggtgaccagcgggtcaacaaccgtctcttgctcccccatcacacttgccgtggcaggagtcaccaaccatggccagaagagacccttggtgactccccaagctgcccccgaacccaagcgtccacacatcgctcaggtgccagagcccccacccaaggtggccaaccccccgctcacaccagccagtgaccgcaagaagacaaaggagcagatagcacatctcaaggccagctttctccagagccagttccctgacgatgccgaggtttaccggctcatcgaggtgactggccttgccaggagcgagatcaagaagtggttcagtgaccaccgatatcggtgtcaaaggggcatcgtccacatcaccagcgaatcccttgccaaagaccagttggccatcgcggcctcccgacacggtcgcacgtatcatgcgtacccagactttgccccccagaagttcaaagagaaaacacagggtcaggttaaaatcttggaagacagctttttgaaaagttcttttcctacccaagcagaactggatcggctaagggtggagaccaagctgagcaggagagagatcgactcctggttctcggagaggcggaagcttcgagacagcatggaacaagctgtcttggattccatggggtctggcaaaaaaggccaagatgtgggagcccccaatggtgctctgtctcgactcgaccagctctccggtgcccagttaacaagttctctgcccagcccttcgccagcaattgcaaaaagtcaagaacaggttcatctcctgaggagcacgtttgcaagaacccagtggcctactccccaggagtacgaccagttagcggccaagactggcctggtccgaactgagattgtgcgttggttcaaggagaacagatgcttgctgaaaacgggaaccgtgaagtggatggagcagtaccagcaccagcccatggcagatgatcacggctacgatgccgtagcaaggaaagcaacaaaacccatggccgagagcccaaagaacgggggtgatgtggttccacaatattacaaggaccccaaaaagctctgcgaagaggacttggagaagttggtgaccagggtaaaagtaggcagcgagccagcaaaagactgtttgccagcaaagccctcagaggccacctcagaccggtcagagggcagcagccgggacggccagggtagcgacgagaacgaggagtcgagcgttgtggattacgtggaggtgacggtcggggaggaggatgcgatctcagatagatcagatagctggagtcaggctgcggcagaaggtgtgtcggaactggctgaatcagactccgactgcgtccctgcagaggctggccaggcctag

SEQ ID NO: 33 - DNA, 567 bp, Homo sapiens
  source 1..567: mol_type = genomic DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 34 - DNA, 570 bp, Homo sapiens
  source 1..570: mol_type = genomic DNA
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 35 - DNA, 2016 bp, Homo sapiens
  source 1..2016: mol_type = genomic DNA
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 36 - DNA, 1569 bp, Homo sapiens
  source 1..1569: mol_type = genomic DNA
atgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatag

SEQ ID NO: 37 - DNA, 999 bp, Homo sapiens
  source 1..999: mol_type = genomic DNA
atggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtga

(19) United States
(12) Patent Application Publication
(10) Pub. No.: US 2024/0391884 A1
(43) Pub. Date: Nov. 28, 2024

(54) COMPOUNDS AND COMPOSITIONS FOR TREATING CONDITIONS ASSOCIATED WITH NLRP ACTIVITY

(71) Applicant: Novartis AG, Basel (CH)

(72) Inventors: Luigi FRANCHI, Ann Arbor, MI (US); Shomir GHOSH, Brookline, MA (US); Gary GLICK, Ann Arbor, MI (US); Jason KATZ, Newton, MA (US); Anthony William OPIPARI, Jr., Dexter, MI (US); William ROUSH, Boston, MA (US); Hans Martin SEIDEL, Concord, MA (US); Dong-Ming SHEN, Edison, NJ (US); Shankar VENKATRAMAN, Lansdale, PA (US); David Guenther WINKLER, Arlington, MA (US)

(21) Appl. No.: 18/779,915

(22) Filed: Jul. 22, 2024

Related U.S. Application Data
(62) Division of application No. 17/662,863, filed on May 11, 2022, now abandoned.
Application No. 17/662,863 is a division of application No. 16/632,850, filed on Jan. 21, 2020, now Pat. No. 11,370,763.
(60) Provisional application No. 62/573,935, filed on Oct. 18, 2017.
(60) Provisional application No. 62/536,352, filed on Jul. 24, 2017.

(51) Int. Cl. C07D 265/30; A61K 45/06; A61P 1/00; A61P 11/00; A61P 19/08; A61P 35/00; C07C 381/10; C07D 209/26; C07D 211/78; C07D 215/06; C07D 231/12; C07D 239/26; C07D 239/36; C07D 261/08; C07D 263/46; C07D 277/36; C07D 317/50; C07D 333/34; C07D 401/12; C07D 409/12; C07D 417/12

(52) U.S. Cl. CPC C07D 265/30; A61P 1/00; A61P 11/00; A61P 19/08; A61P 35/00; C07C 381/10; C07D 209/26; C07D 211/78; C07D 215/06; C07D 231/12; C07D 239/26; C07D 239/36; C07D 261/08; C07D 263/46; C07D 277/36; C07D 317/50; C07D 333/34; C07D 401/12; C07D 409/12; C07D 417/12; A61K 45/06

(57) ABSTRACT

In one aspect, compounds of Formula AA, or a pharmaceutically acceptable salt thereof, are featured:

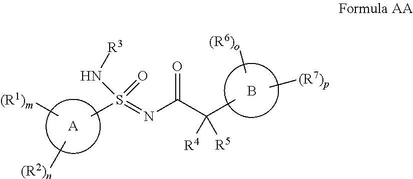

or a pharmaceutically acceptable salt thereof, wherein the variables shown in Formula A can be as defined anywhere herein.

### PRIORITY CLAIM

This application claims the benefit of U.S. Provisional Application No. 62/536,352, filed on Jul. 24, 2017; and U.S. Provisional Application No. 62/573,935, filed on Oct. 18, 2017; which are both incorporated herein by reference in their entirety.

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) is implicated.

In some embodiments, provided herein is a compound of Formula AA

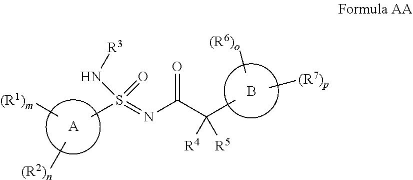

or a pharmaceutically acceptable salt thereof, wherein the variables in Formula AA can be as defined anywhere herein.

This disclosure also features compositions as well as other methods of using and making the same.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof;) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocyclic rings include five-membered, six-membered, and seven-membered heterocyclic rings.

The term “cycloalkyl” as used herein includes a nonaromatic cyclic, bicylic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to a nonaromatic 5-8 membered monocyclic, 8-12 membered bicyclic, or 11-14 membered tricyclic ring fused or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl, phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

A used herein, the terms “the ring A” or “A” are used interchangeably to denote

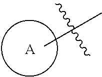

in formula AA, wherein the bond that is shown as being broken by the wavy line [symbol] connects A to the S(O)(NHR³)═N moiety of Formula AA.

As used herein, the terms “the ring B” or “B” are used interchangeably to denote

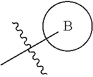

in formula AA wherein the bond that is shown as being broken by the wavy line [symbol] connects B to the C(R⁴R⁵) group of Formula AA.

As used herein, the term “the substituted ring A” is used to denote

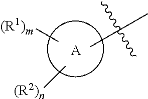

in formula AA, wherein the bond that is shown as being broken by the wavy line [symbol] connects A to the S(O)(NHR³)═N moiety of Formula AA.

As used herein, the term “the substituted ring B” is used to denote

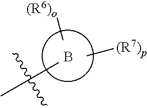

in formula AA, wherein the bond that is shown as being broken by the wavy line [symbol] connects B to the C(R⁴R⁵) group of Formula AA.

As used herein, the recitation “S(O₂)”, alone or as part of a larger recitation, refers to the group

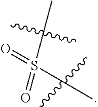 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The scope of the compounds disclosed herein includes tautomeric form of the compounds. Thus, by way of example, a compound that is represented as containing the moiety

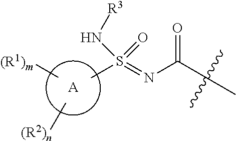

is also intended to include the tautomeric form containing the moiety

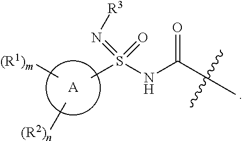

In addition, by way of example, a compound that is represented as containing the moiety

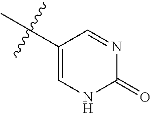 [SMILES: *c1cnc(=O)[nH]c1]

is also intended to include the tautomeric form containing the moiety

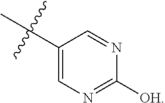 [SMILES: *c1cnc(O)nc1]

Non-limiting exemplified compounds of the formulae described herein include a stereogenic sulfur atom and optionally one or more stereogenic carbon atoms. This disclosure provides examples of stereoisomer mixtures (e.g., racemic mixture of enantiomers; mixture of diastereomers). This disclosure also describes and exemplifies methods for separating individual components of said stereoisomer mixtures (e.g., resolving the enantiomers of a racemic mixture). In cases of compounds containing only a stereogenic sulfur atom, resolved enantiomers are graphically depicted using one of the two following formats: formulas A/B (hashed and solid wedge three-dimensional representation); and formula C (“flat structures with *-labelled stereogenic sulfur).

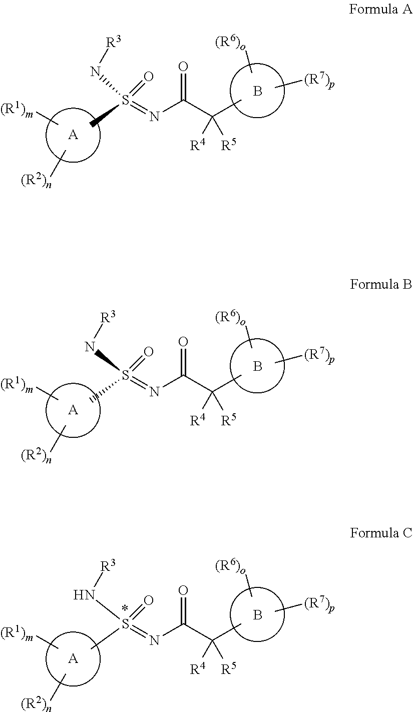

In reaction schemes showing resolution of a racemic mixture, Formulas A/B and C are intended only to convey that the constituent enantiomers were resolved in enantiopure pure form (about 98% ee or greater). The schemes that show resolution products using the formula A/B format are not intended to disclose or imply any correlation between absolute configuration and order of elution. Some of the compounds shown in the tables below are graphically represented using the formula A/B format. However, with the exception of compounds 132a and 132b, the depicted stereochemistry shown for each of the tabulated compounds drawn in the formula A/B format is a tentative assignment and based, by analogy, on the absolute stereochemistry assigned to compounds 132b (see, e.g., FIG. 1).

The details of one or more embodiments of the invention are set forth in the accompanying drawings and the description below. Other features and advantages of the invention will be apparent from the description and drawings, and from the claims.

### DESCRIPTION OF DRAWINGS

FIG. 1 depicts ball-and-stick representations of two crystallographically independent molecules of compound 132b in the asymmetrical unit.

### DETAILED DESCRIPTION

In some embodiments, provided herein is a compound of Formula AA

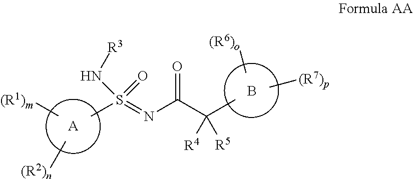

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl; wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 4- to 8-membered heterocyclic ring containing 1 or 2 heteroatoms independently selected from O, N, NR²⁰, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, NH—(C=NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆alkoxy, C₁-C₆alkyl, and,

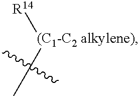

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments, provided herein is a compound of Formula AA

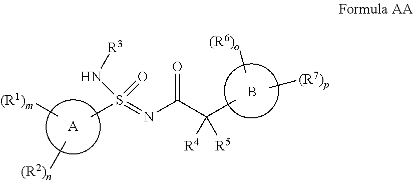

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA; R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR¹R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, NH—(C=NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆alkoxy, C₁-C₆alkyl, and

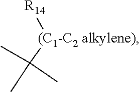

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments, provided herein is a compound of Formula AA

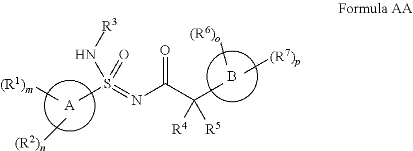

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl:
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
each of R⁴ and R is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆alkoxy, C₁-C₆alkyl, and

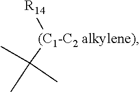

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments, provided herein is a compound of Formula AA

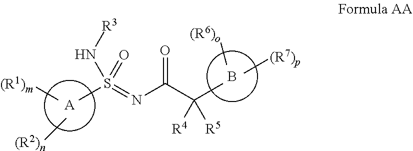

wherein
m=0, 1 or 2
n=0, 1 or 2
o=1 or 2
p=0, 1, 2 or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl; wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein
the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl; and R³ is selected from hydrogen, cyano, hydroxy, C₁-C₆alkoxy, C₁-C₆alkyl, CO₂C₁-C₆alkyl, and

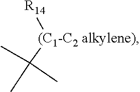

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶
or a pharmaceutically acceptable salt thereof.

In some embodiments, provided herein is a compound of Formula AA

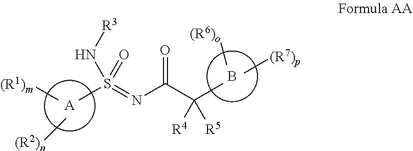

wherein
m=0, 1, or 2;
n=0, 1, or 2;
o=1 or 2;
p=0, 1, 2, or 3;
wherein
A is a 5-10-membered heteroaryl or a C₆-C₁₀ aryl;
B is a 5-10-membered heteroaryl or a C₆-C₁₀ aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl, 3- to 10-membered heterocycloalkyl, and C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl, 3- to 7-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxy, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆alkoxy, C₁-C₆ alkyl, and

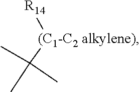

wherein the C₁-C₂ alkylene group is optionally substituted by oxo;
and
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶;
or a pharmaceutically acceptable salt thereof.

In some embodiments, provided herein is a compound of Formula AA

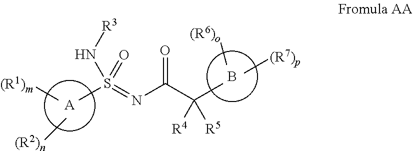

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl; and
R³ is selected from hydrogen, cyano, hydroxy, C₁-C₆alkoxy, C₁-C₆ alkyl, CO₂C₁-C₆ alkyl, and

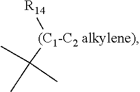

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶;
or a pharmaceutically acceptable salt thereof.

Provided herein is a compound of Formula AA

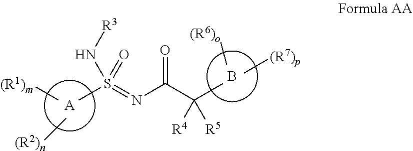

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA; R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₁-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl; R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl; and
R³ is selected from hydrogen, cyano, hydroxy, C₁-C₆alkoxy, C₁-C₆alkyl, CO₂C₁-C₆ alkyl, and

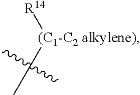

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶;
or a pharmaceutically acceptable salt thereof.

In some embodiments, provided herein is a compound of Formula AA

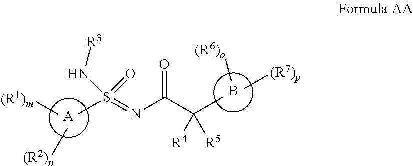

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
each of R⁸ and R⁹ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆alkoxy, C₁-C₆alkyl, and

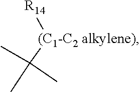

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments the variables shown in the formulae herein are as follows:

### The Variables m and n

In some embodiments m=0, 1, or 2.

In some embodiments m=0 or 1.

In some embodiments m=1 or 2.

In some embodiments m=0 or 2.

In some embodiments m=0.

In some embodiments m=1.

In some embodiments m=2.

In some embodiments n=0, 1, or 2.

In some embodiments n=0 or 1.

In some embodiments n=1 or 2.

In some embodiments n=0 or 2.

In some embodiments n=0.

In some embodiments n=1.

In some embodiments n=2.

In some embodiments, m=0 and n=0.

In some embodiments, m=1 and n=0.

In some embodiments, m=1 and n=1.

### The Ring A and Substitutions on the Ring A

In some embodiments, A is a 5-10-membered (e.g., 5-6-membered) monocyclic or bicyclic heteroaryl or a C₆-C₁₀ monocyclic or bicyclic aryl, such as phenyl.

In some embodiments, A is a 5-10-membered (e.g., 5-6-membered) monocyclic or bicyclic heteroaryl.

In some embodiments, A is a 5-membered heteroaryl containing a sulfur and optionally one or more nitrogens.

In some embodiments, A is a C₆-C₁₀ (e.g., C₆) monocyclic or bicyclic aryl.

In some embodiments, A is phenyl optionally substituted with 1 or 2 R¹ and optionally substituted with 1 or 2 R².

In some embodiments, A is furanyl optionally substituted with 1 or 2 R¹ and optionally substituted with 1 or 2 R².

In some embodiments, A is thiophenyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is thiazolyl optionally substituted with 1 or 2 R¹ and optionally substituted with 1 or 2 R².

In some embodiments, A is pyrazolyl optionally substituted with 1 or 2 R¹ and optionally substituted with 1 or 2 R².

In some embodiments, A is pyridyl optionally substituted with 1 or 2 R¹ and optionally substituted with 1 or 2 R².

In some embodiments, A is phenyl substituted with 1 R¹ and optionally substituted with 1 R².

In some embodiments, A is furanyl substituted with 1 R¹ and optionally substituted with 1 R².

In some embodiments, A is thiophenyl substituted with 1 R¹ and optionally substituted with 1 R².

In some embodiments, A is oxazolyl substituted with 1 R¹ and optionally substituted with 1 R².

In some embodiments, A is thiazolyl substituted with 1 R¹ and optionally substituted with 1 R².

In some embodiments, A is pyrazolyl substituted with 1 R¹ and optionally substituted with 1 R².

In some embodiments, A is pyridyl substituted with 1 R¹ and optionally substituted with 1 R².

In some embodiments, A is phenyl substituted with 1 R¹ and substituted with 1 R².

In some embodiments, A is furanyl substituted with 1 R¹ and substituted with 1 R².

In some embodiments, A is thiophenyl substituted with 1 R¹ and substituted with 1 R².

In some embodiments, A is oxazolyl substituted with 1 R¹ and substituted with 1 R².

In some embodiments, A is thiazolyl substituted with 1 R¹ and substituted with 1 R².

In some embodiments, A is pyrazolyl substituted with 1 R¹ and substituted with 1 R².

In some embodiments, A is pyridyl substituted with 1 R¹ and substituted with 1 R².

In some embodiments, A is phenyl, m is 0, 1, or 2; and n is 0, 1, or 2.

In some embodiments, A is furanyl, m is 0, 1, or 2, and n is 0, 1, or 2.

In some embodiments, A is thiophenyl, m is 0, 1, or 2, and n is 0, 1, or 2.

In some embodiments, A is oxazolyl, m is 0, 1, or 2, and n is 0, 1, or 2.

In some embodiments, A is thiazolyl, m is 0, 1, or 2, and n is 0, 1, or 2.

In some embodiments, A is pyrazolyl, m is 0, 1, or 2, and n is 0, 1, or 2.

In some embodiments, A is pyridyl m is 0, 1, or 2, and n is 0, 1, or 2.

In some embodiments, A is phenyl, m is 0 or 1, and n is 0 or 1.

In some embodiments, A is furanyl, m is 0 or 1, and n is 0 or 1.

In some embodiments, A is thiophenyl, m is 1 and n is 0 or 1.

In some embodiments, A is oxazolyl, m is 1 and n is 0 or 1.

In some embodiments, A is thiazolyl, m is 1 and n is 0 or 1.

In some embodiments, A is pyrazolyl, m is 1 and n is 0 or 1.

In some embodiments, A is pyridyl, m is 1 and n is 0 or 1.

In some embodiments, A is phenyl, m is 1 and n is 1.

In some embodiments, A is furanyl, m is 1 and n is 1.

In some embodiments, A is thiophenyl, m is 1 and n is 1.

In some embodiments, A is oxazolyl, m is 1 and n is 1.

In some embodiments, A is thiazolyl, m is 1 and n is 1.

In some embodiments, A is pyrazolyl, m is 1 and n is 1.

In some embodiments, A is pyridyl, m is 1 and n is 1.

In some embodiments, A is phenyl, m is 0 or 1, and n is 0, 1, or 2.

In some embodiments, A is furanyl, m is 0 or 1, and n is 0, 1, or 2.

In some embodiments, A is thiophenyl, m is 0 or 1, and n is 0, 1, or 2.

In some embodiments, A is oxazolyl, m is 0 or 1, and n is 0, 1, or 2.

In some embodiments, A is thiazolyl, m is 0 or 1, and n is 0, 1, or 2.

In some embodiments, A is pyrazolyl, m is 0 or 1, and n is 0, 1, or 2.

In some embodiments, A is pyridyl, m is 0 or 1, and n is 0, 1, or 2.

In some embodiments, A is phenyl, m is 0, and n is 0 or 1.

In some embodiments, A is furanyl, m is 0, and n is 0 or 1.

In some embodiments, A is thiophenyl, m is 0, and n is 0 or 1.

In some embodiments, A is oxazolyl, m is 0, and n is 0 or 1.

In some embodiments, A is thiazolyl, m is 0, and n is 0 or 1.

In some embodiments, A is pyrazolyl, m is 0, and n is 0 or 1.

In some embodiments, A is pyridyl, m is 0, and n is 0 or 1.

In some embodiments, A is one of the rings disclosed hereinbelow optionally substituted as disclosed hereinbelow, wherein in each case the bond that is shown as being broken by the wavy line [symbol] connects A to the S(O)(NHR³)═N moiety of Formula AA.

In some embodiments, the optionally substituted ring A is

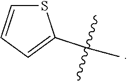 [SMILES: *c1cccs1]

In some embodiments, the optionally substituted ring A is

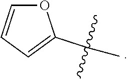 [SMILES: *c1ccco1]

In some embodiments, the optionally substituted ring A is

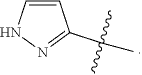 [SMILES: *c1cc[nH]n1]

In some embodiments, the optionally substituted ring A is

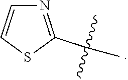 [SMILES: *c1nccs1]

In some embodiments, the optionally substituted ring A is

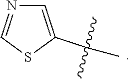 [SMILES: *c1cncs1]

In some embodiments, the optionally substituted ring A is

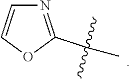 [SMILES: *c1ncco1]

In some embodiments, the optionally substituted ring A is

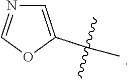 [SMILES: *c1cnco1]

In some embodiments, the optionally substituted ring A is

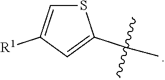

In some embodiments, the optionally substituted ring A is

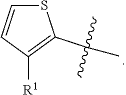

In some embodiments, the optionally substituted ring A is

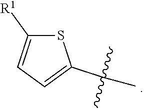

In some embodiments, the optionally substituted ring A is

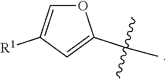

In some embodiments, the optionally substituted ring A is

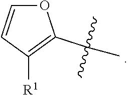

In some embodiments, the optionally substituted ring A is

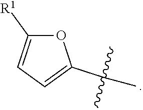

In some embodiments, the optionally substituted ring A is

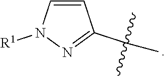

In some embodiments, the optionally substituted ring A is

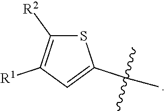

In some embodiments, the optionally substituted ring A is

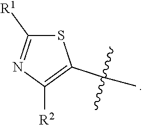

In some embodiments, the optionally substituted ring A is

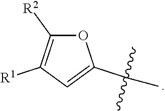

In some embodiments, the optionally substituted ring A is

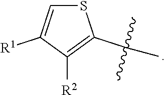

In some embodiments, the optionally substituted ring A is

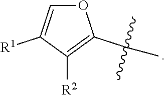

In some embodiments, the optionally substituted ring A is

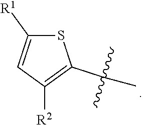

In some embodiments, the optionally substituted ring A is

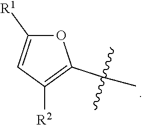

In some embodiments, the optionally substituted ring A is

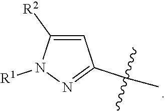

In some embodiments, the optionally substituted ring A is

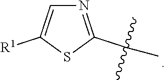

In some embodiments, the optionally substituted ring A is

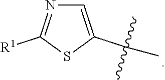

In some embodiments, the optionally substituted ring A is

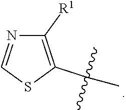

In some embodiments, the optionally substituted ring A is

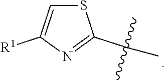

In some embodiments, the optionally substituted ring A is

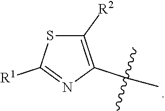

In some embodiments, the optionally substituted ring A is

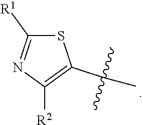

In some embodiments, the optionally substituted ring A is

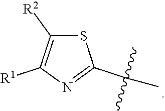

In some embodiments, the optionally substituted ring A is

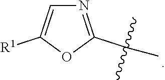

In some embodiments, the optionally substituted ring A is

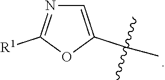

In some embodiments the optionally substituted ring A is

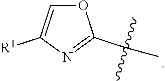

In some embodiments, the optionally substituted ring A is

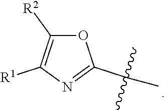

In some embodiments, the optionally substituted ring A is

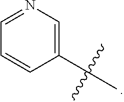 [SMILES: *c1cccnc1]

In some embodiments, the optionally substituted ring A is

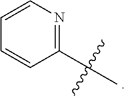 [SMILES: *c1ccccn1]

In some embodiments, the optionally substituted ring A is

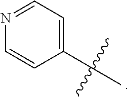 [SMILES: *c1ccncc1]

In some embodiments, the optionally substituted ring A is

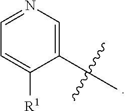

In some embodiments, the optionally substituted ring A is

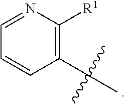

In some embodiments, the optionally substituted ring A is

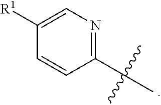

In some embodiments, the optionally substituted ring A is

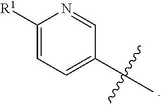

In some embodiments, the optionally substituted ring A is

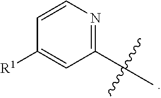

In some embodiments, the optionally substituted ring A is

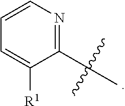

In some embodiments, the optionally substituted ring A is

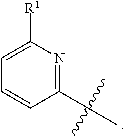

In some embodiments, the optionally substituted ring A is

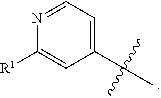

In some embodiments, the optionally substituted ring A is

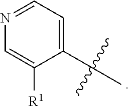

In some embodiments, the optionally substituted ring A is

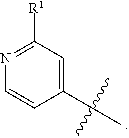

In some embodiments, the optionally substituted ring A is

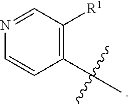

In some embodiments, the optionally substituted ring A is

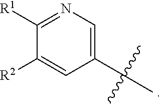

In some embodiments, the optionally substituted ring A is

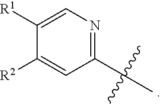

In some embodiments, the optionally substituted ring A is

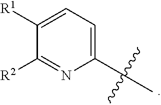

In some embodiments, the optionally substituted ring A is

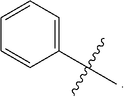 [SMILES: *c1ccccc1]

In some embodiments, the optionally substituted ring A is

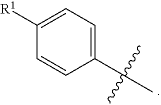

In some embodiments, the optionally substituted ring A is

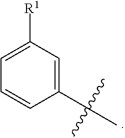

In some embodiments, the optionally substituted ring A is

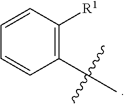

In some embodiments, the optionally substituted ring A is

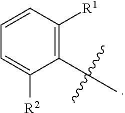

In some embodiments, the optionally substituted ring A is

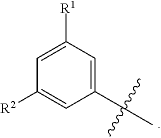

In some embodiments, the optionally substituted ring A is

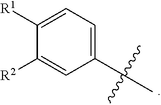

In some embodiments, the optionally substituted ring A is

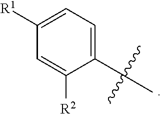

In some embodiments, the optionally substituted ring A is

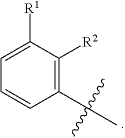

In some embodiments, the optionally substituted ring A is

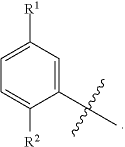

In some embodiments, the optionally substituted ring A is

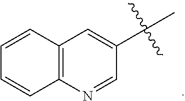 [SMILES: *c1cnc2ccccc2c1]

In some embodiments, the optionally substituted ring A is

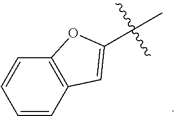 [SMILES: *c1cc2ccccc2o1]

In some embodiments, the optionally substituted ring A is

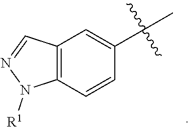

The Groups R¹ and R²

In some embodiments,
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, SC₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R¹ and R² are each independently selected from C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R¹ and R² are each independently selected from C₁-C₆ alkyl, halo, CN, NO₂, COC₁-C₆ alkyl, CO—C₆-C₁₀ aryl, CO-5- to 10-membered heteroaryl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is unsubstituted;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments,
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl are each unsubstituted;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R¹ and R² are each independently selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, each of R¹ and R² is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl: CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl: 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃: COCH₂CHM; 2-methoxy-2-propyl; (dimethylamino)methyl; 1-(dimethylamino)ethyl; fluoro; chloro; phenyl; pyridyl; pyrazolyl; S(O₂)CH; and S(O₂)NR¹¹R¹².

In some embodiments, R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH; and S(O₂)NR¹¹R¹².

In some embodiments, m=1; n=0; and
R¹ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NI-12, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=0; and,
R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=0; and,
R¹ is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=1; and
R¹ and R² are each independently selected from C₁-C₆ alkyl, C₁-C₆ haloalkyl, C₁-C₆ alkoxy, C₁-C₆ haloalkoxy, halo, CN, NO₂, CO—C₆-C₁₀ aryl, CO-5- to 10-membered heteroaryl, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=1; and,
R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=1; and
R¹ and R² are on adjacent atoms, and taken together with the atoms connecting them, form a C₄-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and
R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and
R¹ and R² are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and
R¹ and R² are on adjacent atoms, and taken together with the atoms connecting them, form a C₅ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and
R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

In some embodiments, m=1; n=1; and
R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.
   Particular Embodiments Wherein m=1 and n=0:

In some embodiments, R¹ is C₁-C₆ alkyl optionally substituted with one or more hydroxy.

In some embodiments, R¹ is 1-hydroxy-2-methylpropan-2-yl.

In some embodiments, R¹ is C₁-C₆ alkyl.

In some embodiments, R¹ is methyl.

In some embodiments, R¹ is isopropyl.

In some embodiments, R¹ is C₁-C₆ alkyl substituted with hydroxy at the carbon directly connected to ring A.

In some embodiments, R¹ is 2-hydroxy-2-propyl.

In some embodiments, R¹ is hydroxymethyl.

In some embodiments, R¹ is 1-hydroxyethyl.

In some embodiments, R¹ is 2-hydroxyethyl.

In some embodiments, R¹ is 1-hydroxy-2-propyl.

In some embodiments, R¹ is C₃-C₇ cycloalkyl optionally substituted with one or more hydroxy.

In some embodiments, R¹ is C₃-C₇ cycloalkyl.

In some embodiments, R¹ is C₃-C₇ cycloalkyl substituted with hydroxy at the carbon directly connected to ring A.

In some embodiments, R¹ is 1-hydroxy-1-cyclopropyl.

In some embodiments, R¹ is 1-hydroxy-1-cyclobutyl.

In some embodiments, R¹ is 1-hydroxy-1-cyclopentyl.

In some embodiments, R¹ is 1-hydroxy-1-cyclohexyl.

In some embodiments, R¹ is 3- to 7-membered heterocycloalkyl optionally substituted with one or more hydroxy.

In some embodiments, R¹ is 3- to 7-membered heterocycloalkyl.

In some embodiments, R¹ is morpholinyl (e.g., 4-morpholinyl).

In some embodiments, R¹ is 1,3-dioxolan-2-yl.

In some embodiments, R¹ is 3- to 7-membered heterocycloalkyl substituted with hydroxy at the carbon directly connected to ring A.

In some embodiments, R¹ is C₁-C₆ alkyl optionally substituted with one or more oxo.

In some embodiments, R¹ is COCH₃.

In some embodiments, R¹ is COCH₂CH₃.

In some embodiments, R¹ is C₃-C₇ cycloalkyl optionally substituted with one or more oxo.

In some embodiments, R¹ is 3- to 7-membered heterocycloalkyl optionally substituted with one or more oxo.

In some embodiments, R¹ is C₁-C₆ alkyl optionally substituted with one or more C₁-C₆ alkoxy.

In some embodiments, R¹ is 2-methoxy-2-propyl.

In some embodiments, R¹ is C₃-C₇ cycloalkyl optionally substituted with one or more C₁-C₆ alkoxy.

In some embodiments, R¹ is 3- to 7-membered heterocycloalkyl optionally substituted with one or more C₁-C₆ alkoxy.

In some embodiments, R¹ is C₁-C₆ alkyl optionally substituted with one or more NR⁸R⁹.

In some embodiments, R¹ is (dimethylamino)methyl.

In some embodiments, R¹ is 2-(dimethylamino)prop-2-yl.

In some embodiments, R¹ is C₃-C₇ cycloalkyl optionally substituted with one or more NR⁸R⁹.

In some embodiments, R¹ is 3- to 7-membered heterocycloalkyl optionally substituted with one or more NR⁸R⁹.

In some embodiments, R¹ is C₁-C₆ alkyl optionally substituted with one or more hydroxy and one or more oxo.

In some embodiments, R¹ is C(Me)₂C(O)OH.

In some embodiments, R¹ is C₁-C₆ haloalkyl optionally substituted with one or more hydroxy.

In some embodiments, R¹ is C₁-C₆ alkoxy.

In some embodiments, R¹ is C₁-C₆ haloalkoxy.

In some embodiments, R¹ is halo.

In some embodiments, R¹ is fluoro.

In some embodiments, R¹ is chloro.

In some embodiments, R¹ is CN.

In some embodiments, R¹ is NO₂.

In some embodiments, R¹ is COC₁-C₆ alkyl.

In some embodiments, R¹ is CO—C₆-C₁₀ aryl.

In some embodiments, R¹ is CO-5- to 10-membered heteroaryl.

In some embodiments, R¹ is CO₂C₁-C₆ alkyl.

In some embodiments, R¹ is CO₂C₃-C₈ cycloalkyl.

In some embodiments, R¹ is OCOC₁-C₆ alkyl.

In some embodiments, R¹ is OCOC₆-C₁₀ aryl.

In some embodiments, R¹ is OCO(5- to 10-membered heteroaryl).

In some embodiments, R¹ is OCO(3- to 7-membered heterocycloalkyl).

In some embodiments, R¹ is C₆-C₁₀ aryl.

In some embodiments, R¹ is phenyl.

In some embodiments, R¹ is 5- to 10-membered heteroaryl.

In some embodiments, R¹ is pyridyl (e.g., 4-pyridyl).

In some embodiments, R¹ is pyrazolyl (e.g., 1-pyrazolyl).

In some embodiments, R¹ is NH₂.

In some embodiments, R¹ is NHC₁-C₆ alkyl.

In some embodiments, R¹ is N(C₁-C₆ alkyl)₂.

In some embodiments, R¹ is CONR⁸R⁹.

In some embodiments, R¹ is SF₅.

In some embodiments, R¹ is SC₁-C₆ alkyl,

In some embodiments, R¹ is S(O₂)C₁-C₆ alkyl.

In some embodiments, R¹ is S(O₂)CH₃.

In some embodiments, R¹ is S(O₂)NR¹¹R¹².

In some embodiments, R¹ is S(O₂)N(CH₃)₂.

In some embodiments, R¹ is S(O)C₁-C₆ alkyl.

In some embodiments, R¹ is S(O)CH.

In some embodiments, R¹ is attached to a carbon of an aryl ring A.

In some embodiments, R¹ is attached to a carbon of a heteroaryl ring A.

In some embodiments, R¹ is attached to a nitrogen of a heteroaryl ring A.

Particular Embodiments Wherein m=1 and n=1:

In some embodiments, R¹ is C₁-C₆ alkyl optionally substituted with one or more hydroxy, and R² is C₁-C₆ alkyl optionally substituted with one or more hydroxy.

In some embodiments, R¹ is 1-hydroxy-2-methylpropan-2-yl, and R² is methyl.

In some embodiments, R¹ is 2-hydroxy-2-propyl and R² is methyl.

In some embodiments, R¹ is 2-hydroxy-2-propyl and R² is isopropyl.

In some embodiments, R¹ is 2-hydroxy-2-propyl and R² is 2-hydroxy-2-propyl.

In some embodiments, R¹ is 2-hydroxy-2-propyl and R² is 1-hydroxyethyl.

In some embodiments, R¹ is hydroxymethyl and R² is methyl.

In some embodiments, R¹ is 1-hydroxyethyl and R² is methyl.

In some embodiments, R¹ is 2-hydroxyethyl and R² is methyl.

In some embodiments, R¹ is 1-hydroxy-2-propyl and R² is methyl.

In some embodiments, R¹ is C₁-C₆ alkyl optionally substituted with one or more hydroxy, and R² is C₆-C₁₀ aryl.

In some embodiments, R¹ is 2-hydroxy-2-propyl and R² is phenyl.

In some embodiments, R¹ is C₁-C₆ alkyl optionally substituted with one or more hydroxy, and R² is 5- to 10-membered heteroaryl.

In some embodiments, R¹ is 2-hydroxy-2-propyl and R² is pyridyl.

In some embodiments, R¹ is 2-hydroxy-2-propyl and R² is pyrazolyl.

In some embodiments, R¹ is C₁-C₆ alkyl optionally substituted with one or more hydroxy, and R² is SF₅.

In some embodiments, R¹ is C₁-C₆ alkyl optionally substituted with one or more hydroxy, and R² is SC₁-C₆ alkyl.

In some embodiments, R¹ is C₁-C₆ alkyl optionally substituted with one or more hydroxy, and R² is S(O₂)C₁-C₆ alkyl.

In some embodiments, R¹ is C₁-C₆ alkyl optionally substituted with one or more hydroxy, and R² is S(O₂)CH₃.

In some embodiments, R¹ is C₁-C₆ alkyl optionally substituted with one or more hydroxy, and R² is halo.

In some embodiments, R¹ is 2-hydroxy-2-propyl and R² is chloro.

In some embodiments, R¹ is 2-hydroxy-2-propyl and R² is fluoro.

In some embodiments, R¹ is C₃-C₇ cycloalkyl optionally substituted with one or more hydroxy, and R² is C₁-C₆ alkyl.

In some embodiments, R¹ is 1-hydroxy-1-cyclopropyl, and R² is methyl.

In some embodiments, R¹ is 1-hydroxy-1-cyclobutyl, and R² is methyl.

In some embodiments, R¹ is 1-hydroxy-1-cyclopentyl, and R² is methyl.

In some embodiments, R¹ is 1-hydroxy-1-cyclohexyl, and R² is methyl.

In some embodiments, R¹ is 3- to 7-membered heterocycloalkyl optionally substituted with one or more hydroxy, and R² is C₁-C₆ alkyl.

In some embodiments, R¹ is morpholinyl, and R² is methyl.

In some embodiments, R¹ is 1,3-dioxolan-2-yl, and R² is methyl.

In some embodiments, R¹ is 3- to 7-membered heterocycloalkyl optionally substituted with one or more hydroxy, and R² is halo.

In some embodiments, R¹ is 1,3-dioxolan-2-yl, and R² is fluoro.

In some embodiments, R¹ is 1,3-dioxolan-2-yl, and R² is chloro.

In some embodiments, R¹ is C₁-C₆ alkyl optionally substituted with one or more oxo, and R² is methyl.

In some embodiments, R¹ is COCH₃, and R² is methyl.

In some embodiments, R¹ is C₁-C₆ alkyl optionally substituted with one or more C₁-C₆ alkoxy, and R² is C₁-C₆ alkyl.

In some embodiments, R¹ is 2-methoxy-2-propyl, and R² is methyl.

In some embodiments, R¹ is C₁-C₆ alkyl optionally substituted with one or more NR⁸R⁹, and R² is C₁-C₆ alkyl.

In some embodiments, R¹ is (dimethylamino)methyl, and R² is methyl.

In some embodiments, R¹ is C₁-C₆ alkyl optionally substituted with one or more NR⁸R⁹, and R² is halo.

In some embodiments, R¹ is (dimethylamino)methyl, and R² is fluoro.

In some embodiments, R² is 1-hydroxy-2-methylpropan-2-yl, and R¹ is methyl.

In some embodiments, R² is 2-hydroxy-2-propyl and R¹ is methyl.

In some embodiments, R² is 2-hydroxy-2-propyl and R¹ is isopropyl.

In some embodiments, R² is 2-hydroxy-2-propyl and R¹ is 1-hydroxyethyl.

In some embodiments, R² is hydroxymethyl and R¹ is methyl.

In some embodiments, R² is 1-hydroxyethyl and R¹ is methyl.

In some embodiments, R² is 2-hydroxyethyl and R¹ is methyl.

In some embodiments, R² is 1-hydroxy-2-propyl and R¹ is methyl.

In some embodiments, R² is C₁-C₆ alkyl optionally substituted with one or more hydroxy, and R¹ is C₆-C₁₀ aryl.

In some embodiments, R² is 2-hydroxy-2-propyl and R¹ is phenyl.

In some embodiments, R² is C₁-C₆ alkyl optionally substituted with one or more hydroxy, and R¹ is 5- to 10-membered heteroaryl.

In some embodiments, R² is 2-hydroxy-2-propyl and R¹ is pyridyl.

In some embodiments, R² is 2-hydroxy-2-propyl and R¹ is pyrazolyl.

In some embodiments, R² is C₁-C₆ alkyl optionally substituted with one or more hydroxy, and R¹ is SF₅.

In some embodiments, R² is C₁-C₆ alkyl optionally substituted with one or more hydroxy, and R¹ is SC₁-C₆ alkyl.

In some embodiments, R² is C₁-C₆ alkyl optionally substituted with one or more hydroxy, and R¹ is S(O₂)C₁-C₆ alkyl.

In some embodiments, R² is C₁-C₆ alkyl optionally substituted with one or more hydroxy, and R¹ is S(O₂)CH₃.

In some embodiments, R² is C₁-C₆ alkyl optionally substituted with one or more hydroxy, and R¹ is halo.

In some embodiments, R² is 2-hydroxy-2-propyl and R¹ is chloro.

In some embodiments, R² is 2-hydroxy-2-propyl and R¹ is fluoro.

In some embodiments, R² is C₃-C₇ cycloalkyl optionally substituted with one or more hydroxy, and R¹ is C₁-C₆ alkyl.

In some embodiments, R² is 1-hydroxy-1-cyclopropyl, and R¹ is methyl.

In some embodiments, R² is 1-hydroxy-1-cyclobutyl, and R¹ is methyl.

In some embodiments, R² is 1-hydroxy-1-cyclopentyl, and R¹ is methyl.

In some embodiments, R² is 1-hydroxy-1-cyclohexyl, and R¹ is methyl.

In some embodiments, R² is 3- to 7-membered heterocycloalkyl optionally substituted with one or more hydroxy, and R¹ is C₁-C₆ alkyl.

In some embodiments, R² is morpholinyl, and R¹ is methyl.

In some embodiments, R² is 1,3-dioxolan-2-yl, and R¹ is methyl.

In some embodiments, R² is 3- to 7-membered heterocycloalkyl optionally substituted with one or more hydroxy, and R¹ is halo.

In some embodiments, R² is 1,3-dioxolan-2-yl, and R¹ is fluoro.

In some embodiments, R² is 1,3-dioxolan-2-yl, and R¹ is chloro.

In some embodiments, R² is C₁-C₆ alkyl optionally substituted with one or more oxo, and R¹ is methyl.

In some embodiments, R² is COCH₃, and R¹ is methyl.

In some embodiments, R² is C₁-C₆ alkyl optionally substituted with one or more C₁-C₆ alkoxy, and R¹ is C₁-C₆ alkyl.

In some embodiments, R² is 2-methoxy-2-propyl, and R¹ is methyl.

In some embodiments, R² is C₁-C₆ alkyl optionally substituted with one or more NR⁸R⁹, and R¹ is C₁-C₆ alkyl.

In some embodiments, R² is (dimethylamino)methyl, and R¹ is methyl.

In some embodiments, R² is C₁-C₆ alkyl optionally substituted with one or more NR⁸R⁹, and R¹ is halo.

In some embodiments, R² is (dimethylamino)methyl, and R¹ is fluoro.

In some embodiments, R¹ and R² are each attached to a carbon of an aryl ring A.

In some embodiments, R¹ and R² are each attached to a carbon of a heteroaryl ring A.

In some embodiments, R¹ is attached to a carbon and R² is attached to a nitrogen of a heteroaryl ring A.

In some embodiments, R² is attached to a carbon and R¹ is attached to a nitrogen of a heteroaryl ring A.

In some embodiments, R¹ and R² are on adjacent atoms, and taken together with the atoms connecting them, form a C₈ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₈ aliphatic carbocyclic ring.

In some embodiments, R¹ and R² are on adjacent atoms, and taken together with the atoms connecting them, form a C₈ saturated carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₆ aliphatic carbocyclic ring.

In some embodiments, R¹ and R² are on adjacent atoms, and taken together with the atoms connecting them, form a C₆ saturated carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 5-membered aliphatic heterocyclic ring containing 1 or 2 heteroatoms independently selected from O, N, and S.

In some embodiments, R¹ and R² are on adjacent atoms, and taken together with the atoms connecting them, form a 5-membered heteroaromatic ring containing 1 or 2 heteroatoms independently selected from O, N, and S.

In some embodiments, R¹ and R² are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 6-membered aliphatic heterocyclic ring containing 1 or 2 heteroatoms independently selected from O, N, and S.

In some embodiments, R¹ and R² are on adjacent atoms, and taken together with the atoms connecting them, form a 6-membered heteroaromatic ring containing 1 or 2 heteroatoms independently selected from O, N, and S.

In some embodiments, R¹ and R² are different.

In some embodiments, R¹ and R² are different, and R² comprises a carbonyl group.

In some embodiments, R¹ and R² are different, and R² comprises 1 or 2 (e.g., 1) nitrogen atoms.

In some embodiments, R¹ and R² are different, and R² comprises 1 or 2 (e.g., 1) oxygen atoms.

In some embodiments, R¹ and R² are different, and R² comprises a sulfur atom.

In some embodiments, R² and R¹ are different, and R² comprises a carbonyl group.

In some embodiments, R² and R¹ are different, and R² comprises 1 or 2 (e.g., 1) nitrogen atoms.

In some embodiments, R² and R¹ are different, and R² comprises 1 or 2 (e.g., 1) oxygen atoms.

In some embodiments, R² and R¹ are different, and R² comprises a sulfur atom.

In some embodiments, R¹ and R² are the same.

In some embodiments, R¹ is para or meta to R².

In some embodiments, R¹ is para or ortho to R².

In some embodiments, R¹ is ortho or meta to R².

In some embodiments, R¹ is para to R².

In some embodiments, R¹ is meta to R².

In some embodiments, R¹ is ortho to R².

### The Variables o and p

In some embodiments o=1 or 2.

In some embodiments o=1.

In some embodiments o=2.

In some embodiments p=0, 1, 2, or 3.

In some embodiments p=0.

In some embodiments p=1.

In some embodiments p=2.

In some embodiments, o=1 and p=0.

In some embodiments, o=2 and p=0.

In some embodiments, o=1 and p=1.

In some embodiments, o=1 and p=2.

In some embodiments, o=2 and p=1.

In some embodiments, o=2 and p=2.

In some embodiments, o=2 and p=3.

### The Ring B and Substitutions on the Ring B

In some embodiments, B is a 5-10-membered monocyclic or bicyclic heteroaryl or a C₆-C₁₀ monocyclic or bicyclic aryl, such as phenyl.

In some embodiments, B is a 5-6-membered monocyclic heteroaryl or a C₆ monocyclic aryl.

In some embodiments, B is a 5-10-membered monocyclic or bicyclic heteroaryl.

In some embodiments, B is a C₆-C₁₀ monocyclic or bicyclic aryl.

In some embodiments, B is a 5-membered monocyclic or bicyclic heteroaryl.

In some embodiments, B is a 7-10 membered monocyclic or bicyclic heteroaryl.

In some embodiments, B is a 6-membered bicyclic heteroaryl.

In some embodiments, B is a 6-membered monocyclic heteroaryl containing 2 or more N atoms.

In some embodiments, B is phenyl, o is 1 or 2, and p is 0, 1, 2, or 3.

In some embodiments, B is pyridyl, o is 1 or 2, and p is 0, 1, 2, or 3.

In some embodiments, B is 3-pyridyl, o is 1 or 2, and p is 0, 1, 2, or 3.

In some embodiments, B is phenyl, o is 1, or 2, and p is 0, 1, 2, or 3.

In some embodiments, B is pyridyl (e.g., 3-pyridyl), o is 1 or 2, and p is 0, 1, 2, or 3.

In some embodiments, B is phenyl, o is 1, and p is 1, 2, or 3.

In some embodiments, B is phenyl, o is 2, and p is 1, 2, or 3.

In some embodiments, B is pyridyl (e.g., 3-pyridyl), o is 1, and p is 0, 1, 2, or 3.

In some embodiments, B is pyridyl (e.g., 3-pyridyl), o is 2, and p is 0, 1, 2, or 3.

In some embodiments, B is phenyl, o is 1 or 2, and p is 0, 1, 2, or 3.

In some embodiments, B is pyridyl (e.g., 3-pyridyl), o is 1 or 2, and p is 0, 1, 2, or 3.

In some embodiments, B is phenyl, o is 1, and p is 0, 1, 2, or 3.

In some embodiments, B is phenyl, o is 2, and p is 0, 1, 2, or 3.

In some embodiments, B is pyridyl (e.g., 3-pyridyl), o is 1, and p is 0, 1, 2, or 3.

In some embodiments, B is pyridyl (e.g., 3-pyridyl), o is 2, and p is 0, 1, or 2.

In some embodiments, B is pyrimidinyl (e.g., pyrimidin-5-yl), o is 1, and p is 0, 1, or 2.

In some embodiments, B is pyrimidinyl (e.g., pyrimidin-5-yl), o is 2, and p is 0 or 1.

In some embodiments, B is one of the rings disclosed hereinbelow, substituted as disclosed hereinbelow, wherein in each case the bond that is shown as being broken by the wavy line [symbol] connects B to the NH(CO) group of Formula AA.

In some embodiments, the substituted ring B

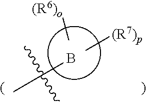

is

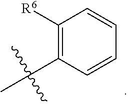

In some embodiments, the substituted ring B is

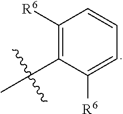

In some embodiments, the substituted ring B is

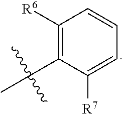

In some embodiments, the substituted ring B is

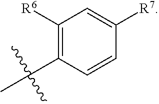

In some embodiments, the substituted ring B is

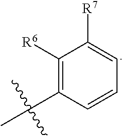

In some embodiments, the substituted ring B is

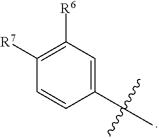

In some embodiments, the substituted ring B is

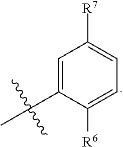

In some embodiments, the substituted ring B is

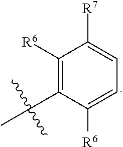

In some embodiments, the substituted ring B is

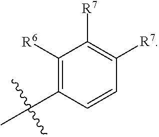

In some embodiments, the substituted ring B is

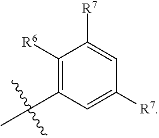

In some embodiments, the substituted ring B is

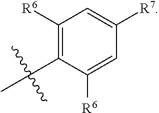

In some embodiments, the substituted ring B is

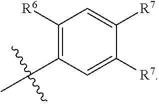

In some embodiments, the substituted ring B is

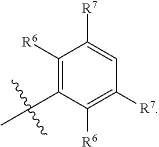

In some embodiments, the substituted ring B is

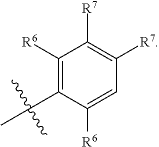

In some embodiments, the substituted ring B is

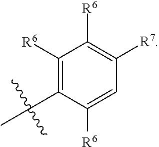

In some embodiments, the substituted ring B is

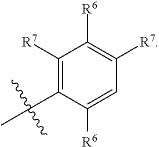

In some embodiments, the substituted ring B is

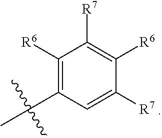

In some embodiments, the substituted ring B is

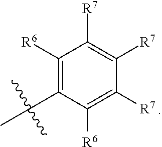

In some embodiments, the substituted ring B is

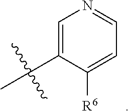

In some embodiments, the substituted ring B is

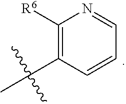

In some embodiments, the substituted ring B is

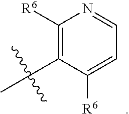

In some embodiments, the substituted ring B is

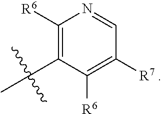

In some embodiments, the substituted ring B is

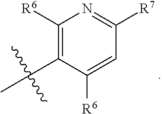

In some embodiments, the substituted ring B is

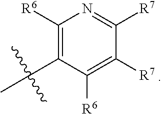

In some embodiments, the substituted ring B is

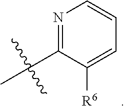

In some embodiments, the substituted ring B is

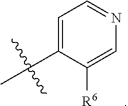

In some embodiments, the substituted ring B is

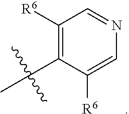

In some embodiments, the substituted ring B is

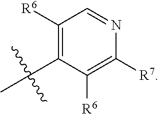

In some embodiments, the substituted ring B is

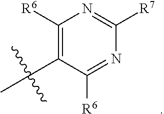

The Groups R¹ and R⁷

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from
hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, 5° C. OC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,
C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or
   wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₆ aliphatic carbocyclic ring or at least one 5- to 6-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₁-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, 5° C. OC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NI-12, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, =NRto, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₁-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₁₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl are each unsubstituted;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S,
wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,
R⁶ is independently selected from C₁-C₆alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy:
or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and Ron adjacent atoms, taken together with the atoms connecting them, independently form at least one 5- to 8-membered heterocyclic ring containing 1 or 2 heteroatoms independently selected from O, N, and S, wherein the heterocyclic ring is optionally independently substituted with one or more hydroxy or oxo.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,
at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,
at least one pair of R⁶ and Ron adjacent atoms, taken together with the atoms connecting them, independently form at least one C₄ aliphatic carbocyclic ring, wherein the carbocyclic ring is optionally independently substituted with one or more hydroxy or oxo.

In some embodiments,
at least one pair of R⁶ and R⁷ on adjacent atoms, taken together with the atoms connecting them, independently form at least one C₈ aliphatic carbocyclic ring, wherein the carbocyclic ring is optionally independently substituted with one or more hydroxy or oxo.

In some embodiments,
at least one pair of R⁶ and R⁷ on adjacent atoms, taken together with the atoms connecting them, independently form at least one C₆ aliphatic carbocyclic ring, wherein the carbocyclic ring is optionally independently substituted with one or more hydroxy or oxo.

In some embodiments,
at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more hydroxy or oxo.

In some embodiments,
at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more hydroxy or oxo.

In some embodiments,
at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more hydroxy or oxo.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more C₁-C₆ alkyl.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one 5- to 8-membered heterocyclic ring containing 1 or 2 heteroatoms independently selected from O, N, and S, wherein the heterocyclic ring is optionally independently substituted with one or more C₁-C₆ alkyl.

In some embodiments,
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from C₁-C₆ alkyl.
or at least one pair of R⁶ and R⁷ on adjacent atoms, taken together with the atoms connecting them, independently form at least one C₄-C₈ carbocyclic ring, wherein the carbocyclic ring is optionally independently substituted with one or more C₁-C₆ alkyl.

In some embodiments,
at least one pair of R⁶ and Ron adjacent atoms, taken together with the atoms connecting them, independently form at least one C₄-C₆ aliphatic carbocyclic ring, wherein the carbocyclic ring is optionally independently substituted with one or more C₁-C₆ alkyl.

In some embodiments,
at least one pair of R⁶ and R⁷ on adjacent atoms, taken together with the atoms connecting them, independently form at least one C₄ aliphatic carbocyclic ring, wherein the carbocyclic ring is optionally independently substituted with one or more C₁-C₆ alkyl.

In some embodiments,
at least one pair of R⁶ and Ron adjacent atoms, taken together with the atoms connecting them, independently form at least one C₈ aliphatic carbocyclic ring, wherein the carbocyclic ring is optionally independently substituted with one or more C₁-C₆ alkyl.

In some embodiments,
at least one pair of R⁶ and Ron adjacent atoms, taken together with the atoms connecting them, independently form at least one C₆ aliphatic carbocyclic ring, wherein the carbocyclic ring is optionally independently substituted with one or more C₁-C₆ alkyl.

In some embodiments,
at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more C₁-C₆ alkyl.

In some embodiments,
at least one pair of R⁶ and Ron adjacent atoms, taken together with the atoms connecting them, independently form at least one 5-membered heterocyclic ring containing 1 heteroatom independently selected from O, N, and S, wherein the heterocyclic ring is optionally independently substituted with one or more C₁-C₆ alkyl.

In some embodiments,
at least one pair of R⁶ and Ron adjacent atoms, taken together with the atoms connecting them, independently form at least one 6-membered heterocyclic ring containing 1 heteroatom independently selected from O, N, and S, wherein the heterocyclic ring is optionally independently substituted with one or more C₁-C₆ alkyl.

In some embodiments, o=1; p=0; and
R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5-to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and
R⁶ is selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments, o=1 or 2; p=1, 2, or 3; and
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R¹ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆alkyl is optionally substituted with one to two C₁-C₆ alkoxy;
or at least one pair of R⁶ and R⁷ on adjacent atoms, taken together with the atoms connecting them, independently form at least one C₄-C₇ (e.g., C₄-C₆) carbocyclic ring (e.g., aliphatic carbocyclic ring) or at least one 5-to-7-membered (e.g., 5-to-6-membered) heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments, o=1 or 2; p=1, 2, or 3; and
R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and
one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and
one R⁶ and one R⁷ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and
one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

In some embodiments, o=2; p=2 or 3; and
two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.
   Particular Embodiments Wherein o=1; p=0:

In some embodiments, R⁶ is C₁-C₆ alkyl.

In some embodiments, R⁶ is isopropyl.

In some embodiments, R⁶ is ethyl.

In some embodiments, R⁶ is methyl.

In some embodiments, R⁶ is C₁-C₆ alkyl substituted with one or more halo.

In some embodiments, R⁶ is C₁-C₆ alkyl (e.g., methyl) substituted with one or more (e.g., one) C₁-C₆ alkoxy (e.g., methoxy). In some embodiments, R⁶ is methoxymethyl.

In some embodiments, R⁶ is trifluoromethyl.

In some embodiments, R⁶ is trifluoromethoxy.

In some embodiments, R⁶ is C₃-C₇ cycloalkyl.

In some embodiments, R⁶ is cyclopropyl.

In some embodiments, R⁶ is halo.

In some embodiments, R⁶ is chloro.

In some embodiments, R⁶ is fluoro.

In some embodiments, R⁶ is cyano.

In some embodiments, R⁶ is attached to a carbon of an aryl ring B.

In some embodiments, R⁶ is attached to a carbon of a heteroaryl ring B.

In some embodiments, R⁶ is attached to a nitrogen of a heteroaryl ring B.

Particular embodiments wherein o=1 or 2; p=1, 2, or 3:

In some embodiments, at least one R⁶ is C₁-C₆ alkyl, and at least one R¹ is C₁-C₆ alkyl optionally substituted with one or more halo.

In some embodiments, at least one R⁶ is C₁-C₆ alkyl, and at least one R¹ is halo.

In some embodiments, at least one R⁶ is C₁-C₆ alkyl and at least one R¹ is C₁-C₆ alkyl.

In some embodiments, at least one R⁶ is isopropyl and at least one R¹ is methyl.

In some embodiments, at least one R⁶ is isopropyl and at least one R⁷ is isopropyl.

In some embodiments, o=1; p=1; R⁶ is isopropyl; and R⁷ is isopropyl.

In some embodiments, at least one R⁶ is C₁-C₆ alkyl, and at least one R¹ is C₁-C₆ alkyl substituted with one or more halo.

In some embodiments, at least one R⁶ is isopropyl and at least one R¹ is trifluoromethyl.

In some embodiments, at least one R⁶ is C₁-C₆ alkyl, and at least one R¹ is C₃-C₇ cycloalkyl.

In some embodiments, at least one R⁶ is isopropyl and at least one R⁷ is cyclopropyl.

In some embodiments, o=1; p=1; R⁶ is isopropyl; and R¹ is cyclopropyl.

In some embodiments, o=1; p=²; R⁶ is isopropyl; one R¹ is cyclopropyl; and the other R⁷ is fluoro.

In some embodiments, o=2; p=²; one R⁶ is isopropyl; one R¹ is cyclopropyl; the other R⁶ is cyano; and the other R¹ is fluoro.

In some embodiments, at least one R⁶ is C₁-C₆ alkyl, and at least one R¹ is halo.

In some embodiments, at least one R⁶ is isopropyl and at least one R¹ is halo.

In some embodiments, at least one R⁶ is isopropyl and at least one R⁷ is chloro.

In some embodiments, at least one R⁶ is isopropyl and at least one R⁷ is fluoro.

In some embodiments, o=1; p=1; R⁶ is isopropyl; and R¹ is chloro.

In some embodiments, o=2; p=1; at least one R⁶ is isopropyl; and R⁷ is chloro.

In some embodiments, o=1; p=1; R⁶ is isopropyl; and R¹ is fluoro.

In some embodiments, o=2; p=1; at least one R⁶ is isopropyl, and R⁷ is fluoro.

In some embodiments, o=2; p=1; each R⁶ is isopropyl, and R¹ is fluoro.

In some embodiments, o=1; p=1; R⁶ is isopropyl; and R¹ is fluoro.

In some embodiments, o=2; p=1; at least one R⁶ is isopropyl; and R¹ is fluoro.

In some embodiments, o=2; p=2; at least one R⁶ is isopropyl, one R⁷ is fluoro, and one R¹ is phenyl substituted with trifluoromethyl (e.g., substituted at the meta position with trifluoromethyl).

In some embodiments, o=2; p=²; at least one R⁶ is isopropyl; and R¹ is fluoro.

In some embodiments, o=2; p=2; at least one R⁶ is isopropyl; one R⁷ is fluoro; and the other R¹ is cyano.

In some embodiments, o=2; p=3; at least one R⁶ is isopropyl; two R¹ are fluoro; and one R⁷ is chloro.

In some embodiments, o=2; p=1; at least one R⁶ is ethyl; and R¹ is fluoro.

In some embodiments, o=2; p=1; at least one R⁶ is isopropyl; the other R⁶ is trifluoromethyl; and R¹ is chloro.

In some embodiments, at least one R⁶ is C₁-C₆ alkyl, and at least one R¹ is cyano.

In some embodiments, at least one R⁶ is isopropyl and at least one R¹ is cyano.

In some embodiments, o=1; p=¹; R⁶ is isopropyl; and R¹ is cyano.

In some embodiments, o=2; p=1; at least one R⁶ is isopropyl; and R¹ is cyano.

In some embodiments, at least one R⁶ is C₃-C₇ cycloalkyl, and at least one R¹ is C₃-C₇ cycloalkyl.

In some embodiments, at least one R⁶ is cyclopropyl, and at least one R⁷ is cyclopropyl.

In some embodiments, at least one R⁶ is C₃-C₇ cycloalkyl, and at least one R¹ is halo.

In some embodiments, at least one R⁶ is cyclopropyl and at least one R¹ is halo.

In some embodiments, at least one R⁶ is cyclopropyl and at least one R⁷ is chloro.

In some embodiments, at least one R⁶ is cyclopropyl and at least one R¹ is fluoro.

In some embodiments, o=1; p=¹; R⁶ is cyclopropyl; and R⁷ is chloro.

In some embodiments, o=1; p=1; R⁶ is cyclopropyl; and R⁷ is fluoro.

In some embodiments, at least one R⁶ is C₁-C₆ alkyl, and at least one R¹ is C₁-C₆ alkoxy optionally substituted with one or more halo.

In some embodiments, at least one R⁶ is isopropyl, and at least one R¹ is C₁-C₆ alkoxy.

In some embodiments, at least one R⁶ is isopropyl, and at least one R⁷ is methoxy.

In some embodiments, o=1; p=1; R⁶ is isopropyl, and R¹ is methoxy.

In some embodiments, o=2; p=1; at least one R⁶ is isopropyl, and R¹ is methoxy.

In some embodiments, at least one R⁶ is C₁-C₆ alkyl, and at least one R¹ is C₁-C₆ alkoxy substituted with one or more halo.

In some embodiments, at least one R⁶ is isopropyl, and at least one R¹ is trifluoromethoxy.

In some embodiments, at least one R⁶ is isopropyl, and at least one R¹ is difluoromethoxy.

In some embodiments, at least one R⁶ is C₁-C₆ alkyl, and at least one R¹ is C₁-C₆ alkyl (e.g., methyl) substituted with one or more (e.g., one) C₁-C₆ alkoxy (e.g., methoxy).

In some embodiments, at least on R⁶ is isopropyl, and at least one R⁷ is methoxymethyl.

In some embodiments, o=2; p=1, each R⁶ is isopropyl, and R⁷ is methoxymethyl.

In some embodiments, at least one R⁶ is halo, and at least one R¹ is C₁-C₆ haloalkyl optionally substituted with hydroxy.

In some embodiments, o=1; p=1; R⁶ is chloro, and R⁷ is trifluoromethyl.

In some embodiments, at least one R⁶ is halo, and at least one R¹ is C₁-C₆ haloalkoxy.

In some embodiments, at least one R⁶ is chloro, and at least one R¹ is trifluoromethoxy.

In some embodiments, o=1; p=1; R⁶ is chloro, and R¹ is trifluoromethoxy.

In some embodiments, at least one R⁶ is C₁-C₆ alkoxy; and at least one R⁷ is halo.

In some embodiments, o=1; p=²; R⁶ is C₁-C₆ alkoxy; and at least one R⁷ is chloro.

In some embodiments, at least one R⁶ is C₃-C₇cycloalkyl; and at least one R¹ is C₁-C₆ haloalkyl optionally substituted with hydroxy.

In some embodiments, at least one R⁶ is cyclopropyl; and at least one R⁷ is trifluoromethyl.

In some embodiments, o=1; p=2; R⁶ is cyclopropyl; one R⁷ is trifluoromethyl; and the other R⁷ is fluoro.

In some embodiments, at least one R⁶ is C₁-C₆ alkyl, and at least one R¹ is C₆-C₁₀ aryl, wherein the C₆-C₁₀ aryl is optionally substituted or optionally fused as described elsewhere herein.

In some embodiments, at least one R⁶ is isopropyl, and at least one R¹ is C₆-C₁₀ aryl, wherein the C₆-C₁₀ aryl is optionally substituted as described elsewhere herein.

In some embodiments, at least one R⁶ is isopropyl, and at least one R¹ is dichlorophenyl (e.g., 3,4-dichlorophenyl).

In some embodiments, at least one R⁶ is isopropyl, and at least one R⁷ is dimethylphenyl (e.g., 3,4-dimethylphenyl).

In some embodiments, at least one R⁶ is isopropyl, and at least one R⁷ is naphthyl (e.g., napthyl substituted with one methoxy).

In some embodiments, at least one R⁶ is isopropyl, and at least one R⁷ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

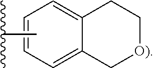

In some embodiments, o=2; p=1, each R⁶ is isopropyl; and R⁷ is C₆-C₁₀ aryl, wherein the C₆-C₁₀ aryl is optionally substituted as described elsewhere herein (e.g., R⁷ is dimethylphenyl; or R⁷ is dichlorophenyl; or R⁷ is naphthyl).

In some embodiments, o=2; p=1, each R⁶ is isopropyl; and R⁷ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

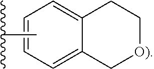

In some embodiments, at least one R⁶ is C₁-C₆ alkyl, and at least one R⁷ is 3- to 7-membered heterocycloalkyl, wherein the 3- to 7-membered heterocycloalkyl is optionally substituted or optionally fused as described elsewhere herein.

In some embodiments, at least one R⁶ is isopropyl, and at least one R⁷ is 3- to 7-membered heterocycloalkyl, wherein the 3- to 7-membered heterocycloalkyl is optionally substituted as described elsewhere herein.

In some embodiments, at least one R⁶ is isopropyl, and at least one R⁷ is tetrahydrofuranyl.

In some embodiments, at least one R⁶ is isopropyl, and at least one R⁷ is

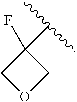 [SMILES: *C1(F)COC1]

In some embodiments, at least one R⁶ is C₁-C₆ alkyl, and at least one R⁷ is 5- to 10-membered heteroaryl, wherein the 5- to 10-membered heteroaryl is optionally substituted or optionally fused as described elsewhere herein.

In some embodiments, at least one R⁶ isopropyl, and at least one R⁷ is 5- to 10-membered heteroaryl, wherein the 5- to 10-membered heteroaryl is optionally substituted or optionally fused as described elsewhere herein.

In some embodiments, at least one R⁶ isopropyl, and at least one R⁷ is pyrazolyl.

In some embodiments, at least one R⁷ is C₁-C₆ alkyl, and at least one R⁶ is C₁-C₆ alkyl optionally substituted with one or more halo.

In some embodiments, at least one R⁷ is isopropyl and at least one R⁶ is methyl.

In some embodiments, at least one R⁷ is C₁-C₆ alkyl, and at least one R⁶ is C₁-C₆ alkyl substituted with one or more halo.

In some embodiments, at least one R⁷ is isopropyl and at least one R⁶ is trifluoromethyl.

In some embodiments, at least one R⁷ is C₁-C₆ alkyl, and at least one R⁶ is C₃-C₇ cycloalkyl.

In some embodiments, at least one R⁷ is isopropyl and at least one R⁶ is cyclopropyl.

In some embodiments, o=1; p=1; R⁷ is isopropyl; and R⁶ is cyclopropyl.

In some embodiments, o=2; p=¹; R⁷ is isopropyl; one R⁶ is cyclopropyl; and the other R⁶ is fluoro.

In some embodiments, o=2; p=2; one R⁷ is isopropyl; one R⁶ is cyclopropyl; the other R⁷ is cyano; and the other R⁶ is fluoro.

In some embodiments, at least one R⁷ is C₁-C₆ alkyl, and at least one R⁶ is halo.

In some embodiments, at least one R⁷ is isopropyl and at least one R⁶ is halo.

In some embodiments, at least one R⁷ is isopropyl and at least one R⁶ is chloro.

In some embodiments, at least one R⁷ is isopropyl and at least one R⁶ is fluoro.

In some embodiments, o=1; p=1; R⁷ is isopropyl; and R⁶ is chloro.

In some embodiments, o=2; p=1; at least one R⁷ is isopropyl; and at least one R⁶ is chloro.

In some embodiments, o=1; p=1; R⁷ is isopropyl; and R⁶ is fluoro.

In some embodiments, o=2; p=1; R⁷ is isopropyl; and at least one R⁶ is fluoro.

In some embodiments, o=1; p=2; each R⁷ is isopropyl, and R⁶ is fluoro.

In some embodiments, o=2; p=2; at least one R⁷ is isopropyl; and at least one R⁶ is fluoro.

In some embodiments, o=2; p=2; at least one R⁷ is isopropyl; one R⁶ is fluoro; and the other R⁶ is cyano.

In some embodiments, o=2; p=1; R⁷ is ethyl; and at least one R⁶ is fluoro.

In some embodiments, o=1; p=²; one R⁷ is isopropyl; the other R⁷ is trifluoromethyl; and R⁶ is chloro.

In some embodiments, at least one R⁷ is C₁-C₆ alkyl, and at least one R⁶ is cyano.

In some embodiments, at least one R⁷ is isopropyl and at least one R⁶ is cyano.

In some embodiments, o=1; p=1; R⁷ is isopropyl; and R⁶ is cyano.

In some embodiments, o=2; p=¹; R⁷ is isopropyl; and at least one R⁶ is cyano.

In some embodiments, at least one R⁷ is C₃-C₇ cycloalkyl, and at least one R⁶ is C₃-C₇ cycloalkyl.

In some embodiments, at least one R⁷ is cyclopropyl, and at least one R⁶ is cyclopropyl.

In some embodiments, at least one R⁷ is C₃-C₇ cycloalkyl, and at least one R⁶ is halo.

In some embodiments, at least one R⁷ is cyclopropyl and at least one R⁶ is halo.

In some embodiments, at least one R⁷ is cyclopropyl and at least one R⁶ is chloro.

In some embodiments, at least one R⁷ is cyclopropyl and at least one R⁶ is fluoro.

In some embodiments, o=1; p=1; R⁷ is cyclopropyl; and R⁶ is chloro.

In some embodiments, o=1; p=¹; R⁷ is cyclopropyl; and R⁶ is fluoro.

In some embodiments, at least one R⁷ is C₁-C₆ alkyl, and at least one R⁶ is C₁-C₆ alkoxy optionally substituted with one or more halo.

In some embodiments, at least one R⁷ is isopropyl, and at least one R⁶ is C₁-C₆ alkoxy.

In some embodiments, at least one R⁷ is isopropyl, and at least one R⁶ is methoxy.

In some embodiments, o=1; p=1; R⁷ is isopropyl, and R⁶ is methoxy.

In some embodiments, o=2; p=1; R⁷ is isopropyl, and at least one R⁶ is methoxy.

In some embodiments, at least one R⁷ is C₁-C₆ alkyl, and at least one R⁶ is C₁-C₆ alkoxy substituted with one or more halo.

In some embodiments, at least one R⁷ is isopropyl, and at least one R⁶ is trifluoromethoxy.

In some embodiments, at least one R⁷ is C₁-C₆ alkyl, and at least one R⁶ is C₁-C₆ alkyl (e.g., methyl) substituted with one or more (e.g., one) C₁-C₆ alkoxy (e.g., methoxy).

In some embodiments, at least on R⁷ is isopropyl, and at least one R⁶ is methoxymethyl.

In some embodiments, o=2; p=1, each R⁷ is isopropyl, and R⁶ is methoxymethyl.

In some embodiments, at least one R⁷ is halo, and at least one R⁶ is C₁-C₆ haloalkyl optionally substituted with hydroxy.

In some embodiments, o=1; p=1; R⁷ is chloro, and R⁶ is trifluoromethyl.

In some embodiments, at least one R¹ is halo, and at least one R⁶ is C₁-C₆ haloalkoxy.

In some embodiments, at least one R⁷ is chloro, and at least one R⁶ is trifluoromethoxy.

In some embodiments, o=1; p=1; R¹ is chloro, and R⁶ is trifluoromethoxy.

In some embodiments, at least one R¹ is C₁-C₆ alkoxy; and at least one R⁶ is halo.

In some embodiments, o=1; p=²; at least one R¹ is C₁-C₆ alkoxy; and R⁶ is chloro.

In some embodiments, at least one R¹ is C₃-C₇ cycloalkyl; and at least one R⁶ is C₁-C₆ haloalkyl optionally substituted with hydroxy.

In some embodiments, at least one R¹ is cyclopropyl; and at least one R⁶ is trifluoromethyl.

In some embodiments, o=2; p=1; R⁷ is cyclopropyl; one R⁶ is trifluoromethyl; and the other R⁶ is fluoro.

In some embodiments, at least one R⁶ is C₁-C₆ alkyl, and at least one R¹ is C₆-C₁₀ aryl, wherein the C₆-C₁₀ aryl is optionally substituted or optionally fused as described elsewhere herein.

In some embodiments, at least one R⁶ is isopropyl, and at least one R¹ is C₆-C₁₀ aryl, wherein the C₆-C₁₀ aryl is optionally substituted as described elsewhere herein.

In some embodiments, at least one R⁶ is isopropyl, and at least one R¹ is dichlorophenyl (e.g., 3,4-dichlorophenyl).

In some embodiments, at least one R⁶ is isopropyl, and at least one R¹ is dimethylphenyl (e.g., 3,4-dimethylphenyl).

In some embodiments, at least one R⁶ is isopropyl, and at least one R⁷ is naphthyl (e.g., napthyl substituted with one methoxy).

In some embodiments, at least one R is isopropyl, and at least one R⁶ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

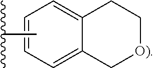

In some embodiments, o=2; p=1, each R¹ is isopropyl; and R⁶ is C₆-C₁₀ aryl, wherein the C₆-C₁₀ aryl is optionally substituted as described elsewhere herein (e.g., R⁶ is dimethylphenyl; or R⁶ is dichlorophenyl; or R⁶ is naphthyl).

In some embodiments, o=2; p=1, each R⁷ is isopropyl; and R⁶ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

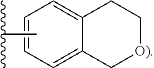

In some embodiments, at least one R⁷ is C₁-C₆ alkyl, and at least one R⁶ is 3- to 7-membered heterocycloalkyl, wherein the 3- to 7-membered heterocycloalkyl is optionally substituted or optionally fused as described elsewhere herein.

In some embodiments, at least one R⁷ is isopropyl, and at least one R⁶ is 3- to 7-membered heterocycloalkyl, wherein the 3- to 7-membered heterocycloalkyl is optionally substituted as described elsewhere herein.

In some embodiments, at least one R⁷ is isopropyl, and at least one R⁶ is tetrahdrofuranyl.

In some embodiments, at least one R⁷ is isopropyl, and at least one R⁶ is

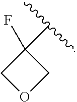 [SMILES: *C1(F)COC1]

In some embodiments, at least one R⁷ is C₁-C₆ alkyl, and at least one R⁶ is 5- to 10-membered heteroaryl, wherein the 5- to 10-membered heteroaryl is optionally substituted or optionally fused as described elsewhere herein.

In some embodiments, at least one R⁷ isopropyl, and at least one R⁶ is 5- to 10-membered heteroaryl, wherein the 5- to 10-membered heteroaryl is optionally substituted or optionally fused as described elsewhere herein.

In some embodiments, at least one R⁷ isopropyl, and at least one R⁶ is pyrazolyl.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, one R⁶ and one R⁷ are on adjacent atoms, and taken together with the atoms connecting them, form a C₈ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₈ aliphatic carbocyclic ring.

In some embodiments, R⁶ and R⁷ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₆ aliphatic carbocyclic ring.

In some embodiments, R⁶ and R⁷ are on adjacent atoms, and taken together with the atoms connecting them, form a C₆ aromatic carbocyclic ring.

In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 5-membered aliphatic heterocyclic ring containing 1 or 2 heteroatoms independently selected from O, N, and S.

In some embodiments, R⁶ and R⁷ are on adjacent atoms, and taken together with the atoms connecting them, form a 5-membered heteroaromatic ring containing 1 or 2 heteroatoms independently selected from O, N, and S.

In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 6-membered aliphatic heterocyclic ring containing 1 or 2 heteroatoms independently selected from O, N, and S.

In some embodiments, R⁶ and R⁷ are on adjacent atoms, and taken together with the atoms connecting them, form a 6-membered heteroaromatic ring containing 1 or 2 heteroatoms independently selected from O, N, and S.

In some embodiments, one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S,
wherein the ring is fused to the B ring at the ortho- and meta-positions relative to the bond connecting the B ring to the C(R⁴R⁵) group.

In some embodiments, one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S,
wherein the ring is fused to the B ring at the meta- and para-positions relative to the bond connecting the B ring to the C(R⁴R⁵) group.

In some embodiments, o=2; p=2 or 3; and
two pairs, each of one R⁶ and one R⁷, are on adjacent atoms, and each pair of one R⁶ and one R⁷ taken together with the atoms connecting them form a C₈ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them form a C₈ aliphatic carbocyclic ring.

In some embodiments, o=2; p=2 or 3; and
two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them form a C₆ aliphatic carbocyclic ring.

In some embodiments, o=2; p=2 or 3; and
two pairs, each of one R⁶ and one R⁷, are on adjacent atoms, and each pair of one R⁶ and one R⁷ taken together with the atoms connecting them form a C₆ aromatic carbocyclic ring.

In some embodiments, o=2; p=2 or 3; and
two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and
two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S.

In some embodiments, o=2; p=2 or 3; and
two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and
two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S.

In some embodiments, o=2; p=2 or 3; and
two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S.

In some embodiments, o=2; p=2 or 3; and
two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S,
wherein one of the two rings is fused to the B ring at the 2- and 3-positions relative to the bond connecting the B ring to the C(R⁴R⁵) group, and the other of the two rings is fused to the B ring at the 5- and 6-positions relative to the bond connecting the B ring to the C(R⁴R⁵) group.

In some embodiments, o=2; p=2; and
two pairs, each of one R⁶ and one R⁷, are on adjacent atoms, and each pair of one R⁶ and one R⁷ taken together with the atoms connecting them form a C₈ aliphatic carbocyclic ring.

In some embodiments, o=2; p=³; and
two pairs, each of one R⁶ and one R⁷, are on adjacent atoms, and each pair of one R⁶ and one R⁷ taken together with the atoms connecting them form a C₈ aliphatic carbocyclic ring; and one R⁷ is halo (e.g., Cl or F).

In some embodiments, o=2; p=³; and
two pairs, each of one R⁶ and one R⁷, are on adjacent atoms, and each pair of one R⁶ and one R⁷ taken together with the atoms connecting them form a C₈ aliphatic carbocyclic ring; and one R⁷ is CN.

In some embodiments, one R⁷ is pyrazolyl and is para to the bond connecting the B ring to the C(R⁴R³) group of Formula AA.

In some embodiments, one R⁷ is 3-pyrazolyl and is para to the bond connecting the B ring to the C(R⁴R⁵) group of Formula AA.

In some embodiments, one R⁷ is 4-pyrazolyl and is para to the bond connecting the B ring to the C(R⁴R⁵) group of Formula AA.

In some embodiments, one R⁷ is 5-pyrazolyl and is para to the bond connecting the B ring to the C(R⁴R⁵) group of Formula AA.

In some embodiments, one R⁷ is thiazolyl and is para to the bond connecting the B ring to the C(R⁴R⁵) group of Formula AA.

In some embodiments, one R⁷ is 4-thiazolyl and is para to the bond connecting the B ring to the C(R⁴R⁵) group of Formula AA.

In some embodiments, one R⁷ is 5-thiazolyl and is para to the bond connecting the B ring to the C(R⁴R⁵) group of Formula AA.

In some embodiments, one R⁷ is furyl and is para to the bond connecting the B ring to the C(R⁴R⁵) group of Formula AA.

In some embodiments, one R⁷ is 2-furyl and is para to the bond connecting the B ring to the C(R⁴R³) group of Formula AA.

In some embodiments, one R⁷ is thiophenyl and is para to the bond connecting the B ring to the C(R⁴R⁵) group of Formula AA.

In some embodiments, one R⁷ is 2-thiophenyl and is para to the bond connecting the B ring to the C(R⁴R⁵) group of Formula AA.

In some embodiments, one R⁷ is phenyl and is para to the bond connecting the B ring to the C(R⁴R⁵) group of Formula AA.

In some embodiments, one R⁷ is naphthyl (e.g., unsubstituted naphthyl or methoxynaphthyl) and is para to the bond connecting the B ring to the C(R⁴R⁵) group of Formula AA.

In some embodiments, one R⁷ is isochromanyl and is para to the bond connecting the B ring to the C(R⁴R⁵) group of Formula AA.

In some embodiments, one R⁷ is cycloalkenyl (e.g., cyclopentenyl, e.g., 1-cyclopentenyl) and is para to the bond connecting the B ring to the C(R⁴R⁵) group of Formula AA.

In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the C(R⁴R⁵) group of Formula AA.

In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the C(R⁴R⁵) group of Formula AA.

In some embodiments, one R⁷ is phenyl optionally substituted with one or more C₆-C₁₀ aryloxy (e.g., phenoxy) and is para to the bond connecting the B ring to the C(R⁴R⁵) group of Formula AA.

In some embodiments, one R¹ is phenyl optionally substituted with one or more CN and is para to the bond connecting the B ring to the C(R⁴R⁵) group of Formula AA.

In some embodiments, one R⁷ is phenyl optionally substituted with one or more halo (e.g., F, Cl) and is para to the bond connecting the B ring to the C(R⁴R⁵) group of Formula AA.

In some embodiments, one R¹ is phenyl optionally substituted with one or more COOC₁-C₆ alkyl (e.g., CO₂t-Bu) and is para to the bond connecting the B ring to the C(R⁴R⁵) group of Formula AA.

In some embodiments, one R⁷ is phenyl optionally substituted with one or more S(O₂)C₁-C₆ alkyl (e.g., S(O₂)methyl) and is para to the bond connecting the B ring to the C(R⁴R⁵) group of Formula AA.

In some embodiments, one R¹ is phenyl optionally substituted with one or more 3- to 7-membered heterocycloalkyl (e.g., morpholinyl) and is para to the bond connecting the B ring to the C(R⁴R⁵) group of Formula AA.

In some embodiments, one R¹ is phenyl optionally substituted with one or more CONR⁸R⁹ (e.g., unsubstituted amido) and is para to the bond connecting the B ring to the C(R⁴R⁵) group of Formula AA.

In some embodiments, one R⁷ is phenyl optionally substituted with one or more C₁-C₆ alkyl (e.g., methyl or propyl, e.g., 2-propyl) and with one or more halo (e.g., F, Cl) and is para to the bond connecting the B ring to the C(R⁴R⁵) group of Formula AA.

In some embodiments, one R¹ is phenyl optionally substituted with one or more C₁-C₆ alkyl (e.g., methyl or propyl, e.g., 2-propyl) optionally substituted with one or more (e.g., one) halo (e.g., fluoro) and is para to the bond connecting the B ring to the C(R⁴R⁵) group of Formula AA.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R¹ is attached to a carbon and R⁶ is attached to a nitrogen of a heteroaryl ring B.

In some embodiments, the substituted ring B is

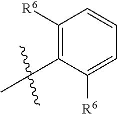

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4-to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the substituted ring B is

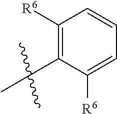

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the substituted ring B is

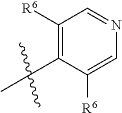

and each R⁶ is independently selected from the group consisting of: C₁-C₆alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR^{IO}, COOC₁-C₆ alkyl, CONR⁸R⁹, 4-to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the substituted ring B is

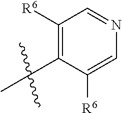

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the substituted ring B is

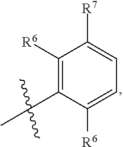

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

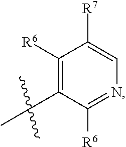

wherein each R⁶ is independently selected from C₁-C₆alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

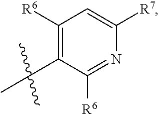

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments, the substituted ring B is

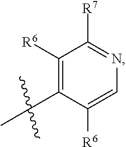

wherein each R⁶ is independently selected from C₁-C₆alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

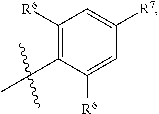

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments, the substituted ring B is

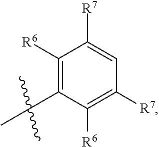

wherein each R⁶ is independently selected from C₁-C₆alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R¹ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

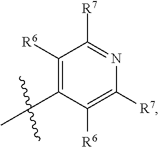

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S,
wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

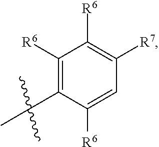

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

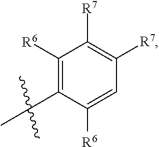

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

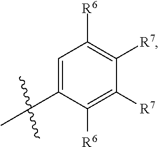

wherein each R⁶ is independently selected from C₁-C₆alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR¹R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the substituted ring B is

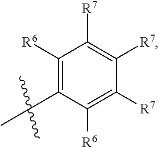

wherein each R⁶ is independently selected from C₁-C₆alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
   wherein each R⁷ is independently selected from C₁-C₆alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.
      The Groups R⁴ and R⁵

In some embodiments, each of R⁴ and R⁵ is independently selected from hydrogen and C₁-C₆ alkyl.

In some embodiments, R⁴ is hydrogen.

In some embodiments, R⁵ is hydrogen.

In some embodiments, each of R⁴ and R⁵ is hydrogen.

In some embodiments, R⁴ is C₁-C₆ alkyl.

In some embodiments, R⁵ is C₁-C₆ alkyl.

In some embodiments, each of R⁴ and R is C₁-C₆ alkyl,

In some embodiments, R⁴ is hydrogen and R⁵ is C₁-C₆ alkyl.

In some embodiments, R⁴ is hydrogen; R⁵ is C₁-C₆ alkyl; and the carbon bonded to R⁴ and R⁵ has (S) stereochemistry.

In some embodiments, R⁴ is hydrogen; and R⁵ is C₁-C₆ alkyl; and the carbon bonded to R⁴ and has (R) stereochemistry.

### The Group R³

In some embodiments, R³ is selected from hydrogen, C₁-C₆ alkyl, and

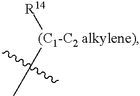

wherein the C₁-C₂ alkylene group is optionally substituted by oxo.

In some embodiments, R³ is hydrogen.

In some embodiments, R³ is cyano.

In some embodiments, R³ is hydroxy.

In some embodiments, R³ is C₁-C₆alkoxy.

In some embodiments, R³ is C₁-C₆ alkyl.

In some embodiments, R³ is methyl.

In some embodiments, R³ is

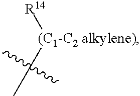

wherein the C₁-C₂ alkylene group is optionally substituted by oxo.

In some embodiments, R³ is —CH₂R¹⁴.

In some embodiments, R³ is —C(O)R¹⁴.

In some embodiments, R³ is —CH₂CH₂R¹⁴.

In some embodiments, R³ is —CHR¹⁴CH₃.

In some embodiments, R³ is —CH₂C(O)R¹⁴.

In some embodiments, R³ is —C(O)CH₂R¹⁴.

### The Group R⁴

In some embodiments, R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen or C₁-C₆ alkyl.

In some embodiments, R¹⁴ is hydrogen, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen.

In some embodiments, R¹⁴ is C₁-C₆ alkyl.

In some embodiments, R¹⁴ is methyl.

In some embodiments, R¹⁴ is 5-10-membered monocyclic or bicyclic heteroaryl optionally independently substituted with 1 or 2 R⁶.

In some embodiments, R¹⁴ is C₆-C₁₀ monocyclic or bicyclic aryl optionally independently substituted with 1 or 2 R⁶.

The moiety S(═O)(NHR³)═N—

In some embodiments, the sulfur in the moiety S(═O)(NHR³))=N— has (S) stereochemistry.

In some embodiments, the sulfur in the moiety S(═O)(NHR³)═N— has (R) stereochemistry.

### The Group R¹⁰

In some embodiments, R¹⁰ is C₁-C₆ alkyl.

In some embodiments, R¹⁰ is methyl.

In some embodiments, R¹⁰ is ethyl.

The Groups R⁸ and R⁹

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is hydrogen,

In some embodiments, each R⁸ at each occurrence is hydrogen and each R¹ at each occurrence is C₁-C₆ alkyl.

In some embodiments, each R^{x} at each occurrence is hydrogen and each R⁹ at each occurrence is methyl.

In some embodiments, each R⁸ at each occurrence is hydrogen and each R¹ at each occurrence is ethyl.

In some embodiments, each of R⁸ and R⁹ at each occurrence is methyl.

In some embodiments, each of R⁸ and R⁹ at each occurrence is ethyl.

In some embodiments, R⁸ and R⁹ taken together with the nitrogen they are attached to form a 3-membered ring.

In some embodiments, R⁸ and R⁹ taken together with the nitrogen they are attached to form a 4-membered ring.

In some embodiments, R⁸ and R⁹ taken together with the nitrogen they are attached to form a 5-membered ring.

In some embodiments, R⁸ and R⁹ taken together with the nitrogen they are attached to form a 6-membered ring optionally containing one or more oxygen atoms in addition to the nitrogen they are attached to.

In some embodiments, R⁸ and R⁹ taken together with the nitrogen they are attached to form a 6-membered ring optionally containing one or more nitrogen atoms in addition to the nitrogen they are attached to.

In some embodiments, R⁸ and R⁹ taken together with the nitrogen they are attached to form a 7-membered ring.

### The Group R¹³

In some embodiments, R¹³ is C₁-C₆ alkyl.

In some embodiments, R¹³ is methyl.

In some embodiments, R¹³ is ethyl.

In some embodiments, R¹³ is C₆-C₁₀ aryl.

In some embodiments, R¹³ is phenyl.

In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl.

In some embodiments, each of R¹¹ and R¹² at each occurrence is hydrogen,

In some embodiments, each R¹¹ at each occurrence is hydrogen and each R¹² at each occurrence is C₁-C₆ alkyl.

In some embodiments, each R¹¹ at each occurrence is hydrogen and each R¹² at each occurrence is methyl.

In some embodiments, each R¹¹ at each occurrence is hydrogen and each R¹² at each occurrence is ethyl.

In some embodiments, each of R¹¹ and R¹² at each occurrence is methyl.

In some embodiments, each of R¹¹ and R¹² at each occurrence is ethyl.

In some embodiments of the compound of formula AA,
the substituted ring A is

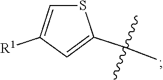

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR^{x}R^{y}; SF₅; and S(O₂)C₁-C₆ alkyl.

In some embodiments of the compound of formula AA,
the substituted ring A is

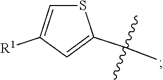

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

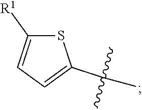

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

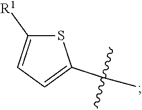

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH—; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

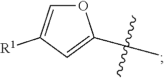

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

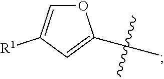

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)C₁—H.

In some embodiments of the compound of formula AA,
the substituted ring A is

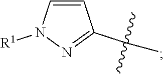

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; and S(O₂)C₁-C₆ alkyl.

In some embodiments of the compound of formula AA,
the substituted ring A is

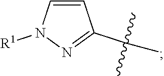

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; phenyl; pyridyl; pyrazolyl; and S(O₂)CH₃.

In some embodiments of the compound of formula AA,
the substituted ring A is

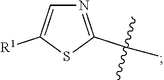

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

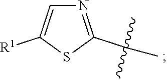

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

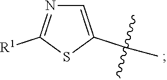

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₅-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

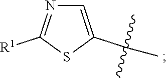

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

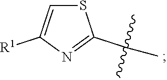

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

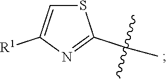

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

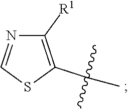

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₈ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

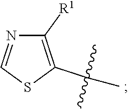

and R¹ is selected from.
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CHM; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

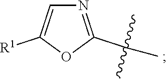

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

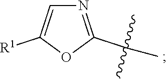

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)C₁—H.

In some embodiments o f formula AA,
the substituted ring A is

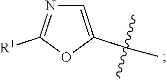

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

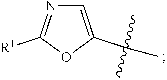

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

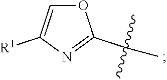

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹); 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

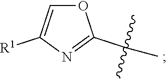

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

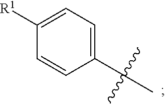

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

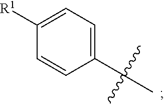

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
the substituted ring A is

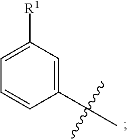

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂, COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

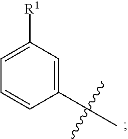

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH.

In some embodiments of the compound of formula AA,
the substituted ring A is

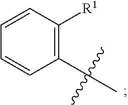

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NI-12; NHC₁-C₆ alkyl; N(C₁-C₆ alkyl)₂; CONR⁸R⁹; SF₅; and S(O₂)C₁-C₆ alkyl.

In some embodiments of the compound of formula AA,
the substituted ring A is

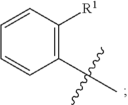

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CHM; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
A is

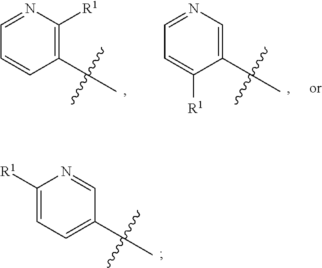

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
A is

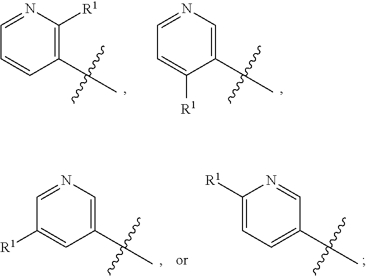

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.
A is

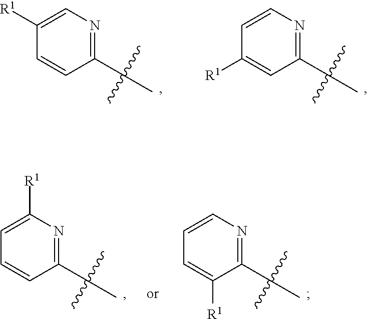

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂, COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
A is

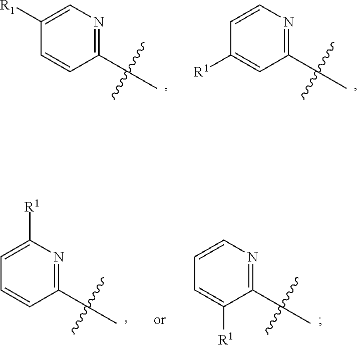

and R¹ is selected from:
   1-hydroxy-2-methylpropan-2-yl; methyl; isopropyl; 2-hydroxy-2-propyl; hydroxymethyl; 1-hydroxyethyl; 2-hydroxy ethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholiny; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.
A is

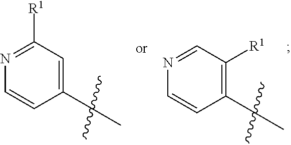

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,
A is

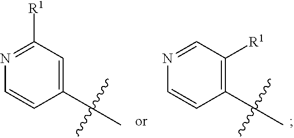

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)C₁—H.

In some embodiments of the compound of formula AA,
A is

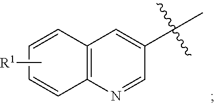

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂, COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered hetero y; NH₂; NHC₁-C₆ alkyl; N(C₁-C₆ alkyl)₂; CONR⁸R⁹; SF₅; and S(O₂)C₁-C₆ alkyl.
A is

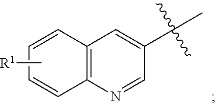

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.
A is

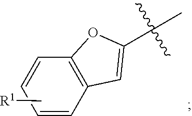

and R¹ is selected from:
   C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; N12; NHC₁-C₆ alkyl; N(C₁-C₆ alkyl)₂; CONR⁸R⁹; SF₅; and S(O₂)C₁-C₆ alkyl.
A is

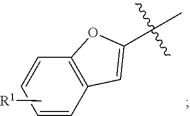

and R¹ is selected from:
   1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,
A is

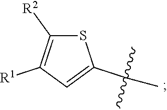

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments of the compound of formula AA,
the substituted ring A is

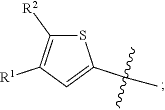

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
(l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

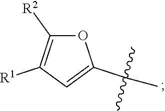

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₇ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments, of the compound of formula AA,
the substituted ring A is

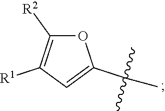

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

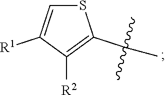

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments, of the compound of formula AA,
the substituted ring A is

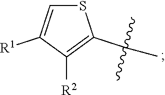

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

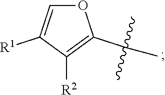

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments, of the compound of formula AA,
the substituted ring A is

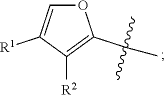

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

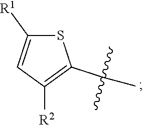

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments, of the compound of formula AA,
the substituted ring A is

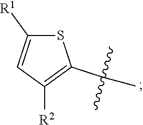

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

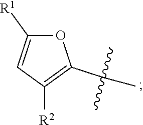

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅;
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
   (xxv) or
   (xxvi) R² is C₁-C₆ alkyl optionally substituted with one or more NR⁸R⁹, and R¹ is halo.

In some embodiments, of the compound of formula AA,
the substituted ring A is

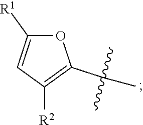

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
   or
      (liv) R² is (dimethylamino)methyl, and R¹ is methyl.

In some embodiments, of the compound of formula AA,
the substituted ring A is

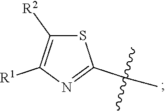

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅;
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

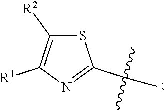

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

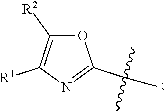

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

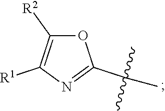

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

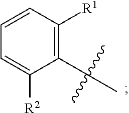

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

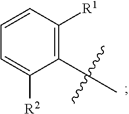

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

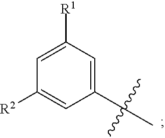

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

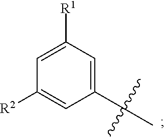

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

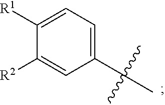

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
   (xxv) R² is C₁-C₆ alkyl optionally substituted with one or more NR⁸R⁹, and R¹ is halo.

In some embodiments, of the compound of formula AA,
the substituted ring A is

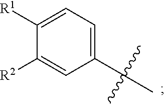

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

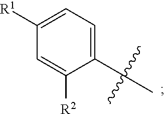

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

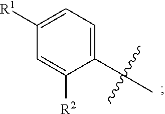

and R and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, of the compound of formula AA,
the substituted ring A is

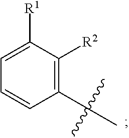

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

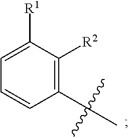

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments, f the compound of formula AA,
the substituted ring A is

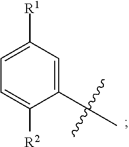

and R¹ and R² are one of the following combinations:
   (i) R¹ is C₁-C₆ alkyl optionally substituted with one or more hydroxy, and R² is C₁-C₆ alkyl optionally substituted with one or more hydroxy;
   (ii) R¹ is C₁-C₆ alkyl optionally substituted with one or more hydroxy, and R² is C₆-C₁₀ aryl;
   (iii) R¹ is C₁-C₆ alkyl optionally substituted with one or more hydroxy, and R² is 5- to 10-membered heteroaryl;
   (iv) R¹ is C₁-C₆ alkyl optionally substituted with one or more hydroxy, and R² is SF₅;
   (v) R¹ is C₁-C₆ alkyl optionally substituted with one or more hydroxy, and R² is S(O₂)C₁-C₆ alkyl;
   (vi) R¹ is C₁-C₆ alkyl optionally substituted with one or more hydroxy, and R² is halo;
   (vii) R¹ is C₃-C₇ cycloalkyl optionally substituted with one or more hydroxy, and R² is C₁-C₆ alkyl;
   (viii) R¹ is 3- to 7-membered heterocycloalkyl optionally substituted with one or more hydroxy, and R² is C₁-C₆ alkyl;
   (ix) R¹ is 3- to 7-membered heterocycloalkyl optionally substituted with one or more hydroxy, and R² is halo;
   (x) R¹ is C₁-C₆ alkyl optionally substituted with one or more oxo, and R² is methyl;
   (xi) R¹ is C₁-C₆ alkyl optionally substituted with one or more C₁-C₆ alkoxy, and R² is C₁-C₆ alkyl;
   (xii) R¹ is C₁-C₆ alkyl optionally substituted with one or more NR⁸R⁹, and R² is C₁-C₆ alkyl;
   (xiii) R¹ is C₁-C₆ alkyl optionally substituted with one or more NR⁸R⁹, and R² is halo;
   (xiv) R² is C₁-C₆ alkyl optionally substituted with one or more hydroxy, and R¹ is C₆-C₁₀ aryl;
   (xv) R² is C₁-C₆ alkyl optionally substituted with one or more hydroxy, and R¹ is 5- to 10-membered heteroaryl;
   (xvi) R² is C₁-C₆ alkyl optionally substituted with one or more hydroxy, and R¹ is SF₅.
   (xvii) R² is C₁-C₆ alkyl optionally substituted with one or more hydroxy, and R¹ is S(O₂)C₁-C₆ alkyl;
   (xviii) R² is C₁-C₆ alkyl optionally substituted with one or more hydroxy, and R¹ is halo;
   (xix) R² is C₃-C₇ cycloalkyl optionally substituted with one or more hydroxy, and R¹ is C₁-C₆ alkyl;
   (xx) R² is 3- to 7-membered heterocycloalkyl optionally substituted with one or more hydroxy, and R¹ is C₁-C₆ alkyl;
   (xxi) R² is 3- to 7-membered heterocycloalkyl optionally substituted with one or more hydroxy, and R¹ is halo;
   (xxii) R² is C₁-C₆ alkyl optionally substituted with one or more oxo, and R¹ is methyl;
   (xxiii) R² is C₁-C₆ alkyl optionally substituted with one or more C₁-C₆ alkoxy, and R¹ is C₁-C₆ alkyl;
   (xxiv) R² is C₁-C₆ alkyl optionally substituted with one or more NR⁸R⁹, and R¹ is C₁-C₆ alkyl;
      or
(xxv) R² is C₁-C₆ alkyl optionally substituted with one or more NR⁸R⁹, and R¹ is halo.

In some embodiments, of the compound of formula AA,
the substituted ring A is

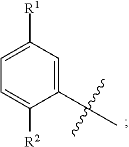

and R¹ and R² are one of the following combinations:
   (i) R¹ is 1-hydroxy-2-methylpropan-2-yl, and R² is methyl;
   (ii) R¹ is 2-hydroxy-2-propyl and R² is methyl;
   (iii) R¹ is 2-hydroxy-2-propyl and R² is isopropyl;
   (iv) R¹ is 2-hydroxy-2-propyl and R² is 2-hydroxy-2-propyl;
   (v) R¹ is 2-hydroxy-2-propyl and R² is 1-hydroxyethyl;
   (vi) R¹ is hydroxymethyl and R² is methyl;
   (vii) R¹ is 1-hydroxyethyl and R² is methyl;
   (viii) R¹ is 2-hydroxyethyl and R² is methyl;
   (ix) R¹ is 1-hydroxy-2-propyl and R² is methyl;
   (x) R¹ is 2-hydroxy-2-propyl and R² is phenyl;
   (xi) R¹ is 2-hydroxy-2-propyl and R² is pyridyl;
   (xii) R¹ is 2-hydroxy-2-propyl and R² is pyrazolyl;
   (xiii) R¹ is 2-hydroxy-2-propyl, and R² is S(O₂)CH₃;
   (xiv) R¹ is 2-hydroxy-2-propyl and R² is chloro;
   (xv) R¹ is 2-hydroxy-2-propyl and R² is fluoro;
   (xvi) R¹ is 1-hydroxy-1-cyclopropyl, and R² is methyl;
   (xvii) R¹ is 1-hydroxy-1-cyclobutyl, and R² is methyl;
   (xviii) R¹ is 1-hydroxy-1-cyclopentyl, and R² is methyl;
   (xix) R¹ is 1-hydroxy-1-cyclohexyl, and R² is methyl;
   (xx) R¹ is morpholinyl, and R² is methyl;
   (xxi) R¹ is 1,3-dioxolan-2-yl, and R² is methyl;
   (xxii) R¹ is 1,3-dioxolan-2-yl, and R² is fluoro;
   (xxiii) R¹ is 1,3-dioxolan-2-yl, and R² is chloro;
   (xxiv) R¹ is COCH₃, and R² is methyl;
   (xxv) R¹ is 2-methoxy-2-propyl, and R² is methyl;
   (xxvi) R¹ is (dimethylamino)methyl, and R² is methyl;
   (xxvii) R² is 1-hydroxy-2-methylpropan-2-yl, and R¹ is methyl;
   (xxviii) R² is 2-hydroxy-2-propyl and R¹ is methyl;
   (xxix) R² is 2-hydroxy-2-propyl and R¹ is isopropyl;
   (xxx) R² is 2-hydroxy-2-propyl and R¹ is 1-hydroxyethyl;
   (xxxi) R² is hydroxymethyl and R¹ is methyl;
   (xxxii) R² is 1-hydroxyethyl and R¹ is methyl;
   (xxxiii) R² is 2-hydroxyethyl and R¹ is methyl;
   (xxxiv) R² is 1-hydroxy-2-propyl and R¹ is methyl;
   (xxxv) R² is 2-hydroxy-2-propyl and R¹ is phenyl;
   (xxxvi) R² is C₁-C₆ alkyl optionally substituted with one or more hydroxy, and R¹ is 5- to 10-membered heteroaryl;
   (xxxvii) R² is 2-hydroxy-2-propyl and R¹ is pyridyl;
   (xxxviii) R² is 2-hydroxy-2-propyl and R¹ is pyrazolyl;
   (xxxix) R² is C₁-C₆ alkyl optionally substituted with one or more hydroxy, and R¹ is S(O₂)CH₃;
   (xl) R² is 2-hydroxy-2-propyl and R¹ is chloro;
   (xli) R² is 2-hydroxy-2-propyl and R¹ is fluoro;
   (xlii) R² is C₃-C₇ cycloalkyl optionally substituted with one or more hydroxy, and R¹ is C₁-C₆ alkyl;
   (xliii) R² is 1-hydroxy-1-cyclopropyl, and R¹ is methyl;
   (xliv) R² is 1-hydroxy-1-cyclobutyl, and R¹ is methyl;
   (xlv) R² is 1-hydroxy-1-cyclopentyl, and R¹ is methyl;
   (xlvi) R² is 1-hydroxy-1-cyclohexyl, and R¹ is methyl;
   (xlvii) R² is morpholinyl, and R¹ is methyl;
   (xlviii) R² is 1,3-dioxolan-2-yl, and R¹ is methyl;
   (xlix) R² is 1,3-dioxolan-2-yl, and R¹ is fluoro;
   (l) R² is 1,3-dioxolan-2-yl, and R¹ is chloro;
   (li) R² is C₁-C₆ alkyl optionally substituted with one or more oxo, and R¹ is methyl;
   (lii) R² is COCH₃, and R¹ is methyl;
   (liii) R² is 2-methoxy-2-propyl, and R¹ is methyl;
      or
   (liv) R² is (dimethylamino)methyl, and R¹ is methyl.

In some embodiments of the compound of formula AA,
the substituted ring B is

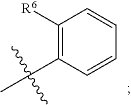

and R⁶ is selected from:
   C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,
the substituted ring B is

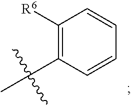

and R⁶ is selected from:
   isopropyl, ethyl, methyl, trifluoromethyl, trifluoromethoxy, cyclopropyl, halo, chloro, and fluoro.

In some embodiments of the compound of formula AA,
the substituted ring B is

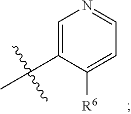

and R⁶ is selected from:
   C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,
the substituted ring B is

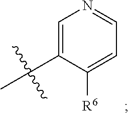

and R⁶ is selected from.
   isopropyl, ethyl, methyl, trifluoromethyl, trifluoromethoxy, cyclopropyl, halo, chloro, and fluoro.

In some embodiments of the compound of formula AA,
the substituted ring B is

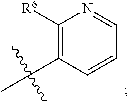

and R⁶ is selected from:
   C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,
the substituted ring B is

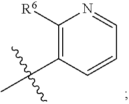

and R⁶ is selected from:
   isopropyl, ethyl, methyl, trifluoromethyl, trifluoromethoxy, cyclopropyl, halo, chloro, and fluoro.

In some embodiments of the compound of formula AA,
the substituted ring B is

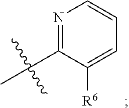

and R⁶ is selected from:
   C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,
the substituted ring B is

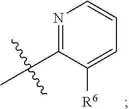

and R⁶ is selected from:
   isopropyl, ethyl, methyl, trifluoromethyl, trifluoromethoxy, cyclopropyl, halo, chloro, and fluoro.

In some embodiments of the compound of formula AA,
the substituted ring B is

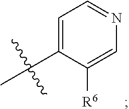

and R⁶ is selected from:
   C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,
the substituted ring B is

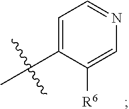

and R⁶ is selected from:
   isopropyl, ethyl, methyl, trifluoromethyl, trifluoromethoxy, cyclopropyl, halo, chloro, and fluoro.

In some embodiments, of the compound of formula AA,
the substituted ring B is

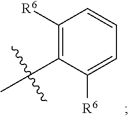

and the two R⁶ are one of the following combinations:
   One R⁶ is C₁-C₆ alkyl, and the other R⁶ is C₁-C₆ alkyl optionally substituted with one or more halo;
   One R⁶ is C₁-C₆ alkyl and the other R⁶ is C₁-C₆ alkyl;
   One R⁶ is C₁-C₆ alkyl, and the other R⁶ is C₁-C₆ alkyl substituted with one or more halo;
   One R⁶ is C₁-C₆ alkyl, and the other R⁶ is C₃-C₇ cycloalkyl;
   One R⁶ is C₁-C₆ alkyl, and the other R⁶ is halo;
   One R⁶ is C₁-C₆ alkyl, and the other R⁶ is cyano;
   One R⁶ is C₃-C₇ cycloalkyl, and the other R⁶ is C₃-C₇ cycloalkyl;
   One R⁶ is C₃-C₇ cycloalkyl, and the other R⁶ is halo;
   One R⁶ is cyclopropyl and the other R⁶ is halo;
   One R⁶ is C₁-C₆ alkyl, and the other R⁶ is C₁-C₆ alkoxy optionally substituted with one or more halo;
   One R⁶ is C₁-C₆ alkyl, and the other R⁶ is C₁-C₆ alkoxy;
   One R⁶ is C₁-C₆ alkyl, and the other R⁶ is C₁-C₆ alkoxy substituted with one or more halo;
   One R⁶ is halo, and the other R⁶ is C₁-C₆ haloalkyl;
   One R⁶ is halo, and the other R⁶ is C₁-C₆ haloalkoxy;
   One R⁶ is C₁-C₆ alkoxy; and the other R⁶ is halo;
   One R⁶ is C₁-C₆ alkoxy; and the other R⁶ is chloro.

In some embodiments, of the compound of formula AA,
the substituted ring B is

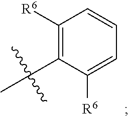

and the two R⁶ are one of the following combinations:
   One R⁶ is isopropyl; and the other R⁶ is methyl;
   One R⁶ is isopropyl; and the other R⁶ is n-propyl;
   One R⁶ is isopropyl; and the other R⁶ is isopropyl;
   One R⁶ is isopropyl; and the other R⁶ is trifluoromethyl;
   One R⁶ is isopropyl; and the other R⁶ is cyclopropyl;
   One R⁶ is isopropyl; and the other R⁶ is chloro;
   One R⁶ is isopropyl; and the other R⁶ is fluoro;
   One R⁶ is ethyl; and the other R⁶ is fluoro;
   One R⁶ is isopropyl; and the other R⁶ is cyano;
   One R⁶ is cyclopropyl; and the other R⁶ is cyclopropyl;
   One R⁶ is cyclopropyl; and the other R⁶ is chloro;
   One R⁶ is cyclopropyl; and the other R⁶ is fluoro;
   One R⁶ is isopropyl; and the other R⁶ is methoxy;
   One R⁶ is isopropyl; and the other R⁶ is trifluoromethoxy.

In some embodiments, of the compound of formula AA,
the substituted ring B is

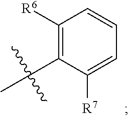

and R⁶ and R⁷ are one of the following combinations:
   R⁶ is C₁-C₆ alkyl, and R⁷ is C₁-C₆ alkyl optionally substituted with one or more halo;
   R⁶ is C₁-C₆ alkyl and R⁷ is C₁-C₆ alkyl;
   R⁶ is C₁-C₆ alkyl, and R⁷ is C₁-C₆ alkyl substituted with one or more halo;
   R⁶ is C₁-C₆ alkyl, and R⁷ is C₃-C₇ cycloalkyl;
   R⁶ is C₁-C₆ alkyl, and R⁷ is halo;
   R⁶ is C₁-C₆ alkyl, and R⁷ is cyano;
   R⁶ is C₃-C₇ cycloalkyl, and R⁷ is C₃-C₇ cycloalkyl;
   R⁶ is C₃-C₇ cycloalkyl, and R⁷ is halo;
   R⁶ is cyclopropyl and R⁷ is halo;
   R⁶ is C₁-C₆ alkyl, and R⁷ is C₁-C₆ alkoxy optionally substituted with one or more halo;
   R⁶ is C₁-C₆ alkyl, and R⁷ is C₁-C₆ alkoxy;
   R⁶ is C₁-C₆ alkyl, and R⁷ is C₁-C₆ alkoxy substituted with one or more halo;
   R⁶ is halo, and R⁷ is C₁-C₆ haloalkyl;
   R⁶ is halo, and R⁷ is C₁-C₆ haloalkoxy;
   R⁶ is C₁-C₆ alkoxy; and R⁷ is halo;
   R⁶ is C₁-C₆ alkoxy; and R⁷ is chloro;
   R⁷ is C₁-C₆ alkyl, and R⁶ is C₁-C₆ alkyl optionally substituted with one or more halo;
   R⁷ is C₁-C₆ alkyl, and R⁶ is C₁-C₆ alkyl substituted with one or more halo;
   R⁷ is C₁-C₆ alkyl, and R⁶ is C₃-C₇ cycloalkyl;
   R⁷ is C₁-C₆ alkyl, and R⁶ is halo;
   R⁷ is C₁-C₆ alkyl and R⁶ is halo;
   R⁷ is C₁-C₆ alkyl, and R⁶ is cyano;
   R⁷ is C₃-C₇ cycloalkyl, and R⁶ is C₃-C₇ cycloalkyl;
   R⁷ is C₃-C₇ cycloalkyl, and R⁶ is halo;
   R⁷ is C₃-C₇ cycloalkyl and R⁶ is halo;
   R⁷ is C₁-C₆ alkyl, and R⁶ is C₁-C₆ alkoxy optionally substituted with one or more halo;
   R⁷ is C₁-C₆ alkyl, and R⁶ is C₁-C₆ alkoxy;
   R⁷ is C₁-C₆ alkyl, and R⁶ is C₁-C₆ alkoxy substituted with one or more halo;
   R⁷ is halo, and R⁶ is C₁-C₆ haloalkyl;
   R⁷ is halo, and R⁶ is C₁-C₆ haloalkoxy;
   R⁷ is C₁-C₆ alkoxy; and R⁶ is halo;
   R⁷ is C₁-C₆ alkoxy; and R⁶ is chloro;

In some embodiments, of the compound of formula AA,
the substituted ring B is R

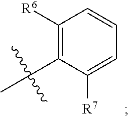

and R⁶ and R⁷ are one of the following combinations:
   R⁶ is isopropyl; and R⁷ is methyl;
   R⁶ is isopropyl; and R⁷ is isopropyl;
   R⁶ is isopropyl; and R⁷ is trifluoromethyl;
   R⁶ is isopropyl; and R⁷ is cyclopropyl;
   R⁶ is isopropyl; and R⁷ is chloro;
   R⁶ is isopropyl; and R⁷ is fluoro;
   R⁶ is ethyl, and R⁷ is fluoro;
   R⁶ is isopropyl; and R⁷ is cyano;
   R⁶ is cyclopropyl; and R⁷ is cyclopropyl;
   R⁶ is cyclopropyl; and R⁷ is chloro;
   R⁶ is cyclopropyl; and R⁷ is fluoro;
   R⁶ is isopropyl; and R⁷ is methoxy;
   R⁶ is isopropyl; and R⁷ is trifluoromethoxy;
   R⁶ is chloro; and R⁷ is trifluoromethyl;
   R⁶ is chloro; and R⁷ is trifluoromethoxy;
   R⁷ is isopropyl; and R⁶ is methyl;
   R⁷ is isopropyl; and R⁶ is trifluoromethyl;
   R⁷ is isopropyl; and R⁶ is cyclopropyl;
   R⁷ is isopropyl; and R⁶ is chloro;
   R⁷ is ethyl; and R⁶ is fluoro;
   R⁷ is isopropyl; and R⁶ is cyano;
   R⁷ is cyclopropyl; and R⁶ is cyclopropyl;
   R⁷ is cyclopropyl; and R⁶ is chloro;
   R⁷ is cyclopropyl; and R⁶ is fluoro;
   R⁷ is isopropyl; and R⁶ is methoxy;
   R⁷ is isopropyl; and R⁶ is trifluoromethoxy;
   R⁷ is chloro; and R⁶ is trifluoromethyl;
   R⁷ is chloro; and R⁶ is trifluoromethoxy.

In some embodiments, of the compound of formula AA,
the substituted ring B is

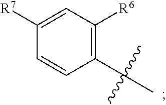

and R⁶ and R⁷ are one of the following combinations:
   R⁶ is C₁-C₆ alkyl, and R⁷ is C₁-C₆ alkyl optionally substituted with one or more halo;
   R⁶ is C₁-C₆ alkyl and R⁷ is C₁-C₆ alkyl;
   R⁶ is C₁-C₆ alkyl, and R⁷ is C₁-C₆ alkyl substituted with one or more halo;
   R⁶ is C₁-C₆ alkyl, and R⁷ is C₃-C₇ cycloalkyl;
   R⁶ is C₁-C₆ alkyl, and R⁷ is halo;
   R⁶ is C₁-C₆ alkyl, and R⁷ is cyano;
   R⁶ is C₃-C₇ cycloalkyl, and R⁷ is C₃-C₇ cycloalkyl;
   R⁶ is C₃-C₇ cycloalkyl, and R⁷ is halo;
   R⁶ is cyclopropyl and R⁷ is halo;
   R⁶ is C₁-C₆ alkyl, and R⁷ is C₁-C₆ alkoxy optionally substituted with one or more halo;
   R⁶ is C₁-C₆ alkyl, and R⁷ is C₁-C₆ alkoxy;
   R⁶ is C₁-C₆ alkyl, and R⁷ is C₁-C₆ alkoxy substituted with one or more halo;
   R⁶ is halo, and R⁷ is C₁-C₆ haloalkyl;
   R⁶ is halo, and R⁷ is C₁-C₆ haloalkoxy;
   R⁶ is C₁-C₆ alkoxy; and R⁷ is halo;
   R⁶ is C₁-C₆ alkoxy; and R⁷ is chloro;
   R⁷ is C₁-C₆ alkyl, and R⁶ is C₁-C₆ alkyl optionally substituted with one or more halo;
   R⁷ is C₁-C₆ alkyl, and R⁶ is C₁-C₆ alkyl substituted with one or more halo;
   R⁷ is C₁-C₆ alkyl, and R⁶ is C₃-C₇ cycloalkyl;
   R⁷ is C₁-C₆ alkyl, and R⁶ is halo;
   R⁷ is C₁-C₆ alkyl and R⁶ is halo;
   R⁷ is C₁-C₆ alkyl, and R⁶ is cyano;
   R⁷ is C₃-C₇ cycloalkyl, and R⁶ is C₃-C₇ cycloalkyl;
   R⁷ is C₃-C₇ cycloalkyl, and R⁶ is halo;
   R⁷ is C₃-C₇ cycloalkyl and R⁶ is halo;
   R⁷ is C₁-C₆ alkyl, and R⁶ is C₁-C₆ alkoxy optionally substituted with one or more halo;
   R⁷ is C₁-C₆ alkyl, and R⁶ is C₁-C₆ alkoxy;
   R⁷ is C₁-C₆ alkyl, and R⁶ is C₁-C₆ alkoxy substituted with one or more halo;
   R⁷ is halo, and R⁶ is C₁-C₆ haloalkyl;
   R⁷ is halo, and R⁶ is C₁-C₆ haloalkoxy;
   R⁷ is C₁-C₆ alkoxy; and R⁶ is halo;
   R⁷ is C₁-C₆ alkoxy; and R⁶ is chloro.

In some embodiments, of the compound of formula AA,
the substituted ring B is

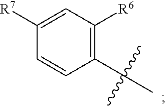

and R⁶ and R⁷ are one of the following combinations:
   R⁶ is isopropyl; and R⁷ is methyl;
   R⁶ is isopropyl; and R⁷ is isopropyl;
   R⁶ is isopropyl; and R⁷ is trifluoromethyl;
   R⁶ is isopropyl; and R⁷ is cyclopropyl;
   R⁶ is isopropyl; and R⁷ is chloro;
   R⁶ is isopropyl; and R⁷ is fluoro;
   R⁶ is ethyl, and R⁷ is fluoro;
   R⁶ is isopropyl; and R⁷ is cyano;
   R⁶ is cyclopropyl; and R⁷ is cyclopropyl;
   R⁶ is cyclopropyl; and R⁷ is chloro;
   R⁶ is cyclopropyl; and R⁷ is fluoro;
   R⁶ is isopropyl; and R⁷ is methoxy;
   R⁶ is isopropyl; and R⁷ is trifluoromethoxy;
   R⁶ is chloro; and R⁷ is trifluoromethyl;
   R⁶ is chloro; and R⁷ is trifluoromethoxy;
   R⁷ is isopropyl; and R⁶ is methyl;
   R⁷ is isopropyl; and R⁶ is trifluoromethyl;
   R⁷ is isopropyl; and R⁶ is cyclopropyl;
   R⁷ is isopropyl; and R⁶ is chloro;
   R⁷ is ethyl, and R⁶ is fluoro;
   R⁷ is isopropyl; and R⁶ is cyano;
   R⁷ is cyclopropyl; and R⁶ is cyclopropyl;
   R⁷ is cyclopropyl; and R⁶ is chloro;
   R⁷ is cyclopropyl; and R⁶ is fluoro;
   R⁷ is isopropyl; and R⁶ is methoxy;
   R⁷ is isopropyl; and R⁶ is trifluoromethoxy;
   R⁷ is chloro; and R⁶ is trifluoromethyl;
   R¹ is chloro; and R⁶ is trifluoromethoxy.

In some embodiments, of the compound of formula AA,
the substituted ring B is

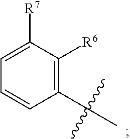

and R⁶ and R⁷ are one of the following combinations:
   R⁶ is C₁-C₆ alkyl, and R⁷ is C₁-C₆ alkyl optionally substituted with one or more halo;
   R⁶ is C₁-C₆ alkyl and R⁷ is C₁-C₆ alkyl;
   R⁶ is C₁-C₆ alkyl, and R⁷ is C₁-C₆ alkyl substituted with one or more halo;
   R⁶ is C₁-C₆ alkyl, and R⁷ is C₃-C₇ cycloalkyl;
   R⁶ is C₁-C₆ alkyl, and R⁷ is halo;
   R⁶ is C₁-C₆ alkyl, and R⁷ is cyano;
   R⁶ is C₃-C₇ cycloalkyl, and R⁷ is C₃-C₇ cycloalkyl;
   R⁶ is C₃-C₇ cycloalkyl, and R⁷ is halo;
   R⁶ is cyclopropyl and R⁷ is halo;
   R⁶ is C₁-C₆ alkyl, and R⁷ is C₁-C₆ alkoxy optionally substituted with one or more halo;
   R⁶ is C₁-C₆ alkyl, and R⁷ is C₁-C₆ alkoxy;
   R⁶ is C₁-C₆ alkyl, and R⁷ is C₁-C₆ alkoxy substituted with one or more halo;
   R⁶ is halo, and R⁷ is C₁-C₆ haloalkyl;
   R⁶ is halo, and R⁷ is C₁-C₆ haloalkoxy;
   R⁶ is C₁-C₆ alkoxy; and R⁷ is halo;
   R⁶ is C₁-C₆ alkoxy; and R⁷ is chloro;
   R⁷ is C₁-C₆ alkyl, and R⁶ is C₁-C₆ alkyl optionally substituted with one or more halo;
   R⁷ is C₁-C₆ alkyl, and R⁶ is C₁-C₆ alkyl substituted with one or more halo;
   R⁷ is C₁-C₆ alkyl, and R⁶ is C₃-C₇ cycloalkyl;
   R⁷ is C₁-C₆ alkyl, and R⁶ is halo;
   R⁷ is C₁-C₆ alkyl and R⁶ is halo;
   R⁷ is C₁-C₆ alkyl, and R⁶ is cyano;
   R⁷ is C₃-C₇ cycloalkyl, and R⁶ is C₃-C₇ cycloalkyl;
   R⁷ is C₃-C₇ cycloalkyl, and R⁶ is halo;
   R⁷ is C₃-C₇ cycloalkyl and R⁶ is halo;
   R⁷ is C₁-C₆ alkyl, and R⁶ is C₁-C₆ alkoxy optionally substituted with one or more halo;
   R⁷ is C₁-C₆ alkyl, and R⁶ is C₁-C₆ alkoxy;
   R⁷ is C₁-C₆ alkyl, and R⁶ is C₁-C₆ alkoxy substituted with one or more halo;
   R⁷ is halo, and R⁶ is C₁-C₆ haloalkyl;
   R⁷ is halo, and R⁶ is C₁-C₆ haloalkoxy;
   R⁷ is C₁-C₆ alkoxy; and R⁶ is halo;
   R⁷ is C₁-C₆ alkoxy; and R⁶ is chloro R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,
the substituted ring B is

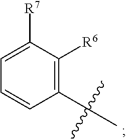

and R⁶ and R⁷ are one of the following combinations:
   R⁶ is isopropyl; and R⁷ is methyl;
   R⁶ is isopropyl; and R⁷ is isopropyl;
   R⁶ is isopropyl; and R⁷ is trifluoromethyl;
   R⁶ is isopropyl; and R⁷ is cyclopropyl;
   R⁶ is isopropyl; and R⁷ is chloro;
   R⁶ is isopropyl; and R⁷ is fluoro;
   R⁶ is ethyl; and R⁷ is fluoro;
   R⁶ is isopropyl; and R⁷ is cyano;
   R⁶ is cyclopropyl; and R⁷ is cyclopropyl;
   R⁶ is cyclopropyl; and R⁷ is chloro;
   R⁶ is cyclopropyl; and R⁷ is fluoro;
   R⁶ is isopropyl; and R⁷ is methoxy;
   R⁶ is isopropyl; and R⁷ is trifluoromethoxy;
   R⁶ is chloro; and R⁷ is trifluoromethyl;
   R⁶ is chloro; and R⁷ is trifluoromethoxy;
   R⁷ is isopropyl; and R⁶ is methyl;
   R⁷ is isopropyl; and R⁶ is trifluoromethyl;
   R⁷ is isopropyl; and R⁶ is cyclopropyl;
   R⁷ is isopropyl; and R⁶ is chloro;
   R⁷ is ethyl; and R⁶ is fluoro;
   R⁷ is isopropyl; and R⁶ is cyano;
   R⁷ is cyclopropyl; and R⁶ is cyclopropyl;
   R⁷ is cyclopropyl; and R⁷ is chloro;
   R⁷ is cyclopropyl; and R⁶ is fluoro;
   R⁷ is isopropyl; and R⁶ is methoxy;
   R⁷ is isopropyl; and R⁶ is trifluoromethoxy;
   R⁷ is chloro; and R⁶ is trifluoromethyl;
   R⁷ is chloro; and R⁷ is trifluoromethoxy;
   R⁶ and R⁷ on adjacent atoms taken together with the atoms connecting them form a C₄ aliphatic carbocyclic ring;
   R⁶ and R⁷ on adjacent atoms taken together with the atoms connecting them form a C₅ aliphatic carbocyclic ring;
   R⁶ and R⁷ on adjacent atoms taken together with the atoms connecting them form a C₆ aliphatic carbocyclic ring;
   R⁶ and R⁷ on adjacent atoms taken together with the atoms connecting them form a 5-membered heterocyclic ring containing 1 heteroatoms independently selected from O, N, and S;
   R⁶ and R⁷ on adjacent atoms taken together with the atoms connecting them form a 6-membered heterocyclic ring containing 1 heteroatoms independently selected from O, N, and S; or
   R⁶ and R⁷ on adjacent atoms taken together with the atoms connecting them form a C₅ aliphatic carbocyclic ring.

In some embodiments, of the compound of formula AA,
the substituted ring B is

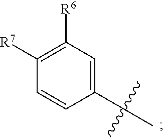

and R⁶ and R⁷ are one of the following combinations:
   R⁶ is C₁-C₆ alkyl, and R⁷ is C₁-C₆ alkyl optionally substituted with one or more halo;
   R⁶ is C₁-C₆ alkyl and R⁷ is C₁-C₆ alkyl;
   R⁶ is C₁-C₆ alkyl, and R⁷ is C₁-C₆ alkyl substituted with one or more halo;
   R⁶ is C₁-C₆ alkyl, and R⁷ is C₃-C₇ cycloalkyl;
   R⁶ is C₁-C₆ alkyl, and R⁷ is halo;
   R⁶ is C₁-C₆ alkyl, and R⁷ is cyano;
   R⁶ is C₃-C₇ cycloalkyl, and R⁷ is C₃-C₇ cycloalkyl;
   R⁶ is C₃-C₇ cycloalkyl, and R⁷ is halo;
   R⁶ is cyclopropyl and R⁷ is halo;
   R⁶ is C₁-C₆ alkyl, and R⁷ is C₁-C₆ alkoxy optionally substituted with one or more halo;
   R⁶ is C₁-C₆ alkyl, and R⁷ is C₁-C₆ alkoxy;
   R⁶ is C₁-C₆ alkyl, and R⁷ is C₁-C₆ alkoxy substituted with one or more halo;
   R⁶ is halo, and R⁷ is C₁-C₆ haloalkyl;
   R⁶ is halo, and R⁷ is C₁-C₆ haloalkoxy;
   R⁶ is C₁-C₆ alkoxy; and R⁷ is halo;
   R⁶ is C₁-C₆ alkoxy; and R⁷ is chloro;
   R⁷ is C₁-C₆ alkyl, and R⁶ is C₁-C₆ alkyl optionally substituted with one or more halo;
   R⁷ is C₁-C₆ alkyl, and R⁶ is C₁-C₆ alkyl substituted with one or more halo;
   R⁷ is C₁-C₆ alkyl, and R⁶ is C₃-C₇ cycloalkyl;
   R⁷ is C₁-C₆ alkyl, and R⁶ is halo;
   R⁷ is C₁-C₆ alkyl and R⁶ is halo;
   R⁷ is C₁-C₆ alkyl, and R⁶ is cyano;
   R⁷ is C₃-C₇ cycloalkyl, and R⁶ is C₃-C₇ cycloalkyl;
   R⁷ is C₃-C₇ cycloalkyl, and R⁶ is halo;
   R⁷ is C₃-C₇ cycloalkyl and R⁶ is halo;
   R⁷ is C₁-C₆ alkyl, and R⁶ is C₁-C₆ alkoxy optionally substituted with one or more halo;
   R⁷ is C₁-C₆ alkyl, and R⁶ is C₁-C₆ alkoxy;
   R⁷ is C₁-C₆ alkyl, and R⁶ is C₁-C₆ alkoxy substituted with one or more halo;
   R⁷ is halo, and R⁶ is C₁-C₆ haloalkyl;
   R⁷ is halo, and R⁶ is C₁-C₆ haloalkoxy;
   R⁷ is C₁-C₆ alkoxy; and R⁶ is halo;
   R⁷ is C₁-C₆ alkoxy; and R⁶ is chloro;
   R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,
the substituted ring B is

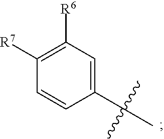

and R⁶ and R⁷ are one of the following combinations:
   R⁶ is isopropyl; and R⁷ is methyl;
   R⁶ is isopropyl; and R⁷ is isopropyl;
   R⁶ is isopropyl; and R⁷ is trifluoromethyl;
   R⁶ is isopropyl; and R⁷ is cyclopropyl;
   R⁶ is isopropyl; and R⁷ is chloro;
   R⁶ is isopropyl; and R⁷ is fluoro;
   R⁶ is ethyl; and R⁷ is fluoro;
   R⁶ is isopropyl; and R⁷ is cyano;
   R⁶ is cyclopropyl; and R⁷ is cyclopropyl;
   R⁶ is cyclopropyl; and R⁷ is chloro;
   R⁶ is cyclopropyl; and R⁷ is fluoro;
   R⁶ is isopropyl; and R⁷ is methoxy;
   R⁶ is isopropyl; and R⁷ is trifluoromethoxy;
   R⁶ is chloro; and R⁷ is trifluoromethyl;
   R⁶ is chloro; and R⁷ is trifluoromethoxy;
   R⁷ is isopropyl; and R⁶ is methyl;
   R⁷ is isopropyl; and R⁶ is trifluoromethyl;
   R⁷ is isopropyl; and R⁶ is cyclopropyl;
   R⁷ is isopropyl; and R⁶ is chloro;
   R⁷ is ethyl, and R⁶ is fluoro;
   R⁷ is isopropyl; and R⁶ is cyano;
   R⁷ is cyclopropyl; and R⁶ is cyclopropyl;
   R⁷ is cyclopropyl; and R⁶ is chloro;
   R⁷ is cyclopropyl; and R⁶ is fluoro;
   R⁷ is isopropyl; and R⁶ is methoxy;
   R⁷ is isopropyl; and R⁶ is trifluoromethoxy;
   R⁷ is chloro; and R⁶ is trifluoromethyl;
   R⁷ is chloro; and R⁶ is trifluoromethoxy;
   R⁶ and R⁷ on adjacent atoms taken together with the atoms connecting them form a C₄ aliphatic carbocyclic ring;
   R⁶ and R⁷ on adjacent atoms taken together with the atoms connecting them form a C₅ aliphatic carbocyclic ring;
   R⁶ and R⁷ on adjacent atoms taken together with the atoms connecting them form a C₆ aliphatic carbocyclic ring;
   R⁶ and R⁷ on adjacent atoms taken together with the atoms connecting them form a 5-membered heterocyclic ring containing 1 heteroatoms independently selected from O, N, and S;
   R⁶ and R⁷ on adjacent atoms taken together with the atoms connecting them form a 6-membered heterocyclic ring containing 1 heteroatoms independently selected from O, N, and S; or
   R⁶ and R⁷ on adjacent atoms taken together with the atoms connecting them form a C₅ aliphatic carbocyclic ring.

In some embodiments, of the compound of formula AA,
the substituted ring B is

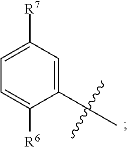

and R⁶ and R⁷ are one of the following combinations:
   R⁶ is C₁-C₆ alkyl, and R⁷ is C₁-C₆ alkyl optionally substituted with one or more halo;
   R⁶ is C₁-C₆ alkyl and R⁷ is C₁-C₆ alkyl;
   R⁶ is C₁-C₆ alkyl, and R⁷ is C₁-C₆ alkyl substituted with one or more halo;
   R⁶ is C₁-C₆ alkyl, and R⁷ is C₃-C₇ cycloalkyl;
   R⁶ is C₁-C₆ alkyl, and R⁷ is halo;
   R⁶ is C₁-C₆ alkyl, and R⁷ is cyano;
   R⁶ is C₃-C₇ cycloalkyl, and R⁷ is C₃-C₇ cycloalkyl;
   R⁶ is C₃-C₇ cycloalkyl, and R⁷ is halo;
   R⁶ is cyclopropyl and R⁷ is halo;
   R⁶ is C₁-C₆ alkyl, and R⁷ is C₁-C₆ alkoxy optionally substituted with one or more halo;
   R⁶ is C₁-C₆ alkyl, and R⁷ is C₁-C₆ alkoxy;
   R⁶ is C₁-C₆ alkyl, and R⁷ is C₁-C₆ alkoxy substituted with one or more halo;
   R⁶ is halo, and R⁷ is C₁-C₆ haloalkyl;
   R⁶ is halo, and R⁷ is C₁-C₆ haloalkoxy;
   R⁶ is C₁-C₆ alkoxy; and R⁷ is halo;
   R⁶ is C₁-C₆ alkoxy; and R⁷ is chloro;
   R⁷ is C₁-C₆ alkyl, and R⁶ is C₁-C₆ alkyl optionally substituted with one or more halo;
   R⁷ is C₁-C₆ alkyl, and R⁶ is C₁-C₆ alkyl substituted with one or more halo;
   R⁷ is C₁-C₆ alkyl, and R⁶ is C₃-C₇ cycloalkyl;
   R⁷ is C₁-C₆ alkyl, and R⁶ is halo;
   R⁷ is C₁-C₆ alkyl and R⁶ is halo;
   R⁷ is C₁-C₆ alkyl, and R⁶ is cyano;
   R⁷ is C₃-C₇ cycloalkyl, and R⁶ is C₃-C₇ cycloalkyl;
   R⁷ is C₃-C₇ cycloalkyl, and R⁶ is halo;
   R⁷ is C₃-C₇ cycloalkyl and R⁶ is halo;
   R⁷ is C₁-C₆ alkyl, and R⁶ is C₁-C₆ alkoxy optionally substituted with one or more halo;
   R⁷ is C₁-C₆ alkyl, and R⁶ is C₁-C₆ alkoxy;
   R⁷ is C₁-C₆ alkyl, and R⁶ is C₁-C₆ alkoxy substituted with one or more halo;
   R⁷ is halo, and R⁶ is C₁-C₆ haloalkyl;
   R⁷ is halo, and R⁶ is C₁-C₆ haloalkoxy;
   R⁷ is C₁-C₆ alkoxy; and R⁶ is halo;
   R⁷ is C₁-C₆ alkoxy; and R⁶ is chloro.

In some embodiments, of the compound of formula AA,
the substituted ring B is

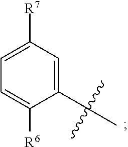

and R⁶ and R⁷ are one of the following combinations:
   R⁶ is isopropyl; and R⁷ is methyl;
   R⁶ is isopropyl; and R⁷ is isopropyl;
   R⁶ is isopropyl; and R⁷ is trifluoromethyl;
   R⁶ is isopropyl; and R⁷ is cyclopropyl;
   R⁶ is isopropyl; and R⁷ is chloro;
   R⁶ is isopropyl; and R⁷ is fluoro;
   R⁶ is ethyl; and R⁷ is fluoro;
   R⁶ is isopropyl; and R⁷ is cyano;
   R⁶ is cyclopropyl; and R⁷ is cyclopropyl;
   R⁶ is cyclopropyl; and R⁷ is chloro;
   R⁶ is cyclopropyl; and R⁷ is fluoro;
   R⁶ is isopropyl; and R⁷ is methoxy;
   R⁶ is isopropyl; and R⁷ is trifluoromethoxy;
   R⁶ is chloro; and R⁷ is trifluoromethyl;
   R⁶ is chloro; and R⁷ is trifluoromethoxy;
   R⁷ is isopropyl; and R⁶ is methyl;
   R⁷ is isopropyl; and R⁶ is trifluoromethyl;
   R⁷ is isopropyl; and R⁶ is cyclopropyl;
   R⁷ is isopropyl; and R⁶ is chloro;
   R⁷ is ethyl; and R⁶ is fluoro;
   R⁷ is isopropyl; and R⁶ is cyano;
   R⁷ is cyclopropyl; and R⁶ is cyclopropyl;
   R⁷ is cyclopropyl; and R⁶ is chloro;
   R⁷ is cyclopropyl; and R⁶ is fluoro;
   R⁷ is isopropyl; and R⁶ is methoxy;
   R⁷ is isopropyl; and R⁶ is trifluoromethoxy;
   R⁷ is chloro; and R⁶ is trifluoromethyl;
   R⁷ is chloro; and R⁶ is trifluoromethoxy.

In some embodiments, of the compound of formula AA,
the substituted ring B is

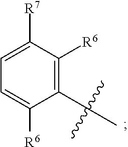

and R⁶ and R⁷ are one of the following combinations:
   each R⁶ is independently C₁-C₆ alkyl, and R⁷ is C₁-C₆ alkyl optionally substituted with one or more halo;
   each R⁶ is independently C₁-C₆ alkyl and R⁷ is C₁-C₆ alkyl;
   each R⁶ is independently C₁-C₆ alkyl, and R⁷ is C₁-C₆ alkyl substituted with one or more halo;
   each R⁶ is independently C₁-C₆ alkyl, and R⁷ is C₃-C₇ cycloalkyl;
   each R⁶ is independently C₁-C₆ alkyl, and R⁷ is halo;
   each R⁶ is independently C₁-C₆ alkyl, and R⁷ is cyano;
   each R⁶ is independently C₃-C₇ cycloalkyl, and R⁷ is C₃-C₇ cycloalkyl;
   each R⁶ is independently C₃-C₇ cycloalkyl, and R⁷ is halo;
   each R⁶ is independently cyclopropyl and R⁷ is halo;
   each R⁶ is independently C₁-C₆ alkyl, and R⁷ is C₁-C₆ alkoxy optionally substituted with one or more halo;
   each R⁶ is independently C₁-C₆ alkyl, and R⁷ is C₁-C₆ alkoxy;
   each R⁶ is independently C₁-C₆ alkyl, and R⁷ is C₁-C₆ alkoxy substituted with one or more halo;
   each R⁶ is independently halo, and R⁷ is C₁-C₆ haloalkyl;
   each R⁶ is independently halo, and R⁷ is C₁-C₆ haloalkoxy;
   each R⁶ is independently C₁-C₆ alkoxy; and R⁷ is halo;
   each R⁶ is independently C₁-C₆ alkoxy; and R⁷ is chloro;
   R⁷ is C₁-C₆ alkyl, and each R⁶ is independently C₁-C₆ alkyl optionally substituted with one or more halo;
   R⁷ is C₁-C₆ alkyl, and each R⁶ is independently C₁-C₆ alkyl substituted with one or more halo;
   R⁷ is C₁-C₆ alkyl, and each R⁶ is independently C₃-C₇ cycloalkyl;
   R⁷ is C₁-C₆ alkyl, and each R⁶ is independently halo;
   R⁷ is C₁-C₆ alkyl and each R⁶ is independently halo;
   R⁷ is C₁-C₆ alkyl, and each R⁶ is cyano;
   R⁷ is C₃-C₇ cycloalkyl, and each R⁶ is independently C₃-C₇ cycloalkyl;
   R⁷ is C₃-C₇ cycloalkyl, and each R⁶ is independently halo;
   R⁷ is C₃-C₇ cycloalkyl and each R⁶ is independently halo;
   R⁷ is C₁-C₆ alkyl, and each R⁶ is independently C₁-C₆ alkoxy optionally substituted with one or more halo;
   R⁷ is C₁-C₆ alkyl, and each R⁶ is independently C₁-C₆ alkoxy;
   R⁷ is C₁-C₆ alkyl, and each R⁶ is independently C₁-C₆ alkoxy substituted with one or more halo;
   R⁷ is halo, and each R⁶ is independently C₁-C₆ haloalkyl;
   R⁷ is halo, and each R⁶ is independently C₁-C₆ haloalkoxy;
   R⁷ is C₁-C₆ alkoxy; and each R⁶ is independently halo;
   R⁷ is C₁-C₆ alkoxy; and each R⁶ is chloro;
   R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,
the substituted ring B is

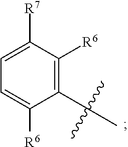

and R⁶ and R⁷ are one of the following combinations:
   each R⁶ is isopropyl; and R⁷ is methyl;
   each R⁶ is isopropyl; and R⁷ is isopropyl;
   each R⁶ is isopropyl; and R⁷ is trifluoromethyl;
   each R⁶ is isopropyl; and R⁷ is cyclopropyl;
   each R⁶ is isopropyl; and R⁷ is chloro;
   each R⁶ is isopropyl; and R⁷ is fluoro;
   each R⁶ is ethyl; and R⁷ is fluoro;
   each R⁶ is isopropyl; and R⁷ is cyano;
   each R⁶ is cyclopropyl; and R⁷ is cyclopropyl;
   each R⁶ is cyclopropyl; and R⁷ is chloro;
   each R⁶ is cyclopropyl; and R⁷ is fluoro;
   each R⁶ is isopropyl; and R⁷ is methoxy;
   each R⁶ is isopropyl; and R⁷ is trifluoromethoxy;
   each R⁶ is chloro; and R⁷ is trifluoromethyl;
   each R⁶ is chloro; and R⁷ is trifluoromethoxy;
   R⁷ is isopropyl; and each R⁶ is methyl;
   R⁷ is isopropyl; and each R⁶ is trifluoromethyl;
   R⁷ is isopropyl; and each R⁶ is cyclopropyl;
   R⁷ is isopropyl; and each R⁶ is chloro;
   R⁷ is ethyl; and each R⁶ is fluoro;
   R⁷ is isopropyl; and each R⁶ is cyano;
   R⁷ is cyclopropyl; and each R⁶ is cyclopropyl;
   R⁷ is cyclopropyl; and each R⁶ is chloro;
   R⁷ is cyclopropyl; and each R⁶ is fluoro;
   R⁷ is isopropyl; and each R⁶ is methoxy;
   R⁷ is isopropyl; and each R⁶ is trifluoromethoxy;
   R⁷ is chloro; and each R⁶ is trifluoromethyl;
   R⁷ is chloro; and each R⁶ is trifluoromethoxy;
   one R⁶ is isopropyl; the other R⁶ is trifluoromethyl; and R⁷ is chloro;
   R⁶ and R⁷ on adjacent atoms taken together with the atoms connecting them form a C₄ aliphatic carbocyclic ring; and one R⁶ is chloro, fluoro, or cyano;
   R⁶ and R⁷ on adjacent atoms taken together with the atoms connecting them form a C₅ aliphatic carbocyclic ring; and one R⁶ is chloro, fluoro, or cyano;
   R⁶ and R⁷ on adjacent atoms taken together with the atoms connecting them form a C₆ aliphatic carbocyclic ring; and one R⁶ is chloro, fluoro, or cyano;
   R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
   R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
   R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,
the substituted ring B is

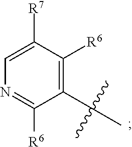

and R⁶ and R⁷ are one of the following combinations:
   each R⁶ is independently C₁-C₆ alkyl, and R⁷ is C₁-C₆ alkyl optionally substituted with one or more halo;
   each R⁶ is independently C₁-C₆ alkyl and R⁷ is C₁-C₆ alkyl;
   each R⁶ is independently C₁-C₆ alkyl, and R⁷ is C₁-C₆ alkyl substituted with one or more halo;
   each R⁶ is independently C₁-C₆ alkyl, and R⁷ is C₃-C₇ cycloalkyl;
   each R⁶ is independently C₁-C₆ alkyl, and R⁷ is halo;
   each R⁶ is independently C₁-C₆ alkyl, and R⁷ is cyano;
   each R⁶ is independently C₃-C₇ cycloalkyl, and R⁷ is C₃-C₇ cycloalkyl;
   each R⁶ is independently C₃-C₇ cycloalkyl, and R⁷ is halo;
   each R⁶ is independently cyclopropyl and R⁷ is halo;
   each R⁶ is independently C₁-C₆ alkyl, and R⁷ is C₁-C₆ alkoxy optionally substituted with one or more halo;
   each R⁶ is independently C₁-C₆ alkyl, and R⁷ is C₁-C₆ alkoxy;
   each R⁶ is independently C₁-C₆ alkyl, and R⁷ is C₁-C₆ alkoxy substituted with one or more halo;
   each R⁶ is independently halo, and R⁷ is C₁-C₆ haloalkyl;
   each R⁶ is independently halo, and R⁷ is C₁-C₆ haloalkoxy;
   each R⁶ is independently C₁-C₆ alkoxy; and R⁷ is halo;
   each R⁶ is independently C₁-C₆ alkoxy; and R⁷ is chloro;
   R⁷ is C₁-C₆ alkyl, and each R⁶ is independently C₁-C₆ alkyl optionally substituted with one or more halo;
   R⁷ is C₁-C₆ alkyl, and each R⁶ is independently C₁-C₆ alkyl substituted with one or more halo;
   R⁷ is C₁-C₆ alkyl, and each R⁶ is independently C₃-C₇ cycloalkyl;
   R⁷ is C₁-C₆ alkyl, and each R⁶ is independently halo;
   R⁷ is C₁-C₆ alkyl and each R⁶ is independently halo;
   R⁷ is C₁-C₆ alkyl, and each R⁶ is cyano;
   R⁷ is C₃-C₇ cycloalkyl, and each R⁶ is independently C₃-C₇ cycloalkyl;
   R⁷ is C₃-C₇ cycloalkyl, and each R⁶ is independently halo;
   R⁷ is C₃-C₇ cycloalkyl and each R⁶ is independently halo;
   R⁷ is C₁-C₆ alkyl, and each R⁶ is independently C₁-C₆ alkoxy optionally substituted with one or more halo;
   R⁷ is C₁-C₆ alkyl, and each R⁶ is independently C₁-C₆ alkoxy;
   R⁷ is C₁-C₆ alkyl, and each R⁶ is independently C₁-C₆ alkoxy substituted with one or more halo;
   R⁷ is halo, and each R⁶ is independently C₁-C₆ haloalkyl;
   R⁷ is halo, and each R⁶ is independently C₁-C₆ haloalkoxy;
   R⁷ is C₁-C₆ alkoxy; and each R⁶ is independently halo;
   R⁷ is C₁-C₆ alkoxy; and each R⁶ is chloro;
   R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,
the substituted ring B is

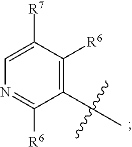

and R⁶ and R⁷ are one of the following combinations:
   each R⁶ is isopropyl; and R⁷ is methyl;
   each R⁶ is isopropyl; and R⁷ is isopropyl;
   each R⁶ is isopropyl; and R⁷ is trifluoromethyl;
   each R⁶ is isopropyl; and R⁷ is cyclopropyl;
   each R⁶ is isopropyl; and R⁷ is chloro;
   each R⁶ is isopropyl; and R⁷ is fluoro;
   each R⁶ is ethyl; and R⁷ is fluoro;
   each R⁶ is isopropyl; and R⁷ is cyano;
   each R⁶ is cyclopropyl; and R⁷ is cyclopropyl;
   each R⁶ is cyclopropyl; and R⁷ is chloro;
   each R⁶ is cyclopropyl; and R⁷ is fluoro;
   each R⁶ is isopropyl; and R⁷ is methoxy;
   each R⁶ is isopropyl; and R⁷ is trifluoromethoxy;
   each R⁶ is chloro; and R⁷ is trifluoromethyl;
   each R⁶ is chloro; and R⁷ is trifluoromethoxy;
   R⁷ is isopropyl; and each R⁶ is methyl;
   R⁷ is isopropyl; and each R⁶ is trifluoromethyl;
   R⁷ is isopropyl; and each R⁶ is cyclopropyl;
   R⁷ is isopropyl; and each R⁶ is chloro;
   R⁷ is ethyl; and each R⁶ is fluoro;
   R⁷ is isopropyl; and each R⁶ is cyano;
   R⁷ is cyclopropyl; and each R⁶ is cyclopropyl;
   R⁷ is cyclopropyl; and each R⁶ is chloro;
   R⁷ is cyclopropyl; and each R⁶ is fluoro;
   R⁷ is isopropyl; and each R⁶ is methoxy;
   R⁷ is isopropyl; and each R⁶ is trifluoromethoxy;
   R⁷ is chloro; and each R⁶ is trifluoromethyl;
   R⁷ is chloro; and each R⁶ is trifluoromethoxy;
   one R⁶ is isopropyl; the other R⁶ is trifluoromethyl; and R⁷ is chloro;
   R⁶ and R⁷ on adjacent atoms taken together with the atoms connecting them form a C₄ aliphatic carbocyclic ring; and one R⁶ is chloro, fluoro, or cyano;
   R⁶ and R⁷ on adjacent atoms taken together with the atoms connecting them form a C₅ aliphatic carbocyclic ring; and one R⁶ is chloro, fluoro, or cyano;
   R⁶ and R⁷ on adjacent atoms taken together with the atoms connecting them form a C₆ aliphatic carbocyclic ring; and one R⁶ is chloro, fluoro, or cyano;
   R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
   R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,
the substituted ring B is

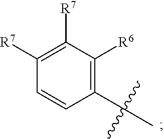

and R⁶ and R⁷ are one of the following combinations:
   R⁶ is C₁-C₆ alkyl, and each R⁷ is independently C₁-C₆ alkyl optionally substituted with one or more halo;
   R⁶ is C₁-C₆ alkyl and each R⁷ is independently C₁-C₆ alkyl;
   R⁶ is C₁-C₆ alkyl, and each R⁷ is independently C₁-C₆ alkyl substituted with one or more halo;
   R⁶ is C₁-C₆ alkyl, and each R⁷ is independently C₃-C₇ cycloalkyl;
   R⁶ is C₁-C₆ alkyl, and each R⁷ is independently halo;
   R⁶ is C₁-C₆ alkyl, and each R⁷ is cyano;
   R⁶ is C₃-C₇ cycloalkyl, and each R⁷ is independently C₃-C₇ cycloalkyl;
   R⁶ is C₃-C₇ cycloalkyl, and each R⁷ is independently halo,
   R⁶ is cyclopropyl and each R⁷ is independently halo;
   R⁶ is C₁-C₆ alkyl, and each R⁷ is independently C₁-C₆ alkoxy optionally substituted with one or more halo;
   R⁶ is C₁-C₆ alkyl, and each R⁷ is independently C₁-C₆ alkoxy;
   R⁶ is C₁-C₆ alkyl, and each R⁷ is independently C₁-C₆ alkoxy substituted with one or more halo;
   R⁶ is halo, and each R⁷ is independently C₁-C₆ haloalkyl;
   R⁶ is halo, and each R⁷ is independently C₁-C₆ haloalkoxy;
   R⁶ is C₁-C₆ alkoxy; and each R⁷ is independently halo;
   R⁶ is C₁-C₆ alkoxy; and each R⁷ is chloro;
   each R⁷ is independently C₁-C₆ alkyl, and R⁶ is C₁-C₆ alkyl optionally substituted with one or more halo;
   each R⁷ is independently C₁-C₆ alkyl, and R⁶ is C₁-C₆ alkyl substituted with one or more halo;
   each R⁷ is independently C₁-C₆ alkyl, and R⁶ is C₃-C₇ cycloalkyl;
   each R⁷ is independently C₁-C₆ alkyl, and R⁶ is halo;
   each R⁷ is independently C₁-C₆ alkyl and R⁶ is halo;
   each R⁷ is independently C₁-C₆ alkyl, and R⁶ is cyano;
   each R⁷ is independently C₃-C₇ cycloalkyl, and R⁶ is C₃-C₇ cycloalkyl;
   each R⁷ is independently C₃-C₇ cycloalkyl, and R⁶ is halo;
   each R⁷ is independently C₃-C₇ cycloalkyl and R⁶ is halo;
   each R⁷ is independently C₁-C₆ alkyl, and R⁶ is C₁-C₆ alkoxy optionally substituted with one or more halo;
   each R⁷ is independently C₁-C₆ alkyl, and R⁶ is C₁-C₆ alkoxy;
   each R⁷ is independently C₁-C₆ alkyl, and R⁶ is C₁-C₆ alkoxy substituted with one or more halo;
   each R⁷ is independently halo, and R⁶ is C₁-C₆ haloalkyl;
   each R⁷ is independently halo, and R⁶ is C₁-C₆ haloalkoxy;
   each R⁷ is independently C₁-C₆ alkoxy; and R⁶ is halo;
   each R⁷ is independently C₁-C₆ alkoxy; and R⁶ is chloro.

In some embodiments, of the compound of formula AA,
the substituted ring B is

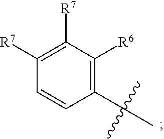

and R⁶ and R⁷ are one of the following combinations:
   each R⁷ is isopropyl; and R⁶ is methyl;
   each R⁷ is isopropyl; and R⁶ is isopropyl;
   each R⁷ is isopropyl; and R⁶ is trifluoromethyl;
   each R⁷ is isopropyl; and R⁶ is cyclopropyl;
   each R⁷ is isopropyl; and R⁶ is chloro;
   each R⁷ is isopropyl; and R⁶ is fluoro;
   each R⁷ is ethyl; and R⁶ is fluoro;
   each R⁷ is isopropyl; and R⁶ is cyano;
   each R⁷ is cyclopropyl; and R⁶ is cyclopropyl;
   each R⁷ is cyclopropyl; and R⁶ is chloro;
   each R⁷ is cyclopropyl; and R⁶ is fluoro;
   each R⁷ is isopropyl; and R⁶ is methoxy;
   each R⁷ is isopropyl; and R⁶ is trifluoromethoxy;
   each R⁷ is chloro; and R⁶ is trifluoromethyl;
   each R⁷ is chloro; and R⁶ is trifluoromethoxy;
   R⁷ is isopropyl; and each R⁶ is methyl;
   R⁷ is isopropyl; and each R⁶ is trifluoromethyl;
   R⁷ is isopropyl; and each R⁶ is cyclopropyl;
   R⁷ is isopropyl; and each R⁶ is chloro;
   R⁷ is ethyl; and each R⁶ is fluoro;
   R⁷ is isopropyl; and each R⁶ is cyano;
   R⁷ is cyclopropyl; and each R⁶ is cyclopropyl;
   R⁷ is cyclopropyl; and each R⁶ is chloro;
   R⁷ is cyclopropyl; and each R⁶ is fluoro;
   R⁷ is isopropyl; and each R⁶ is methoxy;
   R⁷ is isopropyl; and each R⁶ is trifluoromethoxy;
   R⁷ is chloro; and each R⁶ is trifluoromethyl;
   R⁷ is chloro; and each R⁶ is trifluoromethoxy.

In some embodiments, of the compound of formula AA,
the substituted ring B is

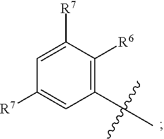

and R⁶ and Rare one of the following combinations;
   R⁶ is C₁-C₆ alkyl, and each R⁷ is independently C₁-C₆ alkyl optionally substituted with one or more halo;
   R⁶ is C₁-C₆ alkyl and each R⁷ is independently C₁-C₆ alkyl;
   R⁶ is C₁-C₆ alkyl, and each R⁷ is independently C₁-C₆ alkyl substituted with one or more halo;
   R⁶ is C₁-C₆ alkyl, and each R⁷ is independently C₃-C₇ cycloalkyl;
   R⁶ is C₁-C₆ alkyl, and each R⁷ is independently halo;
   R⁶ is C₁-C₆ alkyl, and each R⁷ is cyano;
   R⁶ is C₃-C₇ cycloalkyl, and each R⁷ is independently C₃-C₇ cycloalkyl;
   R⁶ is C₃-C₇ cycloalkyl, and each R⁷ is independently halo;
   R⁶ is cyclopropyl and each R⁷ is independently halo;
   R⁶ is C₁-C₆ alkyl, and each R⁷ is independently C₁-C₆ alkoxy optionally substituted with one or more halo;
   R⁶ is C₁-C₆ alkyl, and each R⁷ is independently C₁-C₆ alkoxy;
   R⁶ is C₁-C₆ alkyl, and each R⁷ is independently C₁-C₆ alkoxy substituted with one or more halo;
   R⁶ is halo, and each R⁷ is independently C₁-C₆ haloalkyl;
   R⁶ is halo, and each R⁷ is independently C₁-C₆ haloalkoxy;
   R⁶ is C₁-C₆ alkoxy; and each R⁷ is independently halo;
   R⁶ is C₁-C₆ alkoxy; and each R⁷ is chloro;
   each R⁷ is independently C₁-C₆ alkyl, and R⁶ is C₁-C₆ alkyl optionally substituted with one or more halo;
   each R⁷ is independently C₁-C₆ alkyl, and R⁶ is C₁-C₆ alkyl substituted with one or more halo;
   each R⁷ is independently C₁-C₆ alkyl, and R⁶ is C₃-C₇ cycloalkyl;
   each R⁷ is independently C₁-C₆ alkyl, and R⁶ is halo;
   each R⁷ is independently C₁-C₆ alkyl and R⁶ is halo;
   each R⁷ is independently C₁-C₆ alkyl, and R⁶ is cyano;
   each R⁷ is independently C₃-C₇ cycloalkyl, and R⁶ is C₃-C₇ cycloalkyl;
   each R⁷ is independently C₃-C₇ cycloalkyl, and R⁶ is halo;
   each R⁷ is independently C₃-C₇ cycloalkyl and R⁶ is halo;
   each R⁷ is independently C₁-C₆ alkyl, and R⁶ is C₁-C₆ alkoxy optionally substituted with one or more halo;
   each R⁷ is independently C₁-C₆ alkyl, and R⁶ is C₁-C₆ alkoxy;
   each R⁷ is independently C₁-C₆ alkyl, and R⁶ is C₁-C₆ alkoxy substituted with one or more halo;
   each R⁷ is independently halo, and R⁶ is C₁-C₆ haloalkyl;
   each R⁷ is independently halo, and R⁶ is C₁-C₆ haloalkoxy;
   each R⁷ is independently C₁-C₆ alkoxy; and R⁶ is halo;
   each R⁶ is independently C₁-C₆ alkoxy; and R⁶ is chloro.

In some embodiments, of the compound of formula AA,
the substituted ring B is

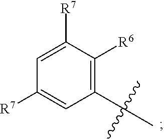

and R⁶ and R⁷ are one of the following combinations:
   R⁶ is isopropyl; and each R⁷ is methyl;
   R⁶ is isopropyl; and each R⁷ is isopropyl;
   R⁶ is isopropyl; and each R⁷ is trifluoromethyl;
   R⁶ is isopropyl; and each R⁷ is cyclopropyl;
   R⁶ is isopropyl; and each R⁷ is chloro;
   R⁶ is isopropyl; and each R⁷ is fluoro;
   R⁶ is ethyl; and each R⁷ is fluoro;
   R⁶ is isopropyl; and each R⁷ is cyano;
   R⁶ is cyclopropyl; and each R⁷ is cyclopropyl;
   R⁶ is cyclopropyl; and each R⁷ is chloro;
   R⁶ is cyclopropyl; and each R⁷ is fluoro;
   R⁶ is isopropyl; and each R⁷ is methoxy;
   R⁶ is isopropyl; and R⁷ is trifluoromethoxy;
   R⁶ is chloro; and each R⁷ is trifluoromethyl;
   R⁶ is chloro; and each R⁷ is trifluoromethoxy;
   each R⁷ is isopropyl; and R⁶ is methyl;
   each R⁷ is isopropyl; and R⁶ is trifluoromethyl;
   each R⁷ is isopropyl; and R⁶ is cyclopropyl;
   each R⁷ is isopropyl; and R⁶ is chloro;
   each R⁷ is ethyl; and R⁶ is fluoro;
   each R⁷ is isopropyl; and R⁶ is cyano;
   each R⁷ is cyclopropyl; and R⁶ is cyclopropyl;
   each R⁷ is cyclopropyl; and R⁶ is chloro;
   each R⁷ is cyclopropyl; and R⁶ is fluoro;
   each R⁷ is isopropyl; and R⁶ is methoxy;
   each R⁷ is isopropyl; and R⁶ is trifluoromethoxy;
   each R⁷ is chloro; and R⁶ is trifluoromethyl;
   each R⁷ is chloro; and R⁶ is trifluoromethoxy.

In some embodiments, of the compound of formula AA,
the substituted ring B is

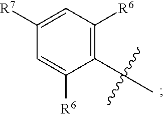

and R⁶ and R⁷ are one of the following combinations:
   each R⁶ is independently C₁-C₆ alkyl, and R⁷ is C₁-C₆ alkyl optionally substituted with one or more halo;
   each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo;
   each R⁶ is independently C₁-C₆ alkyl, and R⁷ is C₃-C₇ cycloalkyl;
   each R⁶ is independently C₁-C₆ alkyl, and R⁷ is halo;
   each R⁶ is independently C₁-C₆ alkyl, and R⁷ is cyano;
   each R⁶ is independently C₃-C₇ cycloalkyl, and R⁷ is C₃-C₇ cycloalkyl;
   each R⁶ is independently C₃-C₇ cycloalkyl, and R⁷ is halo;
   each R⁶ is independently cyclopropyl and R⁷ is halo;
   each R⁶ is independently C₁-C₆ alkyl, and R⁷ is C₁-C₆ alkoxy optionally substituted with one or more halo;
   each R⁶ is independently C₁-C₆ alkyl, and R⁷ is C₁-C₆ alkoxy;
   each R⁶ is independently C₁-C₆ alkyl, and R⁷ is C₁-C₆ alkoxy substituted with one or more halo;
   each R⁶ is independently halo, and R⁷ is C₁-C₆ haloalkyl;
   each R⁶ is independently halo, and R⁷ is C₁-C₆ haloalkoxy;
   each R⁶ is independently C₁-C₆ alkoxy; and R⁷ is halo;
   each R⁶ is independently C₁-C₆ alkoxy; and R⁷ is chloro;
   R⁷ is C₁-C₆ alkyl, and each R⁶ is independently C₁-C₆ alkyl optionally substituted with one or more halo;
   R⁷ is C₁-C₆ alkyl, and each R⁶ is independently C₁-C₆ alkyl substituted with one or more halo;
   R⁷ is C₁-C₆ alkyl, and each R⁶ is independently C₃-C₇ cycloalkyl;
   R⁷ is C₁-C₆ alkyl, and each R⁶ is independently halo;
   R⁷ is C₁-C₆ alkyl and each R⁶ is independently halo;
   R⁷ is C₁-C₆ alkyl, and each R⁶ is cyano;
   R⁷ is C₁-C₇ cycloalkyl, and each R⁶ is independently C₃-C₇ cycloalkyl;
   R⁷ is C₃-C₇ cycloalkyl, and each R⁶ is independently halo;
   R⁷ is C₃-C₇ cycloalkyl and each R⁶ is independently halo;
   R⁷ is C₁-C₆ alkyl, and each R⁶ is independently C₁-C₆ alkoxy optionally substituted with one or more halo;
   R⁷ is C₁-C₆ alkyl, and each R⁶ is independently C₁-C₆ alkoxy;
   R⁷ is C₁-C₆ alkyl, and each R⁶ is independently C₁-C₆ alkoxy substituted with one or more halo;
   R⁷ is halo, and each R⁶ is independently C₁-C₆ haloalkyl;
   R⁷ is halo, and each R⁶ is independently C₁-C₆ haloalkoxy;
   R⁷ is C₁-C₆ alkoxy; and each R⁶ is independently halo;
   R⁷ is C₁-C₆ alkoxy; and each R⁶ is chloro.

In some embodiments, of the compound of formula AA,
the substituted ring B is

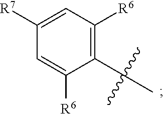

and R⁶ and R⁷ are one of the following combinations:
   each R⁶ is isopropyl; and R⁷ is methyl;
   each R⁶ is isopropyl; and R⁷ is isopropyl;
   each R⁶ is isopropyl; and R⁷ is trifluoromethyl;
   each R⁶ is isopropyl; and R⁷ is cyclopropyl;
   each R⁶ is isopropyl; and R⁷ is chloro;
   each R⁶ is isopropyl; and R⁷ is fluoro;
   each R⁶ is ethyl; and R⁷ is fluoro;
   each R⁶ is isopropyl; and R⁷ is cyano;
   each R⁶ is cyclopropyl; and R⁷ is cyclopropyl;
   each R⁶ is cyclopropyl; and R⁷ is chloro;
   each R⁶ is cyclopropyl; and R⁷ is fluoro;
   each R⁶ is isopropyl; and R⁷ is methoxy;
   each R⁶ is isopropyl; and R⁷ is trifluoromethoxy;
   each R⁶ is chloro; and R⁷ is trifluoromethyl;
   each R⁶ is chloro; and R⁷ is trifluoromethoxy;
   R⁷ is isopropyl; and each R⁶ is methyl;
   R⁷ is isopropyl; and each R⁶ is trifluoromethyl;
   R⁷ is isopropyl; and each R⁶ is cyclopropyl;
   R⁷ is isopropyl; and each R⁶ is chloro;
   R⁷ is ethyl; and each R⁶ is fluoro;
   R⁷ is isopropyl; and each R⁶ is cyano;
   R⁷ is cyclopropyl; and each R⁶ is cyclopropyl;
   R⁷ is cyclopropyl; and each R⁶ is chloro;
   R⁷ is cyclopropyl; and each R⁶ is fluoro;
   R⁷ is isopropyl; and each R⁶ is methoxy;
   R⁷ is isopropyl; and each R⁶ is trifluoromethoxy;
   R⁷ is chloro; and each R⁶ is trifluoromethyl;
   R⁷ is chloro; and each R⁶ is trifluoromethoxy;
   one R⁶ is isopropyl; the other R⁶ is trifluoromethyl; and R⁷ is chloro.

In some embodiments, of the compound of formula AA,
the substituted ring B is

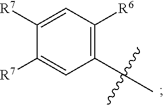

and R⁶ and R⁷ are one of the following combinations:
   R⁶ is C₁-C₆ alkyl, and each R⁷ is independently C₁-C₆ alkyl optionally substituted with one or more halo;
   R⁶ is C₁-C₆ alkyl and each R⁷ is independently C₁-C₆ alkyl;
   R⁶ is C₁-C₆ alkyl, and each R⁷ is independently C₁-C₆ alkyl substituted with one or more halo;
   R⁶ is C₁-C₆ alkyl, and each R⁷ is independently C₃-C₇ cycloalkyl;
   R⁶ is C₁-C₆ alkyl, and each R⁷ is independently halo;
   R⁶ is C₁-C₆ alkyl, and each R⁷ is cyano;
   R⁶ is C₃-C₇ cycloalkyl, and each R⁷ is independently C₁-C₇ cycloalkyl;
   R⁶ is C₃-C₇ cycloalkyl, and each R⁷ is independently halo;
   R⁶ is cyclopropyl and each R⁷ is independently halo;
   R⁶ is C₁-C₆ alkyl, and each R⁷ is independently C₁-C₆ alkoxy optionally substituted with one or more halo;
   R⁶ is C₁-C₆ alkyl, and each R⁷ is independently C₁-C₆ alkoxy;
   R⁶ is C₁-C₆ alkyl, and each R⁷ is independently C₁-C₆ alkoxy substituted with one or more halo;
   R⁶ is halo, and each R⁷ is independently C₁-C₆ haloalkyl;
   R⁶ is halo, and each R⁷ is independently C₁-C₆ haloalkoxy;
   R⁶ is C₁-C₆ alkoxy; and each R⁷ is independently halo;
   R⁶ is C₁-C₆ alkoxy; and each R⁷ is chloro;
   each R⁷ is independently C₁-C₆ alkyl, and R⁶ is C₁-C₆ alkyl optionally substituted with one or more halo;
   each R⁷ is independently C₁-C₆ alkyl, and R⁶ is C₁-C₆ alkyl substituted with one or more halo;
   each R⁷ is independently C₁-C₆ alkyl, and R⁶ is C₃-C₇ cycloalkyl;
   each R⁷ is independently C₁-C₆ alkyl, and R⁶ is halo;
   each R⁷ is independently C₁-C₆ alkyl and R⁶ is halo;
   each R⁷ is independently C₁-C₆ alkyl, and R⁶ is cyano;
   each R⁷ is independently C₃-C₇ cycloalkyl, and R⁶ is C₃-C₇ cycloalkyl;
   each R⁷ is independently C₃-C₇ cycloalkyl, and R⁶ is halo;
   each R⁷ is independently C₃-C₇ cycloalkyl and R⁶ is halo;
   each R⁷ is independently C₁-C₆ alkyl, and R⁶ is C₁-C₆ alkoxy optionally substituted with one or more halo;
   each R⁷ is independently C₁-C₆ alkyl, and R⁶ is C₁-C₆ alkoxy;
   each R⁷ is independently C₁-C₆ alkyl, and R⁶ is C₁-C₆ alkoxy substituted with one or more halo;
   each R⁷ is independently halo, and R⁶ is C₁-C₆ haloalkyl;
   each R⁷ is independently halo, and R⁶ is C₁-C₆ haloalkoxy;
   each R⁷ is independently C₁-C₆ alkoxy; and R⁶ is halo;
   each R⁷ is independently C₁-C₆ alkoxy; and R⁶ is chloro.

In some embodiments, of the compound of formula AA,
the substituted ring B is

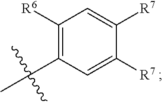

and R⁶ and R⁷ are one of the following combinations:
   R⁶ is isopropyl; and each R⁷ is methyl;
   R⁶ is isopropyl; and each R⁷ is isopropyl;
   R⁶ is isopropyl; and each R⁷ is trifluoromethyl;
   R⁶ is isopropyl; and each R⁷ is cyclopropyl;
   R⁶ is isopropyl; and each R⁷ is chloro;
   R⁶ is isopropyl; and each R⁷ is fluoro;
   R⁶ is ethyl; and each R⁷ is fluoro;
   R⁶ is isopropyl; and each R⁷ is cyano;
   R⁶ is cyclopropyl; and each R⁷ is cyclopropyl;
   R⁶ is cyclopropyl; and each R⁷ is chloro;
   R⁶ is cyclopropyl; and each R⁷ is fluoro;
   R⁶ is isopropyl; and each R⁷ is methoxy;
   R⁶ is isopropyl; and each R⁷ is trifluoromethoxy;
   R⁶ is chloro; and each R⁷ is trifluoromethyl;
   R⁶ is chloro; and each R⁷ is trifluoromethoxy;
   each R⁷ is isopropyl; and R⁶ is methyl;
   each R⁷ is isopropyl; and R⁶ is trifluoromethyl;
   each R⁷ is isopropyl; and R⁶ is cyclopropyl;
   each R⁷ is isopropyl; and R⁶ is chloro;
   each R⁷ is ethyl; and R⁶ is fluoro;
   each R⁷ is isopropyl; and R⁶ is cyano;
   each R⁷ is cyclopropyl; and R⁶ is cyclopropyl;
   each R⁷ is cyclopropyl; and R⁶ is chloro;
   each R⁷ is cyclopropyl; and R⁶ is fluoro;
   each R⁷ is isopropyl; and R⁶ is methoxy;
   each R⁷ is isopropyl; and R⁶ is trifluoromethoxy;
   each R⁷ is chloro; and R⁶ is trifluoromethyl;
   each R⁷ is chloro; and R⁶ is trifluoromethoxy.

In some embodiments, of the compound of formula AA,
the substituted ring B is

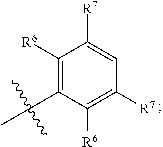

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl;
   each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and each R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and each R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and each R⁶ is chloro;
   two pairs, each of one R⁶ and one R⁷, are on adjacent atoms, and each pair of one R⁶ and one
   R⁷ taken together with the atoms connecting them form a C₄-C₈ aliphatic carbocyclic ring; two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl; or
   two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,
the substituted ring B is

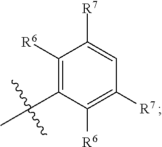

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano;
   two pairs, each of one R⁶ and one R⁷, are on adjacent atoms, and each pair of one R⁶ and one R¹ taken together with the atoms connecting them form a C₈ aliphatic carbocyclic ring; two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl;
   two pairs, each of one R⁶ and one R⁷, are on adjacent atoms, and each pair of one R⁶ and one R¹ taken together with the atoms connecting them form a C₈ aliphatic carbocyclic ring optionally substituted with one or more hydroxy, oxo, or methyl;
   two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl;
   two pairs, each of one R⁶ and one R⁷, are on adjacent atoms, and each pair of one R⁶ and one R¹ taken together with the atoms connecting them form a 5-membered heterocyclic ring containing 1 heteroatom independently selected from O, N, and S, wherein the heterocyclic ring is optionally substituted with one or more hydroxy, oxo, or methyl; or
   two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl.

In some embodiments, of the compound of formula AA,
the substituted ring B is

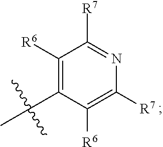

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl;
   each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and each R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and each R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and each R⁶ is chloro;
   two pairs, each of one R⁶ and one R⁷, are on adjacent atoms, and each pair of one R⁶ and one R⁷ taken together with the atoms connecting them form a C₄-C₈ aliphatic carbocyclic ring;
   two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl; or
   two pairs, each of one R⁶ and one R⁷, are on adjacent atoms, and each pair of one R⁶ and one R¹ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,
the substituted ring B is

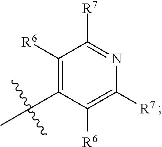

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano;
   two pairs, each of one R⁶ and one R⁷, are on adjacent atoms, and each pair of one R⁶ and one R⁷ taken together with the atoms connecting them form a C₈ aliphatic carbocyclic ring;
   two pairs, each of one R⁶ and one R⁷, are on adjacent atoms, and each pair of one R⁶ and one R⁷ taken together with the atoms connecting them form a Cd aliphatic carbocyclic ring optionally substituted with one or more hydroxy, oxo, or methyl;
   two pairs, each of one R⁶ and one R⁷, are on adjacent atoms, and each pair of one R⁶ and one R⁷ taken together with the atoms connecting them form a C₈ aliphatic carbocyclic ring optionally substituted with one or more hydroxy, oxo, or methyl;
   two pairs, each of one R⁶ and one R⁷, are on adjacent atoms, and each pair of one R⁶ and one R¹ taken together with the atoms connecting them form a C₆ aliphatic carbocyclic ring optionally substituted with one or more hydroxy, oxo, or methyl;
   two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; or
   two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl.

In some embodiments, of the compound of formula AA,
the substituted ring B is

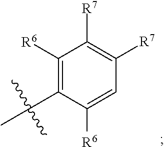

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl: each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and each R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and each R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and each R⁶ is chloro.

In some embodiments, of the compound of formula AA,
the substituted ring B is

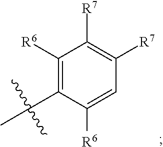

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano.

In some embodiments, of the compound of formula AA,
the substituted ring B is

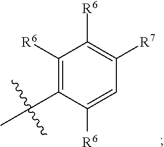

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl;
   each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and each R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and each R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and each R⁶ is chloro;
   R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,
the substituted ring B is

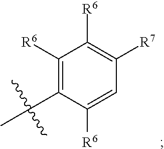

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano;
   R⁶ and R⁷ on adjacent atoms taken together with the atoms connecting them form a C₄ aliphatic carbocyclic ring; and one R⁶ is chloro, fluoro, or cyano;
   R⁶ and R⁷ on adjacent atoms taken together with the atoms connecting them form a C₅ aliphatic carbocyclic ring; and one R⁶ is chloro, fluoro, or cyano;
   R⁶ and R⁷ on adjacent atoms taken together with the atoms connecting them form a C₆ aliphatic carbocyclic ring; and one R⁶ is chloro, fluoro, or cyano;
   R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
   R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
   R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,
the substituted ring B is

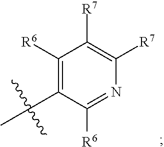

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl;
   each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and R⁶ is chloro.

In some embodiments, of the compound of formula AA,
the substituted ring B is

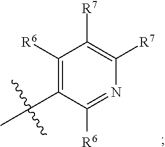

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano.

In some embodiments, of the compound of formula AA,
the substituted ring B is

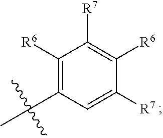

and R⁶ and R⁷ are one of the following combinations:
   each R⁶ is independently C₁-C₆ alkyl, and each R⁷ is independently C₁-C₆ alkyl optionally substituted with one or more halo;
   each R⁶ is independently C₁-C₆ alkyl and each R⁷ is independently C₁-C₆ alkyl;
   each R⁶ is independently C₁-C₆ alkyl, and each R⁷ is independently C₁-C₆ alkyl substituted with one or more halo;
   each R⁶ is independently C₁-C₆ alkyl, and each R⁷ is independently C₃-C₇ cycloalkyl;
   each R⁶ is independently C₁-C₆ alkyl, and each R⁷ is independently halo;
   each R⁶ is independently C₁-C₆ alkyl, and each R⁷ is cyano;
   each R⁶ is independently C₃-C₇ cycloalkyl, and each R⁷ is independently C₃-C₇ cycloalkyl;
   each R⁶ is independently C₃-C₇ cycloalkyl, and each R⁷ is independently halo;
   each R⁶ is independently cyclopropyl and each R⁷ is independently halo;
   each R⁶ is independently C₁-C₆ alkyl, and each R⁷ is independently C₁-C₆ alkoxy optionally substituted with one or more halo;
   each R⁶ is independently C₁-C₆ alkyl, and each R⁷ is independently C₁-C₆ alkoxy;
   each R⁶ is independently C₁-C₆ alkyl, and each R⁷ is independently C₁-C₆ alkoxy substituted with one or more halo;
   each R⁶ is independently halo, and each R⁷ is independently C₁-C₆ haloalkyl;
   each R⁶ is independently halo, and each R⁷ is independently C₁-C₆ haloalkoxy;
   each R⁶ is independently C₁-C₆ alkoxy; and each R⁷ is independently halo;
   each R⁶ is independently C₁-C₆ alkoxy; and each R⁷ is chloro;
   each R⁷ is independently C₁-C₆ alkyl, and each R⁶ is independently C₁-C₆ alkyl optionally substituted with one or more halo;
   each R⁷ is independently C₁-C₆ alkyl, and each R⁶ is independently C₁-C₆ alkyl substituted with one or more halo;
   each R⁷ is independently C₁-C₆ alkyl, and each R⁶ is independently C₃-C₇ cycloalkyl;
   each R⁷ is independently C₁-C₆ alkyl, and each R⁶ is independently halo;
   each R⁷ is independently C₁-C₆ alkyl and each R⁶ is independently halo;
   each R⁷ is independently C₁-C₆ alkyl, and each R⁶ is cyano;
   each R⁷ is independently C₃-C₇ cycloalkyl, and each R⁶ is independently C₃-C₇ cycloalkyl;
   each R⁷ is independently C₃-C₇ cycloalkyl, and each R⁶ is independently halo;
   each R⁷ is independently C₃-C₇ cycloalkyl and each R⁶ is independently halo;
   each R⁷ is independently C₁-C₆ alkyl, and each R⁶ is independently C₁-C₆ alkoxy optionally substituted with one or more halo;
   each R⁷ is independently C₁-C₆ alkyl, and each R⁶ is independently C₁-C₆ alkoxy;
   each R⁷ is independently C₁-C₆ alkyl, and each R⁶ is independently C₁-C₆ alkoxy substituted with one or more halo;
   each R⁷ is independently halo, and each R⁶ is independently C₁-C₆ haloalkyl;
   each R⁷ is independently halo, and each R⁶ is independently C₁-C₆ haloalkoxy;
   each R⁷ is independently C₁-C₆ alkoxy; and each R⁶ is independently halo;
   each R⁷ is independently C₁-C₆ alkoxy; and each R⁶ is chloro;
   Two pairs, each of one R⁶ and one R⁷, are on adjacent atoms, and each pair of one R⁶ and one R⁷ taken together with the atoms connecting them form a C₄-C₈ aliphatic carbocyclic ring;
   R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
   R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,
the substituted ring B is

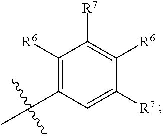

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   one R⁶ is isopropyl; the other R⁶ is trifluoromethyl; and each R⁷ is chloro;
   each R⁶ is isopropyl; one R⁷ is fluoro; and the other R⁷ is cyano;
   two pairs, each of one R⁶ and one R⁷, are on adjacent atoms, and each pair of one R⁶ and one R⁷ taken together with the atoms connecting them form a C₈ aliphatic carbocyclic ring;
   R⁶ and R⁷ on adjacent atoms taken together with the atoms connecting them form a C₄ aliphatic carbocyclic ring; and one R⁶ is chloro, fluoro, or cyano;
   R⁶ and R⁷ on adjacent atoms taken together with the atoms connecting them form a C₅ aliphatic carbocyclic ring; and one R⁶ is chloro, fluoro, or cyano;
   R⁶ and R⁷ on adjacent atoms taken together with the atoms connecting them form a C₆ aliphatic carbocyclic ring; and one R⁶ is chloro, fluoro, or cyano;
   R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
   R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
   R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,
the substituted ring B is

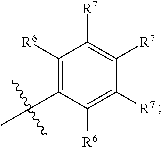

and R⁶ and R⁷ are one of the following combinations:
   each R⁶ is independently C₁-C₆ alkyl, and each R⁷ is C₁-C₆ alkyl optionally substituted with one or more halo;
   each R⁶ is independently C₁-C₆ alkyl and R⁷ is C₁-C₆ alkyl;
   each R⁶ is independently C₁-C₆ alkyl, and R⁷ is C₁-C₆ alkyl substituted with one or more halo;
   each R⁶ is independently C₁-C₆ alkyl, and R⁷ is C₃-C₇ cycloalkyl;
   each R⁶ is independently C₁-C₆ alkyl, and R⁷ is halo;
   each R⁶ is independently C₁-C₆ alkyl, and R⁷ is cyano;
   each R⁶ is independently C₃-C₇ cycloalkyl, and R⁷ is C₃-C₇ cycloalkyl;
   each R⁶ is independently C₃-C₇ cycloalkyl, and R⁷ is halo;
   each R⁶ is independently cyclopropyl and R⁷ is halo;
   each R⁶ is independently C₁-C₆ alkyl, and R⁷ is C₁-C₆ alkoxy optionally substituted with one or more halo;
   each R⁶ is independently C₁-C₆ alkyl, and R⁷ is C₁-C₆ alkoxy;
   each R⁶ is independently C₁-C₆ alkyl, and R⁷ is C₁-C₆ alkoxy substituted with one or more halo;
   each R⁶ is independently halo, and R⁷ is C₁-C₆ haloalkyl;
   each R⁶ is independently halo, and R⁷ is C₁-C₆ haloalkoxy;
   each R⁶ is independently C₁-C₆ alkoxy; and R⁷ is halo;
   each R⁶ is independently C₁-C₆ alkoxy; and R⁷ is chloro;
   R⁷ is C₁-C₆ alkyl, and each R⁶ is independently C₁-C₆ alkyl optionally substituted with one or more halo;
   R⁷ is C₁-C₆ alkyl, and each R⁶ is independently C₁-C₆ alkyl substituted with one or more halo;
   R⁷ is C₁-C₆ alkyl, and each R⁶ is independently C₃-C₇ cycloalkyl;
   R⁷ is C₁-C₆ alkyl, and each R⁶ is independently halo;
   R⁷ is C₁-C₆ alkyl and each R⁶ is independently halo;
   R⁷ is C₁-C₆ alkyl, and R⁶ is cyano;
   R⁷ is C₃-C₇ cycloalkyl, and each R⁶ is independently C₃-C₇ cycloalkyl;
   R⁷ is C₃-C₇ cycloalkyl, and each R⁶ is independently halo;
   R⁷ is C₃-C₇ cycloalkyl and each R⁶ is independently halo;
   R⁷ is C₁-C₆ alkyl, and each R⁶ is independently C₁-C₆ alkoxy optionally substituted with one or more halo;
   R⁷ is C₁-C₆ alkyl, and each R⁶ is independently C₁-C₆ alkoxy;
   R⁷ is C₁-C₆ alkyl, and each R⁶ is independently C₁-C₆ alkoxy substituted with one or more halo;
   R⁷ is halo, and each R⁶ is independently C₁-C₆ haloalkyl;
   R⁷ is halo, and each R⁶ is independently C₁-C₆ haloalkoxy;
   R⁷ is C₁-C₆ alkoxy; and each R⁶ is independently halo;
   R⁷ is C₁-C₆ alkoxy; and R⁶ is chloro;
   two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is halo and each pair of one R⁶ and one R⁷ taken together with the atoms connecting them form a C₄-C₆ aliphatic carbocyclic ring optionally substituted with one or more hydroxy, oxo, or C₁-C₆ alkyl; and one R⁷ is halo or cyano; or
   two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁷ is halo or cyano.

In some embodiments, of the compound of formula AA,
the substituted ring B is

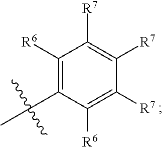

and R⁶ and R⁷ are one of the following combinations:
   each R⁶ is isopropyl; and each R⁷ is methyl;
   each R⁶ is isopropyl; and each R⁷ is isopropyl;
   each R⁶ is isopropyl; and each R⁷ is trifluoromethyl;
   each R⁶ is isopropyl; and each R⁷ is cyclopropyl;
   each R⁶ is isopropyl; and each R⁷ is chloro;
   each R⁶ is isopropyl; and each R⁷ is fluoro;
   each R⁶ is ethyl; and each R⁷ is fluoro;
   each R⁶ is isopropyl; and each R⁷ is cyano;
   each R⁶ is cyclopropyl; and each R⁷ is cyclopropyl;
   each R⁶ is cyclopropyl; and each R⁷ is chloro;
   each R⁶ is cyclopropyl; and each R⁷ is fluoro;
   each R⁶ is isopropyl; and each R⁷ is methoxy;
   each R⁶ is isopropyl; and each R⁷ is trifluoromethoxy;
   each R⁶ is chloro; and each R⁷ is trifluoromethyl;
   each R⁶ is chloro; and each R⁷ is trifluoromethoxy;
   each R⁷ is isopropyl; and each R⁶ is methyl;
   each R⁷ is isopropyl; and each R⁶ is trifluoromethyl;
   each R⁷ is isopropyl; and each R⁶ is cyclopropyl;
   each R⁷ is isopropyl; and each R⁶ is chloro;
   each R⁷ is ethyl; and each R⁶ is fluoro;
   each R⁷ is isopropyl; and each R⁶ is cyano;
   each R⁷ is cyclopropyl; and each R⁶ is cyclopropyl;
   each R⁷ is cyclopropyl; and each R⁶ is chloro;
   each R⁷ is cyclopropyl; and each R⁶ is fluoro;
   each R⁷ is isopropyl; and each R⁶ is methoxy;
   each R⁷ is isopropyl; and each R⁶ is trifluoromethoxy;
   each R⁷ is chloro; and each R⁶ is trifluoromethyl;
   each R⁷ is chloro; and each R⁶ is trifluoromethoxy;
   each R⁶ is isopropyl; two R⁷ are fluoro; and one R⁷ is chloro;
   two pairs, each of one R⁶ and one R⁷, are on adjacent atoms, and each pair of one R⁶ and one R⁷ taken together with the atoms connecting them form a C₈ aliphatic carbocyclic ring; and
   one R⁷ is chloro;
   (i) two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl; and one R⁷ is fluoro or chloro;
   (ii) two pairs, each of one R⁶ and one R⁷, are on adjacent atoms, and each pair of one R⁶ and one R⁷ taken together with the atoms connecting them form a C₈ aliphatic carbocyclic ring optionally substituted with one or more hydroxy, oxo, or methyl; and one R⁷ is fluoro or chloro;
   (iii) two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl; and one R⁷ is fluoro or chloro;
   (iv) two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; and one R⁷ is fluoro or chloro or
   two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl; and one R⁷ is fluoro or chloro.

Unless otherwise indicated, when a disclosed compound is named or depicted by a structure without specifying the stereochemistry and has one or more chiral centers, it is understood to represent all possible stereoisomers of the compound.

It is understood that the combination of variables in the formulae herein is such that the compounds are stable.

In some embodiments, provided herein is a compound that is selected from the group consisting of the compounds in Table 1:

TABLE 1

| Compound | Structure |
|---|---|
| 101 | 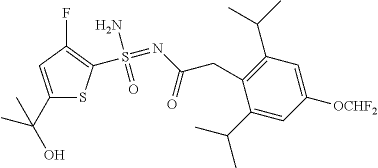 [SMILES: CC(C)c1cc(OC(F)F)cc(C(C)C)c1CC(=O)N=S(N)(=O)c1sc(C(C)(C)O)cc1F] |
| 101a | 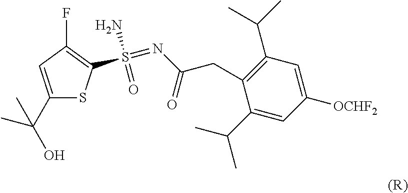 [SMILES: CC(C)c1cc(OC(F)F)cc(C(C)C)c1CC(=O)N=[S@@](N)(=O)c1sc(C(C)(C)O)cc1F ; "(R)"] |
| 101b | 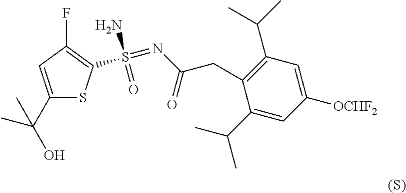 [SMILES: CC(C)c1cc(OC(F)F)cc(C(C)C)c1CC(=O)N=[S@](N)(=O)c1sc(C(C)(C)O)cc1F ; "(S)"] |
| 102 | 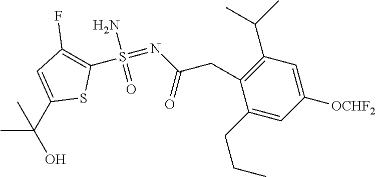 [SMILES: CCCc1cc(OC(F)F)cc(C(C)C)c1CC(=O)N=S(N)(=O)c1sc(C(C)(C)O)cc1F] |
| 103 | 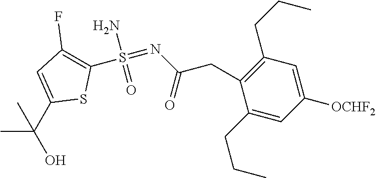 [SMILES: CCCc1cc(OC(F)F)cc(CCC)c1CC(=O)N=S(N)(=O)c1sc(C(C)(C)O)cc1F] |
| 104 | 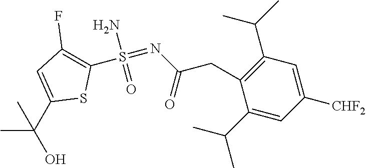 [SMILES: CC(C)c1cc(C(F)F)cc(C(C)C)c1CC(=O)N=S(N)(=O)c1sc(C(C)(C)O)cc1F] |
| 104a | 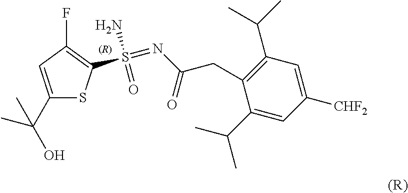 |
| 104b | 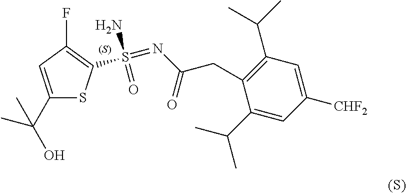 |
| 105 | 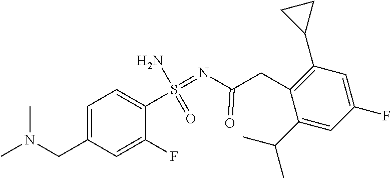 [SMILES: CC(C)c1cc(F)cc(C2CC2)c1CC(=O)N=S(N)(=O)c1ccc(CN(C)C)cc1F] |
| 106 | 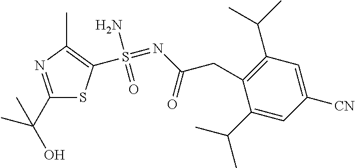 [SMILES: Cc1nc(C(C)(C)O)sc1S(N)(=O)=NC(=O)Cc1c(C(C)C)cc(C#N)cc1C(C)C] |
| 106a | 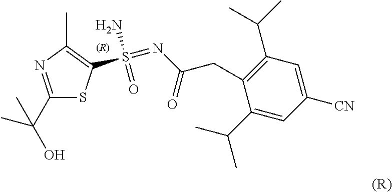 |
| 106b | 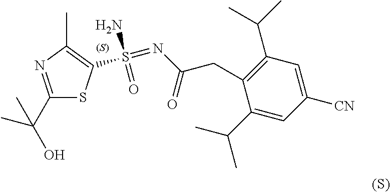 |
| 107 | 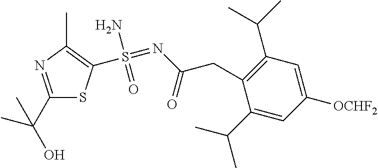 [SMILES: Cc1nc(C(C)(C)O)sc1S(N)(=O)=NC(=O)Cc1c(C(C)C)cc(OC(F)F)cc1C(C)C] |
| 107a | 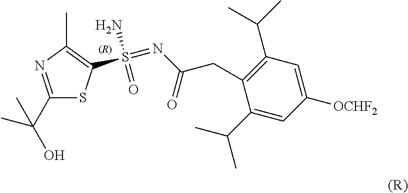 |
| 107b | 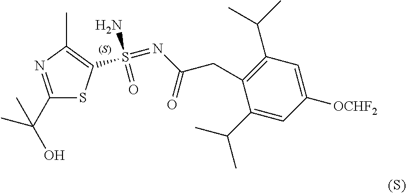 |
| 108 | 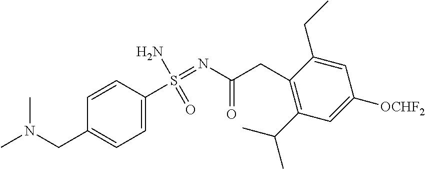 [SMILES: CCc1cc(OC(F)F)cc(C(C)C)c1CC(=O)N=S(N)(=O)c1ccc(CN(C)C)cc1] |
| 109 | 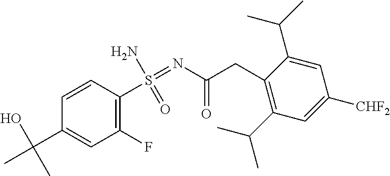 [SMILES: CC(C)c1cc(C(F)F)cc(C(C)C)c1CC(=O)N=S(N)(=O)c1ccc(C(C)(C)O)cc1F] |
| 110 | 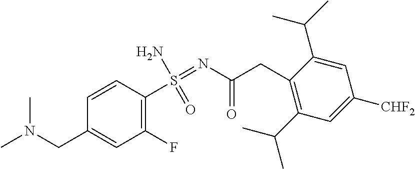 [SMILES: CC(C)c1cc(C(F)F)cc(C(C)C)c1CC(=O)N=S(N)(=O)c1ccc(CN(C)C)cc1F] |
| 110a′ | 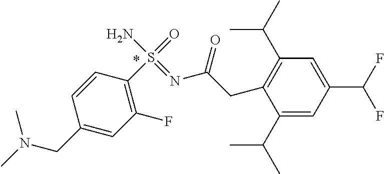 [SMILES: CC(C)c1cc(C(F)F)cc(C(C)C)c1CC(=O)N=S(N)(=O)c1ccc(CN(C)C)cc1F] |
| 110b′ | 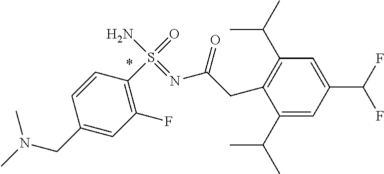 [SMILES: CC(C)c1cc(C(F)F)cc(C(C)C)c1CC(=O)N=S(N)(=O)c1ccc(CN(C)C)cc1F] |
| 110a | 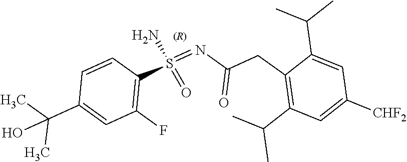 |
| 110b | 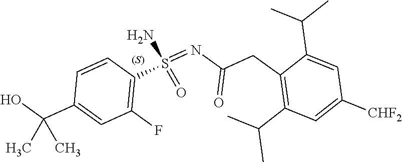 |
| 111 | 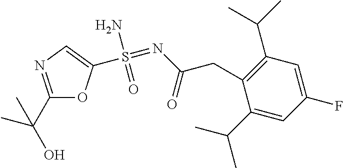 [SMILES: CC(C)c1cc(F)cc(C(C)C)c1CC(=O)N=S(N)(=O)c1cnc(C(C)(C)O)o1] |
| 112 | 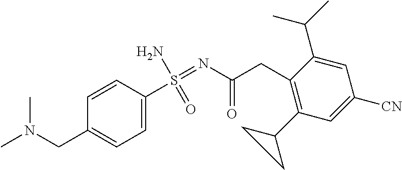 [SMILES: CC(C)c1cc(C#N)cc(C2CC2)c1CC(=O)N=S(N)(=O)c1ccc(CN(C)C)cc1] |
| 112a | 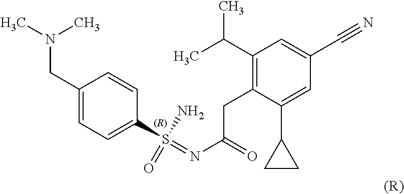 |
| 112b | 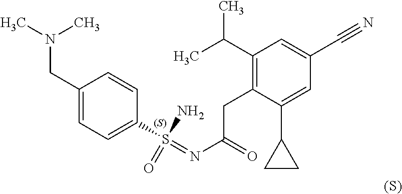 |
| 113 | 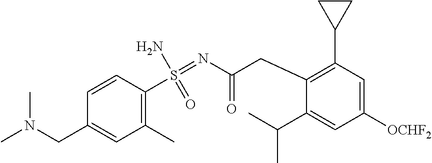 [SMILES: Cc1cc(CN(C)C)ccc1S(N)(=O)=NC(=O)Cc1c(C(C)C)cc(OC(F)F)cc1C1CC1] |
| 114 | 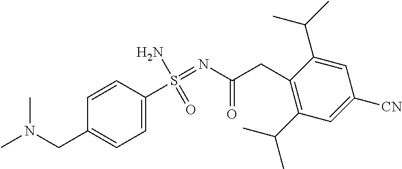 [SMILES: CC(C)c1cc(C#N)cc(C(C)C)c1CC(=O)N=S(N)(=O)c1ccc(CN(C)C)cc1] |
| 114a | 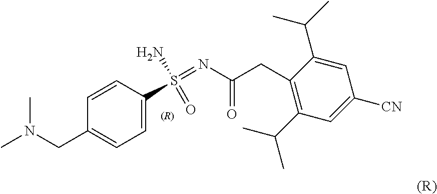 |
| 114b | 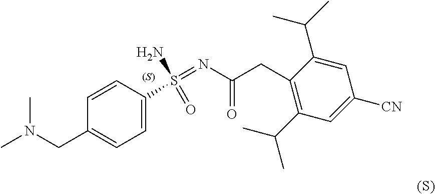 |
| 115 | 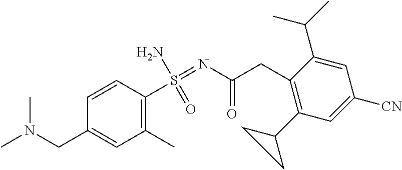 [SMILES: Cc1cc(CN(C)C)ccc1S(N)(=O)=NC(=O)Cc1c(C(C)C)cc(C#N)cc1C1CC1] |
| 116 | 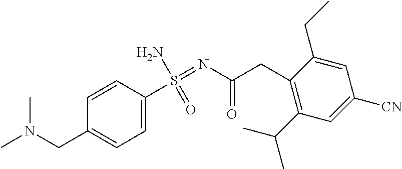 [SMILES: CCc1cc(C#N)cc(C(C)C)c1CC(=O)N=S(N)(=O)c1ccc(CN(C)C)cc1] |
| 116a | 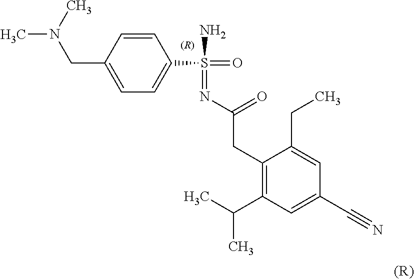 |
| 116b | 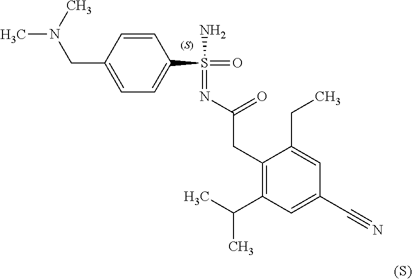 |
| 117 | 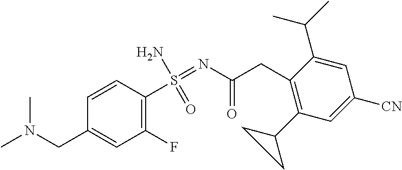 [SMILES: CC(C)c1cc(C#N)cc(C2CC2)c1CC(=O)N=S(N)(=O)c1ccc(CN(C)C)cc1F] |
| 117a | 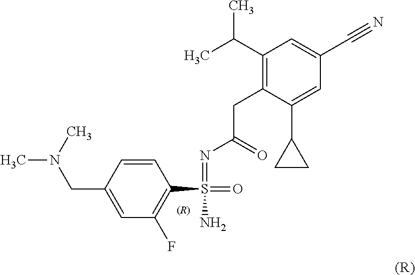 |
| 117b | 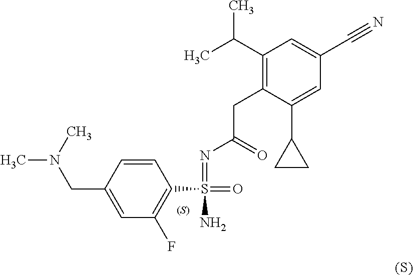 |
| 118 | 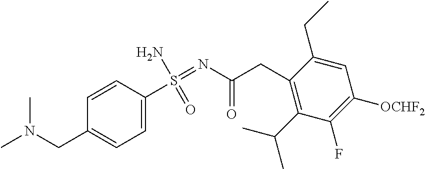 [SMILES: CCc1cc(OC(F)F)c(F)c(C(C)C)c1CC(=O)N=S(N)(=O)c1ccc(CN(C)C)cc1] |
| 119 | 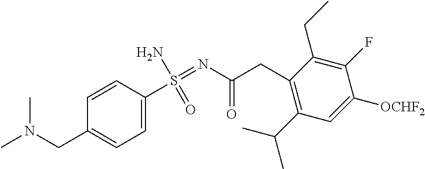 [SMILES: CCc1c(F)c(OC(F)F)cc(C(C)C)c1CC(=O)N=S(N)(=O)c1ccc(CN(C)C)cc1] |
| 120 | 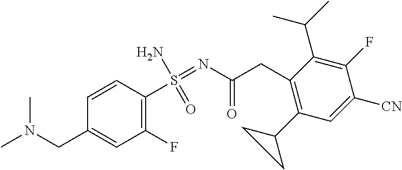 [SMILES: CC(C)c1c(F)c(C#N)cc(C2CC2)c1CC(=O)N=S(N)(=O)c1ccc(CN(C)C)cc1F] |
| 121 | 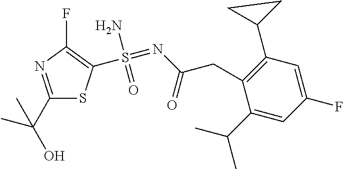 [SMILES: CC(C)c1cc(F)cc(C2CC2)c1CC(=O)N=S(N)(=O)c1sc(C(C)(C)O)nc1F] |
| 122 | 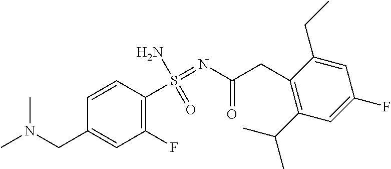 [SMILES: CCc1cc(F)cc(C(C)C)c1CC(=O)N=S(N)(=O)c1ccc(CN(C)C)cc1F] |
| 123 | 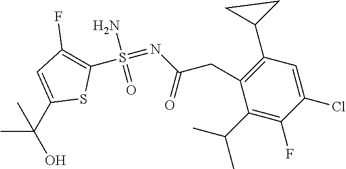 [SMILES: CC(C)c1c(F)c(Cl)cc(C2CC2)c1CC(=O)N=S(N)(=O)c1sc(C(C)(C)O)cc1F] |
| 123a | 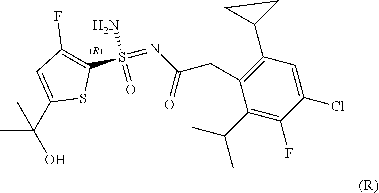 |
| 123b | 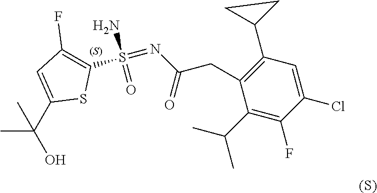 |
| 124 | 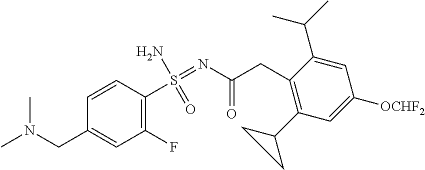 [SMILES: CC(C)c1cc(OC(F)F)cc(C2CC2)c1CC(=O)N=S(N)(=O)c1ccc(CN(C)C)cc1F] |
| 125 | 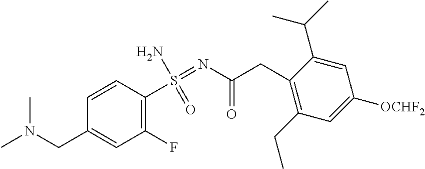 [SMILES: CCc1cc(OC(F)F)cc(C(C)C)c1CC(=O)N=S(N)(=O)c1ccc(CN(C)C)cc1F] |
| 126 | 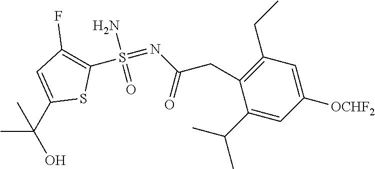 [SMILES: CCc1cc(OC(F)F)cc(C(C)C)c1CC(=O)N=S(N)(=O)c1sc(C(C)(C)O)cc1F] |
| 126a | 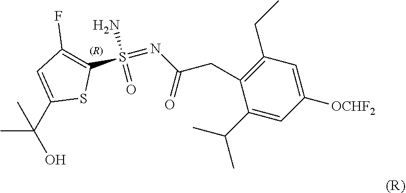 |
| 126b | 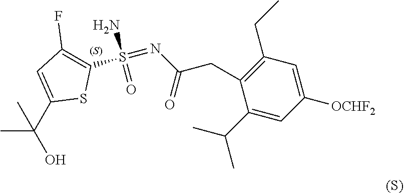 |
| 127 | 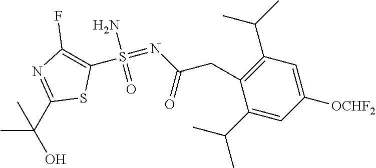 [SMILES: CC(C)c1cc(OC(F)F)cc(C(C)C)c1CC(=O)N=S(N)(=O)c1sc(C(C)(C)O)nc1F] |
| 128 | 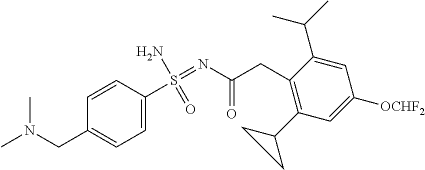 [SMILES: CC(C)c1cc(OC(F)F)cc(C2CC2)c1CC(=O)N=S(N)(=O)c1ccc(CN(C)C)cc1] |
| 129 | 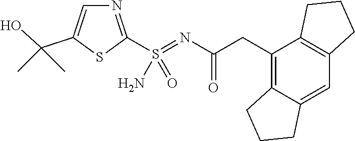 [SMILES: CC(C)(O)c1cnc(S(N)(=O)=NC(=O)Cc2c3c(cc4c2CCC4)CCC3)s1] |
| 129a | 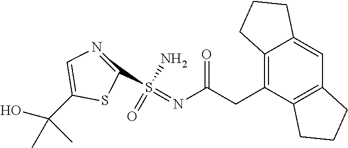 [SMILES: CC(C)(O)c1cnc([S@](N)(=O)=NC(=O)Cc2c3c(cc4c2CCC4)CCC3)s1] |
| 129b | 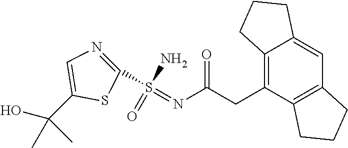 [SMILES: CC(C)(O)c1cnc([S@@](N)(=O)=NC(=O)Cc2c3c(cc4c2CCC4)CCC3)s1] |
| 130 | 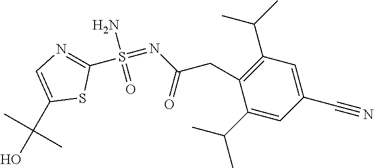 [SMILES: CC(C)c1cc(C#N)cc(C(C)C)c1CC(=O)N=S(N)(=O)c1ncc(C(C)(C)O)s1] |
| 130a | 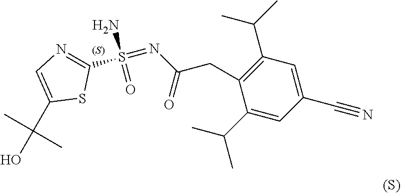 |
| 130b | 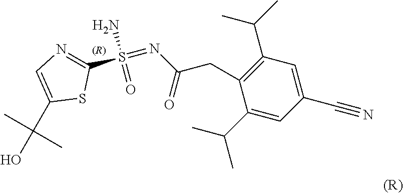 |
| 131 | 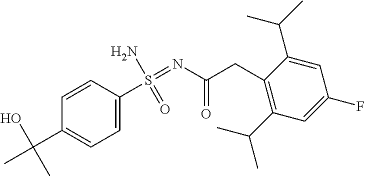 [SMILES: CC(C)c1cc(F)cc(C(C)C)c1CC(=O)N=S(N)(=O)c1ccc(C(C)(C)O)cc1] |
| 131a | 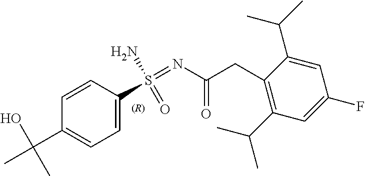 |
| 131b | 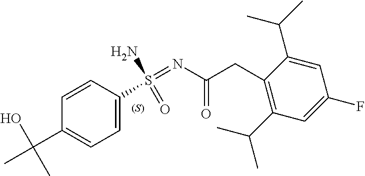 |
| 132 | 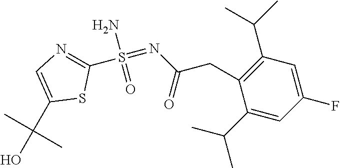 [SMILES: CC(C)c1cc(F)cc(C(C)C)c1CC(=O)N=S(N)(=O)c1ncc(C(C)(C)O)s1] |
| 132a | 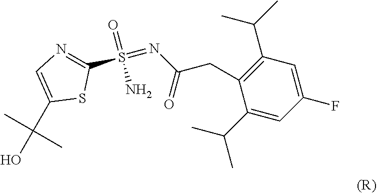 [SMILES: CC(C)c1cc(F)cc(C(C)C)c1CC(=O)N=[S@](N)(=O)c1ncc(C(C)(C)O)s1 ; "(R)"] |
| 132b | 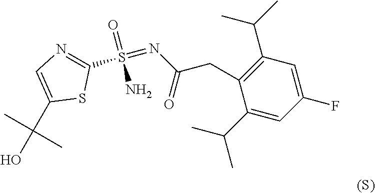 [SMILES: CC(C)c1cc(F)cc(C(C)C)c1CC(=O)N=[S@@](N)(=O)c1ncc(C(C)(C)O)s1 ; "(S)"] |
| 133 | 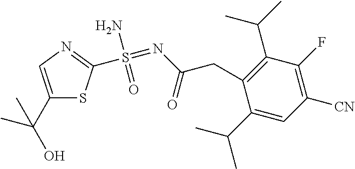 [SMILES: CC(C)c1cc(C#N)c(F)c(C(C)C)c1CC(=O)N=S(N)(=O)c1ncc(C(C)(C)O)s1] |
| 133a | 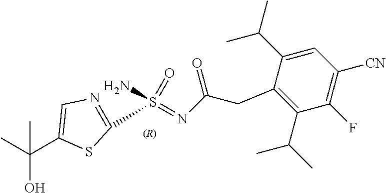 |
| 133b | 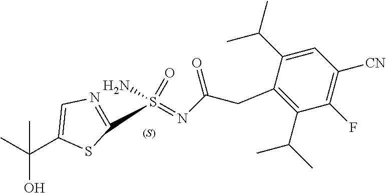 |
| 134 | 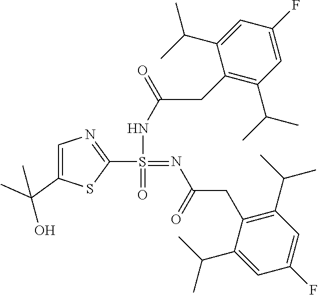 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] |
| 135 | 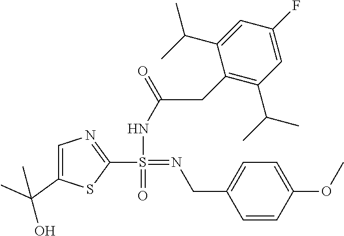 [SMILES: COc1ccc(CN=S(=O)(NC(=O)Cc2c(C(C)C)cc(F)cc2C(C)C)c2ncc(C(C)(C)O)s2)cc1] |
| 136 | 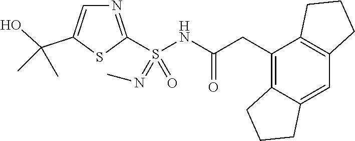 [SMILES: CN=S(=O)(NC(=O)Cc1c2c(cc3c1CCC3)CCC2)c1ncc(C(C)(C)O)s1] |
| 137 | 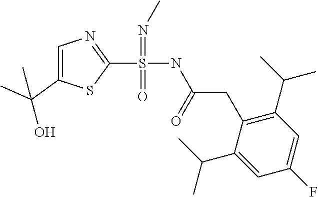 [SMILES: CN=S(=O)(NC(=O)Cc1c(C(C)C)cc(F)cc1C(C)C)c1ncc(C(C)(C)O)s1] |
| 138 | 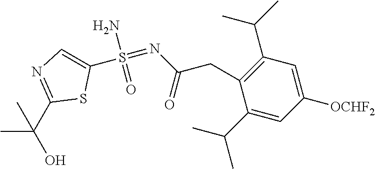 [SMILES: CC(C)c1cc(OC(F)F)cc(C(C)C)c1CC(=O)N=S(N)(=O)c1cnc(C(C)(C)O)s1] |
| 138a | 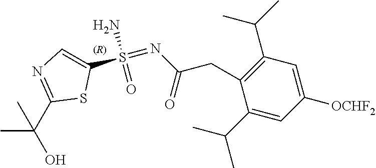 |
| 138b | 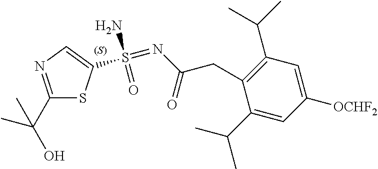 |
| 139 | 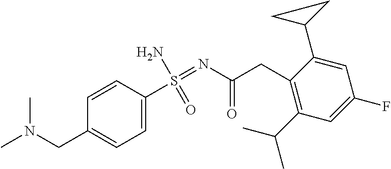 [SMILES: CC(C)c1cc(F)cc(C2CC2)c1CC(=O)N=S(N)(=O)c1ccc(CN(C)C)cc1] |
| 139a | 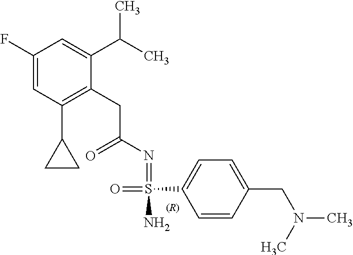 |
| 139b | 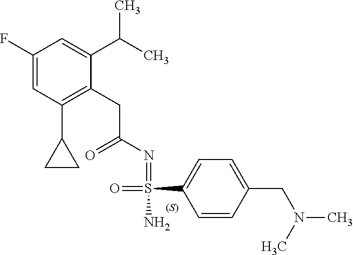 |
| 140 | 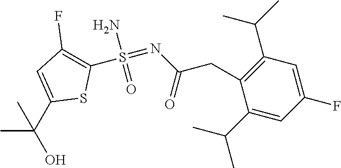 [SMILES: CC(C)c1cc(F)cc(C(C)C)c1CC(=O)N=S(N)(=O)c1sc(C(C)(C)O)cc1F] |
| 140a | 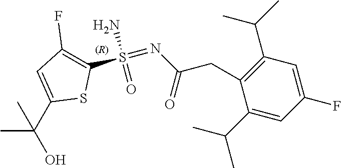 |
| 140b | 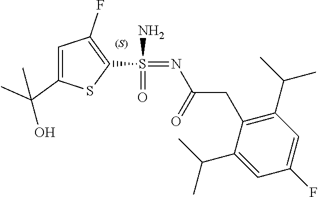 |
| 141 | 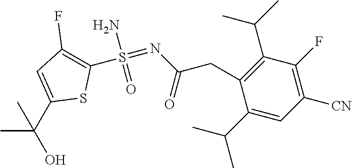 [SMILES: CC(C)c1cc(C#N)c(F)c(C(C)C)c1CC(=O)N=S(N)(=O)c1sc(C(C)(C)O)cc1F] |
| 141a | 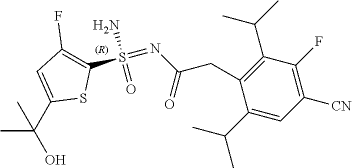 |
| 141b | 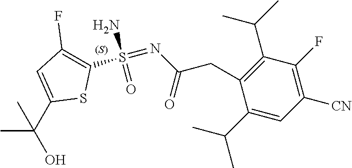 |
| 142 | 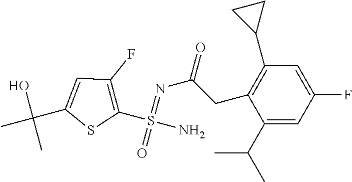 [SMILES: CC(C)c1cc(F)cc(C2CC2)c1CC(=O)N=S(N)(=O)c1sc(C(C)(C)O)cc1F] |
| 143 | 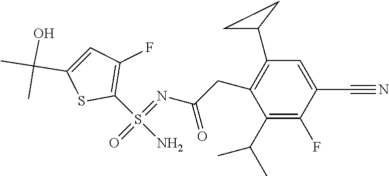 [SMILES: CC(C)c1c(F)c(C#N)cc(C2CC2)c1CC(=O)N=S(N)(=O)c1sc(C(C)(C)O)cc1F] |
| 144 | 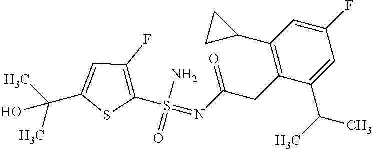 [SMILES: CC(C)c1cc(F)cc(C2CC2)c1CC(=O)N=S(N)(=O)c1sc(C(C)(C)O)cc1F] |
| 144a | 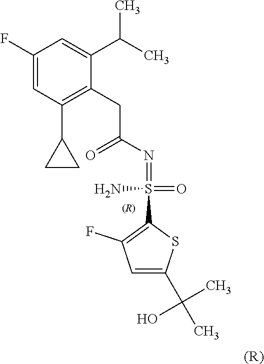 |
| 144b | 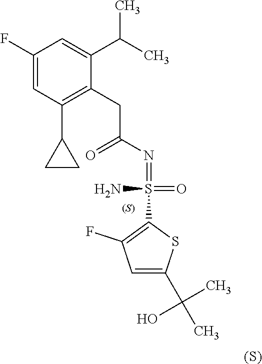 |
| 145 | 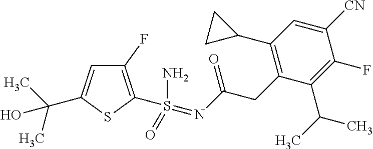 [SMILES: CC(C)c1c(F)c(C#N)cc(C2CC2)c1CC(=O)N=S(N)(=O)c1sc(C(C)(C)O)cc1F] |
| 145a | 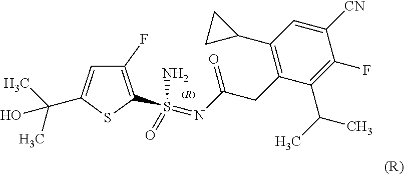 |
| 145b | 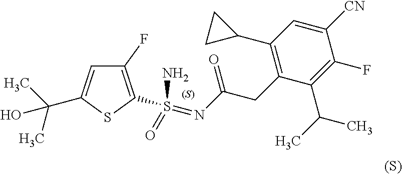 |
| 146 | 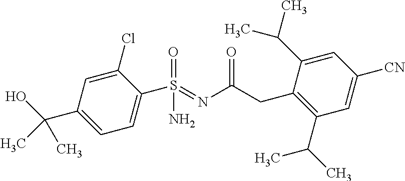 [SMILES: CC(C)c1cc(C#N)cc(C(C)C)c1CC(=O)N=S(N)(=O)c1ccc(C(C)(C)O)cc1Cl] |
| 147 | 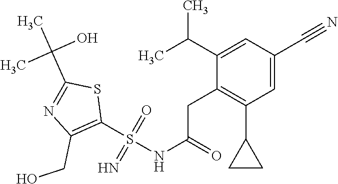 [SMILES: CC(C)c1cc(C#N)cc(C2CC2)c1CC(=O)NS(=N)(=O)c1sc(C(C)(C)O)nc1CO] |
| 147a | 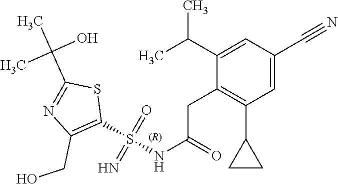 |
| 147b | 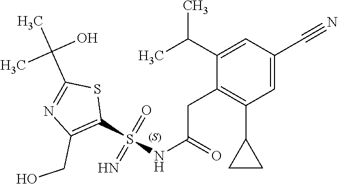 |
| 148 | 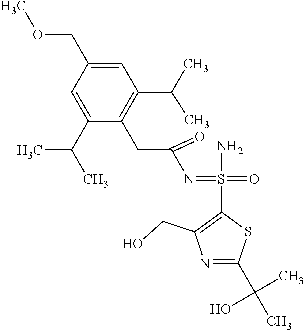 [SMILES: COCc1cc(C(C)C)c(CC(=O)N=S(N)(=O)c2sc(C(C)(C)O)nc2CO)c(C(C)C)c1] |
| 148a | 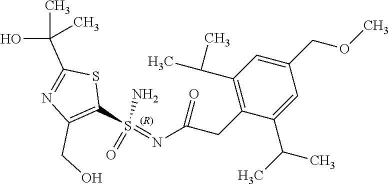 |
| 148b | 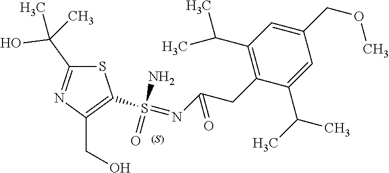 |
| 149a | 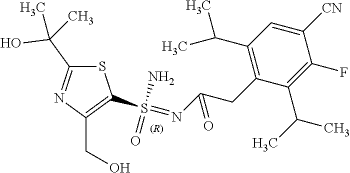 |
| 149b | 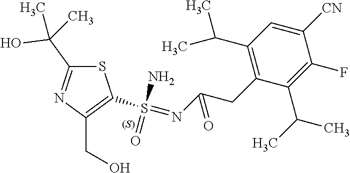 |
| 150 | 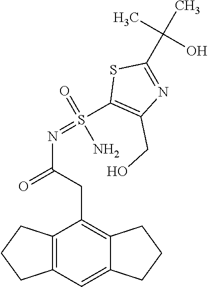 [SMILES: CC(C)(O)c1nc(CO)c(S(N)(=O)=NC(=O)Cc2c3c(cc4c2CCC4)CCC3)s1] |
| 150a | 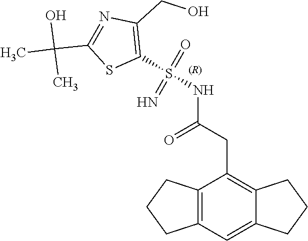 |
| 150b | 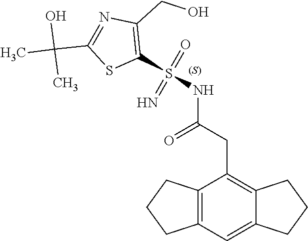 |
| 151 | 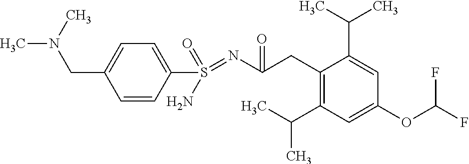 [SMILES: CC(C)c1cc(OC(F)F)cc(C(C)C)c1CC(=O)N=S(N)(=O)c1ccc(CN(C)C)cc1] |
| 152 | 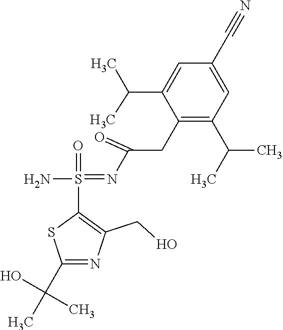 [SMILES: CC(C)c1cc(C#N)cc(C(C)C)c1CC(=O)N=S(N)(=O)c1sc(C(C)(C)O)nc1CO] |
| 152a | 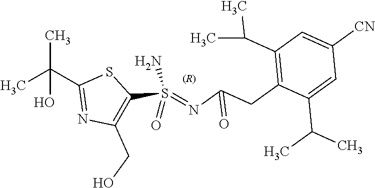 |
| 152b | 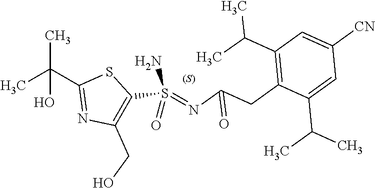 |
| 153 | 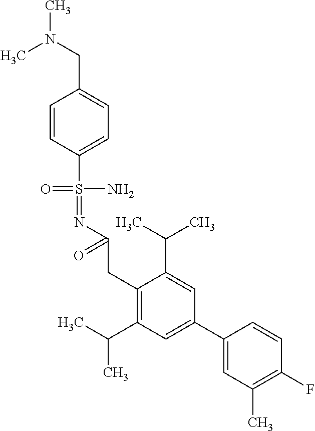 [SMILES: Cc1cc(-c2cc(C(C)C)c(CC(=O)N=S(N)(=O)c3ccc(CN(C)C)cc3)c(C(C)C)c2)ccc1F] |
| 154 | 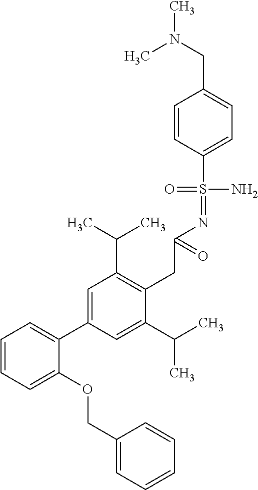 [SMILES: CC(C)c1cc(-c2ccccc2OCc2ccccc2)cc(C(C)C)c1CC(=O)N=S(N)(=O)c1ccc(CN(C)C)cc1] |
| 155 | 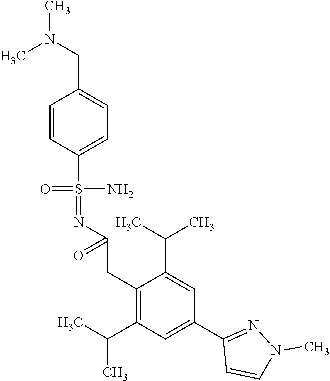 |
| 156 | 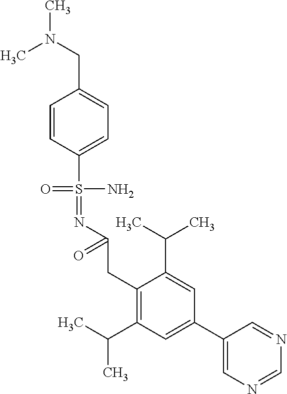 [SMILES: CC(C)c1cc(-c2cncnc2)cc(C(C)C)c1CC(=O)N=S(N)(=O)c1ccc(CN(C)C)cc1] |
| 157 | 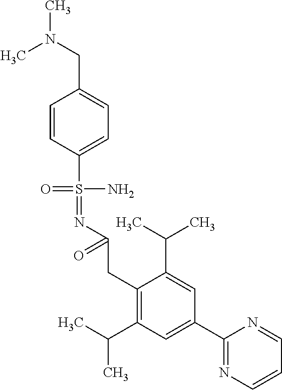 [SMILES: CC(C)c1cc(-c2ncccn2)cc(C(C)C)c1CC(=O)N=S(N)(=O)c1ccc(CN(C)C)cc1] |
| 158 | 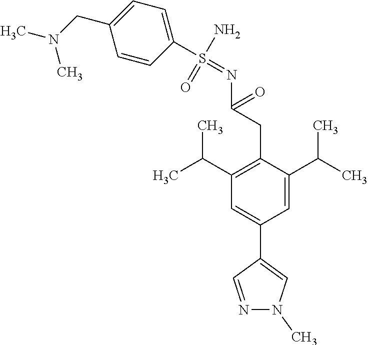 [SMILES: CC(C)c1cc(-c2cnn(C)c2)cc(C(C)C)c1CC(=O)N=S(N)(=O)c1ccc(CN(C)C)cc1] |
| 159 | 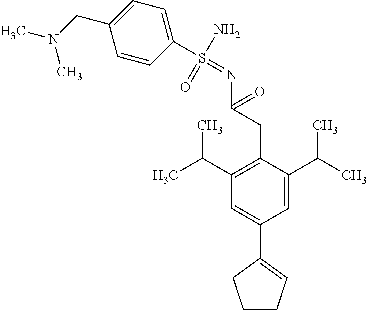 [SMILES: CC(C)c1cc(C2=CCCC2)cc(C(C)C)c1CC(=O)N=S(N)(=O)c1ccc(CN(C)C)cc1] |
| 160 | 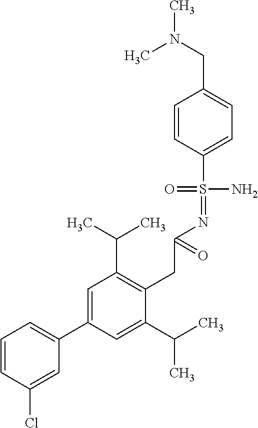 [SMILES: CC(C)c1cc(-c2cccc(Cl)c2)cc(C(C)C)c1CC(=O)N=S(N)(=O)c1ccc(CN(C)C)cc1] |
| 161 | 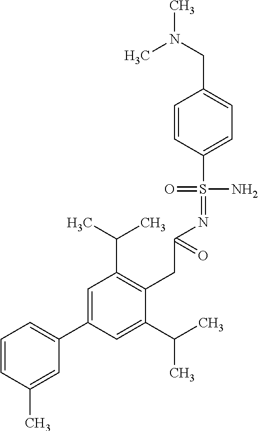 [SMILES: Cc1cccc(-c2cc(C(C)C)c(CC(=O)N=S(N)(=O)c3ccc(CN(C)C)cc3)c(C(C)C)c2)c1] |
| 162 | 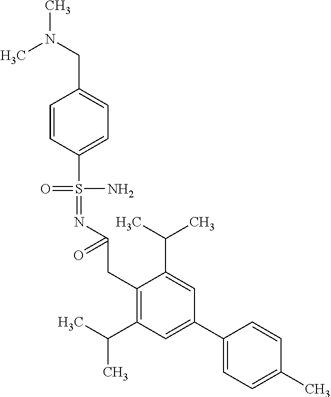 |
| 163 | 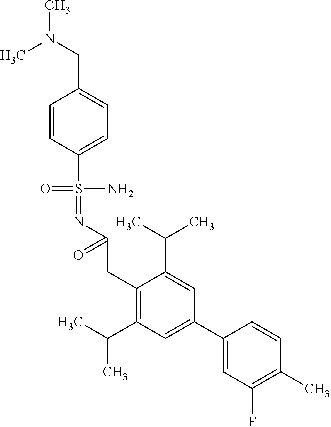 |
| 164 | 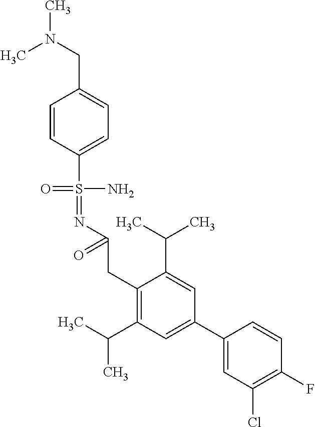 [SMILES: CC(C)c1cc(-c2ccc(F)c(Cl)c2)cc(C(C)C)c1CC(=O)N=S(N)(=O)c1ccc(CN(C)C)cc1] |
| 165 | 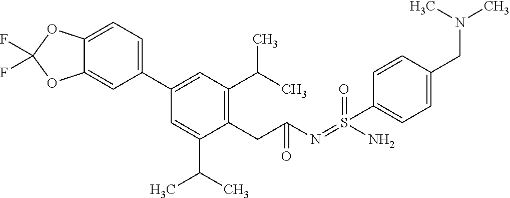 [SMILES: CC(C)c1cc(-c2ccc3c(c2)OC(F)(F)O3)cc(C(C)C)c1CC(=O)N=S(N)(=O)c1ccc(CN(C)C)cc1] |
| 166 | 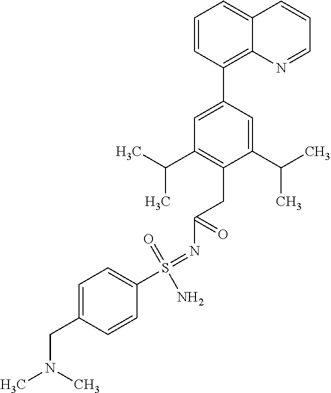 [SMILES: CC(C)c1cc(-c2cccc3cccnc23)cc(C(C)C)c1CC(=O)N=S(N)(=O)c1ccc(CN(C)C)cc1] |
| 167 | 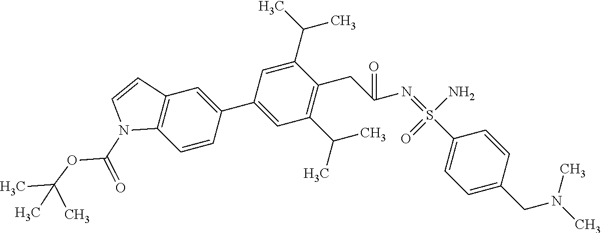 [SMILES: CC(C)c1cc(-c2ccc3c(ccn3C(=O)OC(C)(C)C)c2)cc(C(C)C)c1CC(=O)N=S(N)(=O)c1ccc(CN(C)C)cc1] |
| 168 | 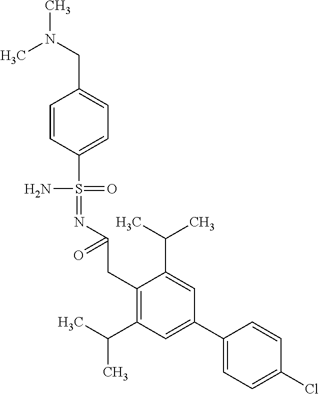 [SMILES: CC(C)c1cc(-c2ccc(Cl)cc2)cc(C(C)C)c1CC(=O)N=S(N)(=O)c1ccc(CN(C)C)cc1] |
| 169 | 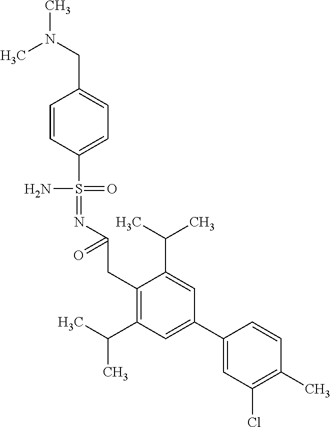 |
| 170 | 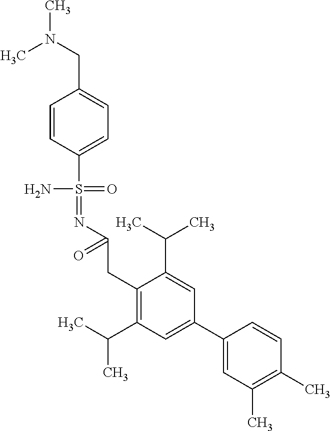 |
| 171 | 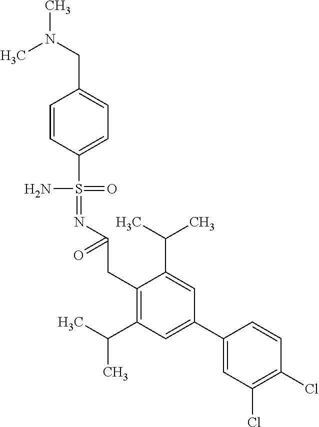 [SMILES: CC(C)c1cc(-c2ccc(Cl)c(Cl)c2)cc(C(C)C)c1CC(=O)N=S(N)(=O)c1ccc(CN(C)C)cc1] |
| 172 | 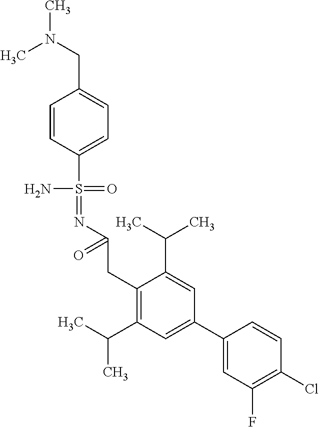 [SMILES: CC(C)c1cc(-c2ccc(Cl)c(F)c2)cc(C(C)C)c1CC(=O)N=S(N)(=O)c1ccc(CN(C)C)cc1] |
| 173 | 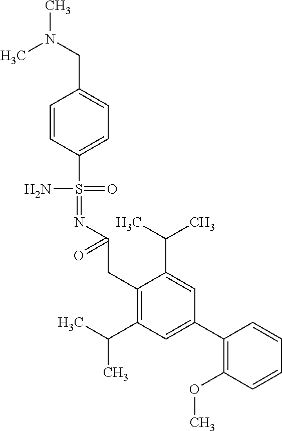 [SMILES: COc1ccccc1-c1cc(C(C)C)c(CC(=O)N=S(N)(=O)c2ccc(CN(C)C)cc2)c(C(C)C)c1] |
| 174 | 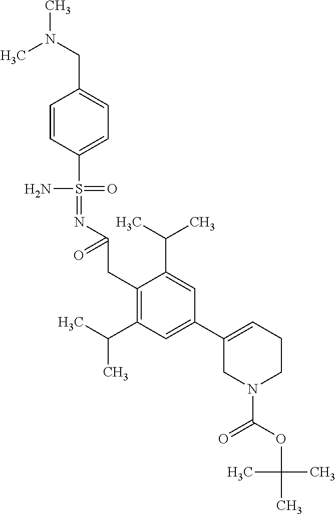 [SMILES: CC(C)c1cc(C2=CCCN(C(=O)OC(C)(C)C)C2)cc(C(C)C)c1CC(=O)N=S(N)(=O)c1ccc(CN(C)C)cc1] |
| 175 | 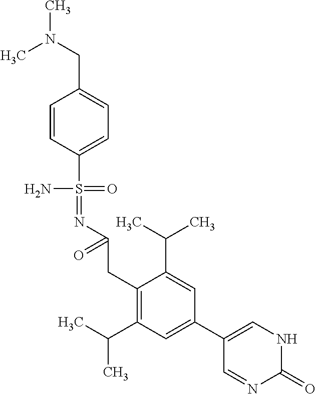 [SMILES: CC(C)c1cc(-c2cnc(=O)[nH]c2)cc(C(C)C)c1CC(=O)N=S(N)(=O)c1ccc(CN(C)C)cc1] |
| 176 | 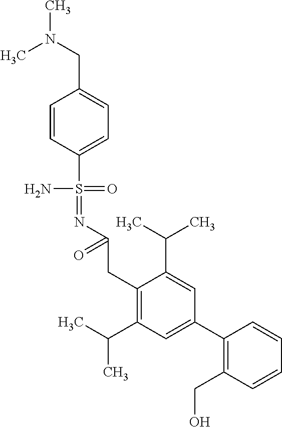 [SMILES: CC(C)c1cc(-c2ccccc2CO)cc(C(C)C)c1CC(=O)N=S(N)(=O)c1ccc(CN(C)C)cc1] |
| 177 | 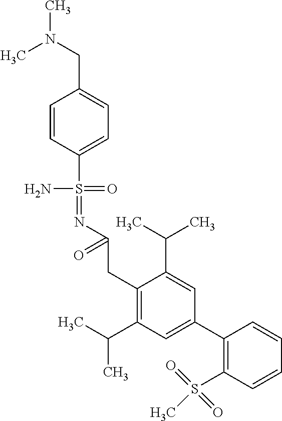 [SMILES: CC(C)c1cc(-c2ccccc2S(C)(=O)=O)cc(C(C)C)c1CC(=O)N=S(N)(=O)c1ccc(CN(C)C)cc1] |
| 178 | 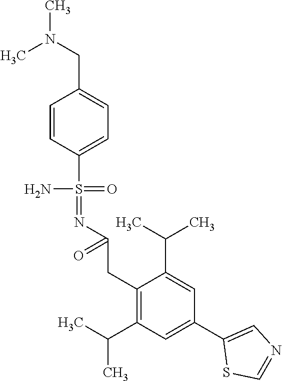 [SMILES: CC(C)c1cc(-c2cncs2)cc(C(C)C)c1CC(=O)N=S(N)(=O)c1ccc(CN(C)C)cc1] |
| 179 | 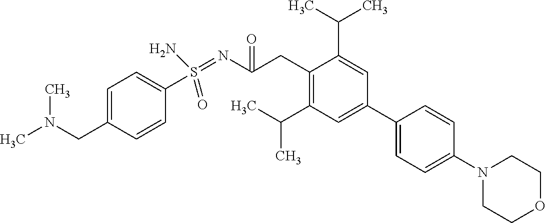 [SMILES: CC(C)c1cc(-c2ccc(N3CCOCC3)cc2)cc(C(C)C)c1CC(=O)N=S(N)(=O)c1ccc(CN(C)C)cc1] |
| 180 | 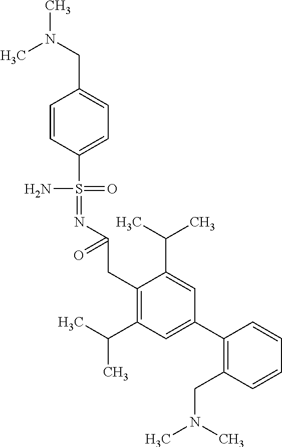 [SMILES: CC(C)c1cc(-c2ccccc2CN(C)C)cc(C(C)C)c1CC(=O)N=S(N)(=O)c1ccc(CN(C)C)cc1] |
| 181 | 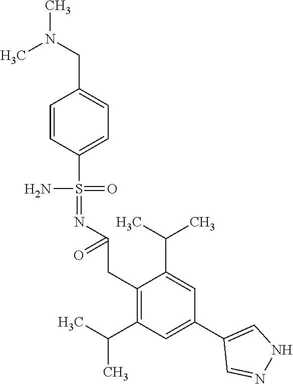 [SMILES: CC(C)c1cc(-c2cn[nH]c2)cc(C(C)C)c1CC(=O)N=S(N)(=O)c1ccc(CN(C)C)cc1] |
| 182 | 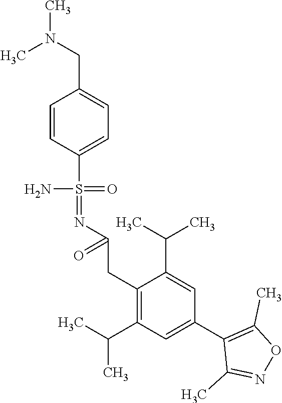 [SMILES: Cc1noc(C)c1-c1cc(C(C)C)c(CC(=O)N=S(N)(=O)c2ccc(CN(C)C)cc2)c(C(C)C)c1] |
| 183 | 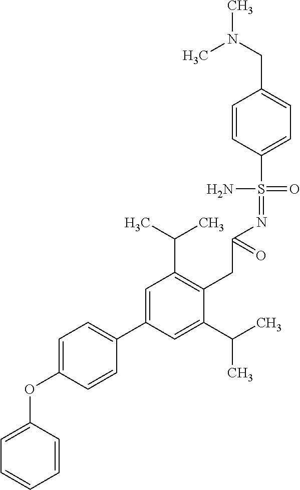 [SMILES: CC(C)c1cc(-c2ccc(Oc3ccccc3)cc2)cc(C(C)C)c1CC(=O)N=S(N)(=O)c1ccc(CN(C)C)cc1] |
| 184 | 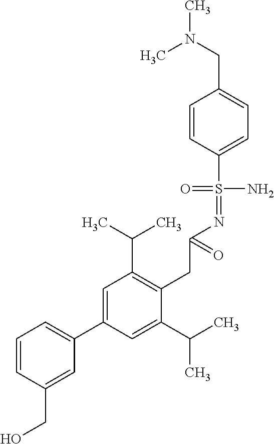 [SMILES: CC(C)c1cc(-c2cccc(CO)c2)cc(C(C)C)c1CC(=O)N=S(N)(=O)c1ccc(CN(C)C)cc1] |
| 185 | 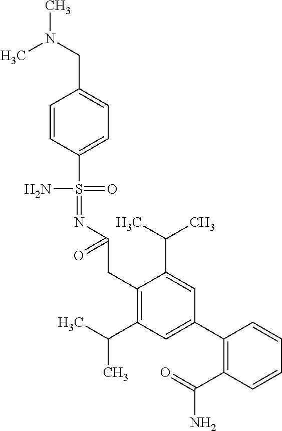 [SMILES: CC(C)c1cc(-c2ccccc2C(N)=O)cc(C(C)C)c1CC(=O)N=S(N)(=O)c1ccc(CN(C)C)cc1] |
| 186 | 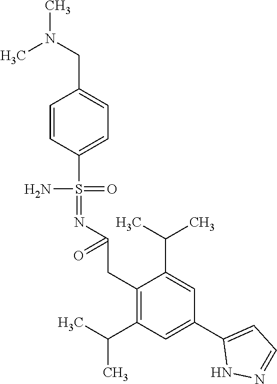 [SMILES: CC(C)c1cc(-c2ccn[nH]2)cc(C(C)C)c1CC(=O)N=S(N)(=O)c1ccc(CN(C)C)cc1] |
| 187 | 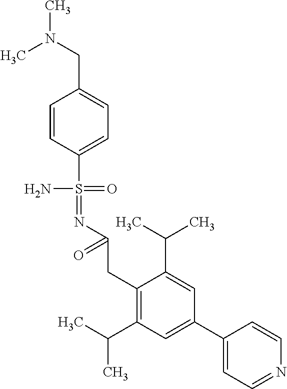 [SMILES: CC(C)c1cc(-c2ccncc2)cc(C(C)C)c1CC(=O)N=S(N)(=O)c1ccc(CN(C)C)cc1] |
| 188 | 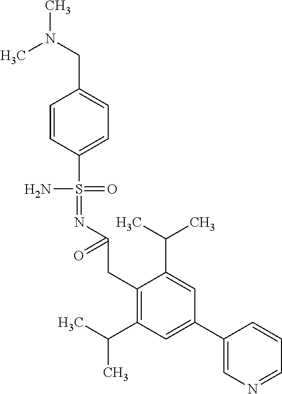 [SMILES: CC(C)c1cc(-c2cccnc2)cc(C(C)C)c1CC(=O)N=S(N)(=O)c1ccc(CN(C)C)cc1] |
| 189 | 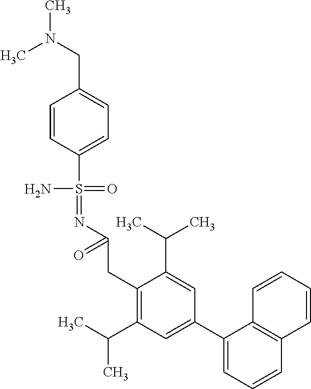 [SMILES: CC(C)c1cc(-c2cccc3ccccc23)cc(C(C)C)c1CC(=O)N=S(N)(=O)c1ccc(CN(C)C)cc1] |
| 190 | 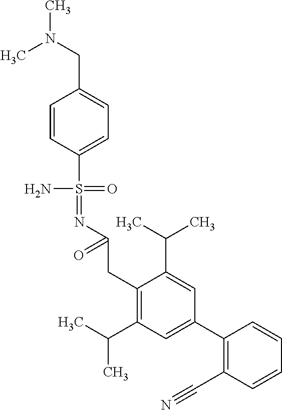 [SMILES: CC(C)c1cc(-c2ccccc2C#N)cc(C(C)C)c1CC(=O)N=S(N)(=O)c1ccc(CN(C)C)cc1] |
| 191 | 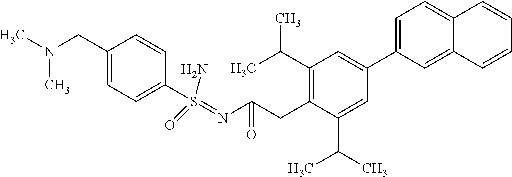 [SMILES: CC(C)c1cc(-c2ccc3ccccc3c2)cc(C(C)C)c1CC(=O)N=S(N)(=O)c1ccc(CN(C)C)cc1] |
| 192 | 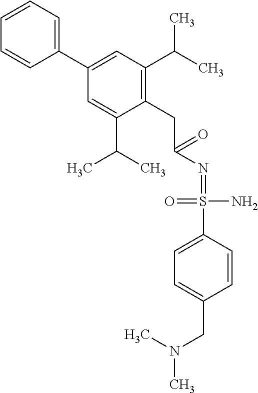 [SMILES: CC(C)c1cc(-c2ccccc2)cc(C(C)C)c1CC(=O)N=S(N)(=O)c1ccc(CN(C)C)cc1] |
| 193 | 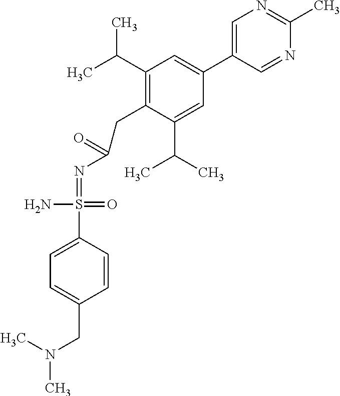 |
| 194 | 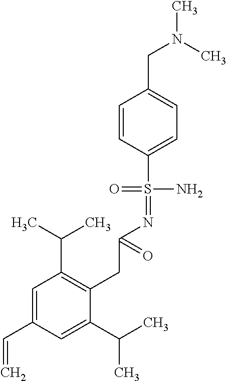 [SMILES: C=Cc1cc(C(C)C)c(CC(=O)N=S(N)(=O)c2ccc(CN(C)C)cc2)c(C(C)C)c1] |
| 195 | 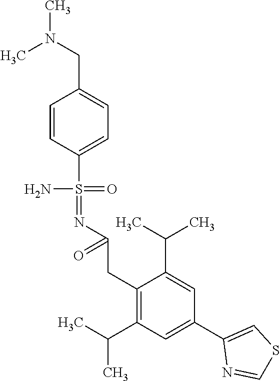 [SMILES: CC(C)c1cc(-c2cscn2)cc(C(C)C)c1CC(=O)N=S(N)(=O)c1ccc(CN(C)C)cc1] |
| 196 | 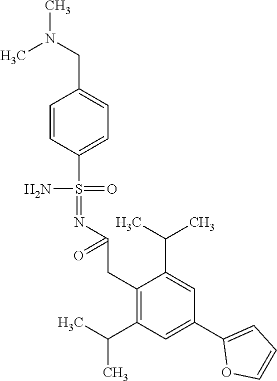 [SMILES: CC(C)c1cc(-c2ccco2)cc(C(C)C)c1CC(=O)N=S(N)(=O)c1ccc(CN(C)C)cc1] |
| 197 | 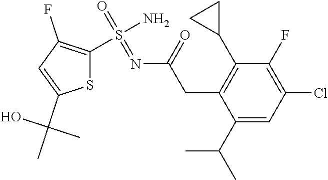 [SMILES: CC(C)c1cc(Cl)c(F)c(C2CC2)c1CC(=O)N=S(N)(=O)c1sc(C(C)(C)O)cc1F] |
| 197a | 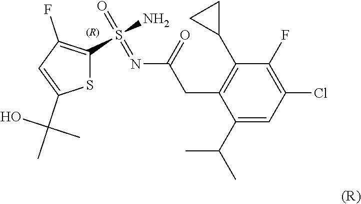 |
| 197b | 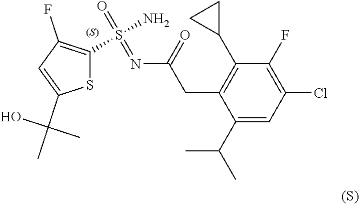 |
| 198 | 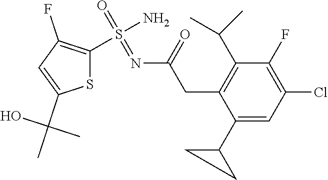 [SMILES: CC(C)c1c(F)c(Cl)cc(C2CC2)c1CC(=O)N=S(N)(=O)c1sc(C(C)(C)O)cc1F] |
| 198a | 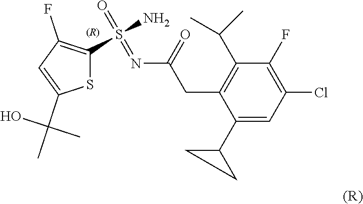 |
| 198b | 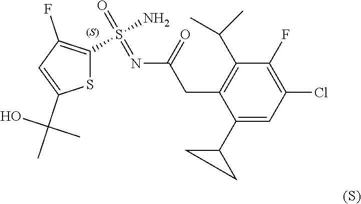 |
| 201 | 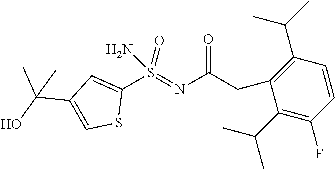 [SMILES: CC(C)c1ccc(F)c(C(C)C)c1CC(=O)N=S(N)(=O)c1cc(C(C)(C)O)cs1] |
| 201b | 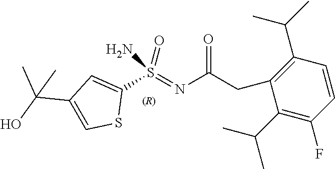 |
| 201a | 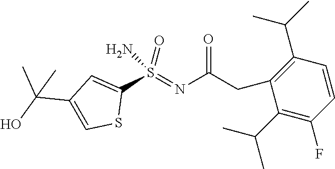 [SMILES: CC(C)c1ccc(F)c(C(C)C)c1CC(=O)N=[S@](N)(=O)c1cc(C(C)(C)O)cs1] |
| 202 | 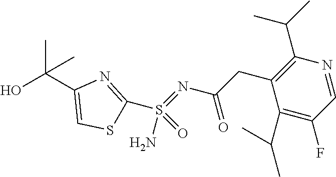 [SMILES: CC(C)c1ncc(F)c(C(C)C)c1CC(=O)N=S(N)(=O)c1nc(C(C)(C)O)cs1] |
| 202b | 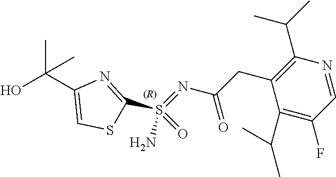 |
| 202a | 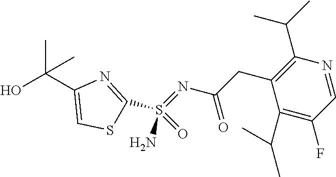 [SMILES: CC(C)c1ncc(F)c(C(C)C)c1CC(=O)N=[S@](N)(=O)c1nc(C(C)(C)O)cs1] |
| 203 | 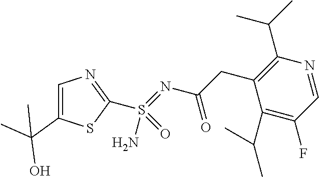 [SMILES: CC(C)c1ncc(F)c(C(C)C)c1CC(=O)N=S(N)(=O)c1ncc(C(C)(C)O)s1] |
| 203b | 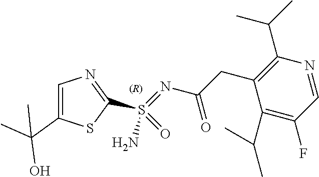 |
| 203a | 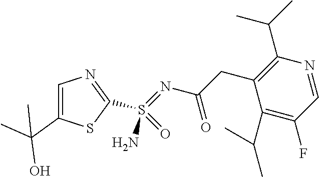 [SMILES: CC(C)c1ncc(F)c(C(C)C)c1CC(=O)N=[S@](N)(=O)c1ncc(C(C)(C)O)s1] |
| 204 | 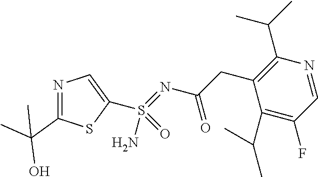 [SMILES: CC(C)c1ncc(F)c(C(C)C)c1CC(=O)N=S(N)(=O)c1cnc(C(C)(C)O)s1] |
| 204b | 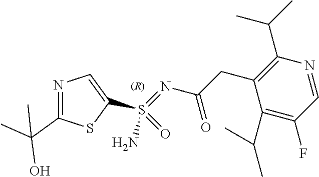 |
| 204a | 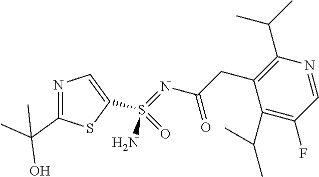 [SMILES: CC(C)c1ncc(F)c(C(C)C)c1CC(=O)N=[S@](N)(=O)c1cnc(C(C)(C)O)s1] |
| 205 | 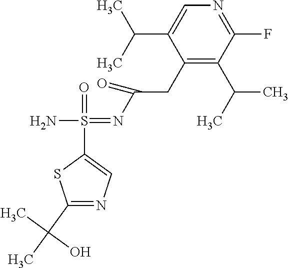 [SMILES: CC(C)c1cnc(F)c(C(C)C)c1CC(=O)N=S(N)(=O)c1cnc(C(C)(C)O)s1] |
| 206 | 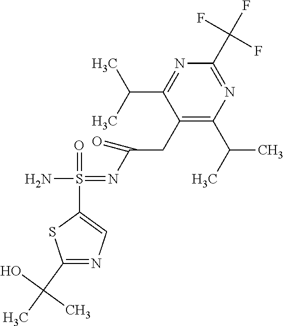 [SMILES: CC(C)c1nc(C(F)(F)F)nc(C(C)C)c1CC(=O)N=S(N)(=O)c1cnc(C(C)(C)O)s1] |
| 207 | 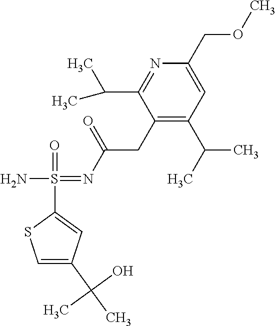 [SMILES: COCc1cc(C(C)C)c(CC(=O)N=S(N)(=O)c2cc(C(C)(C)O)cs2)c(C(C)C)n1] |
| 208 | 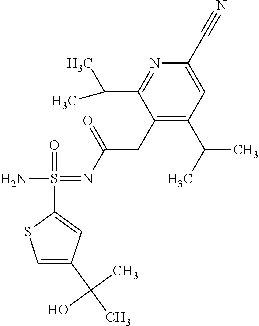 [SMILES: CC(C)c1cc(C#N)nc(C(C)C)c1CC(=O)N=S(N)(=O)c1cc(C(C)(C)O)cs1] |
| 209 | 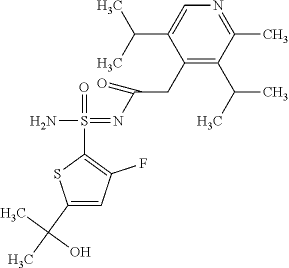 [SMILES: Cc1ncc(C(C)C)c(CC(=O)N=S(N)(=O)c2sc(C(C)(C)O)cc2F)c1C(C)C] |
| 210 | 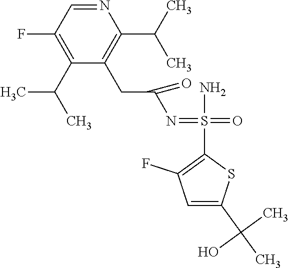 [SMILES: CC(C)c1ncc(F)c(C(C)C)c1CC(=O)N=S(N)(=O)c1sc(C(C)(C)O)cc1F] |
| 210a | 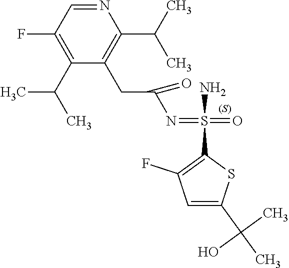 |
| 210b | 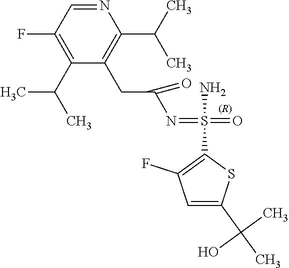 |
| 211 | 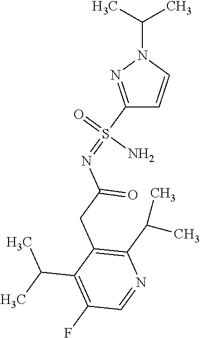 [SMILES: CC(C)c1ncc(F)c(C(C)C)c1CC(=O)N=S(N)(=O)c1ccn(C(C)C)n1] |
| 212 | 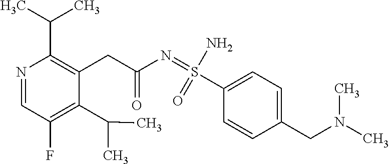 [SMILES: CC(C)c1ncc(F)c(C(C)C)c1CC(=O)N=S(N)(=O)c1ccc(CN(C)C)cc1] |
| 213 | 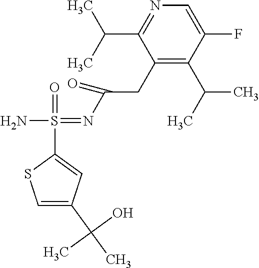 [SMILES: CC(C)c1ncc(F)c(C(C)C)c1CC(=O)N=S(N)(=O)c1cc(C(C)(C)O)cs1] |
| 213a | 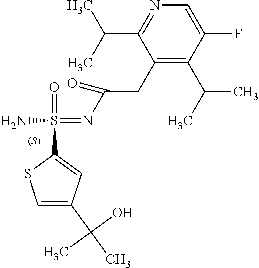 |
| 213b | 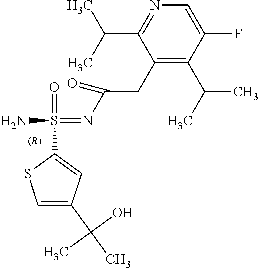 |
| 214 | 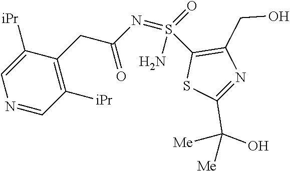 [SMILES: CC(C)c1cncc(C(C)C)c1CC(=O)N=S(N)(=O)c1sc(C(C)(C)O)nc1CO] |
| 215 | 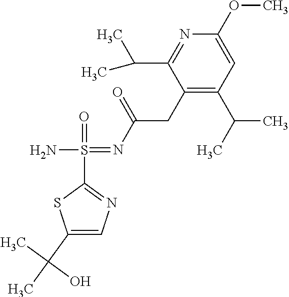 [SMILES: COc1cc(C(C)C)c(CC(=O)N=S(N)(=O)c2ncc(C(C)(C)O)s2)c(C(C)C)n1] |
| 216 | 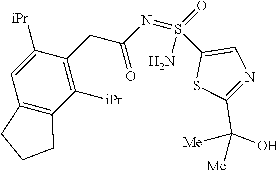 [SMILES: CC(C)c1cc2c(c(C(C)C)c1CC(=O)N=S(N)(=O)c1cnc(C(C)(C)O)s1)CCC2] |
| 216a | 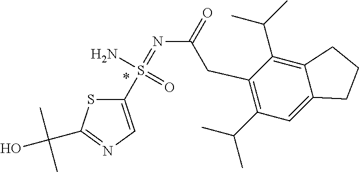 [SMILES: CC(C)c1cc2c(c(C(C)C)c1CC(=O)N=S(N)(=O)c1cnc(C(C)(C)O)s1)CCC2] |
| 216b | 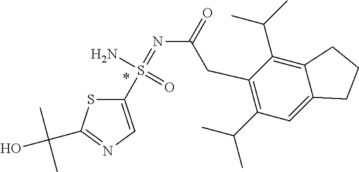 [SMILES: CC(C)c1cc2c(c(C(C)C)c1CC(=O)N=S(N)(=O)c1cnc(C(C)(C)O)s1)CCC2] |
| 217 | 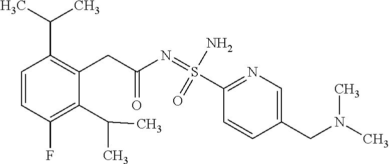 [SMILES: CC(C)c1ccc(F)c(C(C)C)c1CC(=O)N=S(N)(=O)c1ccc(CN(C)C)cn1] |
| 217a | 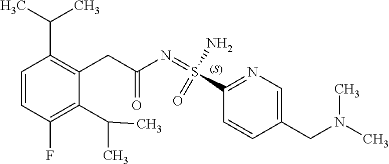 |
| 217b | 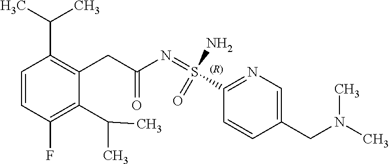 |
| 218 | 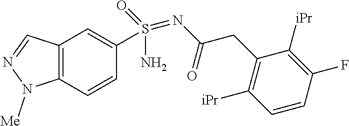 [SMILES: CC(C)c1ccc(F)c(C(C)C)c1CC(=O)N=S(N)(=O)c1ccc2c(cnn2C)c1] |
| 218a | 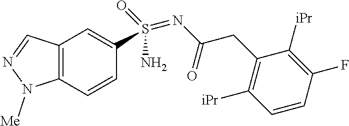 [SMILES: CC(C)c1ccc(F)c(C(C)C)c1CC(=O)N=[S@](N)(=O)c1ccc2c(cnn2C)c1] |
| 218b | 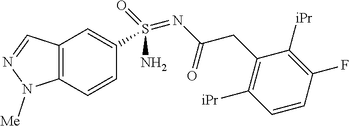 [SMILES: CC(C)c1ccc(F)c(C(C)C)c1CC(=O)N=[S@@](N)(=O)c1ccc2c(cnn2C)c1] |
| 219 | 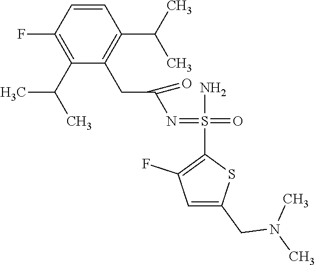 [SMILES: CC(C)c1ccc(F)c(C(C)C)c1CC(=O)N=S(N)(=O)c1sc(CN(C)C)cc1F] |
| 220 | 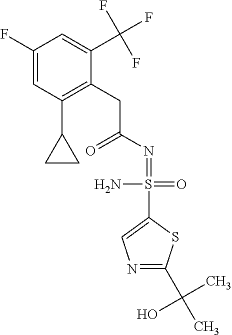 [SMILES: CC(C)(O)c1ncc(S(N)(=O)=NC(=O)Cc2c(C3CC3)cc(F)cc2C(F)(F)F)s1] |
| 220a | 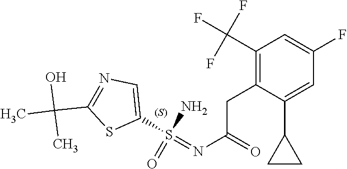 |
| 220b | 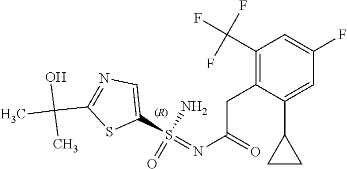 |
| 221 | 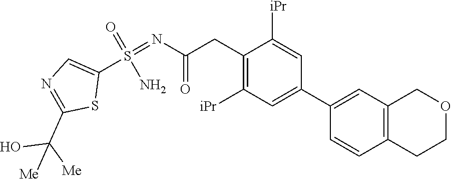 [SMILES: CC(C)c1cc(-c2ccc3c(c2)COCC3)cc(C(C)C)c1CC(=O)N=S(N)(=O)c1cnc(C(C)(C)O)s1] |
| 221a | 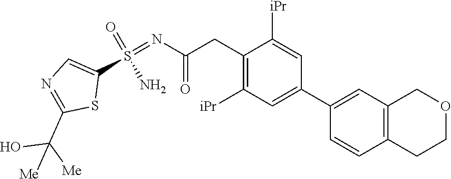 [SMILES: CC(C)c1cc(-c2ccc3c(c2)COCC3)cc(C(C)C)c1CC(=O)N=[S@](N)(=O)c1cnc(C(C)(C)O)s1] |
| 221b | 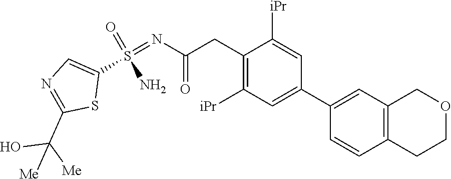 [SMILES: CC(C)c1cc(-c2ccc3c(c2)COCC3)cc(C(C)C)c1CC(=O)N=[S@@](N)(=O)c1cnc(C(C)(C)O)s1] |
| 222 | 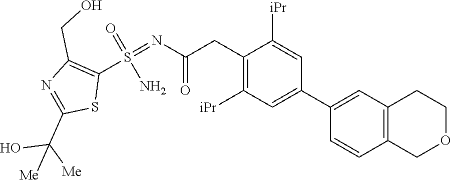 [SMILES: CC(C)c1cc(-c2ccc3c(c2)CCOC3)cc(C(C)C)c1CC(=O)N=S(N)(=O)c1sc(C(C)(C)O)nc1CO] |
| 224 | 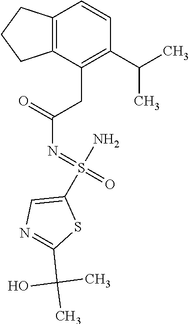 [SMILES: CC(C)c1ccc2c(c1CC(=O)N=S(N)(=O)c1cnc(C(C)(C)O)s1)CCC2] |
| 224b | 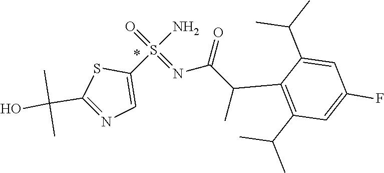 [SMILES: CC(C)c1cc(F)cc(C(C)C)c1C(C)C(=O)N=S(N)(=O)c1cnc(C(C)(C)O)s1] |
| 224ba | 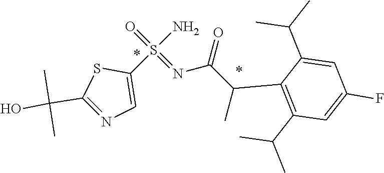 [SMILES: CC(C)c1cc(F)cc(C(C)C)c1C(C)C(=O)N=S(N)(=O)c1cnc(C(C)(C)O)s1] |
| 224aa | 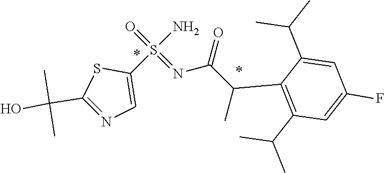 [SMILES: CC(C)c1cc(F)cc(C(C)C)c1C(C)C(=O)N=S(N)(=O)c1cnc(C(C)(C)O)s1] |
| 225 | 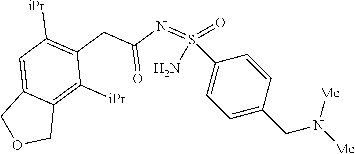 [SMILES: CC(C)c1cc2c(c(C(C)C)c1CC(=O)N=S(N)(=O)c1ccc(CN(C)C)cc1)COC2] |
| 225a | 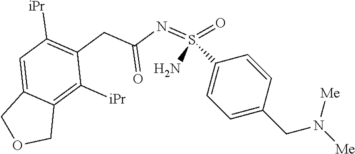 [SMILES: CC(C)c1cc2c(c(C(C)C)c1CC(=O)N=[S@](N)(=O)c1ccc(CN(C)C)cc1)COC2] |
| 225b | 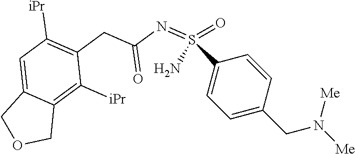 [SMILES: CC(C)c1cc2c(c(C(C)C)c1CC(=O)N=[S@@](N)(=O)c1ccc(CN(C)C)cc1)COC2] |
| 226 | 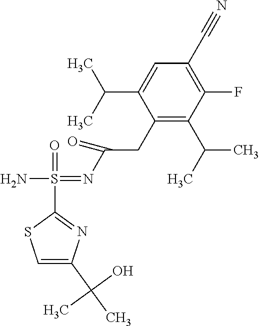 [SMILES: CC(C)c1cc(C#N)c(F)c(C(C)C)c1CC(=O)N=S(N)(=O)c1nc(C(C)(C)O)cs1] |
| 226a | 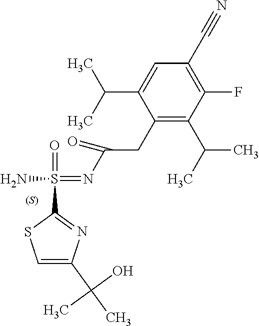 |
| 226b | 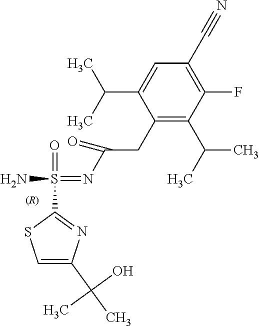 |
| 227 | 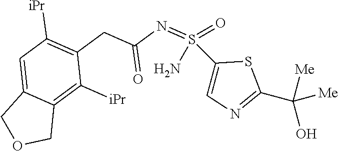 [SMILES: CC(C)c1cc2c(c(C(C)C)c1CC(=O)N=S(N)(=O)c1cnc(C(C)(C)O)s1)COC2] |
| 227a | 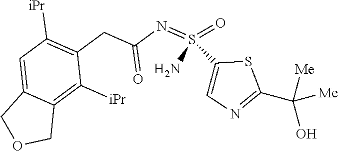 [SMILES: CC(C)c1cc2c(c(C(C)C)c1CC(=O)N=[S@](N)(=O)c1cnc(C(C)(C)O)s1)COC2] |
| 227b | 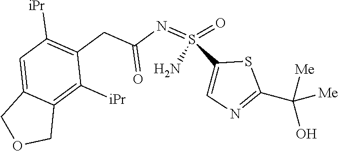 [SMILES: CC(C)c1cc2c(c(C(C)C)c1CC(=O)N=[S@@](N)(=O)c1cnc(C(C)(C)O)s1)COC2] |
| 228 | 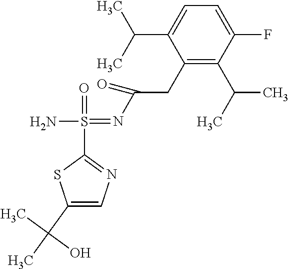 [SMILES: CC(C)c1ccc(F)c(C(C)C)c1CC(=O)N=S(N)(=O)c1ncc(C(C)(C)O)s1] |
| 228a | 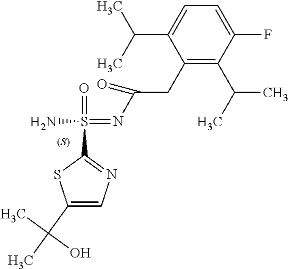 |
| 228b | 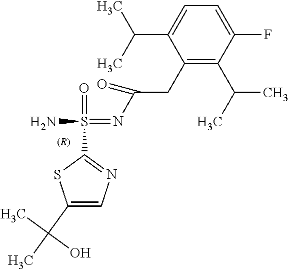 |
| 229 | 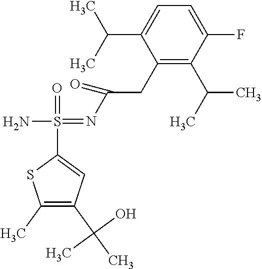 [SMILES: Cc1sc(S(N)(=O)=NC(=O)Cc2c(C(C)C)ccc(F)c2C(C)C)cc1C(C)(C)O] |
| 229a | 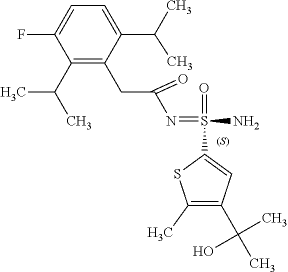 |
| 229b | 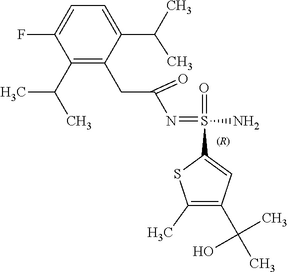 |
| 230 | 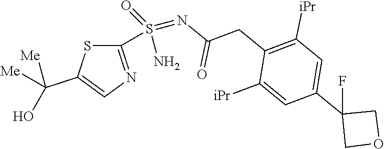 [SMILES: CC(C)c1cc(C2(F)COC2)cc(C(C)C)c1CC(=O)N=S(N)(=O)c1ncc(C(C)(C)O)s1] |
| 231 | 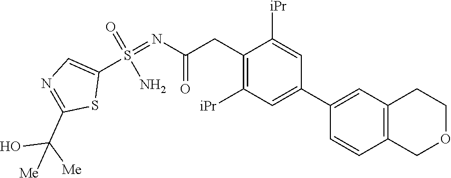 [SMILES: CC(C)c1cc(-c2ccc3c(c2)CCOC3)cc(C(C)C)c1CC(=O)N=S(N)(=O)c1cnc(C(C)(C)O)s1] |
| 232 | 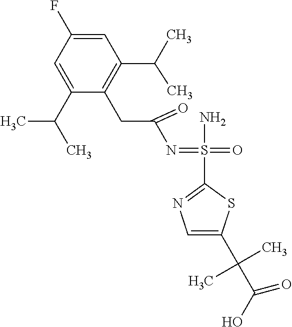 [SMILES: CC(C)c1cc(F)cc(C(C)C)c1CC(=O)N=S(N)(=O)c1ncc(C(C)(C)C(=O)O)s1] |
| 233 | 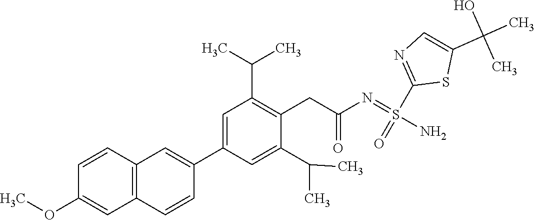 [SMILES: COc1ccc2cc(-c3cc(C(C)C)c(CC(=O)N=S(N)(=O)c4ncc(C(C)(C)O)s4)c(C(C)C)c3)ccc2c1] |
| 234 | 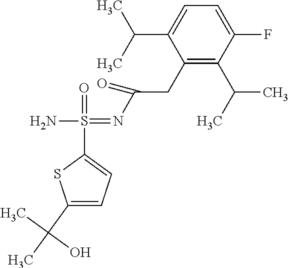 [SMILES: CC(C)c1ccc(F)c(C(C)C)c1CC(=O)N=S(N)(=O)c1ccc(C(C)(C)O)s1] |
| 234a | 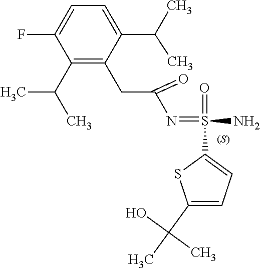 |
| 234b | 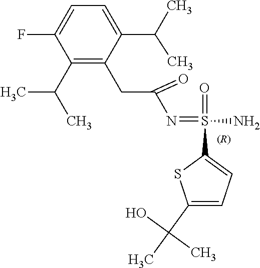 |
| 235 | 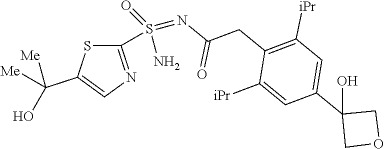 [SMILES: CC(C)c1cc(C2(O)COC2)cc(C(C)C)c1CC(=O)N=S(N)(=O)c1ncc(C(C)(C)O)s1] |
| 236 | 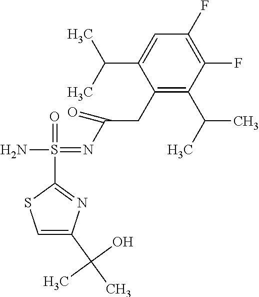 [SMILES: CC(C)c1cc(F)c(F)c(C(C)C)c1CC(=O)N=S(N)(=O)c1nc(C(C)(C)O)cs1] |
| 236a | 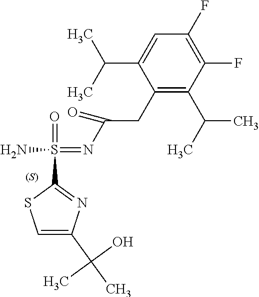 |
| 236b | 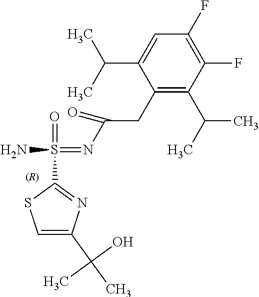 |
| 237 | 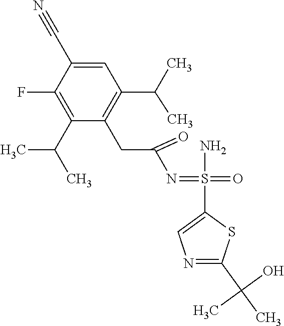 [SMILES: CC(C)c1cc(C#N)c(F)c(C(C)C)c1CC(=O)N=S(N)(=O)c1cnc(C(C)(C)O)s1] |
| 238 | 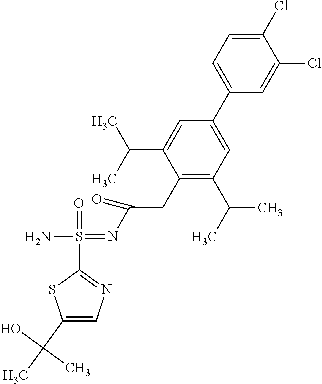 [SMILES: CC(C)c1cc(-c2ccc(Cl)c(Cl)c2)cc(C(C)C)c1CC(=O)N=S(N)(=O)c1ncc(C(C)(C)O)s1] |
| 238a | 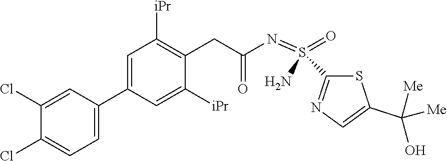 [SMILES: CC(C)c1cc(-c2ccc(Cl)c(Cl)c2)cc(C(C)C)c1CC(=O)N=[S@](N)(=O)c1ncc(C(C)(C)O)s1] |
| 238b | 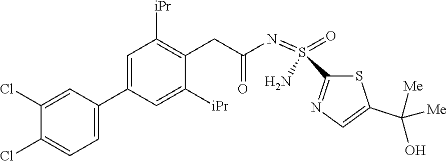 [SMILES: CC(C)c1cc(-c2ccc(Cl)c(Cl)c2)cc(C(C)C)c1CC(=O)N=[S@@](N)(=O)c1ncc(C(C)(C)O)s1] |
| 239 | 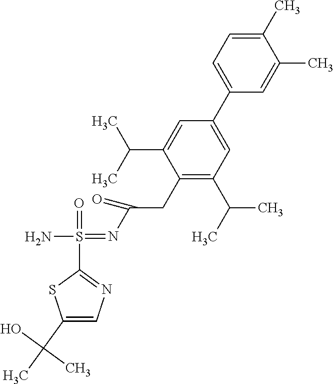 [SMILES: Cc1ccc(-c2cc(C(C)C)c(CC(=O)N=S(N)(=O)c3ncc(C(C)(C)O)s3)c(C(C)C)c2)cc1C] |
| 239a | 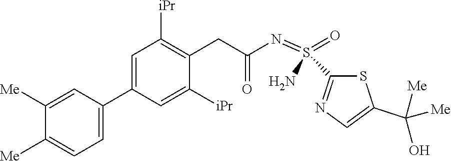 [SMILES: Cc1ccc(-c2cc(C(C)C)c(CC(=O)N=[S@](N)(=O)c3ncc(C(C)(C)O)s3)c(C(C)C)c2)cc1C] |
| 239b | 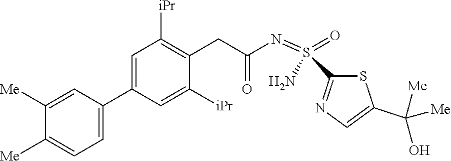 [SMILES: Cc1ccc(-c2cc(C(C)C)c(CC(=O)N=[S@@](N)(=O)c3ncc(C(C)(C)O)s3)c(C(C)C)c2)cc1C] |
| 240 | 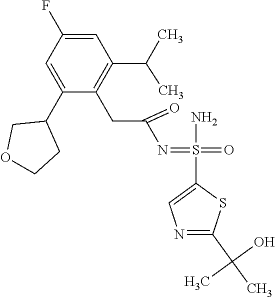 [SMILES: CC(C)c1cc(F)cc(C2CCOC2)c1CC(=O)N=S(N)(=O)c1cnc(C(C)(C)O)s1] |
| 241 | 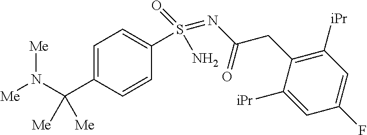 [SMILES: CC(C)c1cc(F)cc(C(C)C)c1CC(=O)N=S(N)(=O)c1ccc(C(C)(C)N(C)C)cc1] |
| 242 | 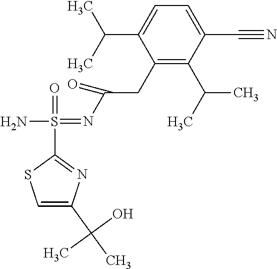 [SMILES: CC(C)c1ccc(C#N)c(C(C)C)c1CC(=O)N=S(N)(=O)c1nc(C(C)(C)O)cs1] |
| 242a | 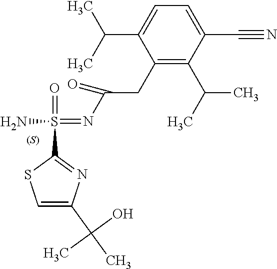 |
| 242b | 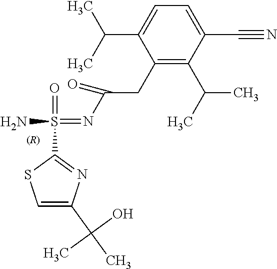 |
| 243 | 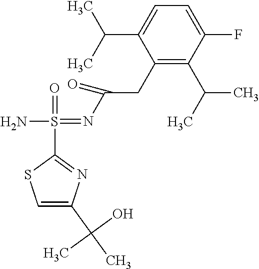 [SMILES: CC(C)c1ccc(F)c(C(C)C)c1CC(=O)N=S(N)(=O)c1nc(C(C)(C)O)cs1] |
| 243a | 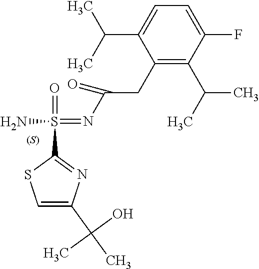 |
| 243b | 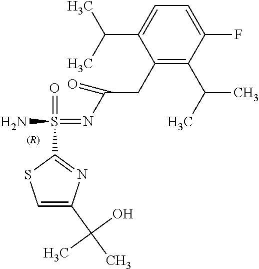 |
| 244 | 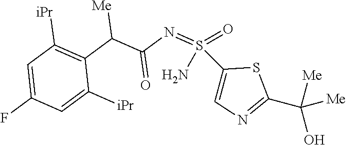 [SMILES: CC(C)c1cc(F)cc(C(C)C)c1C(C)C(=O)N=S(N)(=O)c1cnc(C(C)(C)O)s1] |
| 244aa | 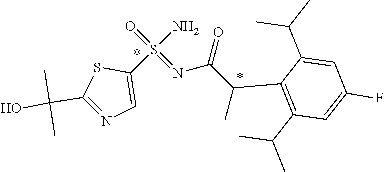 [SMILES: CC(C)c1cc(F)cc(C(C)C)c1C(C)C(=O)N=S(N)(=O)c1cnc(C(C)(C)O)s1] |
| 244b | 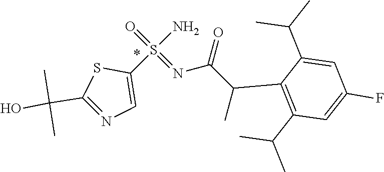 [SMILES: CC(C)c1cc(F)cc(C(C)C)c1C(C)C(=O)N=S(N)(=O)c1cnc(C(C)(C)O)s1] |
| 244ba | 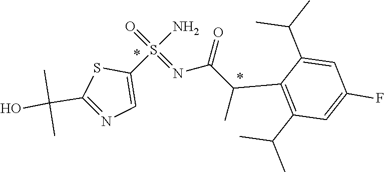 [SMILES: CC(C)c1cc(F)cc(C(C)C)c1C(C)C(=O)N=S(N)(=O)c1cnc(C(C)(C)O)s1] |
| 245 | 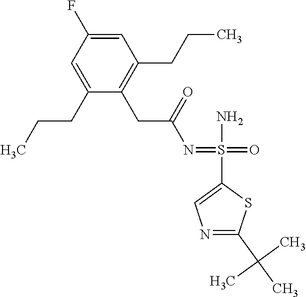 [SMILES: CCCc1cc(F)cc(CCC)c1CC(=O)N=S(N)(=O)c1cnc(C(C)(C)C)s1] |
| 246 | 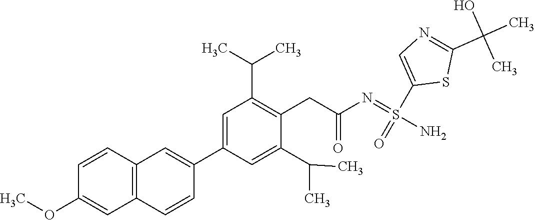 [SMILES: COc1ccc2cc(-c3cc(C(C)C)c(CC(=O)N=S(N)(=O)c4cnc(C(C)(C)O)s4)c(C(C)C)c3)ccc2c1] |
| 247 | 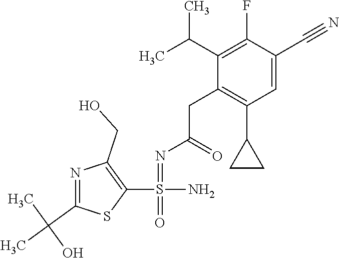 [SMILES: CC(C)c1c(F)c(C#N)cc(C2CC2)c1CC(=O)N=S(N)(=O)c1sc(C(C)(C)O)nc1CO] |
| 248 | 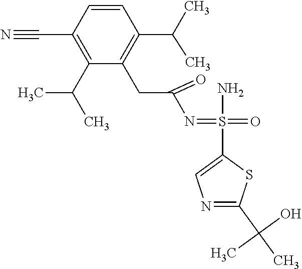 [SMILES: CC(C)c1ccc(C#N)c(C(C)C)c1CC(=O)N=S(N)(=O)c1cnc(C(C)(C)O)s1] |
| 249 | 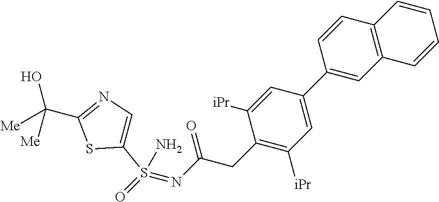 [SMILES: CC(C)c1cc(-c2ccc3ccccc3c2)cc(C(C)C)c1CC(=O)N=S(N)(=O)c1cnc(C(C)(C)O)s1] |
| 249a | 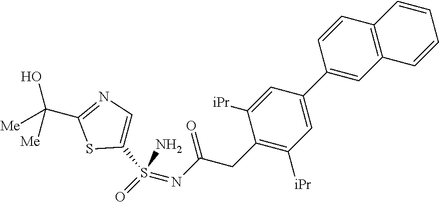 [SMILES: CC(C)c1cc(-c2ccc3ccccc3c2)cc(C(C)C)c1CC(=O)N=[S@](N)(=O)c1cnc(C(C)(C)O)s1] |
| 249b | 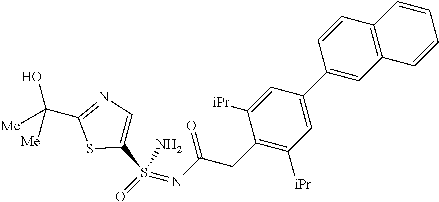 [SMILES: CC(C)c1cc(-c2ccc3ccccc3c2)cc(C(C)C)c1CC(=O)N=[S@@](N)(=O)c1cnc(C(C)(C)O)s1] |
| 250 | 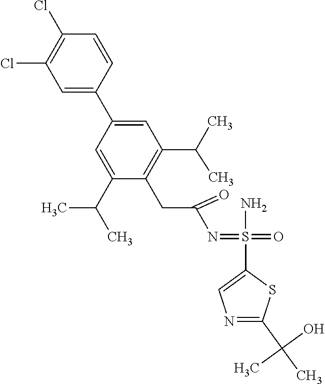 [SMILES: CC(C)c1cc(-c2ccc(Cl)c(Cl)c2)cc(C(C)C)c1CC(=O)N=S(N)(=O)c1cnc(C(C)(C)O)s1] |
| 251 | 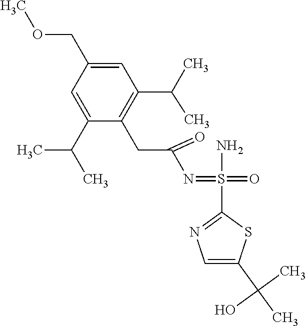 [SMILES: COCc1cc(C(C)C)c(CC(=O)N=S(N)(=O)c2ncc(C(C)(C)O)s2)c(C(C)C)c1] |
| 251a | 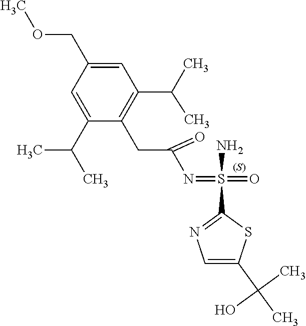 |
| 251b | 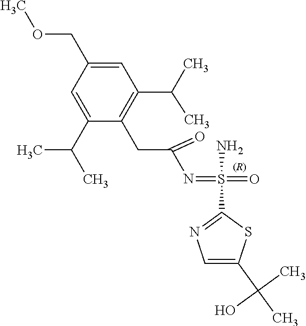 |
| 252 | 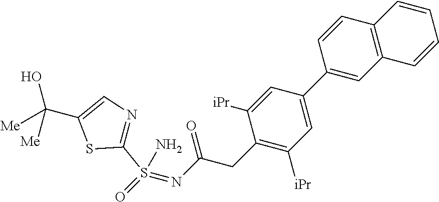 [SMILES: CC(C)c1cc(-c2ccc3ccccc3c2)cc(C(C)C)c1CC(=O)N=S(N)(=O)c1ncc(C(C)(C)O)s1] |
| 253 | 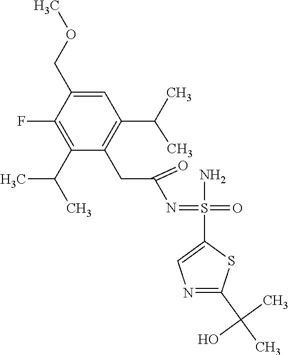 [SMILES: COCc1cc(C(C)C)c(CC(=O)N=S(N)(=O)c2cnc(C(C)(C)O)s2)c(C(C)C)c1F] |
| 254 | 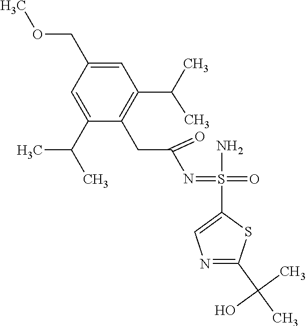 [SMILES: COCc1cc(C(C)C)c(CC(=O)N=S(N)(=O)c2cnc(C(C)(C)O)s2)c(C(C)C)c1] |
| 255 | 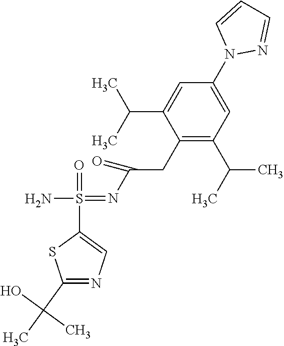 [SMILES: CC(C)c1cc(-n2cccn2)cc(C(C)C)c1CC(=O)N=S(N)(=O)c1cnc(C(C)(C)O)s1] |
| 256 | 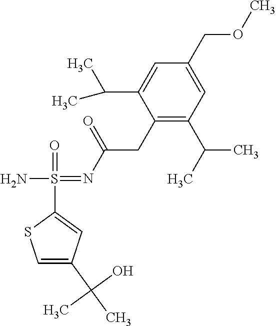 [SMILES: COCc1cc(C(C)C)c(CC(=O)N=S(N)(=O)c2cc(C(C)(C)O)cs2)c(C(C)C)c1] |
| 256a | 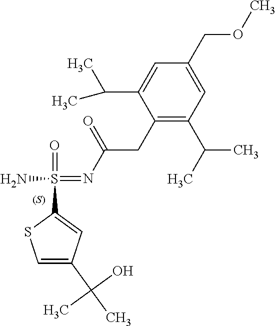 |
| 256b | 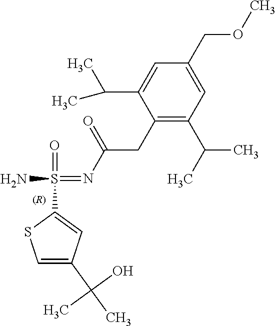 |
| 257 | 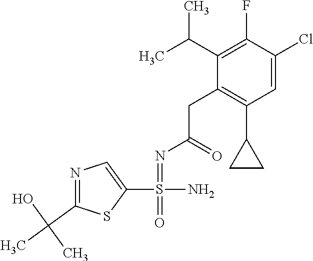 [SMILES: CC(C)c1c(F)c(Cl)cc(C2CC2)c1CC(=O)N=S(N)(=O)c1cnc(C(C)(C)O)s1] |
| 258 | 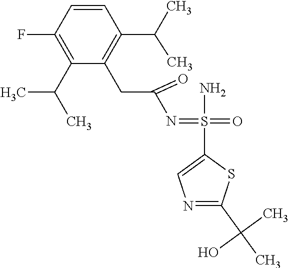 [SMILES: CC(C)c1ccc(F)c(C(C)C)c1CC(=O)N=S(N)(=O)c1cnc(C(C)(C)O)s1] |
| 258a | 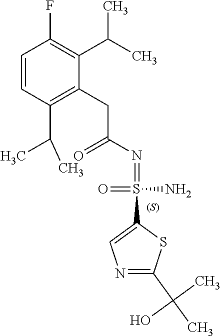 |
| 258b | 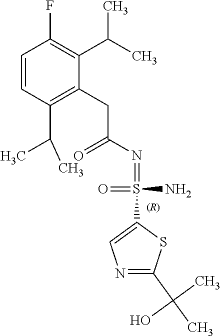 |
| 259 | 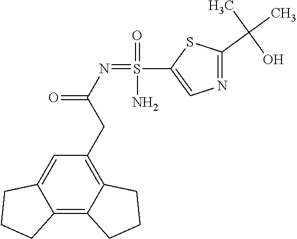 [SMILES: CC(C)(O)c1ncc(S(N)(=O)=NC(=O)Cc2cc3c(c4c2CCC4)CCC3)s1] |
| 259a | 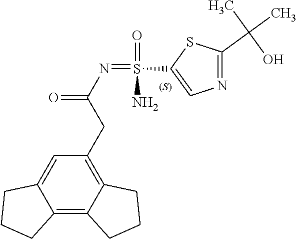 |
| 259b | 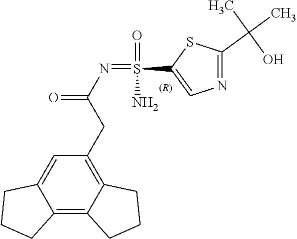 |
| 260 | 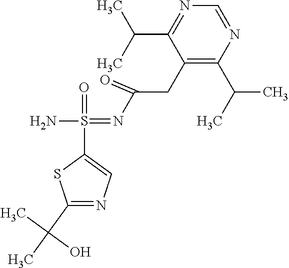 [SMILES: CC(C)c1ncnc(C(C)C)c1CC(=O)N=S(N)(=O)c1cnc(C(C)(C)O)s1] |
| 261a | 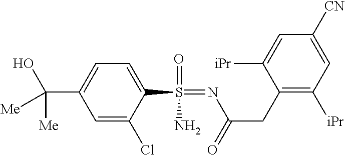 [SMILES: CC(C)c1cc(C#N)cc(C(C)C)c1CC(=O)N=[S@](N)(=O)c1ccc(C(C)(C)O)cc1Cl] |
| 261b | 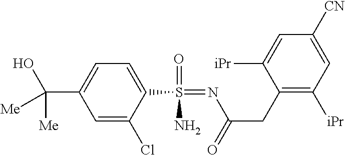 [SMILES: CC(C)c1cc(C#N)cc(C(C)C)c1CC(=O)N=[S@@](N)(=O)c1ccc(C(C)(C)O)cc1Cl] |
| 262a | 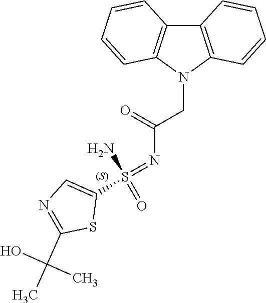 |
| 262b | 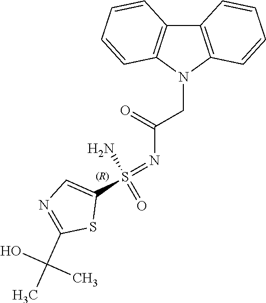 |
| 263 | 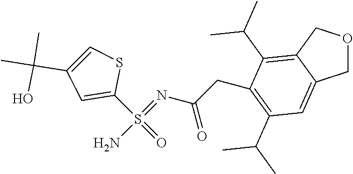 [SMILES: CC(C)c1cc2c(c(C(C)C)c1CC(=O)N=S(N)(=O)c1cc(C(C)(C)O)cs1)COC2] |
| 263a | 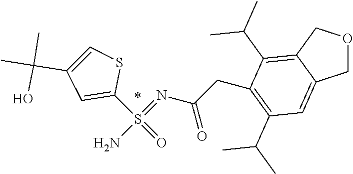 [SMILES: CC(C)c1cc2c(c(C(C)C)c1CC(=O)N=S(N)(=O)c1cc(C(C)(C)O)cs1)COC2] |
| 263b | 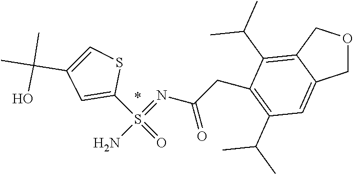 [SMILES: CC(C)c1cc2c(c(C(C)C)c1CC(=O)N=S(N)(=O)c1cc(C(C)(C)O)cs1)COC2] |
| 264 | 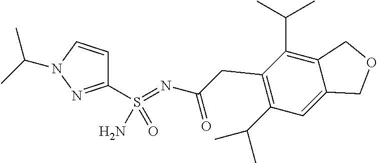 [SMILES: CC(C)c1cc2c(c(C(C)C)c1CC(=O)N=S(N)(=O)c1ccn(C(C)C)n1)COC2] |
| 264a | 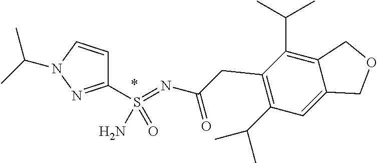 [SMILES: CC(C)c1cc2c(c(C(C)C)c1CC(=O)N=S(N)(=O)c1ccn(C(C)C)n1)COC2] |
| 264b | 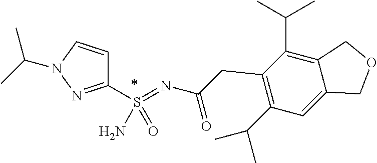 [SMILES: CC(C)c1cc2c(c(C(C)C)c1CC(=O)N=S(N)(=O)c1ccn(C(C)C)n1)COC2] |

and pharmaceutically acceptable salts thereof.

In some embodiments, provided herein is a compound that is selected from the group consisting of the compounds in Table 1-3:

TABLE 1-3

| | |
|---|---|
| 301 | 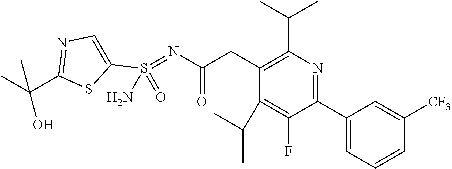 [SMILES: CC(C)c1nc(-c2cccc(C(F)(F)F)c2)c(F)c(C(C)C)c1CC(=O)N=S(N)(=O)c1cnc(C(C)(C)O)s1] |
| 302 | 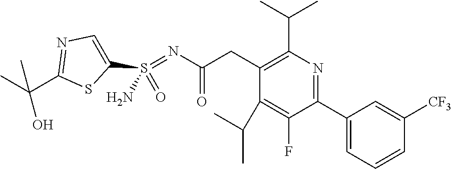 [SMILES: CC(C)c1nc(-c2cccc(C(F)(F)F)c2)c(F)c(C(C)C)c1CC(=O)N=[S@@](N)(=O)c1cnc(C(C)(C)O)s1] |
| 303 | 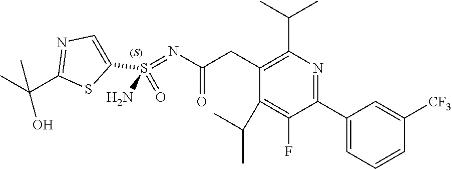 |
| 304 | 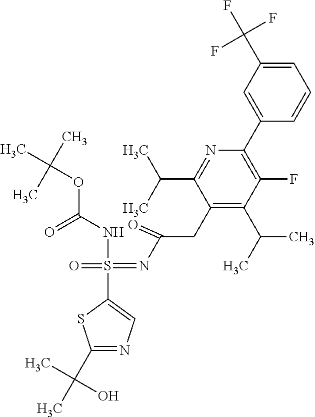 [SMILES: CC(C)c1nc(-c2cccc(C(F)(F)F)c2)c(F)c(C(C)C)c1CC(=O)N=S(=O)(NC(=O)OC(C)(C)C)c1cnc(C(C)(C)O)s1] |
| 305 | 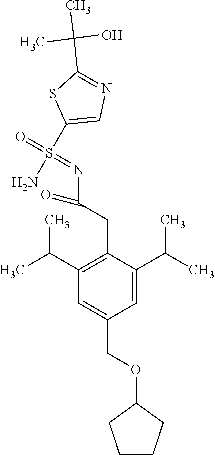 [SMILES: CC(C)c1cc(COC2CCCC2)cc(C(C)C)c1CC(=O)N=S(N)(=O)c1cnc(C(C)(C)O)s1] |
| 306 | 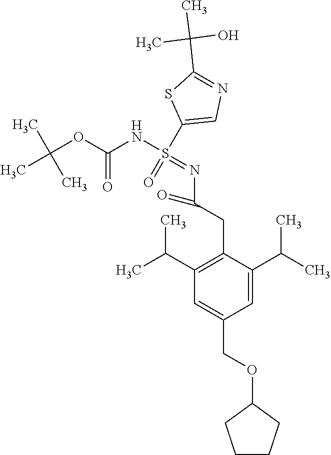 [SMILES: CC(C)c1cc(COC2CCCC2)cc(C(C)C)c1CC(=O)N=S(=O)(NC(=O)OC(C)(C)C)c1cnc(C(C)(C)O)s1] |
| 307 | 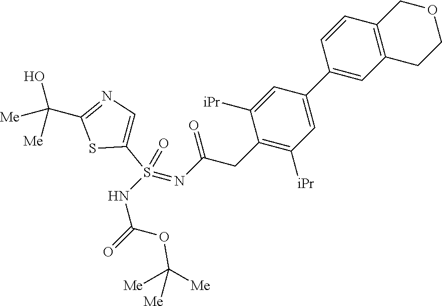 [SMILES: CC(C)c1cc(-c2ccc3c(c2)CCOC3)cc(C(C)C)c1CC(=O)N=S(=O)(NC(=O)OC(C)(C)C)c1cnc(C(C)(C)O)s1] |
| 309 | 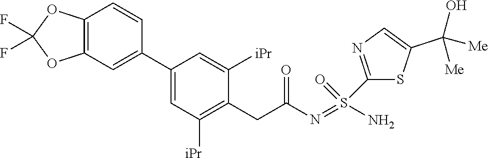 [SMILES: CC(C)c1cc(-c2ccc3c(c2)OC(F)(F)O3)cc(C(C)C)c1CC(=O)N=S(N)(=O)c1ncc(C(C)(C)O)s1] |
| 309a | 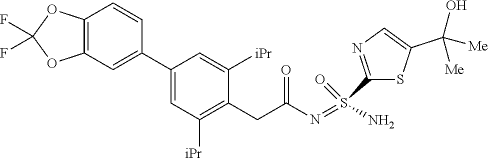 [SMILES: CC(C)c1cc(-c2ccc3c(c2)OC(F)(F)O3)cc(C(C)C)c1CC(=O)N=[S@@](N)(=O)c1ncc(C(C)(C)O)s1] |
| 309b | 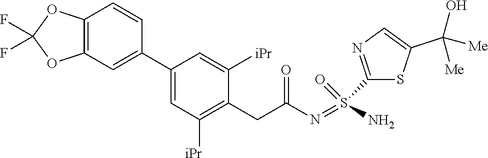 [SMILES: CC(C)c1cc(-c2ccc3c(c2)OC(F)(F)O3)cc(C(C)C)c1CC(=O)N=[S@](N)(=O)c1ncc(C(C)(C)O)s1] |
| 310 | 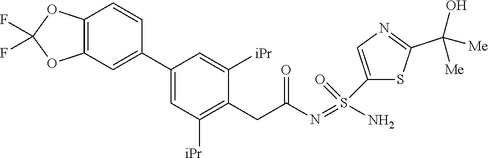 [SMILES: CC(C)c1cc(-c2ccc3c(c2)OC(F)(F)O3)cc(C(C)C)c1CC(=O)N=S(N)(=O)c1cnc(C(C)(C)O)s1] |

or a pharmaceutically acceptable salt thereof.

### Pharmaceutical Compositions and Administration

### General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, μ, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100/6 with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fir Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants. Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as crosslinked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two. e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which. (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 6 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of Formula AA | 40 weight percent to about 80 weight |
| | percent (e.g., from about 50 weight |
| | percent to about 70 weight percent, from |
| | about 55 weight percent to about 70 |
| | weight percent; from about 60 weight |
| | percent to about 65 weight percent; e.g., |
| | about 62.1 weight percent) |
| Crospovidone (Kollidon CL) | 0.5 weight percent to about 5 weight |
| | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 |
| (Pharmatose 200M) | weight percent (e.g., from about 20 |
| | weight percent to about 40 weight percent, |
| | from about 25 weight percent to about 35 |
| | weight percent; e.g., about 31.03 weight |
| | percent |
| Povidone (Kollidon K30) | about 0.5 weight percent to about 5 weight |
| | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients and amounts as shown in Table B.

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate | About 31.03 weight percent |
| (Pharmatose 200M) | |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table C.

TABLE C

| Ingredient | Weight Percent |
|---|---|
| methyl cellulose (Methocel | 0.05 weight percent to about 5 weight |
| A15C premium) | percent (e.g., from about 0.05 weight |
| | percent to about 3 weight percent, from |
| | about 0.1 weight percent to about 3 |
| | weight percent; e.g., about 1.4 weight |
| | percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 |
| | weight percent; e.g., about 1.0 weight |
| | percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight |
| | percent; e.g., about 0.20 weight percent) |
| disodium phosphate | about 0.05 weight percent to about 1 |
| dodecahydrate | weight percent (e.g., from about 0.05 |
| | weight percent to about 0.5 weight |
| | percent; e.g., about 0.15 weight percent) |
| sodium dihydrogen phospahate | about 0.005 weight percent to about 0.5 |
| dihydrate | weight percent (e.g., from about 0.005 |
| | weight percent to about 0.3 weight |
| | percent; e.g., about 0.15 weight percent) |

In certain embodiments, component (ii) includes water (up to 100%) and the ingredients and amounts as shown in Table D.

TABLE D

| Ingredient | Weight Percent |
|---|---|
| methyl cellulose (Methocel | about 1.4 weight percent |
| A15C premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate | about 0.15 weight percent |
| dodecahydrate | |
| sodium dihydrogen phospahate | about 0.15 weight percent |
| dihydrate | |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 3A-3C, which includes a plastic bottle, a breakable capsule, and a rectal cannula and single flow pack.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL: from about 0.01 mg/mL to about 5 mg/mL: from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

### Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as cardiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

### Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibit reduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (JNK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), JNK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kUbody, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)—IgG, an IgG(L)-V, a V(L)-lgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian. J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab 5 (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, N1-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻¹⁰ M, less than 1×10⁻¹⁰ M, less than 0.5×10⁻¹⁰ M, less than 1×10⁻¹¹ M, less than 0.5×10⁻¹¹ M, or less than 1×10⁻¹²M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁶ M, about 0.5×10⁻⁸ M, about 1×10⁻⁹ M, about 0.5×10⁻⁹ M, about 1×10⁻¹⁰ M, or about 0.5×10⁻⁵ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁶ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10.8 M, or about 1×10⁻⁹ M (inclusive); about 1×10⁻⁹ M to about 1×10⁻⁶ M, about 0.5×10⁻⁵ M, about 1×10⁻⁶ M, about 0.5×10⁻⁶ M, about 1×10⁻⁷ M, about 0.5×10⁻⁶ M, about 1×10⁻⁸ M, or about 0.5×10⁻⁸ M (inclusive); about 0.5×10⁻⁸ M to about 1×10⁻⁶ M, about 0.5×10⁻⁶ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁶ M, about 1×10⁻⁶ M, about 0.5×10⁻⁶ M, about 1×10⁻⁶ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁶ M, about 0.5×10⁻⁴ M, about 1×10⁻⁶ M, or about 0.5×10⁻⁶ M (inclusive); about 0.5×10⁻⁶ M to about 1×10⁻⁵ M, about 0.5×10⁻⁶ M, or about 1×10⁻⁶ M (inclusive); about 1×10⁻⁶ M to about 1×10⁻⁶ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹ (inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, about 1×10³ M⁻¹s⁻¹, or about 0.5×10⁶ M⁻¹s⁻¹ (inclusive); about 0.5×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁶ M⁻¹s⁻¹, about 1×10⁴ M's, about 0.5×10⁴ M⁻¹s⁻¹, or about 1×10 M⁻¹s⁻¹ (inclusive); about 1×10⁶ M⁻¹s⁻¹ to about 1×10⁶ M-'s⁻¹, about 0.5×10⁶ M⁻¹s⁻¹, about 1×10⁶ M⁻¹s⁻¹, about 0.5×10⁶ M⁻¹s⁻¹, about 1×10⁴ M⁻¹s⁻¹, or about 0.5×10⁴ M⁻¹s⁻¹ (inclusive); about 0.5×10⁴ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, or about 1×10⁴ M⁻¹s⁻¹ (inclusive); about 1×10⁴ M⁻¹s⁻¹ to about 1×10⁶ M⁻¹s⁻¹, about 0.5×10⁶ M⁻¹s⁻¹, about 1×10⁵ M⁻¹s⁻¹, or about 0.5×10⁵ M⁻¹s⁻¹ (inclusive); about 0.5×10⁵ M⁻¹s⁻¹ to about 1×10⁶ M⁻¹s⁻¹, about 0.5×10⁶ M⁻¹s⁻¹, or about 1×10⁵ M⁻¹s⁻¹ (inclusive); about 1×10 M⁻¹s⁻¹ to about 1×10⁶ M⁻¹s⁻¹, or about 0.5×10⁶ M⁻¹s⁻¹ (inclusive); or about 0.5×10⁶ M⁻¹s⁻¹ to about 1×10⁶ M⁻¹s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., complementary to all or a part of any one of SEQ ID NOs: 1-37).

Human TNFα CDS
(SEQ ID NO: 1)
ATGAGCACTGAAAGCATGATCCGGGACGTGGAGCTGGCCGAGGAGGCGCTCCCCAA
GAAGACAGGGGGGCCCCAGGGCTCCAGGCGGTGCTTGTTCCTCAGCCTCTTCTCCTT
CCTGATCGTGGCAGGCGCCACCACGCTCTTCTGCCTGCTGCACTTTGGAGTGATCGG
CCCCCAGAGGGAAGAGTTCCCCAGGGACCTCTCTCTAATCAGCCCTCTGGCCCAGGC
AGTCAGATCATCTTCTCGAACCCCGAGTGACAAGCCTGTAGCCCATGTTGTAGCAAA
CCCTCAAGCTGAGGGGCAGCTCCAGTGGCTGAACCGCCGGGCCAATGCCCTCCTGG
CCAATGGCGTGGAGCTGAGAGATAACCAGCTGGTGGTGCCATCAGAGGGCCTGTAC
CTCATCTACTCCCAGGTCCTCTTCAAGGGCCAAGGCTGCCCCTCCACCCATGTGCTC
CTCACCCACACCATCAGCCGCATCGCCGTCTCCTACCAGACCAAGGTCAACCTCCTC
TCTGCCATCAAGAGCCCCTGCCAGAGGGAGACCCCAGAGGGGGCTGAGGCCAAGCC
CTGGTATGAGCCCATCTATCTGGGAGGGGTCTTCCAGCTGGAGAAGGGTGACCGACT
CAGCGCTGAGATCAATCGGCCCGACTATCTCGACTTTGCCGAGTCTGGGCAGGTCTA
CTTTGGGATCATTGCCCTGTGA
Human TNFR1 CDS
(SEQ ID NO: 2)
ATGGGCCTCTCCACCGTGCCTGACCTGCTGCTGCCACTGGTGCTCCTGGAGCTGTTG
GTGGGAATATACCCCTCAGGGGTTATTGGACTGGTCCCTCACCTAGGGGACAGGGA
GAAGAGAGATAGTGTGTGTCCCCAAGGAAAATATATCCACCCTCAAAATAATTCGA
TTTGCTGTACCAAGTGCCACAAAGGAACCTACTTGTACAATGACTGTCCAGGCCCGG
GGCAGGATACGGACTGCAGGGAGTGTGAGAGCGGCTCCTTCACCGCTTCAGAAAAC
CACCTCAGACACTGCCTCAGCTGCTCCAAATGCCGAAAGGAAATGGGTCAGGTGGA
GATCTCTTCTTGCACAGTGGACCGGGACACCGTGTGTGGCTGCAGGAAGAACCAGT
ACCGGCATTATTGGAGTGAAAACCTTTTCCAGTGCTTCAATTGCAGCCTCTGCCTCA
ATGGGACCGTGCACCTCTCCTGCCAGGAGAAACAGAACACCGTGTGCACCTGCCAT
GCAGGTTTCTTTCTAAGAGAAAACGAGTGTGTCTCCTGTAGTAACTGTAAGAAAAGC
CTGGAGTGCACGAAGTTGTGCCTACCCCAGATTGAGAATGTTAAGGGCACTGAGGA
CTCAGGCACCACAGTGCTGTTGCCCCTGGTCATTTTCTTTGGTCTTTGCCTTTTATCCC
TCCTCTTCATTGGTTTAATGTATCGCTACCAACGGTGGAAGTCCAAGCTCTACTCCAT
TGTTTGTGGGAAATCGACACCTGAAAAAGAGGGGGAGCTTGAAGGAACTACTACTA
AGCCCCTGGCCCCAAACCCAAGCTTCAGTCCCACTCCAGGCTTCACCCCCACCCTGG
GCTTCAGTCCCGTGCCCAGTTCCACCTTCACCTCCAGCTCCACCTATACCCCCGGTGA
CTGTCCCAACTTTGCGGCTCCCCGCAGAGAGGTGGCACCACCCTATCAGGGGGCTGA
CCCCATCCTTGCGACAGCCCTCGCCTCCGACCCCATCCCCAACCCCCTTCAGAAGTG
GGAGGACAGCGCCCACAAGCCACAGAGCCTAGACACTGATGACCCCGCGACGCTGT
ACGCCGTGGTGGAGAACGTGCCCCCGTTGCGCTGGAAGGAATTCGTGCGGCGCCTA
GGGCTGAGCGACCACGAGATCGATCGGCTGGAGCTGCAGAACGGGCGCTGCCTGCG
CGAGGCGCAATACAGCATGCTGGCGACCTGGAGGCGGCGCACGCCGCGGCGCGAG
GCCACGCTGGAGCTGCTGGGACGCGTGCTCCGCGACATGGACCTGCTGGGCTGCCT
GGAGGACATCGAGGAGGCGCTTTGCGGCCCCGCCGCCCTCCCGCCCGCGCCCAGTC
TTCTCAGATGA
Human TNFR2 CDS
(SEQ ID NO: 3)
ATTCTTCCCCTGGTGGCCATGGGACCCAGGTCAATGTCACCTGCATCGTGAACGTCT
GTAGCAGCTCTGACCACAGCTCACAGTGCTCCTCCCAAGCCAGCTCCACAATGGGA
GACACAGATTCCAGCCCCTCGGAGTCCCCGAAGGACGAGCAGGTCCCCTTCTCCAA
GGAGGAATGTGCCTTTCGGTCACAGCTGGAGACGCCAGAGACCCTGCTGGGGAGCA
CCGAAGAGAAGCCCCTGCCCCTTGGAGTGCCTGATGCTGGGATGAAGCCCAGTTAA
Human TRADD CDS
(SEQ ID NO: 4)
ATGGCAGCTGGGCAAAATGGGCACGAAGAGTGGGTGGGCAGCGCATACCTGTTTGT
GGAGTCCTCGCTGGACAAGGTGGTCCTGTCGGATGCCTACGCGCACCCCCAGCAGA
AGGTGGCAGTGTACAGGGCTCTGCAGGCTGCCTTGGCAGAGAGCGGCGGGAGCCCG
GACGTGCTGCAGATGCTGAAGATCCACCGCAGCGACCCGCAGCTGATCGTGCAGCT
GCGATTCTGCGGGCGGCAGCCCTGTGGCCGCTTCCTCCGCGCCTACCGCGAGGGGGC
GCTGCGCGCCGCGCTGCAGAGGAGCCTGGCGGCCGCGCTCGCCCAGCACTCGGTGC
CGCTGCAACTGGAGCTGCGCGCCGGCGCCGAGCGGCTGGACGCTTTGCTGGCGGAC
GAGGAGCGCTGTTTGAGTTGCATCCTAGCCCAGCAGCCCGACCGGCTCCGGGATGA
AGAACTGGCTGAGCTGGAGGATGCGCTGCGAAATCTGAAGTGCGGCTCGGGGGCCC
GGGGTGGCGACGGGGAGGTCGCTTCGGCCCCCTTGCAGCCCCCGGTGCCCTCTCTGT
CGGAGGTGAAGCCGCCGCCGCCGCCGCCACCTGCCCAGACTTTTCTGTTCCAGGGTC
0AGCCTGTAGTGAATCGGCCGCTGAGCCTGAAGGACCAACAGACGTTCGCGCGCTCT
GTGGGTCTCAAATGGCGCAAGGTGGGGCGCTCACTGCAGCGAGGCTGCCGGGCGCT
GCGGGACCCGGCGCTGGACTCGCTGGCCTACGAGTACGAGCGCGAGGGACTGTACG
AGCAGGCCTTCCAGCTGCTGCGGCGCTTCGTGCAGGCCGAGGGCCGCCGCGCCACG
CTGCAGCGCCTGGTGGAGGCACTCGAGGAGAACGAGCTCACCAGCCTGGCAGAGGA
CTTGCTGGGCCTGACCGATCCCAATGGCGGCCTGGCCTAG
Human TRAF2 CDS
(SEQ ID NO: 5)
ATGGCTGCAGCTAGCGTGACCCCCCCTGGCTCCCTGGAGTTGCTACAGCCCGGCTTC
TCCAAGACCCTCCTGGGGACCAAGCTGGAAGCCAAGTACCTGTGCTCCGCCTGCAG
AAACGTCCTCCGCAGGCCCTTCCAGGCGCAGTGTGGCCACCGGTACTGCTCCTTCTG
CCTGGCCAGCATCCTCAGCTCTGGGCCTCAGAACTGTGCTGCCTGTGTTCACGAGGG
CATATATGAAGAAGGCATTTCTATTTTAGAAAGCAGTTCGGCCTTCCCAGATAATGC
TGCCCGCAGGGAGGTGGAGAGCCTGCCGGCCGTCTGTCCCAGTGATGGATGCACCT
GGAAGGGGACCCTGAAAGAATACGAGAGCTGCCACGAAGGCCGCTGCCCGCTCATG
CTGACCGAATGTCCCGCGTGCAAAGGCCTGGTCCGCCTTGGTGAAAAGGAGCGCCA
CCTGGAGCACGAGTGCCCGGAGAGAAGCCTGAGCTGCCGGCATTGCCGGGCACCCT
GCTGCGGAGCAGACGTGAAGGCGCACCACGAGGTCTGCCCCAAGTTCCCCTTAACT
TGTGACGGCTGCGGCAAGAAGAAGATCCCCCGGGAGAAGTTTCAGGACCACGTCAA
GACTTGTGGCAAGTGTCGAGTCCCTTGCAGATTCCACGCCATCGGCTGCCTCGAGAC
GGTAGAGGGTGAGAAACAGCAGGAGCACGAGGTGCAGTGGCTGCGGGAGCACCTG
GCCATGCTACTGAGCTCGGTGCTGGAGGCAAAGCCCCTCTTGGGAGACCAGAGCCA
CGCGGGGTCAGAGCTCCTGCAGAGGTGCGAGAGCCTGGAGAAGAAGACGGCCACTT
TTGAGAACATTGTCTGCGTCCTGAACCGGGAGGTGGAGAGGGTGGCCATGACTGCC
GAGGCCTGCAGCCGGCAGCACCGGCTGGACCAAGACAAGATTGAAGCCCTGAGTAG
CAAGGTGCAGCAGCTGGAGAGGAGCATTGGCCTCAAGGACCTGGCGATGGCTGACT
TGGAGCAGAAGGTCTTGGAGATGGAGGCATCCACCTACGATGGGGTCTTCATCTGG
AAGATCTCAGACTTCGCCAGGAAGCGCCAGGAAGCTGTGGCTGGCCGCATACCCGC
CATCTTCTCCCCAGCCTTCTACACCAGCAGGTACGGCTACAAGATGTGTCTGCGTAT
CTACCTGAACGGCGACGGCACCGGGCGAGGAACACACCTGTCCCTCTTCTTTGTGGT
GATGAAGGGCCCGAATGACGCCCTGCTGCGGTGGCCCTTCAACCAGAAGGTGACCT
TAATGCTGCTCGACCAGAATAACCGGGAGCACGTGATTGACGCCTTCAGGCCCGAC
GTGACTTCATCCTCTTTTCAGAGGCCAGTCAACGACATGAACATCGCAAGCGGCTGC
CCCCTCTTCTGCCCCGTCTCCAAGATGGAGGCAAAGAATTCCTACGTGCGGGACGAT
GCCATCTTCATCAAGGCCATTGTGGACCTGACAGGGCTCTAA
Human AP-1 CDS
(SEQ ID NO: 6)
ATGGAAACACCCTTCTACGGCGATGAGGCGCTGAGCGGCCTGGGCGGCGGCGCCAG
TGGCAGCGGCGGCAGCTTCGCGTCCCCGGGCCGCTTGTTCCCCGGGGCGCCCCCGAC
GGCCGCGGCCGGCAGCATGATGAAGAAGGACGCGCTGACGCTGAGCCTGAGTGAGC
AGGTGGCGGCAGCGCTCAAGCCTGCGGCCGCGCCGCCTCCTACCCCCCTGCGCGCC
GACGGCGCCCCCAGCGCGGCACCCCCCGACGGCCTGCTCGCCTCTCCCGACCTGGG
GCTGCTGAAGCTGGCCTCCCCCGAGCTCGAGCGCCTCATCATCCAGTCCAACGGGCT
GGTCACCACCACGCCGACGAGCTCACAGTTCCTCTACCCCAAGGTGGCGGCCAGCG
AGGAGCAGGAGTTCGCCGAGGGCTTCGTCAAGGCCCTGGAGGATTTACACAAGCAG
AACCAGCTCGGCGCGGGCGCGGCCGCTGCCGCCGCCGCCGCCGCCGCCGGGGGGCC
CTCGGGCACGGCCACGGGCTCCGCGCCCCCCGGCGAGCTGGCCCCGGCGGCGGCCG
CGCCCGAAGCGCCTGTCTACGCGAACCTGAGCAGCTACGCGGGCGGCGCCGGGGGC
GCGGGGGGCGCCGCGACGGTCGCCTTCGCTGCCGAACCTGTGCCCTTCCCGCCGCCG
CCACCCCCAGGCGCGTTGGGGCCGCCGCGCCTGGCTGCGCTCAAGGACGAGCCACA
GACGGTGCCCGACGTGCCGAGCTTCGGCGAGAGCCCGCCGTTGTCGC
CCATCGACATGGACACGCAGGAGCGCATCAAGGCGGAGCGCAAGCGGCTGCGCAA
CCGCATCGCCGCCTCCAAGTGCCGCAAGCGCAAGCTGGAGCGCATCTCGCGCCTGG
AAGAGAAAGTGAAGACCCTCAAGAGTCAGAACACGGAGCTGGCGTCCACGGCGAG
CCTGCTGCGCGAGCAGGTGGCGCAGCTCAAGCAGAAAGTCCTCAGCCACGTCAACA
GCGGCTGCCAGCTGCTGCCCCAGCACCAGGTGCCCGCGTACTGA
Human ASK1 CDS
(SEQ ID NO: 7)
ATGAGCACGGAGGCGGACGAGGGCATCACTTTCTCTGTGCCACCCTTCGCCCCCTCG
GGCTTCTGCACCATCCCCGAGGGCGGCATCTGCAGGAGGGGAGGAGCGGCGGCGGT
GGGCGAGGGCGAGGAGCACCAGCTGCCACCGCCGCCGCCGGGCAGTTTCTGGAACG
TGGAGAGCGCCGCTGCCCCTGGCATCGGTTGTCCGGCGGCCACCTCCTCGAGCAGTG
CCACCCGAGGCCGGGGCAGCTCTGTTGGCGGGGGCAGCCGACGGACCACGGTGGCA
TATGTGATCAACGAAGCGAGCCAAGGGCAACTGGTGGTGGCCGAGAGCGAGGCCCT
GCAGAGCTTGCGGGAGGCGTGCGAGACAGTGGGCGCCACCCTGGAACCCTGCATTT
TGGGAAACTCGACTTTGGAGAAACCACCGTGCTGGACCGCTTTTACAATGCAGATAT
TGCGGTGGTGGAGATGAGCGATGCCTTCCGGCAGCCGTCCTTGTTTTACCACCTTGG
GGTGAGAGAAAGTTTCAGCATGGCCAACAACATCATCCTCTACTGCGATACTAACTC
GGACTCTCTGCAGTCACTGAAGGAAATCATTTGCCAGAAGAATACTATGTGCACTGG
GAACTACACCTTTGTTCCTTACATGATAACTCCACATAACAAAGTCTACTGCTGTGA
CAGCAGCTTCATGAAGGGGTTGACAGAGCTCATGCAACCGAACTTCGAGCTGCTTCT
TGGACCCATCTGCTTACCTCTTGTGGATCGTTTTATTCAACTTTTGAAGGTGGCACAA
GCAAGTTCTAGCCAGTACTTCCGGGAATCTATACTCAATGACATCAGGAAAGCTCGT
AATTTATACACTGGTAAAGAATTGGCAGCTGAGTTGGCAAGAATTCGGCAGCGAGT
AGATAATATCGAAGTCTTGACAGCAGATATTGTCATAAATCTGTTACTTTCCTACAG
AGATATCCAGGACTATGATTCTATTGTGAAGCTGGTAGAGACTTTAGAAAAACTGCC
AACCTTTGATTTGGCCTCCCATCACCATGTGAAGTTTCATTATGCATTTGCACTGAAT
AGGAGAAATCTCCCTGGTGACAGAGCAAAAGCTCTTGATATTATGATTCCCATGGTG
CAAAGCGAAGGACAAGTTGCTTCAGATATGTATTGCCTAGTTGGTCGAATCTACAAA
GATATGTTTTTGGACTCTAATTTCACGGACACTGAAAGCAGAGACCATGGAGCTTCT
TGGTTCAAAAAGGCATTTGAATCTGAGCCAACACTACAGTCAGGAATTAATTATGCG
GTCCTCCTCCTGGCAGCTGGACACCAGTTTGAATCTTCCTTTGAGCTCCGGAAAGTT
GGGGTGAAGCTAAGTAGTCTTCTTGGTAAAAAGGGAAACTTGGAAAAACTCCAGAG
CTACTGGGAAGTTGGATTTTTTCTGGGGGCCAGCGTCCTAGCCAATGACCACATGAG
AGTCATTCAAGCATCTGAAAAGCTTTTTAAACTGAAGACACCAGCATGGTACCTCAA
GTCTATTGTAGAGACAATTTTGATATATAAGCATTTTGTGAAACTGACCACAGAACA
GCCTGTGGCCAAGCAAGAACTTGTGGACTTTTGGATGGATTTCCTGGTCGAGGCCAC
AAAGACAGATGTTACTGTGGTTAGGTTTCCAGTATTAATATTAGAACCAACCAAAAT
CTATCAACCTTCTTATTTGTCTATCAACAATGAAGTTGAGGAAAAGACAATCTCTAT
TTGGCACGTGCTTCCTGATGACAAGAAAGGTATACATGAGTGGAATTTTAGTGCCTC
TTCTGTCAGGGGAGTGAGTATTTCTAAATTTGAAGAAAGATGCTGCTTTCTTTATGTG
CTTCACAATTCTGATGATTTCCAAATCTATTTCTGTACAGAACTTCATTGTAAAAAGT
TTTTTGAGATGGTGAACACCATTACCGAAGAGAAGGGGAGAAGCACAGAGGAAGG
AGACTGTGAAAGTGACTTGCTGGAGTATGACTATGAATATGATGAAAATGGTGACA
GAGTCGTTTTAGGAAAAGGCACTTATGGGATAGTCTACGCAGGTCGGGACTTGAGC
AACCAAGTCAGAATTGCTATTAAGGAAATCCCAGAGAGAGACAGCAGATACTCTCA
GCCCCTGCATGAAGAAATAGCATTGCATAAACACCTGAAGCACAAAAATATTGTCC
AGTATCTGGGCTCTTTCAGTGAGAATGGTTTCATTAAAATCTTCATGGAGCAGGTCC
CTGGAGGAAGTCTTTCTGCTCTCCTTCGTTCCAAATGGGGTCCATTAAAAGACAATG
AGCAAACAATTGGCTTTTATACAAAGCAAATACTGGAAGGATTAAAATATCTCCATG
ACAATCAGATAGTTCACCGGGACATAAAGGGTGACAATGTGTTGATTAATACCTAC
AGTGGTGTTCTCAAGATCTCTGACTTCGGAACATCAAAGAGGCTTGCTGGCATAAAC
CCCTGTACTGAAACTTTTACTGGTACCCTCCAGTATATGGCACCAGAAATAATAGAT
AAAGGACCAAGAGGCTACGGAAAAGCAGCAGACATCTGGTCTCTGGGCTGTACAAT
CATTGAAATGGCCACAGGAAAACCCCCATTTTATGAACTGGGAGAACCACAAGCAG
CTATGTTCAAGGTGGGAATGTTTAAAGTCCACCCTGAGATCCCAGAGTCCATGTCTG
CAGAGGCCAAGGCATTCATACTGAAATGTTTTGAACCAGATCCTGACAAGAGAGCC
TGTGCTAACGACTTGCTTGTTGATGAGTTTTTAAAAGTTTCAAGCAAAAAGAAAAAG
ACACAACCTAAGCTTTCAGCTCTTTCAGCTGGATCAAATGAATATCTCAGGAGTATA
TCCTTGCCGGTACCTGTGCTGGTGGAGGACACCAGCAGCAGCAGTGAGTACGGCTC
AGTTTCACCCGACACGGAGTTGAAAGTGGACCCCTTCTCTTTCAAAACAAGAGCCAA
GTCCTGCGGAGAAAGAGATGTCAAGGGAATTCGGACACTCTTTTTGGGCATTCCAGA
TGAGAATTTTGAAGATCACAGTGCTCCTCCTTCCCCTGAAGAAAAAGATTCTGGATT
CTTCATGCTGAGGAAGGACAGTGAGAGGCGAGCTACCCTTCACAGGATCCTGACGG
AAGACCAAGACAAAATTGTGAGAAACCTAATGGAATCTTTAGCTCAGGGGGCTGAA
GAACCGAAACTAAAATGGGAACACATCACAACCCTCATTGCAAGCCTCAGAGAATT
TGTGAGATCCACTGACCGAAAAATCATAGCCACCACACTGTCAAAGCTGAAACTGG
AGCTGGACTTCGACAGCCATGGCATTAGCCAAGTCCAGGTGGTACTCTTTGGTTTTC
AAGATGCTGTCAATAAAGTTCTTCGGAATCATAACATCAAGCCGCACTGGATGTTTG
CCTTAGACAGTATCATTCGGAAGGCGGTACAGACAGCCATTACCATCCTGGTTCCAG
AACTAAGGCCACATTTCAGCCTTGCATCTGAGAGTGATACTGCTGATCAAGAAGACT
TGGATGTAGAAGATGACCATGAGGAACAGCCTTCAAATCAAACTGTCCGAAGACCT
CAGGCTGTCATTGAAGATGCTGTGGCTACCTCAGGCGTGAGCACGCTCAGTTCTACT
GTGTCTCATGATTCCCAGAGTGCTCACCGGTCACTGAATGTACAGCTTGGAAGGATG
AAAATAGAAACCAATAGATTACTGGAAGAATTGGTTCGGAAAGAGAAAGAATTACA
AGCACTCCTTCATCGAGCTATTGAAGAAAAAGACCAAGAAATTAAACACCTGAAGC
TTAAGTCCCAACCCATAGAAATTCCTGAATTGCCTGTATTTCATCTAAATTCTTCTGG
CACAAATACTGAAGATTCTGAACTTACCGACTGGCTGAGAGTGAATGGAGCTGATG
AAGACACTATAAGCCGGTTTTTGGCTGAAGATTATACACTATTGGATGTTCTCTACT
ATGTTACACGTGATGACTTAAAATGCTTGAGACTAAGGGGAGGGATGCTGTGCACA
CTGTGGAAGG CTATCATTGACTTTCGAAACAAACAGACTTGA
Human CD14 CDS
(SEQ ID NO: 8)
ATGGAGCGCGCGTCCTGCTTGTTGCTGCTGCTGCTGCCGCTGGTGCACGTCTCTGCG
ACCACGCCAGAACCTTGTGAGCTGGACGATGAAGATTTCCGCTGCGTCTGCAACTTC
TCCGAACCTCAGCCCGACTGGTCCGAAGCCTTCCAGTGTGTGTCTGCAGTAGAGGTG
GAGATCCATGCCGGCGGTCTCAACCTAGAGCCGTTTCTAAAGCGCGTCGATGCGGA
CGCCGACCCGCGGCAGTATGCTGACACGGTCAAGGCTCTCCGCGTGCGGCGGCTCA
CAGTGGGAGCCGCACAGGTTCCTGCTCAGCTACTGGTAGGCGCCCTGCGTGTGCTAG
CGTACTCCCGCCTCAAGGAACTGACGCTCGAGGACCTAAAGATAACCGGCACCATG
CCTCCGCTGCCTCTGGAAGCCACAGGACTTGCACTTTCCAGCTTGCGCCTACGCAAC
GTGTCGTGGGCGACAGGGCGTTCTTGGCTCGCCGAGCTGCAGCAGTGGCTCAAGCC
AGGCCTCAAGGTACTGAGCATTGCCCAAGCACACTCGCCTGCCTTTTCCTGCGAACA
GGTTCGCGCCTTCCCGGCCCTTACCAGCCTAGACCTGTCTGACAATCCTGGACTGGG
CGAACGCGGACTGATGGCGGCTCTCTGTCCCCACAAGTTCCCGGCCATCCAGAATCT
AGCGCTGCGCAACACAGGAATGGAGACGCCCACAGGCGTGTGCGCCGCACTGGCGG
CGGCAGGTGTGCAGCCCCACAGCCTAGACCTCAGCCACAACTCGCTGCGCGCCACC
GTAAACCCTAGCGCTCCGAGATGCATGTGGTCCAGCGCCCTGAACTCCCTCAATCTG
TCGTTCGCTGGGCTGGAACAGGTGCCTAAAGGACTGCCAGCCAAGCTCAGAGTGCT
CGATCTCAGCTGCAACAGACTGAACAGGGCGCCGCAGCCTGACGAGCTGCCCGAGG
TGGATAACCTGACACTGGACGGGAATCCCTTCCTGGTCCCTGGAACTGCCCTCCCCC
ACGAGGGCTCAATGAACTCCGGCGTGGTCCCAGCCTGTGCACGTTCGACCCTGTCGG
TGGGGGTGTCGGGAACCCTGGTGCTGCTCCAAGGGGCCCGGGGCTTTGCCTAA
Human ERK1 CDS
(SEQ ID NO: 9)
ATGGCGGCGGCGGCGGCTCAGGGGGGCGGGGGCGGGGAGCCCCGTAGAACCGAGG
GGGTCGGCCCGGGGGTCCCGGGGGAGGTGGAGATGGTGAAGGGGCAGCCGTTCGAC
GTGGGCCCGCGCTACACGCAGTTGCAGTACATCGGCGAGGGCGCGTACGGCATGGT
CAGCTCGGCCTATGACCACGTGCGCAAGACTCGCGTGGCCATCAAGAAGATCAGCC
CCTTCGAACATCAGACCTACTGCCAGCGCACGCTCCGGGAGATCCAGATCCTGCTGC
GCTTCCGCCATGAGAATGTCATCGGCATCCGAGACATTCTGCGGGCGTCCACCCTGG
AAGCCATGAGAGATGTCTACATTGTGCAGGACCTGATGGAGACTGACCTGTACAAG
TTGCTGAAAAGCCAGCAGCTGAGCAATGACCATATCTGCTACTTCCTCTACCAGATC
CTGCGGGGCCTCAAGTACATCCACTCCGCCAACGTGCTCCACCGAGATCTAAAGCCC
TCCAACCTGCTCATCAACACCACCTGCGACCTTAAGATTTGTGATTTCGGCCTGGCC
CGGATTGCCGATCCTGAGCATGACCACACCGGCTTCCTGACGGAGTATGTGGCTACG
CGCTGGTACCGGGCCCCAGAGATCATGCTGAACTCCAAGGGCTATACCAAGTCCAT
CGACATCTGGTCTGTGGGCTGCATTCTGGCTGAGATGCTCTCTAACCGGCCCATCTTC
CCTGGCAAGCACTACCTGGATCAGCTCAACCACATTCTGGGCATCCTGGGCTCCCCA
TCCCAGGAGGACCTGAATTGTATCATCAACATGAAGGCCCGAAACTACCTACAGTCT
CTGCCCTCCAAGACCAAGGTGGCTTGGGCCAAGCTTTTCCCCAAGTCAGACTCCAAA
GCCCTTGACCTGCTGGACCGGATGTTAACCTTTAACCCCAATAAACGGATCACAGTG
GAGGAAGCGCTGGCTCACCCCTACCTGGAGCAGTACTATGACCCGACGGATGAGCC
AGTGGCCGAGGAGCCCTTCACCTTCGCCATGGAGCTGGATGACCTACCTAAGGAGC
GGCTGAAGGAGCTCATCTTCCAGGAGACAGCACGCTTCCAGCCCGGAGTGCTGGAG
GCCCCCTAG
Human ERK2 CDS
(SEQ ID NO: 10)
ATGGCGGCGGCGGCGGCGGCGGGCGCGGGCCCGGAGATGGTCCGCGGGCAGGTGTT
CGACGTGGGGCCGCGCTACACCAACCTCTCGTACATCGGCGAGGGCGCCTACGGCA
TGGTGTGCTCTGCTTATGATAATGTCAACAAAGTTCGAGTAGCTATCAAGAAAATCA
GCCCCTTTGAGCACCAGACCTACTGCCAGAGAACCCTGAGGGAGATAAAAATCTTA
CTGCGCTTCAGACATGAGAACATCATTGGAATCAATGACATTATTCGAGCACCAACC
ATCGAGCAAATGAAAGATGTATATATAGTACAGGACCTCATGGAAACAGATCTTTA
CAAGCTCTTGAAGACACAACACCTCAGCAATGACCATATCTGCTATTTTCTCTACCA
GATCCTCAGAGGGTTAAAATATATCCATTCAGCTAACGTTCTGCACCGTGACCTCAA
GCCTTCCAACCTGCTGCTCAACACCACCTGTGATCTCAAGATCTGTGACTTTGGCCT
GGCCCGTGTTGCAGATCCAGACCATGATCACACAGGGTTCCTGACAGAATATGTGGC
CACACGTTGGTACAGGGCTCCAGAAATTATGTTGAATTCCAAGGGCTACACCAAGTC
CATTGATATTTGGTCTGTAGGCTGCATTCTGGCAGAAATGCTTTCTAACAGGCCCAT
CTTTCCAGGGAAGCATTATCTTGACCAGCTGAACCACATTTTGGGTATTCTTGGATCC
CCATCACAAGAAGACCTGAATTGTATAATAAATTTAAAAGCTAGGAACTATTTGCTT
TCTCTTCCACACAAAAATAAGGTGCCATGGAACAGGCTGTTCCCAAATGCTGACTCC
AAAGCTCTGGACTTATTGGACAAAATGTTGACATTCAACCCACACAAGAGGATTGA
AGTAGAACAGGCTCTGGCCCACCCATATCTGGAGCAGTATTACGACCCGAGTGACG
AGCCCATCGCCGAAGCACCATTCAAGTTCGACATGGAATTGGATGACTTGCCTAAGG
AAAAGCTCAAAGAACTAATTTTTGAAGAGACTGCTAGATTCCAGCCAGGATACAGA
TCTTAA
Human IKK CDS
(SEQ ID NO: 11)
ATGTTTTCAGGGGGGTGTCATAGCCCCGGGTTTGGCCGCCCCAGCCCCGCCTTCCCC
GCCCCGGGGAGCCCGCCCCCTGCCCCGCGTCCCTGCCGACAGGAAACAGGTGAGCA
GATTGCCATCAAGCAGTGCCGGCAGGAGCTCAGCCCCCGGAACCGAGAGCGGTGGT
GCCTGGAGATCCAGATCATGAGAAGGCTGACCCACCCCAATGTGGTGGCTGCCCGA
GATGTCCCTGAGGGGATGCAGAACTTGGCGCCCAATGACCTGCCCCTGCTGGCCATG
GAGTACTGCCAAGGAGGAGATCTCCGGAAGTACCTGAACCAGTTTGAGAACTGCTG
TGGTCTGCGGGAAGGTGCCATCCTCACCTTGCTGAGTGACATTGCCTCTGCGCTTAG
ATACCTTCATGAAAACAGAATCATCCATCGGGATCTAAAGCCAGAAAACATCGTCCT
GCAGCAAGGAGAACAGAGGTTAATACACAAAATTATTGACCTAGGATATGCCAAGG
AGCTGGATCAGGGCAGTCTTTGCACATCATTCGTGGGGACCCTGCAGTACCTGGCCC
CAGAGCTACTGGAGCAGCAGAAGTACACAGTGACCGTCGACTACTGGAGCTTCGGC
ACCCTGGCCTTTGAGTGCATCACGGGCTTCCGGCCCTTCCTCCCCAACTGGCAGCCC
GTGCAGTGGCATTCAAAAGTGCGGCAGAAGAGTGAGGTGGACATTGTTGTTAGCGA
AGACTTGAATGGAACGGTGAAGTTTTCAAGCTCTTTACCCTACCCCAATAATCTTAA
CAGTGTCCTGGCTGAGCGACTGGAGAAGTGGCTGCAACTGATGCTGATGTGGCACC
CCCGACAGAGGGGCACGGATCCCACGTATGGGCCCAATGGCTGCTTCAAGGCCCTG
GATGACATCTTAAACTTAAAGCTGGTTCATATCTTGAACATGGTCACGGGCACCATC
CACACCTACCCTGTGACAGAGGATGAGAGTCTGCAGAGCTTGAAGGCCAGAATCCA
ACAGGACACGGGCATCCCAGAGGAGGACCAGGAGCTGCTGCAGGAAGCGGGCCTG
GCGTTGATCCCCGATAAGCCTGCCACTCAGTGTATTTCAGACGGCAAGTTAAATGAG
GGCCACACATTGGACATGGATCTTGTTTTTCTCTTTGACAACAGTAAAATCACCTAT
GAGACTCAGATCTCCCCACGGCCCCAACCTGAAAGTGTCAGCTGTATCCTTCAAGAG
CCCAAGAGGAATCTCGCCTTCTTCCAGCTGAGGAAGGTGTGGGGCCAGGTCTGGCA
CAGCATCCAGACCCTGAAGGAAGATTGCAACCGGCTGCAGCAGGGACAGCGAGCCG
CCATGATGAATCTCCTCCGAAACAACAGCTGCCTCTCCAAAATGAAGAATTCCATGG
CTTCCATGTCTCAGCAGCTCAAGGCCAAGTTGGATTTCTTCAAAACCAGCATCCAGA
TTGACCTGGAGAAGTACAGCGAGCAAACCGAGTTTGGGATCACATCAGATAAACTG
CTGCTGGCCTGGAGGGAAATGGAGCAGGCTGTGGAGCTCTGTGGGCGGGAGAACGA
AGTGAAACTCCTGGTAGAACGGATGATGGCTCTGCAGACCGACATTGTGGACTTAC
AGAGGAGCCCCATGGGCCGGAAGCAGGGGGGAACGCTGGACGACCTAGAGGAGCA
AGCAAGGGAGCTGTACAGGAGACTAAGGGAAAAACCTCGAGACCAGCGAACTGAG
GGTGACAGTCAGGAAATGGTACGGCTGCTGCTTCAGGCAATTCAGAGCTTCGAGAA
GAAAGTGCGAGTGATCTATACGCAGCTCAGTAAAACTGTGGTTTGCAAGCAGAAGG
CGCTGGAACTGTTGCCCAAGGTGGAAGAGGTGGTGAGCTTAATGAATGAGGATGAG
AAGACTGTTGTCCGGCTGCAGGAGAAGCGGCAGAAGGAGCTCTGGAATCTCCTGAA
GATTGCTTGTAGCAAGGTCCGTGGTCCTGTCAGTGGAAGCCCGGATAGCATGAATGC
CTCTCGACTTAGCCAGCCTGGGCAGCTGATGTCTCAGCCCTCCACGGCCTCCAACAG
CTTACCTGAGCCAGCCAAGAAGAGTGAAGAACTGGTGGCTGAAGCACATAACCTCT
GCACCCTGCTAGAAAATGCCATACAGGACACTGTGAGGGAACAAGACCAGAGTTTC
ACGGCCCTAGACTGGAGCTGGTTACAGACGGAAGAAGAAGAGCACAGCTGCCTGGA
GCAGGCCTCATGA
Human IκB CDS
(SEQ ID NO: 12)
ATGTTCCAGGCGGCCGAGCGCCCCCAGGAGTGGGCCATGGAGGGCCCCCGCGACGG
GCTGAAGAAGGAGCGGCTACTGGACGACCGCCACGACAGCGGCCTGGACTCCATGA
AAGACGAGGAGTACGAGCAGATGGTCAAGGAGCTGCAGGAGATCCGCCTCGAGCC
GCAGGAGGTGCCGCGCGGCTCGGAGCCCTGGAAGCAGCAGCTCACCGAGGACGGG
GACTCGTTCCTGCACTTGGCCATCATCCATGAAGAAAAGGCACTGACCATGGAAGTG
ATCCGCCAGGTGAAGGGAGACCTGGCCTTCCTCAACTTCCAGAACAACCTGCAGCA
GACTCCACTCCACTTGGCTGTGATCACCAACCAGCCAGAAATTGCTGAGGCACTTCT
GGGAGCTGGCTGTGATCCTGAGCTCCGAGACTTTCGAGGAAATACCCCCCTACACCT
TGCCTGTGAGCAGGGCTGCCTGGCCAGCGTGGGAGTCCTGACTCAGTCCTGCACCAC
CCCGCACCTCCACTCCATCCTGAAGGCTACCAACTACAATGGCCACACGTGTCTACA
CTTAGCCTCTATCCATGGCTACCTGGGCATCGTGGAGCTTTTGGTGTCCTTGGGTGCT
GATGTCAATGCTCAGGAGCCCTGTAATGGCCGGACTGCCCTTCACCTCGCAGTGGAC
CTGCAAAATCCTGACCTGGTGTCACTCCTGTTGAAGTGTGGGGCTGATGTCAACAGA
GTTACCTACCAGGGCTATTCTCCCTACCAGCTCACCTGGGGCCGCCCAAGCACCCGG
ATACAGCAGCAGCTGGGCCAGCTGACACTAGAAAACCTTCAGATGCTGCCAGAGAG
TGAGGATGAGGAGAGCTATGACACAGAGTCAGAGTTCACGGAGTTCACAGAGGACG
AGCTGCCCTATGATGACTGTGTGTTTGGAGGCCAGCGTCTGACGTT ATGA
Human IRAK CDS
(SEQ ID NO: 13)
ATGGCCGGGGGGCCGGGCCCGGGGGAGCCCGCAGCCCCCGGCGCCCAGCACTTCTT
GTACGAGGTGCCGCCCTGGGTCATGTGCCGCTTCTACAAAGTGATGGACGCCCTGGA
GCCCGCCGACTGGTGCCAGTTCGCCGCCCTGATCGTGCGCGACCAGACCGAGCTGC
GGCTGTGCGAGCGCTCCGGGCAGCGCACGGCCAGCGTCCTGTGGCCCTGGATCAAC
CGCAACGCCCGTGTGGCCGACCTCGTGCACATCCTCACGCACCTGCAGCTGCTCCGT
GCGCGGGACATCATCACAGCCTGGCACCCTCCCGCCCCGCTTCCGTCCCCAGGCACC
ACTGCCCCGAGGCCCAGCAGCATCCCTGCACCCGCCGAGGCCGAGGCCTGGAGCCC
CCGGAAGTTGCCATCCTCAGCCTCCACCTTCCTCTCCCCAGCTTTTCCAGGCTCCCAG
ACCCATTCAGGGCCTGAGCTCGGCCTGGTCCCAAGCCCTGCTTCCCTGTGGCCTCCA
CCGCCATCTCCAGCCCCTTCTTCTACCAAGCCAGGCCCAGAGAGCTCAGTGTCCCTC
CTGCAGGGAGCCCGCCCCTTTCCGTTTTGCTGGCCCCTCTGTGAGATTTCCCGGGGC
ACCCACAACTTCTCGGAGGAGCTCAAGATCGGGGAGGGTGGCTTTGGGTGCGTGTA
CCGGGCGGTGATGAGGAACACGGTGTATGCTGTGAAGAGGCTGAAGGAGAACGCTG
ACCTGGAGTGGACTGCAGTGAAGCAGAGCTTCCTGACCGAGGTGG
AGCAGCTGTCCAGGTTTCGTCACCCAAACATTGTGGACTTTGCTGGCTACTGTGCTC
AGAACGGCTTCTACTGCCTGGTGTACGGCTTCCTGCCCAACGGCTCCCTGGAGGACC
GTCTCCACTGCCAGACCCAGGCCTGCCCACCTCTCTCCTGGCCTCAGCGACTGGACA
TCCTTCTGGGTACAGCCCGGGCAATTCAGTTTCTACATCAGGACAGCCCCAGCCTCA
TCCATGGAGACATCAAGAGTTCCAACGTCCTTCTGGATGAGAGGCTGACACCCAAG
CTGGGAGACTTTGGCCTGGCCCGGTTCAGCCGCTTTGCCGGGTCCAGCCCCAGCCAG
AGCAGCATGGTGGCCCGGACACAGACAGTGCGGGGCACCCTGGCCTACCTGCCCGA
GGAGTACATCAAGACGGGAAGGCTGGCTGTGGACACGGACACCTTCAGCTTTGGGG
TGGTAGTGCTAGAGACCTTGGCTGGTCAGAGGGCTGTGAAGACGCACGGTGCCAGG
ACCAAGTATCTGAAAGACCTGGTGGAAGAGGAGGCTGAGGAGGCTGGAGTGGCTTT
GAGAAGCACCCAGAGCACACTGCAAGCAGGTCTGGCTGCAGATGCCTGGGCTGCTC
CCATCGCCATGCAGATCTACAAGAAGCACCTGGACCCCAGGCCCGGGCCCTGCCCA
CCTGAGCTGGGCCTGGGCCTGGGCCAGCTGGCCTGCTGCTGCCTGCACCGCCGGGCC
AAAAGGAGGCCTCCTATGACCCAGGTGTACGAGAGGCTAGAGAAGCTGCAGGCAGT
GGTGGCGGGGGTGCCCGGGCATTCGGAGGCCGCCAGCTGCATCCCCCCTTCCCCGC
AGGAGAACTCCTACGTGTCCAGCACTGGCAGAGCCCACAGTGGGGCTGCTCCATGG
CAGCCCCTGGCAGCGCCATCAGGAGCCAGTGCCCAGGCAGCAGAGCAGCTGCAGAG
AGGCCCCAACCAGCCCGTGGAGAGTGACGAGAGCCTAGGCGGCCTCTCTGCTGCCC
TGCGCTCCTGGCACTTGACTCCAAGC
TGCCCTCTGGACCCAGCACCCCTCAGGGAGGCCGGCTGTCCTCAGGGGGACACGGC
AGGAGAATCGAGCTGGGGGAGTGGCCCAGGATCCCGGCCCACAGCCGTGGAAGGA
CTGGCCCTTGGCAGCTCTGCATCATCGTCGTCAGAGCCACCGCAGATTATCATCAAC
CCTGCCCGACAGAAGATGGTCCAGAAGCTGGCCCTGTACGAGGATGGGGCCCTGGA
CAGCCTGCAGCTGCTGTCGTCCAGCTCCCTCCCAGGCTTGGGCCTGGAACAGGACAG
GCAGGGGCCCGAAGAAAGTGATGAATTTCAGAGCTGA
Human JNK CDS
(SEQ ID NO: 14)
ATGAGCAGAAGCAAGCGTGACAACAATTTTTATAGTGTAGAGATTGGAGATTCTAC
ATTCACAGTCCTGAAACGATATCAGAATTTAAAACCTATAGGCTCAGGAGCTCAAG
GAATAGTATGCGCAGCTTATGATGCCATTCTTGAAAGAAATGTTGCAATCAAGAAGC
TAAGCCGACCATTTCAGAATCAGACTCATGCCAAGCGGGCCTACAGAGAGCTAGTT
CTTATGAAATGTGTTAATCACAAAAATATAATTGGCCTTTTGAATGTTTTCACACCAC
AGAAATCCCTAGAAGAATTTCAAGATGTTTACATAGTCATGGAGCTCATGGATGCAA
ATCTTTGCCAAGTGATTCAGATGGAGCTAGATCATGAAAGAATGTCCTACCTTCTCT
ATCAGATGCTGTGTGGAATCAAGCACCTTCATTCTGCTGGAATTATTCATCGGGACT
TAAAGCCCAGTAATATAGTAGTAAAATCTGATTGCACTTTGAAGATTCTTGACTTCG
GTCTGGCCAGGACTGCAGGAACGAGTTTTATGATGACGCCTTATGTAGTGACTCGCT
ACTACAGAGCACCCGAGGTCATCCTTGGCATGGGCTACAAGGAAAACGTTGACATT
TGGTCAGTTGGGTGCATCATGGGAGAAATGATCAAAGGTGGTGTTTTGTTCCCAGGT
ACAGATCATATTGATCAGTGGAATAAAGTTATTGAACAGCTTGGAACACCATGTCCT
GAATTCATGAAGAAACTGCAACCAACAGTAAGGACTTACGTTGAAAACAGACCTAA
ATATGCTGGATATAGCTTTGAGAAACTCTTCCCTGATGTCCTTTTCCCAGCTGACTCA
GAACACAACAAACTTAAAGCCAGTCAGGCAAGGGATTTGTTATCCAAAATGCTGGT
AATAGATGCATCTAAAAGGATCTCTGTAGATGAAGCTCTCCAACACCCGTACATCAA
TGTCTGGTATGATCCTTCTGAAGCAGAAGCTCCACCACCAAAGATCCCTGACAAGCA
GTTAGATGAAAGGGAACACACAATAGAAGAGTGGAAAGAATTGATATATAAGGAA
GTTATGGACTTGGAGGAGAGAACCAAGAATGGAGTTATACGGGGGCAGCCCTCTCC
TTTAGGTGCAGCAGTGATCAATGGCTCTCAGCATCCATCATCATCGTCGTCTGTCAA
TGATGTGTCTTCAATGTCAACAGATCCGACTTTGGCCTCTGATACAGACAGCAGTCT
AGAAGCAGCAGCTGGGCCTCTGGGCTGCTGTAGATGA
Human LBP CDS
(SEQ ID NO: 15)
ATGGGGGCCTTGGCCAGAGCCCTGCCGTCCATACTGCTGGCATTGCTGCTTACGTCC
ACCCCAGAGGCTCTGGGTGCCAACCCCGGCTTGGTCGCCAGGATCACCGACAAGGG
ACTGCAGTATGCGGCCCAGGAGGGGCTATTAGCTCTGCAGAGTGAGCTGCTCAGGA
TCACGCTGCCTGACTTCACCGGGGACTTGAGGATCCCCCACGTCGGCCGTGGGCGCT
ATGAGTTCCACAGCCTGAACATCCACAGCTGTGAGCTGCTTCACTCTGCGCTGAGGC
CTGTCCCTGGCCAGGGCCTGAGTCTCAGCA
TCTCCGACTCCTCCATCCGGGTCCAGGGCAGGTGGAAGGTGCGCAAGTCATTCTTCA
AACTACAGGGCTCCTTTGATGTCAGTGTCAAGGGCATCAGCATTTCGGTCAACCTCC
TGTTGGGCAGCGAGTCCTCCGGGAGGCCCACAGTTACTGCCTCCAGCTGCAGCAGTG
ACATCGCTGACGTGGAGGTGGACATGTCGGGAGACTTGGGGTGGCTGTTGAACCTCT
TCCACAACCAGATTGAGTCCAAGTTCCAGAAAGTACTGGAGAGCAGGATTTGCGAA
ATGATCCAGAAATCGGTGTCCTCCGATCTACAGCCTTATCTCCAAACTCTGCCAGTT
ACAACAGAGATTGACAGTTTCGCCGACATTGATTATAGCTTAGTGGAAGCCCCTCGG
GCAACAGCCCAGATGCTGGAGGTGATGTTTAAGGGTGAAATCTTTCATCGTAACCAC
CGTTCTCCAGTTACCCTCCTTGCTGCAGTCATGAGCCTTCCTGAGGAACACAACAAA
ATGGTCTACTTTGCCATCTCGGATTATGTCTTCAACACGGCCAGCCTGGTTTATCATG
AGGAAGGATATCTGAACTTCTCCATCACAGATGACATGATACCGCCTGACTCTAATA
TCCGACTGACCACCAAGTCCTTCCGACCCTTCGTCCCACGGTTAGCCAGGCTCTACC
CCAACATGAACCTGGAACTCCAGGGATCAGTGCCCTCTGCTCCGCTCCTGAACTTCA
GCCCTGGGAATCTGTCTGTGGACCCCTATATGGAGATAGATGCCTTTGTGCTCCTGC
CCAGCTCCAGCAAGGAGCCTGTCTTCCGGCTCAGTGTGGCCA
CTAATGTGTCCGCCACCTTGACCTTCAATACCAGCAAGATCACTGGGTTCCTGAAGC
CAGGAAAGGTAAAAGTGGAACTGAAAGAATCCAAAGTTGGACTATTCAATGCAGAG
CTGTTGGAAGCGCTCCTCAACTATTACATCCTTAACACCCTCTACCCCAAGTTCAAT
GATAAGTTGGCCGAAGGCTTCCCCCTTCCTCTGCTGAAGCGTGTTCAGCTCTACGAC
CTTGGGCTGCAGATCCATAAGGACTTCCTGTTCTTGGGTGCCAATGTCCAATACATG
AGAGTTTGA
Human MEK1 CDS
(SEQ ID NO: 16)
ATGCCCAAGAAGAAGCCGACGCCCATCCAGCTGAACCCGGCCCCCGACGGCTCTGC
AGTTAACGGGACCAGCTCTGCGGAGACCAACTTGGAGGCCTTGCAGAAGAAGCTGG
AGGAGCTAGAGCTTGATGAGCAGCAGCGAAAGCGCCTTGAGGCCTTTCTTACCCAG
AAGCAGAAGGTGGGAGAACTGAAGGATGACGACTTTGAGAAGATCAGTGAGCTGG
GGGCTGGCAATGGCGGTGTGGTGTTCAAGGTCTCCCACAAGCCTTCTGGCCTGGTCA
TGGCCAGAAAGCTAATTCATCTGGAGATCAAACCCGCAATCCGGAACCAGATCATA
AGGGAGCTGCAGGTTCTGCATGAGTGCAACTCTCCGTACATCGTGGGCTTCTATGGT
GCGTTCTACAGCGATGGCGAGATCAGTATCTGCATGGAGCACATGGATGGAGGTTCT
CTGGATCAAGTCCTGAAGAAAGCTGGAAGAATTCCTGAACAAATTTTAGGAAAAGT
TAGCATTGCTGTAATAAAAGGCCTGACATATCTGAGGGAGAAGCACAAGATCATGC
ACAGAGATGTCAAGCCCTCCAACATCCTAGTCAACTCCCGTGGGGAGATCAAGCTCT
GTGACTTTGGGGTCAGCGGGCAGCTCATCGACTCCATGGCCAACTCCTTCGTGGGC
ACAAGGTCCTACATGTCGCCAGAAAGACTCCAGGGGACTCATTACTCTGTGCAGTCA
GACATCTGGAGCATGGGACTGTCTCTGGTAGAGATGGCGGTTGGGAGGTATCCCATC
CCTCCTCCAGATGCCAAGGAGCTGGAGCTGATGTTTGGGTGCCAGGTGGAAGGAGA
TGCGGCTGAGACCCCACCCAGGCCAAGGACCCCCGGGAGGCCCCTTAGCTCATACG
GAATGGACAGCCGACCTCCCATGGCAATTTTTGAGTTGTTGGATTACATAGTCAACG
AGCCTCCTCCAAAACTGCCCAGTGGAGTGTTCAGTCTGGAATTTCAAGATTTTGTGA
ATAAATGCTTAATAAAAAACCCCGCAGAGAGAGCAGATTTGAAGCAACTCATGGTT
CATGCTTTTATCAAGAGATCTGATGCTGAGGAAGTGGATTTTGCAGGTTGGCTCTGC
TCCACCATCGGCCTTAACCAGCCCAGCACACCAACCCATGCTGCTGGCGTCTAA
Human MEK2 CDS
(SEQ ID NO: 17)
ATGCTGGCCCGGAGGAAGCCGGTGCTGCCGGCGCTCACCATCAACCCTACCATCGC
CGAGGGCCCATCCCCTACCAGCGAGGGCGCCTCCGAGGCAAACCTGGTGGACCTGC
AGAAGAAGCTGGAGGAGCTGGAACTTGACGAGCAGCAGAAGAAGCGGCTGGAAGC
CTTTCTCACCCAGAAAGCCAAGGTCGGCGAACTCAAAGACGATGACTTCGAAAGGA
TCTCAGAGCTGGGCGCGGGCAACGGCGGGGTGGTCACCAAAGTCCAGCACAGACCC
TCGGGCCTCATCATGGCCAGGAAGCTGATCCACCTTGAGATCAAGCCGGCCATCCG
GAACCAGATCATCCGCGAGCTGCAGGTCCTGCACGAATGCAACTCGCCGTACATCG
TGGGCTTCTACGGGGCCTTCTACAGTGACGGGGAGATCAGCATTTGCATGGAACACA
TGGACGGCGGCTCCCTGGACCAGGTGCTGAAAGAGGCCAAGAGGATTCCCGAGGAG
ATCCTGGGGAAAGTCAGCATCGCGGTTCTCCGGGGCTTGGCGTACCTCCGAGAGAA
GCACCAGATCATGCACCGAGATGTGAAGCCCTCCAACATCCTCGTGAACTCTAGAG
GGGAGATCAAGCTGTGTGACTTCGGGGTGAGCGGCCAGCTCATCGACTCCATGGCC
AACTCCTTCGTGGGCACGCGCTCCTACATGGCTCCGGAGCGGTTGCAGGGCACA
CATTACTCGGTGCAGTCGGACATCTGGAGCATGGGCCTGTCCCTGGTGGAGCTGGCC
GTCGGAAGGTACCCCATCCCCCCGCCCGACGCCAAAGAGCTGGAGGCCATCTTTGG
CCGGCCCGTGGTCGACGGGGAAGAAGGAGAGCCTCACAGCATCTCGCCTCGGCCGA
GGCCCCCCGGGCGCCCCGTCAGCGGTCACGGGATGGATAGCCGGCCTGCCATGGCC
ATCTTTGAACTCCTGGACTATATTGTGAACGAGCCACCTCCTAAGCTGCCCAACGGT
GTGTTCACCCCCGACTTCCAGGAGTTTGTCAATAAATGCCTCATCAAGAACCCAGCG
GAGCGGGCGGACCTGAAGATGCTCACAAACCACACCTTCATCAAGCGGTCCGAGGT
GGAAGAAGTGGATTTTGCCGGCTGGTTGTGTAAAACCCTGCGGCTGAACCAGCCCG
GCACACCCACGCGCACCGCCGTGTGA
Human MEK3 CDS
(SEQ ID NO: 18)
ATGTCCAAGCCACCCGCACCCAACCCCACACCCCCCCGGAACCTGGACTCCCGGAC
CTTCATCACCATTGGAGACAGAAACTTTGAGGTGGAGGCTGATGACTTGGTGACCAT
CTCAGAACTGGGCCGTGGAGCCTATGGGGTGGTAGAGAAGGTGCGGCACGCCCAGA
GCGGCACCATCATGGCCGTGAAGCGGATCCGGGCCACCGTGAACTCACAGGAGCAG
AAGCGGCTGCTCATGGACCTGGACATCAACATGCGCACGGTCGACTGTTTCTACACT
GTCACCTTCTACGGGGCACTATTCAGAGAGGGAGACGTGTGGATCTGCATGGAGCTC
ATGGACACATCCTTGGACAAGTTCTACCGGAAGGTGCTGGATAAAAACATGACAAT
TCCAGAGGACATCCTTGGGGAGATTGCTGTGTCTATCGTGCGGGCCCTGGAGCATCT
GCACAGCAAGCTGTCGGTGATCCACAGAGATGTGAAGCCCTCCAATGTCCTTATCAA
CAAGGAGGGCCATGTGAAGATGTGTGACTTTGGCATCAGTGGCTACTTGGTGGACTC
TGTGGCCAAGACGATGGATGCCGGCTGCAAGCCCTACATGGCCCCTGAGAGGATCA
ACCCAGAGCTGAACCAGAAGGGCTACAATGTCAAGTCCGACGTCTGGAGCCTGGGC
ATCACCATGATTGAGATGGCCATCCTGCGGTTCCCTTACGAGTCCTGGGGGACCCCG
TTCCAGCAGCTGAAGCAGGTGGTGGAGGAGCCGTCCCCCCAGCTCCCAGCCGACCG
TTTCTCCCCCGAGTTTGTGGACTTCACTGCTCAGTGCCTGAGGAAGAACCCCGCAGA
GCGTATGAGCTACCTGGAGCTGATGGAGCACCCCTTCTTCACCTTGCACAAAACCAA
GAAGACGGACATTGCTGCCTTCGTGAAGGAGATCCTGGGAGAAGACTCATAG
Human MEK6 CDS
(SEQ ID NO: 19)
ATGTCTCAGTCGAAAGGCAAGAAGCGAAACCCTGGCCTTAAAATTCCAAAAGAAGC
ATTTGAACAACCTCAGACCAGTTCCACACCACCTCGAGATTTAGACTCCAAGGCTTG
CATTTCTATTGGAAATCAGAACTTTGAGGTGAAGGCAGATGACCTGGAGCCTATAAT
GGAACTGGGACGAGGTGCGTACGGGGTGGTGGAGAAGATGCGGCACGTGCCCAGC
GGGCAGATCATGGCAGTGAAGCGGATCCGAGCCACAGTAAATAGCCAGGAACAGA
AACGGCTACTGATGGATTTGGATATTTCCATGAGGACGGTGGACTGTCCATTCACTG
TCACCTTTTATGGCGCACTGTTTCGGGAGGGTGATGTGTGGATCTGCATGGAGCTCA
TGGATACATCACTAGATAAATTCTACAAACAAGTTATTGATAAAGGCCAGACAATTC
CAGAGGACATCTTAGGGAAAATAGCAGTTTCTATTGTAAAAGCATTAGAACATTTAC
ATAGTAAGCTGTCTGTCATTCACAGAGACGTCAAGCCTTCTAATGTACTCATCAATG
CTCTCGGTCAAGTGAAGATGTGCGATTTTGGAATCAGTGGCTACTTGGTGGACTCTG
TTGCTAAAACAATTGATGCAGGTTGCAAACCATACATGGCCCCTGAAAGAATAAAC
CCAGAGCTCAACCAGAAGGGATACAGTGTGAAGTCTGACATTTGGAGTCTGGGCAT
CACGATGATTGAGTTGGCCATCCTTCGATTTCCCTATGATTCATGGGGAACTCCATTT
CAGCAGCTCAAACAGGTGGTAGAGGAGCCATCGCCACAACTCCCAGCAGACAAGTT
CTCTGCAGAGTTTGTTGACTTTACCTCACAGTGCTTAAAGAAGAATTCCAAAGAACG
GCCTACATACCCAGAGCTAATGCAACATCCATTTTTCACCCTACATGAATCCAAAGG
AACAGATGTGGCATCTTTTGTAAAACTGATTCTTGGAGACTAA
Human MEKK1 CDS
(SEQ ID NO: 20)
ATGGCGGCGGCGGCGGGGAATCGCGCCTCGTCGTCGGGATTCCCGGGCGCCAGGGC
TACGAGCCCTGAGGCAGGCGGCGGCGGAGGAGCCCTCAAGGCGAGCAGCGCGCCC
GCGGCTGCCGCGGGACTGCTGCGGGAGGCGGGCAGCGGGGGCCGCGAGCGGGCGG
ACTGGCGGCGGCGGCAGCTGCGCAAAGTGCGGAGTGTGGAGCTGGACCAGCTGCCT
GAGCAGCCGCTCTTCCTTGCCGCCTCACCGCCGGCCTCCTCGACTTCCCCGTCGCCG
GAGCCCGCGGACGCAGCGGGGAGTGGGACCGGCTTCCAGCCTGTGGCGGTGCCGCC
GCCCCACGGAGCCGCGAGCCGCGGCGGCGCCCACCTTACCGAGTCGGTGGCGGCGC
CGGACAGCGGCGCCTCGAGTCCCGCAGCGGCCGAGCCCGGGGAGAAGCGGGCGCC
CGCCGCCGAGCCGTCTCCTGCAGCGGCCCCCGCCGGTCGTGAGATGGAGAATAAAG
AAACTCTCAAAGGGTTGCACAAGATGGATGATCGTCCAGAGGAACGAATGATCAGG
GAGAAACTGAAGGCAACCTGTATGCCAGCCTGGAAGCACGAATGGTTGGAAAGGAG
AAATAGGCGAGGGCCTGTGGTGGTAAAACCAATCCCAGTTAAAGGAGATGGATCTG
AAATGAATCACTTAGCAGCTGAGTCTCCAGGAGAGGTCCAGGCAAGTGCGGCTTCA
CCAGCTTCCAAAGGCCGACGCAGTCCTTCTCCTGGCAACTCCCCATCAGGTCGCACA
GTGAAATCAGAATCTCCAGGAGTAAGGAGAAAAAGAGTTTCCCCAGTGCCTTTTCA
GAGTGGCAGAATCACACCACCCCGAAGAGCCCCTTCACCAGATGGCTTCTCACCAT
ATAGCCCTGAGGAAACAAACCGCCGTGTTAACAAAGTGATGCGGGCCAGACTGTAC
TTACTGCAGCAGATAGGGCCTAACTCTTTCCTGATTGGAGGAGACAGCCCAGACAAT
AAATACCGGGTGTTTATTGGGCCTCAGAACTGCAGCTGTGCACGTGGAACATTCTGT
ATTCATCTGCTATTTGTGATGCTCCGGGTGTTTCAACTAGAACCTTCAGACCCAATGT
TATGGAGAAAAACTTTAAAGAATTTTGAGGTTGAGAGTTTGTTCCAGAAATATCACA
GTAGGCGTAGCTCAAGGATCAAAGCTCCATCTCGTAACACCATCCAGAAGTTTGTTT
CACGCATGTCAAATTCTCATACATTGTCATCATCTAGTACTTCTACGTCTAGTTCAGA
AAACAGCATAAAGGATGAAGAGGAACAGATGTGTCCTATTTGCTTGTTGGGCATGC
TTGATGAAGAAAGTCTTACAGTGTGTGAAGACGGCTGCAGGAACAAGCTGCACCAC
CACTGCATGTCAATTTGGGCAGAAGAGTGTAGAAGAAATAGAGAACCTTTAATATG
TCCCCTTTGTAGATCTAAGTGGAGATCTCATGATTTCTACAGCCACGAGTTGTCAAG
TCCTGTGGATTCCCCTTCTTCCCTCAGAGCTGCACAGCAGCAAACCGTACAGCAGCA
GCCTTTGGCTGGATCACGAAGGAATCAAGAGAGCAATTTTAACCTTACTCATTATGG
AACTCAGCAAATCCCTCCTGCTTACAAAGATTTAGCTGAGCCATGGATTCAGGTGTT
TGGAATGGAACTCGTTGGCTGCTTATTTTCTAGAAACTGGAATGTGAGAGAGATGGC
CCTCAGGCGTCTTTCCCATGATGTCAGTGGGGCCCTGCTGTTGGCAAATGGGGAGAG
CACTGGAAATTCTGGGGGCAGCAGTGGAAGCAGCCCGAGTGGGGGAGCCACCAGTG
GGTCTTCCCAGACCAGTATCTCAGGAGATGTGGTGGAGGCATGCTGCAGCGTTCTGT
CAATGGTCTGTGCTGACCCTGTCTACAAAGTGTACGTTGCTGCTTTAAAAACATTGA
GAGCCATGCTGGTATATACTCCTTGCCACAGTTTAGCGGAAAGAATCAAACTTCAGA
GACTTCTCCAGCCAGTTGTAGACACCATCCTAGTCAAATGTGCAGATGCCAATAGCC
GCACAAGTCAGCTGTCCATATCAACACTGTTGGAACTGTGCAAAGGCCAAGCAGGA
GAGTTGGCAGTTGGCAGAGAAATACTAAAAGCTGGATCCATTGGTATTGGTGGTGTT
GATTATGTCTTAAATTGTATTCTTGGAAACCAAACTGAATCAAACAATTGGCAAGAA
CTTCTTGGCCGCCTTTGTCTTATAGATAGACTGTTGTTGGAATTTCCTGCTGAATTTT
ATCCTCATATTGTCAGTACTGATGTTTCACAAGCTGAGCCTGTTGAAATCAGGTATA
AGAAGCTGCTGTCCCTCTTAACCTTTGCTTTGCAGTCCATTGATAATTCCCACTCAAT
GGTTGGCAAACTTTCCAGAAGGATCTACTTGAGTTCTGCAAGAATGGTTACTACAGT
ACCCCATGTGTTTTCAAAACTGTTAGAAATGCTGAGTGTTTCCAGTTCCACTCACTTC
ACCAGGATGCGTCGCCGTTTGATGGCTATTGCAGATGAGGTGGAAATTGCCGAAGC
CATCCAGTTGGGCGTAGAAGACACTTTGGATGGTCAACAGGACAGCTTCTTGCAGGC
ATCTGTTCCCAACAACTATCTGGAAACCACAGAGAACAGTTCCCCTGAGTGCACAGT
CCATTTAGAGAAAACTGGAAAAGGATTATGTGCTACAAAATTGAGTGCCAGTTCAG
AGGACATTTCTGAGAGACTGGCCAGCATTTCAGTAGGACCTTCTAGTTCAACAACAA
CAACAACAACAACAACAGAGCAACCAAAGCCAATGGTTCAAACAAAAGGCAGACC
CCACAGTCAGTGTTTGAACTCCTCTCCTTTATCTCATCATTCCCAATTAATGTTTCCA
GCCTTGTCAACCCCTTCTTCTTCTACCCCATCTGTACCAGCTGGCACTGCAACAGATG
TCTCTAAGCATAGACTTCAGGGATTCATTCCCTGCAGAATACCTTCTGCATCTCCTCA
AACACAGCGCAAGTTTTCTCTACAATTCCACAGAAACTGTCCTGAAAACAAAGACTC
AGATAAACTTTCCCCAGTCTTTACTCAGTCAAGACCCTTGCCCTCCAGTAACATACA
CAGGCCAAAGCCATCTAGACCTACCCCAGGTAATACAAGTAAACAGGGAGATCCCT
CAAAAAATAGCATGACACTTGATCTGAACAGTAGTTCCAAATGTGATGACAGCTTTG
GCTGTAGCAGCAATAGTAGTAATGCTGTTATACCCAGTGACGAGACAGTGTTCACCC
CAGTAGAGGAGAAATGCAGATTAGATGTCAATACAGAGCTCAACTCCAGTATTGAG
GACCTTCTTGAAGCATCTATGCCTTCAAGTGATACAACAGTAACTTTTAAGTCAGAA
GTTGCTGTCCTGTCTCCTGAAAAGGCTGAAAATGATGATACCTACAAAGATGATGTG
AATCATAATCAAAAGTGCAAAGAGAAGATGGAAGCTGAAGAAGAAGAAGCTTTAG
CAATTGCCATGGCAATGTCAGCGTCTCAGGATGCCCTCCCCATAGTTCCTCAGCTGC
AGGTTGAAAATGGAGAAGATATCATCATTATTCAACAGGATACACCAGAGACTCTA
CCAGGACATACCAAAGCAAAACAACCGTATAGAGAAGACACTGAATGGCTGAAAG
GTCAACAGATAGGCCTTGGAGCATTTTCTTCTTGTTATCAGGCTCAAGATGTGGGAA
CTGGAACTTTAATGGCTGTTAAACAGGTGACTTATGTCAGAAACACATCTTCTGAGC
AAGAAGAAGTAGTAGAAGCACTAAGAGAAGAGATAAGAATGATGAGCCATCTGAA
TCATCCAAACATCATTAGGATGTTGGGAGCCACGTGTGAGAAGAGCAATTACAATCT
CTTCATTGAATGGATGGCAGGGGGATCGGTGGCTCATTTGCTGAGTAAATATGGAGC
CTTCAAAGAATCAGTAGTTATTAACTACACTGAACAGTTACTCCGTGGCCTTTCGTA
TCTCCATGAAAACCAAATCATTCACAGAGATGTCAAAGGTGCCAATTTGCTAATTGA
CAGCACTGGTCAGAGACTAAGAATTGCAGATTTTGGAGCTGCAGCCAGGTTGGCAT
CAAAAGGAACTGGTGCAGGAGAGTTTCAGGGACAATTACTGGGGACAATTGCATTT
ATGGCACCTGAGGTACTAAGAGGTCAACAGTATGGAAGGAGCTGTGATGTATGGAG
TGTTGGCTGTGCTATTATAGAAATGGCTTGTGCAAAACCACCATGGAATGCAGAAAA
ACACTCCAATCATCTTGCTTTGATATTTAAGATTGCTAGTGCAACTACTGCTCCATCG
ATCCCTTCACATTTGTCTCCTGGTTTACGAGATGTGGCTCTTCGTTGTTTAGAACTTC
AACCTCAGGACAGACCTCCATCAAGAGAGCTACTGAAGCATCCAGTCTTTCGTACTA
CATGGTAG
Human MEKK 3 CDS
(SEQ ID NO: 21)
ATGGACGAACAGGAGGCATTGAACTCAATCATGAACGATCTGGTGGCCCTCCAGAT
GAACCGACGTCACCGGATGCCTGGATATGAGACCATGAAGAACAAAGACACAGGTC
ACTCAAATAGGCAGAAAAAACACAACAGCAGCAGCTCAGCCCTTCTGAACAGCCCC
ACAGTAACAACAAGCTCATGTGCAGGGGCCAGTGAGAAAAAGAAATTTTTGAGTGA
CGTCAGAATCAAGTTCGAGCACAACGGGGAGAGGCGAATTATAGCGTTCAGCCGGC
CTGTGAAATATGAAGATGTGGAGCACAAGGTGACAACAGTATTTGGACAACCTCTT
GATCTACATTACATGAACAATGAGCTCTCCATCCTGCTGAAAAACCAAGATGATCTT
GATAAAGCAATTGACATTTTAGATAGAAGCTCAAGCATGAAAAGCCTTAGGATATT
GCTGTTGTCCCAGGACAGAAACCATAACAGTTCCTCTCCCCACTCTGGGGTGTCCAG
ACAGGTGCGGATCAAGGCTTCCCAGTCCGCAGGGGATATAAATACTATCTACCAGC
CCCCCGAGCCCAGAAGCAGGCACCTCTCTGTCAGCTCCCAGAACCCTGGCCGAAGC
TCACCTCCCCCTGGCTATGTTCCTGAGCGGCAGCAGCACATTGCCCGGCAGGGGTCC
TACACCAGCATCAACAGTGAGGGGGAGTTCATCCCAGAGACCAGCGAGCAGTGCAT
GCTGGATCCCCTGAGCAGTGCAGAAAATTCCTTGTCTGGAAGCTGCCAATCCTTGGA
CAGGTCAGCAGACAGCCCATCCTTCCGGAAATCACGAATGTCCCGTGCCCAGAGCTT
CCCTGACAACAGACAGGAATACTCAGATCGGGAAACTCAGCTTTATGACAAAGGGG
TCAAAGGTGGAACCTACCCCCGGCGCTACCACGTGTCTGTGCACCACAAGGACTAC
AGTGATGGCAGAAGAACATTTCCCCGAATACGGCGTCATCAAGGCAACTTGTTCACC
CTGGTGCCCTCCAGCCGCTCCCTGAGCACAAATGGCGAGAACATGGGTCTGGCTGTG
CAATACCTGGACCCCCGTGGGCGCCTGCGGAGTGCGGACAGCGAGAATGCCCTCTC
TGTGCAGGAGAGGAATGTGCCAACCAAGTCTCCCAGTGCCCCCATCAACTGGCGCC
GGGGAAAGCTCCTGGGCCAGGGTGCCTTCGGCAGGGTCTATTTGTGCTATGACGTGG
ACACGGGACGTGAACTTGCTTCCAAGCAGGTCCAATTTGATCCAGACAGTCCTGAGA
CAAGCAAGGAGGTGAGTGCTCTGGAGTGCGAGATCCAGTTGCTAAAGAACTTGCAG
CATGAGCGCATCGTGCAGTACTATGGCTGTCTGCGGGACCGCGCTGAGAAGACCCT
GACCATCTTCATGGAGTACATGCCAGGGGGCTCGGTGAAAGACCAGTTGAAGGCTT
ACGGTGCTCTGACAGAGAGCGTGACCCGAAAGTACACGCGGCAGATCCTGGAGGGC
ATGTCCTACCTGCACAGCAACATGATTGTTCACCGGGACATTAAGGGAGCCAACATC
CTCCGAGACTCTGCTGGGAATGTAAAGCTGGGGGACTTTGGGGCCAGCAAACGCCT
GCAGACGATCTGTATGTCGGGGACGGGCATGCGCTCCGTCACTGGCACACCCTACTG
GATGAGCCCTGAGGTGATCAGCGGCGAGGGCTATGGAAGGAAAGCAGACGTGTGG
AGCCTGGGCTGCACTGTGGTGGAGATGCTGACAGAGAAACCACCGTGGGCAGAGTA
TGAAGCTATGGCCGCCATCTTCAAGATTGCCACCCAGCCCACCAATCCTCAGCTGCC
CTCCCACATCTCTGAACATGGCCGGGACTTCCTGAGGCGCATTTTTGTGGAGGCTCG
CCAGAGACCTTCAGCTGAGGAGCTGCTCA
CACACCACTTTGCACAGCTCATGTACTGA
Human MEKK4 CDS
(SEQ ID NO: 22)
ATGAGAGAAGCCGCTGCCGCGCTGGTCCCTCCTCCCGCCTTTGCCGTCACGCCTGCC
GCCGCCATGGAGGAGCCGCCGCCACCGCCGCCGCCGCCACCACCGCCACCGGAACC
CGAGACCGAGTCAGAACCCGAGTGCTGCTTGGCGGCGAGGCAAGAGGGCACATTGG
GAGATTCAGCTTGCAAGAGTCCTGAATCTGATCTAGAAGACTTCTCCGATGAAACAA
ATACAGAGAATCTTTATGGTACCTCTCCCCCCAGCACACCTCGACAGATGAAACGCA
TGTCAACCAAACATCAGAGGAATAATGTGGGGAGGCCAGCCAGTCGGTCTAATTTG
AAAGAAAAAATGAATGCACCAAATCAGCCTCCACATAAAGACACTGGAAAAACAGT
GGAGAATGTGGAAGAATACAGCTATAAGCAGGAGAAAAAGATCCGAGCAGCTCTTA
GAACAACAGAGCGTGATCATAAAAAAAATGTACAGTGCTCATTCATGTTAGACTCA
GTGGGTGGATCTTTGCCAAAAAAATCAATTCCAGATGTGGATCTCAATAAGCCTTAC
CTCAGCCTTGGCTGTAGCAATGCTAAGCTTCCAGTATCTGTGCCCATGCCTATAGCC
AGACCTGCACGCCAGACTTCTAGGACTGACTGTCCAGCAGATCGTTTAAAGTTTTTT
GAAACTTTACGACTTTTGCTAAAGCTTACCTCAGTCTCAAAGAAAAAAGACAGGGA
GCAAAGAGGACAAGAAAATACGTCTGGTTTCTGGCTTAACCGATCTAACGAACTGA
TCTGGTTAGAGCTACAAGCCTGGCATGCAGGACGGACAATTAACGACCAGGACTTC
TTTTTATATACAGCCCGTCAAGCCATCCCAGATATTATTAATGAAATCCTTACTTTCA
AAGTCGACTATGGGAGCTTCGCCTTTGTTAGAGATAGAGCTGGTTTTAATGGTACTT
CAGTAGAAGGGCAGTGCAAAGCCACTCCTGGAACAAAGATTGTAGGTTACTCAACA
CATCATGAGCATCTCCAACGCCAGAGGGTCTCATTTGAGCAGGTAAAACGGATAAT
GGAGCTGCTAGAGTACATAGAAGCACTTTATCCATCATTGCAGGCTCTTCAGAAGGA
CTATGAAAAATATGCTGCAAAAGACTTCCAGGACAGGGTGCAGGCACTCTGTTTGTG
GTTAAACATCACAAAAGACTTAAATCAGAAATTAAGGATTATGGGCACTGTTTTGGG
CATCAAGAATTTATCAGACATTGGCTGGCCAGTGTTTGAAATCCCTTCCCCTCGACC
ATCCAAAGGTAATGAGCCGGAGTATGAGGGTGATGACACAGAAGGAGAATTAAAG
GAGTTGGAAAGTAGTACGGATGAGAGTGAAGAAGAACAAATCTCTGATCCTAGGGT
ACCGGAAATCAGACAGCCCATAGATAACAGCTTCGACATCCAGTCGCGGGACTGCA
TATCCAAGAAGCTTGAGAGGCTCGAATCTGAGGATGATTCTCTTGGCTGGGGAGCAC
CAGACTGGAGCACAGAAGCAGGCTTTAGTAGACATTGTCTGACTTCTATTTATAGAC
CATTTGTAGACAAAGCACTGAAGCAGATGGGGTTAAGAAAGTTAATTTTAAGACTTC
ACAAGCTAATGGATGGTTCCTTGCAAAGGGCACGTATAGCATTGGTAAAGAACGAT
CGTCCAGTGGAGTTTTCTGAATTTCCAGATCCCATGTGGGGTTCAGATTATGTGCAG
TTGTCAAGGACACCACCTTCATCTGAGGAGAAATGCAGTGCTGTGTCGTGGGAGGA
GCTGAAGGCCATGGATTTACCTTCATTCGAACCTGCCTTCCTAGTTCTCTGCCGAGTC
CTTCTGAATGTCATACATGAGTGTCTGAAGTTAAGATTGGAGCAGAGACCTGCTGGA
GAACCATCTCTCTTGAGTATTAAGCAGCTGGTGAGAGAGTGTAAGGAGGTCCTGAA
GGGCGG
CCTGCTGATGAAGCAGTACTACCAGTTCATGCTGCAGGAGGTTCTGGAGGACTTGGA
GAAGCCCGACTGCAACATTGACGCTTTTGAAGAGGATCTACATAAAATGCTTATGGT
GTATTTTGATTACATGAGAAGCTGGATCCAAATGCTACAGCAATTACCTCAAGCATC
GCATAGTTTAAAAAATCTGTTAGAAGAAGAATGGAATTTCACCAAAGAAATAACTC
ATTACATACGGGGAGGAGAAGCACAGGCCGGGAAGCTTTTCTGTGACATTGCAGGA
ATGCTGCTGAAATCTACAGGAAGTTTTTTAGAATTTGGCTTACAGGAGAGCTGTGCT
GAATTTTGGACTAGTGCGGATGACAGCAGTGCTTCCGACGAAATCAGGAGGTCTGTT
ATAGAGATCAGTCGAGCCCTGAAGGAGCTCTTCCATGAAGCCAGAGAAAGGGCTTC
CAAAGCACTTGGATTTGCTAAAATGTTGAGAAAGGACCTGGAAATAGCAGCAGAAT
TCAGGCTTTCAGCCCCAGTTAGAGACCTCCTGGATGTTCTGAAATCAAAACAGTATG
TCAAGGTGCAAATTCCTGGGTTAGAAAACTTGCAAATGTTTGTTCCAGACACTCTTG
CTGAGGAGAAGAGTATTATTTTGCAGTTACTCAATGCAGCTGCAGGAAAGGACTGTT
CAAAAGATTCAGATGACGTACTCATCGATGCCTATCTGCTTCTGACCAAGCACGGTG
ATCGAGCCCGTGATTCAGAGGACAGCTGGGGCACCTGGGAGGCACAGCCTGTCAAA
GTCGTGCCTCAGGTGGAGACTGTTGACACCCTGAGAAGCATGCAGGTGGATAATCTT
TTACTAGTTGTCATGCAGTCTGCGCATCTCACAATTCAGAGAAAAGCTTTCCAGCAG
TCCATTGAGGGACTTATGACTCTGTGCCAGGAGCAGACATCCAGTCAGCCGGTCATC
GCCAAAGCTTTGCAGCAGCTGAAGAATGATGCATTGGAGCTATGCAACAGGATAAG
CAATGCCATTGACCGCGTGGACCACATGTTCACATCAGAATTTGATGCTGAGGTTGA
TGAATCTGAATCTGTCACCTTGCAACAGTACTACCGAGAAGCAATGATTCAGGGGTA
CAATTTTGGATTTGAGTATCATAAAGAAGTTGTTCGTTTGATGTCTGGGGAGTTTAG
ACAGAAGAT
AGGAGACAAATATATAAGCTTTGCCCGGAAGTGGATGAATTATGTCCTGACTAAAT
GTGAGAGTGGTAGAGGTACAAGACCCAGGTGGGCGACTCAAGGATTTGATTTTCTA
CAAGCAATTGAACCTGCCTTTATTTCAGCTTTACCAGAAGATGACTTCTTGAGTTTAC
AAGCCTTGATGAATGAATGCATTGGCCATGTCATAGGAAAACCACACAGTCCTGTTA
CAGGTTTGTACCTTGCCATTCATCGGAACAGCCCCCGTCCTATGAAGGTACCTCGAT
GCCATAGTGACCCTCCTAACCCACACCTCATTATCCCCACTCCAGAGGGATTCAGCA
CTCGGAGCATGCCTTCCGACGCGCGGAGCCATGGCAGCCCTGCTGCTGCTGCTGCTG
CTGCTGCTGCTGCTGTTGCTGCCAGTCGGCCCAGCCCCTCTGGTGGTGACTCTGTGCT
GCCCAAATCCATCAGCAGTGCCCATGATACCAGGGGTTCCAGCGTTCCTGAAAATG
ATCGATTGGCTTCCATAGCTGCTGAATTGCAGTTTAGGTCCCTGAGTCGTCACTCAA
GCCCCACGGAGGAGCGAGATGAACCAGCATATCCAAGAGGAGATTCAAGTGGGTCC
ACAAGAAGAAGTTGGGAACTTCGGACACTAATCAGCCAGAGTAAAGATACTGCTTC
TAAACTAGGACCCATAGAAGCTATCCAGAAGTCAGTCCGATTGTTTGAAGAAAAGA
GGTACCGAGAAATGAGGAGAAAGAATATCATTGGTCAAGTTTGTGATACGCCTAAG
TCCTATGATAATGTTATGCACGTTGGCTTGAGGAAGGTGACCTTCAAATGGCAAAGA
GGAAACAAAATTGGAGAAGGCCAGTATGGGAAGGTGTACACCTGCATCAGCGTCGA
CACCGGGGAGCTGATGGCCATGAAAGAGATTCGATTTCAACCTAATGACCATAAGA
CTATCAAGGAAACTGCAGACGAATTGAAAATATTCGAAGGCATCAAACACCCCAAT
CTGGTTCGGTATTTTGGTGTGGAGCTCCATAGAGAAGAAATGTACATCTTCATGGAG
TACTGCGATGAGGGGACTTTAGAAGAGGTGTCAAGGCTGGGACTTCAGGAACATGT
GATTAGGCTGTATTCAAAGCAGATCACCATTGCGATCAACGTCCTCCATGAGCATGG
CATAGTCCACCGTGACATTAAAGGTGCCAATATCTTCCTTACCTCATCTGGATTAATC
AAACTGGGAGATTTTGGATGTTCAGTAAAGCTCAAAAACAATGCCCAGACCATGCC
TGGTGAAGTGAACAGCACCCTGGGGACAGCAGCATACATGGCACCTGAAGTCATCA
CTCGTGCCAAAGGAGAGGGCCATGGGCGTGCGGCCGACATCTGGAGTCTGGGGTGT
GTTGTCATAGAGATGGTGACTGGCAAGAGGCCTTGGCATGAGTATGAGCACAACTTT
CAAATTATGTATAAAGTGGGGATGGGACATAAGCCACCAATCCCTGAAAGATTAAG
CCCTGAAGGAAAGGACTTCCTTTCTCACTGCCTTGAGAGTGACCCAAAGATGAGATG
GACCGCCAGCCAGCTCCTCGACCATTCGTTTGTCAA
GGTTTGCACAGATGAAGAATG
Human MEKK 6 CDS
(SEQ ID NO: 23)
ATGGCGGGGCCGTGTCCCCGGTCCGGGGCGGAGCGCGCCGGCAGCTGCTGGCAGGA
CCCGCTGGCCGTGGCGCTGAGCCGGGGCCGGCAGCTCGCGGCGCCCCCGGGCCGGG
GCTGCGCGCGGAGCCGGCCGCTCAGCGTGGTCTACGTGCTGACCCGGGAGCCGCAG
CCCGGGCTCGAGCCTCGGGAGGGAACCGAGGCGGAGCCGCTGCCCCTGCGCTGCCT
GCGCGAGGCTTGCGCGCAGGTCCCCCGGCCGCGGCCGCCCCCGCAGCTGCGCAGCC
TGCCCTTCGGGACGCTGGAGCTAGGCGACACCGCGGCTCTGGATGCCTTCTACAACG
CGGATGTGGTGGTGCTGGAGGTGAGCAGCTCGCTGGTACAGCCCTCCCTGTTCTACC
ACCTTGGTGTGCGTGAGAGCTTCAGCATGACCAACAATGTGCTCCTCTGCTCCCAGG
CCGACCTCCCTGACCTGCAGGCCCTGCGGGAGGATGTTTTCCAGAAGAACTCGGATT
GCGTTGGCAGCTACACACTGATCCCCTATGTGGTGACGGCCACTGGTCGGGTGCTGT
GTGGTGATGCAGGCCTTCTGCGGGGCCTGGCTGATGGGCTGGTACAGGCTGGAGTG
GGGACCGAGGCCCTGCTCACTCCCCTGGTGGGCCGGCTTGCCCGCCTGCTGGAGGCC
ACACCCACAGACTCTTGTGGCTATTTCCGGGAGACCATTCGGCGGGACATCCGGCAG
GCGCGGGAGCGGTTCAGTGGGCCACAGCTGCGGCAGGAGCTGGCTC
GCCTGCAGCGGAGACTGGACAGCGTGGAGCTGCTGAGCCCCGACATCATCATGAAC
TTGCTGCTCTCCTACCGCGATGTGCAGGACTACTCGGCCATCATTGAGCTGGTGGAG
ACGCTGCAGGCCTTGCCCACCTGTGATGTGGCCGAGCAGCATAATGTCTGCTTCCAC
TACACTTTTGCCCTCAACCGGAGGAACAGGCCTGGGGACCGGGCGAAGGCCCTGTC
TGTGCTGCTGCCGCTGGTACAGCTTGAGGGCTCTGTGGCGCCCGATCTGTACTGCAT
GTGTGGCCGTATCTACAAGGACATGTTCTTCAGCTCGGGTTTCCAGGATGCTGGGCA
CCGGGAGCAGGCCTATCACTGGTATCGCAAGGCTTTTGACGTAGAGCCCAGCCTTCA
CTCAGGCATCAATGCAGCTGTGCTCCTCATTGCTGCCGGGCAGCACTTTGAGGATTC
CAAAGAGCTCCGGCTAATAGGCATGAAGCTGGGCTGCCTGCTGGCCCGCAAAGGCT
GCGTGGAGAAGATGCAGTATTACTGGGATGTGGGTTTCTACCTGGGAGCCCAGATCC
TCGCCAATGACCCCACCCAGGTGGTGCTGGCTGCAGAGCAGCTGTATAAGCTCAAT
GCCCCCATATGGTACCTGGTGTCCGTGATGGAGACCTTCCTGCTCTACCAGCACTTC
AGGCCCACGCCAGAGCCCCCTGGAGGGCCACCACGCCGTGCCCACTTCTGGCTCCA
CTTCTTGCTACAGTCCTGCCAACCATTCAAGACAGCCTGTGCCCAGGGCGACCAGTG
CTTGGTGCTGGTCCTGGAGATGAACAAGGTGCTGCTGCCTGCAAAGCTCGAGGTTCG
GGGTACTGACCCAGTAAGCACAGTGACCCTGAGCCTGCTGGAGCCTGAGACCCAGG
ACATTCCCTCCAGCTGGACCTTCCCAGTCGCCTCCATATGCGGAGTCAGCGCCTCAA
AGCGCGACGAGCGCTGCTGCTTCCTCTATGCACTCCCCCCGGCTCAGGACGTCCAGC
TGTGCTTCCCCAGCGTAGGGCACTGCCAGTGGTTCTGCGGCCTGATCCAGGCCTGGG
TGACGAACCCGGATTCCACGGCGCCCGCGGAGGAGGCGGAGGGCGCGGGGGAGAT
GTTGGAGTTTGATTATGA
GTACACGGAGACGGGCGAGCGGCTGGTGCTGGGCAAGGGCACGTATGGGGTGGTGT
ACGCGGGCCGCGATCGCCACACGAGGGTGCGCATCGCCATCAAGGAGATCCCGGAG
CGGGACAGCAGGTTCTCTCAGCCCCTGCATGAAGAGATCGCTCTTCACAGACGCCTG
CGCCACAAGAACATAGTGCGCTATCTGGGCTCAGCTAGCCAGGGCGGCTACCTTAA
GATCTTCATGGAGGAAGTGCCTGGAGGCAGCCTGTCCTCCTTGCTGCGGTCGGTGTG
GGGACCCCTGAAGGACAACGAGAGCACCATCAGTTTCTACACCCGCCAGATCCTGC
AGGGACTTGGCTACTTGCACGACAACCACATCGTGCACAGGGACATAAAAGGGGAC
AATGTGCTGATCAACACCTTCAGTGGGCTGCTCAAGATTTCTGACTTCGGCACCTCC
AAGCGGCTGGCAGGCATCACACCTTGCACTGAGACCTTCACAGGAACTCTGCAGTA
TATGGCCCCAGAAATCATTGACCAGGGCCCACGCGGGTATGGGAAAGCAGCTGACA
TCTGGTCACTGGGCTGCACTGTCATTGAGATGGCCACAGGTCGCCCCCCCTTCCACG
AGCTCGGGAGCCCACAGGCTGCCATGTTTCAGGTGGGTATGTACAAGGTCCATCCGC
CAATGCCCAGCTCTCTGTCGGCCGAGGCCCAAGCCTTTCTCCTCCGAACTTTTGAGC
CAGACCCCCGCCTCCGAGCCAGCGCCCAGACACTGCTGGGGGACCCCTTCCTGCAG
CCTGGGAAAAGGAGCCGCAGCCCCAGCTCCCCACGACATGCTCCACGGCCCTCAGA
TGCCCCTTCTGCCAGTCCCACTCCTTCAGCCAACTCAACCACCCAGTCTCAGACATTC
CCGTGCCCTCAGGCACCCTCTCAGCACCCACCCAGCCCCCCGAAGCGCTGCCTCAGT
TATGGGGGCACCAGCCAGCTCCGGGTGCCCGAGGAGCCTGCGGCCGAGGAGCCTGC
GTCTCCGGAGGAGAGTTCGGGGCTGAGCCTGCTGCACCAGGAGAGCAAGCGTCGGG
CCATGCTGGCCGCAGTATTGGAGCAGGAGCTGCCAGCGCTGGCGGAGAATCTGCAC
CAGGAGCAGAAGCAAGAGCAGGGGGCCCGTCTGGGCAGAAACCATGTGGAAGAGC
TGCTGCGCTGCCTCGGGGCACACATCCACACTCCCAACCGCCGGCAGCTCGCCCAGG
AGCTGCGGGCGCTGCAAGGACGGCTGAGGGCCCAGGGCCTTGGGCCTGCGCTTCTG
CACAGACCGCTGTTTGCCTTCCCGGATGCGGTGAAGCAGATCCTCCGCAAGCGCCAG
ATCCGTCCACACTGGATGTTCGTTCTGGACTCACTGCTCAGCCGTGCTGTGCGGGCA
GCCCTGGGTGTGCTAGGACCGGAGGTGGAGAAGGAGGCGGTCTCACCGAGGTCAGA
GGAGCTGAGTAATGAAGGGGACTCCCAGCAGAGCCCAGGCCAGCAGAGCCCGCTTC
CGGTGGAGCCCGAGCAGGGCCCCGCTCCTCTGATGGTGCAGCTGAGCCTCTTGAGG
GCAGAGACTGATCGGCTGCGCGAAATCCTGG
CGGGGAAGGAACGGGAGTACCAGGCCCTGGTGCAGCGGGCTCTACAGCGGCTGAAT
GAGGAAGCCCGGACCTATGTCCTGGCCCCAGAGCCTCCAACTGCTCTTTCAACGGAC
CAGGGCCTGGTGCAGTGGCTACAGGAACTGAATGTGGATTCAGGCACCATCCAAAT
GCTGTTGAACCATAGCTTCACCCTCCACACTCTGCTCACCTATGCCACTCGAGATGA
CCTCATCTACACCCGCATCAGGGGAGGGATGGTATGCCGCATCTGGAGGGCCATCTT
GGCACAGCGAGCAGGATCCACACCAGTCACCTCTGGACCCTGA
Human MEKK7 CDS
(SEQ ID NO: 24)
ATGTCTACAGCCTCTGCCGCCTCCTCCTCCTCCTCGTCTTCGGCCGGTGAGATGATCG
AAGCCCCTTCCCAGGTCCTCAACTTTGAAGAGATCGACTACAAGGAGATCGAGGTG
GAAGAGGTTGTTGGAAGAGGAGCCTTTGGAGTTGTTTGCAAAGCTAAGTGGAGAGC
AAAAGATGTTGCTATTAAACAAATAGAAAGTGAATCTGAGAGGAAAGCGTTTATTG
TAGAGCTTCGGCAGTTATCCCGTGTGAACCATCCTA
ATATTGTAAAGCTTTATGGAGCCTGCTTGAATCCAGTGTGTCTTGTGATGGAATATG
CTGAAGGGGGCTCTTTATATAATGTGCTGCATGGTGCTGAACCATTGCCATATTATA
CTGCTGCCCACGCAATGAGTTGGTGTTTACAGTGTTCCCAAGGAGTGGCTTATCTTC
ACAGCATGCAACCCAAAGCGCTAATTCACAGGGACCTGAAACCACCAAACTTACTG
CTGGTTGCAGGGGGGACAGTTCTAAAAATTTGTGATTTTGGTACAGCCTGTGACATT
CAGACACACATGACCAATAACAAGGGGAGTGCTGCTTGGATGGCACCTGAAGTTTT
TGAAGGTAGTAATTACAGTGAAAAATGTGACGTCTTCAGCTGGGGTATTATTCTTTG
GGAAGTGATAACGCGTCGGAAAC
CCTTTGATGAGATTGGTGGCCCAGCTTTCCGAATCATGTGGGCTGTTCATAATGGTA
CTCGACCACCACTGATAAAAAATTTACCTAAGCCCATTGAGAGCCTGATGACTCGTT
GTTGGTCTAAAGATCCTTCCCAGCGCCCTTCAATGGAGGAAATTGTGAAAATAATGA
CTCACTTGATGCGGTACTTTCCAGGAGCAGATGAGCCATTACAGTATCCTTGTCAGT
ATTCAGATGAAGGACAGAGCAACTCTGCCACCAGTACAGGCTCATTCATGGACATT
GCTTCTACAAATACGAGTAACAAAAGTGACACTAATATGGAGCAAGTTCCTGCCAC
AAATGATACTATTAAGCGCTTAGAATCAAAATTGTTGAAAAATCAGGCAAAGCAAC
AGAGTGAATCTGGACGTTTAAGCTTGGGAGCCTCCCGTGGGAGCAGTGTGGAGAGC
TTGCCCCCAACCTCTGAGGGCAAGAGGATGAGTGCTGACATGTCTGAAATAGAAGC
TAGGATCGCCGCAACCACAGGCAACGGACAGCCAAGACGTAGATCCATCCAAGACT
TGACTGTAACTGGAACAGAACCTGGTCAGGTGAGCAGTAGGTCATCCAGTCCCAGT
GTCAGAATGATTACTACCTCAGGACCAACCTCAGAAAAGCCAACTCGAAGTCATCC
ATGGACCCCTGATGATTCCACAGATACCAATGGATCAGATAACTCCATCCCAATGGC
TTATCTTACACTGGATCACCAACTACAGCCTCTAGCACCGTGCCCAAACTCCAAAGA
ATCTATGGCAGTGTTTGAACAGCATTGTAAAATGGCACAAGAATATATGAAAGTTCA
AACAGAAATTGCATTGTTATTACAGAGAAAGCAAGAACTAGTTGCAGAACTGGACC
AGGATGAAAAGGACCAGCAAAATACATCTCGCCTGGTACAGGAACATAAAAAGCTT
TTAGATGAAAACAAAAGCCTTTCTACTTACTACCAGCAATGCAAAAAACAACTAGA
GGTCATCAGAAGTCAGCAGCAGAAACGACAAGGCACTTCATGA
Human MK2 CDS
(SEQ ID NO: 25)
ATGCTGTCCAACTCCCAGGGCCAGAGCCCGCCGGTGCCGTTCCCCGCCCCGGCCCCG
CCGCCGCAGCCCCCCACCCCTGCCCTGCCGCACCCCCCGGCGCAGCCGCCGCCGCCG
CCCCCGCAGCAGTTCCCGCAGTTCCACGTCAAGTCCGGCCTGCAGATCAAGAAGAA
CGCCATCATCGATGACTACAAGGTCACCAGCCAGGTCCTGGGGCTGGGCATCAACG
GCAAAGTTTTGCAGATCTTCAACAAGAGGACCCAGGAGAAATTCGCCCTCAAAATG
CTTCAGGACTGCCCCAAGGCCCGCAGGGAGGTGGAGCTGCACTGGCGGGCCTCCCA
GTGCCCGCACATCGTACGGATCGTGGATGTGTACGAGAATCTGTACGCAGGGAGGA
AGTGCCTGCTGATTGTCATGGAATGTTTGGACGGTGGAGAACTCTTTAGCCGAATCC
AGGATCGAGGAGACCAGGCATTCACAGAAAGAGAAGCATCCGAAATCATGAAGAG
CATCGGTGAGGCCATCCAGTATCTGCATTCAATCAACATTGCCCATCGGGATGTCAA
GCCTGAGAATCTCTTATACACCTCCAAAAGGCCCAACGCCATCCTGAAACTCACTGA
CTTTGGCTTTGCCAAGGAAACCACCAGCCACAACTCTTTGACCACTCCTTGTTATAC
ACCGTACTATGTGGCTCCAGAAGTGCTGGGTCCAGAGAAGTATGACAAGTCCTGTG
ACATGTGGTCCCTGGGTGTCATCATGTACATCCTGCTGTGTGGGTATCCCCCCTTCTA
CTCCAACCACGGCCTTGCCATCTCTCCGGGCATGAAGACTCGCATCCGAATGGGCCA
GTATGAATTTCCCAACCCAGAATGGTCAGAAGTATCAGAGGAAGTGAAGATGCTCA
TTCGGAATCTGCTGAAAACAGAGCCCACCCAGAGAATGACCATCACCGAGTTTATG
AACCACCCTTGGATCATGCAATCAACAAAGGTCCCTCAAACCCCACTGCACACCAG
CCGGGTCCTGAAGGAGGACAAGGAGCGGTGGGAGGATGTCAAGGGGTGTCTTCATG
ACAAGAACAGCGACCAGGCCACTTGGCTGACCAGGTTGTGA
Human MyD88 CDS
(SEQ ID NO: 26)
ATGCGACCCGACCGCGCTGAGGCTCCAGGACCGCCCGCCATGGCTGCAGGAGGTCC
CGGCGCGGGGTCTGCGGCCCCGGTCTCCTCCACATCCTCCCTTCCCCTGGCTGCTCTC
AACATGCGAGTGCGGCGCCGCCTGTCTCTGTTCTTGAACGTGCGGACACAGGTGGCG
GCCGACTGGACCGCGCTGGCGGAGGAGATGGACTTTGAGTACTTGGAGATCCGGCA
ACTGGAGACACAAGCGGACCCCACTGGCAGGCTGCTGGACGCCTGGCAGGGACGCC
CTGGCGCCTCTGTAGGCCGACTGCTCGAGCTGCTTACCAAGCTGGGCCGCGACGACG
TGCTGCTGGAGCTGGGACCCAGCATTGAGGAGGATTGCCAAAAGTATATCTTGAAG
CAGCAGCAGGAGGAGGCTGAGAAGCCTTTACAGGTGGCCGCTGTAGACAGCAGTGT
CCCACGGACAGCAGAGCTGGCGGGCATCACCACACTTGATGACCCCCTGGGGCATA
TGCCTGAGCGTTTCGATGCCTTCATCTGCTATTGCCCCAGCGACATCCAGTTTGTGCA
GGAGATGATCCGGCAACTGGAACAGACAAACTATCGACTGAAGTTGTGTGTGTCTG
ACCGCGATGTCCTGCCTGGCACCTGTGTCTGGTCTATTGCTAGTGAGCTCATCGAAA
AGAGGTTGGCTAGAAGGCCACGGGGTGGGTGCCGCCGGATGGTGGTGGTTGTCTCT
GATGATTACCTGCAGAGCAAGGAATGTGACTTCCAGACCAAATTTGCACTCAGCCTC
TCTCCAGGTGCCCATCAGAAGCGACTGATCCCCATCAAGTACAAGGCAATGAAGAA
AGAGTTCCCCAGCATCCTGAGGTTCATCACTGTCTGCGACTACACCAACCCCTGCAC
CAAATCTTGGTTCTGGACTCGCCTTGCCAAGGCCTTGTCCCTGCCCTGA
Human NF-κB CDS
(SEQ ID NO: 27)
ATGGCAGAAGATGATCCATATTTGGGAAGGCCTGAACAAATGTTTCATTTGGATCCT
TCTTTGACTCATACAATATTTAATCCAGAAGTATTTCAACCACAGATGGCACTGCCA
ACAGATGGCCCATACCTTCAAATATTAGAGCAACCTAAACAGAGAGGATTTCGTTTC
CGTTATGTATGTGAAGGCCCATCCCATGGTGGACTACCTGGTGCCTCTAGTGAAAAG
AACAAGAAGTCTTACCCTCAGGTCAAAATCTGCAACTATGTGGGACCAGCAAAGGT
TATTGTTCAGTTGGTCACAAATGGAAAAAATATCCACCTGCATGCCCACAGCCTGGT
GGGAAAACACTGTGAGGATGGGATCTGCACTGTAACTGCTGGACCCAAGGACATGG
TGGTCGGCTTCGCAAACCTGGGTATACTTCATGTGACAAAGAAAAAAGTATTTGAAA
CACTGGAAGCACGAATGACAGAGGCGTGTATAAGGGGCTATAATCCTGGACTCTTG
GTGCACCCTGACCTTGCCTATTTGCAAGCAGAAGGTGGAGGGGACCGGCAGCTGGG
AGATCGGGAAAAAGAGCTAATCCGCCAAGCAGCTCTGCAGCAGACCAAGGAGATG
GACCTCAGCGTGGTGCGGCTCATGTTTACAGCTTTTCTTCCGGATAGCACTGGCAGC
TTCACAAGGCGCCTGGAACCCGTGGTATCAGACGCCATCTATGACAGTAAAGCCCC
CAATGCATCCAACTTGAAAATTGTAAGAATGGACAGGACAGCTGGATGTGTGACTG
GAGGGGAGGAAATTTATCTTCTTTGTGACAAAGTTCAGAAAGATGACATCCAGATTC
GATTTTATGAAGAGGAAGAAAATGGTGGAGTCTGGGAAGGATTTGGAGATTTTTCC
CCCACAGATGTTCATAGACAATTTGCCATTGTCTTCAAAACTCCAAAGTATAAAGAT
ATTAATATTACAAAACCAGCCTCTGTGTTTGTCCAGCTTCGGAGGAAATCTGACTTG
GAAACTAGTGAACCAAAACCTTTCCTCTACTATCCTGAAATCAAAGATAAAGAAGA
AGTGCAGAGGAAACGTCAGAAGCTCATGCCCAATTTTTCGGATAGTTTCGGCGGTGG
TAGTGGTGCTGGAGCTGGAGGCGGAGGCATGTTTGGTAGTGGCGGTGGAGGAGGGG
GCACTGGAAGTACAGGTCCAGGGTATAGCTTCCCACACTATGGATTTCCTACTTATG
GTGGGATTACTTTCCATCCTGGAACTACTAAATCTAATGCTGGGATGAAGCATGGAA
CCATGGACACTGAATCTAAAAAGGACCCTGAAGGTTGTGACAAAAGTGATGACAAA
AACACTGTAAACCTCTTTGGGAAAGTTATTGAAACCACAGAGCAAGATCAGGAGCC
CAGCGAGGCCACCGTTGGGAATGGTGAGGTCACTCTAACGTATGCAACAGGAACAA
AAGAAGAGAGTGCTGGAGTTCAGGATAACCTCTTTCTAGAGAAGGCTATGCAGCTT
GCAAAGAGGCATGCCAATGCCCTTTTCGACTACGCGGTGACAGGAGACGTGAAGAT
GCTGCTGGCCGTCCAGCGCCATCTCACTGCTGTGCAGGATGAGAATGGGGACAGTGT
CTTACACTTAGCAATCATCCACCTTCATTCTCAACTTGTGAGGGATCTACTAGAAGTC
ACATCTGGTTTGATTTCTGATGACATTATCAACATGAGAAATGATCTGTACCAGACG
CCCTTGCACTTGGCAGTGATCACTAAGCAGGAAGATGTGGTGGAGGATTTGCTGAG
GGCTGGGGCCGACCTGAGCCTTCTGGACCGCTTGGGTAACTCTGTTTTGCACCTAGC
TGCCAAAGAAGGACATGATAAAGTTCTCAGTATCTTACTCAAGCACAAAAAGGCAG
CACTACTTCTTGACCACCCCAACGGGGACGGTCTGAATGCCATTCATCTAGCCATGA
TGAGCAATAGCCTGCCATGTTTGCTGCTGCTGGTGGCCGCTGGGGCTGACGTCAATG
CTCAGGAGCAGAAGTCCGGGCGCACAGCACTGCACCTGGCTGTGGAGCACGACAAC
ATCTCATTGGCAGGCTGCCTGCTCCTGGAGGGTGATGCCCATGTGGACAGTACTACC
TACGATGGAACCACACCCCTGCATATAGCAGCTGGGAGAGGGTCCACCAGGCTGGC
AGCTCTTCTCAAAGCAGCAGGAGCAGATCCCCTGGTGGAGAACTTTGAGCCTCTCTA
TGACCTGGATGACTCTTGGGAAAATGCAGGAGAGGATGAAGGAGTTGTGCCTGGAA
CCACGCCTCTAGATATGGCCACCAGCTGGCAGGTATTTGACATATTAAATGGGAAAC
CATATGAGCCAGAGTTTACATCTGATGATTTACTAGCACAAGGAGACATGAAACAG
CTGGCTGAAGATGTGAAGCTGCAGCTGTATAAGTTACTAGAAATTCCTGATCCAGAC
AAAAACTGGGCTACTCTGGCGCAGAAATTAGGTCTGGGGATACTTAATAATGCCTTC
CGGCTGAGTCCTGCTCCTTCCAAAACACTTATGGACAACTATGAGGTCTCTGGGGGT
ACAGTCAGAGAGCTGGTGGAGGCCCTGAGACAAATGGGCTACACCGAAGCAATTGA
AGTGATCCAGGCAGCCTCCAGCCCAGTGAAGACCACCTCTCAGGCCCACTCGCTGCC
TCTCTCGCCTGCCTCCACAAGGCAGCAAATAGACGAGCTCCGAGACAGTGACAGTG
TCTGCGACAGCGGCGTGGAGACATCCTTCCGCAAACTCAGCTTTACCGAGTCTCTGA
CCAGTGGTGCCTCACTGCTAACTCTCAACAAAATGCCCCATGATTATGGGCAGGAAG
GACCTCTAGAAGGCAAAATTTAG
Human NIK CDS
(SEQ ID NO: 28)
ATGGCAGTGATGGAAATGGCCTGCCCAGGTGCCCCTGGCTCAGCAGTGGGGCAGCA
GAAGGAACTCCCCAAAGCCAAGGAGAAGACGCCGCCACTGGGGAAGAAACAGAGC
TCCGTCTACAAGCTTGAGGCCGTGGAGAAGAGCCCTGTGTTCTGCGGAAAGTGGGA
GATCCTGAATGACGTGATTACCAAGGGCACAGCCAAGGAAGGCTCCGAGGCAGGGC
CAGCTGCCATCTCTATCATCGCCCAGGCTGAGTGTGAGAATAGCCAAGAGTTCAGCC
CCACCTTTTCAGAACGCATTTTCATCGCTGGGTCCAAACAGTACAGCCAGTCCGAGA
GTCTTGATCAGATCCCCAACAATGTGGCCCATGCTACAGAGGGCAAAATGGCCCGT
GTGTGTTGGAAGGGAAAGCGTCGCAGCAAAGCCCGGAAGAAACGGAAGAAGAAGA
GCTCAAAGTCCCTGGCTCATGCAGGAGTGGCCTTGGCCAAACCCCTCCCCAGGACCC
CTGAGCAGGAGAGCTGCACCATCCCAGTGCAGGAGGATGAGTCTCCACTCGGCGCC
CCATATGTTAGAAACACCCCGCAGTTCACCAAGCCTCTGAAGGAACCAGGCCTTGG
GCAACTCTGTTTTAAGCAGCTTGGCGAGGGCCTACGGCCGGCTCTGCCTCGATCAGA
ACTCCACAAACTGATCAGCCCCTTGCAATGTCTGAACCACGTGTGGAAACTGCACCA
CCCCCAGGACGGAGGCCCCCTGCCCCTGCCCACGCACCCCTTCCCCTATAGCAGACT
GCCTCATCCCTTCCCATTCCACCCTCTCCAGCCCTGGAAACCTCACCCTCTGGAGTCC
TTCCTGGGCAAACTGGCCTGTGTAGACAGCCAGAAACCCTTGCCTGACCCACACCTG
AGCAAACTGGCCTGTGTAGACAGTCCAAAGCCCCTGCCTGGCCCACACCTGGAGCC
CAGCTGCCTGTCTCGTGGTGCCCATGAGAAGTTTTCTGTGGAGGAATACCTAGTGCA
TGCTCTGCAAGGCAGCGTGAGCTCAGGCCAGGCCCACAGCCTGACCAGCCTGGCCA
AGACCTGGGCAGCAAGGGGCTCCAGATCCCGGGAGCCCAGCCCCAAAACTGAGGAC
AACGAGGGTGTCCTGCTCACTGAGAAACTCAAGCCAGTGGATTATGAGTACCGAGA
AGAAGTCCACTGGGCCACGCACCAGCTCCGCCTGGGCAGAGGCTCCTTCGGAGAGG
TGCACAGGATGGAGGACAAGCAGACTGGCTTCCAGTGCGCTGTCAAAAAGGTGCGG
CTGGAAGTATTTCGGGCAGAGGAGCTGATGGCATGTGCAGGATTGACCTCACCCAG
AATTGTCCCTTTGTATGGAGCTGTGAGAGAAGGGCCTTGGGTCAACATCTTCATGGA
GCTGCTGGAAGGTGGCTCCCTGGGCCAGCTGGTCAAGGAGCAGGGCTGTCTCCCAG
AGGACCGGGCCCTGTACTACCTGGGCCAGGCCCTGGAGGGTCTG
GAATACCTCCACTCACGAAGGATTCTGCATGGGGACGTCAAAGCTGACAACGTGCT
CCTGTCCAGCGATGGGAGCCACGCAGCCCTCTGTGACTTTGGCCATGCTGTGTGTCT
TCAACCTGATGGCCTGGGAAAGTCCTTGCTCACAGGGGACTACATCCCTGGCACAG
AGACCCACATGGCTCCGGAGGTGGTGCTGGGCAGGAGCTGCGACGCCAAGGTGGAT
GTCTGGAGCAGCTGCTGTATGATGCTGCACATGCTCAACGGCTGCCACCCCTGGACT
CAGTTCTTCCGAGGGCCGCTCTGCCTCAAGATTGCCAGCGAGCCTCCGCCTGTGAGG
GAGATCCCACCCTCCTGCGCCCCTCTCACAGCCCAGGCCATCCAAGAGGGGCTGAG
GAAAGAGCCCATCCACCGCGTGTCTGCAGCGGAGCTGGGAGGGAAGGTGAACCGG
GCACTACAGCAAGTGGGAGGTCTGAAGAGCCCTTGGAGGGGAGAATATAAAGAACC
AAGACATCCACCGCCAAATCAAGCCAATTACCACCAGACCCTCCATGCCCAGCCGA
GAGAGCTTTCGCCAAGGGCCCCAGGGCCCCGGCCAGCTGAGGAGACAACAGGCAG
AGCCCCTAAGCTCCAGCCTCCTCTCCCACCAGAGCCCCCAGAGCCAAACAAGTCTCC
TCCCTTGACTTTGAGCAAGGAGGAGTCTGGGATGTGGGAACCCTTACCTCTGTCCTC
CCTGGAGCCAGCCCCTGCCAGAAACCCCAGCTCACCAGAGCGGAAAGCAACCGTCC
CGGAGCAGGAACTGCAGCAGCTGGAAATAGAATTATTCCTCAACAGCCTGTCCCAG
CCATTTTCTCTGGAGGAGCAGGAGCAAATTCTCTCGTGCCTCAGCATCGACAGCCTC
TCCCTGTCGGATGACAGTGAGAAGAACCCATCAAAGGCCTCTCAAAGCTCGCGGGA
CACCCTGAGCTCAGGCGTACACTCCTGGAGCAGCCAGGCCGAGGCTCGAAGCTCCA
GCTGGAACATGGTGCTGGCCCGGGGGCGGCCCACCGACACCCCAAGCTATTTCAAT
GGTGTGAAAGTCCAAATACAGTCTCTTAATGGTGAACACCTGCACATCCGGGAGTTC
CACCGGGTCAAAGTGGGAGACATCGCCACTGGCATCAGCAGCCAGATCCCAGCTGC
AGCCTTCAGCTTGGTCACCAAAGACGGGCAGCCTGTTCGCTACGACATGGAGGTGC
CAGACTCGGGCATCGACCTGCAGTGCACACTGGCCCCTGATGGCAGCTTCGCCTGGA
GCTGGAGGGTCAAGCATGGCCAGCTGGAGAACAGGCCCTAA
Human p38 CDS
(SEQ ID NO: 29)
ATGTCTCAGGAGAGGCCCACGTTCTACCGGCAGGAGCTGAACAAGACAATCTGGGA
GGTGCCCGAGCGTTACCAGAACCTGTCTCCAGTGGGCTCTGGCGCCTATGGCTCTGT
GTGTGCTGCTTTTGACACAAAAACGGGGTTACGTGTGGCAGTGAAGAAGCTCTCCAG
ACCATTTCAGTCCATCATTCATGCGAAAAGAACCTACAGAGAACTGCGGTTACTTAA
ACATATGAAACATGAAAATGTGATTGGTCTGTTGGACGTTTTTACACCTGCAAGGTC
TCTGGAGGAATTCAATGATGTGTATCTGGTGACCCATCTCATGGGGGCAGATCTGAA
CAACATTGTGAAATGTCAGAAGCTTACAGATGACCATGTTCAGTTCCTTATCTACCA
AATTCTCCGAGGTCTAAAGTATATACATTCAGCTGACATAATTCACAGGGACCTAAA
ACCTAGTAATCTAGCTGTGAATGAAGACTGTGAGCTGAAGATTCTGGATTTTGGACT
GGCTCGGCACACAGATGATGAAATGACAGGCTACGTGGCCACTAGGTGGTACAGGG
CTCCTGAGATCATGCTGAACTGGATGCATTACAACCAGACAGTTGATATTTGGTCAG
TGGGATGCATAATGGCCGAGCTGTTGACTGGAAGAACATTGTTTCCTGGTACAGACC
ATATTAACCAGCTTCAGCAGATTATGCGTCTGACAGGAACACCCCCCGCTTATCTCA
TTAACAGGATGCCAAGCCATGAGGCAAGAAACTATATTCAGTCTTTGACTCAGATGC
CGAAGATGAACTTTGCGAATGTATTTATTGGTGCCAATCCCCTGGCTGTCGACTTGC
TGGAGAAGATGCTTGTATTGGACTCAGATAAGAGAATTACAGCGGCCCAAGCCCTT
GCACATGCCTACTTTGCTCAGTACCACGATCCTGATGATGAACCAGTGGCCGATCCT
TATGATCAGTCCTTTGAAAGCAGGGACCTCCTTATAGATGAGTGGAAAAGCCTGACC
TATGATGAAGTCATCAGCTTTGTGCCACCACCCCTTGACCAAGAAGAGATGGAGTCC
TGA
Human PKR CDS
(SEQ ID NO: 30)
ATGGCTGGTGATCTTTCAGCAGGTTTCTTCATGGAGGAACTTAATACATACCG
TCAGAAGCAGGGAGTAGTACTTAAATATCAAGAACTGCCTAATTCAGGACCTCCAC
ATGATAGGAGGTTTACATTTCAAGTTATAATAGATGGAAGAGAATTTCCAGAAGGT
GAAGGTAGATCAAAGAAGGAAGCAAAAAATGCCGCAGCCAAATTAGCTGTTGAGAT
ACTTAATAAGGAAAAGAAGGCAGTTAGTCCTTTATTATTGACAACAACGAATTCTTC
AGAAGGATTATCCATGGGGAATTACATAGGCCTTATCAATAGAATTGCCCAGAAGA
AAAGACTAACTGTAAATTATGAACAGTGTGCATCGGGGGTGCATGGGCCAGAAGGA
TTTCATTATAAATGCAAAATGGGACAGAAAGAATATAGTATTGGTACAGGTTCTACT
AAACAGGAAGCAAAACAATTGGCCGCTAAACTTGCATATCTTCAGATATTATCAGA
AGAAACCTCAGTGAAATCTGACTACCTGTCCTCTGGTTCTTTTGCTACTACGTGTGAG
TCCCAAAGCAACTCTTTAGTGACCAGCACACTCGCTTCTGAATCATCATCTGAAGGT
GACTTCTCAGCAGATACATCAGAGATAAATTCTAACAGTGACAGTTTAAACAGTTCT
TCGTTGCTTATGAATGGTCTCAGAAATAATCAAAGGAAGGCAAAAAGATCTTTGGC
ACCCAGATTTGACCTTCCTGACATGAAAGAAACAAAGTATACTGTGGACAAGAGGT
TTGGCATGGATTTTAAAGAAATAGAATTAATTGGCTCAGGTGGATTTGGCCAAGTTT
TCAAAGCAAAACACAGAATTGACGGAAAGACTTACGTTATTAAACGTGTTAAATAT
AATAACGAGAAGGCGGAGCGTGAAGTAAAAGCATTGGCAAAACTTGATCATGTAAA
TATTGTTCACTACAATGGCTGTTGGGATGGATTTGATTATGATCCTGAGACCAGTGA
TGATTCTCTTGAGAGCAGTGATTATGATCCTGAGAACAGCAAAAATAGTTCAAGGTC
AAAGACTAAGTGCCTTTTCATCCAAATGGAATTCTGTGATAAAGGGACCTTGGAACA
ATGGATTGAAAAAAGAAGAGGCGAGAAACTAGACAAAGTTTTGGCTTTGGAACTCT
TTGAACAAATAACAAAAGGGGTGGATTATATACATTCAAAAAAATTAATTCATAGA
GATCTTAAGCCAAGTAATATATTCTTAGTAGATACAAAACAAGTAAAGATTGGAGA
CTTTGGACTTGTAACATCTCTGAAAAATGATGGAAAGCGAACAAGGAGTAAGGGAA
CTTTGCGATACATGAGCCCAGAACAGATTTCTTCGCAAGACTATGGAAAGGAAGTG
GACCTCTACGCTTTGGGGCTAATTCTTGCTGAACTTCTTCATGTATGTGACACTGCTT
TTGAAACATCAAAGTTTTTCACAGACCTACGGGATGGCATCATCTCAGATATATTTG
ATAAAAAAGAAAAAACTCTTCTACAGAAATTACTCTCAAAGAAACCTGAGGATCGA
CCTAACACATCTGAAATACTAAGGACCTTGACTGTGTGGAAGAAAAGCCCAGAGAA
AAATGAACGACACACATGTTAG
Human Rac CDS
(SEQ ID NO: 31)
ATGAGCGACGTGGCTATTGTGAAGGAGGGTTGGCTGCACAAACGAGGGGAGTACAT
CAAGACCTGGCGGCCACGCTACTTCCTCCTCAAGAATGATGGCACCTTCATTGGCTA
CAAGGAGCGGCCGCAGGATGTGGACCAACGTGAGGCTCCCCTCAACAACTTCTCTG
TGGCGCAGTGCCAGCTGATGAAGACGGAGCGGCCCCGGCCCAACACCTTCATCATC
CGCTGCCTGCAGTGGACCACTGTCATCGAACGCACCTTCCATGTGGAGACTCCTGAG
GAGCGGGAGGAGTGGACAACCGCCATCCAGACTGTGGCTGACGGCCTCAAGAAGCA
GGAGGAGGAGGAGATGGACTTCCGGTCGGGCTCACCCAGTGACAACTCAGGGGCTG
AAGAGATGGAGGTGTCCCTGGCCAAGCCCAAGCACCGCGTGACCATGAACGAGTTT
GAGTACCTGAAGCTGCTGGGCAAGGGCACTTTCGGCAAGGTGATCCTGGTGAAGGA
GAAGGCCACAGGCCGCTACTACGCCATGAAGATCCTCAAGAAGGAAGTCATCGTGG
CCAAGGACGAGGTGGCCCACACACTCACCGAGAACCGCGTCCTGCAGAACTCCAGG
CACCCCTTCCTCACAGCCCTGAAGTACTCTTTCCAGACCCACGACCGCCTCTGCTTTG
TCATGGAGTACGCCAACGGGGGCGAGCTGTTCTTCCACCTGTCCCGGGAGCGTGTGT
TCTCCGAGGACCGGGCCCGCTTCTATGGCGCTGAGATTGTGTCAGCCCTGGACTACC
TGCACTCGGAGAAGAACGTGGTGTACCGGGACCTCAAGCTGGAGAACCTCATGCTG
GACAAGGACGGGCACATTAAGATCACAGACTTCGGGCTGTGCAAGGAGGGGATCAA
GGACGGTGCCACCATGAAGACCTTTTGCGGCACACCTGAGTACCTGGCCCCCGAGG
TGCTGGAGGACAATGACTACGGCCGTGCAGTGGACTGGTGGGGGCTGGGCGTGGTC
ATGTACGAGATGATGTGCGGTCGCCTGCCCTTCTACAACCAGGACCATGAGAAGCTT
TTTGAGCTCATCCTCATGGAGGAGATCCGCTTCCCGCGCACGCTTGGTCCCGAGGCC
AAGTCCTTGCTTTCAGGG
CTGCTCAAGAAGGACCCCAAGCAGAGGCTTGGCGGGGGCTCCGAGGACGCCAAGG
AGATCATGCAGCATCGCTTCTTTGCCGGTATCGTGTGGCAGCACGTGTACGAGAAGA
AGCTCAGCCCACCCTTCAAGCCCCAGGTCACGTCGGAGACTGACACCAGGTATTTTG
ATGAGGAGTTCACGGCCCAGATGATCACCATCACACCACCTGACCAAGATGACAGC
ATGGAGTGTGTGGACAGCGAGCGCAGGCCCCACTTCCCCCAGTTCTCCTACTCGGCC
AGCGGCACGGCCTGA
Human Raf CDS
(SEQ ID NO: 32)
ATGGCTAGCAAACGAAAATCTACAACTCCATGCATGGTTCGGACATCACAAGTAGT
AGAACAAGATGTGCCCGAGGAAGTAGACAGGGCCAAAGAGAAAGGAATCGGCACA
CCACAGCCTGACGTGGCCAAGGACAGTTGGGCAGCAGAACTTGAAAACTCTTCCAA
AGAAAACGAAGTGATAGAGGTGAAATCTATGGGGGAAAGCCAGTCCAAAAAACTC
CAAGGTGGTTATGAGTGCAAATACTGCCCCTACTCCACGCAAAACCTGAACGAGTTC
ACGGAGCATGTCGACATGCAGCATCCCAACGTGATTCTCAACCCCCTCTACGTGTGT
GCAGAATGTAACTTCACAACCAAAAAGTACGACTCCCTATCCGACCACAACTCCAA
GTTCCATCCCGGGGAGGCCAACTTCAAGCTGAAGTTAATTAAACGCAATAATCAAA
CTGTCTTGGAACAGTCCATCGAAACCACCAACCATGTCGTGTCCATCACCACCAGTG
GCCCTGGAACTGGTGACAGTGATTCTGGGATCTCGGTGAGTAAAACCCCCATCATGA
AGCCTGGAAAACCAAAAGCGGATGCCAAGAAGGTGCCCAAGAAGCCCGAGGAGAT
CACCCCCGAGAACCACGTGGAAGGGACCGCCCGCCTGGTGACAGACACAGCTGAGA
TCCTCTCGAGACTCGGCGGGGTGGAGCTCCTCCAAGACACATTAGGACACGTCATGC
CTTCTGTACAGCTGCCACCAAATATCAACCTTGTGCCCAAGGTCCCTGTCCCACTAA
ATACTACCAAATACAACTCTGCCCTGGATACAAATGCCACGATGATCAACTCTTTCA
ACAAGTTTCCTTACCCGACCCAGGCTGAGTTGTCCTGGCTGACAGCTGCCTCCAAAC
ACCCAGAGGAGCACATCAGAATCTGGTTTGCCACCCAGCGCTTAAAGCATGGCATC
AGCTGGTCCCCAGAAGAGGTGGAGGAGGCCCGGAAGAAGATGTTCAACGGCACCAT
CCAGTCAGTACCCCCGACCATCACTGTGCTGCCCGCCCAGTTGGCCCCCACAAAGGT
GACGCAGCCCATCCTCCAGACGGCTCTACCGTGCCAGATCCTCGGCCAGACTAGCCT
GGTGCTGACTCAGGTGACCAGCGGGTCAACAACCGTCTCTTGCTCCCCCATCACACT
TGCCGTGGCAGGAGTCACCAACCATGGCCAGAAGAGACCCTTGGTGACTCCCCAAG
CTGCCCCCGAACCCAAGCGTCCACACATCGCTCAGGTGCCAGAGCCCCCACCCAAG
GTGGCCAACCCCCCGCTCACACCAGCCAGTGACCGCAAGAAGACAAAGGAGCAGAT
AGCACATCTCAAGGCCAGCTTTCTCCAGAGCCAGTTCCCTGACGATGCCGAGGTTTA
CCGGCTCATCGAGGTGACTGGCCTTGCCAGGAGCGAGATCAAGAAGTGGTTCAGTG
ACCACCGATATCGGTGTCAAAGGGGCATCGTCCACATCACCAGCGAATCCCTTGCCA
AAGACCAGTTGGCCATCGCGGCCTCCCGACACGGTCGCACGTATCATGCGTACCCA
GACTTTGCCCCCCAGAAGTTCAAAGAGAAAACACAGGGTCAGGTTAAAATCTTGGA
AGACAGCTTTTTGAAAAGTTCTTTTCCTACCCAAGCAGAACTGGATCGGCTAAGGGT
GGAGACCAAGCTGAGCAGGAGAGAGATCGACTCCTGGTTCTCGGAGAGGCGGAAGC
TTCGAGACAGCATGGAACAAGCTGTCTTGGATTCCATGGGGTCTGGCAAAAAAGGC
CAAGATGTGGGAGCCCCCAATGGTGCTCTGTCTCGACTCGACCAGCTCTCCGGTGCC
CAGTTAACAAGTTCTCTGCCCAGCCCTTCGCCAGCAATTGCAAAAAGTCAAGAACA
GGTTCATCTCCTGAGGAGCACGTTTGCAAGAACCCAGTGGCCTACTCCCCAGGAGTA
CGACCAGTTAGCGGCCAAGACTGGCCTGGTCCGAACTGAGATTGTGCGTTGGTTCAA
GGAGAACAGATGCTTGCTGAAAACGGGAACCGTGAAGTGGATGGAGCAGTACCAGC
ACCAGCCCATGGCAGATGATCACGGCTACGATGCCGTAGCAAGGAAAGCAACAAAA
CCCATGGCCGAGAGCCCAAAGAACGGGGGTGATGTGGTTCCACAATATTACAAGGA
CCCCAAAAAGCTCTGCGAAGAGGACTTGGAGAAGTTGGTGACCAGGGTAAAAGTAG
GCAGCGAGCCAGCAAAAGACTGTTTGCCAGCAAAGCCCTCAGAGGCCACCTCAGAC
CGGTCAGAGGGCAGCAGCCGGGACGGCCAGGGTAGCGACGAGAACGAGGAGTCGA
GCGTTGTGGATTACGTGGAGGTGACGGTCGGGGAGGAGGATGCGATCTCAGATAGA
TCAGATAGCTGGAGTCAGGCTGCGGCAGAAGGTGTGTCGGAACTGGCTGAATCAGA
CTCCGACTGCGTCCCTGCAGAGGCTGGCCAGGCCTAG
Human K-Ras CDS
(SEQ ID NO: 33)
ATGACTGAATATAAACTTGTGGTAGTTGGAGCTGGTGGCGTAGGCAAGAGTGCCTTG
ACGATACAGCTAATTCAGAATCATTTTGTGGACGAATATGATCCAACAATAGAGGAT
TCCTACAGGAAGCAAGTAGTAATTGATGGAGAAACCTGTCTCTTGGATATTCTCGAC
ACAGCAGGTCAAGAGGAGTACAGTGCAATGAGGGACCAGTACATGAGGACTGGGG
AGGGCTTTCTTTGTGTATTTGCCATAAATAATACTAAATCATTTGAAGATATTCACCA
TTATAGAGAACAAATTAAAAGAGTTAAGGACTCTGAAGATGTACCTATGGTCCTAGT
AGGAAATAAATGTGATTTGCCTTCTAGAACAGTAGACACAAAACAGGCTCAGGACT
TAGCAAGAAGTTATGGAATTCCTTTTATTGAAACATCAGCAAAGACAAGACAGGGT
GTTGATGATGCCTTCTATACATTAGTTCGAGAAATTCGAAAACATAAAGAAAAGATG
AGCAAAGATGGTAAAAAGAAGAAAAAGAAGTCAAAGACAAAGTGTGTAATTATGT
AA
Human N-Ras CDS
(SEQ ID NO: 34)
ATGACTGAGTACAAACTGGTGGTGGTTGGAGCAGGTGGTGTTGGGAAAAGCGCACT
GACAATCCAGCTAATCCAGAACCACTTTGTAGATGAATATGATCCCACCATAGAGG
ATTCTTACAGAAAACAAGTGGTTATAGATGGTGAAACCTGTTTGTTGGACATACTGG
ATACAGCTGGACAAGAAGAGTACAGTGCCATGAGAGACCAATACATGAGGACAGG
CGAAGGCTTCCTCTGTGTATTTGCCATCAATAATAGCAAGTCATTTGCGGATATTAA
CCTCTACAGGGAGCAGATTAAGCGAGTAAAAGACTCGGATGATGTACCTATGGTGC
TAGTGGGAAACAAGTGTGATTTGCCAACAAGGACAGTTGATACAAAACAAGCCCAC
GAACTGGCCAAGAGTTACGGGATTCCATTCATTGAAACCTCAGCCAAGACCAGACA
GGGTGTTGAAGATGCTTTTTACACACTGGTAAGAGAAATACGCCAGTACCGAATGA
AAAAACTCAACAGCAGTGATGATGGGACTCAGGGTTGTATGGGATTGCCATGTGTG
GTGATGTAA
Human RIP CDS
(SEQ ID NO: 35)
ATGCAACCAGACATGTCCTTGAATGTCATTAAGATGAAATCCAGTGACTTCCTGGAG
AGTGCAGAACTGGACAGCGGAGGCTTTGGGAAGGTGTCTCTGTGTTTCCACAGAAC
CCAGGGACTCATGATCATGAAAACAGTGTACAAGGGGCCCAACTGCATTGAGCACA
ACGAGGCCCTCTTGGAGGAGGCGAAGATGATGAACAGACTGAGACACAGCCGGGTG
GTGAAGCTCCTGGGCGTCATCATAGAGGAAGGGAAGTACTCCCTGGTGATGGAGTA
CATGGAGAAGGGCAACCTGATGCACGTGCTGAAAGCCGAGATGAGTACTCCGCTTT
CTGTAAAAGGAAGGATAATTTTGGAAATCATTGAAGGAATGTGCTACTTACATGGA
AAAGGCGTGATACACAAGGACCTGAAGCCTGAAAATATCCTTGTTGATAATGACTTC
CACATTAAGATCGCAGACCTCGGCCTTGCCTCCTTTAAGATGTGGAGCAAACTGAAT
AATGAAGAGCACAATGAGCTGAGGGAAGTGGACGGCACCGCTAAGAAGAATGGCG
GCACCCTCTACTACATGGCGCCCGAGCACCTGAATGACGTCAACGCAAAGCCCACA
GAGAAGTCGGATGTGTACAGCTTTGCTGTAGTACTCTGGGCGATATTTGCAAATAAG
GAGCCATATGAAAATGCTATCTGTGAGCAGCAGTTGATAATGTGCATAAAATCTGG
GAACAGGCCAGATGTGGATGACATCACTGAGTACTGCCCAAGAGAAATTATCAGTC
TCATGAAGCTCTGCTGGGAAGCGAATCCGGAAGCTCGGCCGACATTTCCTGGCATTG
AAGAAAAATTTAGGCCTTTTTATTTAAGTCAATTAGAAGAAAGTGTAGAAGAGGAC
GTGAAGAGTTTAAAGAAAGAGTATTCAAACGAAAATGCAGTTGTGAAGAGAATGCA
GTCTCTTCAACTTGATTGTGTGGCAGTACCTTCAAGCCGGTCAAATTCAGCCACAGA
ACAGCCTGGTTCACTGCACAGTTCCCAGGGACTTGGGATGGGTCCTGTGGAGGAGTC
CTGGTTTGCTCCTTCCCTGGAGCACCCACAAGAAGAGAATGAGCCCAGCCTGCAGA
GTAAACTCCAAGACGAAGCCAACTACCATCTTTATGGCAGCCGCATGGACAGGCAG
ACGAAACAGCAGCCCAGACAGAATGTGGCTTACAACAGAGAGGAGGAAAGGAGAC
GCAGGGTCTCCCATGACCCTTTTGCACAGCAAAGACCTTACGAGAATTTTCAGAATA
CAGAGGGAAAAGGCACTGCTTATTCCAGTGCAGCCAGTCATGGTAATGCAGTGCAC
CAGCCCTCAGGGCTCACCAGCCAACCTCAAGTACTGTATCAGAACAATGGATTATAT
AGCTCACATGGCTTTGGAACAAGACCACTGGATCCAGGAACAGCAGGTCCCAGAGT
TTGGTACAGGCCAATTCCAAGTCATATGCCTAGTCTGCATAATATCCCAGTGCCTGA
GACCAACTATCTAGGAAATACACCCACCATGCCATTCAGCTCCTTGCCACCAACAGA
TGAATCTATAAAATATACCATATACAATAGTACTGGCATTCAGATTGGAGCCTACAA
TTATATGGAGATTGGTGGGACGAGTTCATCACTACTAGACAGCACAAATACGAACTT
CAAAGAAGAGCCAGCTGCTAAGTACCAAGCTATCTTTGATAATACCACTAGTCTGAC
GGATAAACACCTGGACCCAATCAGGGAAAATCTGGGAAAGCACTGGAAAAACTGTG
CCCGTAAACTGGGCTTCACACAGTCTCAGATTGATGAAATTGACCATGACTATGAGC
GAGATGGACTGAAAGAAAAGGTTTACCAGATGCTCCAAAAGTGGGTGATGAGGGAA
GGCATAAAGGGAGCCACGGTGGGGAAGCTGGCCCAGGCGCTCCACCAGTGTTCCAG
GATCGACCTTCTGAGCAGCTTGATTTACGTCAGCCAGAACTAA
Human TRAF6 CDS
(SEQ ID NO: 36)
ATGAGTCTGCTAAACTGTGAAAACAGCTGTGGATCCAGCCAGTCTGAAAGTGACTG
CTGTGTGGCCATGGCCAGCTCCTGTAGCGCTGTAACAAAAGATGATAGTGTGGGTGG
AACTGCCAGCACGGGGAACCTCTCCAGCTCATTTATGGAGGAGATCCAGGGATATG
ATGTAGAGTTTGACCCACCCCTGGAAAGCAAGTATGAATGCCCCATCTGCTTGATGG
CATTACGAGAAGCAGTGCAAACGCCATGCGGCCATAGGTTCTGCAAAGCCTGCATC
ATAAAATCAATAAGGGATGCAGGTCACAAATGTCCAGTTGACAATGAAATACTGCT
GGAAAATCAACTATTTCCAGACAATTTTGCAAAACGTGAGATTCTTTCTCTGATGGT
GAAATGTCCAAATGAAGGTTGTTTGCACAAGATGGAACTGAGACATCTTGAGGATC
ATCAAGCACATTGTGAGTTTGCTCTTATGGATTGTCCCCAATGCCAGCGTCCCTTCCA
AAAATTCCATATTAATATTCACATTCTGAAGGATTGTCCAAGGAGACAGGTTTCTTG
TGACAACTGTGCTGCATCAATGGCATTTGAAGATAAAGAGATCCATGACCAGAACT
GTCCTTTGGCAAATGTCATCTGTGAATACTGCAATACTATACTCATCAGAGAACAGA
TGCCTAATCATTATGATCTAGACTGCCCTACAGCCCCAATTCCATGCACATTCAGTA
CTTTTGGTTGCCATGAAAAGATGCAGAGGAATCACTTGGCACGCCACCTACAAGAG
AACACCCAGTCACACATGAGAATGTTGGCCCAGGCTGTTCATAGTTTGAGCGTTATA
CCCGACTCTGGGTATATCTCAGAGGTCCGGAATTTCCAGGAAACTATTCACCAGTTA
GAGGGTCGCCTTGTAAGACAAGACCATCAAATCCGGGAGCTGACTGCTAAAATGGA
AACTCAGAGTATGTATGTAAGTGAGCTCAAACGAACCATTCGAACCCTTGAGGACA
AAGTTGCTGAAATCGAAGCACAGCAGTGCAATGGAATTTATATTTGGAAGATTGGC
AACTTTGGAATGCATTTGAAATGTCAAGAAGAGGAGAAACCTGTTGTGATTCATAGC
CCTGGATTCTACACTGGCAAACCCGGGTACAAACTGTGCATGCGCTTGCACCTTCAG
TTACCGACTGCTCAGCGCTGTGCAAACTATATATCCCTTTTTGTCCACACAATGCAA
GGAGAATATGACAGCCACCTCCCTTGGCCCTTCCAGGGTACAATACGCCTTACAATT
CTTGATCAGTCTGAAGCACCTGTAAGGCAAAACCACGAAGAGATAATGGATGCCAA
ACCAGAGCTGCTTGCTTTCCAGCGACCCACAATCCCACGGAACCCAAAAGGTTTTGG
CTATGTAACTTTTATGCATCTGGAAGCCCTAAGACAAAGAACTTTCATTAAGGATGA
CACATTATTAGTGCGCTGTGAGGTCTCCACCCGCTTTGACATGGGTAGCCTTCGGAG
GGAGGGTTTTCAGCCACGAAGTACTGATGCAGGGGTATAG
Human TTP CDS
(SEQ ID NO: 37)
ATGGCCAACCGTTACACCATGGATCTGACTGCCATCTACGAGAGCCTCCTGTCGCTG
AGCCCTGACGTGCCCGTGCCATCCGACCATGGAGGGACTGAGTCCAGCCCAGGCTG
GGGCTCCTCGGGACCCTGGAGCCTGAGCCCCTCCGACTCCAGCCCGTCTGGGGTCAC
CTCCCGCCTGCCTGGCCGCTCCACCAGCCTAGTGGAGGGCCGCAGCTGTGGCTGGGT
GCCCCCACCCCCTGGCTTCGCACCGCTGGCTCCCCGCCTGGGCCCTGAGCTGTCACC
CTCACCCACTTCGCCCACTGCAACCTCCACCACCCCCTCGCGCTACAAGACTGAGCT
ATGTCGGACCTTCTCAGAGAGTGGGCGCTGCCGCTACGGGGCCAAGTGCCAGTTTGC
CCATGGCCTGGGCGAGCTGCGCCAGGCCAATCGCCACCCCAAATACAAGACGGAAC
TCTGTCACAAGTTCTACCTCCAGGGCCGCTGCCCCTACGGCTCTCGCTGCCACTTCAT
CCACAACCCTAGCGAAGACCTGGCGGCCCCGGGCCACCCTCCTGTGCTTCGCCAGA
GCATCAGCTTCTCCGGCCTGCCCTCTGGCCGCCGGACCTCACCACCACCACCAGGCC
TGGCCGGCCCTTCCCTGTCCTCCAGCTCCTTCTCGCCCTCCAGCTCCCCACCACCACC
TGGGGACCTTCCACTGTCACCCTCTGCCTTCTCTGCTGCCCCTGGCACCCCCCTGGCT
CGAAGAGACCCCACCCCAGTCTGTTGCCCCTCCTGCCGAAGGGCCACTCCTATCAGC
GTCTGGGGGCCCTTGGGTGGCCTGGTTCGGACCCCCTCTGTACAGTCCCTGGGATCC
GACCCTGATGAATATGCCAGCAGCGGCAGCAGCCTGGGGGGCTCTGACTCTCCCGT
CTTCGAGGCGGGAGTTTTTGCACCACCCCAGCCCGTGGCAGCCCCCCGGCGACTCCC
CATCTTCAATCGCATCTCTGTTTCTGAGTGA

An antisense nucleic acid molecule can be complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of SEQ ID NOs: 1-37). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEK1/2, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93.14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, RO5126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ 1S, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

### Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Preparative Examples

The following abbreviations have the indicated meanings.
ACN=acetonitrile
AcOH=acetic acid
AIBN=Azodiisobutyronitrile
9-BBN=9-borabicyclo[3.3.1]nonane
Boc₂O=Di-tert-butyl dicarbonate
(Bpin)₂=4,4,4′,4′,5,5,5′,5′-octamethyl-2,2′-bi(1,3,2-dioxaborolane)
CAN=Diammonium cerium(IV) nitrate
CCl₄=Perchloromethane
CHCl₃=Chloroform
ClSO₂OH=Chlorosulfonic acid
Conc.=Concentrated
Cs₂CO₃=Cesium carbonate
DAST=diethylaminosulfur trifluoride
DBU=1,8-diazabicycloundec-7-ene
DCM=dichloromethane
DEA=diethylamine
DMF=N,N-dimethylformamide
DMSO=dimethyl sulfoxide
DIEA=N,N-diisopropylethylamine
EtOH=ethanol
FA=formic acid
HCHO=Formaldehyde
Hex=hexane
HPLC=high performance liquid chromatography
IPA=propan-2-ol
LC-MS=liquid chromatography-mass spectrometry
LDA=Lithium diisopropylamide
Me=methyl
MeOH=methanol
NaBH₃CN=Sodium cyanoborohydride
NaSH=Sodium hydrosulfide
NBS=N-bromosuccinimide
n-BuLi=n-Butyllithium
NMR=nuclear magnetic resonance
PCl₅=Phosphorus pentachloride
Pd₂(dba)₃=tris(dibenzylideneacetone)dipalladium
Pd(dppf)Cl₂=dichloro[1,1′-bis(diphenylphosphino)ferrocene]palladium
Pd(PPh₃)₂Cl₂=Bis(triphenylphosphine)palladium(II) chloride
Ph=phenyl
PMB=p-methoxybenzyl
PPh₃Cl₂=dichlorotriphenylphosphorane
Rt=Retention time
RT=room temperature
SFC=supercritical fluid chromatogram
Sat.=saturated
TBAF=tetra-n-butylammonium fluoride
TBS=tert-butyldimethylsilyl
TBDPSCl=tert-butyldiphenylsilyl chloride
TBSCl=tert-butyldimethylsilyl chloride
t-BuOK=Potassium t-butoxide
t-BuONO=tert-Butyl nitrite
TEA=triethylamine
TFA=trifluoroacetic acid
THF=tetrahydrofuran
TLC=thin layer chromatography
UV=ultraviolet
X-phos=2-(Dicyclohexylphosphino)-2′,4′,6′-triisopropylbiphenyl

### General

The progress of reactions was often monitored by TLC or LC-MS. The identity of the products was often confirmed by LC-MS. The LC-MS was recorded using one of the following methods.

Method A: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.

Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with ACN and water (0.5% NH₄HCO₃), 2 minute total run time.

Method C: Shim-pack XR-ODS, C18, 3×50 mm, 2.5 um column, 1.0 uL injection, 1.5 mL/min flow rate, 90-900 amu scan range, 190400 nm UV range, 5-100% (2.1 min), 100% (0.6 min) gradient with ACN (0.05% TFA) and water (0.05% TFA), 3 minute total run time.

Method D: Kinetex EVO, C18, 3×50 mm, 2.2 um column, 1.0 uL injection, 1.5 mL/min flow rate, 90-900 amu scan range, 190400 nm UV range, 10-95% (2.1 min), 95% (0.6 min) gradient with ACN and water (0.5% NH₄HCO₃), 3 minute total run time.

The final targets were purified by Prep-HPLC. The Prep-HPLC was carried out using the following method.

Method E: Pre-HPLC. Column, XBridge Shield RP18 OBD (19×250 mm, 10 um); mobile phase, Water (10 mmol/L NH₄HCO₃) and ACN, UV detection 254/210 nm.

NMR was recorded on BRUKER NMR 300.03 MHz, DUL-C-H, ULTRASHIELD™300, AVANCE II 300 B-ACS™120 or BRUKER NMR 400.13 MHz, BBFO, 10 ULTRASHIELD™400, AVANCE III 400, B-ACS™120.

Racemic compounds of this invention can be resolved to give individual enantiomers using a variety of known methods. For example, chiral stationary phases can be used and the elution conditions can include normal phase or super-critical fluid with or without acidic or basic additives. Enantiomerically pure acids or bases can be used to form diatereomeric salts with the racemic compounds whereby pure enantiomers can be obtained by fractional crystallization. The racemates can also be derivatized with enantiomerically pure auxiliary reagents to form diastereomeric mixtures that can be separated. The auxiliary is then removed to give pure enantiomers.

Scheme of final targets: Schemes 1-6 illustrate several conditions used for coupling of acid 1 and sulfonimidoylamide 2 to afford acyl sulfonimidoylamide 3. As used in the schemes, rings “A” and “B” may be substituted as disclosed herein.

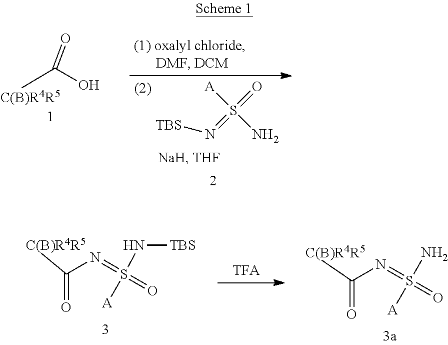

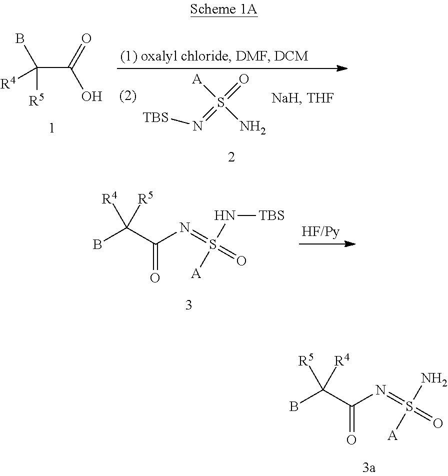

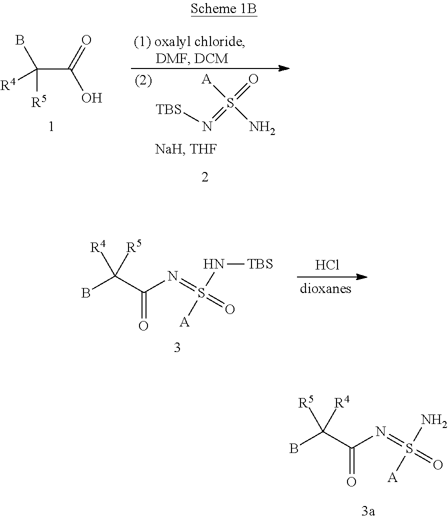

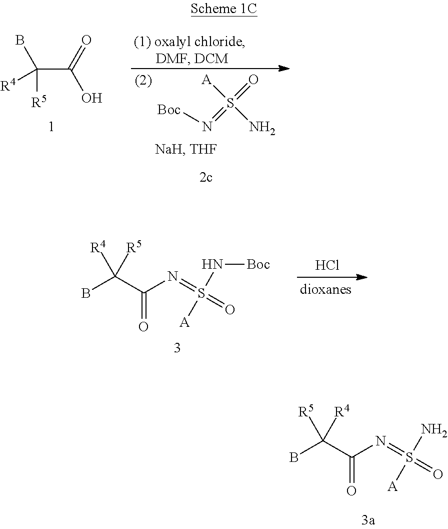

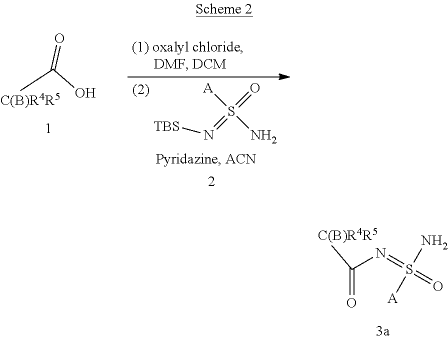

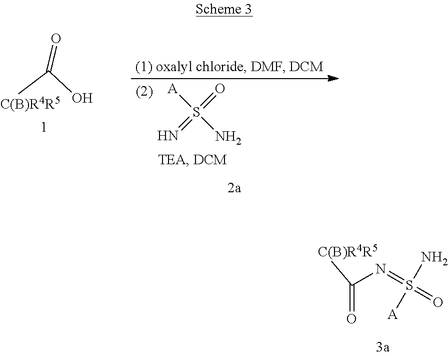

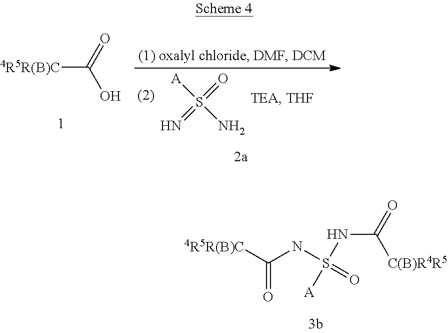

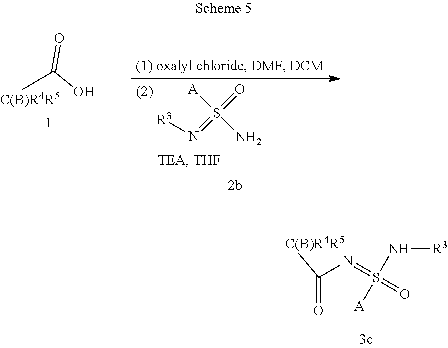

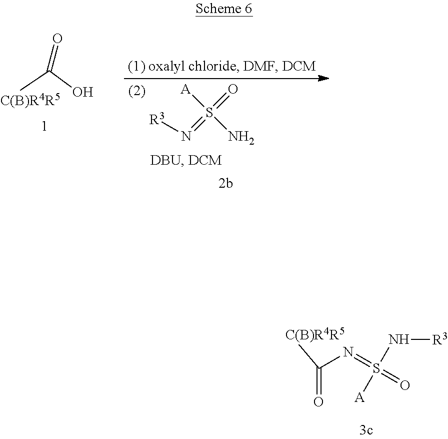

Schemes of Sulfonimidoylamide Intermediates: Schemes 7-12 illustrate the preparation of sulfonimidoylamide intermediates.

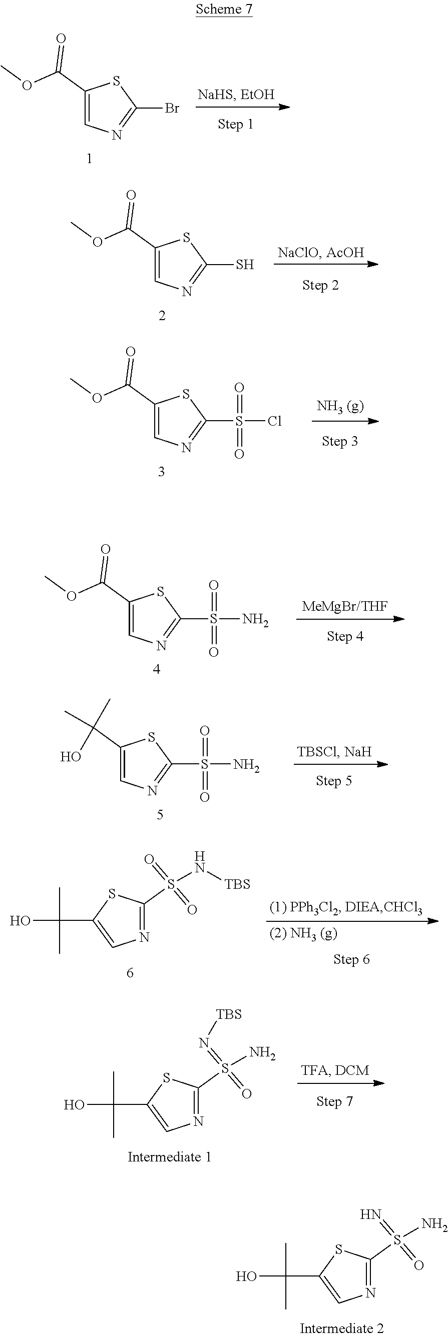

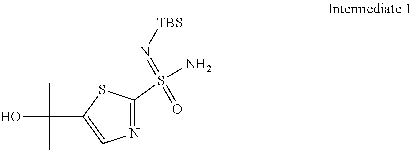

### N′-(tert-butyldimethylsilyl)-5-(2-hydroxypropan-2-yl)thiazole-2-sulfonimidamide

### Step 1: Methyl 2-mercaptothiazole-5-carboxylate

Into a 2000-mL round-bottom flask was placed methyl 2-bromothiazole-5-carboxylate (100 g, 450 mmol), EtOH (1000 mL), and sodium hydrogensulfide (50 g, 890 mmol). The resulting solution was stirred for 2 h at 80° C. and then was cooled to 0° C. with a water/ice bath. The pH value of the solution was adjusted to 3 with hydrogen chloride (1 N). The solids were collected by filtration. This resulted in 63.2 g (80%) of the title compound as a light yellow solid. MS-ESI: 176.0 (M+1).

### Step 2: Methyl 2-(chlorosulfonyl)thiazole-5-carboxylate

Into a 1000-mL round-bottom flask was placed methyl 2-mercaptothiazole-5-carboxylate (30 g, 170 mmol) and acetic acid (300 mL). This was followed by the addition of sodium hypochlorite (300 mL, 8%-10% wt.) in portions at 0° C. The resulting solution was stirred for 2 h at RT and then was diluted with 500 mL of water. The solution was extracted with 3×300 mL of DCM; and the combined organic layers were washed with 2×300 mL of brine, and dried over anhydrous Na₂SO₄. The crude product as a yellow solution in DCM was used in the next step.

### Step 3: Methyl 2-sulfamoylthiazole-5-carboxylate

Into a 2000-mL round-bottom flask was placed methyl 2-(chlorosulfonyl)thiazole-5-carboxylate as a crude solution in DCM (900 mL). To the solution was introduced NH; (g) below 0° C. for 20 minutes. The resulting solution was stirred for 1 h at RT and was then concentrated under vacuum. The residue was applied onto a silica gel column and eluted with ethyl acetate/petroleum ether (1:5 to 1:3). This resulted in 23 g (75%, 2 steps) of the title compound as a white solid. MS-ESI: 223.0 (M+1).

### Step 4: 5-(2-Hydroxypropan-2-yl)thiazole-2-sulfonamide

Into a 500-mL round-bottom flask purged with and maintained under nitrogen was placed a solution of methyl 2-sulfamoylthiazole-5-carboxylate (15 g, 67.5 mmol) in THF (150 mL). This was followed by the addition of MeMgBr/THF (3 M, 90 mL) dropwise with stirring at 0° C. The resulting solution was stirred for 14 h at RT and then was quenched by the addition of 100 mL of NH₄Cl (sat.). The resulting solution was extracted with 3×150 mL of DCM; the organic layers were combined, dried over anhydrous Na₂SO₄, and concentrated under vacuum. The residue was applied onto a silica gel column and eluted with ethyl acetate/petroleum ether (1:5 to 1:3). This resulted in 11.5 g (78%) of the title compound as a white solid. MS-ESI: 223.0 (M+1), 221.0 (M−1) in positive and negative ion mode, respectively.

### Step 5: N-(tert-butyldimethylsilyl)-5-(2-hydroxypropan-2-yl)thiazole-2-sulfonamide

Into a 250-mL 3-necked round-bottom flask purged with and maintained under nitrogen was placed a solution of 5-(2-hydroxypropan-2-yl)thiazole-2-sulfonamide (5 g, 22.5 mmol) in THF (100 mL). Then to the above was added NaH (60% wt, 1.8 g, 45.0 mmol) in portions in an ice/water bath. After stirring for 20 minutes in an ice/water bath, this was followed by the addition of a solution of TBSCl (4.1 g, 27.2 mmol) in THF (10 mL) dropwise with stirring over 2 min at 0° C. The resulting solution was stirred for 4 h at RT. The reaction was quenched with sat. NH₄Cl (100 mL) and extracted with 3×100 mL of ethyl acetate. The combined organic layers were dried over Na₂SO₄ and concentrated under vacuum. The crude solid was washed with ethyl acetate/hexane (1:5) (2×100 mL). This resulted in 6.81 g (90%) of the title compound as a yellow solid. MS-ESI: 337.1 (M+1), 335.1 (M−1) in positive and negative ion mode, respectively.

### Step 6: N′-(tert-butyldimethylsilyl)-5-(2-hydroxypropan-2-yl)thiazole-2-sulfonimidamide

Into a 100-mL 3-necked round-bottom flask purged with and maintained under nitrogen was placed a solution of PPh₃Cl₂ (3 g, 9.0 mmol) in CHCl₃ (100 mL). This was followed by the addition of DIEA (1.54 g, 11.9 mmol) dropwise with stirring at RT. The resulting solution was stirred for 10 min at RT. This was followed by the addition of a solution of N-(tert-butyldimethylsilyl)-5-(2-hydroxypropan-2-yl)thiazole-2-sulfonamide (2.0 g, 5.9 mmol) in CHCl₃ (30 mL) dropwise with stirring in an ice/water bath. The resulting solution was stirred for 30 min in an ice/water bath. To the above was introduced NH₃ (g) below 0° C. for 15 minutes. The resulting solution was stirred for 20 minutes at RT. The solids were filtered out and the filtrate was concentrated and the residue was dissolved in 300 mL of ethyl acetate. The solution was washed with brine (2×100 mL), dried over Na₂SO₄, and concentrated under vacuum. The crude solid was washed with CHCl₃ (100 mL). Then the filtrate was concentrated under vacuum, and the residue was further purified by a silica gel column with ethyl acetate/petroleum ether (1:10 to 1:3). The original washed solid and solid from silica gel purification were combined. This resulted in 1.2 g (60/) of the title compound as a white solid. MS-ESI: 336.1 (M+1). ¹H-NMR (300 MHz, DMSO-d₆) δ 7.66 (s, 1H), 7.12 (s, 2H), 5.78 (s, 1H), 1.51 (s, 6H), 0.86 (s, 9H), 0.02 (s, 3H), 0.01 (s, 3H).

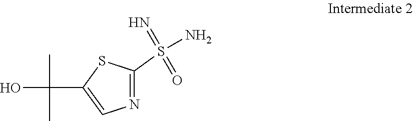

### 5-(2-Hydroxypropan-2-yl)thiazole-2-sulfonimidamide

### Step 7: 5-(2-Hydroxypropan-2-yl)thiazole-2-sulfonimidamide

Into a 50-mL round-bottom flask was placed a solution of N′-(tert-butyldimethylsilyl)-5-(2-hydroxypropan-2-yl)thiazole-2-sulfonimidamide (200 mg, 0.60 mmol), DCM (3 mL), and TFA (0.3 mL). The resulting solution was stirred for 30 min at RT and then was concentrated under 10 vacuum. The crude product was purified by Prep-HPLC using Method E with the following conditions: Column, C18 silica gel, mobile phase, Water (10 mmol/L NH₄HCO₃) and ACN (10% to 50% in 20 min), Detector, UV detection 254/210 nm. This resulted in 100 mg (76%) of the title compound as a light yellow solid. MS-ESI: 222.0 (M+1).

TABLE 2
The Intermediates in the following Table were prepared using similar procedures for
converting compound 6 to Intermediate 2 shown in Scheme 7 by substituting ammonia with
appropriated amine in Step 6.

| | | | Exact Mass |
|---|---|---|---|
| Intermediate # | Structure | IUPAC Name | [M + H]⁺ |
| Intermediate 3 | 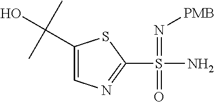 | 5-(2-hydroxypropan-2-yl)-N′-(4- methoxybenzyl)thiazole-2- sulfonimidamide | 342.1 |
| Intermediate 4 | 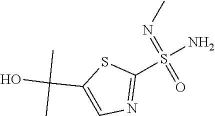 | 5-(2-hydroxypropan-2-yl)-N′- methylthiazole-2-sulfonimidamide | 236.0 |

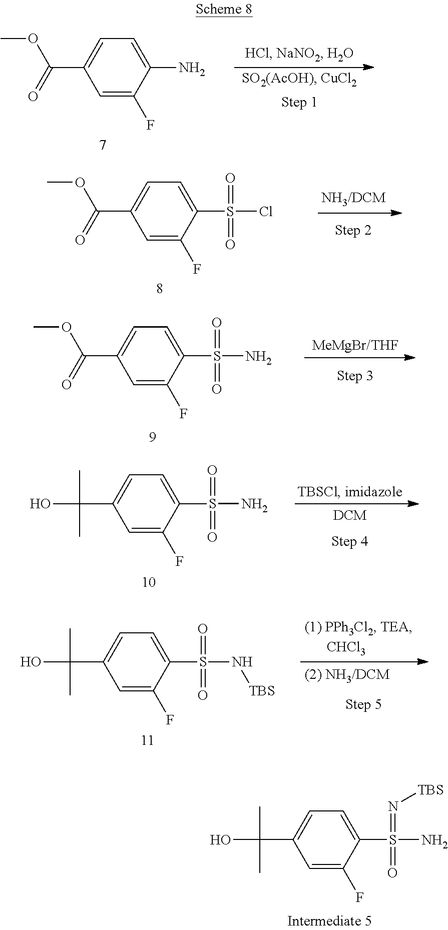

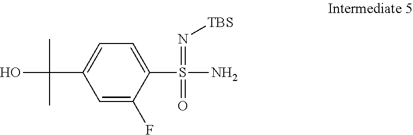

### N′-(tert-butyldimethylsilyl)-2-fluoro-4-(2-hydroxypropan-2-yl)benzenesulfonimidamide

### Step 1: Methyl 4-(chlorosulfonyl)-3-fluorobenzoate

Into a 100-mL round-bottom flask was placed a solution of methyl 4-amino-3-fluorobenzoate (1.0 g, 5.91 mmol) in aq. HCl (6 N, 20 mL). This was followed by the addition of a solution of NaNO₂ (612.4 mg, 8.88 mmol) in water (2 mL) dropwise with stirring at 0° C. The resulting solution was stirred for 30 min at 0° C. The above mixture was added to a saturated solution of SO₂ in AcOH (20 mL) dropwise with stirring at 0° C. Then to the above was added CuCl₂ (0.799 g, 5.96 mmol). The resulting solution was stirred for 1 h at RT and then was quenched by the addition of 20 mL of water. The resulting solution was extracted with 3×20 mL of DCM. The 15 organic layers were combined, dried over anhydrous Na₂SO₄, and then concentrated under vacuum. This resulted in 1 g (crude, 67%) of the title compound as yellow oil. The crude product was used in the next step.

Steps 2-5 used similar procedures for converting compound 3 to Intermediate 1 shown in Scheme 7 to afford Intermediate 5. MS-ESI: 347.2 (M+1).

TABLE 3
The Intermediates in the following Table were prepared using similar procedures for
converting compound 7 to Intermediate 5 shown in Scheme 8 from appropriate
starting materials.

| | | | Exact Mass |
|---|---|---|---|
| Intermediate # | Structure | IUPAC Name | [M + H]⁺ |
| Intermediate 6 | 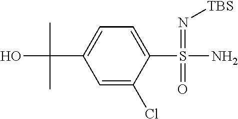 | N′-(tert-butyldimethylsilyl)-2- chloro-4-(2-hydroxypropan-2- yl)benzenesulfonimidamide | 363.1 |
| Intermediate 7 | 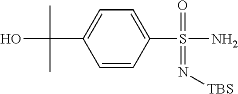 | N′-(tert-butyldimethylsilyl)-4-(2- hydroxypropan-2- yl)benzenesulfonimidamide | 329.1 |

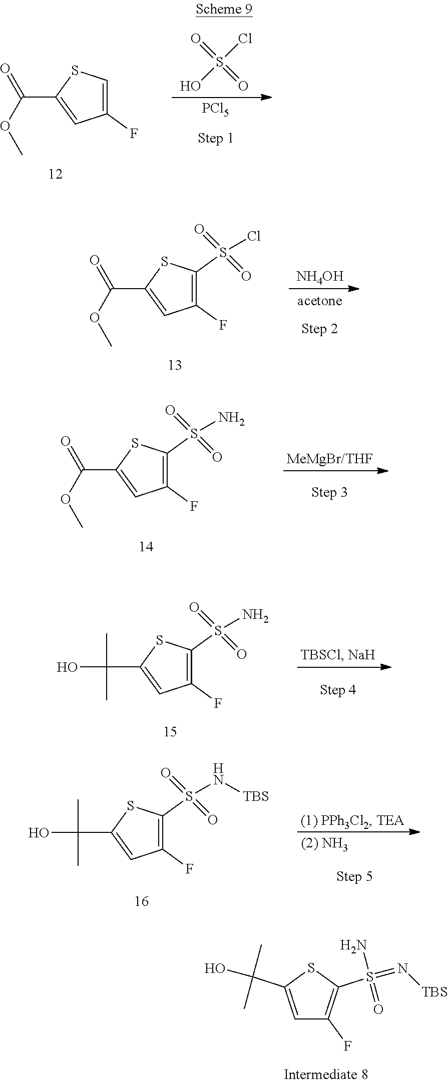

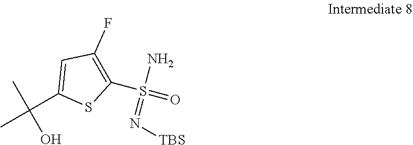

### N′-(tert-butyldimethylsilyl)-3-fluoro-5-(2-hydroxypropan-2-yl)thiophene-2-sulfonimidamide

### Step 1: Methyl 5-(chlorosulfonyl)-4-fluorothiophene-2-carboxylate

Into a 50-mL round-bottom flask was placed a solution of methyl 4-fluorothiophene-2-carboxylate (1.0 g, 6.24 mmol) in CHCl₃ (10 mL). Then to the above was added ClSO₃H (2.18 g, 18.7 mmol). The resulting solution was stirred for 12 h at RT. Then to the above was added PCl₅ (6.5 g, 31.2 mmol). The resulting solution was stirred for 2 h at 50° C. and then was quenched by the addition of 30 mL of water. The resulting solution was extracted with 3×30 mL of ethyl acetate; the organic layers were combined, dried over anhydrous Na₂SO₄, and then concentrated under vacuum. This resulted in 1.2 g (crude, 74%) of the title compound as dark red oil. The crude product was used in the next step.

### Step 2: Methyl 4-fluoro-5-sulfamoylthiophene-2-carboxylate

Into a 50-mL round-bottom flask was placed a solution of methyl 5-(chlorosulfonyl)-4-fluorothiophene-2-carboxylate (600 mg, 2.32 mmol) in acetone (6 mL). Then to the above was added aq. NH₄OH (25% wt., 2 mL). The mixture was stirred for 1 h at RT and then diluted with 10 mL of water. The resulting solution was extracted with 3×10 mL of ethyl acetate; the organic layers were combined, dried over anhydrous Na₂SO₄, and then concentrated under vacuum. This resulted in 500 mg (crude, 90%) of the title compound as yellow oil. MS-ESI: 238.0 (M−1). Step 3-5 used similar procedures for converting compound 4 to Intermediate 1 shown in Scheme 7 to afford Intermediate 8. MS-ESI: 353.1 (M+1).

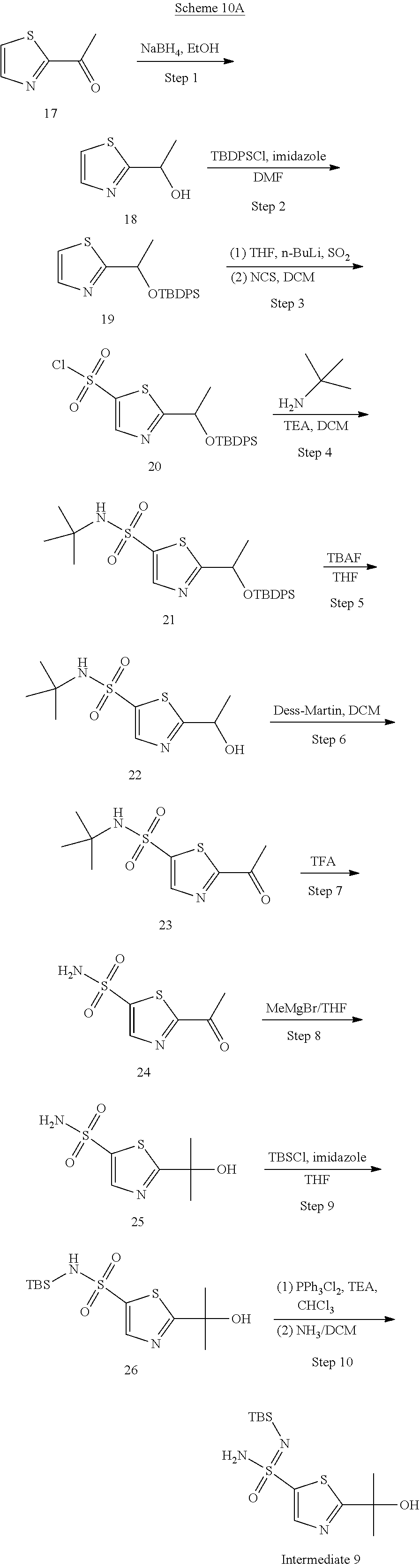

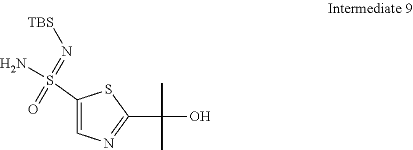

### N′-(tert-butyldimethylsilyl)-2-(2-hydroxypropan-2-yl)thiazole-5-sulfonimidamide

### Step 1: 1-(Thiazol-2-yl)ethanol

Into a 500-mL round-bottom flask was placed 1-(thiazol-2-yl)ethanone (20 g, 157 mmol) in EtOH (200 mL). This was followed by the addition of NaBH₄ (3 g, 81.3 mmol) in portions at 0° C. The resulting solution was stirred for 2 h at RT and was then quenched by the addition of 10 mL of NH₄Cl (sat.). The resulting solution was diluted with 200 mL of water and extracted with 2×200 mL of DCM. The organic layers were combined, dried over anhydrous Na₂SO₄, and then concentrated under vacuum. This resulted in 20 g (98%) of the title compound as light yellow oil. MS-ESI: 130.0 (M+1).

### Step 2: 2-(1-(Tert-butyldiphenylsilyloxy)ethyl)thiazole

Into a 500-mL round-bottom flask was placed 1-(thiazol-2-yl)ethanol (20 g, 154.8 mmol), DMF (150 mL), and imidazole (20.5 g, 301 mmol). This was followed by the addition of TBDPSCl (46 g, 167 mmol) dropwise with stirring at 0° C. The mixture was stirred for 2 h at RT and then was diluted with 300 mL of water. The resulting solution was extracted with 3×200 mL of DCM. The organic layers were combined and concentrated under vacuum. The residue was applied onto a silica gel column and eluted with ethyl acetate/petroleum ether (1:100 to 1:80). This resulted in 55 g (97%) of the title compound as a colorless oil. MS-ESI: 368.1 (M+1).

### Step 3: 2-(1-(Tert-butyldiphenylsilyloxy)ethyl)thiazole-5-sulfonyl chloride

Into a 500-mL 3-necked round-bottom flask purged with and maintained under nitrogen was placed 2-(1-(tert-butyldiphenylsilyloxy)ethyl)thiazole (30 g, 81.6 mmol) and THF (200 mL). This was followed by the addition of n-BuLi/THF (2.5 M, 35.2 mL) dropwise with stirring at −78° C. The resulting solution was stirred for 0.5 h at −78° C., and then SO₂ was introduced into the above reaction mixture. The reaction was slowly warmed to RT, and then NCS (12.8 g, 95.86 mmol) was added. The resulting solution was stirred for 1 h at RT. The solids were filtered out. The resulting filtrate was concentrated under vacuum. This resulted in 30 g (crude, 79%) of the title compound as brown oil. The crude product was used in the next step directly.

### Step 4: N-tert-butyl-2-(1-(tert-butyldiphenylsilyloxy)ethyl)thiazole-5-sulfonamide

Into a 500-mL round-bottom flask was placed 2-(1-(tert-butyldiphenylsilyloxy)ethyl)thiazole-5-sulfonyl chloride (crude, 30 g, 64.4 mmol), DCM (200 mL), and TEA (13 g, 128 mmol). This was followed by the addition of 2-methylpropan-2-amine (5.6 g, 76.6 mmol) dropwise with stirring at 0° C. The resulting solution was stirred for 2 h at RT and then was concentrated under vacuum. The residue was applied onto a silica gel column and eluted with ethyl acetate/petroleum ether (1:30 to 1:20). This resulted in 25 g (77%) of the title compound as brown oil. MS-ESI: 503.2 (M+1).

### Step 5: N-tert-butyl-2-(1-hydroxyethyl)thiazole-5-sulfonamide

Into a 500-mL round-bottom flask was placed N-tert-butyl-2-(1-(tert-butyldiphenylsilyloxy)ethyl)thiazole-5-sulfonamide (25 g, 49.7 mmol), THF (200 mL), and TBAF (30 g, 99.67 mmol). The resulting solution was stirred for 2 h at RT and then was diluted with 200 mL of water. The resulting solution was extracted with 3×200 mL of DCM. The organic layers were combined and concentrated under vacuum. The residue was applied onto a silica gel column and eluted with ethyl acetate/petroleum ether (1:20 to 1:10). This resulted in 12 g (91%) of the title compound as light yellow oil. MS-ESI: 265.1 (M+1).

### Step 6: 2-Acetyl-N-tert-butylthiazole-5-sulfonamide

Into a 500-mL round-bottom flask was placed a solution of N-tert-butyl-2-(1-hydroxyethyl)thiazole-5-sulfonamide (12 g, 45.4 mmol) in DCM (200 mL). To this solution was added Dess-Martin reagent (20 g, 47.2 mmol) in portions at RT. The resulting solution was stirred for 2 h at RT and then was concentrated under vacuum. The residue was applied onto a silica gel column and eluted with ethyl acetate/petroleum ether (1:20 to 1:10). This resulted in 9 g (76%) of the title compound as a light yellow solid. MS-ESI: 263.0 (M+1).

### Step 7: 2-Acetylthiazole-5-sulfonamide

Into a 100-mL round-bottom flask was placed a solution of 2-acetyl-N-tert-butylthiazole-5-sulfonamide (7 g, 26.7 mmol) in DCM (20 mL). To the solution was added TFA (20 mL) at RT. The resulting solution was stirred for 14 h at 70° C. and then was concentrated under vacuum. The residue was applied onto a silica gel column and eluted with ethyl acetate/petroleum ether (1:5 to 1:3). This resulted in 5 g (91%) of the title compound as a yellow solid. MS-ESI: 207.0 (M+1).

### Step 8: 2-(2-Hydroxypropan-2-yl)thiazole-5-sulfonamide

Into a 500-mL 3-necked round-bottom flask purged with and maintained under nitrogen was placed 2-acetylthiazole-5-sulfonamide (5 g, 4.85 mmol) in THF (100 mL). This was followed by the addition of MeMgBr (3 M in THF, 8.1 mL, 24.3 mmol) dropwise with stirring at 0° C. The resulting solution was stirred for 14 h at RT and then was quenched by the addition of 100 mL of NH₄Cl (sat.). The resulting solution was extracted with 2×150 mL of DCM. The organic layers were combined and concentrated under vacuum. The residue was applied onto a silica gel column and eluted with ethyl acetate/petroleum ether (1:5 to 1:3). This resulted in 2.9 g (54%) of the title compound as a light yellow solid. MS-ESI: 223.0 (M+1).

### Step 9: N-(tert-butyldimethylsilyl)-2-(2-hydroxypropan-2-yl)thiazole-5-sulfonamide

Into a 100-mL 3-necked round-bottom flask purged with and maintained under nitrogen was placed a solution of 2-(2-hydroxypropan-2-yl)thiazole-5-sulfonamide (1.5 g, 6.75 mmol) in THF (20 mL). Then to the above was added imidazole (0.92 g, 13.5 mmol). This was followed by the addition of a solution of TBSCl (5.1 g, 34 mmol) in THF (5 mL) dropwise with stirring over 2 min at 0° C. The resulting solution was stirred for 16 h at RT and then was concentrated under vacuum. The residue was applied onto a silica gel column and eluted with ethyl acetate/petroleum ether (1:3 to 1:2). This resulted in 1.13 g (50%/) of the title compound as a yellow solid. MS-ESI: 337.1 (M+1).

Steps 10 used similar procedures employed for converting compound 6 to Intermediate 1 shown in Scheme 7 to afford Intermediate 9. MS-ESI: 336.1 (M+1).

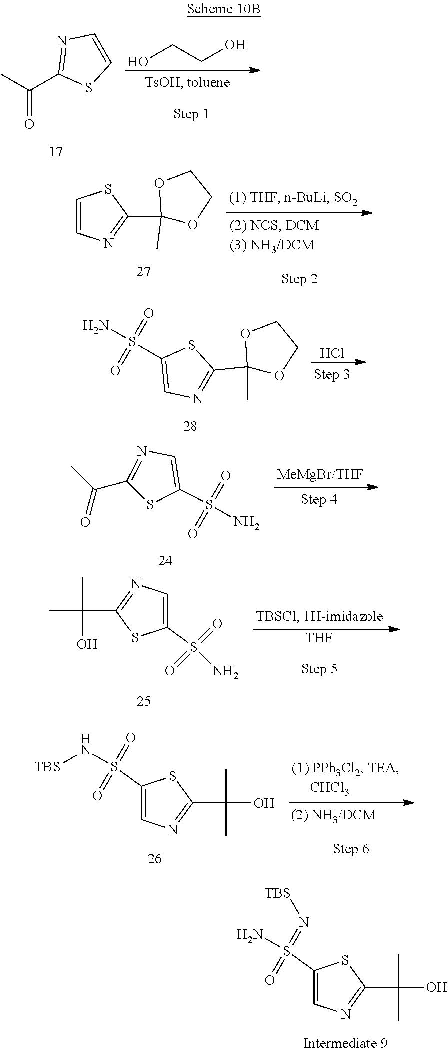

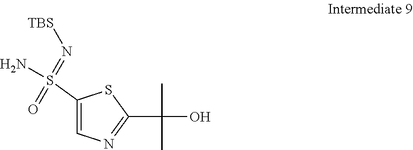

### N′-(tert-butyldimethylsilyl)-2-(2-hydroxypropan-2-yl)thiazole-5-sulfonimidamide

### Step 1: 2-(2-Methyl-1,3-dioaolan-2-yl)thiazole

Into a 500-mL round-bottom flask was placed a solution of 1-(thiazol-2-yl)ethanone (20 g, 157 mmol) in toluene (300 mL). To the solution was added TsOH (2.7 g, 15.7 mmol)) and ethane-1,2-diol (19.5 g, 314 mmol). The resulting solution was refluxed overnight, and water was separated from the solution during refluxing. The resulting solution was diluted with 200 mL of water and extracted with 2×100 mL of ethyl acetate. The organic layers were combined and dried over anhydrous Na₂SO₄, then concentrated under vacuum. This resulted in 26.6 g (99%) of the title compound as light yellow oil. MS-ESI: 172.0 (M+1).

### Step 2: 2-(2-Methyl-1,3-dioxolan-2-yl)thiazole-5-sulfonamide

Into a 500-mL 3-necked round-bottom flask purged with and maintained under nitrogen was placed a solution of 2-(2-methyl-1,3-dioxolan-2-yl)thiazole (14 g, 81.6 mmol) in THF (200 mL). This was followed by the addition of n-BuLi (2.5 M in THF, 35.2 mL) dropwise with stirring at −78° C. The resulting solution was stirred for 0.5 h at −78° C. and then SO₂ gas was introduced into the above reaction mixture. The reaction was slowly warmed to RT and then NCS (12.8 g, 95.86 mmol) was added. The resulting solution was stirred for 1 h at RT. The solids were filtered out. The resulting filtrate was concentrated under vacuum and was then diluted with DCM (160 mL). To the above was added a saturated solution of ammonia in DCM (300 mL). The resulting solution was stirred for 3 h at RT and then was concentrated under vacuum. The residue was applied onto a silica gel column and eluted with a gradient of ethyl acetate/petroleum ether (1:20 to 1:5). This resulted in 12.5 g (61%) of the title compound as a yellow solid. MS-ESI: 251.0 (M+1).

### Step 3: 2-Acetylthiazole-5-sulfonamide

Into a 250-mL round-bottom flask was placed a solution of 2-(2-methyl-1,3-dioxolan-2-yl)thiazole-5-sulfonamide (12.5 g, 50 mmol) in THF (125 mL). To the above was added aq. HCl (4 N, 50 mL). The resulting solution was stirred for 6 h at 70° C. The resulting solution was diluted with 100 mL of water and extracted with 2×200 mL of ethyl acetate. The organic layers were combined, dried over anhydrous Na₂SO₄, and then concentrated under vacuum. The residue was applied onto a silica gel column and eluted with a gradient of ethyl acetate/petroleum ether (1:2 to 1:1). This resulted in 9.3 g (90%) of the title compound as a yellow solid. MS-ESI: 207.0 (M+1).

Steps 4-6 used the same procedures for converting compound 24 to Intermediate 9 shown in Scheme 10A to afford Intermediate 9. MS-ESI: 336.1 (M+1).

TABLE 4
The Intermediates in the following Table were prepared using the similar procedures for converting
compound 17 to Intermediate 9 shown in Scheme 10B from appropriate starting materials.

| | | | Exact Mass |
|---|---|---|---|
| Intermediate # | Structure | IUPAC Name | [M + H]⁺ |
| Intermediate 10 | 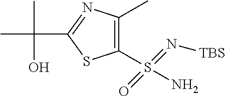 | N′-(tert-butyldimethylsilyl)-2-(2- hydroxypropan-2-yl)-4- methylthiazole-5-sulfonimidamide | 350.2 |

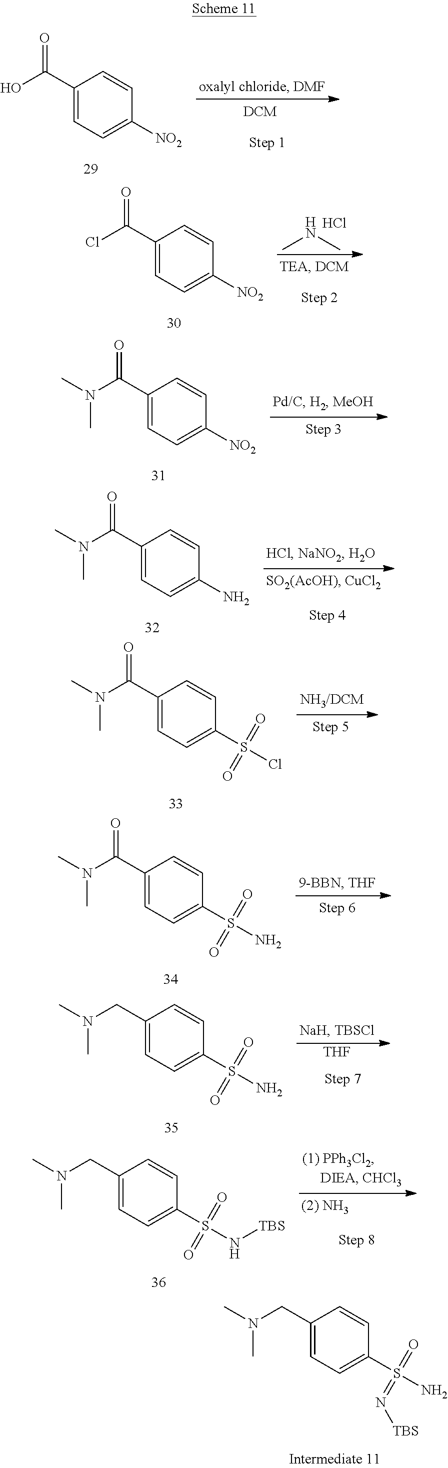

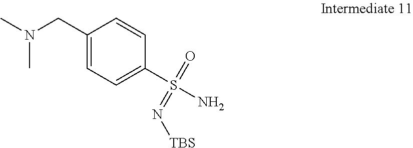

### N′-(tert-butyldimethylsilyl)-4-((dimethylamino)methyl)benzenesulfonimidamide

### Step 1: 4-Nitrobenzoyl Chloride

Into a 500-mL round-bottom flask was placed 4-nitrobenzoic acid (20 g, 120 mmol), DCM (200 mL), and DMF (0.2 mL). This was followed by the addition of oxalyl chloride (15 mL, 135 mmol) dropwise with stirring at 0° C. The resulting solution was stirred for 4 h at RT and then was concentrated under vacuum. This resulted in 22 g (crude) of the title compound as yellow oil. The crude product was used in the next step.

### Step 2: N,N-dimethyl-4-nitrobenzamide

Into a 500-mL round-bottom flask was placed dimethylamine hydrochloride (9.8 g, 120 mmol), DCM (200 mL), and TEA (41.5 mL, 300 mmol). This was followed by the addition of 4-nitrobenzoyl chloride (22 g, crude) dropwise with stirring at 0° C. The resulting solution was stirred for 6 h at RT and then was concentrated under vacuum. The resulting mixture was washed with 2×50 mL of water. The solids were collected by filtration. This resulted in 16 g (69%, 2 steps) of the title compound as a white solid. MS-ESI: 195.1 (M+1).

### Step 3: 4-Amino-N,N-dimethylbenzamide

Into a 250-mL round-bottom flask was placed N,N-dimethyl-4-nitrobenzamide (16 g, 82.4 mmol), MeOH (100 mL), and Pd/C (10% wt., 1 g). The flask was evacuated and flushed three times with hydrogen. The resulting solution was stirred for 12 h at RT under an atmosphere of hydrogen. The Pd/C catalysts were filtered out, and the filtrate was concentrated under vacuum. This resulted in 13 g (96%) of the title compound as a white solid. MS-ESI: 165.1 (M+1).

### Step 4: 4-(Dimethylcarbamoyl)benzene-1-sulfonyl chloride

Into a 50-mL round-bottom flask was placed 4-amino-N,N-dimethylbenzamide (3 g, 18.3 mmol) and HCl (6 M, 12 mL). This was followed by the addition of a solution of NaNO₂ (1.5 g, 21.7 mmol) in water (3 mL) dropwise with stirring at 0° C. The resulting solution was stirred for 30 min at 0° C. The above mixture was added to a saturated solution of SO₂ in AcOH (100 mL) dropwise with stirring at 0° C. To the above was added CuCl₂ (4.8 g, 35.7 mmol). The resulting solution was stirred for 2 h at RT and then was quenched by the addition of 100 mL of water. The resulting solution was extracted with 2×100 mL of DCM. The organic layers were combined, dried over anhydrous Na₂SO₄, and then concentrated under vacuum. This resulted in 5 g (crude) of the title compound as yellow oil. The crude product was used in the next step.

### Step 5: N,N-dimethyl-4-sulfamoylbenzamide

Into a 250-mL round-bottom flask was placed 4-(dimethylcarbamoyl)benzene-1-sulfonyl chloride (5 g, 20.2 mmol) in DCM (20 mL). To the above was added a saturated solution of ammonia in DCM (80 mL). The resulting solution was stirred for 2 h at RT and then was concentrated under vacuum. The resulting mixture was washed with 3×100 mL of ethyl acetate. The solids were filtered out. The resulting filtrate was concentrated under vacuum. This resulted in 3.1 g (67%) of the title compound as a white solid. MS-ESI: 229.1 (M+1).

### Step 6: 4-((Dimethylamino)methyl)benzenesulfonamide

Into a 100-mL round-bottom flask purged with and maintained under nitrogen was placed a solution of N,N-dimethyl-4-sulfamoylbenzamide (1.8 g, 7.9 mmol) in THF (50 mL). This was followed by the addition of 9-BBN (5.8 g, 47.5 mmol) in portions at 0° C. The resulting solution was stirred for 12 h at 70° C. and then was quenched by the addition of 20 mL of water/ice. The resulting solution was extracted with 3×100 mL of ethyl acetate and the organic layers were combined. The resulting mixture was washed with 200 mL of water and then the organic layer was concentrated under vacuum. The residue was applied onto a silica gel column and eluted with DCM/MeOH (20:1 to 15:1). This resulted in 1 g (59%) of the title compound as a white solid. MS-ESI: 215.1 (M+1).

### Step 7: N-(tert-butyldimethylsilyl)-4-((dimethylamino)methyl)benzenesulfonamide

Into a 250-mL round-bottom flask was placed a solution of 4-((dimethylamino)methyl)benzenesulfonamide (500 mg, 2.33 mmol) in THF (40 mL). This was followed by the addition NaH (60% wt., 170 mg) in portions at 0° C. Then TBSCl (1.75 g, 11.6 mmol) was added. The resulting solution was stirred for 2 h at RT and then was quenched by the addition of 30 mL of water. The resulting solution was extracted with 3×40 mL of ethyl acetate and the organic layers combined and concentrated under vacuum. The residue was applied onto a silica gel column and eluted with DCM/MeOH (30:1 to 20:1). This resulted in 540 mg (70%) of the title compound as a yellow solid. MS-ESI: 329.2 (M+1).

Step 8 used similar procedures for converting compound 6 to Intermediate 1 shown in Scheme 7 to afford Intermediate 11. MS-ESI: 328.2 (M+1).

TABLE 4
The Intermediates in the following Table were prepared using the similar procedures for
converting compound 29 to Intermediate 11 shown in Scheme 11 from appropriate starting materials.

| | | | Exact Mass |
|---|---|---|---|
| Intermediate # | Structure | IUPAC Name | [M + H]⁺ |
| Intermediate 12 | 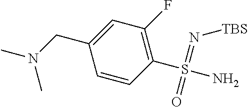 | N′-(tert-butyldimethylsilyl)-4- ((dimethylamino)methyl)-2- fluorobenzenesulfonimidamide | 346.2 |

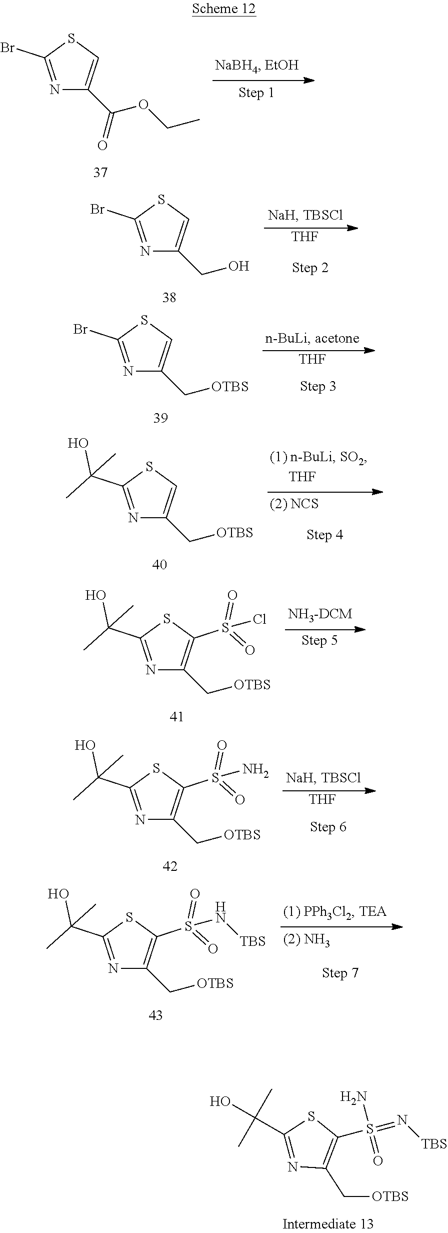

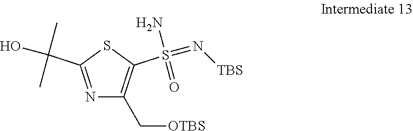

### N′-(tert-butyldimethylsilyl)-4-((tert-butyldimethylsilyloxy)methyl)-2-(2-hydroxypropan-2-yl)thiazole-5-sulfonimidamide

### Step 1: (2-Bromothiazol-4-yl)methanol

Into a 100-mL round-bottom flask was placed a solution of ethyl 2-bromo-1,3-thiazole-4-carboxylate (3 g, 12.71 mmol) in EtOH (30 mL). NaBH₄ (1.0 g, 25.41 mmol) was added in portions with an ice/water bath. The resulting solution was stirred for 3 hr at room temperature. The reaction was then quenched by the addition of 100 mL of water in an ice/water bath. The resulting solution was extracted with 3×100 ml of ethyl acetate, and the combined organic layers were concentrated. This resulted in 2 g (81%) of the title compound as yellow oil. MS-ESI: 196.2, 194.2 (M+1).

### Step 2: 2-Bromo-4-((tert-butyldimethylsilyloxy)methyl)thiazole

Into a 100-mL round-bottom flask was placed a solution of (2-bromo-1,3-thiazol-4-yl)methanol (2.0 g, 10.31 mmol) in THF (20 mL). To the solution was added NaH (60% wt., 1.2 g, 30.92 mmol) in portions with an ice/water bath. After stirring for 15 minutes at RT, a solution of TBSCl (4.7 g, 30.92 mmol) in THF (5 mL) was added dropwise in an ice/water bath. The resulting solution was stirred for 2 hr at RT. The reaction was then quenched by the addition of 50 mL of water. The resulting solution was extracted with 3×100 ml of ethyl acetate, the organic layers were combined, dried over anhydrous Na₂SO₄ and concentrated under vacuum. The residue was applied onto a silica gel column and eluted with ethyl acetate/petroleum ether (1:30). This resulted in 2.5 g (79%) of the title compound as yellow oil. MS-ESI: 310.2, 308.2 (M+1).

### Step 3: 2-(4-((Tert-butyldimethylsilyloxy)methyl)thiazol-2-yl)propan-2-ol

Into a 100-mL round-bottom flask purged and maintained with an inert atmosphere of nitrogen was placed a solution of 2-bromo-4-[[(tert-butyldimethylsilyl)oxy]methyl]-1,3-thiazole (2.5 g, 8.11 mmol) in THF (30 mL). To this solution was added n-BuLi (2.5 M in hexane, 4.86 mL, 12.16 mmol) dropwise at −78° C.; and the resulting mixture was stirred for 30 min at −78° C. To the above was added acetone (0.9 g, 16.22 mmol) dropwise at −78° C. The ensuing solution was then stirred for 1 hr at RT, after which the reaction was quenched by the addition of 100 mL of water. The resulting solution was extracted with 3×100 ml of ethyl acetate; the organic layers were combined, dried over anhydrous Na₂SO₄, and concentrated under vacuum. The residue was applied onto a silica gel column and eluted with ethyl acetate/petroleum ether (1:10). This resulted in 2 g (86%) of the title compound as yellow oil. MS-ESI: 288.2 (M+1).

### Step 4: 4-((Tert-butyldimethylsilyloxy)methyl)-2-(2-hydroxypropan-2-yl)thiazole-5-sulfonyl chloride

Into a 250-mL round-bottom flask purged and maintained with an inert atmosphere of nitrogen was placed a solution of 2-(4-[[(tert-butyldimethylsilyl)oxy]methyl]-1,3-thiazol-2-yl)propan-2-ol (2 g, 6.96 mmol) in THF (20 mL). To this solution was added n-BuLi (2.5 M in hexane, 8.4 mL, 20.9 mmol) dropwise at −78° C.; the mixture was stirred for 30 min at −78° C. Then SO₂ was introduced in this solution for 10 minutes below −30° C. and stirred for 30 min at RT. The resulting solution was concentrated under vacuum. The crude solid was dissolved in DCM (30 ml), followed by the addition of NCS (1.4 g, 10.4 mmol) in portions in an ice/water bath. The solution was stirred for 2 hr at RT. The resulting mixture was concentrated under vacuum. This resulted in 2.5 g (crude) of the title compound as a yellow solid.

### Step 5: 4-((Tert-butyldimethylsilyloxy)methyl)-2-(2-hydroxypropan-2-yl)thiazole-5-sulfonamide

Into a 100-mL round-bottom flask was placed a solution of 4-[[(tert-butyldimethylsilyl)oxy]methyl]-2-(2-hydroxypropan-2-yl)-1,3-thiazole-5-sulfonyl chloride (2.5 g, 6.48 mmol) in DCM (30 mL). To the above was added a saturated solution of ammonia in DCM (10 mL) in an ice/water bath. The resulting solution was stirred for 1 hr at room temperature. The resulting mixture was concentrated. The residue was applied onto a silica gel column and eluted with ethyl acetate/petroleum ether (1:5). This resulted in 1.2 g (51%) of the title compound as yellow oil. MS-ESI: 367.2 (M+1).

### Step 6: N-(tert-butyldimethylsilyl)-4-((tert-butyldimethylsilyloxy)methyl)-2-(2-hydroxypropan-2-yl)thiazole-5-sulfonamide

To a solution of 2-(2-hydroxypropan-2-yl)-1,3-thiazole-5-sulfonamide (1.2 g, 3.27 mmol) in THF (20 mL), NaH (60% wt., 0.4 g, 9.82 mmol) was added in portions with an ice/water bath. After stirring for 15 minutes at RT, a solution of TBSCl (1.5 g, 9.82 mmol) in THF (5 mL) was added dropwise in an ice/water bath. The resulting solution was stirred for 2 hr at RT. The reaction was quenched by the addition of 100 mL of water. The resulting solution was extracted with 3×100 ml of ethyl acetate, the organic layers were combined, dried over anhydrous Na₂SO₄ and concentrated under vacuum. This resulted in 1.3 g (83%) of the title compound as yellow oil. MS-ESI: 481.2 (M+1).

### Step 7: N′-(tert-butyldimethylsilyl)-4-((tert-butyldimethylsilyloxy)methyl)-2-(2-hydroxypropan-2-yl)thiazole-5-sulfonimidamide

Into a 100-mL round-bottom flask purged and maintained with an inert atmosphere of nitrogen was placed a solution of PPh₃Cl₂ (1.4 g, 4.06 mmol) in CHCl₃ (10 mL), TEA (0.8 g, 8.11 mmol) was added dropwise in an ice/water bath. The solution was stirred at RT for 20 minutes. To this solution was added N-(tert-butyldimethylsilyl)-4-[[(tert-butyldimethylsilyl)oxy]methyl]-2-(2-hydroxypropan-2-yl)-1,3-thiazole-5-sulfonamide (1.3 g, 2.70 mmol) in CHCl₃ (10 mL) dropwise in ice/water bath, the solution was stirred for 0.5 hr at RT. A saturated solution of ammonia in DCM (20 mL) was poured into this solution at 0° C. The solution was stirred for 1 hr at RT. The resulting solution was concentrated under vacuum. The residue was applied onto a silica gel column and eluted with ethyl acetate/petroleum ether (1:5). This resulted in 600 mg (46%) of the title compound as a yellow solid. MS-ESI: 480.2 (M+1).

Schemes for phenylacetic acids Intermediates: Schemes 13-22 illustrate the preparation of phenylacetic acid intermediates.

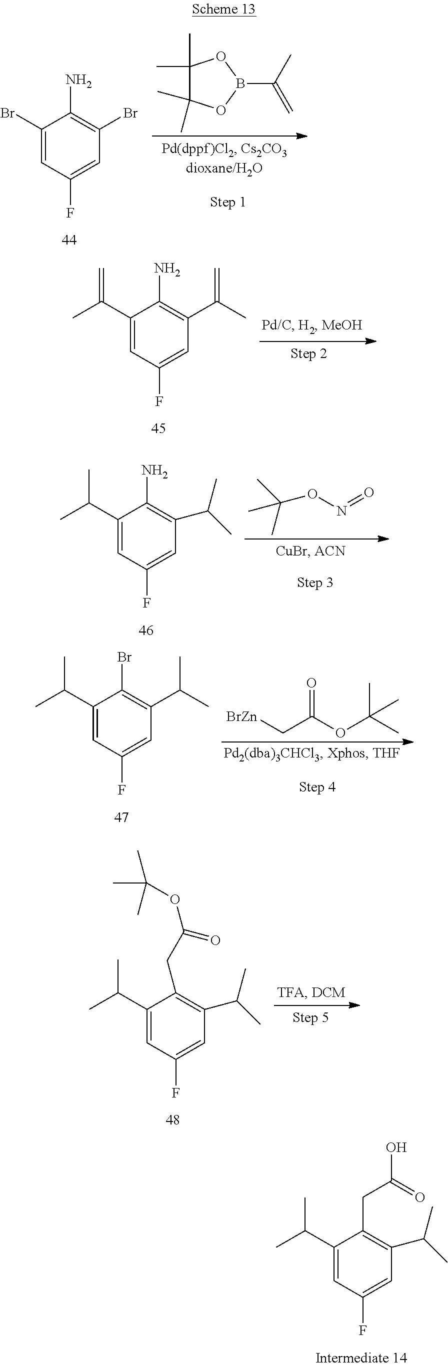

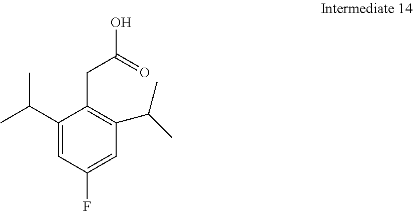 [SMILES: CC(C)c1cc(F)cc(C(C)C)c1CC(=O)O]

### 2-(4-Fluoro-2,6-diisopropylphenyl)acetic acid

### Step 1: 4-Fluoro-2,6-di(prop-1-en-2-yl)aniline

Into a 500-mL round-bottom flask purged with and maintained under nitrogen was placed 2,6-dibromo-4-fluoroaniline (15 g, 55.8 mmol), dioxane (150 mL), water (15 mL), Cs₂CO₃ (55 g, 169 mmol), 4,4,5,5-tetramethyl-2-(prop-1-en-2-yl)-1,3,2-dioxaborolane (25 g, 149 mmol), and Pd(dppf)Cl₂ (4 g, 5.47 mmol). The resulting solution was stirred for 15 h at 100° C. and then was concentrated under vacuum. The mixture was diluted with 300 mL water, and extracted with ethyl acetate (3×300 mL). The organic layers were combined, dried over anhydrous Na₂SO₄, and concentrated under vacuum. The residue was applied onto a silica gel column and eluted with ethyl acetate/petroleum ether (1:10 to 1:8). This resulted in 9.2 g (86%) of the title compound as brown oil. MS-ESI: 192.1 (M+1).

### Step 2: 4-Fluoro-2,6-bis(propan-2-yl)aniline

Into a 500-mL round-bottom flask was placed 4-fluoro-2,6-bis(prop-1-en-2-yl)aniline (9.2 g, 48.1 mmol) in MeOH (200 mL). Then Pd/C (10% wt, 900 mg) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:10 to 1:8). This resulted in 7.2 g (77%) of the title compound as brown oil. MS-ESI: 196.1 (M+1).

### Step 3: 2-Bromo-5-fluoro-1,3-bis(propan-2-yl)benzene

Into a 500-mL round-bottom flask purged with and maintained under nitrogen was placed 4-fluoro-2,6-bis(propan-2-yl)aniline (7 g, 35.9 mmol), ACN (300 mL), and CuBr (7.71 g, 53.9 mmol). This was followed by the addition of tert-butyl nitrite (5.55 g, 53.8 mmol) dropwise with stirring at 0° C. The resulting solution was stirred for 3 h at 60° C. and then was concentrated under vacuum. The residue was applied onto a silica gel column eluted with petroleum ether. This resulted in 3.0 g (32%) of the title compound as yellow oil. ¹H NMR (400 MHz, DMSO-d₆): δ 7.09 (d, J=9.8 Hz, 2H), 3.40 (hept, J=6.9 Hz, 2H), 1.20 (d, J=6.8 Hz, 12H).

### Step 4: Tert-butyl 2-[4-fluoro-2,6-bis(propan-2-yl)phenyl]acetate

Into a 250-mL 3-necked round-bottom flask purged with and maintained under nitrogen was placed 2-bromo-5-fluoro-1,3-bis(propan-2-yl)benzene (3.0 g, 11.6 mmol), THF (150 mL), X-phos (553 mg, 1.16 mmol), and Pd₂(dba)CHCl₃ (600 mg, 0.58 mmol). The resulting solution was stirred for 0.5 h at RT. Then, to the above, tert-butyl 2-(bromozincio)acetate (6.0 g, 23.04 mmol) was added. The resulting solution was stirred for 5 h at 70° C., after which it was quenched by the addition of 100 mL of NH₄Cl (sat.). The resulting solution was extracted with 3×100 mL of ethyl acetate, and the organic layers combined and concentrated under vacuum. The residue was applied onto a silica gel column and eluted with ethyl acetate/petroleum ether (1:100 to 3:97). This resulted in 3.14 g (92%) of the title compound as yellow oil. ¹H NMR (400 MHz, DMSO-d₆) δ 6.93 (d, J=10.4 Hz, 2H), 3.67 (s, 2H), 3.19-3.07 (m, 2H), 1.39 (s, 9H), 1.15 (d, J=6.7 Hz, 12H).

### Step 5: 2-(4-Fluoro-2,6-diisopropylphenyl)acetic acid

Into a 50-mL round-bottom flask was placed tert-butyl 2-[4-fluoro-2,6-bis(propan-2-yl)phenyl]acetate (1.56 g, 5.30 mmol), DCM (10 mL), and TFA (10 mL). The resulting solution was stirred for 3 h at RT and was then concentrated under vacuum. The crude product was dissolved in 100 mL of NaOH (4 N) and washed with 3×50 mL of DCM to remove impurities. The pH value of aqueous phase was adjusted to 2 with HCl (4 N); the aqueous phase was then extracted with 3×100 mL of DCM. The organic layers were combined, dried over anhydrous Na₂SO₄, and concentrated under vacuum. This resulted in 1.09 g (86%) of the title compound as a light yellow solid. MS-ESI: 237.1 (M−1).

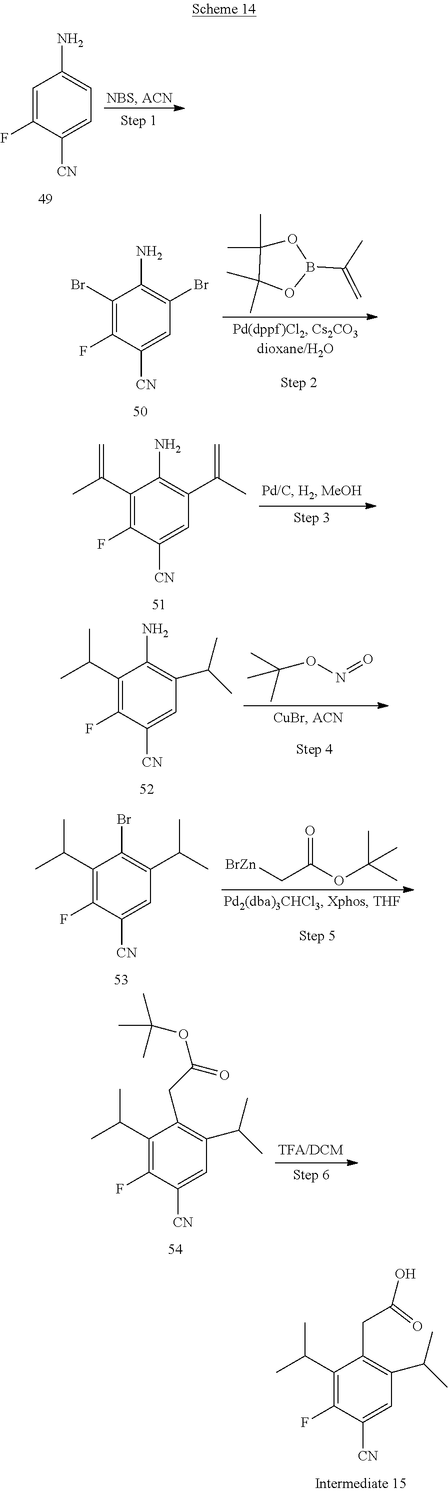

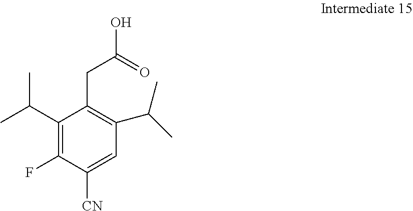 [SMILES: CC(C)c1cc(C#N)c(F)c(C(C)C)c1CC(=O)O]

### 2-(4-Cyano-3-fluoro-2,6-diisooropylphenyl)acetic acid

### Step 1: 4-amino-3,5-dibromo-2-fluorobenzonitrile

Into a 1000-mL round-bottom flask was placed 4-amino-2-fluorobenzonitrile (25 g, 184 mmol), ACN (500 mL), and NBS (81.7 g, 459 mmol). The resulting solution was stirred overnight at 75′C and then was concentrated under vacuum. The residue was applied onto a silica gel column and eluted with ethyl acetate/petroleum ether (1:100 to 1:98). This resulted in 50 g (93%) of the title compound as brown oil. MS-ESI: 294.9/292.9/296.9 (M+1).

Steps 2-6 used similar procedures for converting compound 44 to Intermediate 14 shown in Scheme 13 to afford Intermediate 15. MS-ESI: 262.1 (M−1).

TABLE 5
The Intermediate in the following Table was prepared using the similar procedures for
converting compound 49 to Intermediate 15 shown in Scheme 14 from appropriated
starting materials.

| | | | Exact Mass |
|---|---|---|---|
| Intermediate # | Structure | IUPAC Name | [M − H]⁺ |
| Intermediate 16 | 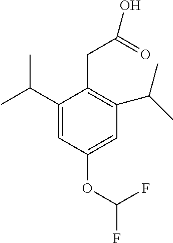 [SMILES: CC(C)c1cc(OC(F)F)cc(C(C)C)c1CC(=O)O] | 2-(4-(Difluoromethoxy)-2,6- diisopropylphenyl)acetic acid | 285.1 |

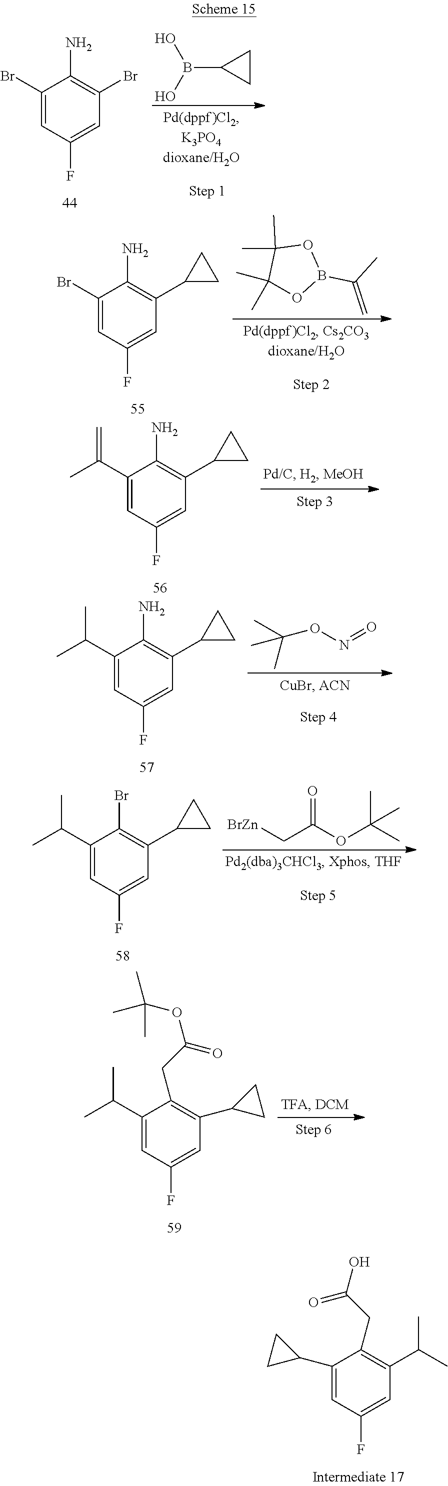

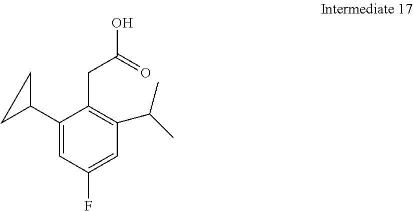 [SMILES: CC(C)c1cc(F)cc(C2CC2)c1CC(=O)O]

### 2-(2-Cyclopropyl-4-fluoro-6-isopropylphenyl)acetic acid

### Step 1: 2-Bromo-6-cyclopropyl-4-fluorobenzenamine

Into a 500-mL round-bottom flask purged with and maintained under nitrogen was placed 2,6-dibromo-4-fluorobenzenamine (10 g, 37.2 mmol), 1,4-dioxane (200 mL), water (10 mL), K₃PO₄ (23.6 g, 111 mmol), cyclopropylboronic acid (9.59 g, 112 mmol), and Pd(dppf)Cl₂ (1.36 g, 1.86 mmol). The resulting solution was stirred overnight at 90° C. and then was concentrated under vacuum. The residue was applied onto a silica gel column and eluted with ethyl acetate/petroleum ether (1:40 to 1:20). This resulted in 3.4 g (40%) of the title compound as light yellow oil. MS-ESI: 230.0 (M+1).

### Step 2: 2-Cyclopropyl-4-fluoro-6-(prop-1-en-2-yl)benzenamine

Into a 250-mL round-bottom flask purged with and maintained under nitrogen was placed 2-bromo-6-cyclopropyl-4-fluorobenzenamine (3.4 g, 14.8 mmol), dioxane (100 mL), water (10 mL), Cs₂CO₃ (14.5 g, 44.5 mmol), 4,4,5,5-tetramethyl-2-(prop-1-en-2-yl)-1,3,2-dioxaborolane (3.75 g, 22.3 mmol), and Pd(dppf)Cl₂ (1.1 g, 1.50 mmol). The resulting solution was stirred overnight at 110° C. and then concentrated under vacuum. The residue was applied onto a silica gel column and eluted with ethyl acetate/petroleum ether (1:40 to 1:20). This resulted in 1.7 g (60%) of the title 10 compound as light yellow oil. MS-ESI: 192.1 (M+1).

### Step 3: 2-Cyclopropyl-4-fluoro-6-isopropylbenzenamine

Into a 250-mL round-bottom flask was placed 2-cyclopropyl-4-fluoro-6-(prop-1-en-2-yl)benzenamine (1.7 g, 8.89 mmol), and MeOH (100 mL). Then Pd/C (10% wt, 100 mg) was added. The flask was evacuated and flushed three times with hydrogen. The resulting solution was stirred for 3 h at RT under an atmosphere of hydrogen. The solids were filtered out. The resulting mixture was concentrated under vacuum. This resulted in 1.53 g (89%) of the title compound as yellow oil. MS-ESI: 194.1 (M+1).

Steps 4-6 used similar procedures for converting compound 46 to Intermediate 14 shown in Scheme 13 to afford Intermediate 17. MS-ESI: 235.1 (M−1).

TABLE 7
The Intermediates in the following Table were prepared using the similar procedures for converting
compound 44 to Intermediate 17 shown in Scheme 15 from appropriate starting materials.

| | | | Exact Mass |
|---|---|---|---|
| Intermediate # | Structure | IUPAC Name | [M − H]⁺ |
| Intermediate 18 | 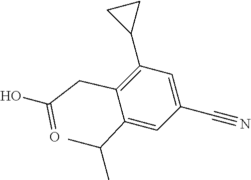 [SMILES: CC(C)c1cc(C#N)cc(C2CC2)c1CC(=O)O] | 2-(4-cyano-2-cyclopropyl-6- isopropylphenyl)acetic acid | 242.1 |
| Intermediate 19 | 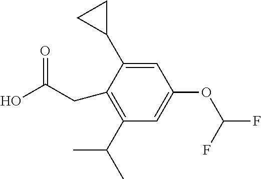 [SMILES: CC(C)c1cc(OC(F)F)cc(C2CC2)c1CC(=O)O] | 2-(2-cyclopropyl-4- (difluoromethoxy)-6- isopropylphenyl)acetic acid | 283.1 |

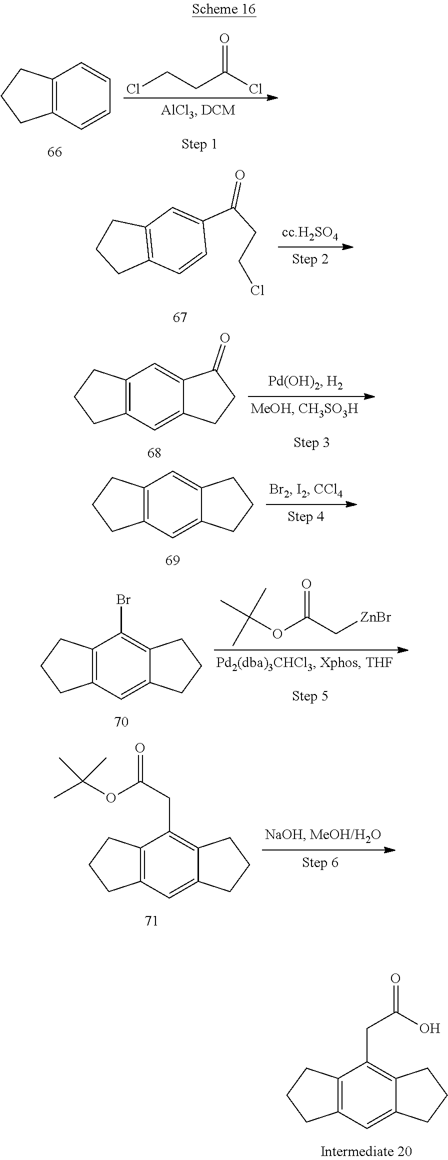

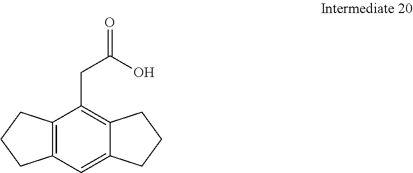 [SMILES: O=C(O)Cc1c2c(cc3c1CCC3)CCC2]

### 2-(1,2,3,5,6,7-Hexahydro-s-indacen-4-yl)acetic acid

### Step 1: 3-Chloro-1-(2,3-dihydro-1H-inden-5-yl)propan-1-one

Into a 1000-mL round-bottom flask was placed a solution of AlCl₃ (37 g, 278 mmol) in DCM (400 mL). This was followed by the addition of a solution of 2,3-dihydro-1H-indene (30 g, 254 mmol) and 3-chloropropanoyl chloride (32.1 g, 253 mmol) in DCM (100 mL) dropwise with stirring at −10° C. in 30 min. The resulting solution was stirred for 16 h at RT. Then the reaction mixture was added dropwise to cold HCl (3 N, 400 mL) over 45 min at −10° C. The resulting solution was extracted with 3×200 mL of DCM; the organic layers combined, dried over anhydrous Na₂SO₄, and concentrated under vacuum. This resulted in 53.5 g (crude) of the title compound as a yellow solid. The crude product was used in the next step.

### Step 2: 1,2,3,5,6,7-Hexahydro-s-indacen-1-one

Into a 1000-mL round-bottom flask was placed a solution of 3-chloro-1-(2,3-dihydro-1H-inden-5-yl)propan-1-one (53.5 g, 253 mmol) in conc. H₂SO₄ (300 mL). The resulting solution was stirred for 16 h at 55° C. and was then quenched by adding the reaction mixture carefully to 1500 mL of water/ice. The solids were collected by filtration and then was dried over infrared lamp for 24 h. This resulted in 37.4 g (85%) of the title compound as a yellow solid.

### Step 3: 1,2,3,5,6,7-Hexahydro-s-indacene

Into a 1000-mL round-bottom flask was placed a solution of 1,2,3,5,6,7-hexahydros-indacen-1-one (37.2 g, 216 mmol), MeOH (300 mL), and CH₃SO₃H (42 g, 437.5 mmol). Then Pd(OH)₂/C (20% wt., 8 g) was added. The flask was evacuated and flushed three times with hydrogen. The resulting solution was stirred for 16 h at RT under an atmosphere of hydrogen. The solids were filtered out. The resulting mixture was concentrated under vacuum. The residue was applied onto a silica gel column and eluted with ethyl acetate/petroleum ether (1:150 to 1:100). This resulted in 27.1 g (79%) of the title compound as a white solid.

### Step 4: 4-Bromo-1,2,3,5,6,7-hexahydro-s-indacene

Into a 500-mL 3-necked round-bottom flask purged with and maintained under nitrogen was placed a solution of 1,2,3,5,6,7-hexahydro-s-indacene (15 g, 94.8 mmol) in CCl₄ (200 mL). Then I₂ (1.2 g, 4.72 mmol) was added. This was followed by the addition of a solution of Br₂ (16 g, 100 mmol) in CCl₄ (50 mL) dropwise with stirring at 0° C. in 10 min. The resulting solution was stirred for 2 h at 0° C. The reaction was then quenched by the addition of 150 mL of NH₄Cl (sat.). The resulting solution was extracted with 3×150 mL of DCM and the organic layers combined and dried over anhydrous Na₂SO₄, then concentrated under vacuum. The crude product was applied onto a silica gel column and eluted with ethyl acetate/petroleum ether. This resulted in 18.0 g (80%) of the title compound as yellow oil. ¹H NMR (300 MHz, DMSO-d₆) δ 7.02 (s, 1H), 2.95-2.75 (m, 8H), 2.03-2.01 (m, 4H).

### Step 5: Tert-butyl 2-(1,2,3,5,6,7-hexahydro-s-indacen-4-yl)acetate

Into a 100-mL round-bottom flask purged with and maintained under nitrogen was placed a solution of 4-bromo-1,2,3,5,6,7-hexahydro-s-indacene (1 g, 4.2 mmol) in THF (20 mL). Then X-phos (200 mg, 0.42 mmol) and Pd₂(dba)₃CHCl₃ (220 mg, 0.21 mmol) were added. The resulting solution was stirred for 10 min at RT. This was followed by the addition of tert-butyl 2-(bromozincio)acetate (2.2 g, 8.45 mmol). The resulting solution was stirred for 4 h at 80° C. and was then quenched by the addition of 50 mL of NH₄Cl (sat.). The resulting solution was extracted with 3×100 mL of DCM. The organic layers were combined, dried over anhydrous Na₂SO₄, and concentrated under vacuum. This resulted in 1.4 g (crude) of the title compound as brown oil. ¹H NMR (400 MHz, DMSO-d₆) δ 6.96 (s, 1H), 3.47 (s, 2H), 2.80-2.78 (m, 8H), 2.01-1.99 (m, 4H), 1.39 (s, 9H).

### Step 6: 2-(1,2,3,5,6,7-hexahydro-s-indacen-4-yl)acetic acid

Into a 40-mL sealed tube was placed a solution of tert-butyl 2-(1,2,3,5,6,7-hexahydro-s-indacen-4-yl)acetate (1.4 g, 5.14 mmol) in 6 M sodium hydroxide/MeOH (4/6 mL). The resulting solution was stirred for 16 h at 100° C. The reaction was then quenched by the addition of 20 mL of water. The resulting solution was extracted with 2×30 mL of DCM and the aqueous layers combined. The pH value of the solution was adjusted to 2 with hydrogen chloride (1 N). The resulting solution was extracted with 3×50 mL of ethyl acetate and the organic layers combined and dried over anhydrous Na₂SO₄, then concentrated under vacuum. This resulted in 180 mg (19.8%, 2 steps) of the title compound as a yellow solid. MS-ESI: 215.1 (M−1).

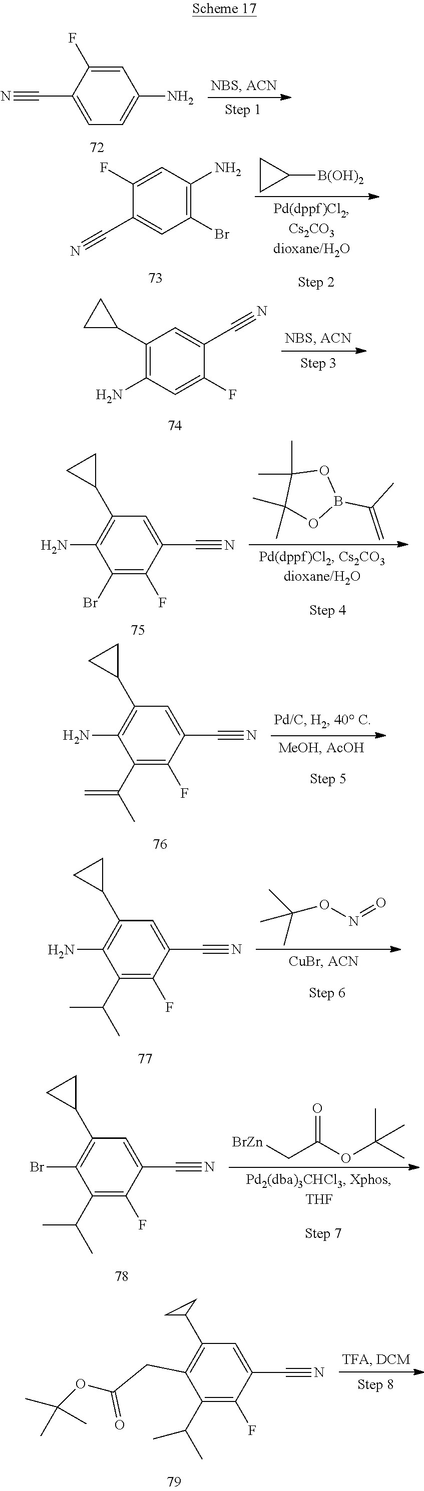

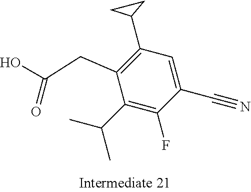 [SMILES: CC(C)c1c(F)c(C#N)cc(C2CC2)c1CC(=O)O]

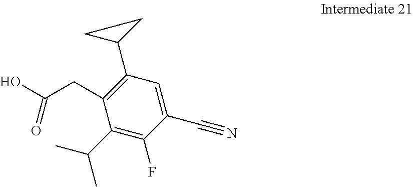 [SMILES: CC(C)c1c(F)c(C#N)cc(C2CC2)c1CC(=O)O]

### 2-(4-Cyano-6-cyclopropyl-3-fluoro-2-isopropylphenyl)acetic acid

### Step 1: 4-Amino-5-bromo-2-fluorobenzonitrile

Into a 250-mL round-bottom flask was placed a solution of 4-amino-2-fluorobenzonitrile (9 g, 66.1 mmol) in ACN (120 mL). Then NBS (12.4 g, 69.7 mmol) was added. The resulting solution was stirred overnight at 80° C. and then was concentrated under vacuum. The residue was applied onto a silica gel column and eluted with ethyl acetate/petroleum ether (1:20 to 1:10). This resulted in 10.9 g (77%) of the title compound as a yellow solid. MS-ESI: 215.0/217.0 (M+1). ¹H NMR (300 MHz, DMSO-d₆) δ 7.89 (d, 0.1=6.0 Hz, 1H), 6.69 (br s, 2H), 6.63 (d, J=12.0 Hz, 1H).

### Step 2: 4-Amino-5-cyclopropyl-2-fluorobenzonitrile

Into a 250-mL round-bottom flask purged with and maintained under nitrogen was placed 4-amino-5-bromo-2-fluorobenzonitrile (6.37 g, 29.6 mmol), 1,4-dioxane (70 mL), water (10 mL), Cs₂CO₃ (9.7 g, 29.8 mmol), cyclopropylboronic acid (3.8 g, 44.2 mmol), and Pd(dppf)Cl₂ (1.08 g, 1.48 mmol). The resulting solution was stirred overnight at 90° C. and then was concentrated under vacuum. The residue was applied onto a silica gel column and eluted with ethyl acetate/petroleum ether (1:10 to 1:5). This resulted in 5.03 g (96%) of the title compound as a yellow solid. MS-ESI: 177.1 (M+1).

### Step 3: 4-Amino-3-bromo-5-cyclopropyl-2-fluorobenzonitrile

Into a 250-mL round-bottom flask was placed 4-amino-5-cyclopropyl-2-fluorobenzonitrile (5.03 g, 28.7 mmol), ACN (50 mL), and NBS (5.6 g, 31.5 mmol). The resulting solution was stirred overnight at 80° C. and then was concentrated under vacuum. The residue was applied onto a silica gel column and eluted with ethyl acetate/petroleum ether (1:10 to 1:5). This resulted in 6.972 g (96%) of the title compound as a yellow solid. MS-ESI: 255.0/257.0 (M+1).

### Step 4: 4-Amino-5-cyclopropyl-2-fluoro-3-(prop-1-en-2-yl)benzonitrile

Into a 250-mL round-bottom flask purged with and maintained under nitrogen was placed 4-amino-3-bromo-5-cyclopropyl-2-fluorobenzonitrile (6.972 g, 27.33 mmol), 1,4-dioxane (120 mL), water (20 mL), 4,4,5,5-tetramethyl-2-(prop-1-en-2-yl)-1,3,2-dioxaborolane (6.9 g, 41.0 mmol), Cs₂CO₃ (13.4 g, 41.0 mmol), and Pd(dppf)Cl₂ (0.4 g, 0.55 mmol). The resulting solution was stirred overnight at 80° C. and was then concentrated under vacuum. The residue was applied onto a silica gel column and eluted with ethyl acetate/petroleum ether (1:10 to 1:5). This resulted in 4.73 g (80%) of the title compound as a yellow solid. MS-ESI: 217.1 (M+1).

### Step 5: 4-Amino-5-cyclopropyl-2-fluoro-3-isopropylbenzonitrile

Into a 250-mL round-bottom flask was placed 4-amino-5-cyclopropyl-2-fluoro-3-(prop-1-en-2-yl)benzonitrile (4.73 g, 21.97 mmol), MeOH (100 mL), and AcOH (0.5 mL). Then Pd/C (10% wt, 500 mg) was added. The flask was evacuated and flushed three times with hydrogen. The resulting solution was stirred for 4 h at 40° C. under an atmosphere of hydrogen. The solids were filtered out. The filtrate was concentrated under vacuum. This resulted in 4.71 g (99%) of the title compound as a light yellow solid. MS-ESI: 219.1 (M+1).

Steps 6-8 used similar procedures for converting compound 46 to Intermediate 14 shown in Scheme 13 to afford Intermediate 21. MS-ESI: 260.1 (M−1).

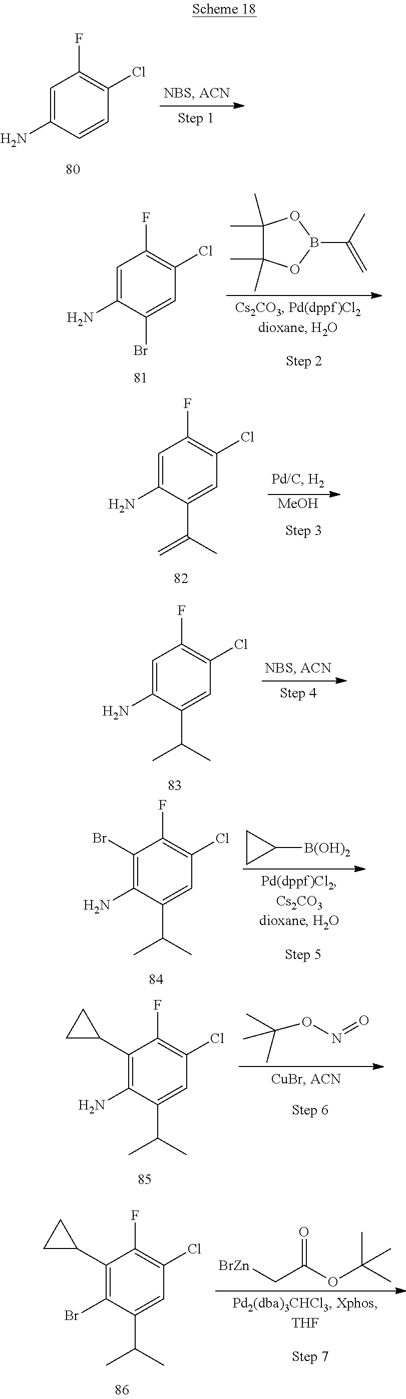

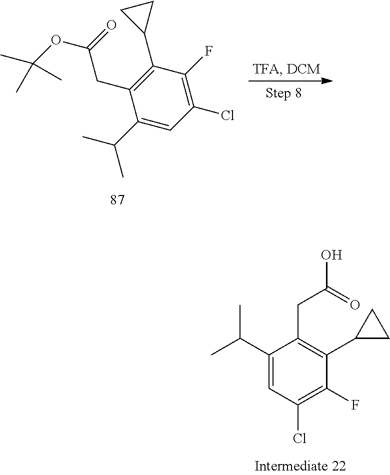 [SMILES: CC(C)c1cc(Cl)c(F)c(C2CC2)c1CC(=O)OC(C)(C)C ; CC(C)c1cc(Cl)c(F)c(C2CC2)c1CC(=O)O]

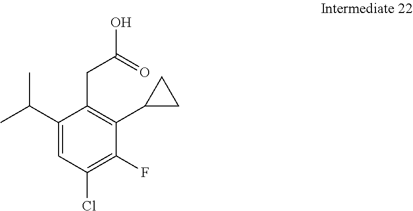 [SMILES: CC(C)c1cc(Cl)c(F)c(C2CC2)c1CC(=O)O]

### 2-(4-Chloro-2-cyclopropyl-3-fluoro-6-isopropylphenyl)acetic acid

### Step 1: 2-Bromo-4-chloro-5-fluorobenzenamine

Into a 1000-mL round-bottom flask was placed 4-chloro-3-fluorobenzenamine (20 g, 137 mmol, ACN (500 mL), and NBS (21.9 g, 123 mmol). The resulting solution was stirred overnight at RT and then was concentrated under vacuum. The residue was applied onto a silica gel column and eluted with ethyl acetate/petroleum ether (1:150 to 1:100). This resulted in 26.3 g (85%) of the title compound as a white solid. MS-ESI: 225.9/223.9/227.9 (M+1). ¹H NMR (300 MHz, CDCl₃-d) δ 7.44 (d, J=8.0 Hz, 1H), 6.59 (d, J=8.8 Hz, 1H), 4.21 (s, 2H).

### Step 2: 4-Chloro-5-fluoro-2-(prop-1-en-2-yl)benzenamine

Into a 1000-mL round-bottom flask purged with and maintained under nitrogen was placed 2-bromo-4-chloro-5-fluorobenzenamine (26.3 g, 117 mmol), 1,4-dioxane (500 mL), water (50 mL), 4,4,5,5-tetramethyl-2-(prop-1-en-2-yl)-1,3,2-dioxaborolane (23.7 g, 141 mmol), Cs₂CO₃ (76.6 g, 235 mmol), and Pd(dppf)Cl₂ (1.71 g, 2.34 mmol). The resulting solution was stirred overnight at 90° C. and was then concentrated under vacuum. The residue was applied onto a silica gel column and eluted with ethyl acetate/petroleum ether (1:150 to 1:100). This resulted in 12.6 g (58%) of the title compound as brown oil. MS-ESI: 186.0/188.0 (M+1).

### Step 3: 4-Chloro-5-fluoro-2-isopropylbenzenamine

Into a 500-mL round-bottom flask was placed 4-chloro-5-fluoro-2-(prop-1-en-2-yl)benzenamine (12.6 g, 67.88 mmol) in MeOH (250 mL). Then Pd/C (10% wt, 1.2 g) was added. The flask was evacuated and flushed three times with hydrogen. The resulting solution was stirred for 3 h at RT under an atmosphere of hydrogen. The solids were filtered out. The filtrate was concentrated under vacuum. This resulted in 12.5 g (98%) of the title compound as light yellow oil. MS-ESI: 188.1/190.1 (M+1).

### Step 4: 2-Bromo-4-chloro-3-fluoro-6-isopropylbenzenamine

Into a 500-mL round-bottom flask was placed 4-chloro-5-fluoro-2-isopropylbenzenamine (6 g, 32.0 mmol), ACN (200 mL), and NBS (6.25 g, 35.1 mmol). The resulting solution was stirred for 3 h at RT and then was concentrated under vacuum. The residue was applied onto a silica gel column and eluted with ethyl acetate/petroleum ether (1:150 to 1:100). This resulted in 8 g (94%) of the title compound as brown oil. MS-ESI: 268.0/266.0/270.0 (M+1).

### Step 5: 4-Chloro-2-cyclopropyl-3-fluoro-6-isopropylbenzenamine

Into a 100-mL round-bottom flask purged with and maintained under nitrogen was placed 2-bromo-4-chloro-3-fluoro-6-isopropylbenzenamine (2.9 g, 10.9 mmol), 1,4-dioxane (40 mL), water (8 mL), cyclopropylboronic acid (1.12 g, 13.0 mmol), Cs₂CO₃ (7.08 g, 21.7 mmol), and Pd(dppf)Cl₂ (795 mg, 1.09 mmol). The resulting solution was stirred for 3 h at 90° C. and then was concentrated under vacuum. The residue was applied onto a silica gel column and eluted with ethyl acetate/petroleum ether (1:80 to 1:50). This resulted in 1.1 g (44%) of the title compound as light brown oil. MS-ESI: 228.1/230.1 (M+1).

Steps 6-8 used similar procedures for converting compound 46 to Intermediate 14 shown in Scheme 13 to afford Intermediate 22. MS-ESI: 271.1/273.1 (M−1).

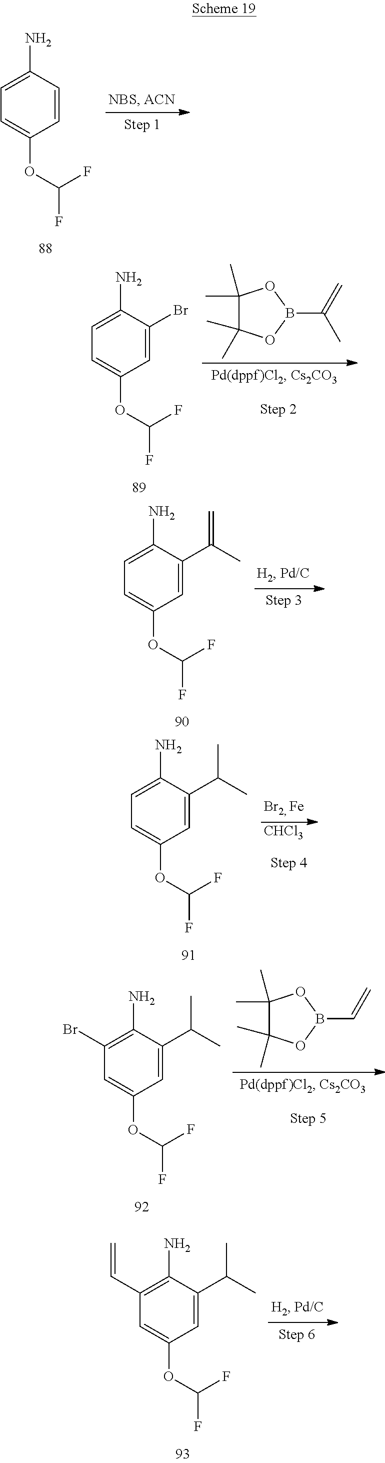

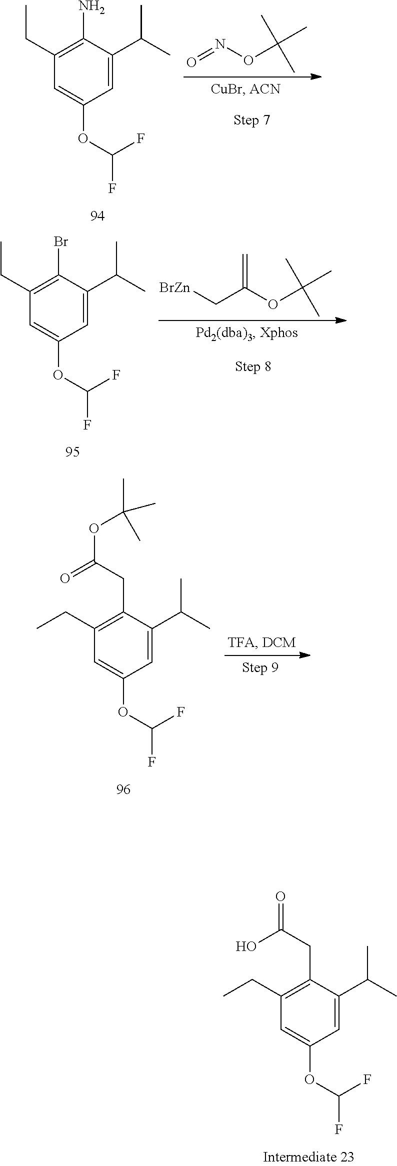

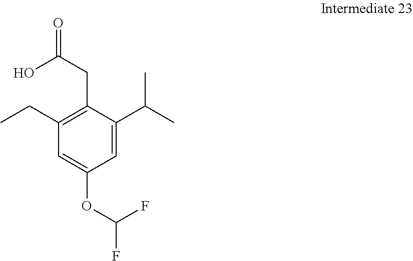 [SMILES: CCc1cc(OC(F)F)cc(C(C)C)c1CC(=O)O]

### 2-(4-(Difluoromethoxy)-2-ethyl-6-isopropylphenyl)acetic acid

Steps 1-3 used similar procedures for converting compound 80 to compound 83 shown in Scheme 18 to afford compound 91. MS-ESI: 202.1 (M+1).

### Step 4: 2-Bromo-4-(difluoromethoxy)-6-isopropylbenzenamine

Into a 250-mL round-bottom flask was placed a mixture of 4-(difluoromethoxy)-2-isopropylbenzenamine (2.01 g, 10 mmol) and iron powder (L 12 g, 20 mmol) in CHCl₃ (50 mL). To this was added bromine (1.23 mL, 24 mmol). The resulting solution was stirred for 6 h at RT and diluted with water (200 mL). The mixture was extracted with 3×50 mL of ethyl acetate. The organic layers were combined, dried over Na₂SO₄, and then concentrated under vacuum. The residue was applied onto a silica gel column and eluted with ethyl acetate/petroleum ether (1:100 to 1:10). This resulted in 2.24 g (80%) of the title compound as a yellow solid. MS-ESI: 280.0/282.0 (M+1).

Steps 5-9 used similar procedures for converting compound 44 to intermediate 14 shown in Scheme 13 to afford Intermediate 23. MS-ESI: 271.1 (M−1).

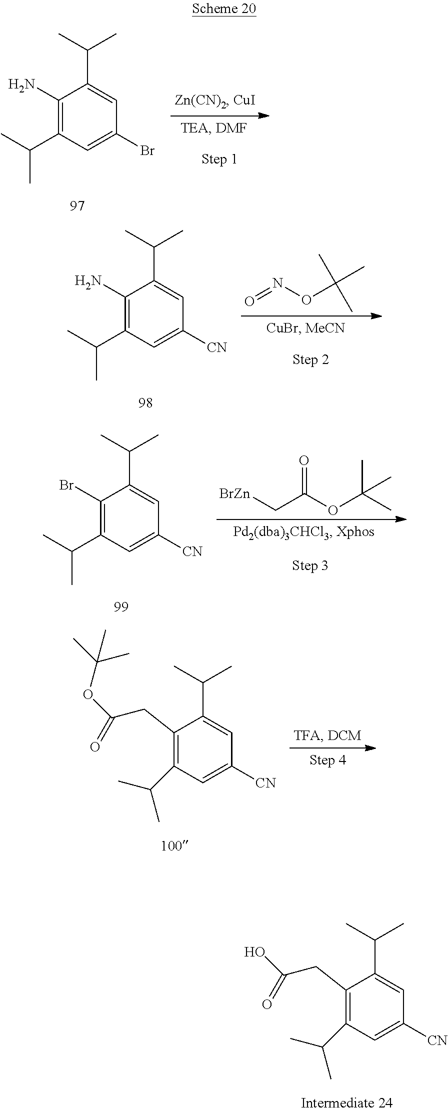

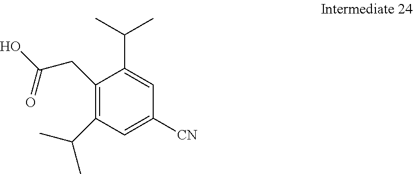 [SMILES: CC(C)c1cc(C#N)cc(C(C)C)c1CC(=O)O]

### 2-(4-Cyano-2,6-diisopropylphenylacetic acid

### Step 1: 4-Amino-3,5-diisopropylbenzonitrile

Into a 100-mL round-bottom flask purged with and maintained under nitrogen was placed a solution of 4-bromo-2,6-diisopropylbenzenamine (commercially available, 5.1 g, 19.9 mmol) in DMF (30 mL). To the solution were added Zn(CN)₂ (2.80 g, 23.9 mmol), CuI (380 mg, 2.00 mmol), and TEA (3.0 g, 29.9 mmol). The resulting solution was stirred for 16 h at 120° C. and then was diluted with 30 mL of water. The solution was extracted with 3×30 mL of ethyl acetate and the combined organic layers were concentrated under vacuum. The residue was applied onto a silica gel column and eluted with ethyl acetate/petroleum ether (1:30 to 1:20). This resulted in 2.4 g (60%) of the title compound as a yellow solid. MS-ESI: 203.1 (M+1).

Steps 2-4 used similar procedures for converting compound 46 to Intermediate 14 shown in Scheme 13 to afford Intermediate 24. MS-ESI: 244.1 (M−1).

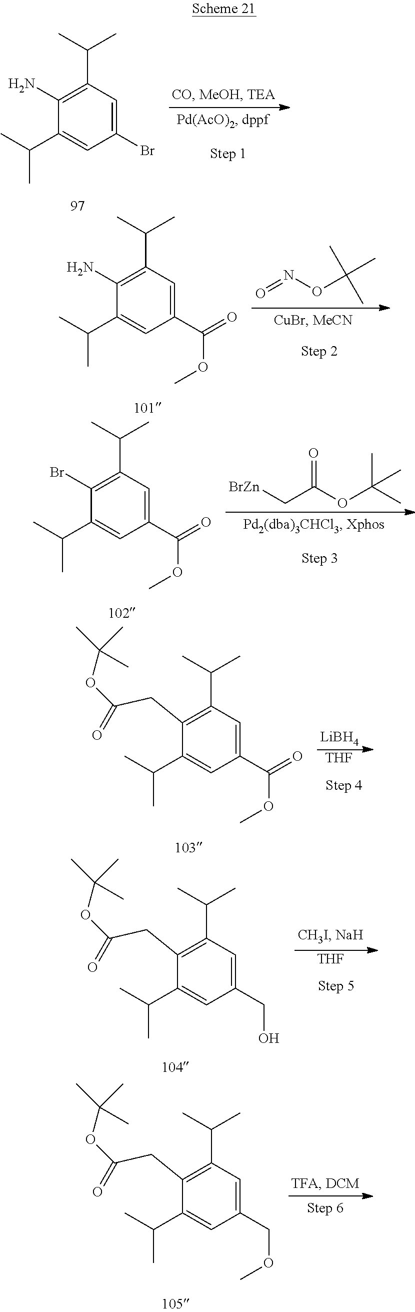

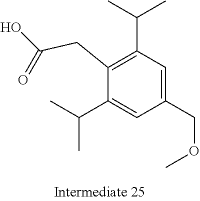 [SMILES: COCc1cc(C(C)C)c(CC(=O)O)c(C(C)C)c1]

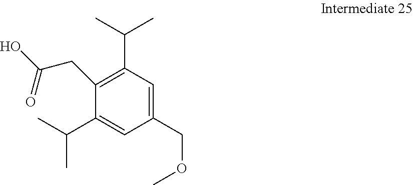 [SMILES: COCc1cc(C(C)C)c(CC(=O)O)c(C(C)C)c1]

### 2-(2,6-diisopropyl-4-(methoxymethyl)phenyl)acetic acid

### Step 1: Methyl 4-amino-3,5-diisopropylbenzoate

Into a 1-L autoclave was placed a solution of 4-bromo-2,6-diisopropylbenzenamine (10 g, 39 mmol) in MeOH (300 mL). To the solution were added Pd(OAc)2 (1.75 g, 7.8 mmol), dppf (4.3 g, 7.8 mmol), and TEA (20 g, 195 mmol). After sealing the autoclave, the gas was exchanged with CO for 3 times. The reaction was stirred at 120° C. for overnight. After cooling the reaction mixture, the gas was exchanged with N2, the reaction was concentrated and diluted with water (300 mL). The resulting solution was extracted with EtOAc (3×200 mL). The combined organic layers were dried over anhydrous Na₂SO₄ and concentrated. The residue was purified on SiO₂-gel column and eluted with ethyl acetate/petroleum ether (1:10 to 1:5). This resulted in 5.6 g (62%) of the title compound as a brown oil. MS-ESI: 236.2 (M+1) Steps 2 and 3 used similar procedures for converting compound 46 to 48, shown in Scheme 13 to afford compound 103″ as colorless oil. 335.2 (M+1)

### Step 4: Tert-butyl 2-(4-(hydroxymethyl)-2,6-diisopropylphenyl)acetate

Into a 100 mL round bottom flask was placed a solution of methyl 4-(2-tert-butoxy-2-oxoethyl)-3,5-diisopropylbenzoate (2 g, 6.0 mmol) in THF (25 mL). LiBH₄ (264 mg, 12.0 mmol) was added to the mixture at 0° C. in portions, and the mixture was stirred at 0° C. for 1h. The reaction was quenched with ice-water (20 mL). The solution was extracted with EtOAc (3×100 mL); the combined organic layers were dried over anhydrous Na₂SO₄ and concentrated under vacuum. The residue was purified with SiO₂-gel column and eluted with ethyl acetate/petroleum ether (1:5 to 1:2). This resulted in 1.1 g (60%) of the title compound as a white solid. MS-ESI: 307.2 (M+1).

### Step 5: Tert-butyl 2-(2,6-diisopropyl-4-(methoxymethyl)phenyl)acetate

Into a 100 mL round bottom flask was placed a solution of tert-butyl 2-(4-(hydroxymethyl)-2,6-diisopropylphenyl)acetate (1.1 g, 3.6 mmol) in THF (20 mL). NaH (60% wt., 173 mg, 4.3 mmol) was added to the mixture at 0° C. in portions, and the mixture was stirred at 0° C. for 30 min. MeI (1.0 g, 7.2 mmol) was added to the mixture dropwise at 0° C.; the resulting mixture was stirred at RT for overnight. The reaction was quenched with ice-water (20 mL) and extracted with EtOAc (3×100 mL). The combined organic layer were dried over Na₂SO₄ and concentrated under vacuum. The residue was purified with SiO₂-gel column and eluted with ethyl acetate/petroleum ether (1:10-1:5). This resulted in 1.1 g (95%) of title compound as a colorless oil. MS-ESI: 321.2 (M+1).

### Step 6: 2-(2,6-Diisopropyl-4-(methoxymethyl)phenyl)acetic acid

Into a 50-mL round-bottom flask was placed a solution of tert-butyl 2-[4-fluoro-2,6-bis(propan-2-yl)phenyl]acetate (1.1 g, 3.4 mmol) in DCM (10 mL) and TFA (10 mL). The resulting solution was stirred for 3 h at RT and then was concentrated under vacuum. This resulted in 1.0 g (crude) of the title compound as a light yellow solid. MS-ESI: 263.2 (M−1).

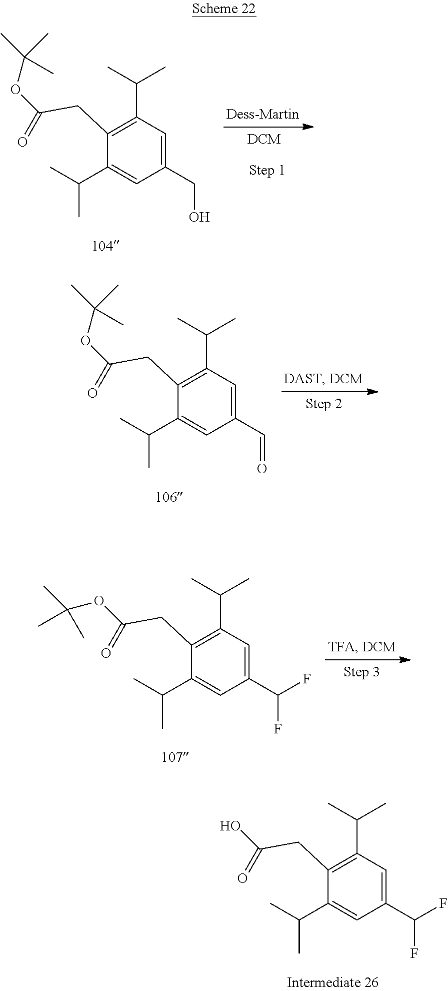 [SMILES: CC(C)c1cc(CO)cc(C(C)C)c1CC(=O)OC(C)(C)C ; CC(C)c1cc(C=O)cc(C(C)C)c1CC(=O)OC(C)(C)C ; CC(C)c1cc(C(F)F)cc(C(C)C)c1CC(=O)OC(C)(C)C ; CC(C)c1cc(C(F)F)cc(C(C)C)c1CC(=O)O]

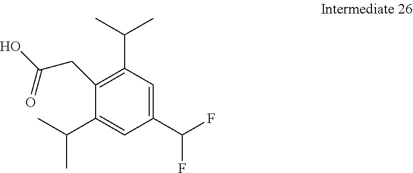 [SMILES: CC(C)c1cc(C(F)F)cc(C(C)C)c1CC(=O)O]

### 2-(4-(Difluoromethyl)-2,6-diisopropylphenyl)acetic acid

### Step 1: Tert-butyl 2-(4-formyl-2,6-diisopropylphenyl)acetate

Into a 100 mL round bottom flask was placed a solution of tert-butyl 2-(4-(hydroxymethyl)-2,6-diisopropylphenyl)acetate (1.1 g, 3.6 mmol) in DCM (20 mL). Dess-Martin Periodinane (2.29 g, 5.4 mmol) was added to the mixture at 0° C. in portions. The mixture was stirred at RT overnight, after which the reaction was quenched with ice-water (20 mL) and extracted with DCM (3×50 mL). The combined organic layers were dried over anhydrous Na₂SO₄ and concentrated under vacuum. The residue was purified on a SiO₂-gel column and eluted with ethyl acetate/petroleum ether (1:20-1:10). This resulted in 0.98 g (90%) of title compound as a yellow solid. MS-ESI: 305.2 (M+1).

### Step 2: Tert-butyl 2-(4-(difluoromethyl)-2,6-diisopropylphenyl)acetate

Into a 100 mL round bottom flask was placed a solution of tert-butyl 2-(4-formyl-2,6-diisopropylphenyl)acetate (912 mg, 3.0 mmol) in DCM (15 mL). DAST (2.41 g, 15 mmol) was added to the mixture at 0° C. in portions. The mixture was stirred at RT overnight, after which the reaction was quenched with water (10 mL) and extracted with DCM (3×30 mL). The combined organic layers were dried over Na₂SO₄ and concentrated under vacuum. The residue was purified with SiO₂-gel column and eluted with ethyl acetate/petroleum ether (1:20-1:15). This resulted in 586 mg (60%) of title compound as a yellow solid. MS-ESI: 327.2 (M+1).

Steps 3 used similar procedures for converting compound 105″ to Intermediate 25 shown in Scheme 21 to afford Intermediate 26. MS-ESI: 269.1 (M−1).

Schemes of Sulfonimidoylamide Intermediates: Schemes 23-30 illustrate the preparation of sulfonimidoylamide intermediates.

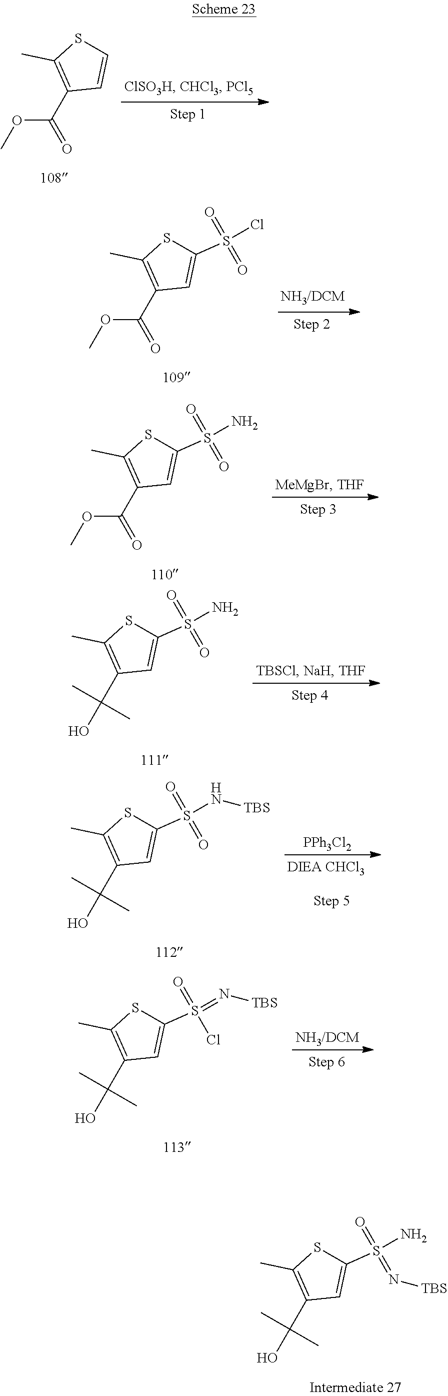

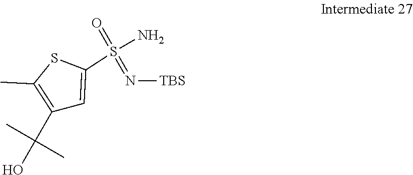

### N′-(tert-butyldimethylsilyl)-4(2-hydroxypropan-2-yl)-5-methylthiophene-2-sulfonimidamide

### Step 1: Methyl 5-(chlorosulfonyl)-2-methylthiophene-3-carboxylate

Into a 250-mL round-bottom flask, was placed methyl 2-methylthiophene-3-carboxylate (5.0 g, 32.0 mmol), CHCl₃ (70 mL). This was followed by the addition of ClSO₂OH (5.6 g, 48.0 mmol) dropwise with stirring. To this was added PCl₅ (13.3 g, 64.0 mmol) with stirring. The resulting 15 solution was stirred for 2 h at 60° C. in an oil bath. The reaction was then quenched by the addition of 150 mL of water/ice. The resulting solution was extracted with 3×80 ml of dichloromethane, dried over anhydrous sodium sulfate, and concentrated. This resulted in 5.2 g (63.8%) of the title compound as a yellow solid.

### Step 2: Methyl 2-methyl-5-sulfamoylthiophene-3-carboxylate

Into a 250-mL round-bottom flask, was placed methyl 5-(chlorosulfonyl)-2-methylthiophene-3-carboxylate (5.2 g, 20.4 mmol) in DCM (50 mL), to this solution was added NH₃/DCM (50 mL, sat.) dropwise with stirring. The resulting solution was stirred for 2 h at 40° C. in an oil bath. The resulting mixture was concentrated. The residue was eluted from a silica gel with ethyl acetate/petroleum ether (2:3). This resulted in 4.6 g (95.8%) of the title compound as a yellow solid. MS-ESI: 236 [M+1].

### Step 3: 4-(2-Hydroxypropan-2-yl)-5-methylthiophene-2-sulfonamide

Into a 250-mL 3-necked round-bottom flask purged and maintained with an inert atmosphere of nitrogen, was placed methyl 2-methyl-5-sulfamoylthiophene-3-carboxylate (4.6 g, 19.5 mmol) in THF (100 mL). This was followed by the addition of MeMgBr (29 mL, 87 mmol, 3M) dropwise with stirring at 0° C. in an ice bath. The resulting solution was stirred for 2 h at RT. The pH value of the solution was adjusted to 5 with HCl (2 M). The resulting solution was extracted with 3×100 ml of ethyl acetate, dried over anhydrous sodium sulfate, and concentrated. The residue was eluted from silica gel with ethyl acetate/petroleum ether (1:2). This resulted in 1.3 g (28.2%) of the title 15 compound as a light yellow solid. MS-ESI: 236 [M+1].

### Step 4: N-(tert-butyldimethylsilyl)-4-(2-hydroxypropan-2-yl)-5-methylthiophene-2-sulfonamide

Into a 100-mL round-bottom flask, was placed 4-(2-hydroxypropan-2-yl)-5-methylthiophene-2-sulfonamide (1.3 g, 5.52 mmol) in THF (40 mL). To this solution was added NaH (60% wt. oil dispersion, 442 mg, 11.1 mmol) in portions with stirring at 0° C. This was followed by the addition of TBSCl (1.25 g, 8.29 mmol). The resulting solution was stirred for 1 h at RT. The reaction was then quenched by the addition of 50 mL of NH₄Cl solution. The resulting solution was extracted with 3×50 ml of ethyl acetate, dried over anhydrous sodium sulfate, and concentrated. The residue was eluted from silica gel with ethyl acetate/petroleum ether (1:2). This resulted in 1.2 g (62.1%) of the title compound as a white solid. MS-ESI: 350[M+1].

### Steps 5 and 6: N′-(tert-butyldimethylsilyl)-4-(2-hydroxypropan-2-yl)-5-methylthiophene-2-sulfonimidamide

Into a 100-mL 3-necked round-bottom flask purged and maintained with an inert atmosphere of nitrogen, was placed a solution of PPh₃Cl₂ (3.51 g, 10.5 mmol) in CHCl₃ (40 mL). This was followed by the addition of DIEA (1.77 g, 13.7 mmol) dropwise with stirring at RT. The resulting solution was stirred for 10 min at RT and the reaction mixture was cooled to 0° C. To this was added a solution of N-(tert-butyldimethylsilyl)-4-(2-hydroxypropan-2-yl)-5-methylthiophene-2-sulfonamide (1.2 g, 3.43 mmol) in CHCl₃ (5 mL) dropwise with stirring at 0° C. The resulting solution was stirred for 30 min at 0° C. To the mixture was introduced NH₃ gas bubble for 15 min at 0° C. The resulting solution was stirred for 2 h at RT. The resulting solution was diluted with 50 mL of H₂O. The resulting solution was extracted with 3×100 ml of DCM and dried over anhydrous sodium sulfate and concentrated. The residue was eluted from silica gel with ethyl acetate/petroleum ether (1:1). This resulted in 930 mg (77.7%) of the title compound as a yellow solid. MS-ESI: 349 [M+1].

TABLE 12
The Intermediates in the following Table were prepared using similar procedure as
shown in Scheme 23 above for converting compound 108″ to Intermediate 27 starting from methyl
thiophene-3-carboxylate.

| | | | Exact Mass |
|---|---|---|---|
| Intermediate # | Structure | IUPAC Name | [M + H]⁺ |
| Intermediate 28 | 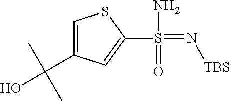 | N′-(tert-butyldimethylsilyl)-4-(2-hydroxypropan- 2-yl)thiophene-2-sulfonimidamide | 335 |

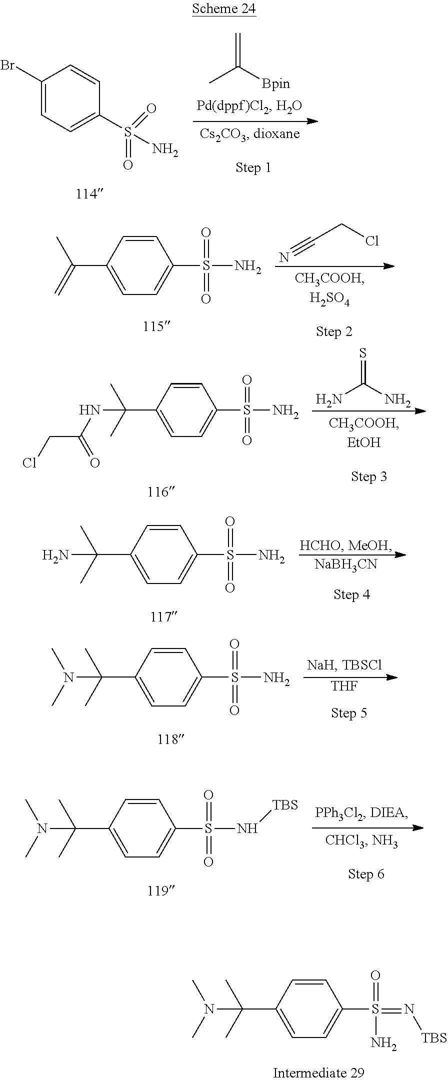

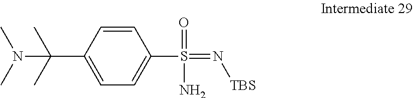

### N′-(tert-butyldimethylsilyl)-4-(2-(dimethylamino)propan-2-yl)benzenesulfonimidamide

### Step 1: 4-(Prop-1-en-2-yl)benzenesulfonamide

Into a 500-mL 3-necked round-bottom flask purged and maintained with an inert atmosphere of nitrogen, was placed a solution of 4-bromobenzene-1-sulfonamide (5.0 g, 21.2 mmol) in dioxane (75 mL) and H₂O (7.5 mL). To this solution was added 4,4, 5, 5-tetramethyl-2-(prop-1-en-2-yl)-1,3,2-dioxaborolane (7.83 g, 46.59 mmol), Pd(dppf)Cl₂ (1.5 g, 2.12 mmol) and Cs₂CO₃ (27.6 g, 84.7 mmol). The resulting solution was stirred for 2 h at 85′C. The resulting solution was diluted with 400 mL of water. The resulting solution was extracted with 2×500 mL of ethyl acetate and dried over anhydrous sodium sulfate and concentrated. The residue was eluted from silica gel with ethyl acetate/petroleum ether (1:3). This resulted in 4.7 g (98.1%) of the title compound as a yellow solid. MS-ESI: 198.1 [M+1].

### Step 2: 2-Chloro-N-(2-(4-sulfamoylphenyl)propan-2-yl)acetamide

Into a 1 L round-bottom flask, was placed a solution of 4-(prop-1-en-2-yl)benzene-1-sulfonamide (2.2 g, 11.2 mmol) in AcOH (280 mL). To the solution was added 2-chloroacetonitrile (16.8 g, 224 mmol). This was followed by the addition of H₂SO₄ (70 mL, 0.7 mmol) dropwise with stirring at 0° C. The resulting solution was stirred overnight at RT. The resulting solution was diluted with 500 mL of water/ice. The pH value of the solution was adjusted to 7 with a saturated solution of Na₂CO₃. The resulting solution was extracted with 3×1000 mL of DCM, dried over anhydrous sodium sulfate and concentrated. The residue was eluted from silica gel with ethyl acetate/petroleum ether (3:2). This resulted in 2.7 g (83.2%) of the crude title compound as a white solid. MS-ESI: 291.0 [M+1].

### Step 3: 4-(2-Aminopropan-2-yl)benzenesulfonamide

Into a 100-mL round-bottom flask, was placed a solution of 2-chloro-N-[2-(4-sulfamoylphenyl)propan-2-yl] acetamide (1.0 g, 3.44 mmol) in ethanol (30 mL) and AcOH (6.0 mL, 99.93 mmol). To the solution was added thiourea (314.2 mg, 4.13 mmol). The resulting solution was stirred for overnight at 85° C. The resulting mixture was concentrated. The resulting mixture was washed with 50 mL of ethanol. The solids were collected by filtration. The solid was 15 dried under infra-red for 16 h. This resulted in 520 mg (70.56%) of the crude title compound as a white solid. MS-ESI: 215.1 [M+1].

### Step 4: 4-(2-(Dimethylamino)propan-2-yl)benzenesulfonamide

Into a 50-mL round-bottom flask, was placed a solution of 4-(2-aminopropan-2-yl)benzene-1-sulfonamide (500 mg, 2.33 mmol) in methanol (20 mL). This was followed by the addition of HCHO (140 mg, 4.67 mmol). The resulting solution was stirred for 30 min at RT. To this was added NaBH₃CN (439 mg, 7.0 mmol) in several batches at 0° C. The resulting solution was stirred for 1 h at RT. The reaction was then quenched by the addition of 50 mL of water. The resulting mixture was concentrated and washed with 20 mL of H₂O. The solids were collected by filtration and dried in an oven under reduced pressure at 50′C. This resulted in 300 mg (53.1%) of the crude title compound as a white solid. MS-ESI: 243.1[M+1].

### Step 5: N-(tert-butyldimethylsilyl)-4-(2-(dimethylamino)propan-2-yl)benzenesulfonamide

Into a 50-mL round-bottom flask, was placed a solution of 4-[2-(dimethylamino)propan-2-yl]benzene-1-sulfonamide (200 mg, 0.83 mmol) in THF (15 mL). This was followed by the addition of NaH (60% wt. oil dispersion, 66 mg, 1.65 mmol) at 0° C. The resulting solution was stirred for 10 min at RT. To this was added TBSCl (497 mg, 3.3 mmol). The resulting solution was stirred for 1 h at RT. The reaction was then quenched by the addition of 60 mL of water/ice. The resulting solution was extracted with 2×60 mL of ethyl acetate and dried over anhydrous sodium sulfate and concentrated. The residue was eluted from silica gel with ethyl acetate/petroleum ether (1:1). This resulted in 243 mg (82.5%) of the title compound as a white solid. MS-ESI: 357.2 [M+1].

### Step 6: N′-(tert-butyldimethylsilyl)-4-(2-(dimethylamino)propan-2-yl)benzenesulfonimidamide

Into a 250-mL 3-necked round-bottom flask purged and maintained with an inert atmosphere of nitrogen, was placed a solution of dichlorotriphenyl-λ5-phosphane (467 mg, 1.4 mmol) in CHCl₃ (30 mL). This was followed by the addition of DIEA (261 mg, 2.02 mmol) dropwise with stirring. The resulting solution was stirred for 15 min at RT, and the reaction system was cooled to 0° C. To this was added a solution of N-(tert-butyldimethylsilyl)-4-[2-(dimethylamino)propan-2-yl]benzene-1-sulfonamide (200 mg, 0.56 mmol) in CHCl₃ (10 mL) dropwise with stirring at 0° C. The resulting solution was stirred for 30 min at 0° C. To the mixture was added a solution of NH₃ in DCM (60 mL, sat.). The resulting solution was stirred for 2 h at RT. The resulting mixture was diluted with 80 mL of H₂O. The resulting solution was extracted with 2×100 mL of DCM and dried over anhydrous sodium sulfate and concentrated. The residue was eluted from silica gel with ethyl acetate/petroleum ether (1.4:1). This resulted in 140 mg (70.1%) of the title compound as a white solid. MS-ESI: 356.2 [M+1].

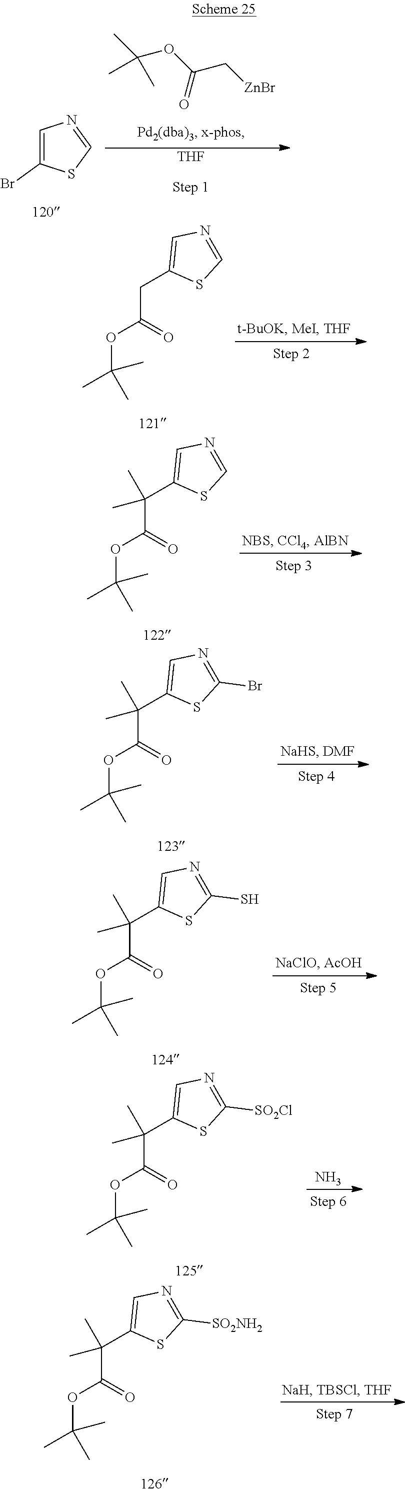

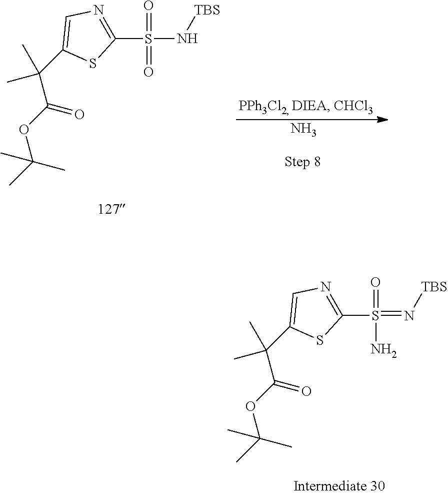

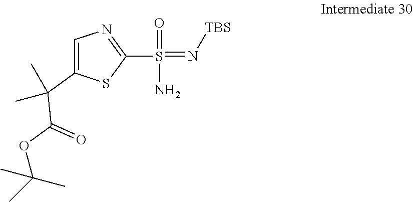

Teri-butyl 2-(2-(N′-(tert-butyldimethylsilyl)sulfamidimidoyl)thiazol-5-yl)-2-methylpropanoate

### Step 1: Tert-butyl 2-(thiazol-5-yl)acetate

Into a 250-mL 3-necked round-bottom flask purged and maintained with an inert atmosphere of nitrogen, was placed a solution of 5-bromo-1,3-thiazole (3.0 g, 18.3 mmol) in THF (100 mL). This was followed by the addition of Pd₂(dba)3 (947 mg, 0.91 mmol) and Xphos (1.05 g, 1.83 mmol). The resulting solution was stirred for 10 min at RT. To this was added tert-butyl 2-(bromozincio)acetate (9.5 g, 36.5 mmol). The resulting solution was stirred for 1.5 h at 60° C. The resulting mixture was diluted with 150 mL of H₂O. The resulting solution was extracted with 2×200 mL of ethyl acetate, dried over anhydrous sodium sulfate and concentrated. The residue was eluted from silica gel with ethyl acetate/petroleum ether (1:5). This resulted in 1.0 g (27.4%) of the title compound as a yellow liquid. MS-ESI: 200.1 [M+1].

### Step 2: Tert-butyl 2-methyl-2-(thiazol-5-yl)propanoate

Into a 250-mL round-bottom flask, was placed a solution of tert-butyl 2-(1,3-thiazol-5-yl)acetate (1.0 g, 5.02 mmol) in THF (50 mL). To the solution were added t-BuOK (2.30 g, 20.4 mmol) and MeI (2.91 g, 20.4 mmol). The resulting solution was stirred for 1 h at RT. The reaction was then quenched by the addition of 200 mL of water. The resulting solution was extracted with 3×200 mL of ethyl acetate dried over anhydrous sodium sulfate and concentrated. The residue was eluted from silica gel with ethyl acetate/petroleum ether (1:5). This resulted in 1.05 g (92.0%) of the title 20 compound as brown yellow oil. MS-ESI: 228.1 [M+1].

### Step 3: Tert-butyl 2-(2-bromothiazol-5-yl)-2-methylpropanoate

Into a 100-mL round-bottom flask purged and maintained with an inert atmosphere of nitrogen, was placed a solution of tert-butyl 2-methyl-2-(1,3-thiazol-5-yl)propanoate (500 mg, 2.2 mmol) in CCl₄ (30 mL). To the solution were added NBS (783 mg, 4.4 mmol) and AIBN (72.2 mg, 0.44 mmol). The resulting solution was stirred for 5 h at 70° C. The reaction was then quenched by the addition of 60 mL of water. The resulting solution was extracted with 2×100 mL of DCM, dried over anhydrous sodium sulfate, and concentrated. The residue was eluted from silica gel with ethyl acetate/petroleum ether (1:6). This resulted in 450 mg (66.9%) of the title compound as yellow oil. MS-ESI: 306.0 [M+1].

### Step 4: Tert-butyl 2-(2-mercaptothiazol-5-yl)-2-methylpropanoate

Into a 50-mL round-bottom flask, was placed a solution of tert-butyl 2-(2-bromo-1,3-thiazol-5-yl)-2-methylpropanoate (450 mg, 1.5 mmol) in DMF (10 mL). To the solution was added NaSH (2.97 g, 30 mmol). The resulting solution was stirred overnight at 100° C. The pH value was adjusted to 6 with 1 M HCl. The resulting solution was washed with 2×25 mL of H₂O and extracted with 2×50 mL of ethyl acetate. The organic layers were combined and dried over anhydrous sodium sulfate. The residue was eluted from silica gel with ethyl acetate/petroleum ether (1:1). This resulted in 350 mg (91.6%) of the title compound as yellow oil. MS-ESI: 260.1 [M+1].

### Step 5: Tert-butyl 2-(2-(chlorosulfonyl)thiazol-5-yl)-2-methylpropanoate

Into a 25-mL round-bottom flask, was placed a solution of tert-butyl 2-methyl-2-(2-sulfanyl-1,3-thiazol-5-yl)propanoate (350 mg, 1.35 mmol) in AcOH (10 mL) at 0° C. To this was added NaClO (10% wt., 5.03 g, 67.4 mmol) dropwise with stirring at 0° C. The resulting solution was stirred for 90 min at RT. The resulting mixture was diluted with 2×100 mL of H₂O. The resulting solution was extracted with 150 mL of DCM, the combined organic layer was dried over anhydrous sodium sulfate and concentrated. This resulted in 100 mg (56.8%) of the title compound as yellow oil.

### Step 6: Tert-butyl 2-methyl-2-(2-sulfamoylthiazol-5-yl)propanoate

Into a 50-mL round-bottom flask, was placed a solution of tert-butyl 2-[2-(chlorosulfonyl)-1,3-thiazol-5-yl]-2-methylpropanoate (100 mg, 0.31 mmol) in DCM (5 mL). To the above solution NH₃ (g) was introduced. The resulting solution was stirred for 20 min at RT. The resulting mixture was concentrated. The residue was eluted from silica gel with ethyl acetate/petroleum ether (3:4). This resulted in 90 mg (95.7%) of the title compound as a white solid. MS-ESI: 307.1 [M+1].

### Step 7: Tert-butyl 2-(2-(N-(tert-butyldimethylsilyl)sulfamoyl)thiazol-5-yl)-2-methylpropanoate

Into a 25-mL round-bottom flask, was placed a solution of tert-butyl 2-methyl-2-(2-sulfamoyl-1,3-thiazol-5-yl)propanoate (50 mg, 0.16 mmol) in THF (5 mL). This was followed by the addition of NaH (60% wt. oil dispersion, 9.6 mg, 0.24 mmol) at 0° C. To this was added TBSCl (49.2 mg, 0.33 mmol). The resulting solution was stirred for 40 min at RT. The reaction was then quenched by the addition of 30 mL of water/ice. The resulting solution was extracted with 2×50 mL of ethyl acetate, dried over anhydrous sodium sulfate and concentrated. The residue was eluted from silica gel with ethyl acetate/petroleum ether (1:3). This resulted in 120 mg (97.10%) of the title compound 5 as a white solid. MS-ESI: 421.2 [M+1].

### Step 8: Tert-butyl 2-(2-(N′-(tert-butyldimethylsilyl)sulfamidimidoyl)thiazol-5-yl)-2-methylpropanoate

Into a 50-mL 3-necked round-bottom flask purged and maintained with an inert atmosphere of nitrogen, was placed a solution of PPh₃Cl₂ (236 mg, 0.71 mmol) in CHCl₃ (15 mL). This was followed by the addition of DIEA (147 mg, 1.14 mmol) dropwise with stirring at 0° C. The resulting solution was stirred for 15 min at RT. To this was added a solution of tert-butyl 2-[2-[(tert-butyldimethylsilyl)sulfamoyl]-1,3-thiazol-5-yl]-2-methylpropanoate (120 mg, 0.29 mmol) in CHCl₃ (4 mL) dropwise with stirring at 0′C. The resulting solution was stirred for 30 min at 0° C. To the above solution was introduced NH₃(g). The resulting solution was stirred for 1 h at RT. The resulting mixture was diluted with 50 mL of H₂O. The resulting solution was extracted with 2×75 mL of DCM, the combined organic layer was dried over anhydrous sodium sulfate and concentrated. The residue was eluted from silica gel with ethyl acetate/petroleum ether (1:6). This resulted in 80 mg (66.6%) of the title compound as a white solid. MS-ESI: 420.2 [M+1].

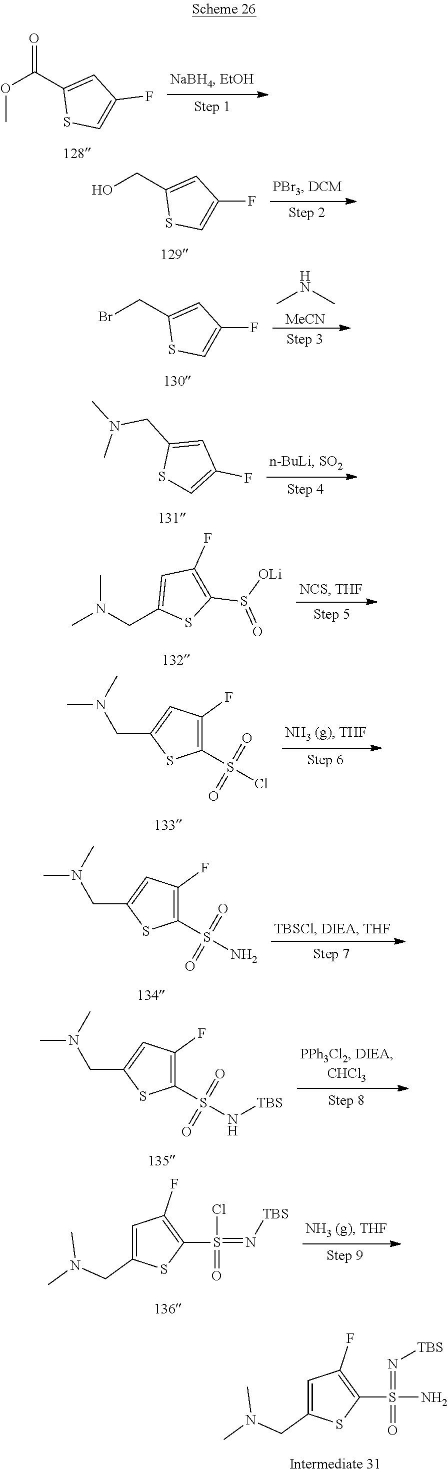

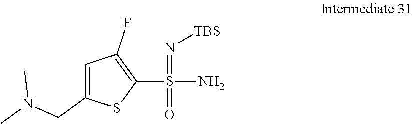

### N′-(tert-butyldimethylsilyl)-5-((dimethylamino)methyl)-3-fluorothiophene-2-sulfonimidamide

### Step 1: (4-Fluorothiophene-2-yl)methanol

Into a 1000-mL 3-necked round-bottom flask purged and maintained with an inert atmosphere of nitrogen, was placed methyl 4-fluorothiophene-2-carboxylate (10 g, 62.4 mmol) in EtOH (300 mL). To the above solution was added NaBH₄ (4.74 g, 124.8 mmol) with stirring at 0° C. The resulting solution was stirred for 30 min at 0° C. The resulting solution was allowed to react for an additional 16 h at RT. The reaction was then quenched by the addition of 10 mL of water. The resulting mixture was extracted with 3×1000 mL of ethyl acetate. Evaporation of combined ethyl acetate solution resulted in 6.4 g (77.5%) of the title compound as white oil.

### Step 2: 2-(Bromomethyl)-4-fluorothiophene

Into a 250-mL round-bottom flask, was placed (4-fluorothiophen-2-yl)methanol (8.5 g, 64.32 mmol) in DCM (70 mL). To the stirred solution was added PBr₃ (19.15 g, 70.75 mmol) at 0° C. The resulting solution was stirred for 30 min at 0′C, after which it was allowed to react for an additional 12 h at RT. The reaction was quenched with 20 mL of water and extracted with ethyl acetate 3×50 mL. The combined organic layer was dried over Na₂SO₄ and concentrated under vacuum. The residue was eluted from silica gel with ethyl acetate/petroleum ether (15/85). This resulted in 7.0 g (55.8%) of the title compound as yellow oil.

### Step 3: 1-(4-Fluorothiophen-2-yl)-N,N-dimethylmethanamine

Into a 250-mL round-bottom flask, was placed 2-(bromomethyl)-4-fluorothiophene (7.4 g, 37.9 mmol). To the solution was added dimethylamine in THF (2M, 37.9 mmol). The resulting solution was stirred for 16 h at RT. The reaction mixture was concentrated under vacuum. The residue was eluted from silica gel with ethyl acetate/petroleum ether (17/83). This resulted in 5.62 g (92.6%) of the title compound as a solid. MS-ESI: 160 [M+1].

### Step 4: Lithium 5-((dimethylamino)methyl)-3-fluorothiophene-2-sulfinate

Into a 250-mL 3-necked round-bottom flask purged and maintained with an inert atmosphere of nitrogen, was placed a solution of [(4-fluorothiophen-2-yl)methyl] dimethylamine (6.2 g, 38.9 mmol) in THF (60 mL), to the above solution was added n-BuLi (18.7 mL, 46.7 mmol, 2.5 M) dropwise at −78° C. in a liquid nitrogen/ethanol bath. The resulting solution was stirred for 30 min at −78° C. To the stirred solution, SO₂(g) (4.99 g, 78 mmol) was introduced in at −78° C. The resulting solution was allowed to react for an additional 120 min at RT. The resulting mixture was concentrated. This resulted in 10 g (crude) of the title compound as a yellow solid. MS-ESI: 228 [M−1].

### Step 5: 5-((Dimethylamino)methyl)-3-fluorothiophene-2-sulfonyl chloride

Into a 500-mL round-bottom flask, was placed a solution of 5-[(dimethylamino)methyl]-3-fluorothiophene-2-sulfinic acid (10 g, 44.7 mmol) in THF (100 mL), to the above solution was added NCS (7.18 g, 54 mmol) at 0° C. The resulting solution was stirred for 30 min at 0˜C. The resulting solution was allowed to react for an additional 100 min at RT. The reaction solution was used for next step without any purification.

### Step 6: 5-((Dimethylamino)methyl)-3-fluorothiophene-2-sulfonamide

Into a 500-mL round-bottom flask, was placed a solution of 5-[(dimethylamino)methyl]-3-fluorothiophene-2-sulfonyl chloride (10 g, 38.8 mmol) in THF (100 mL). To the above NH₃ (g) was introduced for 15 min at 0° C. The resulting solution was allowed to react for an additional 100 min at RT. Then the reaction solution was concentrated. The residue was eluted from silica gel with ethyl acetate/petroleum ether (60/40). This resulted in 2.1 g (22.7%) of the title compound as yellow oil. MS-ESI: 239 [M+1].

### Step 7: N-(tert-butyldimethylsilyl)-5-((dimethylamino)methyl)-3-fluorothiophene-2-sulfonamide

Into a 100-mL round-bottom flask purged and maintained with an inert atmosphere of nitrogen, was placed a solution of 5-[(dimethylamino)methyl]-3-fluorothiophene-2-sulfonamide (1.8 g, 7.55 mmol) in THF (30 mL). To the above solution was added NaH (60% wt. oil dispersion, 600 mg, 15 mmol) with stirring at 0° C. The resulting solution was stirred for 30 min at 0′C. This was followed by the addition of TBSCl (1.37 g, 9.09 mmol) at 0° C. The resulting solution was allowed to react for an additional 15 h at RT. The reaction solution was concentrated. The residue was eluted from silica gel with ethyl acetate. This resulted in 2 g (75.1%) of the title compound as yellow oil. MS-ESI: 353 [M+1].

### Step 8-1: N-(tert-butyldimethylsilyl)-5-((dimethylamino)methyl)-3-fluorothiophene-2-sulfonimidoyl chloride

Into a 500-mL 3-necked round-bottom flask purged and maintained with an inert atmosphere of nitrogen, was placed a solution of PPhCl₂ (59.2 g, 178 mmol) in CHCl₃ (100 mL). This was followed by the addition of DIEA (45.9 g, 355 mmol) dropwise with stirring at 0′C. The resulting solution was stirred for 15 min at RT. To this was added a solution of N-(tert-butyldimethylsilyl)-5-((dimethylamino)methyl)-3-fluorothiophene-2-sulfonamide (15.6 g, 44.4 mmol) in CHCl₃ (30 mL) dropwise with stirring at 0′C. The resulting solution was stirred for 30 min at 0° C. The reaction solution was used in the next step with no workup.

### Step 8-2: N′-(tert-butyldimethylsilyl)-5-((dimethylamino)methyl)-3-fluorothiophene-2-sulfonimidamide

Into a 250-mL 3-necked round-bottom flask, was placed N-(tert-butyldimethylsilyl)-5-((dimethylamino)methyl)-3-fluorothiophene-2-sulfonimidoyl chloride (2.8 g, 0.27 mmol) in CHCl₃ (20 mL). To the above NH₃(g) was introduced for 15 min at 0° C. The resulting solution was stirred for 15 min at 0° C. The resulting solution was stirred for 1 h at RT. The resulting mixture was diluted with 50 mL of H₂O. The resulting solution was extracted with 2×75 mL of DCM, the combined organic layer was dried over anhydrous sodium sulfate and concentrated. The residue was eluted from silica gel with ethyl acetate/petroleum ether (1:6). This resulted in 250 mg (9.4%) of the title compound as a yellow solid. MS-ESI: 352 [M+1].

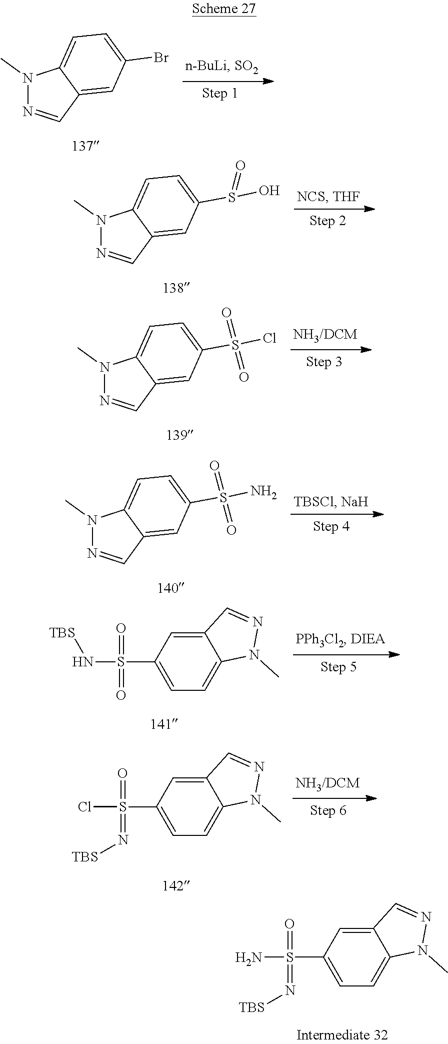

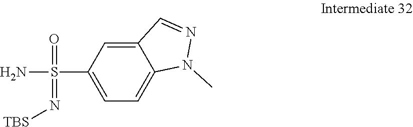

### Step 1: 1-Methyl-1H-indazole-5-sulfinic acid

Into a 100-mL 3-necked round-bottom flask purged and maintained with an inert atmosphere of nitrogen, was placed 5-bromo-1-methyl-1H-indazole (700 mg, 3.32 mmol) in THF (5 mL). To the above solution was added n-BuLi (1.6 mL, 3.98 mmol, 2.5 M) dropwise at −78° C. in a liquid nitrogen/ethanol bath. Then the solution was stirred for 30 min at −78′C. To the stirred solution, SO₂ (g) was introduced at −78° C. for 15 min. The resulting solution was allowed to react for an additional 120 min at RT. The resulting mixture was concentrated. This resulted 500 mg (76.8%) of the title compound as a yellow solid.

Steps 2-6 used similar procedures for converting compound 132″ to Intermediate 31 shown in Scheme 26 to afford Intermediate 32 from compound 138″. MS-ESI: 325 (M+1).

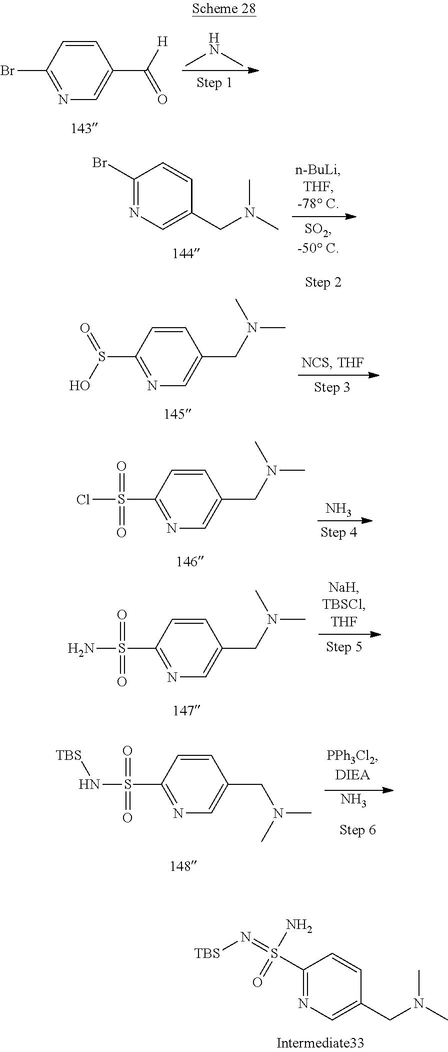

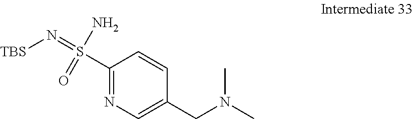

### N′-(tert-butyldimethylsilyl)-5-((dimethylamino)methyl)pyridine-2-sulfonimidamide

### Step 1: 1-(6-Bromopyridin-3-yl)-N,N-dimethylmethanamine

Into a 500 mL round-bottom flask, were added Ti(OEt)₄ (12.2 g, 53.7 mmol) and dimethylamine (4.85 g, 107 mmol) in methanol (50 mL) at RT. To this stirred solution was added 6-bromopyridine-3-carbaldehyde (5 g, 26.9 mmol) in methanol (30 mL) dropwise at 0° C. After stirring at RT for 3h, NaBH₄ (1.02 g, 26.9 mmol) was added and the resulting mixture was stirred overnight. The reaction was quenched by the addition of water/ice (30 mL) at 0° C. The resulting mixture was extracted with ethyl acetate (3×50 mL). The combined organic layers were dried over anhydrous Na₂SO₄. After filtration, the filtrate was concentrated under reduced pressure. The residue was purified by silica gel chromatography, eluted with ethyl acetate/petroleum ether (5:1) to afford the title compound (3.5 g, 60.5%) as yellow oil. MS-ESI: 215 (M+1).

### Step 2: 5-((Dimethylamino)methyl)pyridine-2-sulfinic acid

Into a 250-mL 3-necked round-bottom flask purged and maintained with an inert atmosphere of nitrogen, was placed [(6-bromopyridin-3-yl)methyl]dimethylamine (3.5 g, 16.27 mmol) in THF (30 mL). This was followed by the addition of n-BuLi (7.2 mL, 17.9 mmol, 2.5 M) dropwise with stirring at −78° C. in 30 min. To this was bubbled SO₂ at −78° C. for 15 min. The resulting solution was stirred for 1 h at −78′C. The resulting mixture was concentrated under vacuum. The crude product the title compound (4.0 g) was used in the next step directly without further purification.

### Step 3: 5-((Dimethylamino)methyl)pyridine-2-sulfonyl chloride

Into a 250 mL round-bottom flask, was placed 5-[(dimethylamino)methyl]pyridine-2-sulfinic acid (4.0 g crude) and THF (25 mL) at RT. To a stirred solution was added NCS (4 g, 0.03 mmol) in portions at 0′C. The resulting solution was stirred for 1.5 h at RT. The resulting mixture was used in the next step with no workup.

### Step 4: 5-((Dimethylamino)methyl)pyridine-2-sulfonamide

Into a 250 mL round-bottom flask, were added 5-[(dimethylamino)methyl]pyridine-2-sulfonyl chloride (crude from previous step) at RT. To this was bubbled NH₃ (g) for 10 min at 0° C. The resulting mixture was stirred for 1 h at 0′C. The residue was purified by reverse-phase flash chromatography with the following conditions (column, Cis silica gel; mobile phase, MeCN in water, 10% to 50% gradient in 10 min; detector, UV 254 nm.) to afford the title compound (1.2 g, 32.7%) as a yellow solid. MS-ESI: 216 [M+1]

### Step 5: N-(tert-butyldimethylsilyl)-5-((dimethylamino)methyl)pyridine-2-sulfonamide

Into a 100 mL round-bottom flask, were added 5-[(dimethylamino)methyl]pyridine-2-sulfonamide (700 mg, 3.25 mmol) in THF (15 mL) at 0° C. To this stirred solution was added NaH (60% wt. oil dispersion, 260 mg, 6.5 mmol) in portions at 0° C. under nitrogen atmosphere. The resulting mixture was stirred for 15 min at 0° C. under nitrogen atmosphere. Then TBSCl (980 mg, 6.5 mmol) was added to the above reaction mixture. After the addition was complete, the resulting mixture was stirred for 2 h at RT. The reaction was quenched by the addition of water/ice (10 mL) at 0° C. The resulting mixture was extracted with ethyl acetate (3×20 mL). The combined organic layers were dried over anhydrous Na₂SO₄. After filtration, the filtrate was concentrated under reduced pressure. The crude title compound (1.15 g) was used in the next step directly without further purification. MS-ESI: 330 [M+1].

### Step 6: N′-(tert-butyldimethylsilyl)-5-((dimethylamino)methyl)pyridine-2-sulfonimidamide

Into a 250 mL 3-necked round-bottom flask, was added PPh₃Cl₂ (5.89 g, 13.9 mmol) in CHCl₃ (18 mL). To this stirred solution was added DIEA (3.61 g, 27.9 mmol) dropwise at 0° C. under nitrogen atmosphere. The resulting mixture was stirred for 15 min at 0° C. under nitrogen atmosphere. Then N-(tert-butyldimethylsilyl)-5-[(dimethylamino)methyl]pyridine-2-sulfonamide (1.15 g, 3.49 mmol) in CHCl₃ (3 mL) was added to the above resulting mixture dropwise at 0° C. under nitrogen atmosphere. After the addition was complete the resulting mixture was stirred for 30 min. Then NH₃ (g) in DCM (40 mL) was added to the resulting mixture. The resulting mixture was stirred overnight. The resulting mixture was filtered, the filter cake was washed with ethyl acetate (3×20 mL). The filtrate was concentrated under reduced pressure. The residue was purified by Prep-TLC (ethyl acetate/methanol 100:1) to afford the title compound (600 mg, 52.3%) as a yellow solid. MS-ESI: 329 (M+1).

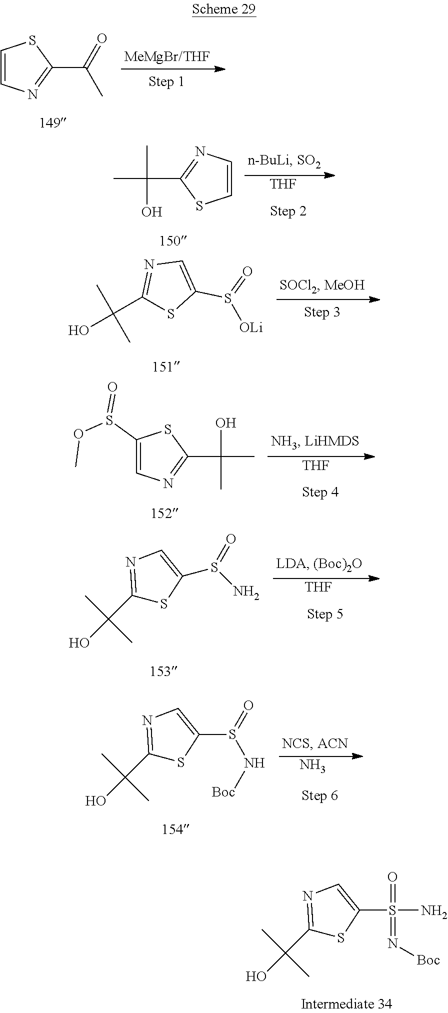

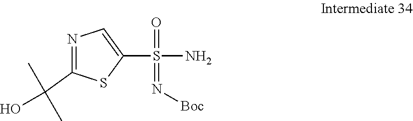 [SMILES: CC(C)(C)OC(=O)N=S(N)(=O)c1cnc(C(C)(C)O)s1]

### N′-(tert-butoxycarbonyl)-2-(2-hydroxypropan-2-yl)thiazole-5-sulfonimidamide

### Step 1: 2-(Thiazol-2-yl)propan-2-ol

Into a 10-L 4-necked round-bottom flask purged with and maintained under nitrogen was placed a solution of 1-(thiazol-2-yl)ethanone (200 g, 1.6 mol) in THF (4 L). This was followed by the addition of MeMgBr (3 M in THF, 942 mL) dropwise with stirring at 0° C. The mixture was stirred at 0′C for 2 h. After warmed the mixture to RT, the solution was stirred for an additional 16 h. Then the reaction was quenched by the addition of 3 L of NH₄Cl (sat.). The resulting solution was extracted with 3×1 L of ethyl acetate. The organic layers were combined and dried over anhydrous Na₂SO₄, then concentrated under vacuum. The residue was applied onto a silica gel column and eluted with a gradient of ethyl acetate/petroleum ether (1:3 to 1:1). This resulted in 210 g (93%) of the title compound as brown oil. MS-ESI: 144.0 (M+1). ¹H NMR (400 MHz, DMSO-d₆) δ 7.68 (d, J=3.2 Hz, 1H), 7.54 (d, J=3.2 Hz, 1H), 5.94 (s, 1H), 1.51 (s, 6H).

### Step 2: Lithium 2-(2-hydroxypropan-2-yl)thiazole-5-sulfinate

Into a 10-L 4-necked round-bottom flask purged with and maintained under nitrogen, was placed a solution of 2-(thiazol-2-yl)propan-2-ol (50 g, 349 mmol) in THF (1.5 L). This was followed by the addition of n-BuLi (2.5 M in hexane, 350 mL) dropwise with stirring at −78° C. The mixture was stirred at −78° C. for 1 h. Then SO₂ was bubbled into the mixture for 15 min below −30° C. The mixture was stirred for an additional 1 h at RT and then was concentrated under vacuum. This resulted in 87 g (crude) of the title compound as a light yellow solid. The crude product was used directly in the next step.

### Step 3: Methyl 2-(2-hydroxypropan-2-yl)thiazole-5-sulfinate

Into a 2-L 3-necked round-bottom flask, lithium 2-(2-hydroxypropan-2-yl)thiazole-5-sulfinate (87 g, crude) was dissolved in anhydrous MeOH (500 mL). Then SOCl₂ (43 g, 360 mmol) was added to the mixture dropwise with stirring at 0′C. The mixture was stirred overnight at RT and then was concentrated under vacuum. The residue was diluted with 500 mL of ethyl acetate. The resulting solution was washed with 2×200 mL of water and 2×200 mL of brine. The organic phase was dried over anhydrous Na₂SO₄, then concentrated under vacuum. This resulted in 72 g (crude) title compound as light yellow oil. The crude product was used directly in the next step. MS-ESI: 222[M+1]. ¹H NMR (400 MHz, DMSO-d₆) δ 8.15 (s, 1H), 6.32 (s, 1H), 3.65 (s, 3H), 1.53 (d, J=2.0 Hz, 6H).

### Step 4: 2-(2-Hydroxypropan-2-yl)thiazole-5-sulfinamide

Into a 10-L 4-necked round-bottom flask purged with and maintained under nitrogen, was placed a solution of methyl 2-(2-hydroxypropan-2-yl)thiazole-5-sulfinate (72 g, 326 mmol) in THF (500 mL). Then to the above NH₃ (0.5 M in THF, 2.0 L) was added. After cooling to-78° C., LiHMDS (1 M in THF, 2.0 L) was added to the mixture dropwise with stirring. Then the mixture was stirred at −78° C. for 2 h. The reaction was quenched by the addition of 500 mL of NH₄Cl (sat.). The resulting solution was extracted with 3×300 mL of ethyl acetate. The organic layers were combined, dried over anhydrous Na₂SO₄, then concentrated under vacuum. This resulted in 32 g (crude) title compound as brown oil. The crude product was used directly in the next step. MS-ESI: 207 [M+1]. ¹H NMR (400 MHz, DMSO-d₆) δ 7.77 (s, 1H), 6.73 (s, 2H), 6.17 (s, 1H), 1.51 (d, J=1.4 Hz, 6H).

### Step 5: Tert-butyl 2-(2-hydroxypropan-2-yl)thiazol-5-ylsulfinylcarbamate

Into a 1-L 3-necked round-bottom flask purged with and maintained under nitrogen, was placed a solution of 2-(2-hydroxypropan-2-yl)thiazole-5-sulfinamide (32 g, crude) in THF (300 mL). This was followed by the addition of LDA (2 M in THF, 116 mL) dropwise with string at 0° C. The mixture was stirred at 0° C. for 1 h, then (Boc)₂O (33.8 g, 155 mmol) was added in portions at 0° C. The mixture was warmed to RT and stirred for an additional 2 h. The reaction was quenched with 200 mL of ice-water (200 mL), and the pH value of the solution was adjusted to 6 with HCOOH. The resulting solution was extracted with 3×200 mL of ethyl acetate. The organic layers were combined, dried over anhydrous Na₂SO₄, and then concentrated under vacuum. The residue was eluted from silica gel with a gradient of ethyl acetate/petroleum ether (1:2 to 1:1). This resulted in 19 g (18%, 4 steps) title compound as a white solid. MS-ESI: 307 [M+1].

### Step 6: N-(tert-butyldimethylsilyl)-2-(2-hydroxypropan-2-yl)thiazole-5-sulfonimidamide

Into a 1-L 3-necked round-bottom flask purged with and maintained under nitrogen, tert-butyl 2-(2-hydroxypropan-2-yl)thiazol-5-ylsulfinylcarbamate (19 g, 62 mmol) was dissolved in freshly distilled ACN (200 mL). Then to the above solution was added NCS (9.8 g, 74 mmol) in portions. The mixture was stirred for 1 h at RT and then NH₃ was bubbled in the mixture for 15 min. The mixture was stirred at RT for 2 h and then was concentrated under vacuum. The residue was applied onto a silica gel column and eluted with a gradient of ethyl acetate/petroleum ether (1:2 to 1:1). This resulted in 13 g (65%) of the title compound as a white solid. MS-ESI: 322 [M+1]. ¹H NMR (300 MHz, DMSO-d₆) δ 7.99 (s, 1H), 7.72 (s, 2H), 6.29 (s, 1H), 1.49 (d, J=2.0 Hz, 6H), 1.27 (s, 9H).

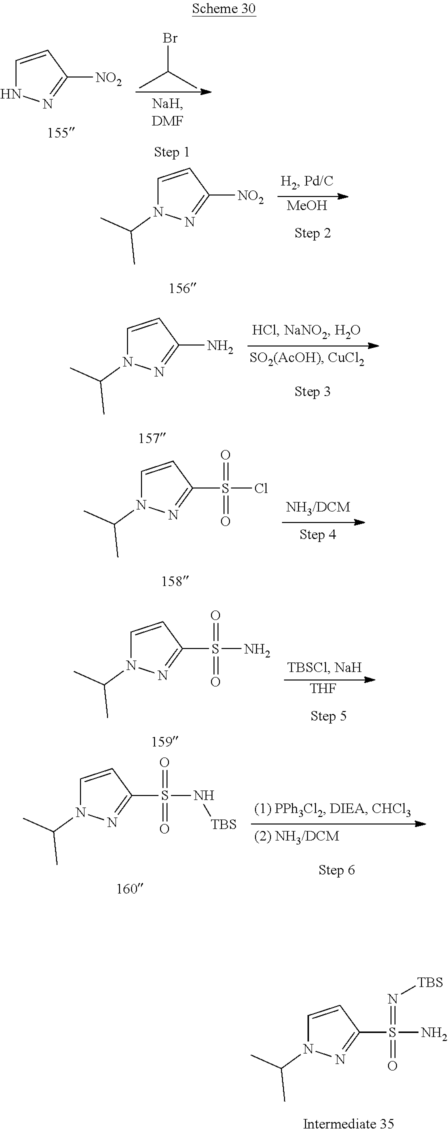

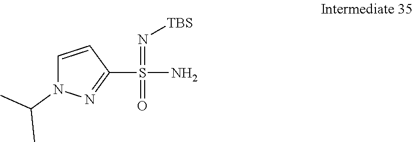

### N′-(tert-butyldimethylsilyl)-1-isopropyl-1H-pyrazole-3-sulfonimidamide

### Step 1: 1-Isopropyl-3-nitro-1H-pyrazole

Into a 250-mL round-bottom flask, was placed a solution of 3-nitro-1H-pyrazole (10 g, 88.4 mmol) in DMF (100 mL). This was followed by the addition of NaH (60% wt. oil dispersion, 3.9 g, 97.5 mmol) in portions at 0° C. The resulting solution was stirred for 0.5 h at 0° C. This was followed by the addition of 2-bromopropane (14.1 g, 114.6 mmol) dropwise with stirring at 0° C. in 10 min. The resulting solution was stirred for 16 h at RT and then was quenched by the addition of 100 mL of water. The resulting solution was extracted with 3×100 mL of ethyl acetate. The organic layers were combined and dried over anhydrous Na₂SO₄, and then concentrated under vacuum. The residue was eluted from silica gel and eluted with a gradient of ethyl acetate/petroleum ether (1:5 to 1:3). This resulted in 11.8 g (86%) of the title compound as yellow oil. MS-ESI: 156.1 (M+1).

### Step 2: 3-Amino-1-(propan-2-yl)-1H-pyrazole

Into a 250-mL round-bottom flask, was placed a solution of 1-isopropyl-3-nitro-1H-pyrazole (10.8 g, 69.6 mmol) in MeOH (100 mL). Then Pd/C (10% wt., 1.5 g) was added. The flask was evacuated and flushed three times with hydrogen. The mixture was stirred for 24 h at RT under an atmosphere of hydrogen. The solids were filtered out. The resulting filtrate was concentrated under vacuum. This resulted in 7.27 g (83%) of the title compound as yellow oil. MS-ESI: 126.1 (M+1).

### Step 3: 1-isopropyl-1H-pyrazole-3-sulfonyl chloride

Into a 1 L round-bottom flask, was placed a solution of 3-amino-1-(propan-2-yl)-1H-pyrazole (10 g, 80 mmol) in aq. HCl (6 N, 200 mL). This was followed by the addition of a solution of NaNO₂ (8.28 g, 120 mmol) in water (20 mL) dropwise with stirring at 0° C. The resulting solution was stirred for 30 min at 0° C. The above mixture was added to a saturated solution of SO₂ in AcOH (200 mL) dropwise with stirring at 0′C. Then to the above was added CuCl₂ (10.8 g, 80.7 mmol). The resulting solution was stirred for 1 h at RT and was then quenched by the addition of 200 mL of water. The resulting solution was extracted with 3×200 mL of DCM. The organic layers were combined, dried over anhydrous Na₂SO₄ and concentrated under vacuum. This resulted in 10 g (59.8%) of the title compound as yellow oil. The product was used in the next step without further purification.

### Step 4: 1-isopropyl-1H-pyrazole-3-sulfonamide

Into a 1000 mL round bottom flask, was placed a solution of 1-isopropyl-1H-pyrazole-3-sulfonyl chloride (10 g, 47.8 mmol) in DCM (50 mL). This was followed by the addition of a saturated solution of ammonia in DCM (500 mL) in portions with stirring at 0° C. The resulting solution was stirred for 1 h at 0° C. The resulting solution was concentrated and the residue was purified with SiO₂-gel column and eluted with ethyl acetate/petroleum ether (1:2 to 1:1). This resulted in 8.13 g (90%) of the title compound as yellow solid. MS-ESI: 190 [M+1].

Steps 5-6 used similar procedures for converting compound 147″ to Intermediate 33 shown in Scheme 28 to afford compound intermediate 35 from compound 159″. MS-ESI: 303 (M+1).

Schemes for phenylacetic acids Intermediates: Schemes 31-47 illustrate the preparation of phenylacetic acid intermediates.

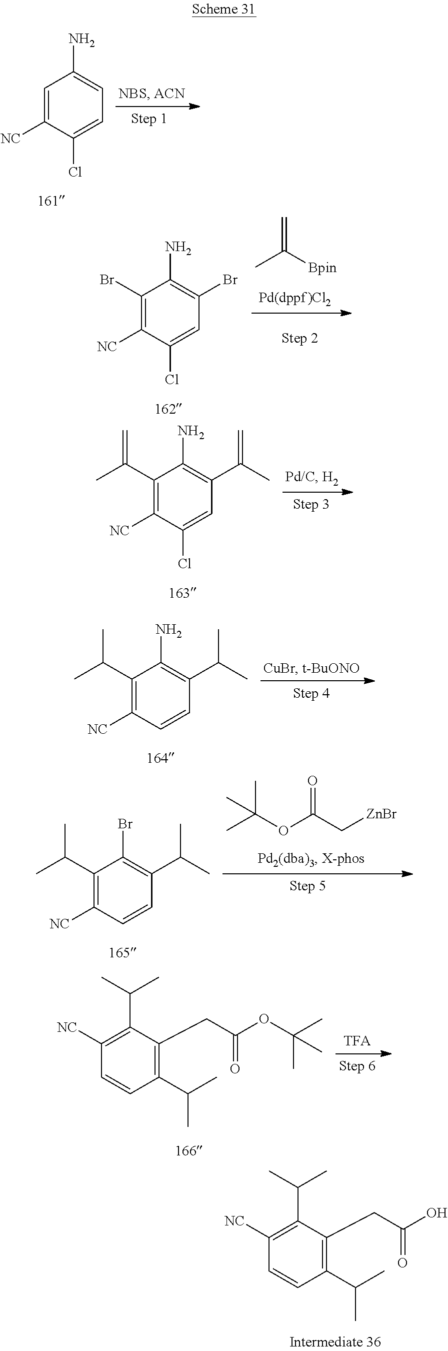

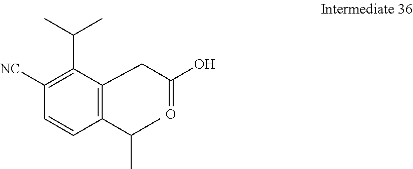 [SMILES: CC(C)c1ccc(C#N)c(C(C)C)c1CC(=O)O]

### 2-(3-Cyano-2,6-diisopropylphenyl)acetic acid

### Step 1: 3-Amino-2,4-dibromo-6-chlorobenzonitrile

Into a 500-mL round-bottom flask, was placed 5-amino-2-chlorobenzonitrile (10 g, 65.7 mmol) in ACN (200 mL). To the stirred solution was added NBS (29 g, 162 mmol) in portions. The resulting solution was stirred for 14 h at RT. The resulting mixture was concentrated. The residue was eluted from silica gel with ethyl acetate/petroleum ether (1:15 to 1:5). This resulted in 18 g of the title compound as a yellow solid. MS-ESI: 308/310 (M+1).

### Step 2: 3-Amino-6-chloro-2,4-di(prop-1-en-2-yl)benzonitrile

Into a 500-mL round-bottom flask purged and maintained with an inert atmosphere of nitrogen, was placed 3-amino-2,4-dibromo-6-chlorobenzonitrile (15 g, 48.0 mmol) in dioxane (200 mL) and H₂O (20 mL), 2-(tetramethyl-1,3,2-dioxaborolan-2-yl)prop-2-en-1-ylium (18.5 g, 111 mmol), Cs₂CO₃ (47 g, 144 mmol) and Pd(dppf)Cl₂ (1.5 g). The resulting solution was stirred for 14 h at 100° C. in an oil bath. The resulting mixture was concentrated. The residue was eluted from silica gel with ethyl acetate/petroleum ether (1:0 to 1:25). This resulted in 10 g of the title compound as brown oil. MS-ESI: 233 (M+1).

### Step 3: 3-Amino-2,4-diisopropylbenzonitrile

Into a 500-mL round-bottom flask purged and maintained with an inert atmosphere of nitrogen, was placed 3-amino-6-chloro-2,4-bis(prop-1-en-2-yl)benzonitrile (10 g, 43 mmol) in methanol (50 mL), to the stirred solution was added Pd/C (10% wt., 2 g). The flask was evacuated and filled three times with hydrogen. The resulting solution was stirred overnight at RT. The solids were filtered out. The resulting mixture was concentrated under vacuum. This resulted in 8 g of the title compound as brown oil. MS-ESI: 203 (M+1).

### Step 4: 3-Bromo-2,4-diisopropylbenzonitrile

Into a 250-mL round-bottom flask, was placed 3-amino-2,4-bis(propan-2-yl)benzonitrile (8 g, 39.5 mmol) in ACN (150 mL), to the stirred solution was added CuBr (11.3 g, 79.1 mmol) and tert-butyl nitrite (8.2 g, 79.1 mmol). The resulting solution was stirred for 3 h at 60° C. in an oil bath. The resulting mixture was concentrated. The residue was eluted from silica gel with ethyl acetate/petroleum ether (1:50). This resulted in 4.2 g (39.90%) of the title compound as purple oil. MS-ESI: 266/268[M+1]

### Step 5: Tert-butyl 2-(3-cyano-2,6-diisopropylphenyl)acetate

Into a 250-mL round-bottom flask purged and maintained with an inert atmosphere of nitrogen, was placed 3-bromo-2,4-bis(propan-2-yl)benzonitrile (3.1 g, 11.6 mmol) in THF (100 mL), to the stirred solution was added Xphos (555.2 mg, 1.16 mmol), Pd₂(dba)₃ (533.2 mg, 0.58 mmol) and tert-butyl 2-(bromozincio)acetate (7.6 g, 29.12 mmol). The resulting solution was stirred for 3 h at 65° C. in an oil bath. The resulting mixture was concentrated. The residue was eluted from silica gel with ethyl acetate/petroleum ether (1:50). This resulted in 3.0 g (85.5%) of the title compound as purple oil. MS-ESI: 302 [M+1].

### Step 6: 2-(3-Cyano-2,6-diisopropylphenyl)acetic acid

Into a 100-mL round-bottom flask, was placed tert-butyl 2-[3-cyano-2,6-bis(propan-2-yl)phenyl]acetate (3.4 g, 11.28 mmol) in DCM (15 mL), to the stirred solution was added TFA (15 mL). The resulting solution was stirred for 3 h at RT. The resulting mixture was concentrated. The residue was eluted from silica gel with ethyl acetate/petroleum ether (1:3). This resulted in 2.6 g (93.9%) of the title compound as a light yellow solid. MS-ESI: 246 [M+1].

TABLE 13
The Intermediates in the following Table were prepared using the similar procedures
for converting compound 161″ to Intermediate 36 shown in Scheme 31 from appropriated
starting materials.

| | | | Exact Mass |
|---|---|---|---|
| Intermediate # | Structure | IUPAC Name | [M − H]⁻ |
| Intermediate 37 | 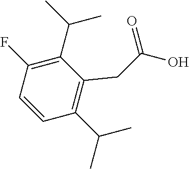 [SMILES: CC(C)c1ccc(F)c(C(C)C)c1CC(=O)O] | 2-(3-fluoro-2,6- diisopropylphenyl)acetic acid | 237 |
| Intermediate 38 | 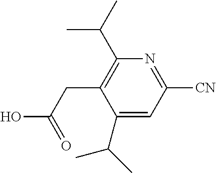 [SMILES: CC(C)c1cc(C#N)nc(C(C)C)c1CC(=O)O] | 2-(6-cyano-2,4-diisopropylpyridin- 3-yl)acetic acid | 247 |

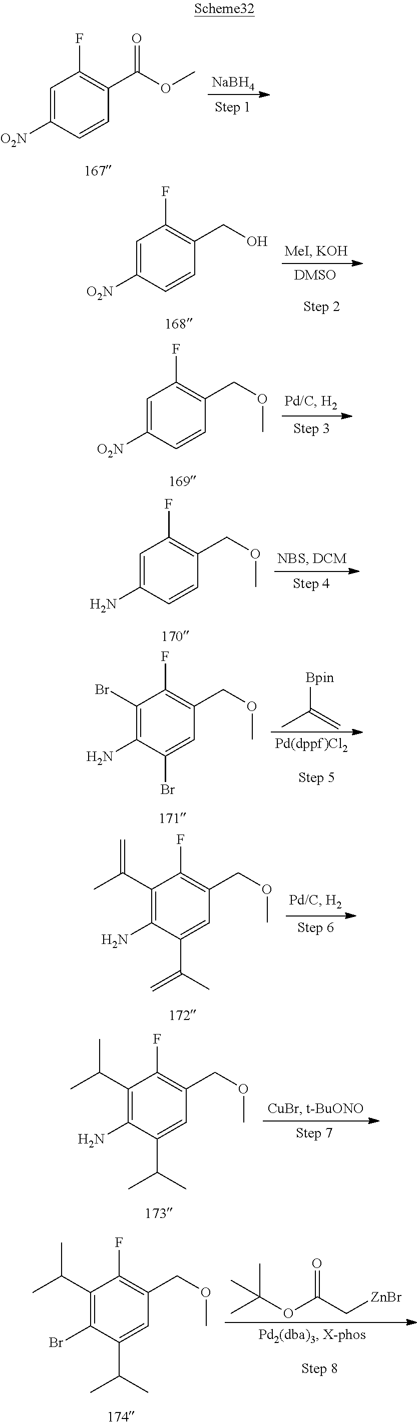

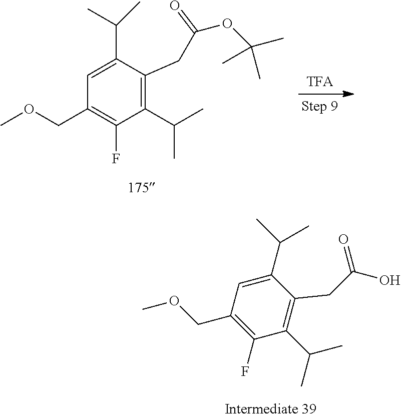

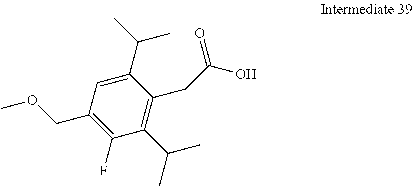 [SMILES: COCc1cc(C(C)C)c(CC(=O)O)c(C(C)C)c1F]

### 2-(3-Fluoro-2,6-diisopropyl-4-(methoxymethyl)phenyl)acetic acid

### Step 1: (2-Fluoro-4-nitrophenyl)methanol

Into a 500-mL round-bottom flask, was placed methyl 2-fluoro-4-nitrobenzoate (10 g, 50.2 mmol) in methanol (100 mL). This was followed by the addition of NaBH₄ (9.5 g, 251 mmol) in portions over 30 min. The resulting solution was stirred for 4 h at RT. The resulting solution was diluted with 400 mL of ethyl acetate. The resulting mixture was washed with 200 mL of water and 200 mL of brine. The organic layer was dried over anhydrous sodium sulfate and then concentrated. This resulted in 3.6 g of the title compound as an off white solid. MS-ESI: 172 (M+1).

### Step 2: 2-Fluoro-1-(methoxymethyl)-4-nitrobenzene

Into a 50-mL round-bottom flask, was placed (2-fluoro-4-nitrophenyl)methanol (3.6 g, 21.0 mmol) in DMSO (10 mL). To the stirred solution was added KOH (4.72 g, 84.2 mmol) in portions and MeI (11.9 g, 84.1 mmol) dropwise at RT. The resulting solution was stirred for overnight at RT. The reaction was then quenched by the addition of water. The resulting solution was extracted with 200 mL of dichloromethane. The organic layers were combined and washed with 200 mL of brine. Then the organic layer was dried over anhydrous sodium sulfate and concentrated. The residue was eluted from silica gel with ethyl acetate/petroleum ether (1:1) to give the title compound as 2.1 g yellow solid. MS-ESI: 186 (M+1).

### Step 3: 3-Fluoro-4-(methoxymethyl)aniline

Into a 250-mL round-bottom flask purged and maintained with an inert atmosphere of nitrogen, was placed 2-fluoro-1-(methoxymethyl)-4-nitrobenzene (2.4 g, 12.9 mmol) in methanol (50 mL), to the stirred solution was added Pd/C (10% wt. oil dispersion, 240 mg). The flask was evacuated and filled three times with hydrogen. The resulting solution was stirred overnight at RT. The solids were filtered out. The resulting mixture was concentrated under vacuum. The resulting mixture was concentrated to give the title compound as 2.4 g yellow solid. MS-ESI: 156 (M+1).

### Step 4: 2,6-Dibromo-3-fluoro-4-(methoxymethyl)aniline

Into a 100-mL round-bottom flask, was placed 3-fluoro-4-(methoxymethyl)aniline (1.7 g, 10.96 mmol) in DCM (50 mL). This was followed by the addition of NBS (4.3 g, 12.1 mmol) in portions. The resulting solution was stirred for 1 h at RT. The reaction was then quenched by the addition of water/ice. The resulting solution was extracted with 200 mL of ethyl acetate. The resulting mixture was washed with 200 mL of water and 200 mL of brine. The organic layer was dried over anhydrous sodium sulfate and concentrated. The residue was eluted from silica gel with ethyl acetate/petroleum ether (1:3) to give 4 g title compound as a yellow solid. MS-ESI: 311/313 (M+1).

### Step 5: 3-Fluoro-4-(methoxymethyl)-2,6-di(prop-1-en-2-yl)aniline

Into a 500-mL round-bottom flask purged and maintained with an inert atmosphere of nitrogen, was placed 2,6-dibromo-3-fluoro-4-(methoxymethyl)aniline (14 g, 44.7 mmol) in dioxane (200 mL) and H₂O (20 mL). To the stirred solution was added 4,4, 5, 5-tetramethyl-2-(prop-1-en-2-yl)-1,3,2-dioxaborolane (18.8 g, 111 mmol), Pd(dppf)Cl₂ (3.27 g, 4.47 mmol) and Cs₂CO₃ (29.2 g, 89.5 mmol). The resulting solution was stirred for 5 h at 65° C. in an oil bath. The reaction was then quenched by the addition of 10 mL of water. The resulting solution was extracted with 3×20 ml of ethyl acetate and dried over anhydrous sodium sulfate and concentrated. The residue was eluted from silica gel with ethyl acetate/petroleum ether (1:5). This resulted in 2.0 g (19.0%) of the title compound as yellow oil. MS-ESI: 236 (M+1).

### Step 6: 3-Fluoro-2,6-diisopropyl-4-(methoxymethyl)aniline

Into a 100-mL round-bottom flask, was placed 3-fluoro-4-(methoxymethyl)-2,6-bis(prop-1-en-2-yl) aniline (2.0 g, 8.50 mmol) in methanol (20 mL). To the stirred solution was added Pd/C (10% wt., 200 mg). The flask was evacuated and filled three times with hydrogen. The resulting solution was stirred 5h at RT. The solids were filtered out. The resulting filtrate was concentrated under vacuum. This resulted in 1.8 g (88.5%) of the title compound as yellow oil. MS-ESI: 240 (M+1).

### Step 7: 2-Bromo-4-fluoro-1,3-diisopropyl-5-(methoxymethyl)benzene

Into a 100-mL round-bottom flask purged and maintained with an inert atmosphere of nitrogen, was placed 3-fluoro-4-(methoxymethyl)-2,6-bis(propan-2-yl)aniline (1.0 g, 4.18 mmol) in CH₃CN (30 mL). To the above solution was added CuBr (2.4 g, 16.7 mmol) and t-BuONO (−1.72 g, 16.7 mmol) with stirring. The resulting solution was stirred for 3 h at 65° C. in an oil bath. The reaction was then quenched by the addition of 20 mL of water. The resulting solution was extracted with 3×20 ml of ethyl acetate and dried over anhydrous sodium sulfate and concentrated. The residue was eluted from silica gel with petroleum ether. This resulted in 500 mg (39.4%) of the title compound as a yellow solid. MS-ESI: 303/305 [M+1].

### Step 8: Tert-butyl 2-(3-fluoro-2,6-diisopropyl-4-(methoxymethyl)phenyl)acetate

Into a 100-mL round-bottom flask purged and maintained with an inert atmosphere of argon, was placed 2-bromo-4-fluoro-5-(methoxymethyl)-1,3-bis(propan-2-yl)benzene (1.0 g, 3.30 mmol) in THF (40 mL). To the stirred solution was added tert-butyl 2-(bromozincio)acetate (2.58 g, 9.89 mmol), Pd₂(dba)₃CHCl₃ (170 mg, 0.16 mmol), and Xphos (157 mg, 0.33 mmol). The resulting solution was stirred for 3 h at 65° C. in an oil bath. The reaction was then quenched by the addition of 20 mL of water. The resulting solution was extracted with 3×30 ml of DCM and dried over anhydrous sodium sulfate and concentrated. The residue was eluted from silica gel with ethyl acetate/petroleum ether (1:20). This resulted in 200 mg (17.9%) of the title compound as yellow oil. MS-ESI: 339 [M+1].

### Step 9: 2-(3-Fluoro-2,6-diisopropyl-4-(methoxymethyl)phenyl)acetic acid

Into a 50-ml round-bottom flask, was placed tert-butyl 3-fluoro-4-(methoxymethyl)-2,6-bis(propan-2-yl)benzoate (300 mg, 0.92 mmol) in DCM (6 mL), to the stirred solution was added TFA (2 mL). The resulting solution was stirred for 2 h at RT. The resulting mixture was concentrated. The residue was eluted from silica gel with DCM/methanol (1:20). This resulted in 170 mg of the title compound as yellow oil. MS-ESI: 281 (M−1).

TABLE 14
The Intermediates in the following Table were prepared using the similar procedures
for converting compound 167″ to Intermediate 39 shown in Scheme 32 from appropriated
starting materials.

| | | | Exact Mass |
|---|---|---|---|
| Intermediate # | Structure | IUPAC Name | [M − H]⁻ |
| Intermediate 40 | 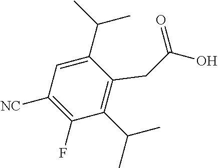 [SMILES: CC(C)c1cc(C#N)c(F)c(C(C)C)c1CC(=O)O] | 2-(4-cyano-3-fluoro-2,6- diisopropylphenyl)acetic acid | 262 |
| Intermediate 41 | 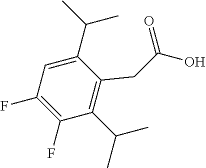 [SMILES: CC(C)c1cc(F)c(F)c(C(C)C)c1CC(=O)O] | 2-(3,4-difluoro-2,6- diisopropylphenyl)acetic acid | 255 |
| Intermediate 42 | 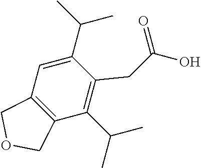 [SMILES: CC(C)c1cc2c(c(C(C)C)c1CC(=O)O)COC2] | 2-(4,6-diisopropyl-1,3- dihydroisobenzofuran-5-yl)acetic acid | 261 |
| Intermediate 43 | 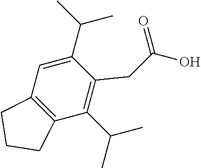 [SMILES: CC(C)c1cc2c(c(C(C)C)c1CC(=O)O)CCC2] | 2-(4,6-diisopropyl-2,3-dihydro-1H- inden-5-yl)acetic acid | 259 |

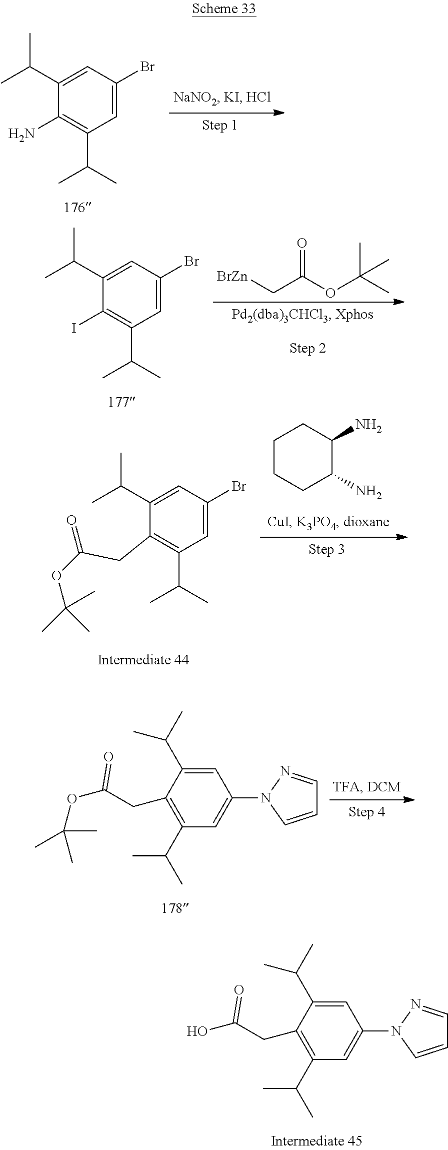

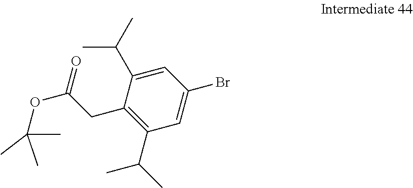 [SMILES: CC(C)c1cc(Br)cc(C(C)C)c1CC(=O)OC(C)(C)C]

### Teri-butyl 2-(4-bromo-2,6-diisopropylphenyl)acetate

### Step 1: 5-Bromo-2-iodo-1,3-diisopropylbenzene

Into a 500-mL round-bottom flask purged and maintained with an inert atmosphere of nitrogen, was placed 4-bromo-2,6-bis(propan-2-yl)aniline (5 g, 19.6 mmol) in HCl (6 M, 60 mL). This was followed by the addition of a solution of NaNO₂ (2.5 g, 36.3 mmol) in water (5 mL) dropwise with stirring at −10° C. The resulting solution was stirred for 30 min at −10° C. Then to the above was added KI (11 g, 66.3 mmol). The resulting solution was stirred for 1 h at RT. The reaction was then quenched by the addition of 100 mL of water. The resulting solution was extracted with 2×200 mL of ethyl acetate and the organic layers combined. The residue was eluted from silica gel with ethyl acetate/petroleum ether (0/1). This resulted in 5.95 g (83.0%) of the title compound as a brown liquid. MS-ESI: 366/368 (M+1).

### Step 2: Tert-butyl 2-(4-bromo-2,6-diisopropylphenyl)acetate

Into a 100-mL round-bottom flask purged and maintained with an inert atmosphere of nitrogen, was placed 5-bromo-2-iodo-1,3-bis(propan-2-yl)benzene (2.0 g, 5.45 mmol) in THF (50 mL). To the stirred solution was added Pd₂(dba)₃ (504 mg, 0.55 mmol), Xphos (262 mg, 0.55 mmol) and tert-butyl 2-(bromozincio)acetate (2.13 g, 8.66 mmol). The resulting solution was stirred for 30 min at RT. The resulting solution was allowed to react with stirring for an additional 3 h at 60° C. The resulting mixture was concentrated. The residue was eluted from silica gel with petroleum ether. This resulted in 360 mg (18.6%) of the title compound as a solid. MS-ESI: 355/357 (M+1).

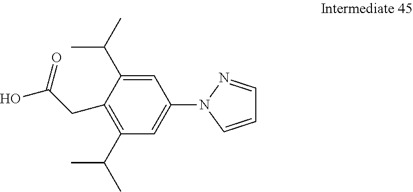 [SMILES: CC(C)c1cc(-n2cccn2)cc(C(C)C)c1CC(=O)O]

### 2-(2,6-diisopropyl-4-(1H-pyrazol-1-yl)phenyl)acetic acid

### Step 3: Tert-butyl 2-(2,6-diisopropyl-4-(1H-pyrazol-1-yl)phenyl)acetate

Into a 50-mL round-bottom flask purged and maintained with an inert atmosphere of nitrogen, was placed a mixture of tert-butyl 2-(4-bromo-2,6-diisopropylphenyl)acetate (360 mg, 1.01 mmol) in dioxane (10 mL). To the stirred solution was added 1H-pyrazole (275 mg, 4.04 mmol), copper(I) iodide (76 mg, 0.40 mmol) and potassium phosphate (642 mg, 3.03 mmol). To the above (1R, 2R)-cyclohexane-1,2-diamine (0.05 mL, 0.40 mmol) was added dropwise. The resulting solution was refluxed overnight. The reaction was then concentrated and the residue was eluted from silica gel with ethyl acetate/petroleum ether (1:20). This resulted in 120 mg (35%) of the title compound as a yellow oil. MS-ESI: 342.2 (M+1).

### Step 4: 2-(2,6-Diisopropyl-4-(1H-pyrazol-1-yl)phenyl)acetic acid

Into a 50-mL sealed tube purged and maintained with an inert atmosphere of nitrogen, was placed a solution of tert-butyl 2-(2,6-diisopropyl-4-(1H-pyrazol-1-yl)phenyl)acetate (120 mg, 0.35 mmol) in TFA (10 mL). The resulting solution was stirred overnight at RT. The reaction was then concentrated and used in the next step without purification. MS-ESI: 286.2 (M+1).

TABLE 15
The Intermediates in the following Table were prepared using the similar procedures
for converting compound 176″ to Intermediate 44 shown in Scheme 33 from appropriated
starting materials.

| | | | Exact Mass |
|---|---|---|---|
| Intermediate # | Structure | IUPAC Name | [M + H]⁺ |
| Intermediate 46 | 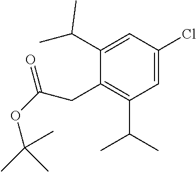 [SMILES: CC(C)c1cc(Cl)cc(C(C)C)c1CC(=O)OC(C)(C)C] | tert-butyl 2-(4-chloro-2,6- diisopropylphenyl)acetate | 311 |

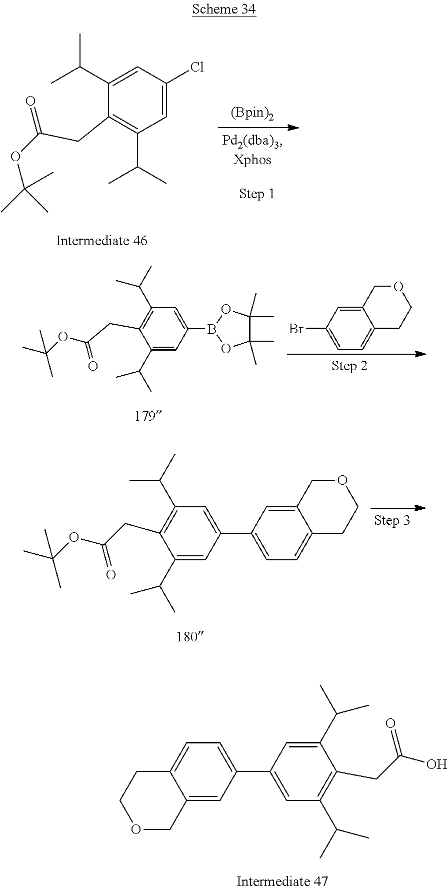

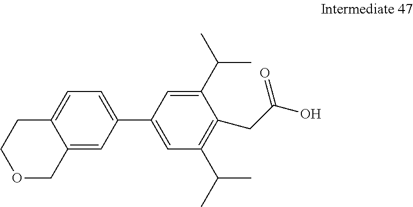 [SMILES: CC(C)c1cc(-c2ccc3c(c2)COCC3)cc(C(C)C)c1CC(=O)O]

### 2-(4-(Isochroman-7-yl)-2,6-diisopropylphenyl)acetic acid

### Step 1: Tert-butyl 2-(2,6-diisopropyl-4-(4,4, 5, 5-tetramethyl-1,3,2-dioxaborolan-2-yl)phenyl)acetate

Into a 50-mL round-bottom flask purged and maintained with an inert atmosphere of nitrogen, was placed tert-butyl 2-[4-chloro-2,6-bis(propan-2-yl)phenyl]acetate (310 mg, 1.00 mmol) in dioxane (10 mL). To the stirred solution was added 4,4,4′,4′,5,5,5′,5′-octamethyl-2,2′-bi(1,3,2-dioxaborolane) (508 mg, 2.0 mmol). KOAc (195 mg, 1.99 mmol), Xphos (95.1 mg, 0.20 mmol) Pd₂(dba)₃ (91.3 mg, 0.10 mmol). The resulting solution was stirred for 16 h at 90° C. in an oil bath under nitrogen. Then the mixture was concentrated and the residue was eluted from silica gel with ethyl acetate/petroleum ether (1:20). This resulted in 400 mg (99.7%) of the title compound as a crude solid. MS-ESI: 403 (M+1).

### Step 2: Tert-butyl 2-(4-(isochroman-7-yl)-2,6-diisopropylphenyl)acetate

Into a 50-mL round-bottom flask purged and maintained with an inert atmosphere of nitrogen, was placed tert-butyl 2-(2,6-diisopropyl-4-(4,4,5,5-tetramethyl-1,3,2-dioxaborolan-2-yl)phenyl)acetate (402 mg, 1.00 mmol) in dioxane (10 mL) and H₂O (2.5 mL). To the stirred solution was added Cs₂CO₃ (652.0 mg, 2.00 mmol), 7-bromo-3,4-dihydro-1H-2-benzopyran (212.9 mg, 1.00 mmol) and Pd(dppf)Cl₂ (73.1 mg, 0.10 mmol,). The resulting solution was stirred for 4 h at 80′C in an oil bath. The resulting solution was diluted with 20 mL of ethyl acetate. The resulting mixture was washed with 2×20 mL of H₂O and 2×20 mL of brine. The residue was eluted from silica gel with ethyl acetate/petroleum ether (1:10). This resulted in 300 mg (73.4%) of the title compound as a light brown solid. MS-ESI: 409 (M+1).

### Step 3: 2-(4-(Isochroman-7-yl)-2,6-diisopropylphenyl)acetic acid

Into a 25-mL round-bottom flask purged and maintained with an inert atmosphere of nitrogen, was placed tert-butyl 2-[4-(3,4-dihydro-1H-2-benzopyran-7-yl)-2,6-bis(propan-2-yl)phenyl]acetate (300 mg, 0.73 mmol) in DCM (4 mL) and TFA (1 mL). The resulting solution was stirred for 16 h at RT. The resulting mixture was concentrated. The residue was eluted from silica gel with ethyl acetate/petroleum ether (1:2). This resulted in 80 mg (30.9%) of the title compound as a light brown solid. MS-ESI: 351 (M−1).

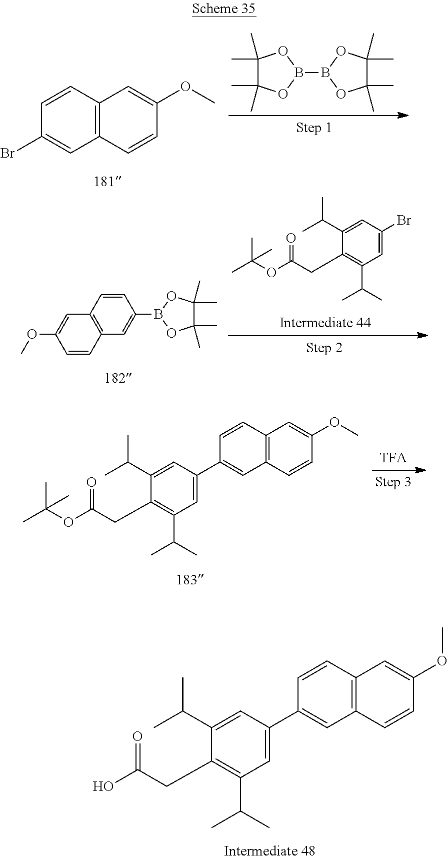

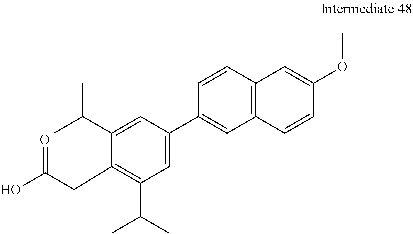 [SMILES: COc1ccc2cc(-c3cc(C(C)C)c(CC(=O)O)c(C(C)C)c3)ccc2c1]

### 2-(2,6-Diisopropyl-4-(6-methoxynaphthalen-2-yl)phenyl)acetic acid

### Step 1: 2-(6-Methoxynaphthalen-2-yl)-4,4, 5, 5-tetramethyl-1,3,2-dioxaborolane

Into a 50-mL round-bottom flask, was placed 2-bromo-6-methoxynaphthalene (115 mg, 0.49 mmol) in dioxane (5 mL), to the stirred solution was added potassium acetate (175 mg, 1.27 mmol), 4,4, 5, 5-tetramethyl-2-(4,4, 5, 5-tetramethyl-1,3,2-dioxaborolan-2-yl)-1,3,2-dioxaborolane (113 mg, 0.45 mmol) and Pd(PPh₃)₂Cl₂ (9 mg). The resulting solution was stirred for 10 h at 110° C. The resulting solution was extracted with 3×20 mL of ethyl acetate and dried over anhydrous sodium sulfate. The solids were filtered out. The resulting mixture was concentrated under vacuum. This resulted in 120 mg of the title compound as an off-white solid. MS-ESI: 285 (M+1).

### Step 2: 2-(2,6-Diisopropyl-4-(6-methoxynaphthalen-2-yl)phenyl)acetic acid

Into a 50-mL round-bottom flask, was placed 2-(6-methoxynaphthalen-2-yl)-4,4, 5, 5-tetramethyl-1,3,2-dioxaborolane (100 mg) in dioxane (15 mL) and H₂O (1.5 mL), to the stirred solution was added Cs₂CO₃ (344 mg), Pd(dppf)Cl₂ (27.5 mg), 2-[4-bromo-2,6-bis(propan-2-yl)phenyl] acetic acid (125 mg). The resulting solution was stirred for 15 h at 80′C. The resulting mixture was concentrated under vacuum. The residue was eluted from silica gel with ethyl acetate/petroleum ether (1:3). This resulted in 90 mg (58.9%) of the title compound as a yellow solid. MS-ESI: 433 (M+1).

### Step 3: 2-(2,6-Diisopropyl-4-(6-methoxynaphthalen-2-yl)phenyl)acetic acid

Into a 50-mL round-bottom flask, was placed tert-butyl 2-[4-(6-methoxynaphthalen-2-yl)-2,6-bis(propan-2-yl)phenyl]acetate (80 mg, 0.18 mmol) in DCM (5 mL) and TFA (2.0 mL). The resulting solution was stirred for 1 h at RT. The resulting mixture was concentrated. This resulted in 80 mg (crude) of the title compound as a light yellow solid. MS-ESI: 377 [M+1]

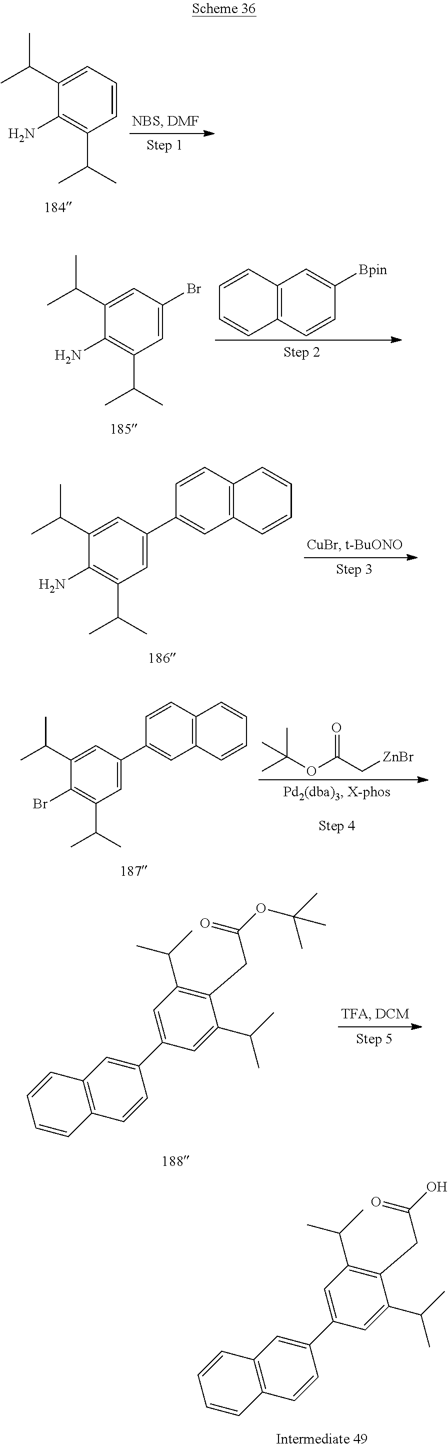

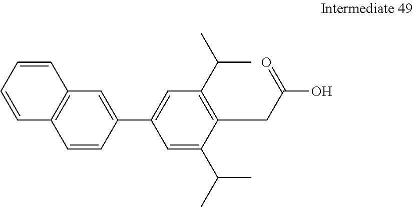 [SMILES: CC(C)c1cc(-c2ccc3ccccc3c2)cc(C(C)C)c1CC(=O)O]

### 2-(2,6-Diisopropyl-4-(naphthalen-2-yl)phenylacetic acid

### Step 1: 4-Bromo-2,6-diisopropylaniline

Into a 250-mL 3-necked round-bottom flask purged and maintained with an inert atmosphere of nitrogen, was placed 2,6-bis(propan-2-yl)aniline (20 g, 56.4 mmol) in DMF (200 mL), to the stirred solution was added NBS (20.1 g, 112 mmol). The resulting solution was stirred for 6 h at RT. The resulting mixture was washed with 100 ml of water. The resulting solution was extracted with 3×100 ml of ethyl acetate dried over anhydrous sodium sulfate. The solids were filtered out. The resulting mixture was concentrated. The residue was eluted from silica gel with ethyl acetate/petroleum ether (1:1). This resulted in 16 g (55.3%) of the title compound as a white solid. MS-ESI: 256/258 (M+1).

### Step 2: 2,6-Biisopropyl-4-(naphthalen-2-yl)aniline

Into a 500-mL round-bottom flask purged and maintained with an inert atmosphere of nitrogen, was placed a solution of 4-bromo-2,6-bis(propan-2-yl)aniline (10 g, 39.0 mmol) in dioxane (250 mL) and H₂O (25 mL). To the stirred solution was added 4,4, 5, 5-tetramethyl-2-(naphthalen-2-yl)-1,3,2-dioxaborolane (11.9 g, 46.8 mmol), Pd(dppf)Cl₂ (7.81 g, 7.8 mmol) and Cs₂CO₃ (25.4 g, 78.1 mmol). The resulting solution was stirred for 10 min at RT. The resulting solution was then allowed to react for an additional 19 h at 80° C. The resulting mixture was concentrated. The residue was eluted from silica gel with ethyl acetate/petroleum ether (1:1). This resulted in 6.5 g (54.9%) of the title compound as a red solid. MS-ESI: 304 (M+1).

### Step 3: 2-(4-Bromo-3, 5-diisopropylphenyl)naphthalene

Into a 250-mL round-bottom flask purged and maintained with an inert atmosphere of nitrogen, was placed a solution of 4-(naphthalen-2-yl)-2,6-bis(propan-2-yl)aniline (6.0 g, 19.8 mmol) in ACN (100 mL). To the stirred solution was added tert-butyl nitrite (4.08 g, 39.5 mmol) and CuBr (5.67 g, 39.5 mmol). The resulting solution was stirred for 30 min at RT. The resulting solution was allowed to react with stirring for an additional 180 min at 60˜C. The mixture was concentrated and the residue was eluted from silica gel with PE. This resulted in 105 mg (17.3%) of the title compound as a red solid. MS-ESI: 367/369 (M+1).

### Step 4: Tert-butyl 2-(2,6-diisopropyl-4-(naphthalen-2-yl)phenyl)acetate

Into a 100-mL round-bottom flask purged and maintained with an inert atmosphere of nitrogen, was placed a solution of 2-[4-bromo-3, 5-bis(propan-2-yl)phenyl]naphthalene (2 g, 5.44 mmol) in THF (50 mL), to the above solution was added XPhos (0.3 g, 0.54 mmol), and Pd₂(dba)₃CH₂Cl₂ (0.2 g, 0.27 mmol). The resulted solution was stirred for 15 min at RT. Then to the mixture was added tert-butyl 2-(bromozincio)acetate (2.8 g, 10.9 mmol) with stirring. The resulting solution was allowed to react for an additional 180 min at 65° C. The residue was eluted from silica gel with PE. This resulted in 1.0 g (45.6%) of the title compound as a yellow solid. MS-ESI: 403 (M+1).

### Step 5: 2-(2,6-Diisopropyl-4-(naphthalen-2-yl)phenyl)acetic acid

Into a 250-mL round-bottom flask, was placed tert-butyl 2-[4-(naphthalen-2-yl)-2,6-bis(propan-2-yl)phenyl]acetate (2.48 g, 6.16 mmol) in TFA (20 mL) and DCM (20 mL). The resulting solution was stirred for 5 h at RT. Then the mixture was concentrated. The residue was eluted from silica gel with ethyl acetate/petroleum ether (13/100). This resulted in 1.68 g (78.5%) of the title compound as a yellow solid. MS-ESI: 347 (M+1).

TABLE 16
The Intermediates in the following Table were prepared using the similar procedures for converting compound 184″ to Intermediate
49 shown in Scheme 36 from appropriated starting materials.

| Intermediate | | | Exact Mass |
|---|---|---|---|
| # | Structure | IUPAC Name | [M − H]⁻ |
| Intermediate 50 | 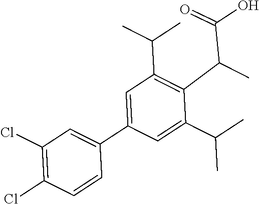 [SMILES: CC(C)c1cc(-c2ccc(Cl)c(Cl)c2)cc(C(C)C)c1C(C)C(=O)O] | 2-(3′,4′-dichloro-3,5-diisopropyl- [1,1′-biphenyl]-4-yl)acetic acid | 363 |
| Intermediate 51 | 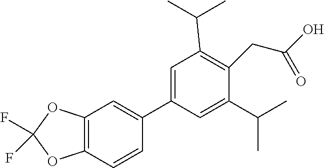 [SMILES: CC(C)c1cc(-c2ccc3c(c2)OC(F)(F)O3)cc(C(C)C)c1CC(=O)O] | 2-(4-(2,2- difluorobenzo[d][1,3]dioxol-5-yl)- 2,6-diisopropylphenyl)acetic acid | 375 |
| Intermediate 52 | 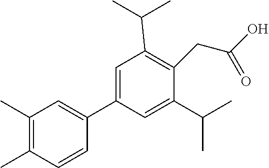 [SMILES: Cc1ccc(-c2cc(C(C)C)c(CC(=O)O)c(C(C)C)c2)cc1C] | 2-(3,5-diisopropyl-3′,4′-dimethyl- [1,1′-biphenyl]-4-yl)acetic acid | 323 |
| Intermediate 53 | 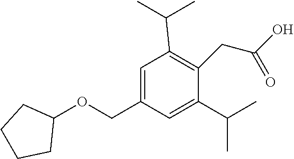 [SMILES: CC(C)c1cc(COC2CCCC2)cc(C(C)C)c1CC(=O)O] | 2-(4-((cyclopentyloxy)methyl)-2,6- diisopropylphenyl)acetic acid | 317 |

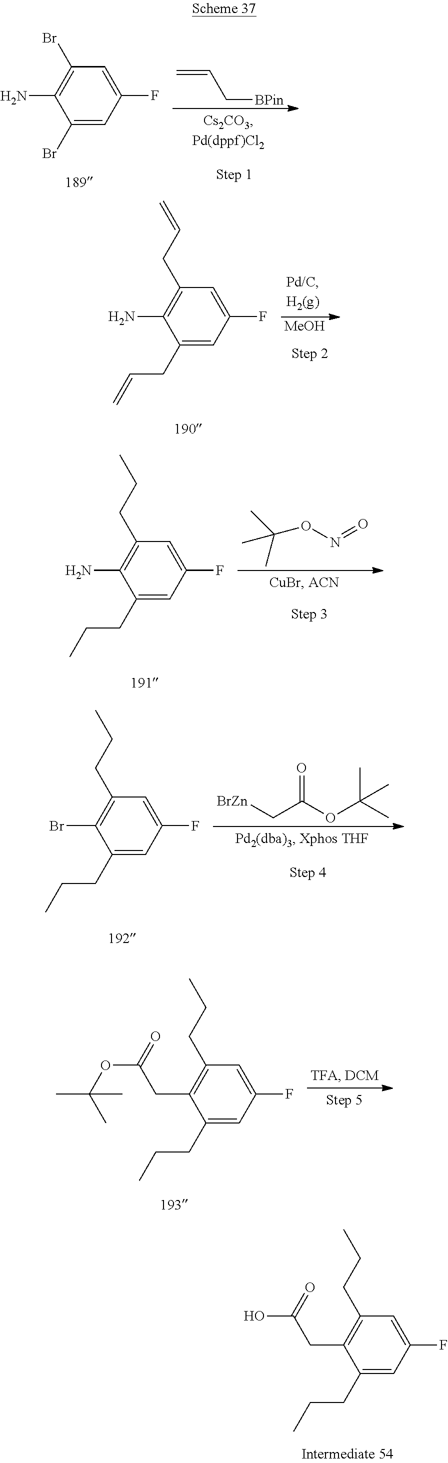

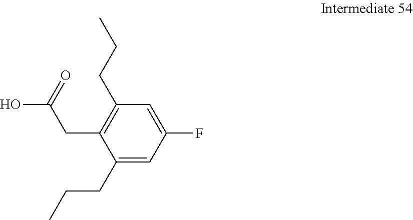 [SMILES: CCCc1cc(F)cc(CCC)c1CC(=O)O]

### 2-(4-Fluoro-2,6-dipropylphenyl)acetic acid

### Step 1: 2,6-Diallyl-4-fluoroaniline

Into a 250-mL round-bottom flask purged and maintained with an inert atmosphere of nitrogen, was placed 2-bromo-4-fluoro-6-(prop-2-en-1-yl)aniline (3.7 g, 16.1 mmol) in THF (100 mL), to the stirred solution was added 4,4, 5, 5-tetramethyl-2-(prop-2-en-1-yl)-1,3,2-dioxaborolane (8.1 g, 48.2 mmol), Cs₂CO₃ (15.7 g, 48.2 mmol) and Pd(dppf)Cl₂ (588 mg, 0.80 mmol). The resulting solution was stirred overnight at 70′C in an oil bath. The resulting mixture was concentrated under vacuum. The residue was eluted from silica gel with ethyl acetate/petroleum ether (1:10). This resulted in 2.6 g (84.5%) of the title compound as yellow oil. MS-ESI: 192 [M+1].

### Step 2: 4-Fluoro-2,6-dipropylaniline

Into a 100-mL round-bottom flask, was placed 4-fluoro-2,6-bis(prop-2-en-1-yl)aniline (2.6 g, 13.59 mmol) in methanol (50 mL). To the stirred solution was added Pd/C (10% wt., 300 mg). The flask was evacuated and filled three times with hydrogen. The resulting solution was stirred 5h at RT under hydrogen. The solids were filtered out. The resulting mixture was concentrated under vacuum. This resulted in 2.5 g (94.1%) of the title compound as light yellow oil. MS-ESI: 196 [M+1]

### Step 3: 2-Bromo-5-fluoro-1,3-dipropylbenzene

Into a 100-mL round-bottom flask purged and maintained with an inert atmosphere of nitrogen, was placed 4-fluoro-2,6-dipropylaniline (840 mg, 4.30 mmol) in ACN (20 mL). To the stirred solution was added CuBr (1.2 g, 8.60 mmol) and tert-butyl nitrite (888 mg, 8.61 mmol). The resulting solution was stirred for 3 h at 60° C. in an oil bath. The resulting mixture was concentrated under vacuum. The residue was eluted from silica gel with petroleum ether. This resulted in 640 mg (57.4%) of the title compound as light yellow oil. MS-ESI: 259/261[M+1].

### Step 4: Tert-butyl 2-(4-fluoro-2,6-dipropylphenyl)acetate

Into a 100-mL round-bottom flask purged and maintained with an inert atmosphere of nitrogen, was placed 2-bromo-5-fluoro-1,3-dipropylbenzene (460 mg, 1.77 mmol) in THE (10 mL). To the mixture was added Xphos (85 mg, 0.18 mmol) and Pd₂(dba)₃ (82 mg, 0.09 mmol). The resulting solution was stirred for 30 min at RT. Then to the above was added tert-butyl 2-(bromozincio)acetate (1.4 g, 5.32 mmol). The resulting solution was stirred for 3 h at 65° C. in an oil bath. The reaction was then quenched by the addition of 10 mL of NH₄Cl (sat.). The resulting solution was extracted with 3×10 mL of DCM. The organic layers were combined, dried over anhydrous sodium sulfate, and concentrated under vacuum. This resulted in 300 mg (57.4%) of the title compound as light yellow oil. MS-ESI: 295 [M+1].

### Step 5: 2-(4-Fluoro-2,6-dipropylphenyl)acetic acid

Into a 50-mL round-bottom flask, was placed tert-butyl 2-(4-fluoro-2,6-dipropylphenyl)acetate (300 mg) in DCM (4 mL) and TFA (2 mL). The resulting solution was stirred for 2 h at RT. The resulting mixture was concentrated under vacuum. The crude product was purified by Prep-TLC with ethyl acetate/petroleum ether (1:3). This resulted in 165 mg (67.9%) of the title compound as a light yellow solid. MS-ESI: 239 [M+1]

TABLE 17
The Intermediates in the following Table were prepared using the similar procedures for converting
compound 189″ to Intermediate 54 shown in Scheme 37 from appropriated starting materials.

| Intermediate | | | Exact Mass |
|---|---|---|---|
| # | Structure | IUPAC Name | [M + H]⁺ |
| Intermediate 55 | 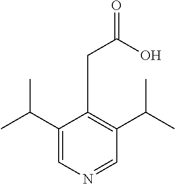 [SMILES: CC(C)c1cncc(C(C)C)c1CC(=O)O] | 2-(3,5-diisopropylpyridin-4- yl)acetic acid | 222 |
| Intermediate 56 | 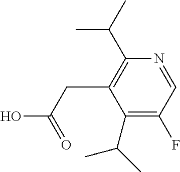 [SMILES: CC(C)c1ncc(F)c(C(C)C)c1CC(=O)O] | 2-(5-fluoro-2,4-diisopropylpyridin- 3-yl)acetic acid | 240 |
| Intermediate 57 | 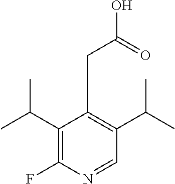 [SMILES: CC(C)c1cnc(F)c(C(C)C)c1CC(=O)O] | 2-(2-fluoro-3,5-diisopropylpyridin- 4-yl)acetic acid | 240 |

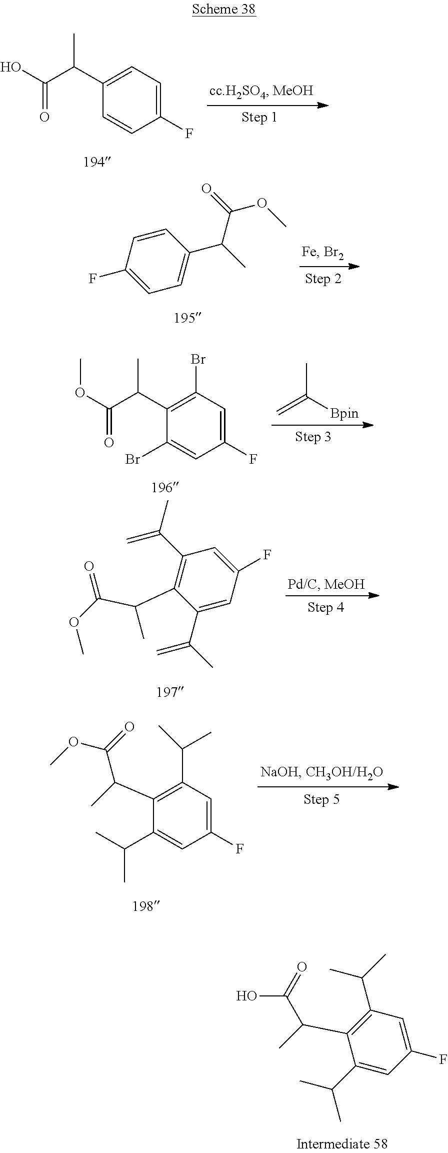

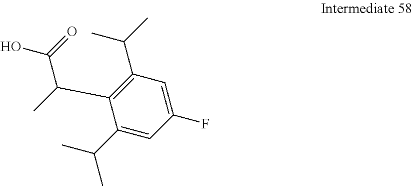 [SMILES: CC(C)c1cc(F)cc(C(C)C)c1C(C)C(=O)O]

### 2-(4-Fluoro-2,6-diisopropylphenvhlpropanoic acid

### Step 1: Methyl 2-(4-fluorophenyl)propanoate

Into a 100-mL round-bottom flask, was placed a solution of 2-(4-fluorophenyl)propanoic acid (2 g, 11.89 mmol) in methanol (20 mL). To the mixture conc. H₂SO₄ (0.05 mL) was added. The resulting solution was stirred for 16 h at 85′C. The reaction was then quenched by the addition of 100 mL of water. The resulting solution was extracted with 3×50 mL of dichloromethane and the organic layers combined. The solids were filtered out. The mixture was dried over anhydrous sodium sulfate and concentrated under vacuum. This resulted in 2.1 g (97%) of the title compound as yellow oil. MS-ESI: 183 [M+1].

### Step 2: Methyl 2-(2,6-dibromo-4-fluorophenyl)propanoate

Into a 100-mL round-bottom flask purged and maintained with an inert atmosphere of nitrogen, was placed a solution of methyl 2-(4-fluorophenyl)propanoate (1.7 g, 9.33 mmol) in CHCl₃ (20 mL). To the stirred solution was added Fe powder (0.21 g) and Br₂ (1.92 mL). The resulting solution was stirred for 16 h at 50° C. The reaction was then quenched by the addition of 50 mL of saturated Na₂S₂O₃ solution. The resulting solution was extracted with 3×50 mL of DCM and the organic layers were combined, dried over anhydrous sodium sulfate and concentrated under vacuum. This resulted in 1.03 g (32%) of the title compound as yellow crude oil. MS-ESI: 339/341 [M+1].

### Step 3: Methyl 2-(4-fluoro-2,6-di(prop-1-en-2-yl)phenyl)propanoate

Into a 40-mL sealed tube purged and maintained with an inert atmosphere of nitrogen, was placed a solution of methyl 2-(2,6-dibromo-4-fluorophenyl)propanoate (1.03 g, 3.03 mmol) in dioxane (10 mL) and H₂O (1 mL). To the stirred solution was added CS₂CO₃ (2 g, 6.14 mmol), Pd(dppf)Cl₂ (230 mg, 0.31 mmol) and 4,4,5,5-tetramethyl-2-(prop-1-en-2-yl)-1,3,2-dioxaborolane (1.07 g, 6.37 mmol). The resulting solution was stirred for 6 h at I 10′C. The resulting mixture was concentrated under vacuum. The residue was eluted from silica gel with ethyl acetate/petroleum ether (1:6). This resulted in 754 mg (95%) of the title compound as yellow oil. MS-ESI: 263 [M+1].

### Step 4: Methyl 2-(4-fluoro-2,6-diisopropylphenyl)propanoate

Into a 50-mL round-bottom flask purged and maintained with an inert atmosphere of H₂, was placed a solution of methyl 2-[4-fluoro-2,6-bis(prop-1-en-2-yl)phenyl]propanoate (820 mg, 3.13 mmol) in methanol (20 mL). To the stirred solution was added Pd/C (10% wt., 0.2 g). The resulting solution was stirred for 4 h at RT. The solids were filtered out. The resulting mixture was concentrated under vacuum. This resulted in 700 mg (84%) of the title compound as yellow crude oil. MS-ESI: 267 [M+1].

### Step 5: 2-(4-Fluoro-2,6-diisopropylphenyl)propanoic acid

Into a 40-mL sealed tube, was placed a solution of methyl 2-[4-fluoro-2,6-bis(propan-2-yl)phenyl]propanoate (300 mg, 1.13 mmol) in 6 M sodium hydroxide (3 mL) and MeOH (3 mL). The resulting solution was stirred for 3 h at 90° C. The reaction was then quenched by the addition of 50 mL of water. The pH value of the solution was adjusted to 2 with hydrogen chloride (1 M). The resulting solution was extracted with 2×50 mL of ethyl acetate and the organic layers combined. The solids were filtered out. The mixture was dried over anhydrous sodium sulfate and concentrated under vacuum. This resulted in 150 mg (53%) of the title compound as yellow oil. MS-ESI: 253 [M+1].

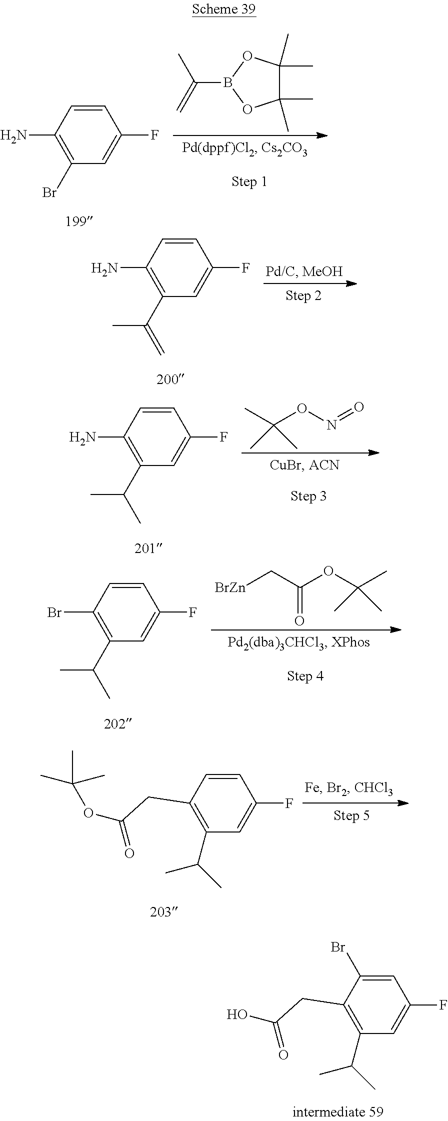

Steps 1-4 used similar procedures for converting compound 189″ to compound 193″ shown in Scheme 37 to afford compound 203″ from compound 199″. MS-ESI: 253 (M+1).

### Step 5: 2-(2-bromo-4-fluoro-6-isopropylphenyl)acetic acid

Into a 100-mL 3-necked round-bottom flask purged and maintained with an inert atmosphere of nitrogen, was placed tert-butyl 2-[4-fluoro-2-(propan-2-yl)phenyl]acetate (1.0 g, 3.96 mmol) in CHCl₃ (25 mL). To the solution was added AcOH (0.01 mL), Fe powder (22.1 mg, 0.40 mmol) and Br₂ (3.17 g, 19.8 mmol). The resulting solution was stirred for 16 h at 50′C in an oil bath. The reaction was then quenched by the addition of 20 mL of Na₂S₂O₃. The resulting solution was extracted with 2×20 ml of ethyl acetate concentrated. The residue was eluted from silica gel with ethyl acetate/petroleum ether (1:2). This resulted in 700 mg (64.2%) of the title compound as a white solid. MS-ESI: 275 [M+1].

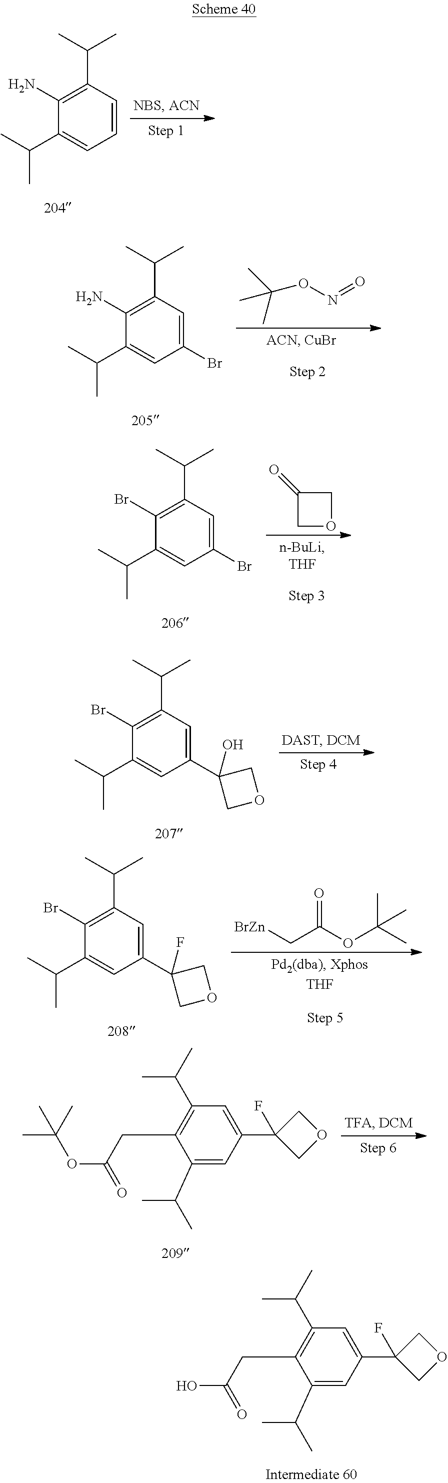

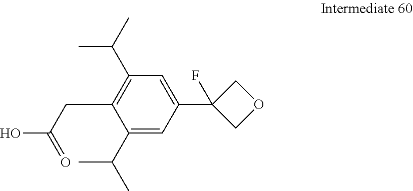 [SMILES: CC(C)c1cc(C2(F)COC2)cc(C(C)C)c1CC(=O)O]

### 2-(4-(3-Fluorooxetan-3-yl)-2,6-diisopropylphenyl)acetic acid

### Step 1: 4-Bromo-2,6-diisopropylaniline

Into a 500-mL round-bottom flask, was placed 2,6-bis(propan-2-yl)aniline (10 g, 56.4 mmol) in ACN (200 mL), to the stirred solution was added NBS (11.0 g, 62.0 mmol). The resulting solution was stirred overnight at RT. The resulting mixture was concentrated under vacuum. The residue was eluted from silica gel with petroleum ether. This resulted in 9.5 g (65.7%) of the title compound as brown oil. MS-ESI: 256/258 [M+1].

### Step 2: 2, 5-Dibromo-1,3-diisopropylbenzene

Into a 500-mL round-bottom flask purged and maintained with an inert atmosphere of nitrogen, was placed 4-bromo-2,6-bis(propan-2-yl)aniline (6.4 g, 24.9 mmol) in ACN (200 mL). To the stirred solution was added CuBr (7.2 g, 50.2 mmol) and tert-butyl nitrite (5.2 g, 50.5 mmol). The resulting solution was stirred for 3 h at 65° C. in an oil bath. The resulting mixture was concentrated under vacuum. The residue was eluted from silica gel with petroleum ether. This resulted in 5 g (62.5%) of the title compound as light yellow oil. MS-ESI: 319/321/323 [M+1].

### Step 3: 3-(4-Bromo-3, 5-diisopropylphenyl)oxetan-3-ol

Into a 100-mL 3-necked round-bottom flask purged and maintained with an inert atmosphere of nitrogen, was placed 2, 5-dibromo-1,3-bis(propan-2-yl)benzene (5 g, 15.6 mmol) in THF (50 mL). This was followed by the addition of n-BuLi (2.5 M, 6.25 mL, 15.6 mmol) dropwise with stirring at −78° C. The resulting solution was stirred for 30 min at −78° C. To the above was added a solution of oxetan-3-one (1.13 g, 15.6 mmol) in THF (2 mL) dropwise with stirring at −78° C. The resulting solution was slowly warmed to RT and stirred for 2 h at RT. The reaction was then quenched by the addition of 100 mL of NH₄Cl (sat.). The resulting solution was extracted with 3×100 mL of ethyl acetate and the organic layers combined and dried over anhydrous sodium sulfate and concentrated under vacuum. The residue was eluted from silica gel with ethyl acetate/petroleum ether (1:3). The crude product was purified by Flash-Prep-HPLC with the following conditions: Column, Cis silica gel; mobile phase, H₂O (0.1% FA) and ACN (40% to 70% ACN gradient in 30 min), Detector, UV 254/210 nm. This resulted in 1.25 g (25.5%) of the title compound as a white solid. MS-ESI: 313/315[M+1].

### Step 4: 3-(4-Bromo-3, 5-diisopropylphenyl)-3-fluorooxetane

Into a 50-mL 3-necked round-bottom flask purged and maintained with an inert atmosphere of nitrogen, was placed 3-[4-bromo-3, 5-bis(propan-2-yl)phenyl]oxetan-3-ol (600 mg, 1.92 mmol) in DCM (10 mL). This was followed by the addition of DAST (618 mg, 3.83 mmol) dropwise with stirring at 0° C. The resulting solution was stirred overnight at RT. The reaction was then quenched by the addition of 5 mL of methanol. The resulting mixture was concentrated under vacuum. The residue was eluted from silica gel with ethyl acetate/petroleum ether (1:10). This resulted in 430 mg (71.2%) of the title compound as a white solid. MS-ESI: 315/317[M+1].

### Step 5: Tert-butyl 2-(4-(3-fluorooxetan-3-yl)-2,6-diisopropylphenyl)acetate

Into a 100-mL round-bottom flask, was placed 3-[4-bromo-3, 5-bis(propan-2-yl)phenyl]-3-fluorooxetane (420 mg, 1.33 mmol) in THF (20 mL), to the mixture was added Xphos (60 mg, 0.13 mmol) and Pd₂(dba); (61 mg, 0.07 mmol). The resulting solution was stirred for 30 min at RT. Then to the above was added tert-butyl 2-(bromozincio)acetate (694.0 mg, 2.66 mmol). The resulting solution was stirred for 3 h at 60° C. in an oil bath. The resulting mixture was concentrated under vacuum. The residue was eluted from silica gel with ethyl acetate/petroleum ether (1:10). resulted in 450 mg (96.3%) of the title compound as a light yellow solid. MS-ESI: 351 [M+1].

### Step 6: 2-(4-(3-Fluorooxetan-3-yl)-2,6-diisopropylphenyl)acetic acid

Into a 50-mL round-bottom flask, was placed tert-butyl 2-[4-(3-fluorooxetan-3-yl)-2,6-bis(propan-2-yl) phenyl]acetate (450 mg, 1.28 mmol) in DCM (4 mL) and TFA (2 mL). The resulting solution was stirred overnight at RT. The resulting mixture was concentrated under vacuum. The residue was eluted from silica gel with ethyl acetate/petroleum ether (1:3). This resulted in 300 mg (79.3%) of the title compound as a light yellow solid. MS-ESI: 295[M+1].

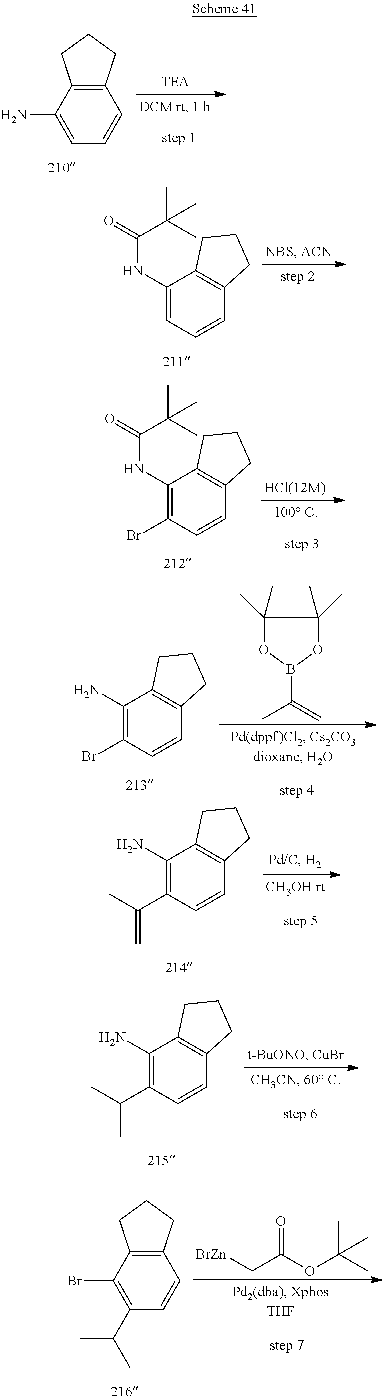

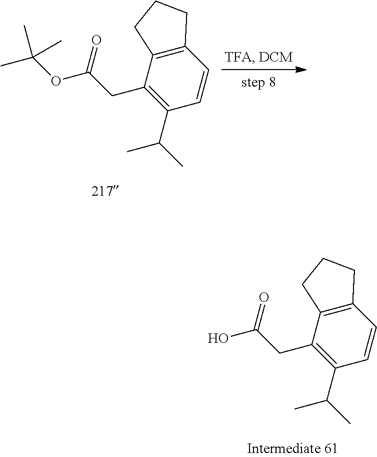 [SMILES: CC(C)c1ccc2c(c1CC(=O)OC(C)(C)C)CCC2 ; CC(C)c1ccc2c(c1CC(=O)O)CCC2]

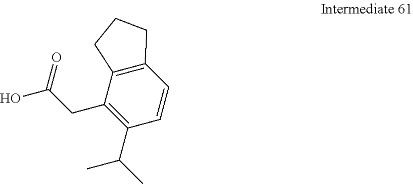 [SMILES: CC(C)c1ccc2c(c1CC(=O)O)CCC2]

### 2-(5-Isopropyl-2,3-dihydro-1H-inden-4-yl)acetic acid

### Step 1: N-(2,3-dihydro-1H-inden-4-yl)pivalamide

Into a 500-mL round-bottom flask, was placed 2,3-dihydro-1H-inden-4-amine (10 g, 75.1 mmol) in DCM (100 mL), to the stirred solution was added 2,2-dimethylpropanoyl chloride (9.05 g, 75.1 mmol) and TEA (11.4 g, 112 mmol). The resulting solution was stirred for 1 h at RT. The reaction was then quenched by the addition of 100 mL of water. The resulting solution was extracted with 3×100 ml of DCM and dried over anhydrous sodium sulfate and concentrated. This resulted in 15 g (91.9%) of the title compound as an off-white solid. MS-ESI: 218 [M+1].

### Step 2: N-(5-bromo-2,3-dihydro-1H-inden-4-yl)pivalamide

Into a 500-mL round-bottom flask purged and maintained with an inert atmosphere of nitrogen, was placed a solution of N-(2,3-dihydro-1H-inden-4-yl)-2,2-dimethylpropanamide (9 g, 41.5 mmol) in ACN (200 mL). To above solution was added NBS (8.86 g, 49.8 mmol). The resulting solution was stirred for 15 h at RT, after which it was extracted with 3×200 ml of DCM. The organic layers were combined, washed with 3×200 ml of aq. Na₂CO₃, dried over anhydrous sodium sulfate, and concentrated. This resulted in 12 g of the title compound as a brown solid. MS-ESI: 296/298 [M+1]

### Step 3: 5-Bromo-2,3-dihydro-1H-inden-4-amine

Into a 500-mL round-bottom flask, was placed a solution of N-(5-bromo-2,3-dihydro-1H-inden-4-yl)-2,2-dimethylpropanamide (10 g, 33.8 mmol) in HCl (200 mL). The resulting solution was stirred for 15 h at 100° C. The reaction was then quenched by the addition of water. The resulting solution was extracted with 3×500 mL of ethyl acetate dried over anhydrous sodium sulfate and concentrated under vacuum. The residue was eluted from silica gel with ethyl acetate/petroleum ether (1:10). This resulted in 7 g (97.7%) of the title compound as a brown solid. MS-ESI: 212/214[M+1].

### Step 4: 5-(Prop-1-en-2-yl)-2,3-dihydro-1H-inden-4-amine

Into a 500-mL round-bottom flask purged and maintained with an inert atmosphere of nitrogen, was placed a solution of 5-bromo-2,3-dihydro-1H-inden-4-amine (7 g, 33 mmol) in dioxane (250 mL) and H₂O (25 mL). To the above was added 4,4, 5, 5-tetramethyl-2-(prop-1-en-2-yl)-1,3,2-dioxaborolane (8.32 g, 49.5 mmol), Cs₂CO₃ (32.2 g, 99.0 mmol) and Pd(dppf)Cl₂ (2.41 g, 3.3 mmol). The resulting solution was stirred for 15 h at 95° C. The reaction was then quenched by the addition of water. The resulting solution was extracted with 3×200 mL of DCM and concentrated. The residue was eluted from silica gel with ethyl acetate/petroleum ether (1:10). This resulted in 4 g (69.9%) of the title compound as a brown solid. MS-ESI: 174 [M+1].

### Step 5: 5-isopropyl-2,3-dihydro-1H-inden-4-amine

Into a 250-mL round-bottom flask, was placed a solution of 5-(prop-1-en-2-yl)-2,3-dihydro-1H-inden-4-amine (4 g, 23.09 mmol) in methanol (100 mL), to the stirred solution was added Pd/C (10% wt., 400 mg). The flask was evacuated and flushed three times with hydrogen. The resulting solution was stirred overnight at RT under hydrogen. The solids were filtered out. The resulting mixture was concentrated under vacuum. This resulted in 4 g (98.8%) of the title compound as a brown solid. MS-ESI: 176 [M+1].

Steps 6-8 used similar procedures for converting compound 191″ to intermediate 54 shown in Scheme 37 to afford intermediate 61 from compound 215″. MS-ESI: 219 (M+1).

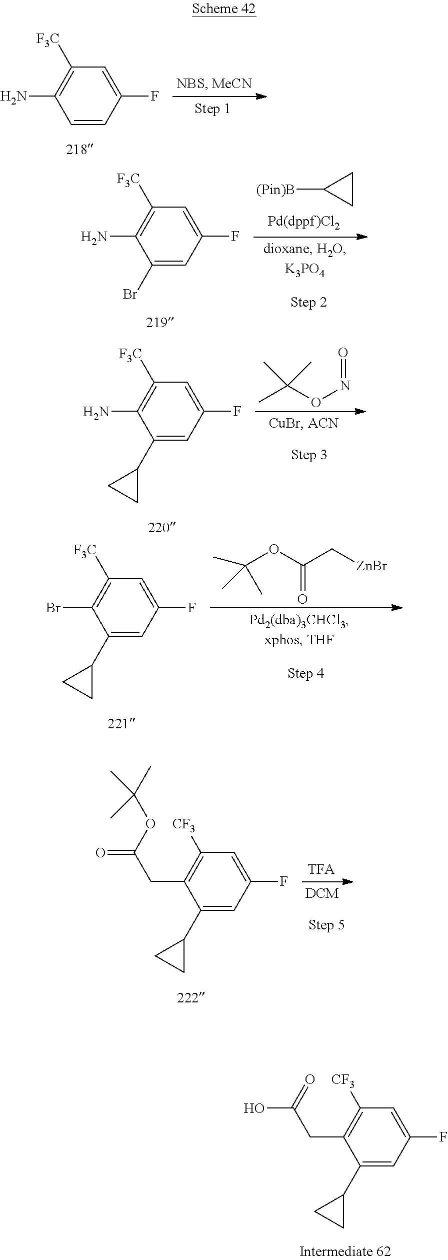

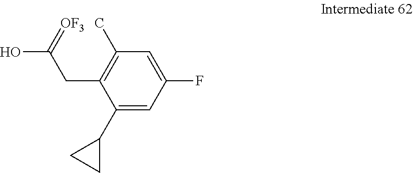

### 2-(2-Cyclopropyl-4-fluoro-6-(trifluoromethyl)phenyl)acetic acid

### Step 1: 2-Bromo-4-fluoro-6-(trifluoromethyl)aniline

Into a 250-mL round-bottom flask, was placed 4-fluoro-2-(trifluoromethyl)aniline (11.6 g, 64.7 mmol) in ACN (100 mL). This was followed by the addition of NBS (12.6 mg, 71.2 mmol) in portions with stirring at 0° C. The resulting solution was stirred for 2 h at RT. The reaction was then quenched by the addition of water. The resulting solution was extracted with 3×100 mL of ethyl acetate. The residue was eluted from silica gel with ethyl acetate/petroleum ether (1:20). This resulted in 13 g (77.8%) of the title compound as a red solid. MS-ESI: 258/260 (M+1).

### Step 2: 2-Cyclopropyl-4-fluoro-6-(trifluoromethyl)aniline

Into a 500-mL round-bottom flask purged and maintained with an inert atmosphere of nitrogen, was placed 2-bromo-4-fluoro-6-(trifluoromethyl)aniline (10 g, 38.7 mmol) in dioxane (200 mL) and H₂O (10 mL). To the stirred solution was added K₃PO₄ (24.6 g, 116.2 mmol), Pd(dppf)Cl₂ (2.84 g, 3.88 mmol) and cyclopropylboronic acid or ester (4.99 g, 58.1 mmol). The resulting solution was stirred for overnight at 90° C. in an oil bath. The solids were filtered out. The resulting mixture was concentrated. The residue was eluted from silica gel with ethyl acetate/petroleum ether (1:20). This resulted in 7.5 g (88.2%) of the title compound as a yellow oil. MS-ESI: 220 (M+1).

### Step 3: 2-Bromo-1-cyclopropyl-5-fluoro-3-(trifluoromethyl)benzene

Into a 100-mL round-bottom flask, was placed 2-cyclopropyl-4-fluoro-6-(trifluoromethyl)aniline (1.5 g, 6.85 mmol) in ACN (30 mL). To the above solution was added tert-butyl nitrite (1.41 g, 13.7 mmol) and CuBr (1.96 g, 13.7 mmol). The resulting solution was stirred for 3 h at 60′C. The resulting mixture was concentrated. The residue was eluted from silica gel with ethyl acetate/petroleum ether (1:20). This resulted in 1 g (51.6%) of the title compound as a yellow liquid. MS-ESI: 283/285 (M+I).

### Step 4: Tert-butyl 2-[2-cyclopropyl-4-fluoro-6-(trifluoromethyl)phenyl]acetate

Into a 100-mL round-bottom flask purged and maintained with an inert atmosphere of nitrogen, was placed 2-bromo-1-cyclopropyl-5-fluoro-3-(trifluoromethyl)benzene (360 mg, 1.27 mmol) in THF (10 mL), Xphos (121.26 mg, 0.25 mmol), Pd₂(dba)₃·CHCl₃ (131.6 mg, 0.13 mmol), and tert-butyl 2-(bromozincio)acetate (662.4 mg, 2.54 mmol). The resulting solution was stirred for 2 h at 65° C. The resulting mixture was concentrated. The residue was eluted from silica gel with ethyl acetate/petroleum ether (1:20). This resulted in 300 mg (74.1%) of the title compound as yellow oil. MS-ESI: 319 (M+1).

### Step 5: 2-[2-Cyclopropyl-4-fluoro-6-(trifluoromethyl)phenyl]acetic acid

Into a 50-mL round-bottom flask, was placed tert-butyl 2-[2-cyclopropyl-4-fluoro-6-(trifluoromethyl) phenyl]acetate (300 mg, 0.94 mmol) in TFA (2 mL) and DCM (2 mL). The resulting solution was stirred for 2 h at RT. The resulting mixture was concentrated. The residue was eluted from silica gel with ethyl acetate/petroleum ether (1:5). This resulted in 230 mg (93.0%) of the title compound as a yellow solid. MS-ESI: 263 (M+1).

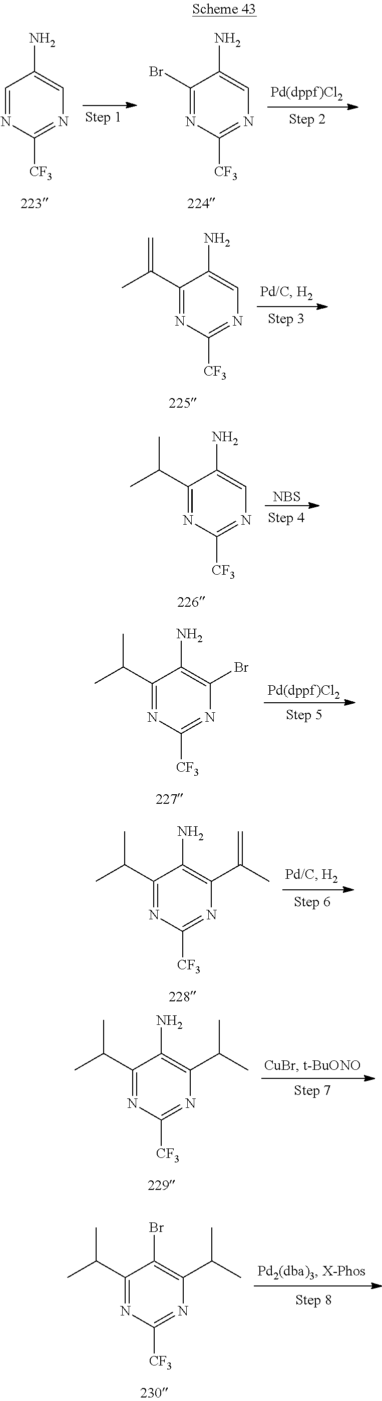

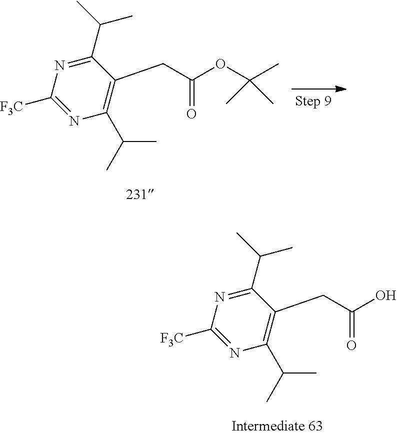 [SMILES: CC(C)c1nc(C(F)(F)F)nc(C(C)C)c1CC(=O)OC(C)(C)C ; CC(C)c1nc(C(F)(F)F)nc(C(C)C)c1CC(=O)O]

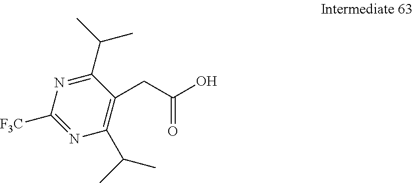 [SMILES: CC(C)c1nc(C(F)(F)F)nc(C(C)C)c1CC(=O)O]

### 2-(4,6-Diisopropyl-2-(trifluoromethyl)pyrimidin-5-yl)acetic acid

### Step 1: 4-Bromo-2-(trifluoromethyl)pyrimidin-5-amine

Into a 100-mL round-bottom flask purged and maintained with an inert atmosphere of nitrogen, was placed 2-(trifluoromethyl)pyrimidin-5-amine (2 g, 12.3 mmol) in acetonitrile (20 mL), to this stirred solution was added NBS (2.62 g, 14.7 mmol). The resulting solution was stirred for 12 h at RT. The resulting solution was diluted with 40 mL of water. The resulting solution was extracted with 2×30 mL of dichloromethane and concentrated. The residue was eluted from silica gel with ethyl acetate/petroleum ether (1:50 to 1:20). This resulted in 1.6 g (53.9/6) of the title compound as a brown solid. MS-ESI: 242/244 [M+1]

### Step 2: 4-(Prop-1-en-2-yl)-2-(trifluoromethyl)pyrimidin-5-amine

Into a 100-mL round-bottom flask purged and maintained with an inert atmosphere of nitrogen, was placed 4-bromo-2-(trifluoromethyl)pyrimidin-5-amine (1.6 g, 6.61 mmol) in dioxane (20 mL). This was followed by the addition of 4,4,5,5-tetramethyl-2-(prop-1-en-2-yl)-1,3,2-dioxaborolane (1.44 g, 8.57 mmol), Pd(dppf)Cl₂ (241 mg, 0.33 mmol), and Cs₂CO₃ (3.23 g, 9.92 mmol). The resulting solution was stirred for 14 h at 100° C. in an oil bath. The resulting solution was diluted with 40 mL of water. The resulting solution was extracted with 3×30 mL of DCM and concentrated. The residue was eluted from silica gel with ethyl acetate/petroleum ether (1:5). This resulted in 1.1 g (81.8%) of the title compound as a brown solid. MS-ESI: 204 [M+1].

### Step 3: 4-isopropyl-2-(trifluoromethyl)pyrimidin-5-amine

Into a 100-mL round-bottom flask purged and maintained with an inert atmosphere of nitrogen, was placed 4-(prop-1-en-2-yl)-2-(trifluoromethyl)pyrimidin-5-amine (1.2 g, 5.91 mmol) in methanol (20 mL), to the stirred solution was added Pd/C (10% wt., 200 mg). The flask was evacuated and filled three times with hydrogen. The resulting solution was stirred 16h at RT under hydrogen. The solids were filtered out. The resulting mixture was concentrated under vacuum. This resulted in 1.1 g (90.8%) of the title compound as a brown solid. MS-ESI: 206 [M+1].

### Step 4: 4-Bromo-6-isopropyl-2-(trifluoromethyl)pyrimidin-5-amine

Into a 50-mL round-bottom flask purged and maintained with an inert atmosphere of nitrogen, was placed 4-(propan-2-yl)-2-(trifluoromethyl)pyrimidin-5-amine (1.1 g, 5.36 mmol) in acetonitrile (20 mL), to this solution was added NBS (1.15 g, 6.46 mmol) in portions with stirring. The resulting solution was stirred for 12 h at RT. The resulting solution was diluted with 40 mL of water. The resulting solution was extracted with 2×30 mL of DCM concentrated. The residue was eluted from silica gel with ethyl acetate/petroleum ether (1:40 to 1:30). This resulted in 1.0 g (65.6%) of the title compound as a brown solid. MS-ESI: 284/286 [M+1].

Steps 5-9 used similar procedures for converting compound 189″ to intermediate 54 shown in Scheme 37 to afford intermediate 63 from compound 227″. MS-ESI: 291 (M+1).

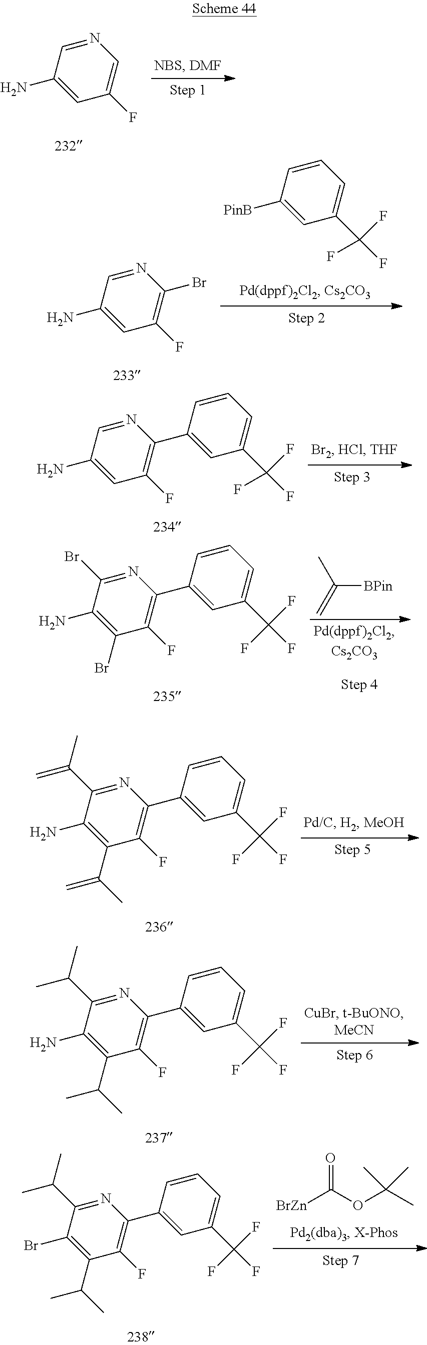

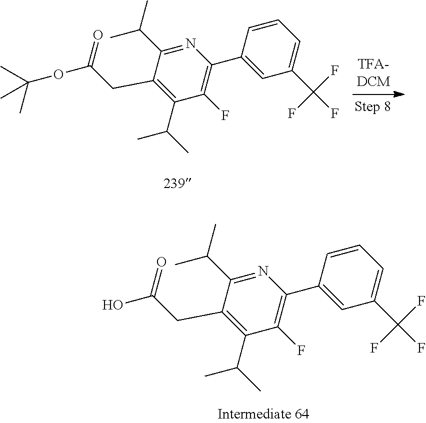 [SMILES: CC(C)c1nc(-c2cccc(C(F)(F)F)c2)c(F)c(C(C)C)c1CC(=O)OC(C)(C)C ; CC(C)c1nc(-c2cccc(C(F)(F)F)c2)c(F)c(C(C)C)c1CC(=O)O]

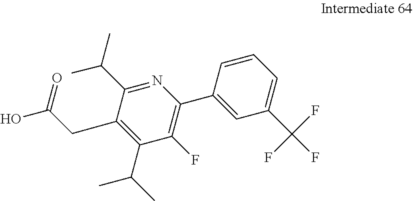 [SMILES: CC(C)c1nc(-c2cccc(C(F)(F)F)c2)c(F)c(C(C)C)c1CC(=O)O]

### 2-(5-Fluoro-2,4-diisopropyl-6-(3-(trifluoromethyl)phenyl)pyridin-3-yl)acetic acid

### Step 1: 6-Bromo-5-fluoropyridin-3-amine

Into a 100 mL round-bottom flask, was added 5-fluoropyridin-3-amine (2 g, 17.9 mmol) in DMF (15 mL) at RT. To the stirred solution was added NBS (3.19 g, 17.9 mmol) in DMF (5 mL) dropwise at RT. The resulting solution was stirred for 1h at RT and diluted with water (75 mL). The resulting mixture was extracted with ethyl acetate (3×20 mL). The combined organic layers were dried over anhydrous Na₂SO₄. After filtration, the filtrate was concentrated under reduced pressure. The residue was purified by silica gel chromatography, eluted with EtOAc/petroleum ether (8:1) to afford the title compound (3 g, 79%) as a dark yellow solid. ME-ESI: 191/193 [M+1].

### Step 2: s5-Fluoro-6-(3-(trifluoromethyl)phenyl)pyridin-3-amine

Into a 500-mL round-bottom flask purged and maintained with an inert atmosphere of nitrogen, was added 6-bromo-5-fluoropyridin-3-amine (3 g, 15.7 mmol) in dioxane (200 mL) and H₂O (20 mL) at RT. To the stirred solution were added Pd(dppf)Cl₂ (1.15 g, 1.57 mmol) and Cs₂CO₃ (10.2 g, 31.4 mmol) at RT under nitrogen atmosphere. Then 4,4,5,5-tetramethyl-2-[3-(trifluoromethyl)phenyl]-1,3,2-dioxaborolane (17.1 g, 62.8 mmol) was added to the above mixture. After the addition was complete and the resulting mixture was stirred at 80° C. in an oil bath overnight. The mixture was concentrated and applied into silica gel and eluted with ethyl acetate/petroleum ether (12:1) to afford the title compound (4.1 g, 94.7%) as a yellow oil. MS-ESI: 257 [M+1].

### Step 3: 2,4-Dibromo-5-fluoro-6-(3-(trifluoromethyl)phenyl)pyridin-3-amine

Into a 250 mL round-bottom flask, were added 5-fluoro-6-[3-(trifluoromethyl)phenyl]pyridin-3-amine (4.1 g, 16.0 mmol) in THF (25 mL) at RT. To the stirred solution was added HCl (2 M, 13.5 mL) in one portion at RT. To this mixture was added Br₂ (2.4 mL) dropwise. After the addition was complete, the resulting mixture was stirred for 4 h at RT. The resulting mixture was extracted with ethyl acetate (3×90 mL). The combined organic layers were dried over anhydrous Na₂SO₄. After filtration, the filtrate was concentrated under reduced pressure. The residue was purified by silica gel chromatography, eluted with EtOAc/petroleum ether (20:1) to afford the title compound (2.25 g, 33.9%) as a yellow solid. ME-ESI: 413/415/417 [M+1].

Steps 4-8 used procedures for converting compound 189″ to intermediate 54 shown in Scheme 37 to afford intermediate 64 from compound 235″. MS-ESI: 384 (M+1).

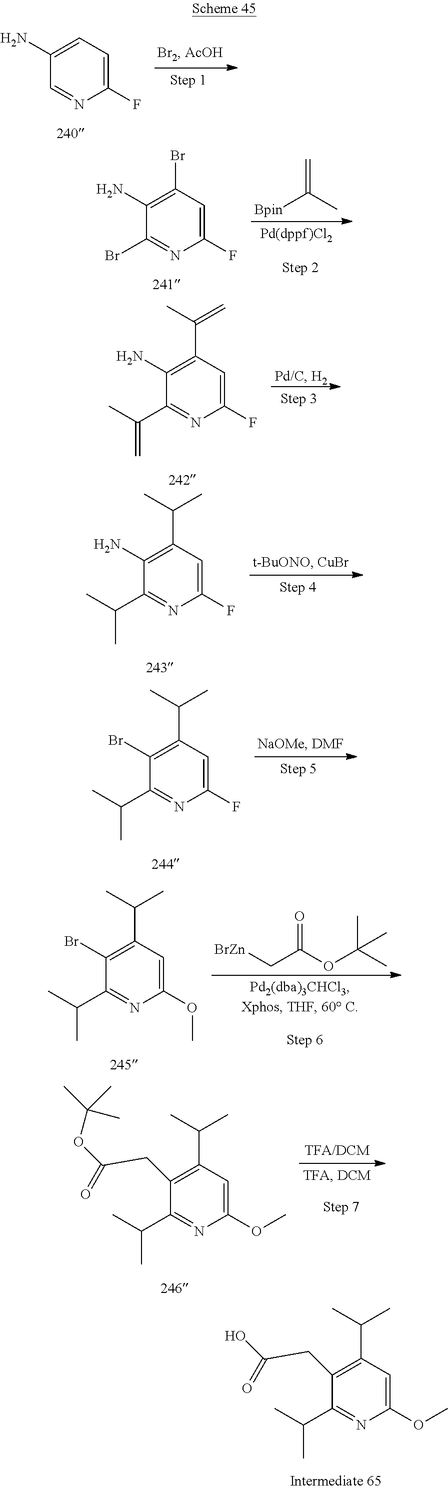

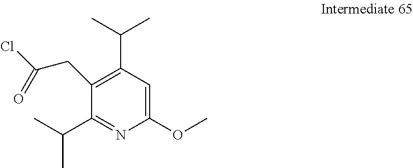 [SMILES: COc1cc(C(C)C)c(CC(=O)Cl)c(C(C)C)n1]

### 2-(2,4-Diisopropyl-6-methoxypyridin-3-yl)acetyl chloride

### Step 1: 2,4-Dibromo-6-fluoropyridin-3-amine

Into a 1-L round-bottom flask, was placed a solution of 6-fluoropyridin-3-amine (4.05 g, 36.1 mmol) in AcOH (40 mL). This was followed by the addition of a solution of Br₂ (4.1 mL, 79.9 mmol) in AcOH (50 mL) dropwise with stirring at 0° C. The resulting solution was stirred for 16 h at RT. The resulting mixture was washed with 150 ml of saturated solution of NaHCO₃, extracted with 3×200 ml of dichloromethane, and dried over anhydrous sodium sulfate. This resulted in 5 g (51.2%) of the title compound as a yellow solid. MS-ESI: 269/271/273[M+1].

Steps 2-4 used similar procedures for converting compound 189 to compound 192 shown in Scheme 37 to afford compound 244 from compound 241. MS-ESI: 260/262 (M+1).

### Step 5: 3-Bromo-2,4-diisopropyl-6-methoxypyridine

Into a 25-mL round-bottom flask, was placed a solution of 3-bromo-6-fluoro-2,4-bis(propan-2-yl)pyridine (130 mg, 0.50 mmol) in DMF (5 mL). To the solution was added CH₃ONa (108 mg, 2.0 mmol). The resulting solution was stirred overnight at RT. The resulting mixture was washed with 20 ml of H₂O. The resulting solution was extracted with 2×25 ml of ethyl acetate dried over anhydrous sodium sulfate and concentrated. The residue was eluted from silica gel with petroleum ether. This resulted in 100 mg (73.5%) of the title compound as yellow oil. MS-ESI: 272/274 [M+1].

Steps 6-7 used similar procedures for converting compound 192″ to intermediate 54 shown in Scheme 37 to afford intermediate 65 from compound 245″. MS-ESI: 252 (M+1).

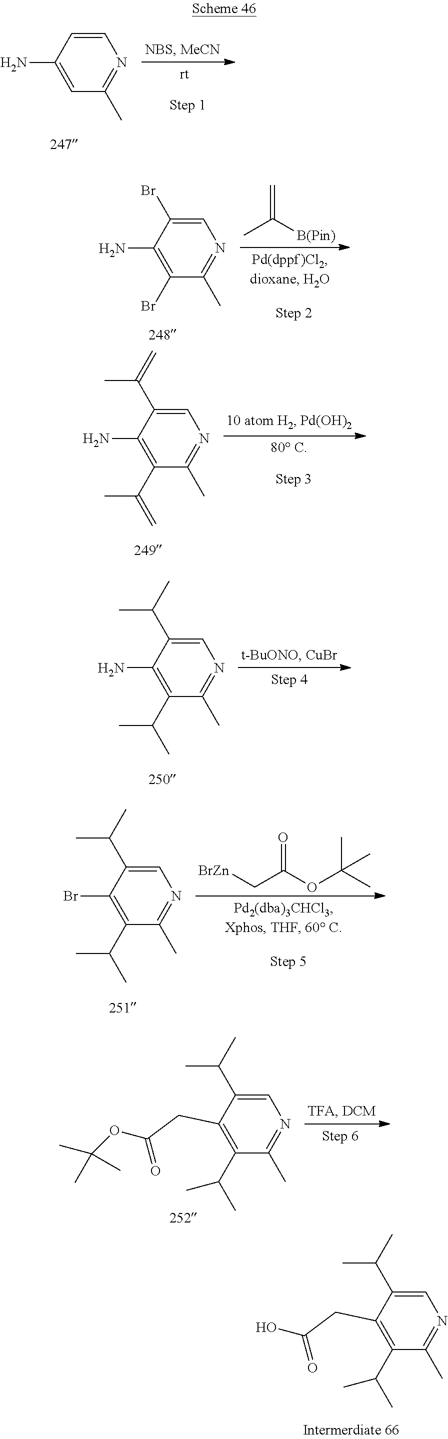

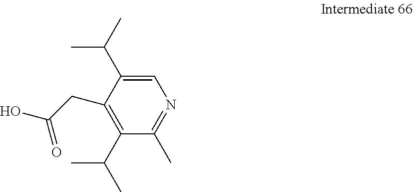 [SMILES: Cc1ncc(C(C)C)c(CC(=O)O)c1C(C)C]

### 2-(3, 5-Diisopropyl-2-methylpyridin-4-yl)acetic acid

Steps 1-2 used similar procedures for converting compound 161″ to compound 163″ shown in Scheme 31 to afford compound 249″ from compound 247″. MS-ESI: 189 (M+1).

### Step 3: 3, 5-Diisopropyl-2-methylpyridin-4-amine

Into a 1-L pressure tank reactor purged and maintained with an inert atmosphere of nitrogen, was placed 2-methyl-3, 5-bis(prop-1-en-2-yl)pyridin-4-amine (22.0 g, 117 mmol) in MeOH (400 mL). To the stirred solution was added Pd(OH)₂/C (10% wt., 2.0 g). The reaction solution was evacuated and filled three times with hydrogen. The resulting mixture was stirred for 3 days at 80° C. under hydrogen atmosphere. The solids were filtered out. The resulting mixture was concentrated under vacuum. This resulted in 17.5 g (77.7%) of the title compound as brown oil. MS-ESI: 193 [M+1].

Steps 4-6 used similar procedures for converting compound 164″ to Intermediate 36 shown in Scheme 31 to afford intermediate 66 from compound 250″. MS-ESI: 235 (M+1).

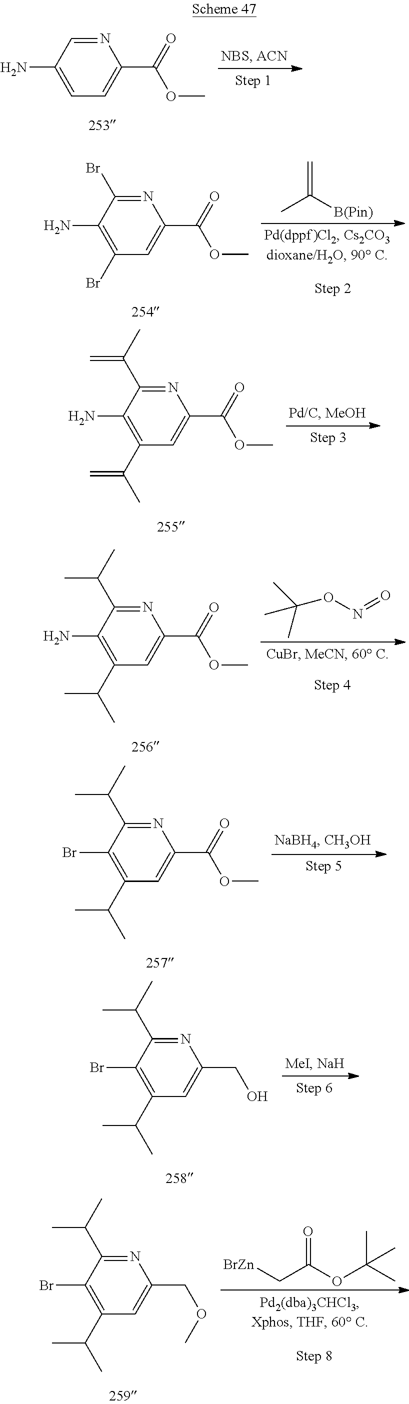

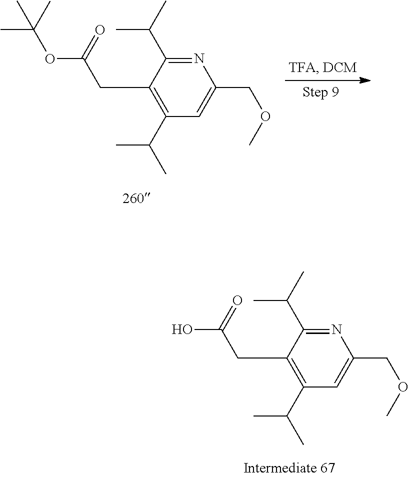 [SMILES: COCc1cc(C(C)C)c(CC(=O)OC(C)(C)C)c(C(C)C)n1 ; COCc1cc(C(C)C)c(CC(=O)O)c(C(C)C)n1]

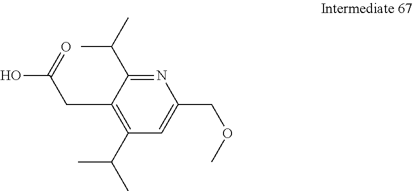 [SMILES: COCc1cc(C(C)C)c(CC(=O)O)c(C(C)C)n1]

### 2-(2,4-Diisopropyl-6-(methoxymethyl)pyridin-3-yl)acetic acid

Steps 1-4 used similar procedures for converting compound 161″ to compound 165″ shown in Scheme 31 to afford compound 257″ from compound 253″. MS-ESI: 300/302 (M+1).

### Step 5: (5-Bromo-4,6-diisopropylpyridine-2-yl)methanol

Into a 50-mL round-bottom flask, was placed methyl 5-bromo-4,6-bis(propan-2-yl)pyridine-2-carboxylate (1.4 g, 4.66 mmol) in methanol (10 mL). This was followed by the addition of NaBH₄ (532 mg, 13.9 mmol) in several batches at 0° C. The resulting solution was stirred for 1 h at RT. The reaction was then quenched by the addition of 20 mL of water. The resulting solution was extracted with 3×30 mL of ethyl acetate concentrated. This resulted in 1.2 g (94.5%) of the title compound as an off-white solid. MS-ESI: 272/274 [M+1].

### Step 6: 3-Bromo-2,4-diisopropyl-6-(methoxymethyl)pyridine

Into a 100-mL round-bottom flask purged and maintained with an inert atmosphere of nitrogen, was placed [5-bromo-4,6-bis(propan-2-yl)pyridin-2-yl]methanol (800 mg, 2.94 mmol) in THF (10 mL). This was followed by the addition of NaH (60/6 wt. oil dispersion, 353 mg, 8.82 mmol) in several batches at 0° C. The resulting solution was stirred for 20 min at RT. To this was added CH₃I (1.25 g, 8.82 mmol) dropwise with stirring at 0° C. The resulting solution was allowed to react, with stirring, overnight at RT. The reaction was then quenched by the addition of 20 mL of water. The resulting solution was extracted with 3×30 mL of dichloromethane and concentrated. The residue was eluted from silica gel with ethyl acetate/petroleum ether (1:10). This resulted in 820 mg (97.4%) of the title compound as light yellow oil.

Steps 8-9 used similar procedures for converting compound 165″ to intermediate 36 shown in Scheme 31 to afford intermediate 67 from compound 259″. MS-ESI: 286/287 (M+1).

### Example 1 (131)

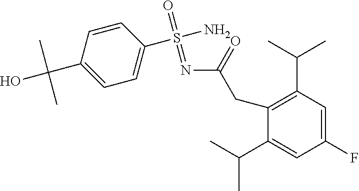 [SMILES: CC(C)c1cc(F)cc(C(C)C)c1CC(=O)N=S(N)(=O)c1ccc(C(C)(C)O)cc1]

### 2-(4-Fluoro-2,6-diisopropylphenyl-N-(4-(2-hydroxypropan-2-yl)phenylsulfonimidoyl)acetamide (Scheme 1)

### Examples 2 (131b) and 3 (131a)

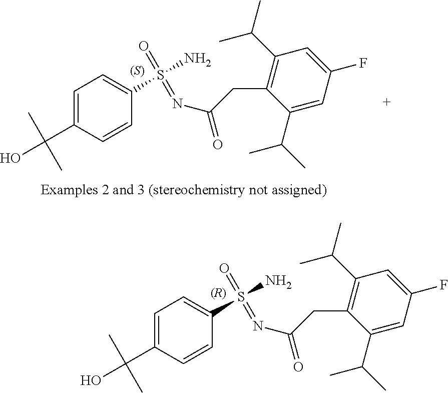

### (S)- and (R)-2-(4-Fluoro-2,6-diisopropylphenyl)-N-(4-(2-hydroxypropan-2-yl)phenylsulfonimidoyl)acetamide

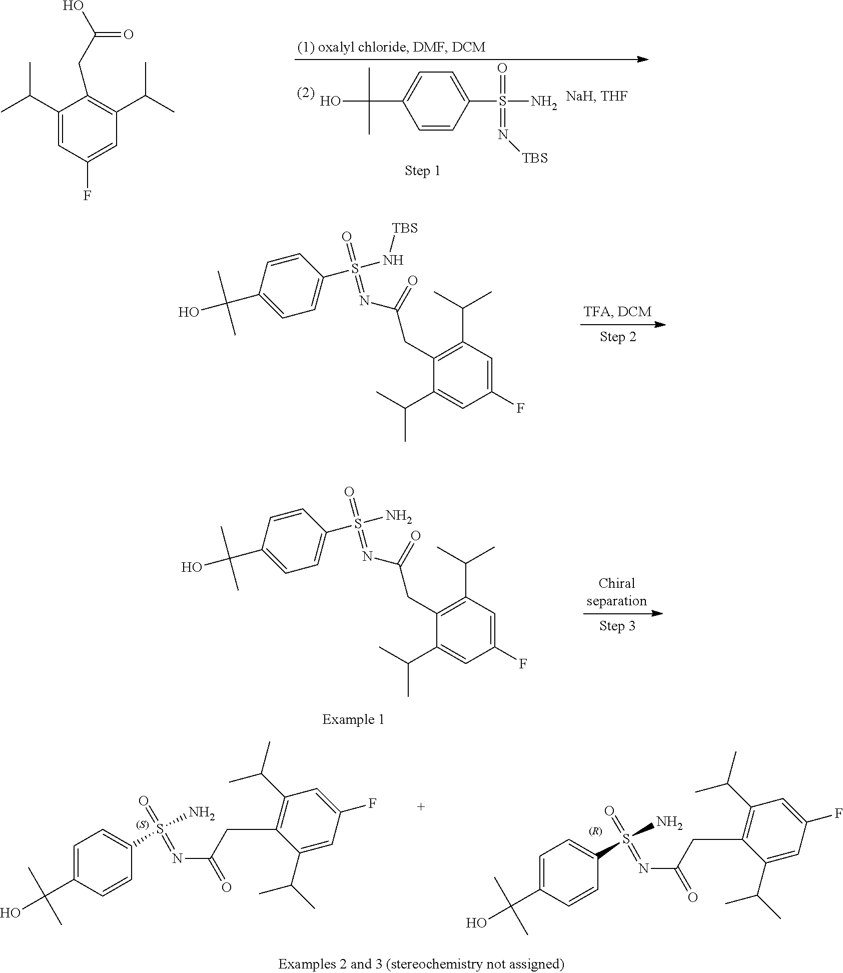

### Step 1: N-(tert-butyldimethylsilylamino-4-(2-hydroxypropan-2-yl)phenylhydrosulfonimidoyl)-2-(4-fluoro-2,6-diisopropylphenyl)acetamide

Into a 50-mL round-bottom flask was placed 2-(4-fluoro-2,6-diisopropylphenyl)acetic acid (73 mg, 0.31 mmol), DCM (2 mL), and DMF (0.05 mL). This was followed by the addition of oxalyl chloride (0.5 mL) dropwise with stirring at RT. The solution was stirred for 30 min at RT and then was concentrated under vacuum. Into a 50-mL round-bottom flask was placed a solution of N′-(tert-butyldimethylsilyl)-4-(2-hydroxypropan-2-yl)benzenesulfonimidamide (100 mg, 0.30 mmol) in THF (3 mL). This was followed by the addition of NaH (60% wt., 42 mg, 1.04 mmol) in portions at 0° C. The solution was stirred for 5 min at RT. Then to the above was added the solution of 2-(4-fluoro-2,6-diisopropylphenyl)acetyl chloride in THF (1 mL) prepared as shown above. The resulting solution was stirred for 1 h at RT, after which it was quenched by the addition of 5 mL of water and extracted with 2×5 mL of ethyl acetate. The combined organic layers were dried over anhydrous Na₂SO₄, and concentrated under vacuum. This resulted in 159 mg (96%) of the title compound as yellow crude oil. MS-ESI: 547.3 (M−1).

### Step 2: 2-(4-Fluoro-2,6-diisopropylphenyl)-N-(4-(2-hydroxypropan-2-yl)phenylsulfonimidoyl)acetamide

Into a 50-mL round-bottom flask was placed a solution of N-(tert-butyldimethylsilylamino-4-(2-hydroxylpropan-2-yl)phenylhydrosulfonimidoyl)-2-(4-fluoro-2,6-diisopropylphenyl)acetamide (159 mg, 0.29 mmol) in DCM (10 mL). Then TFA (0.2 mL) was added. The resulting solution was stirred for 1 h at RT and was concentrated under vacuum after that. The crude product was purified by Prep-HPLC using method E eluted with a gradient of 26-50% ACN. This resulted in 13.0 mg (10%) of Example 1 as a white solid. MS-ESI: 435.3 (M+1). ¹H NMR (300 MHz, DMSO-d₆) δ 7.75 (d, J=8.7 Hz, 2H), 7.61 (d, J=8.7 Hz, 2H), 7.57 (s, 2H), 6.82 (d, 0.1=10.5 Hz, 2H), 5.21 (s, 1H), 3.62-3.54 (m, 2H) 3.07-2.98 (m, 2H), 1.41 (s, 6H), 1.13 (d, J=6.9 Hz, 6H), 1.08 (d, J=6.9 Hz, 6H).

### Step 3: Chiral Separation

The product obtained as described in the previous step (90 mg) was resolved by Chiral-Prep-HPLC using the following conditions: Column, ChiralPak ID, 2*25 cm, 5 um; mobile phase, Hex and IPA (hold 40% IPA over 16 min); Flow rate, 20 mL/min; Detector, UV 254/220 nm. This resulted in 16.0 mg (front peak, enantiomer 1, 99% ee) of Example 2 as a white solid and 44.8 mg (second peak, enantiomer 2, 99% ee) of Example 3 as a light yellow solid. Absolute stereochemistry of these two isomers has not been assigned.

Example 2: MS-ESI: 435.1 (M+1). ¹H NMR (300 MHz, DMSO-d₆) δ 7.75 (d, J=8.4 Hz, 2H), 7.61 (d, J=9.0 Hz, 4H), 6.82 (d, J=10.5 Hz, 2H), 5.18 (s, 1H), 3.62-3.58 (m, 2H), 3.07-2.98 (m, 2H), 1.41 (s, 6H), 1.08 (d, J=6.9 Hz, 6H), 1.00 (d, J=6.6 Hz, 6H).

Example 3: MS-ESI: 435.1 (M+1). ¹H NMR (300 MHz, DMSO-d₆) δ 7.74 (d, J=8.7 Hz, 2H), 7.57 (d, J=8.4 Hz, 4H), 6.81 (d, J=10.5 Hz, 2H), 5.21 (s, 1H), 3.58-3.57 (m, 2H), 3.09-3.02 (m, 2H), 1.41 (s, 6H), 1.07 (d, J=6.6 Hz, 6H), 1.01 (d, J=6.6 Hz, 6H).

### Example 4 (129)

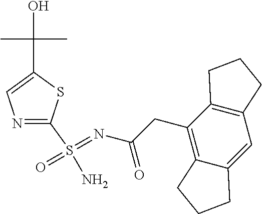 [SMILES: CC(C)(O)c1cnc(S(N)(=O)=NC(=O)Cc2c3c(cc4c2CCC4)CCC3)s1]

### 2-(1,2,3,5,6,7-Hexahydro-s-indacen-4-yl)-N-(5-(2-hydroxypropan-2-yl)thiazol-2-ylsulfonimidoyl)acetamide (Scheme 2)

### Examples 5 (129b) and 6 (129a)

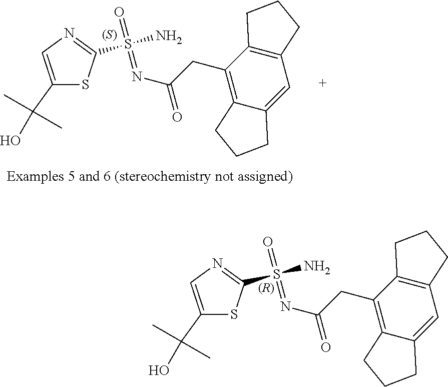

### (S)- and (R)-2-(1,2,3,5,6,7-Hexahydro-s-indacen-4-yl)-N-(5-(2-hydroxypropan-2-yl)thiazol-2-ylsulfonimidoyl)acetamide

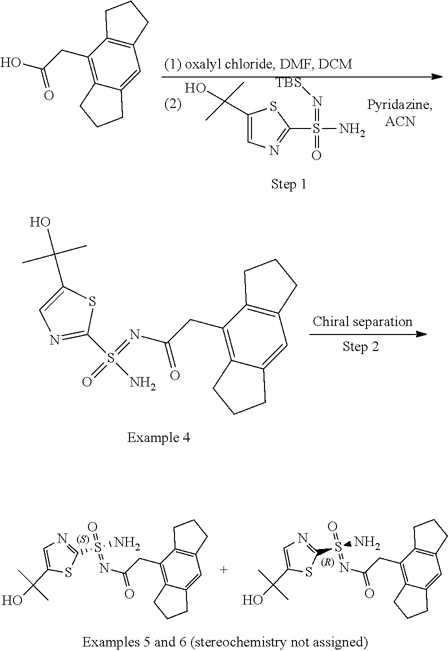

### Step 1: 2-(1,2,3,5,6,7-Hexahydro-s-indacen-4-yl)-N-(5-(2-hydroxypropan-2-yl)thiazol-2-ylsulfonimidoyl)acetamide

Into a 50-mL round-bottom flask was placed 2-(1,2,3,5,6,7-hexahydro-s-indacen-4-yl)acetic acid (100 mg, 0.46 mmol), DCM (2 mL), and DMF (0.05 mL). This was followed by the addition of oxalyl chloride (0.5 mL) dropwise with stirring at RT. The solution was stirred for 30 min at RT and then was concentrated under vacuum. The above mixture was diluted in ACN (3 mL). This was followed by the addition of a solution of pyridazine (37 mg, 0.46 mmol) in ACN (1 mL). The solution was stirred for 1 min at RT and then a solution of N′-(tert-butyldimethylsilyl)-5-(2-hydroxypropan-2-yl)thiazole-2-sulfonimidamide (154 mg, 0.46 mmol) in ACN (2 mL) was added. The resulting solution was stirred for 2 h at RT, after which it was concentrated under vacuum. The resulting residue was applied onto a silica gel column and eluted with ethyl acetate/petroleum ether (1:2 to 1:1). The crude product was purified by Prep-HPLC using method E eluted with a gradient of 26-50% ACN. This resulted in 10 mg (5%) of Example 4 as a white solid. MS-ESI: 420.2 (M+1). ¹H NMR (300 MHz, DMSO-d₆) δ 7.84 (br s, 2H), 7.72 (s, 1H), 6.89 (s, 1H), 5.85 (s, 1H), 3.48-3.37 (m, 2H), 2.80-2.70 (m, 8H), 1.99-1.90 (m, 4H), 1.52-1.51 (m, 6H).

### Step 2: Chiral Separation

The product obtained as described in the previous step (40 mg) was resolved by Chiral-Prep-HPLC using the following conditions: ChiralPak IG, 2*25 cm, 5 um; mobile phase, Hex (0.1% TFA) and EtOH (hold 30% EtOH over 13.5 min); Flow rate, 20 m/min; Detector, UV 254/220 nm. This resulted in 15.3 mg (front peak, enantiomer 1, 100% ee) of Example 5 as a white solid and 14.4 mg (second peak, enantiomer 2, 100% ee) of Example 6 as a white solid. Absolute stereochemistry of these two isomers has not been assigned.

Example 5: MS-ESI: 420.2 (M+1). ¹H NMR (300 MHz, DMSO-d₆) δ 8.12 (s, 2H), 7.80 (s, 1H), 6.90 (s, 1H), 5.93 (s, 1H), 3.48-3.40 (m, 2H), 2.80-2.50 (m, 8H), 2.08-1.89 (m, 4H), 1.54-1.52 (m, 6H).

Example 6: MS-ESI: 420.2 (M+1). ¹H NMR (300 MHz, DMSO-d₆) δ 8.12 (s, 2H), 7.80 (s, 1H), 6.91 (s, 1H), 5.93 (s, 1H), 3.48-3.40 (m, 2H), 2.80-2.69 (m, 8H), 1.99-1.90 (m, 4H), 1.54-1.52 (m, 6H).

### Example 7 (132)

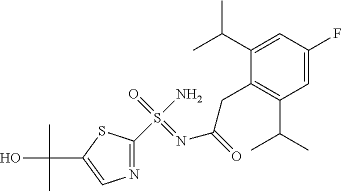 [SMILES: CC(C)c1cc(F)cc(C(C)C)c1CC(=O)N=S(N)(=O)c1ncc(C(C)(C)O)s1]

### 2-(4-Fluoro-2,6-diisopropylphenyl)-N-(5-(2-hydroxypropan-2-yl)thiazole-2-sulfonimidoyl)acetamide (Scheme 3)

### Examples 8 (132b) and 9 (132a)

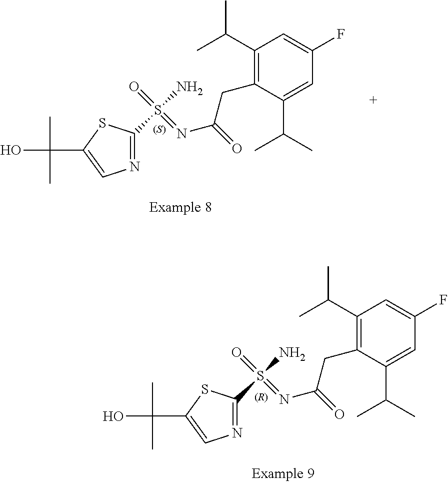

### (S)- and (R)-2-(4-fluoro-2,6-diisopropylphenyl)-N-(5-(2-hydroxypropan-2-yl)thiazole-2-sulfonimidoyl)acetamide

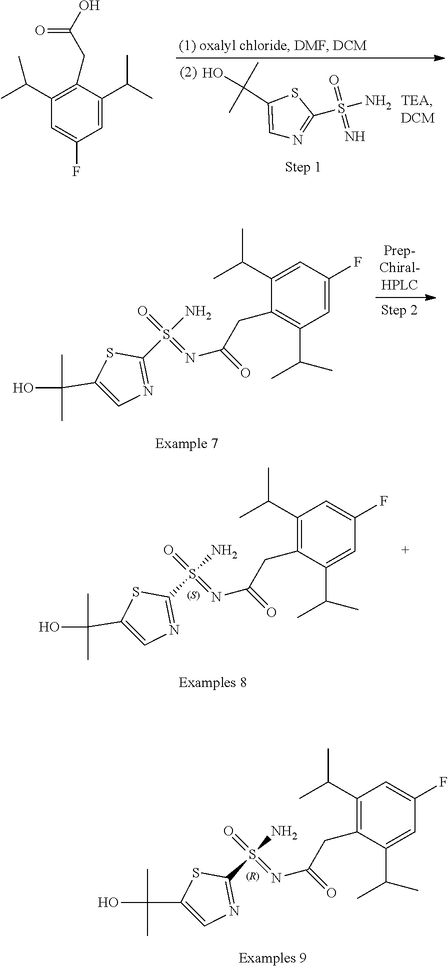

### Step 1: 2-(4-Fluoro-2,6-diisopropylphenyl)-N-(5-(2-hydroxypropan-2-yl)thiazole-2-sulfonimidoyl)acetamide

Into a 50-mL round-bottom flask was placed 2-(4-fluoro-2,6-diisopropylphenyl)acetic acid (108 mg, 0.45 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (1 mL), was added to a solution of 5-(2-hydroxypropan-2-yl)thiazole-2-sulfonimidamide (60 mg, 0.27 mmol) and TEA (150 mg, 1.48 mmol) in DCM (3 mL) dropwise with stirring at RT. The resulting solution was stirred for 1 h at RT and was then concentrated under vacuum. The crude product was purified by Prep-HPLC using method E eluted with a gradient of 22-44% ACN. This resulted in 2.1 mg (1%) of Example 7 as a white solid. MS-ESI: 442.3 (M+1). ¹H NMR (300 MHz, CD₃OD-d₄) δ 7.67 (s, 1H), 6.75 (d, J=10.5 Hz, 2H), 3.74 (s, 2H), 3.17-3.03 (m, 2H), 1.58 (s, 6H), 1.12 (d, J=6.8 Hz, 6H), 1.10 (d, J=6.8 Hz, 6H).

### Step 2: Chiral Separation

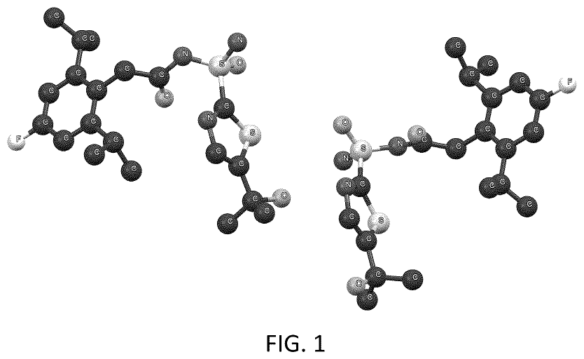

The product obtained in the previous step (10 mg) was resolved by Chiral-Prep-HPLC using following conditions: Column, ChiralPak ID, 2*25 cm, 5 um; mobile phase, Hex (0.1% FA) and EtOH (hold 20% EtOH in over 10 min); Flow rate, 20 mL/min; Detector, UV 254/220 nm. This resulted in 3.6 mg (front peak, enantiomer 1, 99% ee) of Example 8 as a yellow solid and 3.1 mg (second peak, enantiomer 2, 99% ee) of Example 9 as a yellow solid. Single crystal X-ray crystallographic analysis was performed on compound 132b. Table N below shows fractional atomic coordinates of compound 132b, and FIG. 1 shows ball and stick models of the asymmetrical unit containing two crystallographically independent molecules of compound 132b, with hydrogen atoms omitted for clarity. Based on these results, the absolute stereochemistry of compound 132b was assigned as (S) by single crystal X-ray structure determination. Therefore, compound 132a was assigned as the (R) isomer.

Example 8: MS-ESI: 442.2 (M+1). ¹H NMR (300 MHz, DMSO-d₆) δ 8.21 (br s, 2H), 7.75 (s, 1H), 6.84 (d, J=10.5 Hz, 2H), 5.88 (s, 1H), 3.65-3.63 (m, 2H), 3.10-2.97 (m, 2H), 1.51 (s, 6H), 1.09 (d, J=6.9 Hz, 6H), 1.04 (d, J=6.9 Hz, 6H).

Example 9: MS-ESI: 442.2 (M+1). ¹H NMR (300 MHz, DMSO-d₆) δ 7.73 (s, 1H), 6.83 (d, J=10.5 Hz, 2H), 5.87 (s, 1H), 3.63-3.59 (m, 2H), 3.10-3.03 (m, 2H), 1.51 (s, 6H), 1.09 (d, J=6.9 Hz, 6H), 1.04 (d, J=6.6 Hz, 6H).

TABLE N
Fractional Atomic Coordinates (×10⁴) and Equivalent Isotropic
Displacement Parameters (Å² × 10³) for Example 8. U_{eq} is defined as ⅓
of the trace of the orthogonalised U_{IJ} tensor.

| Atom | x | | y | | z | | U(eq) | |
|---|---|---|---|---|---|---|---|---|
| S3 | 6096 | (2) | 1631.9 | (12) | 6214.0 | (7) | 43.8 | (3) |
| S1 | 4186 | (3) | 6312.8 | (13) | 3775.3 | (8) | 49.4 | (4) |
| S4 | 7832 | (3) | 4132.7 | (15) | 5650.5 | (8) | 53.7 | (4) |
| S2 | 4909 | (3) | 9172.1 | (15) | 3606.7 | (9) | 64.2 | (5) |
| O4 | 7737 | (8) | 1198 | (4) | 5669 | (2) | 56.1 | (11) |
| O5 | 9907 | (8) | 1617 | (5) | 7085 | (2) | 62.4 | (12) |
| O1 | 2799 | (9) | 5566 | (4) | 4214 | (2) | 64.9 | (13) |
| F2 | 14863 | (9) | 1324 | (5) | 9826 | (3) | 88.3 | (14) |
| N4 | 6228 | (9) | 1015 | (5) | 6933 | (2) | 47.7 | (11) |
| O2 | 102 | (9) | 7078 | (6) | 2977 | (2) | 70.3 | (14) |
| F1 | −4874 | (11) | 7107 | (6) | 161 | (3) | 100.7 | (17) |
| N5 | 3489 | (10) | 1470 | (6) | 6026 | (3) | 54.6 | (13) |
| O3 | 2378 | (9) | 12418 | (4) | 4594 | (2) | 60.3 | (12) |
| N3 | 1905 | (9) | 8406 | (5) | 4478 | (2) | 46.5 | (11) |
| N1 | 4115 | (10) | 6318 | (5) | 3000 | (3) | 55.1 | (13) |
| N2 | 6903 | (11) | 5865 | (6) | 3894 | (3) | 60.1 | (15) |
| N6 | 5327 | (10) | 4040 | (5) | 6756 | (3) | 58.1 | (14) |
| O6 | 8678 | (13) | 6628 | (6) | 5147 | (3) | 86.0 | (18) |
| C3 | 3284 | (10) | 10337 | (6) | 4107 | (3) | 45.2 | (13) |
| C27 | 8106 | (11) | 1236 | (5) | 7308 | (3) | 46.2 | (14) |
| C29 | 9664 | (12) | 1054 | (6) | 8500 | (3) | 50.9 | (15) |
| C32 | 13181 | (13) | 1229 | (7) | 9385 | (3) | 59.3 | (17) |
| C24 | 7970 | (11) | 6826 | (6) | 5836 | (3) | 50.5 | (14) |
| C6 | 6192 | (12) | 11937 | (7) | 4004 | (4) | 63.5 | (18) |
| C30 | 10056 | (13) | 2297 | (6) | 8708 | (3) | 55.4 | (16) |
| C22 | 5791 | (13) | 5307 | (6) | 6651 | (3) | 59.0 | (17) |
| C28 | 7637 | (12) | 955 | (6) | 8054 | (3) | 51.2 | (15) |
| C23 | 7090 | (11) | 5560 | (6) | 6085 | (3) | 46.0 | (14) |
| C1 | 3454 | (11) | 7995 | (6) | 4013 | (3) | 46.6 | (13) |
| C2 | 1819 | (11) | 9753 | (6) | 4535 | (3) | 48.0 | (13) |
| C34 | 11159 | (13) | −85 | (6) | 8717 | (3) | 55.8 | (16) |
| C9 | 415 | (13) | 7159 | (7) | 1501 | (3) | 57.2 | (17) |
| C31 | 11805 | (14) | 2368 | (7) | 9156 | (4) | 63.5 | (18) |
| C10 | −915 | (15) | 8349 | (7) | 1276 | (4) | 65.2 | (19) |
| C33 | 12915 | (14) | 27 | (7) | 9168 | (3) | 60.3 | (17) |
| C4 | 3585 | (11) | 11787 | (6) | 4018 | (3) | 46.4 | (14) |
| C7 | 2090 | (13) | 6835 | (7) | 2692 | (3) | 55.1 | (16) |
| C8 | 2514 | (14) | 7140 | (8) | 1955 | (3) | 64.7 | (19) |
| C13 | −1943 | (16) | 5980 | (8) | 843 | (4) | 71 | (2) |
| C12 | −3143 | (15) | 7121 | (8) | 616 | (4) | 70 | (2) |
| C14 | −148 | (15) | 5971 | (7) | 1295 | (4) | 67 | (2) |
| C38 | 8604 | (16) | 3579 | (7) | 8461 | (4) | 73 | (2) |
| C5 | 2485 | (14) | 12376 | (7) | 3372 | (4) | 64.0 | (18) |
| C35 | 10957 | (16) | −1446 | (7) | 8477 | (4) | 72 | (2) |
| C11 | −2668 | (16) | 8307 | (8) | 821 | (4) | 76 | (2) |
| C21 | 6299 | (10) | 3342 | (6) | 6257 | (3) | 45.8 | (14) |
| C39 | 10220 | (20) | 4381 | (9) | 8022 | (5) | 99 | (3) |
| C40 | 7385 | (19) | 4354 | (9) | 9037 | (6) | 101 | (3) |
| C15 | 1040 | (20) | 4634 | (9) | 1552 | (6) | 95 | (3) |
| C26 | 9960 | (20) | 7080 | (12) | 6237 | (6) | 120 | (5) |
| C18 | −480 | (20) | 9672 | (9) | 1490 | (5) | 98 | (3) |
| C37 | 10310 | (40) | −2330 | (12) | 9044 | (6) | 153 | (6) |
| C25 | 5915 | (17) | 7965 | (8) | 5857 | (6) | 92 | (3) |
| C17 | −680 | (30) | 3869 | (11) | 1918 | (6) | 132 | (5) |
| C16 | 2410 | (20) | 3878 | (10) | 979 | (8) | 124 | (5) |
| C19 | 80 | (40) | 10550 | (14) | 950 | (8) | 198 | (10) |
| C36 | 13120 | (30) | −2025 | (15) | 8091 | (10) | 199 | (10) |
| C20 | −2400 | (50) | 10316 | (15) | 1931 | (10) | 292 | (18) |

### Example 10 (134)

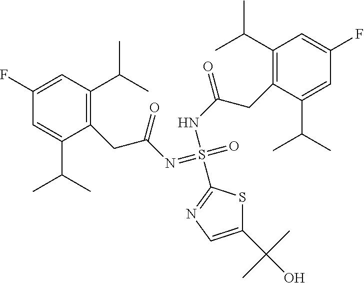 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1]

### N,N′-(5-(2-hydroxypropane-2-yl)thiazol-2-ylsulfinyl)bis 2-(4-fluoro-2,6-diisopropylphenyl)acetamide) (Scheme 4)

Into a 50-mL round-bottom flask was placed 2-(4-fluoro-2,6-diisopropylphenyl)acetic acid (242 mg, 1.02 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)thiazole-2-sulfonimidamide (220 mg, 0.99 mmol) and TEA (400 mg, 3.95 mmol) in DCM (3 mL) dropwise with stirring at RT. The resulting solution was stirred for 1 h at RT and was then concentrated under vacuum. The crude product was purified by Prep-HPLC using method E eluted with a gradient of 63˜65% ACN. This resulted in 51.6 mg (8%) of Example 10 as a white solid. MS-ESI: 660.5 (M−1). ¹H NMR (300 MHz, CD₃OD-d₄) δ 7.59 (s, 1H), 5.75 (d, J=10.2 Hz, 4H), 3.82 (s, 4H), 3.16-3.06 (m, 4H), 1.59 (s, 6H), 1.32-1.13 (m, 24H).

### Example 11 (137)

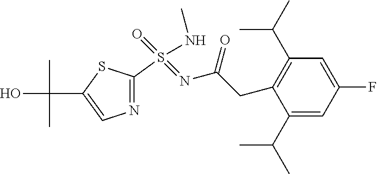 [SMILES: CNS(=O)(=NC(=O)Cc1c(C(C)C)cc(F)cc1C(C)C)c1ncc(C(C)(C)O)s1]

### 2-(4-Fluoro-2,6-diisopropylphenyl)-N-(5-(2-hydroxypropan-2-yl-N-methylthiazole-2-sulfonimidoyl)acetamide (Scheme 5)

Into a 50-mL round-bottom flask was placed 2-(4-fluoro-2,6-diisopropylphenyl)acetic acid (233 mg, 0.98 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)-N′-methylthiazole-2-sulfonimidamide (230 mg, 0.98 mmol) and TEA (400 mg, 3.95 mmol) in DCM (3 mL) dropwise with stirring at RT. The resulting solution was stirred for 1 h at RT and then was concentrated under vacuum. The crude product was purified by Prep-HPLC using method E eluted with a gradient of 20˜80% ACN. This resulted in 17.7 mg (4%) of Example 11 as a light yellow solid. MS-ESI: 456.2 (M+1). ¹H NMR (300 MHz, DMSO-d₆) major tautomer δ 8.34 (s, 1H), 7.85 (s, 1H), 6.86 (d, J=10.5 Hz, 2H), 5.94 (s, 1H), 3.71 (d, J=3.6 Hz, 2H), 3.14-3.02 (m, 2H), 2.55 (s, 3H), 1.52 (s, 6H), 1.13 (d, J=4.5 Hz, 6H), 1.09 (d, J=4.5 Hz, 6H).

### Example 12 (136)

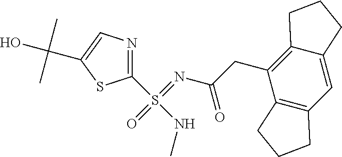 [SMILES: CNS(=O)(=NC(=O)Cc1c2c(cc3c1CCC3)CCC2)c1ncc(C(C)(C)O)s1]

### 2-(1,2,3,5,6,7-Hexahydro-s-indacen-4-yl)-N-(5-(2-hydroxypropan-2-yl)-N-methylthiazole-2-sulfonimidoyl)acetamide (Scheme 6)

Into a 50-mL round-bottom flask was placed 2-(1,2,3,5,6,7-hexahydro-s-indacen-4-yl)acetic acid (156 mg, 0.72 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)-N′-methylthiazole-2-sulfonimidamide (170 mg, 0.72 mmol) and DBU (370 mg, 2.43 mmol) in DCM (3 mL) dropwise with stirring at RT. The resulting solution was stirred for 2 h at RT and then was quenched by the addition of 10 mL of water. The resulting solution was extracted with 3×10 mL of ethyl acetate, dried over anhydrous Na₂SO₄, and then concentrated under vacuum. The crude product was purified by Prep-HPLC using method E eluted with a gradient of 5-15% ACN. This resulted in 11.3 mg (4%) of the title compound as a light yellow solid. MS-ESI: 434.3 (M+1). ¹H NMR (300 MHz, CD₃OD-d₄) major tautomer δ 7.75 (s, 1H), 6.89 (s, 1H), 3.56 (s, 2H), 2.83-2.73 (m, 8H), 2.61 (s, 3H), 2.06-1.95 (m, 4H), 1.59 (s, 6H).

TABLE 8
Example in the following table was prepared using similar conditions as described in Example 1 and Scheme 1
from appropriate starting materials.

| Example | Compound | | | LC-MS |
|---|---|---|---|---|
| # | Number | Structure | IUPAC Name | [M + H]⁺ |
| 13 | 133 | 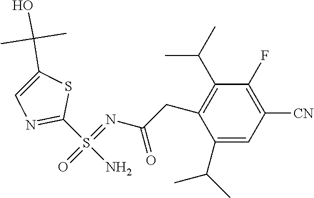 [SMILES: CC(C)c1cc(C#N)c(F)c(C(C)C)c1CC(=O)N=S(N)(=O)c1ncc(C(C)(C)O)s1] | 2-(4-cyano-3-fluoro-2,6- diisopropylphenyl)-N-(5-(2- hydroxypropan-2-yl)thiazol-2- ylsulfonimidoyl)acetamide | 467.2 |

TABLE 9
Examples in the following table were prepared using similar conditions as described in Example 4 and Scheme 2 from appropriate
starting materials.

| Example | Compound | | | LC-MS |
|---|---|---|---|---|
| # | Number | Structure | IUPAC Name | [M + H]⁺ |
| 14 | 141 | 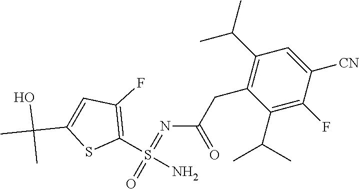 [SMILES: CC(C)c1cc(C#N)c(F)c(C(C)C)c1CC(=O)N=S(N)(=O)c1sc(C(C)(C)O)cc1F] | 2-(4-cyano-3-fluoro-2,6- diisopropylphenyl)-N-(3-fluoro-5- (2-hydroxypropan-2-yl)thiophen- 2-ylsulfonimidoyl)acetamide | 484.2 |
| 15 | 139 | 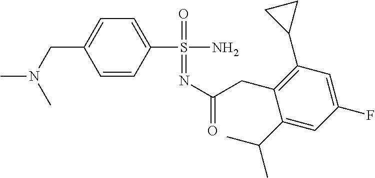 [SMILES: CC(C)c1cc(F)cc(C2CC2)c1CC(=O)N=S(N)(=O)c1ccc(CN(C)C)cc1] | 2-(2-cyclopropyl-4-fluoro-6- isopropylphenyl)-N-(4- ((dimethylamino)methyl)phenyl- sulfonimidoyl)acetamide | 432.2 |
| 16 | 138 | 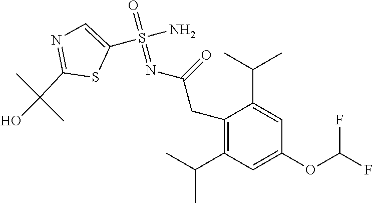 [SMILES: CC(C)c1cc(OC(F)F)cc(C(C)C)c1CC(=O)N=S(N)(=O)c1cnc(C(C)(C)O)s1] | 2-(4-(difluoromethoxy)-2,6- diisopropylphenyl)-N-(2-(2- hydroxypropan-2-yl)thiazol-5- ylsulfonimidoyl)acetamide | 490.3 |
| 17 | 140 | 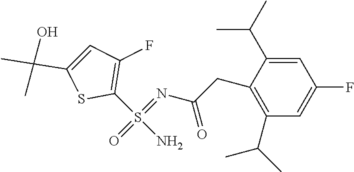 [SMILES: CC(C)c1cc(F)cc(C(C)C)c1CC(=O)N=S(N)(=O)c1sc(C(C)(C)O)cc1F] | 2-(4-fluoro-2,6- diisopropylphenyl)-N-(3-fluoro-5- (2-hydroxypropan-2-yl)thiophen- 2-ylsulfonimidoyl)acetamide | 459.2 |
| 18 | 142 | 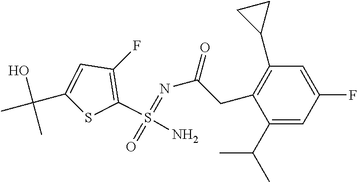 [SMILES: CC(C)c1cc(F)cc(C2CC2)c1CC(=O)N=S(N)(=O)c1sc(C(C)(C)O)cc1F] | 2-(2-cyclopropyl-4-fluoro-6- isopropylphenyl)-N-(3-fluoro-5- (2-hydroxypropan-2-yl)thiophen- 2-ylsulfonimidoyl)acetamide | 457.2 |
| 19 | 143 | 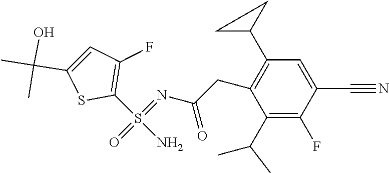 [SMILES: CC(C)c1c(F)c(C#N)cc(C2CC2)c1CC(=O)N=S(N)(=O)c1sc(C(C)(C)O)cc1F] | 2-(4-cyano-6-cyclopropyl-3- fluoro-2-isopropylphenyl)-N-(3- fluoro-5-(2-hydroxypropan-2- yl)thiophen-2- ylsulfonimidoyl)acetamide | 482.2 |
| 20 | 197 | 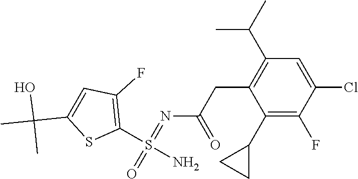 [SMILES: CC(C)c1cc(Cl)c(F)c(C2CC2)c1CC(=O)N=S(N)(=O)c1sc(C(C)(C)O)cc1F] | 2-(4-chloro-2-cyclopropyl-3- fluoro-6-isopropylphenyl)-N-(3- fluoro-5-(2-hydroxypropan-2- yl)thiophen-2- ylsulfonimidoyl)acetamide | 491.2 |
| 21 | 101 | 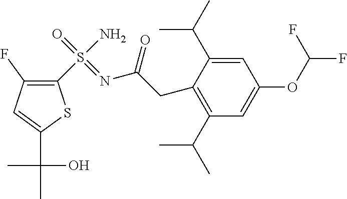 [SMILES: CC(C)c1cc(OC(F)F)cc(C(C)C)c1CC(=O)N=S(N)(=O)c1sc(C(C)(C)O)cc1F] | 2-(4-(difluoromethoxy)-2,6- diisopropylphenyl)-N-(3-fluoro-5- (2-hydroxypropan-2-yl)thiophen- 2-ylsulfonimidoyl)acetamide | 507.2 |
| 22 | 152 | 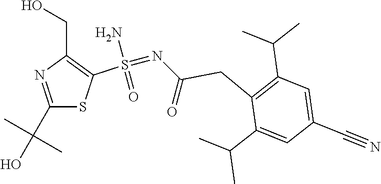 [SMILES: CC(C)c1cc(C#N)cc(C(C)C)c1CC(=O)N=S(N)(=O)c1sc(C(C)(C)O)nc1CO] | 2-(4-cyano-2,6- diisopropylphenyl)-N-(4- (hydroxymethyl)-2-(2- hydroxypropan-2-yl)thiazol-5- ylsulfonimidoyl)acetamide | 479.2 |
| 23 | 150 | 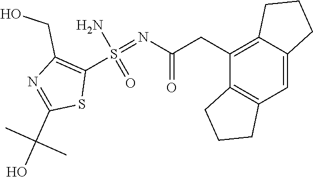 [SMILES: CC(C)(O)c1nc(CO)c(S(N)(=O)=NC(=O)Cc2c3c(cc4c2CCC4)CCC3)s1] | 2-(1,2,3,5,6,7-hexahydros- indacen-4-yl)-N-(4- (hydroxymethyl)-2-(2- hydroxypropan-2-yl)thiazol-5- ylsulfonimidoyl)acetamide | 450.2 |
| 24 | 148 | 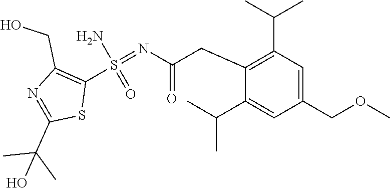 [SMILES: COCc1cc(C(C)C)c(CC(=O)N=S(N)(=O)c2sc(C(C)(C)O)nc2CO)c(C(C)C)c1] | 2-(2,6-diisopropyl-4- (methoxymethyl)phenyl)-N-(4- (hydroxymethyl)-2-(2- hydroxypropan-2-yl)thiazole-5- sulfonimidoyl)acetamide | 498.2 |
| 25 | 147 | 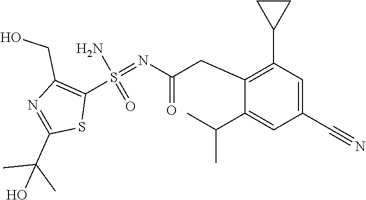 [SMILES: CC(C)c1cc(C#N)cc(C2CC2)c1CC(=O)N=S(N)(=O)c1sc(C(C)(C)O)nc1CO] | 2-(4-cyano-2-cyclopropyl-6- isopropylphenyl)-N-(4- (hydroxymethyl)-2-(2- hydroxypropan-2-yl)thiazole-5- sulfonimidoyl)acetamide | 477.2 |
| 26 | 113 | 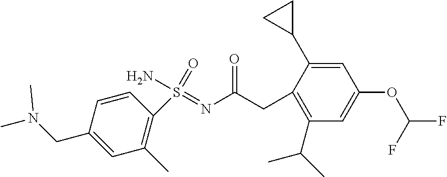 [SMILES: Cc1cc(CN(C)C)ccc1S(N)(=O)=NC(=O)Cc1c(C(C)C)cc(OC(F)F)cc1C1CC1] | 2-(2-cyclopropyl-4- (difluoromethoxy)-6- isopropylphenyl)-N-(4- ((dimethylamino)methyl)-2- methylphenylsulfonimidoyl) acetamide | 494.2 |
| 27 | 151 | 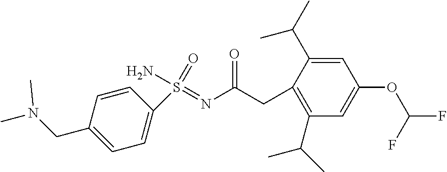 [SMILES: CC(C)c1cc(OC(F)F)cc(C(C)C)c1CC(=O)N=S(N)(=O)c1ccc(CN(C)C)cc1] | 2-(4-(difluoromethoxy)-2,6- diisopropylphenyl)-N-(4- ((dimethylamino)methyl)phenyl- sulfonimidoyl)acetamide | 482.2 |
| 28 | 114 | 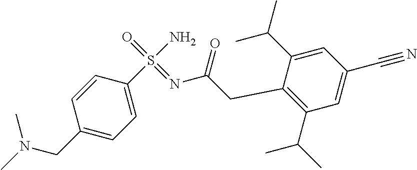 [SMILES: CC(C)c1cc(C#N)cc(C(C)C)c1CC(=O)N=S(N)(=O)c1ccc(CN(C)C)cc1] | 2-(4-cyano-2,6- diisopropylphenyl)-N-(4- ((dimethylamino)methyl)phenyl- sulfonimidoyl)acetamide | 441.2 |
| 29 | 112 | 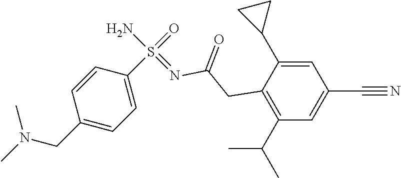 [SMILES: CC(C)c1cc(C#N)cc(C2CC2)c1CC(=O)N=S(N)(=O)c1ccc(CN(C)C)cc1] | 2-(4-cyano-2-cyclopropyl-6- isopropylphenyl)-N-(4- ((dimethylamino)methyl)phenyl- sulfonimidoyl)acetamide | 439.2 |
| 30 | 116 | 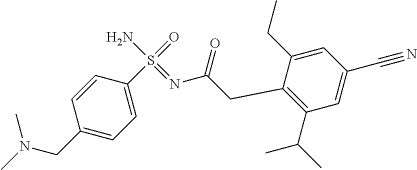 [SMILES: CCc1cc(C#N)cc(C(C)C)c1CC(=O)N=S(N)(=O)c1ccc(CN(C)C)cc1] | 2-(4-cyano-2-ethyl-6- isopropylphenyl)-N-(4- ((dimethylamino)methyl)phenyl- sulfonimidoyl)acetamide | 427.2 |
| 31 | 105 | 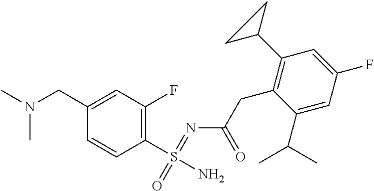 [SMILES: CC(C)c1cc(F)cc(C2CC2)c1CC(=O)N=S(N)(=O)c1ccc(CN(C)C)cc1F] | 2-(2-cyclopropyl-4-fluoro-6- isopropylphenyl)-N-(4- ((dimethylamino)methyl)-2- fluorophenylsulfonimidoyl) acetamide | 450.2 |
| 32 | 117 | 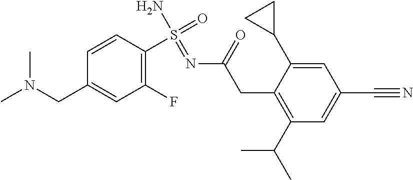 [SMILES: CC(C)c1cc(C#N)cc(C2CC2)c1CC(=O)N=S(N)(=O)c1ccc(CN(C)C)cc1F] | 2-(4-cyano-2-cyclopropyl-6- isopropylphenyl)-N-(4- ((dimethylamino)methyl)-2- fluorophenylsulfonimidoyl) acetamide | 457.2 |
| 33 | 109 | 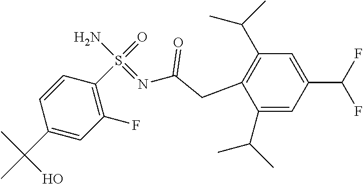 [SMILES: CC(C)c1cc(C(F)F)cc(C(C)C)c1CC(=O)N=S(N)(=O)c1ccc(C(C)(C)O)cc1F] | 2-(4-(difluoromethyl)-2,6- diisopropylphenyl)-N-(2-fluoro-4- (2-hydroxypropan-2- yl)phenylsulfonimidoyl)acetamide | 485.2 |
| 34 | 146 | 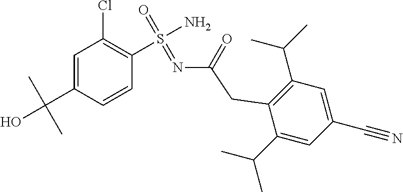 [SMILES: CC(C)c1cc(C#N)cc(C(C)C)c1CC(=O)N=S(N)(=O)c1ccc(C(C)(C)O)cc1Cl] | N-(2-chloro-4-(2-hydroxypropan- 2-yl)phenylsulfonimidoyl)-2-(4- cyano-2,6- diisopropylphenyl)acetamide | 476.2 |
| 35 | 110 | 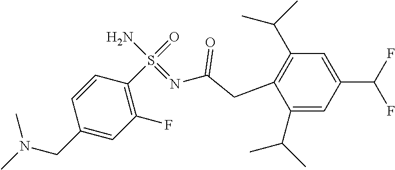 [SMILES: CC(C)c1cc(C(F)F)cc(C(C)C)c1CC(=O)N=S(N)(=O)c1ccc(CN(C)C)cc1F] | 2-(4-(difluoromethyl)-2,6- diisopropylphenyl)-N-(4- ((dimethylamino)methyl)-2- fluorophenylsulfonimidoyl) acetamide | 484.2 |
| 36 | 126 | 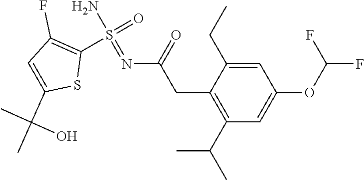 [SMILES: CCc1cc(OC(F)F)cc(C(C)C)c1CC(=O)N=S(N)(=O)c1sc(C(C)(C)O)cc1F] | 2-(4-(difluoromethoxy)-2-ethyl-6- isopropylphenyl)-N-(3-fluoro-5- (2-hydroxypropan-2-yl)thiophene- 2-sulfonimidoyl)acetamide | 493.1 |
| 37 | 104 | 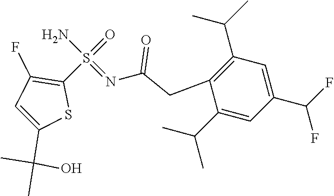 [SMILES: CC(C)c1cc(C(F)F)cc(C(C)C)c1CC(=O)N=S(N)(=O)c1sc(C(C)(C)O)cc1F] | 2-(4-(difluoromethyl)-2,6- diisopropylphenyl)-N-(3-fluoro-5- (2-hydroxypropan-2-yl)thiophene- 2-sulfonimidoyl)acetamide | 491.2 |
| 38 | 130 | 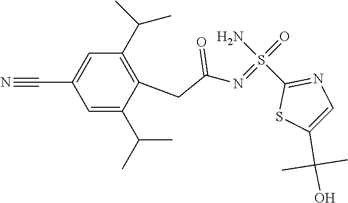 [SMILES: CC(C)c1cc(C#N)cc(C(C)C)c1CC(=O)N=S(N)(=O)c1ncc(C(C)(C)O)s1] | 2-(4-cyano-2,6- diisopropylphenyl)-N-(5-(2- hydroxypropan-2-yl)thiazole-2- sulfonimidoyl)acetamide | 449.2 |
| 39 | 107 | 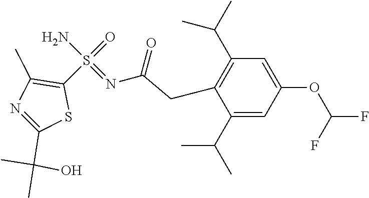 [SMILES: Cc1nc(C(C)(C)O)sc1S(N)(=O)=NC(=O)Cc1c(C(C)C)cc(OC(F)F)cc1C(C)C] | 2-(4-(difluoromethoxy)-2,6- diisopropylphenyl)-N-(2-(2- hydroxypropan-2-yl)-4- methylthiazole-5- sulfonimidoyl)acetamide | 504.2 |
| 40 | 106 | 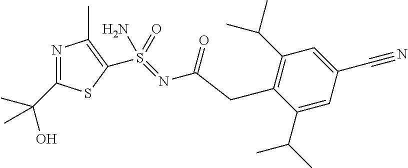 [SMILES: Cc1nc(C(C)(C)O)sc1S(N)(=O)=NC(=O)Cc1c(C(C)C)cc(C#N)cc1C(C)C] | 2-(4-cyano-2,6- diisopropylphenyl)-N-(2-(2- hydroxypropan-2-yl)-4- methylthiazole-5- sulfonimidoyl)acetamide | 463.2 |

TABLE 10
Example in the following table was prepared using similar conditions as described in Example 11 and Scheme 5 from
appropriate starting materials.

| Example | Compound | | | LC-MS |
|---|---|---|---|---|
| # | Number | Structure | IUPAC Name | [M + H]⁺ |
| 41 | 135 | 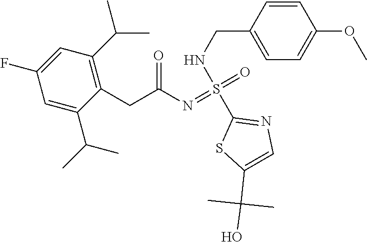 [SMILES: COc1ccc(CNS(=O)(=NC(=O)Cc2c(C(C)C)cc(F)cc2C(C)C)c2ncc(C(C)(C)O)s2)cc1] | 2-(4-fluoro-2,6- diisopropylphenyl)-N-(5-(2- hydroxypropan-2-yl)-N-(4- methoxybenzyl)thiazole-2- sulfonimidoyl)acetamide | 562.5 |

TABLE 11
Examples in the following table were obtained from chiral HPLC resolutions of racemic examples described above. The chiral column and eluents
are listed in the table. As a convention, the faster-eluting enantiomer is always listed first in the table followed by the slower-eluting enantiomer
of the pair. The symbol * at a chiral center denotes that this chiral center has been resolved and the absolute stereochemistry at that center
has not been determined.

| | Com- | | | | | |
|---|---|---|---|---|---|---|
| Ex. | pound | | | | | LC-MS |
| # | Number | Structure | IUPAC Name | Column | Eluents | [M + H]⁺ |
| 42 | 133b or 133a | 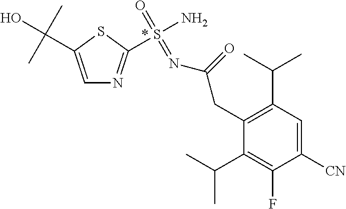 [SMILES: CC(C)c1cc(C#N)c(F)c(C(C)C)c1CC(=O)N=S(N)(=O)c1ncc(C(C)(C)O)s1] | (S)-or (R)- 2-(4-cyano-3-fluoro-2,6- diisopropylphenyl)-N-(5-(2- hydroxypropan-2-yl)thiazol-2- ylsulfonimidoyl) acetamide | ChiralPak IC, 2*25 cm, 5 um | 50% IPA in CO₂ | 467.2 |
| 43 | 133a or 133b | 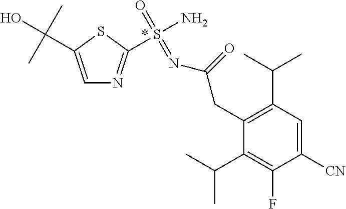 [SMILES: CC(C)c1cc(C#N)c(F)c(C(C)C)c1CC(=O)N=S(N)(=O)c1ncc(C(C)(C)O)s1] | (R)- or (S)- 2-(4-cyano-3-fluoro-2,6- diisopropylphenyl)-N-(5-(2- hydroxypropan-2-yl)thiazol-2- ylsulfonimidoyl) acetamide | ChiralPak IC, 2*25 cm, 5 um | 50% IPA in CO₂ | 467.2 |
| 44 | 141b or 141a | 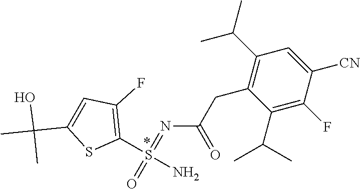 [SMILES: CC(C)c1cc(C#N)c(F)c(C(C)C)c1CC(=O)N=S(N)(=O)c1sc(C(C)(C)O)cc1F] | (S)- or (R)- 2-(4-cyano-3-fluoro-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% EtOH in Hex (1% TFA) | 484.2 |
| 45 | 141a or 141b | 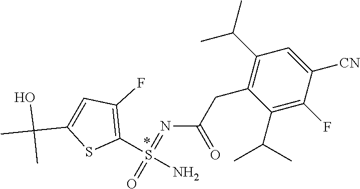 [SMILES: CC(C)c1cc(C#N)c(F)c(C(C)C)c1CC(=O)N=S(N)(=O)c1sc(C(C)(C)O)cc1F] | (R)- or (S)- 2-(4-cyano-3-fluoro-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% EtOH in Hex (1% TFA) | 484.2 |
| 46 | 139b or 139a | 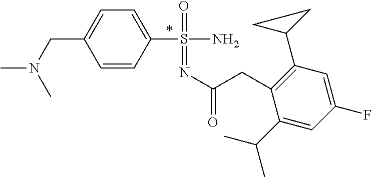 [SMILES: CC(C)c1cc(F)cc(C2CC2)c1CC(=O)N=S(N)(=O)c1ccc(CN(C)C)cc1] | (S)- or (R)- 2-(2-cyclopropyl-4-fluoro-6- isopropylphenyl)-N-(4- ((dimethylamino)methyl)phenyl- sulfonimidoyl)acetamide | ChiralPak IC, 2*25 cm, 5 um | 15% EtOH in Hex | 432.1 |
| 47 | 139a or 139b | 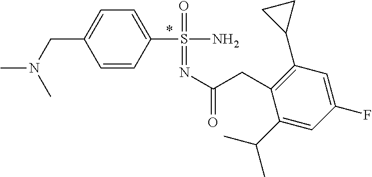 [SMILES: CC(C)c1cc(F)cc(C2CC2)c1CC(=O)N=S(N)(=O)c1ccc(CN(C)C)cc1] | (R)- or (S)- 2-(2-cyclopropyl-4-fluoro-6- isopropylphenyl)-N-(4- ((dimethylamino)methyl)phenyl- sulfonimidoyl)acetamide | ChiralPak IC, 2*25 cm, 5 um | 15% EtOH in Hex | 432.1 |
| 48 | 138b or 138a | 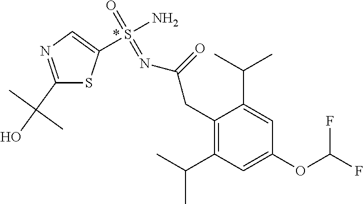 [SMILES: CC(C)c1cc(OC(F)F)cc(C(C)C)c1CC(=O)N=S(N)(=O)c1cnc(C(C)(C)O)s1] | (S)- or (R)- 2-(4-(difluoromethoxy)-2,6- diisopropylphenyl)-N-(2-(2- hydroxypropan-2-yl)thiazol-5- ylsulfonimidoyl)acetamide | ChiralPak IG, 2*25 cm, 5 um | 8% IPA in Hex (1% TFA) | 490.1 |
| 49 | 138a or 138b | 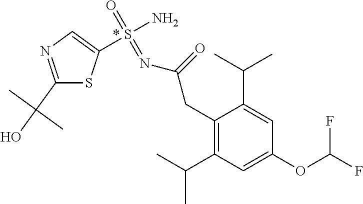 [SMILES: CC(C)c1cc(OC(F)F)cc(C(C)C)c1CC(=O)N=S(N)(=O)c1cnc(C(C)(C)O)s1] | (R)- or (S)- 2-(4-(difluoromethoxy)-2,6- diisopropylphenyl)-N-(2-(2- hydroxypropan-2-yl)thiazol-5- ylsulfonimidoyl)acetamide | ChiralPak IG, 2*25 cm, 5 um | 8% IPA in Hex (1% TFA) | 490.1 |
| 50 | 140b or 140a | 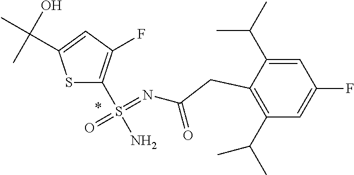 [SMILES: CC(C)c1cc(F)cc(C(C)C)c1CC(=O)N=S(N)(=O)c1sc(C(C)(C)O)cc1F] | (S)- or (R)- 2-(4-fluoro-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% IPA in Hex (0.1% DEA) | 459.2 |
| 51 | 140a or 140b | 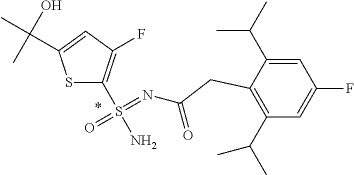 [SMILES: CC(C)c1cc(F)cc(C(C)C)c1CC(=O)N=S(N)(=O)c1sc(C(C)(C)O)cc1F] | (R)- or (S)- 2-(4-fluoro-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% IPA in Hex (0.1% DEA) | 459.2 |
| 52 | 144b or 144a | 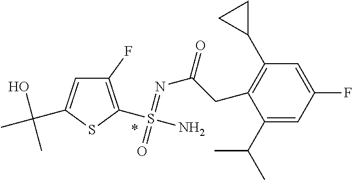 [SMILES: CC(C)c1cc(F)cc(C2CC2)c1CC(=O)N=S(N)(=O)c1sc(C(C)(C)O)cc1F] | (S)- or (R)- 2-(2-cyclopropyl-4-fluoro-6- isopropylphenyl)-N-(3-fluoro-5-(2- hydroxypropan-2-yl)thiophen-2- ylsulfonimidoyl)acetamide | ChiralPak ID, 2*25 cm, 5 um | 30% IPA in Hex | 457.2 |
| 53 | 144a or 144b | 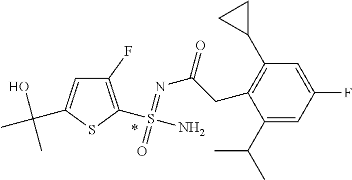 [SMILES: CC(C)c1cc(F)cc(C2CC2)c1CC(=O)N=S(N)(=O)c1sc(C(C)(C)O)cc1F] | (R)- or (S)- 2-(2-cyclopropyl-4-fluoro-6- isopropylphenyl)-N-(3-fluoro-5-(2- hydroxypropan-2-yl)thiophen-2- ylsulfonimidoyl)acetamide | ChiralPak ID, 2*25 cm, 5 um | 30% IPA in Hex | 457.2 |
| 54 | 145b or 145a | 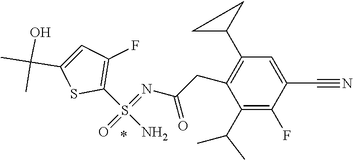 [SMILES: CC(C)c1c(F)c(C#N)cc(C2CC2)c1CC(=O)N=S(N)(=O)c1sc(C(C)(C)O)cc1F] | (S)- or (R)- 2-(4-cyano-6-cyclopropyl-3- fluoro-2-isopropylphenyl)-N-(3- fluoro-5-(2-hydroxypropan-2- yl)thiophen-2- ylsulfonimidoyl)acetamide | ChiralPak ID, 2*25 cm, 5 um | 30% IPA in Hex (0.1% FA) | 482.2 |
| 55 | 145a or 145b | 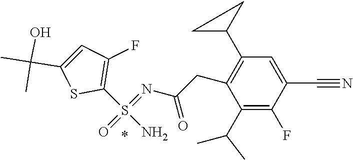 [SMILES: CC(C)c1c(F)c(C#N)cc(C2CC2)c1CC(=O)N=S(N)(=O)c1sc(C(C)(C)O)cc1F] | (R)- or (S)- 2-(4-cyano-6-cyclopropyl-3- fluoro-2-isopropylphenyl)-N-(3- fluoro-5-(2-hydroxypropan-2- yl)thiophen-2- ylsulfonimidoyl)acetamide | ChiralPak ID, 2*25 cm, 5 um | 30% IPA in Hex (0.1% FA) | 482.2 |
| 56 | 197b or 197a | 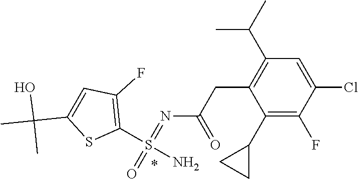 [SMILES: CC(C)c1cc(Cl)c(F)c(C2CC2)c1CC(=O)N=S(N)(=O)c1sc(C(C)(C)O)cc1F] | (S)- or (R)- 2-(4-chloro-2-cyclopropyl-3- fluoro-6-isopropylphenyl)-N-(3- fluoro-5-(2-hydroxypropan-2- yl)thiophen-2- ylsulfonimidoyl)acetamide | ChiralPak ID, 2*25 cm, 5 um | 30% IPA in Hex (0.1% DEA) | 491.2 |
| 57 | 197a or 197b | 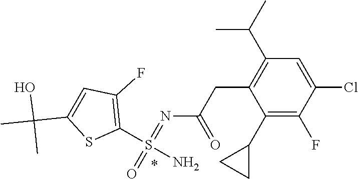 [SMILES: CC(C)c1cc(Cl)c(F)c(C2CC2)c1CC(=O)N=S(N)(=O)c1sc(C(C)(C)O)cc1F] | (R)- or (S)- 2-(4-chloro-2-cyclopropyl-3- fluoro-6-isopropylphenyl)-N-(3- fluoro-5-(2-hydroxypropan-2- yl)thiophen-2- ylsulfonimidoyl)acetamide | ChiralPak ID, 2*25 cm, 5 um | 30% IPA in Hex (0.1% DEA) | 491.2 |
| 58 | 116 | 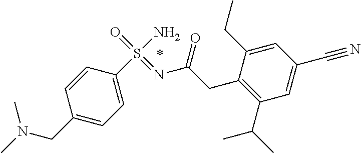 [SMILES: CCc1cc(C#N)cc(C(C)C)c1CC(=O)N=S(N)(=O)c1ccc(CN(C)C)cc1] | (S)- or (R)- 2-(4-cyano-2-ethyl-6- isopropylphenyl)-N-(4- ((dimethylamino)methyl)phenyl- sulfonimidoyl)acetamide | ChiralPak ADH, 2*25 cm, 5 um | 30% EtOH in Hex (0.1% DEA) | 427.2 |
| 59 | 116a or 116b | 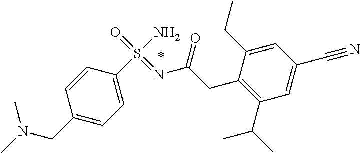 [SMILES: CCc1cc(C#N)cc(C(C)C)c1CC(=O)N=S(N)(=O)c1ccc(CN(C)C)cc1] | (R)- or (S)- 2-(4-cyano-2-ethyl-6- isopropylphenyl)-N-(4- ((dimethylamino)methyl)phenyl- sulfonimidoyl)acetamide | ChiralPak ADH, 2*25 cm, 5 um | 30% EtOH in Hex (0.1% DEA) | 427.2 |

| | | | | | | |
|---|---|---|---|---|---|---|
| 60 | 106 | 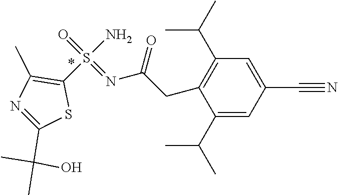 [SMILES: Cc1nc(C(C)(C)O)sc1S(N)(=O)=NC(=O)Cc1c(C(C)C)cc(C#N)cc1C(C)C] | (S)- or (R)- 2-(4-cyano-2,6- diisopropylphenyl)-N-(2-(2- hydroxypropan-2-yl)-4- methylthiazole-5- sulfonimidoyl)acetamide | ChiralPak IC, 2*25 cm, 5 um | 30% IPA in Hex (0.1% DEA) | 463.2 |
| 61 | 106a or 106b | 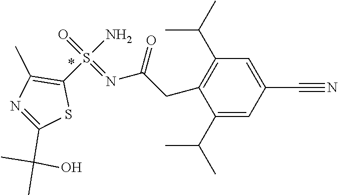 [SMILES: Cc1nc(C(C)(C)O)sc1S(N)(=O)=NC(=O)Cc1c(C(C)C)cc(C#N)cc1C(C)C] | (R)- or (S)- 2-(4-cyano-2,6- diisopropylphenyl)-N-(2-(2- hydroxypropan-2-yl)-4- methylthiazole-5- sulfonimidoyl)acetamide | ChiralPak IC, 2*25 cm, 5 um | 30% IPA in Hex (0.1% DEA) | 463.2 |
| 62 | 117a or 117b | 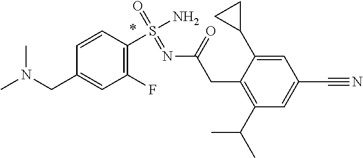 [SMILES: CC(C)c1cc(C#N)cc(C2CC2)c1CC(=O)N=S(N)(=O)c1ccc(CN(C)C)cc1F] | (S)- or (R)- 2-(4-cyano-2-cyclopropyl-6- isopropylphenyl)-N-(4- ((dimethylamino)methyl)-2- fluorophenylsulfonimidoyl) acetamide | ChiralCE L OD, 2*25 cm, 5 um | 30% EtOH in Hex (0.1% FA) | 457.2 |
| 63 | 117b or 117a | 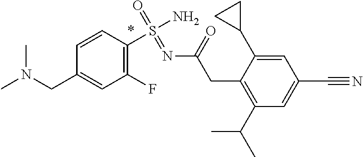 [SMILES: CC(C)c1cc(C#N)cc(C2CC2)c1CC(=O)N=S(N)(=O)c1ccc(CN(C)C)cc1F] | (R)- or (S)- 2-(4-cyano-2-cyclopropyl-6- isopropylphenyl)-N-(4- ((dimethylamino)methyl)-2- fluorophenylsulfonimidoyl) acetamide | ChiralCE L OD, 2*25 cm, 5 um | 30% EtOH in Hex (0.1% FA) | 457.2 |
| 64 | 152b or 152a | 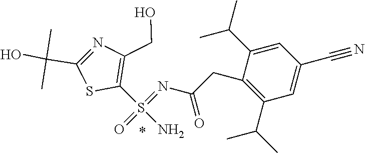 [SMILES: CC(C)c1cc(C#N)cc(C(C)C)c1CC(=O)N=S(N)(=O)c1sc(C(C)(C)O)nc1CO] | (S)- or (R)- 2-(4-cyano-2,6- diisopropylphenyl)-N-(4- (hydroxymethyl)-2-(2- hydroxypropan-2-yl)thiazol-5- ylsulfonimidoyl)acetamide | ChiralPak ID, 2*25 cm, 5 um | 80% EtOH in Hex (0.1% FA) | 480.2 |
| 65 | 152a or 152b | 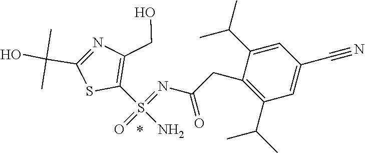 [SMILES: CC(C)c1cc(C#N)cc(C(C)C)c1CC(=O)N=S(N)(=O)c1sc(C(C)(C)O)nc1CO] | (R)- or (S)- 2-(4-cyano-2,6- diisopropylphenyl)-N-(4- (hydroxymethyl)-2-(2- hydroxypropan-2-yl)thiazol-5- ylsulfonimidoyl)acetamide | ChiralPak ID, 2*25 cm, 5 um | 80% EtOH in Hex (0.1% FA) | 480.2 |
| 66 | 150b or 150a | 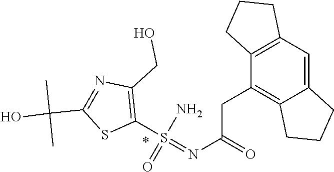 [SMILES: CC(C)(O)c1nc(CO)c(S(N)(=O)=NC(=O)Cc2c3c(cc4c2CCC4)CCC3)s1] | (S)- or (R)- 2-(1,2,3,5,6,7-hexahydros-indacen- 4-yl)-N-(4-(hydroxymethyl)-2-(2- hydroxypropan-2-yl)thiazol-5- ylsulfonimidoyl)acetamide | ChiralPak ID, 2*25 cm, 5 um | 88% IPA in Hex (0.1% FA) | 450.2 |
| 67 | 150a or 150b | 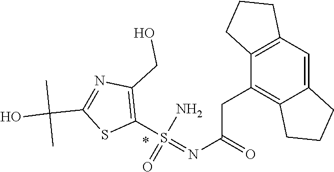 [SMILES: CC(C)(O)c1nc(CO)c(S(N)(=O)=NC(=O)Cc2c3c(cc4c2CCC4)CCC3)s1] | (R)- or (S)- 2-(1,2,3,5,6,7-hexahydros-indacen- 4-yl)-N-(4-(hydroxymethyl)-2-(2- hydroxypropan-2-yl)thiazol-5- ylsulfonimidoyl)acetamide | ChiralPak ID, 2*25 cm, 5 um | 88% IPA in Hex (0.1% FA) | 450.2 |
| 68 | 148b or 148a | 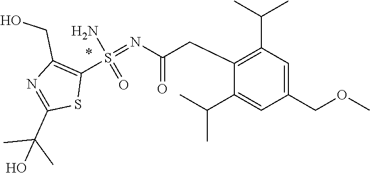 [SMILES: COCc1cc(C(C)C)c(CC(=O)N=S(N)(=O)c2sc(C(C)(C)O)nc2CO)c(C(C)C)c1] | (S)- or (R)- 2-(2,6-diisopropyl-4- (methoxymethyl)phenyl)-N-(4- (hydroxymethyl)-2-(2- hydroxypropan-2-yl)thiazol-5- ylsulfonimidoyl)acetamide | ChiralPak ID, 2*25 cm, 5 um | 75% IPA in Hex (0.1% FA) | 498.2 |
| 69 | 148a or 148b | 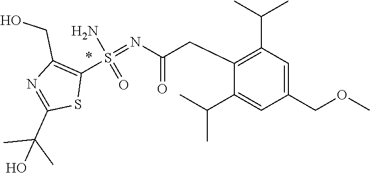 [SMILES: COCc1cc(C(C)C)c(CC(=O)N=S(N)(=O)c2sc(C(C)(C)O)nc2CO)c(C(C)C)c1] | (R)- or (S)- 2-(2,6-diisopropyl-4- (methoxymethyl)phenyl)-N-(4- (hydroxymethyl)-2-(2- hydroxypropan-2-yl)thiazol-5- ylsulfonimidoyl)acetamide | ChiralPak ID, 2*25 cm, 5 um | 75% IPA in Hex (0.1% FA) | 498.2 |
| 70 | 147b or 147a | 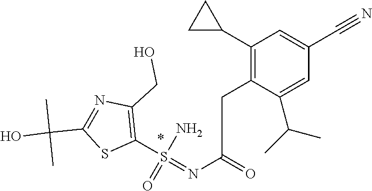 [SMILES: CC(C)c1cc(C#N)cc(C2CC2)c1CC(=O)N=S(N)(=O)c1sc(C(C)(C)O)nc1CO] | (S)- or (R)- 2-(4-cyano-2-cyclopropyl-6- isopropylphenyl)-N-(4- (hydroxymethyl)-2-(2- hydroxypropan-2-yl)thiazol-5- ylsulfonimidoyl)acetamide | ChiralPak IC, 2*25 cm, 5 um | 15% EtOH in Hex | 477.2 |
| 71 | 147a or 147b | 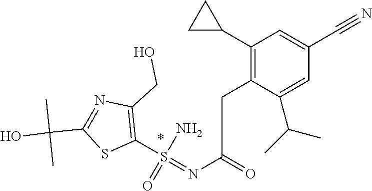 [SMILES: CC(C)c1cc(C#N)cc(C2CC2)c1CC(=O)N=S(N)(=O)c1sc(C(C)(C)O)nc1CO] | (R)- or (S)- 2-(4-cyano-2-cyclopropyl-6- isopropylphenyl)-N-(4- (hydroxymethyl)-2-(2- hydroxypropan-2-yl)thiazol-5- ylsulfonimidoyl)acetamide | ChiralPak IC, 2*25 cm, 5 um | 15% EtOH in Hex | 477.2 |
| 72 | 114b or 114a | 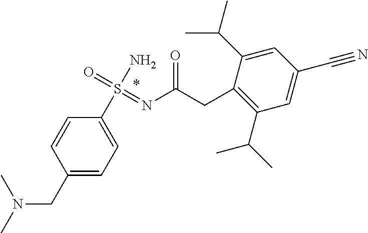 [SMILES: CC(C)c1cc(C#N)cc(C(C)C)c1CC(=O)N=S(N)(=O)c1ccc(CN(C)C)cc1] | (S)- or (R)- 2-(4-cyano-2,6- diisopropylphenyl)-N-(4- ((dimethylamino)methyl)phenyl- sulfonimidoyl)acetamide | ChiralPak ID, 2*25 cm, 5 um | 50% IPA in Hex (0.1% DEA) | 441.2 |
| 73 | 114a or 114b | 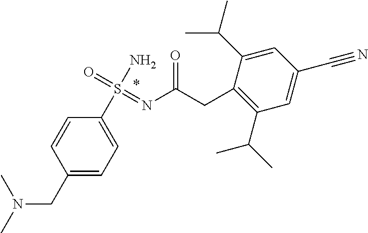 [SMILES: CC(C)c1cc(C#N)cc(C(C)C)c1CC(=O)N=S(N)(=O)c1ccc(CN(C)C)cc1] | (R)- or (S)- 2-(4-cyano-2,6- diisopropylphenyl)-N-(4- ((dimethylamino)methyl)phenyl- sulfonimidoyl)acetamide | ChiralPak ID, 2*25 cm, 5 um | 50% IPA in Hex (0.1% DEA) | 441.2 |
| 74 | 112b or 112a | 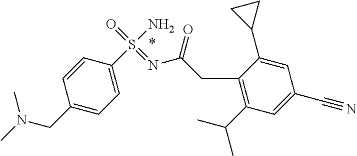 [SMILES: CC(C)c1cc(C#N)cc(C2CC2)c1CC(=O)N=S(N)(=O)c1ccc(CN(C)C)cc1] | (S)- or (R)- 2-(4-cyano-2-cyclopropyl-6- isopropylphenyl)-N-(4- ((dimethylamino)methyl)phenyl- sulfonimidoyl)acetamide | ChiralPak IG, 2*25 cm, 5 um | 25% IPA in Hex (0.1% DEA) | 439.2 |
| 75 | 112a or 112b | 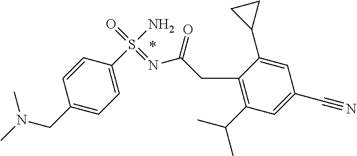 [SMILES: CC(C)c1cc(C#N)cc(C2CC2)c1CC(=O)N=S(N)(=O)c1ccc(CN(C)C)cc1] | (R)- or (S)- 2-(4-cyano-2-cyclopropyl-6- isopropylphenyl)-N-(4- ((dimethylamino)methyl)phenyl- sulfonimidoyl)acetamide | ChiralPak IG, 2*25 cm, 5 um | 25% EtOH in Hex (0.1% DEA) | 439.2 |
| 76 | 101b or 101a | 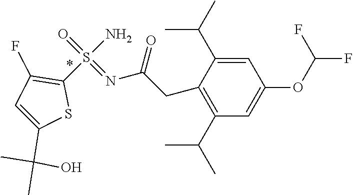 [SMILES: CC(C)c1cc(OC(F)F)cc(C(C)C)c1CC(=O)N=S(N)(=O)c1sc(C(C)(C)O)cc1F] | (S)- or (R)- 2-(4-(difluoromethoxy)-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% IPA in Hex (0.1% DEA) | 507.2 |
| 77 | 101a or 101b | 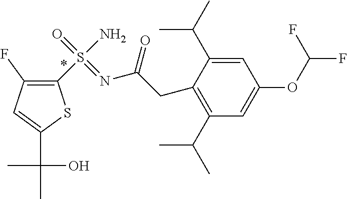 [SMILES: CC(C)c1cc(OC(F)F)cc(C(C)C)c1CC(=O)N=S(N)(=O)c1sc(C(C)(C)O)cc1F] | (R)- or (S)- 2-(4-(difluoromethoxy)-2,6- diisopropylphenyl)-N-(3-fluoro-5- (2-hydroxypropan-2-yl)thiophen- 2-ylsulfonimidoyl)acetamide | ChiralPak ID, 2*25 cm, 5 um | 30% IPA in Hex (0.1% DEA) | 507.2 |
| 78 | 126b or 126a | 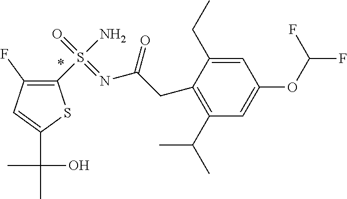 [SMILES: CCc1cc(OC(F)F)cc(C(C)C)c1CC(=O)N=S(N)(=O)c1sc(C(C)(C)O)cc1F] | (S)- or (R)- 2-(4-(difluoromethoxy)-2-ethyl-6- isopropylphenyl)-N-(3-fluoro-5-(2- hydroxypropan-2-yl)thiophen-2- ylsulfonimidoyl)acetamide | ChiralPak ID, 2*25 cm, 5 um | 25% IPA in Hex (0.1% DEA) | 493.2 |
| 79 | 126a or 126b | 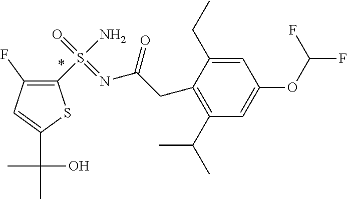 [SMILES: CCc1cc(OC(F)F)cc(C(C)C)c1CC(=O)N=S(N)(=O)c1sc(C(C)(C)O)cc1F] | (R)- or (S)- 2-(4-(difluoromethoxy)-2-ethyl-6- isopropylphenyl)-N-(3-fluoro-5-(2- hydroxypropan-2-yl)thiophen-2- ylsulfonimidoyl)acetamide | ChiralPak ID, 2*25 cm, 5 um | 25% IPA in Hex (0.1% DEA) | 493.2 |
| 80 | 104b or 104a | 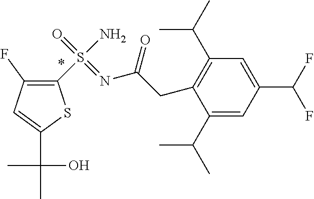 [SMILES: CC(C)c1cc(C(F)F)cc(C(C)C)c1CC(=O)N=S(N)(=O)c1sc(C(C)(C)O)cc1F] | (S)- or (R)- 2-(4-(difluoromethyl)-2,6- diisopropylphenyl)-N-(3-fluoro-5- (2-hydroxypropan-2-yl)thiophen- 2-ylsulfonimidoyl)acetamide | ChiralPak IC, 2*25 cm, 5 um | 15% IPA in Hex (0.1% FA) | 491.2 |
| 81 | 104a or 104b | 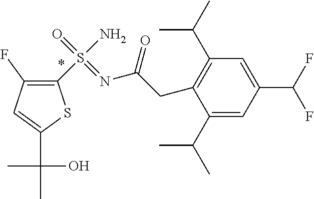 [SMILES: CC(C)c1cc(C(F)F)cc(C(C)C)c1CC(=O)N=S(N)(=O)c1sc(C(C)(C)O)cc1F] | (R)- or (S)- 2-(4-(difluoromethyl)-2,6- diisopropylphenyl)-N-(3-fluoro-5- (2-hydroxypropan-2-yl)thiophen- 2-ylsulfonimidoyl)acetamide | ChiralPak IC, 2*25 cm, 5 um | 15% IPA in Hex (0.1% FA) | 491.2 |
| 82 | 130b or 130a | 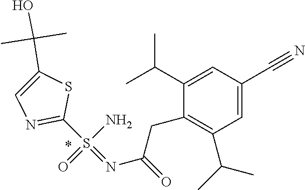 [SMILES: CC(C)c1cc(C#N)cc(C(C)C)c1CC(=O)N=S(N)(=O)c1ncc(C(C)(C)O)s1] | (R)- or (S)- 2-(4-cyano-2,6- diisopropylphenyl)-N-(5-(2- hydroxypropan-2-yl)thiazol-2- ylsulfonimidoyl)acetamide | ChiralPak IG, 2*25 cm, 5 um | 15% IPA in Hex (0.1% FA) | 449.2 |
| 83 | 130a or 130b | 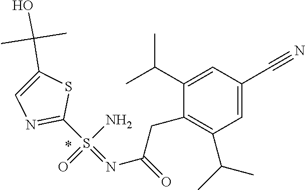 [SMILES: CC(C)c1cc(C#N)cc(C(C)C)c1CC(=O)N=S(N)(=O)c1ncc(C(C)(C)O)s1] | (S)- or (R)- 2-(4-cyano-2,6- diisopropylphenyl)-N-(5-(2- hydroxypropan-2-yl)thiazol-2- ylsulfonimidoyl)acetamide | ChiralPak IG, 2*25 cm, 5 um | 15% IPA in Hex (0.1% FA) | 449.2 |
| 84 | 107b or 107a | 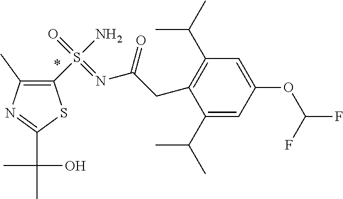 [SMILES: Cc1nc(C(C)(C)O)sc1S(N)(=O)=NC(=O)Cc1c(C(C)C)cc(OC(F)F)cc1C(C)C] | (S)- or (R)- 2-(4-(difluoromethoxy)-2,6- diisopropylphenyl)-N-(2-(2- hydroxypropan-2-yl)-4- methylthiazol-5- ylsulfonimidoyl)acetamide | ChiralPak IC, 2*25 cm, 5 um | 10% IPA in Hex (0.1% FA) | 504.2 |
| 85 | 107a or 107b | 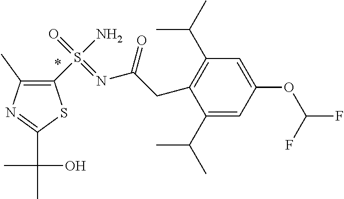 [SMILES: Cc1nc(C(C)(C)O)sc1S(N)(=O)=NC(=O)Cc1c(C(C)C)cc(OC(F)F)cc1C(C)C] | (R)- or (S)- 2-(4-(difluoromethoxy)-2,6- diisopropylphenyl)-N-(2-(2- hydroxypropan-2-yl)-4- methylthiazol-5- ylsulfonimidoyl)acetamide | ChiralPak IC, 2*25 cm, 5 um | 10% IPA in Hex (0.1% FA) | 504.2 |
| 86 | 109a or 109b | 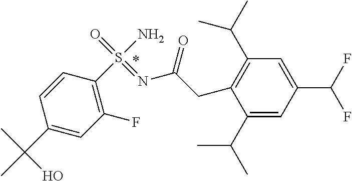 [SMILES: CC(C)c1cc(C(F)F)cc(C(C)C)c1CC(=O)N=S(N)(=O)c1ccc(C(C)(C)O)cc1F] | (S)- or (R)- 2-(4-(difluoromethyl)-2,6- diisopropylphenyl)-N-(2-fluoro-4- (2-hydroxypropan-2- yl)phenylsulfonimidoyl)acetamide | ChiralPak IF, 2*25 cm, 5 um | 15% EtOH in Hex (0.1% DEA) | 485.2 |
| 87 | 109b or 109a | 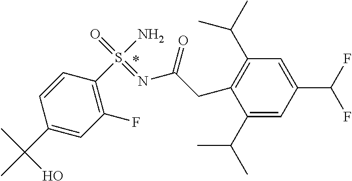 [SMILES: CC(C)c1cc(C(F)F)cc(C(C)C)c1CC(=O)N=S(N)(=O)c1ccc(C(C)(C)O)cc1F] | (R)- or (S)- 2-(4-(difluoromethyl)-2,6- diisopropylphenyl)-N-(2-fluoro-4- (2-hydroxypropan-2- yl)phenylsulfonimidoyl)acetamide | ChiralPak IF, 2*25 cm, 5 um | 15% EtOH in Hex (0.1% DEA) | 485.2 |
| 88 | 201b | 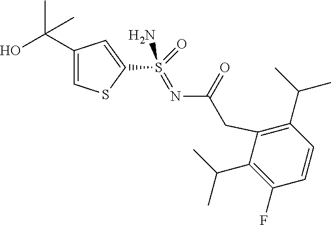 [SMILES: CC(C)c1ccc(F)c(C(C)C)c1CC(=O)N=[S@@](N)(=O)c1cc(C(C)(C)O)cs1] | (R)-N-(amino(4-(2- hydroxypropan-2-yl)thiophen-2- yl)oxo)-λ⁶-sulfanylidene)-2-(3- fluoro-2,6- diisopropylphenyl)acetamide | ChiralPak IF, 2*25 cm, 5 um | 15% EtOH in Hex (0.1% DEA) | 440.59 |
| 89 | 301 | 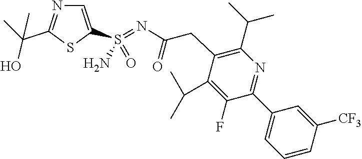 [SMILES: CC(C)c1nc(-c2cccc(C(F)(F)F)c2)c(F)c(C(C)C)c1CC(=O)N=[S@@](N)(=O)c1cnc(C(C)(C)O)s1] | (R)-N-(amino(2-(2- hydroxypropan-2-yl)thiazol-5- yl)oxo)-λ⁶-sulfanylidene)-2-(5- fluoro-2,4-diisopropyl-6-(3- (trifluoromethyl)phenyl)pyridin-3- yl)acetamide | ChiralPak IF, 2*25 cm, 5 um | 15% EtOH in Hex (0.1% DEA) | 586.66 |

### Example 88 (Compound 241)

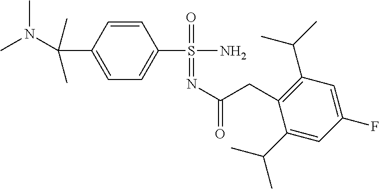 [SMILES: CC(C)c1cc(F)cc(C(C)C)c1CC(=O)N=S(N)(=O)c1ccc(C(C)(C)N(C)C)cc1]

### N-(amino(4-(2-(dimethylamino)propan-2-yl)phenyl)(oxo)-)λ⁶-sulfaneylidene)-2-(4-fluoro-2,6-diisopropylphenyl)acetamide (Scheme 1A)

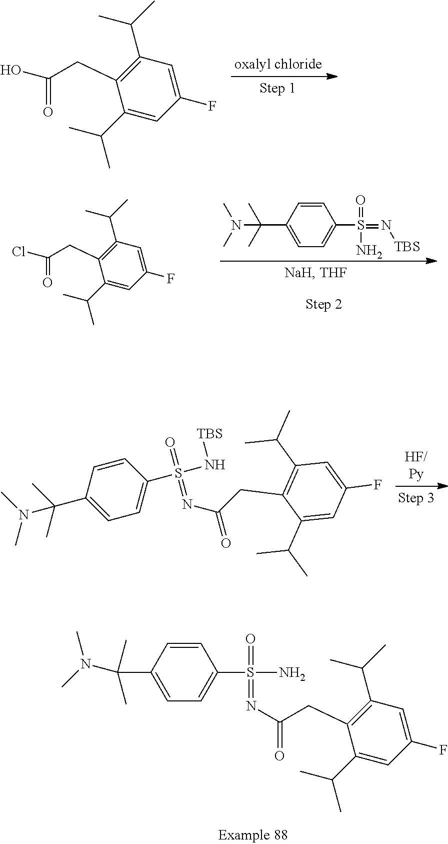

### Step 1: 2-(4-Fluoro-2,6-diisopropylphenyl)acetyl chloride

Into a 25-mL round-bottom flask, was placed a solution of 2-[4-fluoro-2,6-bis(propan-2-yl)phenyl] acetic acid (20 mg, 0.08 mmol) in DCM (2 mL). This was followed by the addition of DMF (0.005 mL) with stirring. To this was added oxalic dichloride (0.5 mL) dropwise with stirring at 0° C. The resulting solution was stirred for 1 h at RT. The resulting mixture was concentrated. This resulted in 21 mg (97.4%) of the title compound as a yellow solid. This crude product was used in the next step.

### Step 2: N-(((tert-butyldimethylsilyl)amino)(4-(2-(dimethylamino)propan-2-yl)phenyl)(oxo)-λ⁶-sulfaneylidene)-2-(4-fluoro-2,6-diisopropylphenyl)acetamide

Into a 25-mL round-bottom flask, was placed a solution of N-(tert-butyldimethylsilyl)-4-[2-(dimethylamino) propan-2-yl]benzene-1-sulfonoimidamide (20 mg, 0.06 mmol) in THF (3 mL). To this was added NaH (60% wt. oil dispersion, 12 mg, 0.3 mmol) at 0° C. To the mixture was added a solution of 2-[4-fluoro-2,6-bis(propan-2-yl) phenyl]acetyl chloride (14.4 mg, 0.06 mmol) in DCM (1 mL) dropwise with stirring at 0° C. The resulting solution was stirred for 1 h at RT. The reaction was then quenched by the addition of 20 mL of water. The resulting solution was extracted with 2×25 mL of ethyl acetate, dried over anhydrous sodium sulfate, and concentrated. The residue was eluted from silica gel with ethyl acetate/petroleum ether (1:1). This resulted in 20 mg (61.7%) of the title compound as a white solid. MS-ESI: 576 (M+1).

### Step 3: N-(amino(4-(2-(dimethylamino)propan-2-yl)phenyl)(oxo)-λ⁶-sulfaneylidene)-2-(4-fluoro-2,6-diisopropylphenyl)acetamide

Into a 25-mL round-bottom flask, was placed a solution of N-[[(tert-butyldimethylsilyl)amino]([4-[2-(dimethylamino)propan-2-yl]phenyl])oxo-λ⁶-sulfanylidene]-2-[4-fluoro-2,6-bis(propan-2-yl)phenyl]acetamide (20 mg, 0.03 mmol) in DCM (2 mL). This was followed by the addition of HF-Pyridine (70% wt., 1 mL) dropwise with stirring. The resulting solution was stirred for 30 min at RT. The resulting mixture was washed with 20 mL of H₂O. The resulting solution was extracted with 2×25 mL of ethyl acetate dried over anhydrous sodium sulfate. The resulting mixture was concentrated. The crude product was purified by Prep-HPLC with the following conditions: Column, XBridge Shield RP18 OBD, 19*250 mm, 10 um; mobile phase, water (10 mM NH₄HCO₃) and ACN (30% to 50% ACN gradient in 10 min); Detector, UV220/254 nm. This resulted in 5.2 mg (32.4%) of Example 88 as a white solid. MS-ESI: 462.3 (M+1). ¹H-NMR (400 MHz, CD₃OD-d₄) δ 7.87 (d, 0.1=8.4 Hz, 2H), 7.69 (d, J=8.4 Hz, 2H), 6.76 (d, J=10.4 Hz, 2H), 3.74 (s, 2H), 3.12-3.08 (m, 2H), 2.16 (s, 6H), 1.39 (s, 6H), 1.11 (dd, J=15.2, 6.8 Hz, 12H).

TABLE 18
Examples in the following table were prepared using similar conditions as described in Example 88 and Scheme 1A from
appropriate starting materials.

| Example | Compound | | | Exact Mass |
|---|---|---|---|---|
| # | # | Structure | IUPAC Name | [M + H]⁺ |
| 89 | 219 | 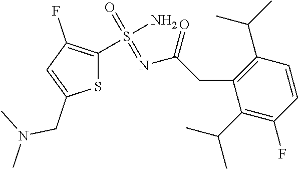 [SMILES: CC(C)c1ccc(F)c(C(C)C)c1CC(=O)N=S(N)(=O)c1sc(CN(C)C)cc1F] | N-(amino(5- ((dimethylamino)methyl)-3- fluorothiophen-2-yl)(oxo)-λ⁶- sulfaneylidene)-2-(3-fluoro-2,6- diisopropylphenyl)acetamide | 458 |
| 90 | 218 | 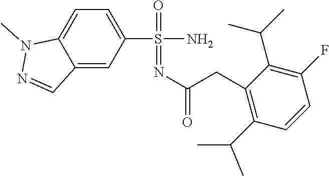 [SMILES: CC(C)c1ccc(F)c(C(C)C)c1CC(=O)N=S(N)(=O)c1ccc2c(cnn2C)c1] | N-(amino(1-methyl-1H- indazol-5-yl)(oxo)-λ⁶- sulfaneylidene)-2-(3-fluoro-2,6- diisopropylphenyl)acetamide | 431 |
| 91 | 217 | 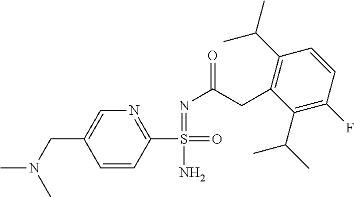 [SMILES: CC(C)c1ccc(F)c(C(C)C)c1CC(=O)N=S(N)(=O)c1ccc(CN(C)C)cn1] | N-(amino(5- ((dimethylamino)methyl) pyridin-2-yl)(oxo)-λ⁶- sulfaneylidene)-2-(3-fluoro-2,6- diisopropylphenyl)acetamide | 435 |
| 92 | 214 | 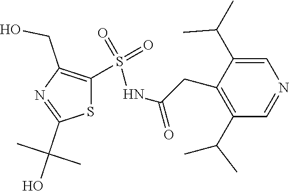 [SMILES: CC(C)c1cncc(C(C)C)c1CC(=O)NS(=O)(=O)c1sc(C(C)(C)O)nc1CO] | 2-(3,5-diisopropylpyridin-4- yl)-N-((4-(hydroxymethyl)-2- (2-hydroxypropan-2-yl)thiazol- 5-yl)sulfonyl)acetamide | 456 |

### Example 93 (Compound 235)

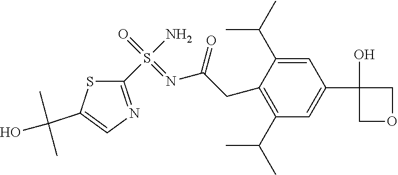 [SMILES: CC(C)c1cc(C2(O)COC2)cc(C(C)C)c1CC(=O)N=S(N)(=O)c1ncc(C(C)(C)O)s1]

### N-(amino(5-(2-hydroxypropan-2-yl)thiazol-2-yl)(oxo)-λ⁶-sulfaneylidene)-2-(4-(3-hydroxyoxetan-3-yl)-2,6-diisopropylphenyl)acetamide (Scheme 1B)

### Example 94 (Compound 230)

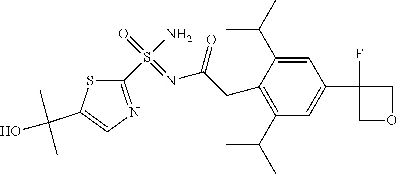 [SMILES: CC(C)c1cc(C2(F)COC2)cc(C(C)C)c1CC(=O)N=S(N)(=O)c1ncc(C(C)(C)O)s1]

### N-(amino(5-(2-hydroxypropan-2-yl)thiazol-2-yl)(oxo)-λ⁶-sulfaneylidene)-2-(4-(3-fluorooxetan-3-yl)-2,6-diisopropylphenyl)acetamide (Scheme 1B)

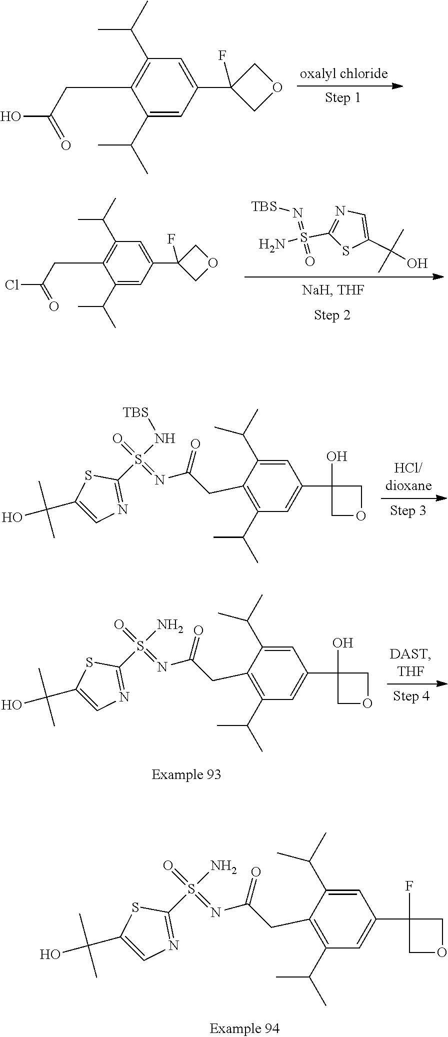

### Step 1: 2-(4-(3-fluorooxetan-3-yl)-2,6-diisopropylphenyl)acetyl chloride

Into a 50-mL round-bottom flask, was placed 2-[4-(3-fluorooxetan-3-yl)-2,6-bis(propan-2-yl)phenyl] acetic acid (150 mg, 0.51 mmol) in DCM (3 mL) and DMF (0.05 mL). This was followed by the addition of oxalic dichloride (0.5 mL) dropwise with stirring at RT. The resulting solution was stirred for 30 min at RT. The resulting mixture was concentrated under vacuum. This resulted in 159 mg (99.7%) of the title compound as a light yellow solid.

### Step 2: N-(((tert-butyldimethylsilyl)amino)(5-(2-hydroxypropan-2-yl)thiazol-2-yl)(oxo)-λ⁶-sulfaneylidene)-2-(4-(3-hydroxyoxetan-3-yl)-2,6-diisopropylphenyl)acetamide

Into a 50-mL round-bottom flask, was placed N-(tert-butyldimethylsilyl)-5-(2-hydroxypropan-2-yl)-1,3-thiazole-2-sulfonoimidamide (173 mg, 0.52 mmol) in THF (5 mL). This was followed by the addition of NaH (60% wt. oil dispersion, 62.4 mg, 1.56 mmol) in portions at 0° C. The resulting solution was stirred for 10 min at RT. Then to the above was added a solution of 2-[4-(3-fluorooxetan-3-yl)-2,6-bis(propan-2-yl)phenyl]acetyl chloride (159 mg, 0.51 mmol) in THF (2 mL) dropwise with stirring at 0° C. The resulting solution was stirred for 2 h at RT. The reaction was then quenched by the addition of 5 mL of water. The resulting solution was extracted with 3×5 mL of ethyl acetate. The organic layers were combined, dried over anhydrous sodium sulfate, and concentrated under vacuum. This resulted in 200 mg (64.5%) of the title compound as an off-white solid. MS-ESI: 610 (M+1).

### Step 3: N-(amino(5-(2-hydroxypropan-2-yl)thiazol-2-yl)(oxo)-1′-sulfaneylidene)-2-(4-(3-hydroxyoxetan-3-yl)-2,6-diisopropylphenyl)acetamide

Into a 50-mL round-bottom flask, was placed N-[[(tert-butyldimethylsilyl)amino][5-(2-hydroxypropan-2-yl)-1,3-thiazol-2-yl]oxo-)⁶-sulfanylidene]-2-[4-(3-hydroxyoxetan-3-yl)-2,6-bis(propan-2-yl)phenyl]acetamide (200 mg, 0.33 mmol) in THF (2 mL), to the stirred solution was added HCl/dioxane (4 M, 5 mL). The resulting solution was stirred overnight at RT. The resulting mixture was concentrated under vacuum. The crude product was purified by Prep-HPLC with the following conditions: Column, XBridge Prep C18 OBD, 5 μm, 19*150 mm; mobile phase, water (10 mM NH₄HCO₃) and ACN (5% to 40% ACN gradient over 8 min); Detector, UV 254/210 nm. This resulted in 14.0 mg (8.61%) of Example 93 as a white solid. MS-ESI: 496.3 (M+1). ¹H-NMR: (DMSO-d₆, 400 MHz): δ 7.40 (s, 1H), 7.23 (s, 2H), 6.14 (s, 1H), 5.54 (s, 11H), 4.73-4.66 (m, 4H), 3.92 (s, 1H), 3.49-3.46 (m, 2H), 3.31-3.22 (m, 2H), 1.47 (s, 6H), 1.12 (d, J=8.4 Hz, 6H), 1.10 (d, J=8.4 Hz, 6H).

### Step 4: N-(amino(5-(2-hydroxypropan-2-yl)thiazol-2-yl)(oxo)-λ⁶-sulfaneylidene)-2-(4-(3-fluorooxetan-3-yl)-2,6-diisopropylphenyl)acetamide

Into a 50-mL 3-necked round-bottom flask purged and maintained with an inert atmosphere of nitrogen, was placed N-[amino[5-(2-hydroxypropan-2-yl)-1,3-thiazol-2-yl]oxo-λ⁶-sulfanylidene]-2-[4-(3-hydroxyoxetan-3-yl)-2,6-bis(propan-2-yl)phenyl]acetamide (83 mg, 0.17 mmol) in THF (5 mL). This was followed by the addition of a solution of DAST (54.0 mg, 0.33 mmol) in DCM (1 mL) dropwise with stirring at 0° C. The resulting solution was stirred overnight at RT. The resulting mixture was concentrated under vacuum. The crude product was purified by Prep-HPLC with the following conditions: Column, XBridge BEH130 Prep C18 OBD, 19×150 mm, 5 μm 13 nm; mobile phase, water (10 mM NH₄HCO₃) and ACN (30% to 60% ACN gradient in 7 min); Detector, UV 254/210 nm. This resulted in 15.1 mg (17.9%) of Example 94 as a white solid. MS-ESI: 498.2 (M+1). ¹H NMR (DMSO-d₆, 400 MHz): δ 7.96 (s, 2H), 7.76 (s, 1H), 7.18 (s, 2H), 5.87 (s, 1H), 4.98-4.87 (m, 4H), 3.77-3.65 (m, 2H), 3.13-3.05 (m, 2H), 1.51 (s, 6H), 1.13 (d, J=8.4 Hz, 6H), 1.11 (d, J=8.4 Hz, 6H).

TABLE 19
Examples in the following table were prepared using similar conditions as described in Example 94 and
Scheme 1B from appropriate materials

| | | | | Exact |
|---|---|---|---|---|
| Example | Compound | | | Mass |
| # | # | Structure | IUPAC Name | [M + H]⁺ |
| 95 | 245 | 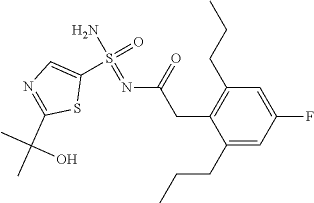 [SMILES: CCCc1cc(F)cc(CCC)c1CC(=O)N=S(N)(=O)c1cnc(C(C)(C)O)s1] | N-(amino(2-(2-hydroxypropan- 2-yl)thiazol-5-yl)(oxo)-λ⁶- sulfaneylidene)-2-(4-fluoro-2,6- dipropylphenyl)acetamide | 442 |

### Example 96 (Compound 244)

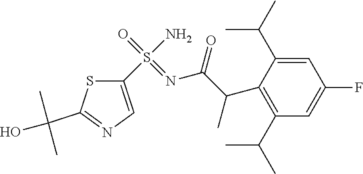 [SMILES: CC(C)c1cc(F)cc(C(C)C)c1C(C)C(=O)N=S(N)(=O)c1cnc(C(C)(C)O)s1]

### N-(amino(2-(2-hydroxypropan-2-yl)thiazol-5-yl)(oxo)-λ⁶-sulfaneylidene)-2-(4-fluoro-2,6-diisopropylphenyl)propanamide (Scheme 1C)

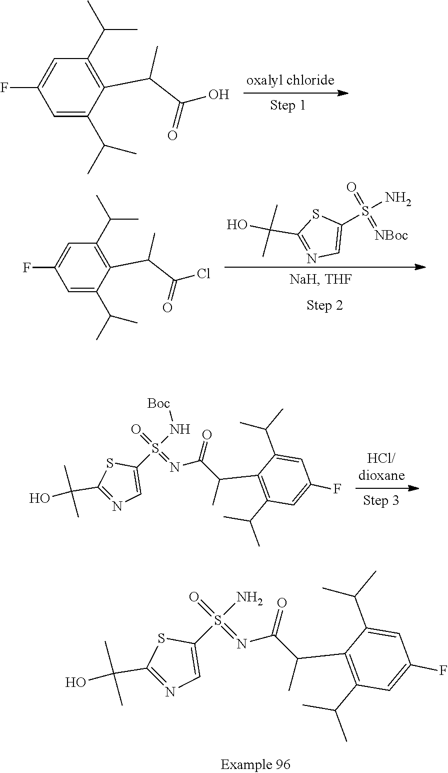

### Step 1: 2-(4-Fluoro-2,6-diisopropylphenyl)propanoyl chloride

Into a 50-mL round-bottom flask, was placed 2-[4-fluoro-2,6-bis(propan-2-yl)phenyl]propanoic acid (200 mg, 0.79 mmol) in DCM (20 mL), this was followed by the addition of oxalic dichloride (2 mL). The resulting solution was stirred for 15 min at RT. The resulting mixture was concentrated. This resulted in 200 mg (93.1%) of the title compound as off-white oil.

### Step 2: Tert-butyl(N-(2-(4-fluoro-2,6-diisopropylphenyl)propanoyl)-2-(2-hydroxypropan-2-yl) thiazole-5-sulfonimidoyl)carbamate

Into a 50-mL round-bottom flask, was placed tert-butyl N-[amino[2-(2-hydroxypropan-2-yl)-1,3-thiazol-5-yl] methylidene-λ⁶-sulfanylidene]carbamate (235.9 mg, 0.74 mmol) in THF (20 mL). To the mixture was added 2-[4-fluoro-2,6-bis(propan-2-yl)phenyl]propanoyl chloride (200 mg, 0.74 mmol) and NaH (60% wt. oil dispersion, 59.2 mg, 1.48 mmol). The resulting solution was stirred for 16 h at RT. The reaction was then quenched by the addition of 20 mL of water. The resulting solution was extracted with 3×20 ml of ethyl acetate, dried over anhydrous sodium sulfate, and concentrated. The resulting mixture was concentrated. The residue was eluted from silica gel with ethyl acetate/petroleum ether (1:1). This resulted in 200 mg (48.9%) of the title compound as an off-white solid. MS-ESI: 556 (M+1).

### Step 3: N-(amino(2-(2-hydroxypropan-2-yl)thiazol-5-yl)(oxo)-λ⁶-sulfaneylidene)-2-(4-fluoro-2,6-diisopropylphenyl)propanamide

Into a 50-mL round-bottom flask, was placed tert-butyl N-[([2-[4-fluoro-2,6-bis(propan-2-yl)phenyl]propanoyl]imino)[2-(2-hydroxypropan-2-yl)-1,3-thiazol-5-yl]methylidene-λ⁶-sulfanyl]carbamate (100 mg, 0.18 mmol) in THF (5 mL). To the stirred solution was added HCl/dioxane (10 mL, 4 M) dropwise. The resulting solution was stirred for 16 h at RT. The resulting mixture was concentrated. The crude product was purified by Prep-HPLC with the following conditions: Column: XBridge Shield RP18 OBD Column 19*250 mm, 10 um; Mobile Phase A: water (10 mM NH₄HCO₃), Mobile Phase B: ACN; Flow rate: 25 mL/min; Gradient: 27% B to 45% B in 9m; 254/210 nm; Rt: 7.77 m. This resulted in 20 mg (24.4%) of Example 96 as a white solid. MS-ESI: 456.2 (M+1). ¹H-NMR (400 MHz, DMSO-d₆, ppm) δ: 8.00-7.92 (m, 3H), 7.05-6.91 (m, 2H), 6.25 (s, 11H), 3.87-3.81 (m, 11H), 3.16-2.98 (m, 2H), 1.49 (s, 6H), 1.27-1.24 (m, 3H), 1.19-1.13 (m, 12H).

TABLE 20
Examples in the following table were prepared using similar conditions as described in Example 96 and Scheme 1C from appropriate
starting materials.

| | | | | Exact |
|---|---|---|---|---|
| Example | Compound | | | Mass |
| # | # | Structure | IUPAC Name | [M + H]⁺ |
| 97 | 221 | 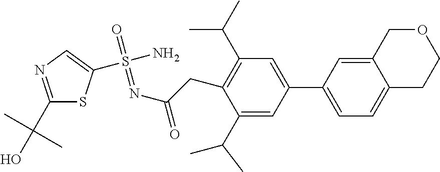 [SMILES: CC(C)c1cc(-c2ccc3c(c2)COCC3)cc(C(C)C)c1CC(=O)N=S(N)(=O)c1cnc(C(C)(C)O)s1] | N-(amino(2-(2- hydroxypropan-2-yl)thiazol- 5-yl)(oxo)-λ⁶- sulfaneylidene)-2-(4- (isochroman-7-yl)-2,6- diisopropylphenyl)acetamide | 556 |
| 98 | 220 | 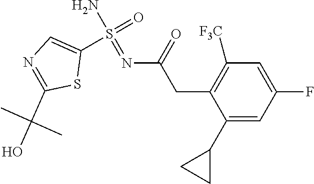 [SMILES: CC(C)(O)c1ncc(S(N)(=O)=NC(=O)Cc2c(C3CC3)cc(F)cc2C(F)(F)F)s1] | N-(amino(2-(2- hydroxypropan-2-yl)thiazol- 5-yl(oxo)-λ⁶- sulfaneylidene)-2-(2- cyclopropyl-4-fluoro-6- (trifluoromethyl)phenyl) acetamide | 466 |
| 99 | 304 | 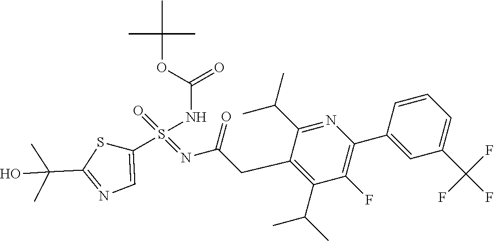 [SMILES: CC(C)c1nc(-c2cccc(C(F)(F)F)c2)c(F)c(C(C)C)c1CC(=O)N=S(=O)(NC(=O)OC(C)(C)C)c1cnc(C(C)(C)O)s1] | tert-butyl (N-(2-(5-fluoro- 2,4-diisopropyl-6-(3- (trifluoromethyl)phenyl) pyridin-3-yl)acetyl)-2-(2- hydroxypropan-2- yl)thiazole-5- sulfonimidoyl)carbamate | 687 |
| 100 | 301 | 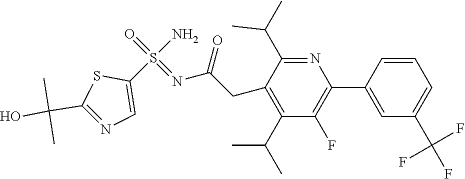 [SMILES: CC(C)c1nc(-c2cccc(C(F)(F)F)c2)c(F)c(C(C)C)c1CC(=O)N=S(N)(=O)c1cnc(C(C)(C)O)s1] | N-(amino(2-(2- hydroxypropan-2-yl)thiazol- 5-yl(oxo)-λ⁶- sulfaneylidene)-2-(5-fluoro- 2,4-diisopropyl-6-(3- (trifluoromethyl)phenyl) pyridin-3-yl)acetamide | 587 |
| 101 | 212 | 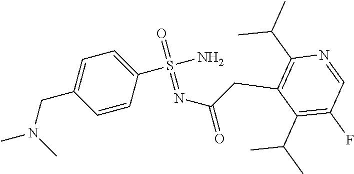 [SMILES: CC(C)c1ncc(F)c(C(C)C)c1CC(=O)N=S(N)(=O)c1ccc(CN(C)C)cc1] | N-(amino(4- ((dimethylamino)methyl) phenyl)(oxo)-λ⁶-sulfaneylidene)- 2-(5-fluoro-2,4- diisopropylpyridin-3- yl)acetamide | 435 |

### Example 102 (Compound 240)

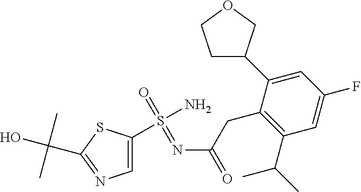 [SMILES: CC(C)c1cc(F)cc(C2CCOC2)c1CC(=O)N=S(N)(=O)c1cnc(C(C)(C)O)s1]

### N-(amino(2-(2-hydroxypropan-2-yl)thiazol-5-yl)(oxo)-λ⁶-sulfaneylidene)-2-(4-fluoro-2-isopropyl-6-(tetrahydrofuran-3-yl)phenyl)acetamide (Scheme 1C)

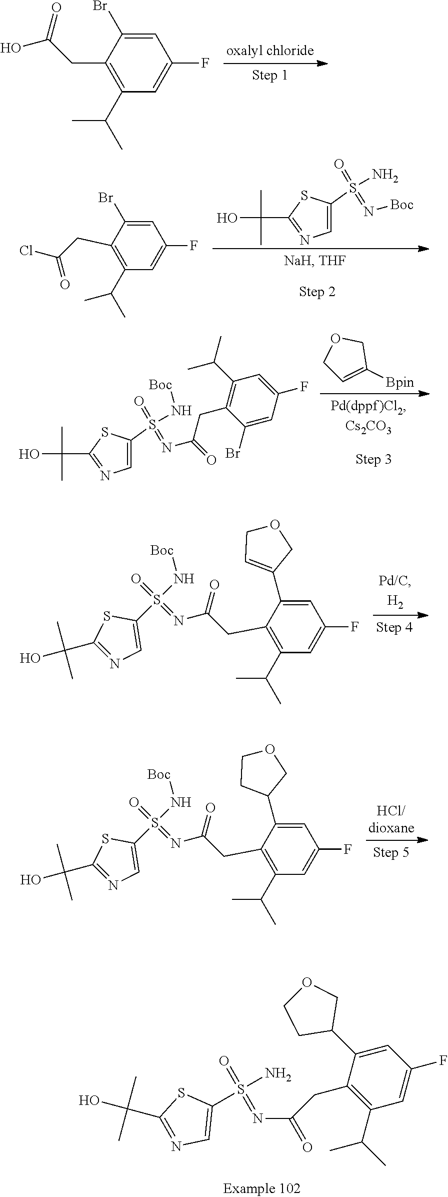

### Step 1: 2-(2-Bromo-4-fluoro-6-isopropylphenyl)acetyl chloride

Into a 100-mL round-bottom flask, was placed 2-[2-bromo-4-fluoro-6-(propan-2-yl)phenyl]acetic acid (100 mg, 0.36 mmol) in DCM (25 mL) and DMF (0.01 mL). To the above solution was added oxalic dichloride (0.5 mL) dropwise. The resulting solution was stirred for 30 min at RT. The resulting mixture was concentrated. This resulted in 100 mg (93.7%) of the title compound as yellow oil.

### Step 2: Tert-butyl (N-(2-(2-bromo-4-fluoro-6-isopropylphenyl)acetyl)-2-(2-hydroxypropan-2-yl) thiazole-5-sulfonimidoyl)carbamate

Into a 100-mL round-bottom flask, was placed tert-butyl N-[amino[2-(2-hydroxypropan-2-yl)-1,3-thiazol-5-yl]oxo-λ⁶-sulfanylidene]carbamate (116.7 mg, 0.36 mmol) in THF (25 mL). To the mixture was added NaH (60% wt. oil dispersion, 29.2 mg, 0.73 mmol) in portions with stirring. The resulting solution was stirred for 30 min at RT. 2-[2-bromo-4-fluoro-6-(propan-2-yl)phenyl]acetyl chloride (100 mg, 0.34 mmol) was added to the solution. The resulting solution was stirred for an additional 1 h at RT. The reaction was then quenched by the addition of 5 mL of water. The resulting solution was extracted with 2×10 ml of ethyl acetate and concentrated. This resulted in 219 mg (crude) title compound as a white solid. MS-ESI: 578 (M+1).

### Step 3: Tert-butyl (N-(2-(2-(2, 5-dihydrofuran-3-yl)-4-fluoro-6-isopropylphenyl)acetyl)-2-(2-hydroxypropan-2-yl)thiazole-5-sulfonimidoyl)carbamate

Into a 100-mL round-bottom flask purged and maintained with an inert atmosphere of nitrogen, was placed tert-butyl (N-(2-(2-bromo-4-fluoro-6-isopropylphenyl)acetyl)-2-(2-hydroxypropan-2-yl) thiazole-5-sulfonimidoyl)carbamate (395 mg, 0.68 mmol) in dioxane (60 mL) and H₂O (12 mL). This was followed by the addition of Cs₂CO₃ (667.4 mg, 2.05 mmol), 2-(2, 5-dihydrofuran-3-yl)-4,4, 5, 5-tetramethyl-1,3,2-dioxaborolane (201 mg, 1.02 mmol) and Pd(dppf)Cl₂ (50 mg, 0.07 mmol). The resulting solution was stirred for 16 h at 95° C. in an oil bath. The resulting solution was diluted with H₂O (50 mL), extracted with 2×15 ml of ethyl acetate and concentrated. The residue was eluted from silica gel with ethyl acetate/petroleum ether (1:1). This resulted in 187 mg (50.9%) of the title compound as a dark orange solid. MS-ESI: 568 (M+1).

### Step 4: Tert-butyl (N-(2-(4-fluoro-2-isopropyl-6-(tetrahydrofuran-3-yl)phenyl)acetyl)-2-(2-hydroxypropan-2-yl)thiazole-5-sulfonimidoyl)carbamate

Into a 100-mL round-bottom flask, was placed tert-butyl N-([2-[2-(2, 5-dihydrofuran-3-yl)-4-fluoro-6-(propan-2-yl)phenyl]acetamido][2-(2-hydroxypropan-2-yl)-1,3-oxazol-5-yl]oxo-λ⁶-sulfanylidene)carbamate (237 mg, 0.43 mmol) in methanol (25 mL). To the above solution was added Pd/C (10% wt., 30 mg) with stirring. The solution was evacuated and filled three times with hydrogen. The resulting solution was stirred for 16 h at RT. The solids were filtered out. The resulting mixture was concentrated. The residue was eluted from silica gel with DCM/methanol (10:1). This resulted in 234 mg (98.3%) of the title compound as a white solid. MS-ESI: 570 (M+1).

### Step 5: N-(amino(2-(2-hydroxypropan-2-yl)thiazol-5-yl)(oxo)-λ⁶-sulfaneylidene)-2-(4-fluoro-2-isopropyl-6-(tetrahydrofuran-3-yl)phenyl)acetamide

Into a 50-mL round-bottom flask, was placed tert-butyl N-[([2-[4-fluoro-2-(oxolan-3-yl)-6-(propan-2-yl)phenyl]acetyl]imino)[2-(2-hydroxypropan-2-yl)-1,3-thiazol-5-yl]oxo-λ⁶-sulfanyl]carbamate (200 mg, 0.35 mmol) in HCl/dioxane (4M, 10 mL). The resulting solution was stirred for 1 h at RT. The resulting mixture was concentrated. The crude product was purified by Prep-HPLC with the following conditions: Column, XBridge Prep C18 OBD, 5 μm, 19*150 mm; mobile phase, water (10 mM NH₄HCO₃) and ACN (7% to 45% ACN gradient in 7 min); Detector, UV 254/210 nm. This resulted in 20 mg (12.13%) of Example 102 as a white solid. MS-ESI: 470 (M+1). ¹H-NMR (400 MHz, DMSO-d₆, ppm) δ: 8.06 (br s, 2H), 7.03 (d, J=8.0 Hz, 1H), 7.03 (d, J=12.4 Hz, 1H), 6.28 (s, 1H), 4.01-3.97 (m, 1H), 3.91-3.89 (m, 1H), 3.79-3.77 (m, 1H), 3.55 (s, 2 H), 3.55-3.45 (m, 3H), 2.91-2.86 (m, 1H), 2.25-2.23 (m, 1H), 1.48 (s, 6H), 1.06 (d, J=6.8 Hz, 3H), 1.01 (d, J=6.8 Hz, 3H).

TABLE 21
Examples in the following table were prepared using similar conditions as described in Example 4 and Scheme 2 from appropriate starting materials.

| Exam- | Com- | | | Exact |
|---|---|---|---|---|
| ple | pound | | | Mass |
| # | # | Structure | IUPAC Name | [M + H]⁺ |
| 103 | 258 | 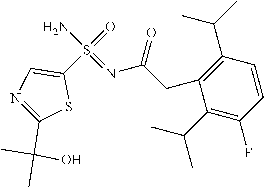 [SMILES: CC(C)c1ccc(F)c(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(3-fluoro- 2,6- diisopropylphenyl)acetamide | 442 |
| 104 | 257 | 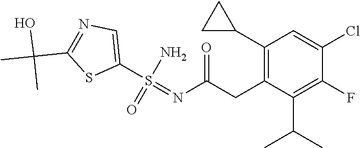 [SMILES: CC(C)c1c(F)c(Cl)cc(C2CC2)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4- chloro-6-cyclopropyl-3- fluoro-2- isopropylphenyl)acetamide | 474 |
| 105 | 256 | 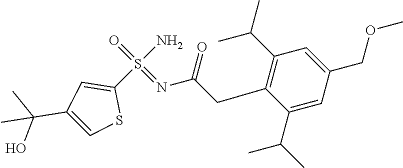 [SMILES: COCc1cc(C(C)C)c(CC(=O)N=S(N)(=O)c2cc(C(C)(C)O)cs2)c(C(C)C)c1] | N-(amino(4-(2- hydroxypropan-2- yl)thiophen-2-yl)(oxo)-λ⁶- sulfaneylidene)-2-(2,6- diisopropyl-4- (methoxymethyl)phenyl) acetamide | 467 |
| 106 | 201 | 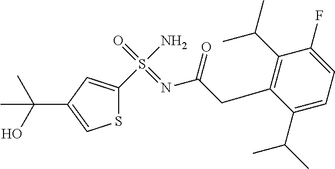 [SMILES: CC(C)c1ccc(F)c(C(C)C)c1CC(=O)N=S(N)(=O)c1cc(C(C)(C)O)cs1] | N-(amino(4-(2- hydroxypropan-2- yl)thiophen-2-yl)(oxo)-λ⁶- sulfaneylidene)-2-(3-fluoro- 2,6- diisopropylphenyl)acetamide | 441 |
| 107 | 255 | 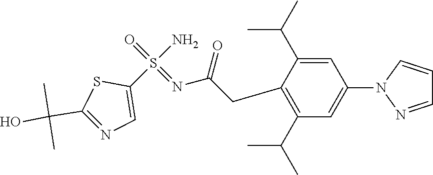 [SMILES: CC(C)c1cc(-n2cccn2)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(2,6- diisopropyl-4-(1H-pyrazol- 1-yl)phenyl)acetamide | 490 |
| 108 | 254 | 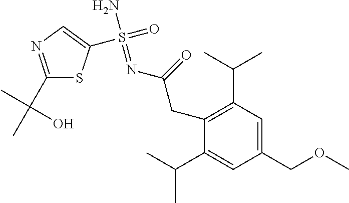 [SMILES: COCc1cc(C(C)C)c(CC(=O)N=S(N)(=O)c2cnc(C(C)(C)O)s2)c(C(C)C)c1] | N-(amino(2-(2- hydroxypropan-2- yl)thiazol-5-yl)(oxo)-λ⁶- sulfaneylidene)-2-(2,6- diisopropyl-4- (methoxymethyl)phenyl) acetamide | 468 |
| 109 | 253 | 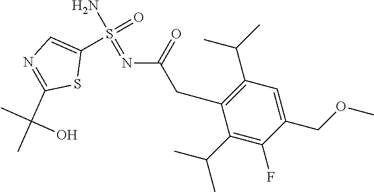 [SMILES: COCc1cc(C(C)C)c(CC(=O)N=S(N)(=O)c2cnc(C(C)(C)O)s2)c(C(C)C)c1F] | N-(amino(2-(2- hydroxypropan-2- yl)thiazol-5-yl)(oxo)-λ⁶- sulfaneylidene)-2-(3-fluoro- 2,6-diisopropyl-4- (methoxymethyl)phenyl) acetamide | 486 |
| 110 | 252 | 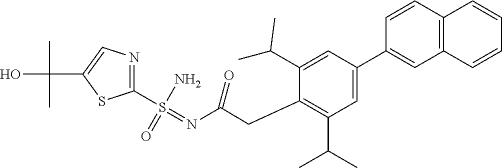 [SMILES: CC(C)c1cc(-c2ccc3ccccc3c2)cc(C(C)C)c1CC(=O)N=S(N)(=O)c1ncc(C(C)(C)O)s1] | N-(amino(5-(2- hydroxypropan-2- yl)thiazol-2-yl)(oxo)-λ⁶- sulfaneylidene)-2-(2,6- diisopropyl-4-(naphthalen- 2-yl)phenyl)acetamide | 550 |
| 111 | 251 | 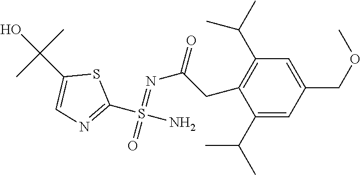 [SMILES: COCc1cc(C(C)C)c(CC(=O)N=S(N)(=O)c2ncc(C(C)(C)O)s2)c(C(C)C)c1] | N-(amino(5-(2- hydroxypropan-2- yl)thiazol-2-yl)(oxo)-λ⁶- sulfaneylidene)-2-(2,6- diisopropyl-4- (methoxymethyl)phenyl) acetamide | 468 |
| 112 | 250 | 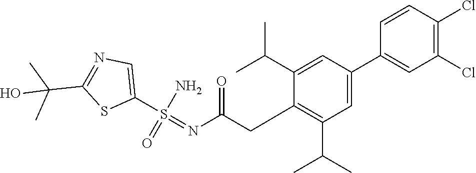 [SMILES: CC(C)c1cc(-c2ccc(Cl)c(Cl)c2)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(3′,4′- dichloro-3,5-diisopropyl- [1,1′-biphenyl]-4- yl)acetamide | 568 |
| 113 | 249 | 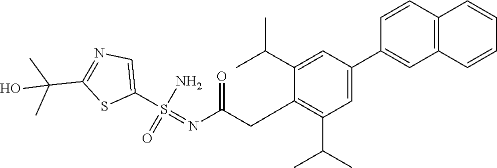 [SMILES: CC(C)c1cc(-c2ccc3ccccc3c2)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(2,6- diisopropyl-4-(naphthalen- 2-yl)phenyl)acetamide | 550 |
| 114 | 248 | 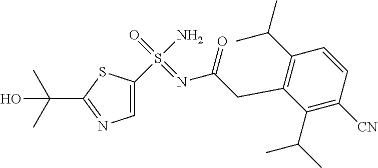 [SMILES: CC(C)c1ccc(C#N)c(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(3-cyano- 2,6- diisopropylphenyl)acetamide | 449 |
| 115 | 247 | 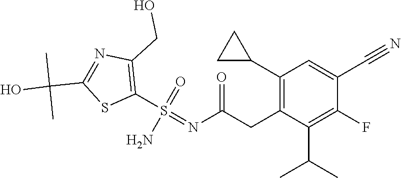 [SMILES: CC(C)c1c(F)c(C#N)cc(C2CC2)c1CC(=O)N=S(N)(=O)c1sc(C(C)(C)O)nc1CO] | N-(amino(4- (hydroxymethyl)-2-(2- hydroxypropan-2- yl)thiazol-5-yl)(oxo)-λ⁶- sulfaneylidene)-2-(4-cyano- 6-cyclopropyl-3-fluoro-2- isopropylphenyl)acetamide | 495 |
| 116 | 246 | 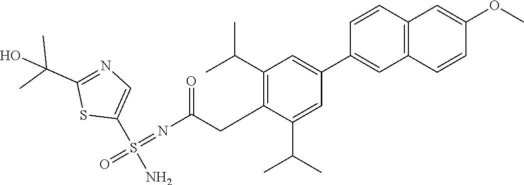 [SMILES: COc1ccc2cc(-c3cc(C(C)C)c(CC(=O)N=S(N)(=O)c4cnc(C(C)(C)O)s4)c(C(C)C)c3)ccc2c1] | N-(amino(2-(2- hydroxypropan-2- yl)thiazol-5-yl)(oxo)-λ⁶- sulfaneylidene)-2-(2,6- diisopropyl-4-(6- methoxynaphthalen-2- yl)phenyl)acetamide | 580 |
| 117 | 309 | 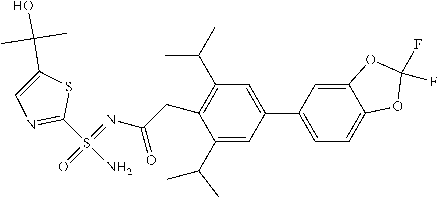 [SMILES: CC(C)c1cc(-c2ccc3c(c2)OC(F)(F)O3)cc(C(C)C)c1CC(=O)N=S(N)(=O)c1ncc(C(C)(C)O)s1] | N-(amino(5-(2- hydroxypropan-2- yl)thiazol-2-yl)(oxo)-λ⁶- sulfaneylidene)-2-(4-(2,2- difluorobenzo[d][1,3]dioxol- 5-yl)-2,6- diisopropylphenyl)acetamide | 580 |
| 118 | 310 | 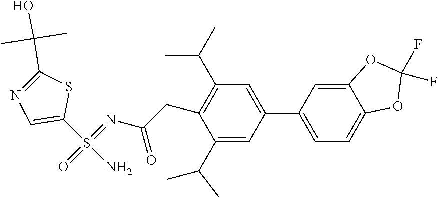 [SMILES: CC(C)c1cc(-c2ccc3c(c2)OC(F)(F)O3)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4-(2,2- difluorobenzo[d][1,3]dioxol- 5-yl)-2,6- diisopropylphenyl)acetamide | 580 |
| 119 | 243 | 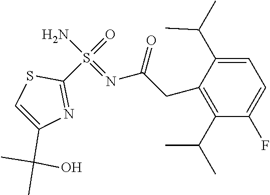 [SMILES: CC(C)c1ccc(F)c(C(C)C)c1CC(=O)N=S(N)(=O)c1nc(C(C)(C)O)cs1] | N-(amino(4-(2- hydroxypropan-2- yl)thiazol-2-yl)(oxo)-λ⁶- sulfaneylidene)-2-(3-fluoro- 2,6- diisopropylphenyl)acetamide | 442 |
| 120 | 242 | 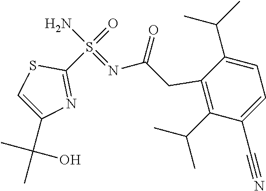 [SMILES: CC(C)c1ccc(C#N)c(C(C)C)c1CC(=O)N=S(N)(=O)c1nc(C(C)(C)O)cs1] | N-(amino(4-(2- hydroxypropan-2- yl)thiazol-2-yl)(oxo)-λ⁶- sulfaneylidene)-2-(3-cyano- 2,6- diisopropylphenyl)acetamide | 449 |
| 121 | 239 | 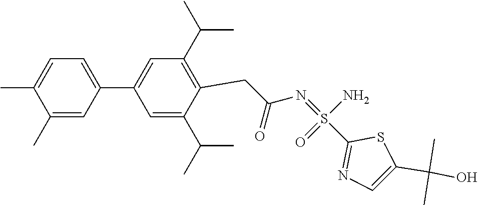 [SMILES: Cc1ccc(-c2cc(C(C)C)c(CC(=O)N=S(N)(=O)c3ncc(C(C)(C)O)s3)c(C(C)C)c2)cc1C] | N-(amino(5-(2- hydroxypropan-2- yl)thiazol-2-yl)(oxo)-λ⁶- sulfaneylidene)-2-(3,5- diisopropyl-3′,4′-dimethyl- [1,1′-biphenyl]-4- yl)acetamide | 528 |
| 122 | 238 | 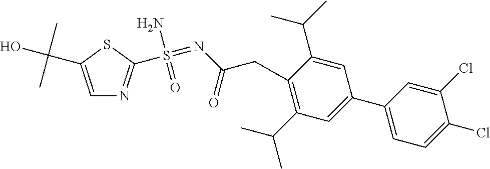 [SMILES: CC(C)c1cc(-c2ccc(Cl)c(Cl)c2)cc(C(C)C)c1CC(=O)N=S(N)(=O)c1ncc(C(C)(C)O)s1] | N-(amino(5-(2- hydroxypropan-2- yl)thiazol-2-yl)(oxo)-λ⁶- sulfaneylidene)-2-(3′,4′- dichloro-3,5-diisopropyl- [1,1′-biphenyl]-4- yl)acetamide | 568 |
| 123 | 237 | 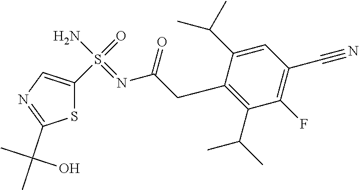 [SMILES: CC(C)c1cc(C#N)c(F)c(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4-cyano- 3-fluoro-2,6- diisopropylphenyl)acetamide | 467 |
| 124 | 236 | 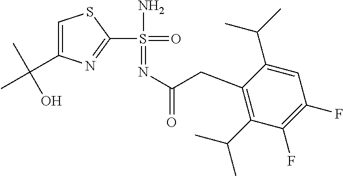 [SMILES: CC(C)c1cc(F)c(F)c(C(C)C)c1CC(=O)N=S(N)(=O)c1nc(C(C)(C)O)cs1] | N-(amino(4-(2- hydroxypropan-2- yl)thiazol-2-yl)(oxo)-λ⁶- sulfaneylidene)-2-(3,4- difluoro-2,6- diisopropylphenyl)acetamide | 460 |
| 125 | 234 | 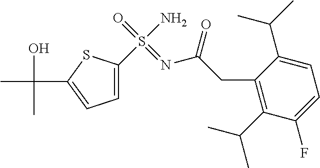 [SMILES: CC(C)c1ccc(F)c(C(C)C)c1CC(=O)N=S(N)(=O)c1ccc(C(C)(C)O)s1] | N-(amino(5-(2- hydroxypropan-2- yl)thiophen-2-yl)(oxo)-λ⁶- sulfaneylidene)-2-(3-fluoro- 2,6- diisopropylphenyl)acetamide | 441 |
| 126 | 233 | 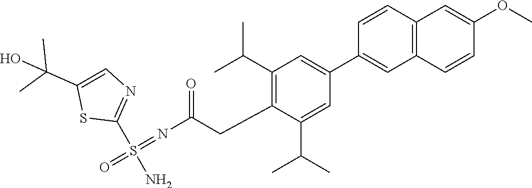 [SMILES: COc1ccc2cc(-c3cc(C(C)C)c(CC(=O)N=S(N)(=O)c4ncc(C(C)(C)O)s4)c(C(C)C)c3)ccc2c1] | N-(amino(5-(2- hydroxypropan-2- yl)thiazol-2-yl)(oxo)-λ⁶- sulfaneylidene)-2-(2,6- diisopropyl-4-(6- methoxynaphthalen-2- yl)phenyl)acetamide | 580 |
| 127 | 232 | 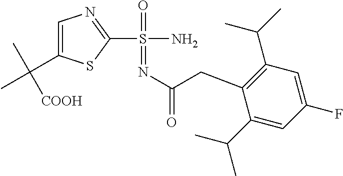 [SMILES: CC(C)c1cc(F)cc(C(C)C)c1CC(=O)N=S(N)(=O)c1ncc(C(C)(C)C(=O)O)s1] | 2-(2-(N′-(2-(4-fluoro-2,6- diisopropylphenyl)acetyl) sulfamidimidoyl)thiazol-5- yl)-2-methylpropanoic acid | 470 |
| 128 | 120 | 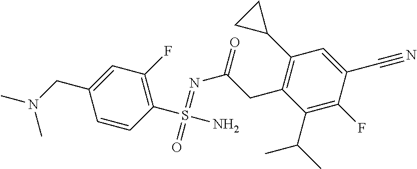 [SMILES: CC(C)c1c(F)c(C#N)cc(C2CC2)c1CC(=O)N=S(N)(=O)c1ccc(CN(C)C)cc1F] | N-(amino(4- ((dimethylamino)methyl)-2- fluorophenyl)(oxo)-λ⁶- sulfaneylidene)-2-(4-cyano- 6-cyclopropyl-3-fluoro-2- isopropylphenyl)acetamide | 475 |
| 129 | 229 | 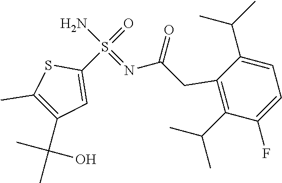 [SMILES: Cc1sc(S(N)(=O)=NC(=O)Cc2c(C(C)C)ccc(F)c2C(C)C)cc1C(C)(C)O] | N-(amino(4-(2- hydroxypropan-2-yl)-5- methylthiophen-2-yl)(oxo)- λ⁶-sulfaneylidene)-2-(3- fluoro-2,6- diisopropyl-phenyl)acetamide | 455 |
| 130 | 228 | 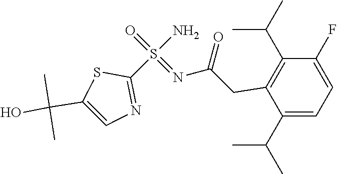 [SMILES: CC(C)c1ccc(F)c(C(C)C)c1CC(=O)N=S(N)(=O)c1ncc(C(C)(C)O)s1] | N-(amino(5-(2- hydroxypropan-2- yl)thiazol-2-yl)(oxo)-λ⁶- sulfaneylidene)-2-(3-fluoro- 2,6- diisopropyl-phenyl)acetamide | 442 |
| 132 | 227 | 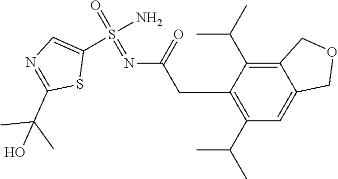 [SMILES: CC(C)c1cc2c(c(C(C)C)c1CC(=O)N=S(N)(=O)c1cnc(C(C)(C)O)s1)COC2] | N-(amino(2-(2- hydroxypropan-2- yl)thiazol-5-yl)(oxo)-λ⁶- sulfaneylidene)-2-(4,6- diisopropyl-1,3- dihydroisobenzofuran-5- yl)acetamide | 466 |
| 133 | 226 | 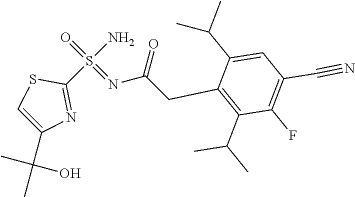 [SMILES: CC(C)c1cc(C#N)c(F)c(C(C)C)c1CC(=O)N=S(N)(=O)c1nc(C(C)(C)O)cs1] | N-(amino(4-(2- hydroxypropan-2- yl)thiazol-2-yl)(oxo)-λ⁶- sulfaneylidene)-2-(4-cyano- 3-fluoro-2,6- diisopropylphenyl)acetamide | 467 |
| 134 | 225 | 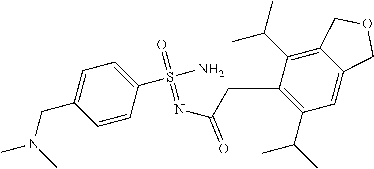 [SMILES: CC(C)c1cc2c(c(C(C)C)c1CC(=O)N=S(N)(=O)c1ccc(CN(C)C)cc1)COC2] | N-(amino(4- ((dimethylamino)methyl) phenyl(oxo)-λ⁶- sulfaneylidene)-2-(4,6- diisopropyl-1,3- dihydroisobenzofuran-5- yl)acetamide | 458 |
| 135 | 224 | 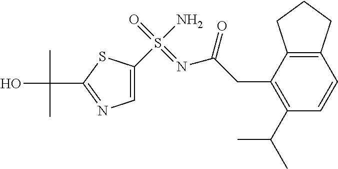 [SMILES: CC(C)c1ccc2c(c1CC(=O)N=S(N)(=O)c1cnc(C(C)(C)O)s1)CCC2] | N-(amino(2-(2- hydroxypropan-2- yl)thiazol-5-yl)(oxo)-λ⁶- sulfaneylidene)-2-(5- isopropyl-2,3-dihydro-1H- inden-4-yl)acetamide | 422 |
| 136 | 191 | 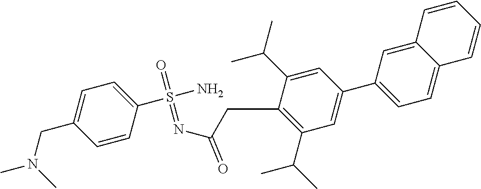 [SMILES: CC(C)c1cc(-c2ccc3ccccc3c2)cc(C(C)C)c1CC(=O)N=S(N)(=O)c1ccc(CN(C)C)cc1] | N-(amino(4- ((dimethylamino)methyl) phenyl)(oxo)-λ⁶- sulfaneylidene)-2-(2,6- diisopropyl-4-(naphthalen- 2-yl)phenyl)acetamide | 542 |
| 137 | 222 | 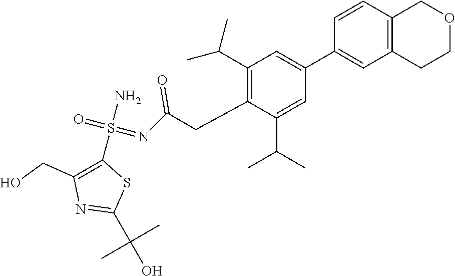 [SMILES: CC(C)c1cc(-c2ccc3c(c2)CCOC3)cc(C(C)C)c1CC(=O)N=S(N)(=O)c1sc(C(C)(C)O)nc1CO] | N-(amino(4- (hydroxymethyl)-2-(2- hydroxypropan-2- yl)thiazol-5-yl)(oxo)-λ⁶- sulfaneylidene)-2-(4- (isochroman-6-yl)-2,6- diisopropyl- phenyl)acetamide | 586 |
| 138 | 307 | 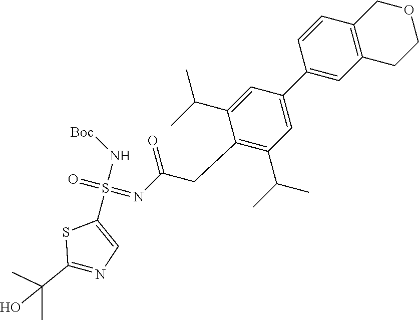 [SMILES: CC(C)c1cc(-c2ccc3c(c2)CCOC3)cc(C(C)C)c1CC(=O)N=S(=O)(NC(=O)OC(C)(C)C)c1cnc(C(C)(C)O)s1] | tert-butyl (2-(2- hydroxypropan-2-yl)-N-(2- (4-(isochroman-6-yl)-2,6- diisopropylphenyl)acetyl) thiazole-5- sulfonimidoyl)carbamate | 656 |
| 139 | 231 | 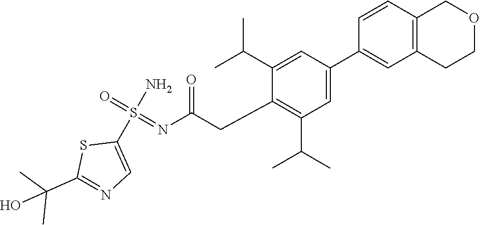 [SMILES: CC(C)c1cc(-c2ccc3c(c2)CCOC3)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4- (isochroman-6-yl)-2,6- diisopropylphenyl)acetamide | 556 |
| 140 | 306 | 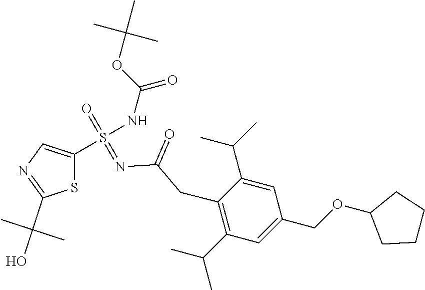 [SMILES: CC(C)c1cc(COC2CCCC2)cc(C(C)C)c1CC(=O)N=S(=O)(NC(=O)OC(C)(C)C)c1cnc(C(C)(C)O)s1] | tert-butyl (N-(2-(4- ((cyclopentyloxy)methyl)- 2,6- diisopropylphenyl)acetyl)- 2-(2-hydroxypropan-2- yl)thiazole-5- sulfonimidoyl)carbamate | 622 |
| 141 | 305 | 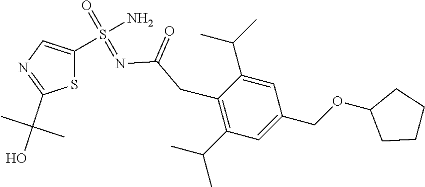 [SMILES: CC(C)c1cc(COC2CCCC2)cc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4- ((cyclopentyloxy)methyl)- 2,6- diisopropyl- phenyl)acetamide | 522 |
| 142 | 216 | 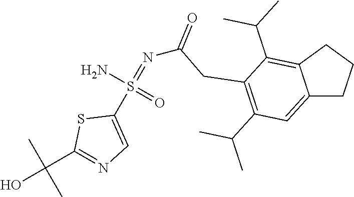 [SMILES: CC(C)c1cc2c(c(C(C)C)c1CC(=O)N=S(N)(=O)c1cnc(C(C)(C)O)s1)CCC2] | N-(amino(2-(2- hydroxypropan-2- yl)thiazol-5-yl)(oxo)-λ⁶- sulfaneylidene)-2-(4,6- diisopropyl-2,3-dihydro- 1H-inden-5-yl)acetamide | 464 |
| 143 | 215 | 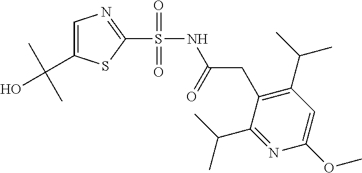 [SMILES: COc1cc(C(C)C)c(CC(=O)NS(=O)(=O)c2ncc(C(C)(C)O)s2)c(C(C)C)n1] | 2-(2,4-diisopropyl-6- methoxypyridin-3-yl)-N- ((5-(2-hydroxypropan-2- yl)thiazol-2- yl)sulfonyl)acetamide | 456 |
| 144 | 203 | 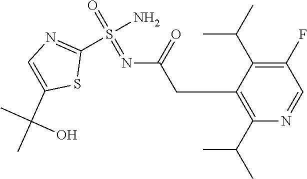 [SMILES: CC(C)c1ncc(F)c(C(C)C)c1CC(=O)N=S(N)(=O)c1ncc(C(C)(C)O)s1] | N-(amino(5-(2- hydroxypropan-2- yl)thiazol-2-yl)(oxo)-λ⁶- sulfaneylidene)-2-(5-fluoro- 2,4-diisopropylpyridin-3- yl)acetamide | 443 |
| 145 | 213 | 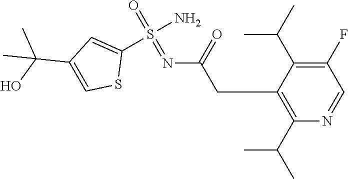 [SMILES: CC(C)c1ncc(F)c(C(C)C)c1CC(=O)N=S(N)(=O)c1cc(C(C)(C)O)cs1] | N-(amino(4-(2- hydroxypropan-2- yl)thiophen-2-yl)(oxo)-λ⁶- sulfaneylidene)-2-(5-fluoro- 2,4-diisopropylpyridin-3- yl)acetamide | 442 |
| 146 | 204 | 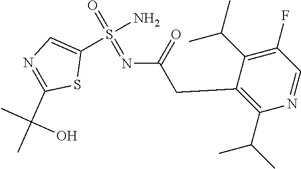 [SMILES: CC(C)c1ncc(F)c(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(5-fluoro- 2,4-diisopropylpyridin-3- yl)acetamide | 443 |
| 147 | 202 | 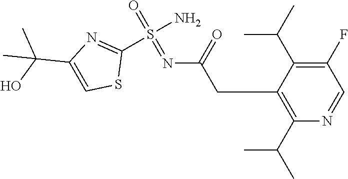 [SMILES: CC(C)c1ncc(F)c(C(C)C)c1CC(=O)N=S(N)(=O)c1nc(C(C)(C)O)cs1] | N-(amino(4-(2- hydroxypropan-2- yl)thiazol-2-yl)(oxo)-λ⁶- sulfaneylidene)-2-(5-fluoro- 2,4-diisopropylpyridin-3- yl)acetamide | 443 |
| 148 | 211 | 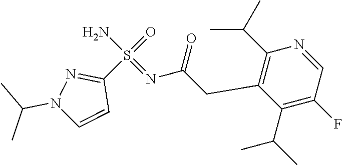 [SMILES: CC(C)c1ncc(F)c(C(C)C)c1CC(=O)N=S(N)(=O)c1ccn(C(C)C)n1] | N-(amino(1-isopropyl-1H- pyrazol-3-yl)(oxo)-λ⁶- sulfaneylidene)-2-(5-fluoro- 2,4-diisopropylpyridin-3- yl)acetamide | 410 |
| 149 | 210 | 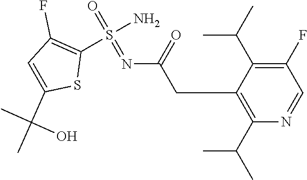 [SMILES: CC(C)c1ncc(F)c(C(C)C)c1CC(=O)N=S(N)(=O)c1sc(C(C)(C)O)cc1F] | N-(amino(3-fluoro-5-(2- hydroxypropan-2- yl)thiophen-2-yl)(oxo)-λ⁶- sulfaneylidene)-2-(5-fluoro- 2,4-diisopropylpyridin-3- yl)acetamide | 460 |
| 150 | 209 | 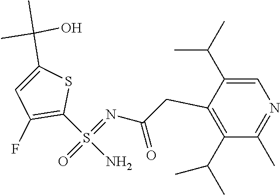 [SMILES: Cc1ncc(C(C)C)c(CC(=O)N=S(N)(=O)c2sc(C(C)(C)O)cc2F)c1C(C)C] | N-(amino(3-fluoro-5-(2- hydroxypropan-2- yl)thiophen-2-yl)(oxo)-λ⁶- sulfaneylidene)-2-(3,5- diisopropyl-2- methylpyridin-4- yl)acetamide | 456 |
| 151 | 208 | 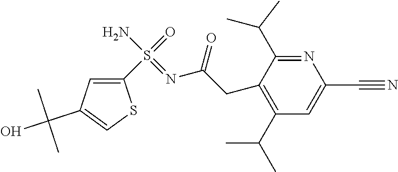 [SMILES: CC(C)c1cc(C#N)nc(C(C)C)c1CC(=O)N=S(N)(=O)c1cc(C(C)(C)O)cs1] | N-(amino(4-(2- hydroxypropan-2- yl)thiophen-2-yl)(oxo)-λ⁶- sulfaneylidene)-2-(6-cyano- 2,4-diisopropylpyridin-3- yl)acetamide | 449 |
| 152 | 207 | 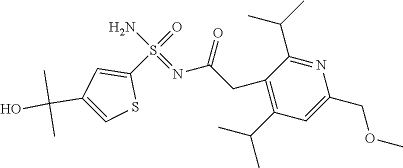 [SMILES: COCc1cc(C(C)C)c(CC(=O)N=S(N)(=O)c2cc(C(C)(C)O)cs2)c(C(C)C)n1] | N-(amino(4-(2- hydroxypropan-2- yl)thiophen-2-yl)(oxo)-λ⁶- sulfaneylidene)-2-(2,4- diisopropyl-6- (methoxymethyl)pyridin-3- yl)acetamide | 468 |
| 153 | 206 | 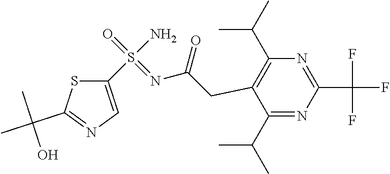 [SMILES: CC(C)c1nc(C(F)(F)F)nc(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(4,6- diisopropyl-2- (trifluoromethyl)pyrimidin- 5-yl)acetamide | 494 |
| 154 | 205 | 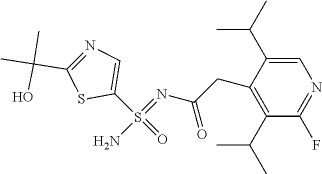 [SMILES: CC(C)c1cnc(F)c(C(C)C)c1CC(=O)N=S(N)(=O)c1cnc(C(C)(C)O)s1] | N-(amino(2-(2- hydroxypropan-2- yl)thiazol-5-yl)(oxo)-λ⁶- sulfaneylidene)-2-(2-fluoro- 3,5-diisopropylpyridin-4- yl)acetamide | 443 |
| 220 | 263 | 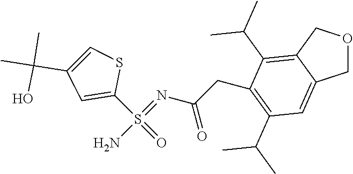 [SMILES: CC(C)c1cc2c(c(C(C)C)c1CC(=O)N=S(N)(=O)c1cc(C(C)(C)O)cs1)COC2] | N-(amino(4-(2- hydroxypropan-2- yl)thiophen-2-yl)(oxo)-λ⁶- sulfaneylidene)-2-(4,6- diisopropyl-1,3- dihydroisobenzofuran-5- yl)acetamide | 465 |
| 221 | 264 | 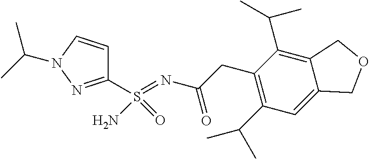 [SMILES: CC(C)c1cc2c(c(C(C)C)c1CC(=O)N=S(N)(=O)c1ccn(C(C)C)n1)COC2] | N-(amino(1-isopropyl-1H- pyrazol-3-yl)(oxo)-λ⁶- sulfaneylidene)-2-(4,6- diisopropyl-1,3- dihydroisobenzofuran-5- yl)acetamide | 433 |

TABLE 22
Examples in the following table were obtained from chiral HPLC resolutions of racemic examples described above. The chiral column and eluents are listed in the table. As a convention, the faster-eluting enantiomer
is always listed first in the table followed by the slower-eluting enantiomer of the pair. The symbol * at a chiral center denotes that this chiral center has been resolved and the absolute stereochemistry
at that center has not been determined. Assigned stereochemistry in compound names are tentative.

| | | | | | | LC-MS |
|---|---|---|---|---|---|---|
| Example # | Compound # | Structure | IUPAC Name | Column | Eluents | [M + H]⁺ |
| 155 | 261b | 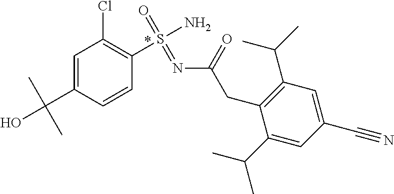 [SMILES: CC(C)c1cc(C#N)cc(C(C)C)c1CC(=O)N=S(N)(=O)c1ccc(C(C)(C)O)cc1Cl] | (R) or (S)-N-(amino(2-chloro- 4-(2-hydroxypropan-2- yl)phenyl)(oxo)-λ⁶- sulfaneylidene)-2-(4-cyano- 2,6- diisopropylphenyl)acetamide | CHIRALPAK ID, 2*25 cm (5 um) | IPA in Hex (0.1% FA) | 476 |
| 156 | 261a | 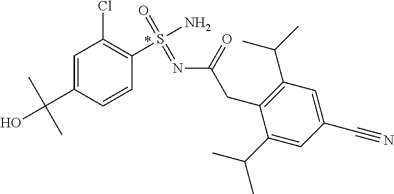 [SMILES: CC(C)c1cc(C#N)cc(C(C)C)c1CC(=O)N=S(N)(=O)c1ccc(C(C)(C)O)cc1Cl] | (S) or (R)-N-(amino(2-chloro- 4-(2-hydroxypropan-2- yl)phenyl)(oxo)-λ⁶- sulfaneylidene)-2-(4-cyano- 2,6- diisopropylphenyl)acetamide | CHIRALPAK ID, 2*25 cm (5 um) | IPA in Hex (0.1% FA) | 476 |
| 157 | 256a | 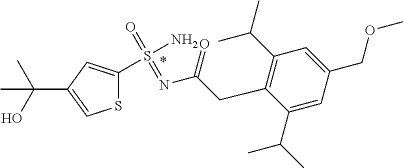 [SMILES: COCc1cc(C(C)C)c(CC(=O)N=S(N)(=O)c2cc(C(C)(C)O)cs2)c(C(C)C)c1] | (R) or (S)-N-(amino(4-(2- hydroxypropan-2-yl)thiophen- 2-yl)(oxo)-λ⁶-sulfaneylidene)- 2-(2,6-diisopropyl-4- (methoxymethyl)phenyl) acetamide | CHIRALPAK ID, 2*25 cm (5 um) | IPA in Hex:DC M = 5:1 | 465 ( M − 1) |
| 158 | 256b | 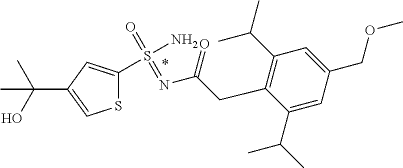 [SMILES: COCc1cc(C(C)C)c(CC(=O)N=S(N)(=O)c2cc(C(C)(C)O)cs2)c(C(C)C)c1] | (S) or (R)-N-(amino(4-(2- hydroxypropan-2-yl)thiophen- 2-yl)(oxo)-λ⁶-sulfaneylidene)- 2-(2,6-diisopropyl-4- (methoxymethyl)phenyl) acetamide | CHIRALPAK ID, 2*25 cm (5 um) | IPA in Hex:DC M = 5:1 | 465 (M − 1) |
| 159 | 201b | 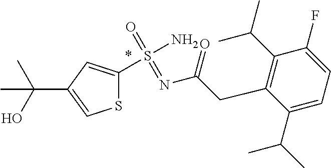 [SMILES: CC(C)c1ccc(F)c(C(C)C)c1CC(=O)N=S(N)(=O)c1cc(C(C)(C)O)cs1] | (R) or (S)-N-(amino(4-(2- hydroxypropan-2-yl)thiophen- 2-yl)(oxo)-λ⁶-sulfaneylidene)- 2-(3-fluoro-2,6- diisopropylphenyl)acetamide | CHIRALPAK ID, 2*25 cm (5 um) | IPA in Hex:DC M = 5:1 | 441 |
| 160 | 201a | 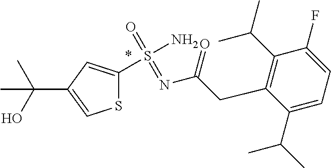 [SMILES: CC(C)c1ccc(F)c(C(C)C)c1CC(=O)N=S(N)(=O)c1cc(C(C)(C)O)cs1] | (S) or (R)-N-(amino(4-(2- hydroxypropan-2-yl)thiophen- 2-yl)(oxo)-λ⁶-sulfaneylidene)- 2-(3-fluoro-2,6- diisopropylphenyl)acetamide | CHIRALPAK ID, 2*25 cm (5 um) | IPA in Hex:DC M = 5:1 | 441 |
| 161 | 258a | 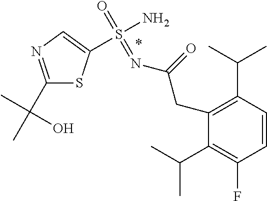 [SMILES: CC(C)c1ccc(F)c(C(C)C)c1CC(=O)N=S(N)(=O)c1cnc(C(C)(C)O)s1] | (R) or (S)-N-(amino(2-(2- hydroxypropan-2-yl)thiazol-5- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 440 (M − 1) |
| 162 | 258b | 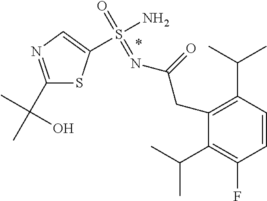 [SMILES: CC(C)c1ccc(F)c(C(C)C)c1CC(=O)N=S(N)(=O)c1cnc(C(C)(C)O)s1] | (S) or (R)-N-(amino(2-(2- hydroxypropan-2-yl)thiazol-5- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 440 (M − 1) |
| 163 | 249a | 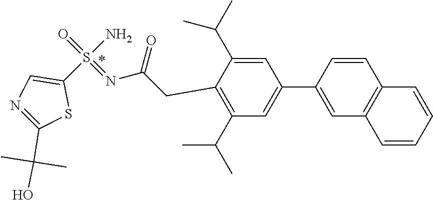 [SMILES: CC(C)c1cc(-c2ccc3ccccc3c2)cc(C(C)C)c1CC(=O)N=S(N)(=O)c1cnc(C(C)(C)O)s1] | (R) or (S)-N-(amino(2-(2- hydroxypropan-2-yl)thiazol-5- yl)(oxo)-λ⁶-sulfaneylidene)-2- (2,6-diisopropyl-4- (naphthalen-2- yl)phenyl)acetamide | Chiralpak ID, 2*25 cm, 5 um | IPA in Hex (0.1% FA) | 550 |
| 164 | 249b | 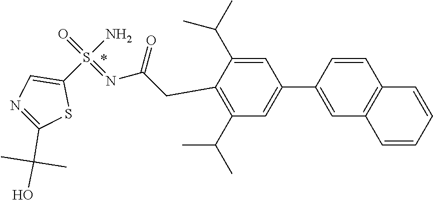 [SMILES: CC(C)c1cc(-c2ccc3ccccc3c2)cc(C(C)C)c1CC(=O)N=S(N)(=O)c1cnc(C(C)(C)O)s1] | (S) or (R)-N-(amino(2-(2- hydroxypropan-2-yl)thiazol-5- yl)(oxo)-λ⁶-sulfaneylidene)-2- (2,6-diisopropyl-4- (naphthalen-2- yl)phenyl)acetamide | Chiralpak ID, 2*25 cm, 5 um | IPA in Hex (0.1% FA) | 550 |
| 165 | 309b | 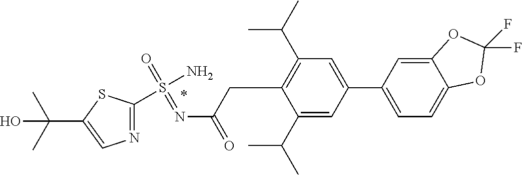 [SMILES: CC(C)c1cc(-c2ccc3c(c2)OC(F)(F)O3)cc(C(C)C)c1CC(=O)N=S(N)(=O)c1ncc(C(C)(C)O)s1] | (S) or (R)-N-(amino(5-(2- hydroxypropan-2-yl)thiazol-2- yl)(oxo)-λ⁶-sulfaneylidene)-2- (4-(2,2- difluorobenzo[d][1,3]dioxol- 5-yl)-2,6- diisopropylphenyl)acetamide | CHIRALPAK IF, 2*25 cm, 5 um | EtOH in Hex (0.1% FA) | 580 |
| 166 | 309a | 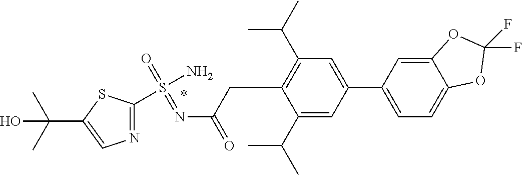 [SMILES: CC(C)c1cc(-c2ccc3c(c2)OC(F)(F)O3)cc(C(C)C)c1CC(=O)N=S(N)(=O)c1ncc(C(C)(C)O)s1] | (R) or (S)-N-(amino(5-(2- hydroxypropan-2-yl)thiazol-2- yl)(oxo)-λ⁶-sulfaneylidene)-2- (4-(2,2- difuorobenzo[d][1,3]dioxol- 5-yl)-2,6- diisopropylphenyl)acetamide | CHIRALPAK IF, 2*25 cm, 5 um | EtOH in Hex (0.1% FA) | 580 |
| 167 | 239a | 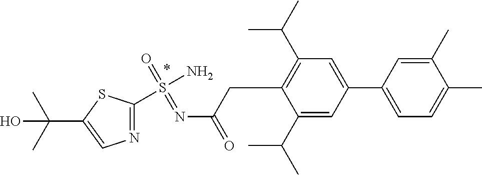 [SMILES: Cc1ccc(-c2cc(C(C)C)c(CC(=O)N=S(N)(=O)c3ncc(C(C)(C)O)s3)c(C(C)C)c2)cc1C] | (S) or (R)-N-(amino(5-(2- hydroxypropan-2-yl)thiazol-2- yl)(oxo)-λ⁶-sulfaneylidene)-2- (3,5-diisopropyl-3′,4′- dimethyl-[1,1′-biphenyl]-4- yl)acetamide | CHIRALPAK IF, 2*25 cm, 5 um | EtOH in Hex (0.1% FA) | 528 |
| 168 | 239b | 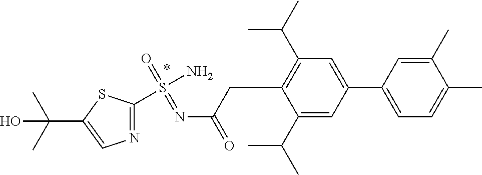 [SMILES: Cc1ccc(-c2cc(C(C)C)c(CC(=O)N=S(N)(=O)c3ncc(C(C)(C)O)s3)c(C(C)C)c2)cc1C] | (R) or (S)-N-(amino(5-(2- hydroxypropan-2-yl)thiazol-2- yl)(oxo)-λ⁶-sulfaneylidene)-2- (3,5-diisopropyl-3′,4′- dimethyl-[1,1′-biphenyl]-4- yl)acetamide | CHIRALPAK IF, 2*25 cm, 5 um | EtOH in Hex (0.1% FA) | 528 |
| 169 | 238a | 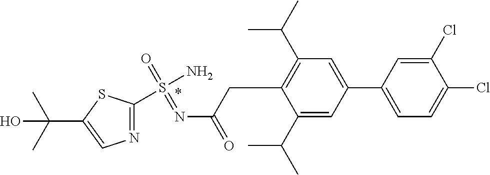 [SMILES: CC(C)c1cc(-c2ccc(Cl)c(Cl)c2)cc(C(C)C)c1CC(=O)N=S(N)(=O)c1ncc(C(C)(C)O)s1] | (S) or (R)-N-(amino(5-(2- hydroxypropan-2-yl)thiazol-2- yl)(oxo)-λ⁶-sulfaneylidene)-2- (3′,4′-dichloro-3,5- diisopropyl-[1,1′-biphenyl]-4- yl)acetamide | CHIRALPAK IF, 2*25 cm, 5 um | IPA in Hex (0.1% FA) | 568 |
| 170 | 238b | 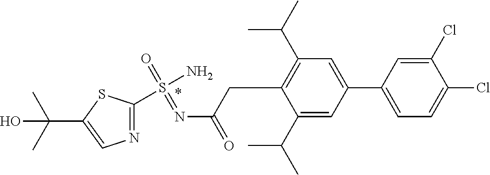 [SMILES: CC(C)c1cc(-c2ccc(Cl)c(Cl)c2)cc(C(C)C)c1CC(=O)N=S(N)(=O)c1ncc(C(C)(C)O)s1] | (R) or (S)-N-(amino(5-(2- hydroxypropan-2-yl)thiazol-2- yl)(oxo)-λ⁶-sulfaneylidene)-2- (3′,4′-dichloro-3,5- diisopropyl-[1,1′-biphenyl]-4- yl)acetamide | CHIRALPAK IF, 2*25 cm, 5 um | IPA in Hex (0.1% FA) | 568 |
| 171 | 221a | 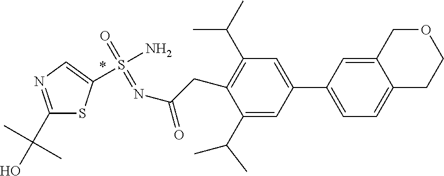 [SMILES: CC(C)c1cc(-c2ccc3c(c2)COCC3)cc(C(C)C)c1CC(=O)N=S(N)(=O)c1cnc(C(C)(C)O)s1] | (R) or (S)-N-(amino(2-(2- hydroxypropan-2-yl)thiazol-5- yl)(oxo)-λ⁶-sulfaneylidene)-2- (4-(isochroman-7-yl)-2,6- diisopropylphenyl)acetamide | CHIRALPAK IF, 2*25 cm, 5 um | EtOH in Hex (0.1% FA) | 556 |
| 172 | 221b | 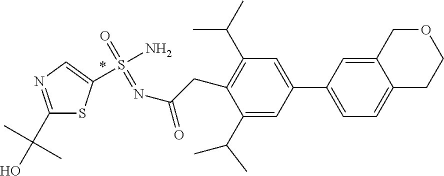 [SMILES: CC(C)c1cc(-c2ccc3c(c2)COCC3)cc(C(C)C)c1CC(=O)N=S(N)(=O)c1cnc(C(C)(C)O)s1] | (S) or (R)-N-(amino(2-(2- hydroxypropan-2-yl)thiazol-5- yl)(oxo)-λ⁶-sulfaneylidene)-2- (4-(isochroman-7-yl)-2,6- diisopropylphenyl)acetamide | CHIRALPAK IF, 2*25 cm, 5 um | EtOH in Hex (0.1% FA) | 556 |
| 173 | 229a | 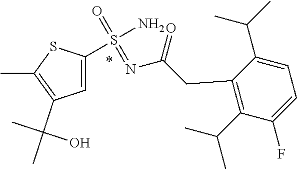 [SMILES: Cc1sc(S(N)(=O)=NC(=O)Cc2c(C(C)C)ccc(F)c2C(C)C)cc1C(C)(C)O] | (R) or (S)-N-(amino(4-(2- hydroxypropan-2-yl)-5- methylthiophen-2-yl)(oxo)- λ⁶-sulfaneylidene)-2-(3- fluoro-2,6- diisopropylphenyl)acetamide | CHIRALPAK IC, 2*25 cm, 5 um | IPA in Hex (0.1% FA) | 455 |
| 174 | 229b | 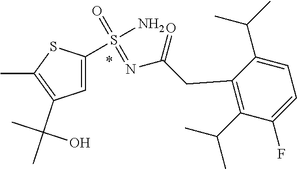 [SMILES: Cc1sc(S(N)(=O)=NC(=O)Cc2c(C(C)C)ccc(F)c2C(C)C)cc1C(C)(C)O] | (S) or (R)-N-(amino(4-(2- hydroxypropan-2-yl)-5- methylthiophen-2-yl)(oxo)-λ⁶- sulfaneylidene)-2-(3-fluoro- 2,6- diisopropylphenyl)acetamide | CHIRALPAK IC, 2*25 cm, 5 um | IPA in Hex (0.1% FA) | 455 |
| 175 | 234b | 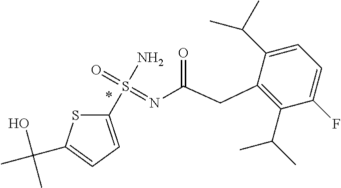 [SMILES: CC(C)c1ccc(F)c(C(C)C)c1CC(=O)N=S(N)(=O)c1ccc(C(C)(C)O)s1] | (S) or (R)-N-(amino(5-(2- hydroxypropan-2-yl)thiophen- 2-yl)(oxo)-λ⁶-sulfaneylidene)- 2-(3-fluoro-2,6- diisopropylphenyl)acetamide | Chiralpak ID, 2*25 cm, 5 um | IPA in Hex (0.1% FA) | 441 |
| 176 | 234a | 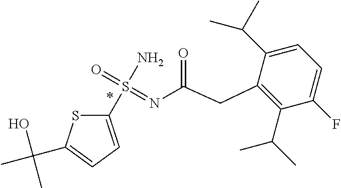 [SMILES: CC(C)c1ccc(F)c(C(C)C)c1CC(=O)N=S(N)(=O)c1ccc(C(C)(C)O)s1] | (R) or (S)-N-(amino(5-(2- hydroxypropan-2-yl)thiophen- 2-yl)(oxo)-λ⁶-sulfaneylidene)- 2-(3-fluoro-2,6- diisopropylphenyl)acetamide | Chiralpak ID, 2*25 cm, 5 um | IPA in Hex (0.1% FA) | 441 |
| 177 | 236a | 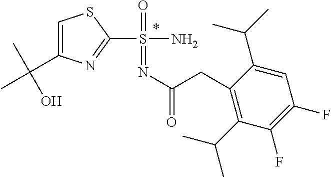 [SMILES: CC(C)c1cc(F)c(F)c(C(C)C)c1CC(=O)N=S(N)(=O)c1nc(C(C)(C)O)cs1] | (R) or (S)-N-(amino(4-(2- hydroxypropan-2-yl)thiazol-2- yl)(oxo)-λ⁶-sulfaneylidene)-2- (3,4-difluoro-2,6- diisopropylphenyl)acetamide | Chiralpak ID, 2*25 cm, 5 um | IPA in Hex (0.1% FA) | 460 |
| 178 | 236b | 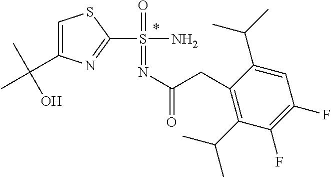 [SMILES: CC(C)c1cc(F)c(F)c(C(C)C)c1CC(=O)N=S(N)(=O)c1nc(C(C)(C)O)cs1] | (S) or (R)-N-(amino(4-(2- hydroxypropan-2-yl)thiazol-2- yl)(oxo)-λ⁶-sulfaneylidene)-2- (3,4-difluoro-2,6- diisopropyiphenyl)acetamide | Chiralpak ID, 2*25 cm, 5 um | IPA in Hex (0.1% FA) | 460 |
| 179 | 242a | 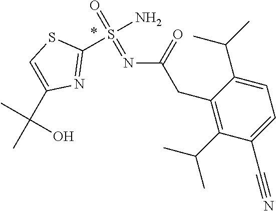 [SMILES: CC(C)c1ccc(C#N)c(C(C)C)c1CC(=O)N=S(N)(=O)c1nc(C(C)(C)O)cs1] | (R) or (S)-N-(amino(4-(2- hydroxypropan-2-yl)thiazol-2- yl)(oxo)-λ⁶-sulfaneylidene)-2- (3-cyano-2,6- diisopropylphenyl)acetamide | Chiralpak ID, 2*25 cm, 5 um | IPA in Hex (0.1% FA) | 449 |
| 180 | 242b | 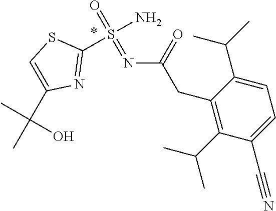 [SMILES: CC(C)c1ccc(C#N)c(C(C)C)c1CC(=O)N=S(N)(=O)c1nc(C(C)(C)O)cs1] | (S) or (R)-N-(amino(4-(2- hydroxypropan-2-yl)thiazol-2- yl)(oxo)-λ⁶-sulfaneylidene)-2- (3-cyano-2,6- diisopropylphenyl)acetamide | Chiralpak ID, 2*25 cm, 5 um | IPA in Hex (0.1% FA) | 449 |
| 181 | 243a | 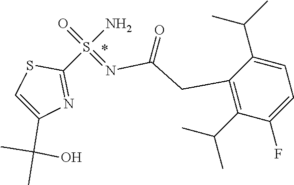 [SMILES: CC(C)c1ccc(F)c(C(C)C)c1CC(=O)N=S(N)(=O)c1nc(C(C)(C)O)cs1] | (R) or (S)-N-(amino(4-(2- hydroxypropan-2-yl)thiazol-2- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 442 |
| 182 | 243b | 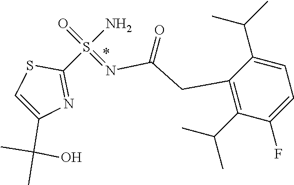 [SMILES: CC(C)c1ccc(F)c(C(C)C)c1CC(=O)N=S(N)(=O)c1nc(C(C)(C)O)cs1] | (S) or (R)-N-(amino(4-(2- hydroxypropan-2-yl)thiazol-2- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 442 |
| 183 | 227a | 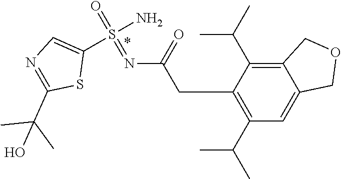 [SMILES: CC(C)c1cc2c(c(C(C)C)c1CC(=O)N=S(N)(=O)c1cnc(C(C)(C)O)s1)COC2] | (R) or (S)-N-(amino(2-(2- hydroxypropan-2-yl)thiazol-5- yl)(oxo)-λ⁶-sulfaneylidene)-2- (4,6-diisopropyl-1,3- dihydroisobenzofuran-5- yl)acetamide | Chiralpak IA, 2*25 cm, 5 um | EtOH in Hex (0.1% FA) | 466 |
| 184 | 227b | 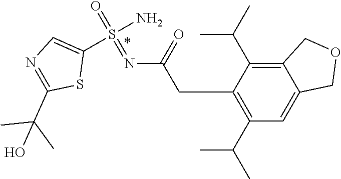 [SMILES: CC(C)c1cc2c(c(C(C)C)c1CC(=O)N=S(N)(=O)c1cnc(C(C)(C)O)s1)COC2] | (S) or (R)-N-(amino(2-(2- hydroxypropan-2-yl)thiazol-5- yl)(oxo)-λ⁶-sulfaneylidene)-2- (4,6-diisopropyl-1,3- dihydroisobenzofuran-5- yl)acetamide | Chiralpak IA, 2*25 cm, 5 um | EtOH in Hex (0.1% FA) | 466 |
| 185 | 251b | 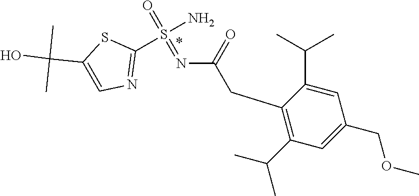 [SMILES: COCc1cc(C(C)C)c(CC(=O)N=S(N)(=O)c2ncc(C(C)(C)O)s2)c(C(C)C)c1] | (R) or (S)-N-(amino(5-(2- hydroxypropan-2-yl)thiazol-2- yl)(oxo)-λ⁶-sulfaneylidene)-2- (2,6-diisopropyl-4- (methoxymethyl)phenyl) acetamide | Chiralpak ID, 2*25 cm, 5 um | IPA in Hex (0.1% FA) | 468 |
| 186 | 251a | 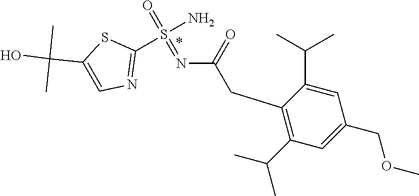 [SMILES: COCc1cc(C(C)C)c(CC(=O)N=S(N)(=O)c2ncc(C(C)(C)O)s2)c(C(C)C)c1] | (S) or (R)-N-(amino(5-(2- hydroxypropan-2-yl)thiazol-2- yl)(oxo)-λ⁶-sulfaneylidene)-2- (2,6-diisopropyl-4- (methoxymethyl)phenyl) acetamide | Chiralpak ID, 2*25 cm, 5 um | IPA in Hex (0.1% FA) | 468 |
| 187 | 228a | 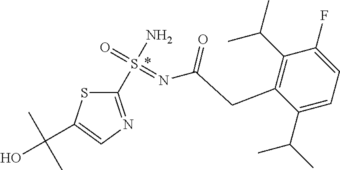 [SMILES: CC(C)c1ccc(F)c(C(C)C)c1CC(=O)N=S(N)(=O)c1ncc(C(C)(C)O)s1] | (S) or (R)-N-(amino(5-(2- hydroxypropan-2-yl)thiazol-2- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 442 |
| 188 | 228b | 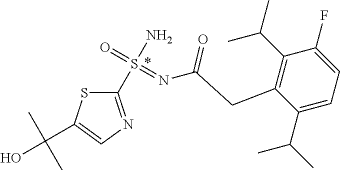 [SMILES: CC(C)c1ccc(F)c(C(C)C)c1CC(=O)N=S(N)(=O)c1ncc(C(C)(C)O)s1] | (R) or (S)-N-(amino(5-(2- hydroxypropan-2-yl)thiazol-2- yl)(oxo)-λ⁶-sulfaneylidene)-2- (3-fluoro-2,6- diisopropylphenyl)acetamide | Chiralpak ID, 2*25 cm, 5 um | IPA in Hex (0.1% FA) | 442 |
| 189 | 226a | 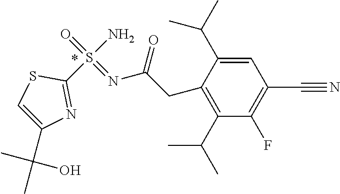 [SMILES: CC(C)c1cc(C#N)c(F)c(C(C)C)c1CC(=O)N=S(N)(=O)c1nc(C(C)(C)O)cs1] | (S) or (R)-N-(amino(4-(2- hydroxypropan-2-yl)thiazol-2- yl)(oxo)-λ⁶-sulfaneylidene)-2- (4-cyano-3-fluoro-2,6- diisopropylphenyl)acetamide | CHIRALPAK IG, 20*250 mm, 5 um | IPA in Hex (0.1% FA) | 467 |
| 190 | 226b | 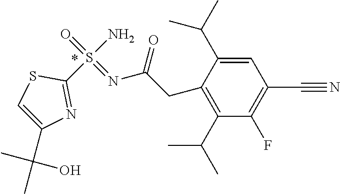 [SMILES: CC(C)c1cc(C#N)c(F)c(C(C)C)c1CC(=O)N=S(N)(=O)c1nc(C(C)(C)O)cs1] | (R) or (S)-N-(amino(4-(2- hydroxypropan-2-yl)thiazol-2- yl)(oxo)-λ⁶-sulfaneylidene)-2- (4-cyano-3-fluoro-2,6- diisopropylphenyl)acetamide | CHIRALPAK IG, 20*250 mm, 5 um | IPA in Hex (0.1% FA) | 467 |
| 191 | 225a | 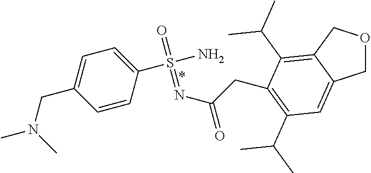 [SMILES: CC(C)c1cc2c(c(C(C)C)c1CC(=O)N=S(N)(=O)c1ccc(CN(C)C)cc1)COC2] | (R) or (S)-N-(amino(4- ((dimethylamino)methyl)phenyl) (oxo)-λ⁶-sulfaneylidene)-2- (4,6-diisopropyl-1,3- dihydroisobenzofuran-5- yl)acetamide | Chiralpak ID, 2*25 cm, 5 um | EtOH in Hex (8 mM NH₃•MeOH) | 458 |
| 192 | 225b | 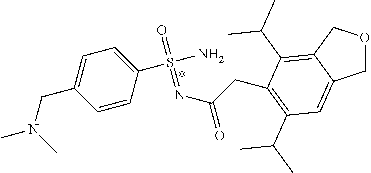 [SMILES: CC(C)c1cc2c(c(C(C)C)c1CC(=O)N=S(N)(=O)c1ccc(CN(C)C)cc1)COC2] | (S) or (R)-N-(amino(4- ((dimethylamino)methyl)phenyl) (oxo)-λ⁶-sulfaneylidene)-2- (4,6-diisopropyl-1,3- dihydroisobenzofuran-5- yl)acetamide | Chiralpak ID, 2*25 cm, 5 um | EtOH in Hex (8 mM NH₃•MeOH) | 458 |
| 193 | 224b | 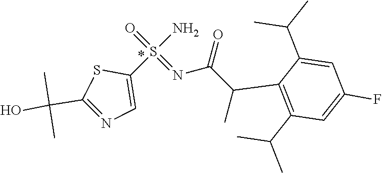 [SMILES: CC(C)c1cc(F)cc(C(C)C)c1C(C)C(=O)N=S(N)(=O)c1cnc(C(C)(C)O)s1] | (S, S) and (S, R)-N-(amino(2- (2-hydroxypropan-2- yl)thiazol-5-yl(oxo)-λ⁶- sulfaneylidene)-2-(4-fluoro- 2,6- diisopropylphenyl) propanamide | Chiralpak IA, 2*25 cm, 5 um | IPA in Hex (0.1% FA) | 456 |
| 194 | 244ba | 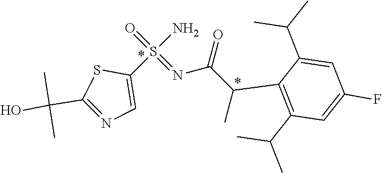 [SMILES: CC(C)c1cc(F)cc(C(C)C)c1C(C)C(=O)N=S(N)(=O)c1cnc(C(C)(C)O)s1] | (R, S) or (R, R)-N-(amino(2- (2-hydroxypropan-2- yl)thiazol-5-yl)(oxo)-λ⁶- sulfaneylidene)-2-(4-fluoro- 2,6- diisopropylphenyl) propanamide | Chiralpak IA, 2*25 cm, 5 um | IPA in Hex (0.1% FA) | 456 |
| 195 | 244aa | 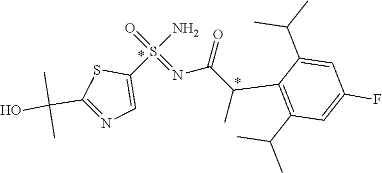 [SMILES: CC(C)c1cc(F)cc(C(C)C)c1C(C)C(=O)N=S(N)(=O)c1cnc(C(C)(C)O)s1] | (R, R) or (R, S)-N-(amino(2- (2-hydroxypropan-2- yl)thiazol-5-yl)(oxo)-λ⁶- sulfaneylidene)-2-(4-fluoro- 2,6- diisopropylphenyl) propanamide | Chiralpak IA, 2*25 cm, 5 um | IPA in Hex (0.1% FA) | 456 |
| 196 | 220a | 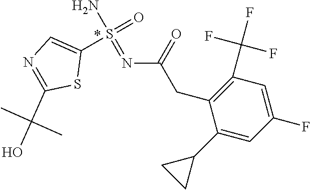 [SMILES: CC(C)(O)c1ncc(S(N)(=O)=NC(=O)Cc2c(C3CC3)cc(F)cc2C(F)(F)F)s1] | (R) or (S)-N-(amino(2-(2- hydroxypropan-2-yl)thiazol-5- yl)(oxo)-λ⁶-sulfaneylidene)-2- (2-cyclopropyl-4-fluoro-6- (trifluoromethyl)phenyl) acetamide | Chiralpak IC, 2*25 cm, 5 um | EtOH in Hex (0.1% FA) | 466 |
| 197 | 220b | 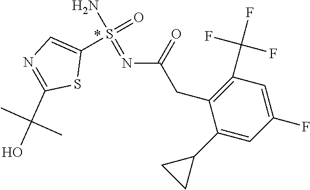 [SMILES: CC(C)(O)c1ncc(S(N)(=O)=NC(=O)Cc2c(C3CC3)cc(F)cc2C(F)(F)F)s1] | (S) or (R)-N-(amino(2-(2- hydroxypropan-2-yl)thiazol-5- yl)(oxo)-λ⁶-sulfaneylidene)-2- (2-cyclopropyl-4-fluoro-6- (trifluoromethyl)phenyl) acetamide | Chiralpak IC, 2*25 cm, 5 um | EtOH in Hex (0.1% FA) | 466 |
| 198 | 218a | 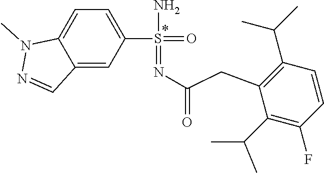 [SMILES: CC(C)c1ccc(F)c(C(C)C)c1CC(=O)N=S(N)(=O)c1ccc2c(cnn2C)c1] | (R) or (S)-N-(amino(1- methyl-1H-indazol-5- yl)(oxo)-λ⁶-sulfaneylidene)-2- (3-fluoro-2,6- diisopropylphenyl)acetamide | CHIRALPAK IG, 2*25 cm, (5 um) | EtOH in Hex (8 mM NH₃•MeOH) | 431 |
| 199 | 218b | 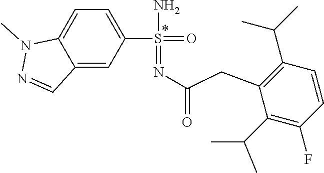 [SMILES: CC(C)c1ccc(F)c(C(C)C)c1CC(=O)N=S(N)(=O)c1ccc2c(cnn2C)c1] | (S) or (R)-N-(amino(1- methyl-1H-indazol-5- yl)(oxo)-λ⁶-sulfaneylidene)-2- (3-fluoro-2,6- diisopropylphenyl)acetamide | CHIRALPAK IG, 2*25 cm, (5 um) | EtOH in Hex (8 mM NH₃•MeOH) | 431 |
| 200 | 217a | 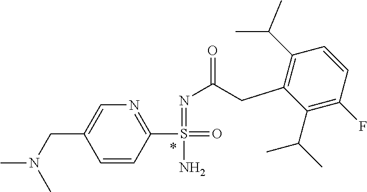 [SMILES: CC(C)c1ccc(F)c(C(C)C)c1CC(=O)N=S(N)(=O)c1ccc(CN(C)C)cn1] | (R) or (S)-N-(amino(5- ((dimethylamino)methyl) pyridin-2-yl)(oxo)-λ⁶- sulfaneylidene)-2-(3-fluoro- 2,6- diisopropylphenyl)acetamide | CHIRALPAK ID, 2*25 cm, (5 um) | IPA in Hex (8 mM NH₃•MeOH) | 435 |
| 201 | 217b | 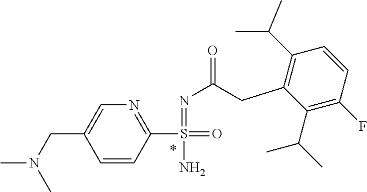 [SMILES: CC(C)c1ccc(F)c(C(C)C)c1CC(=O)N=S(N)(=O)c1ccc(CN(C)C)cn1] | (S) or (R)-N-(amino(5- ((dimethylamino)methyl) pyridin-2-yl)(oxo)-λ⁶- sulfaneylidene)-2-(3-fluoro- 2,6- diisopropylphenyl)acetamide | CHIRALPAK ID, 2*25 cm, (5 um) | IPA in Hex (8 mM NH₃•MeOH) | 435 |
| 202 | 303 | 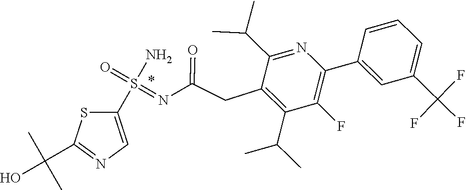 [SMILES: CC(C)c1nc(-c2cccc(C(F)(F)F)c2)c(F)c(C(C)C)c1CC(=O)N=S(N)(=O)c1cnc(C(C)(C)O)s1] | N-(amino(2-(2- hydroxypropan-2-yl)thiazol-5- yl)(oxo)-λ⁶-sulfaneylidene)-2- (5-fluoro-2,4-diisopropyl-6- (3- (trifluoromethyl)phenyl) pyridin-3-yl)acetamide | Chiralpak ID, 2*25 cm, 5 um | IPA in Hex (0.1% FA) | 587 |
| 203 | 302 | 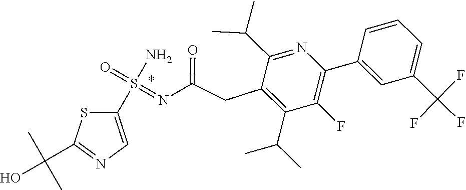 [SMILES: CC(C)c1nc(-c2cccc(C(F)(F)F)c2)c(F)c(C(C)C)c1CC(=O)N=S(N)(=O)c1cnc(C(C)(C)O)s1] | (S) or (R)-N-(amino(2-(2- hydroxypropan-2-yl)thiazol-5- yl)(oxo)-λ⁶-sulfaneylidene)-2- (5-fluoro-2,4-diisopropyl-6- (3- (trifluoromethyl)phenyl) pyridin-3-yl)acetamide | Chiralpak ID, 2*25 cm, 5 um | IPA in Hex (0.1% FA) | 587 |
| 204 | 203b | 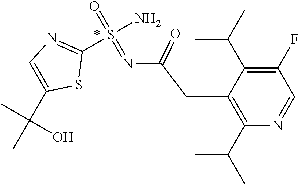 [SMILES: CC(C)c1ncc(F)c(C(C)C)c1CC(=O)N=S(N)(=O)c1ncc(C(C)(C)O)s1] | N-(amino(5-(2- hydroxypropan-2-yl)thiazol-2- yl)(oxo)-λ⁶-sulfaneylidene)-2- (5-fluoro-2,4- diisopropylpyridin-3- yl)acetamide | CHIRALPAK IF, 2*25 cm, 5 um | EtOH in Hex (0.1% FA) | 443 |
| 205 | 203a | 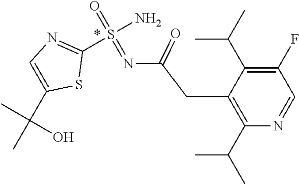 [SMILES: CC(C)c1ncc(F)c(C(C)C)c1CC(=O)N=S(N)(=O)c1ncc(C(C)(C)O)s1] | (S) or (R)-N-(amino(5-(2- hydroxypropan-2-yl)thiazol-2- yl)(oxo)-λ⁶-sulfaneylidene)-2- (5-fluoro-2,4- diisopropylpyridin-3- yl)acetamide | CHIRALPAK IF, 2*25 cm, 5 um | EtOH in Hex (0.1% FA) | 443 |
| 206 | 202a | 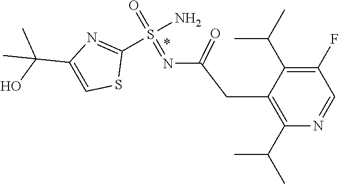 [SMILES: CC(C)c1ncc(F)c(C(C)C)c1CC(=O)N=S(N)(=O)c1nc(C(C)(C)O)cs1] | N-(amino(4-(2- hydroxypropan-2-yl)thiazol-2- yl)(oxo)-λ⁶-sulfaneylidene)-2- (5-fluoro-2,4- diisopropylpyridin-3- yl)acetamide | Chiralpak ID, 2*25 cm, 5 um | IPA in Hex (0.1% FA) | 443 |
| 207 | 202b | 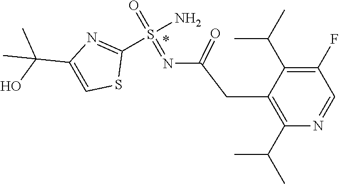 [SMILES: CC(C)c1ncc(F)c(C(C)C)c1CC(=O)N=S(N)(=O)c1nc(C(C)(C)O)cs1] | (S) or (R)-N-(amino(4-(2- hydroxypropan-2-yl)thiazol-2- yl)(oxo)-λ⁶-sulfaneylidene)-2- (5-fluoro-2,4- diisopropylpyridin-3- yl)acetamide | Chiralpak ID, 2*25 cm, 5 um | IPA in Hex (0.1% FA) | 443 |
| 208 | 213a | 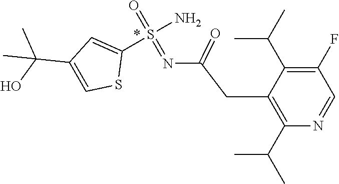 [SMILES: CC(C)c1ncc(F)c(C(C)C)c1CC(=O)N=S(N)(=O)c1cc(C(C)(C)O)cs1] | N-(amino(4-(2- hydroxypropan-2-yl)thiophen- 2-yl)(oxo)-λ⁶-sulfaneylidene)- 2-(5-fIuoro-2,4- diisopropylpyridin-3- yl)acetamide | Chiralpak ID, 2*25 cm, 5 um | IPA in Hex (0.1% FA) | 442 |
| 209 | 213b | 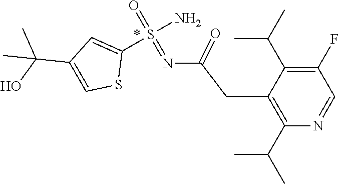 [SMILES: CC(C)c1ncc(F)c(C(C)C)c1CC(=O)N=S(N)(=O)c1cc(C(C)(C)O)cs1] | (S) or (R)-N-(amino(4-(2- hydroxypropan-2-yl)thiophen- 2-yl)(oxo)-λ⁶-sulfaneylidene)- 2-(5-fluoro-2,4- diisopropylpyridin-3- yl)acetamide | Chiralpak ID, 2*25 cm, 5 um | IPA in Hex (0.1% FA) | 442 |
| 210 | 210a | 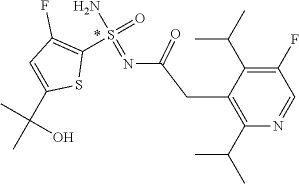 [SMILES: CC(C)c1ncc(F)c(C(C)C)c1CC(=O)N=S(N)(=O)c1sc(C(C)(C)O)cc1F] | (S) or (R)-N-(amino(3-fluoro- 5-(2-hydroxypropan-2- yl)thiophen-2-yl)(oxo)-λ⁶- sulfaneylidene)-2-(5-fluoro- 2,4-diisopropylpyridin-3- yl)acetamide | CHIRALPAK IG, 20*250 mm, 5 um | IPA in Hex (0.1% FA) | 460 |
| 211 | 210b | 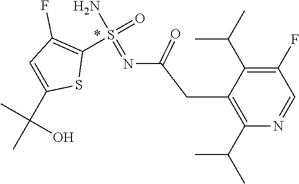 [SMILES: CC(C)c1ncc(F)c(C(C)C)c1CC(=O)N=S(N)(=O)c1sc(C(C)(C)O)cc1F] | N-(amino(3-fluoro-5-(2- hydroxypropan-2-yl)thiophen- 2-yl)(oxo)-λ⁶-sulfaneylidene)- 2-(5-fluoro-2,4- diisopropylpyridin-3- yl)acetamide | CHIRALPAK IG, 20*250 mm, 5 um | IPA in Hex (0.1% FA) | 460 |
| 212 | 216a | 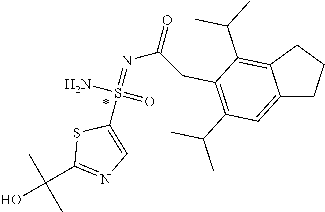 [SMILES: CC(C)c1cc2c(c(C(C)C)c1CC(=O)N=S(N)(=O)c1cnc(C(C)(C)O)s1)CCC2] | (R) or (S)-N-(amino(2-(2- hydroxypropan-2-yl)thiazol-5- yl)(oxo)-λ⁶-sulfaneylidene)-2- (4,6-diisopropyl-2,3-dihydro- 1H-inden-5-yl)acetamide | Chiralpak ID, 2*25 cm, 5 um | EtOH in MTBE (0.1% FA) | 464 |
| 213 | 216b | 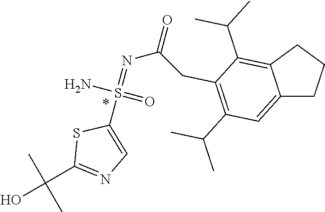 [SMILES: CC(C)c1cc2c(c(C(C)C)c1CC(=O)N=S(N)(=O)c1cnc(C(C)(C)O)s1)CCC2] | (S) or (R)-N-(amino(2-(2- hydroxypropan-2-yl)thiazol-5- yl)(oxo)-λ⁶-sulfaneylidene)-2- (4,6-diisopropyl-2,3-dihydro- 1H-inden-5-yl)acetamide | Chiralpak ID, 2*25 cm, 5 um | EtOH in MTBE (0.1% FA) | 464 |
| 214 | 110a′ | 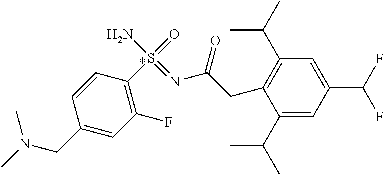 [SMILES: CC(C)c1cc(C(F)F)cc(C(C)C)c1CC(=O)N=S(N)(=O)c1ccc(CN(C)C)cc1F] | (R) or (S)-N-(amino(4- ((dimethylamino)methyl)-2- fluorophenyl)(oxo)-λ⁶- sulfaneylidene)-2-(4- (difluoromethyl)-2,6- diisopropylphenyl)acetamide | CHIRALPAK IG, 2*25 cm, (5 um) | EtOH in Hex (8 mM NH₃•MeOH) | 484 |
| 215 | 110b′ | 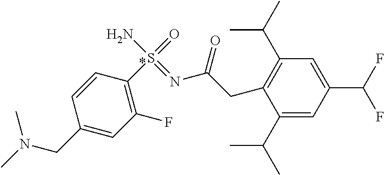 [SMILES: CC(C)c1cc(C(F)F)cc(C(C)C)c1CC(=O)N=S(N)(=O)c1ccc(CN(C)C)cc1F] | (S) or (R)-N-(amino(4- ((dimethylamino)methyl)-2- fluorophenyl)(oxo)-λ⁶- sulfaneylidene)-2-(4- (difluoromethyl)-2,6- diisopropylphenyl)acetamide | CHIRALPAK IG, 2*25 cm, (5 um) | EtOH in Hex (8 mM NH₃•MeOH) | 484 |
| 216 | 263a | 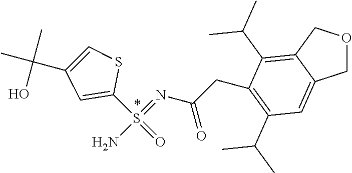 [SMILES: CC(C)c1cc2c(c(C(C)C)c1CC(=O)N=S(N)(=O)c1cc(C(C)(C)O)cs1)COC2] | (R) or (S)-N-(amino(4-(2- hydroxypropan-2-yl)thiophen- 2-yl)(oxo)-λ⁶-sulfaneylidene)- 2-(4,6-diisopropyl-1,3- dihydroisobenzofuran-5- yl)acetamide | CHIRALPAK IG, 2*25 cm, (5 um) | EtOH in Hex (8 mM NH₃•MeOH) | 465 |
| 217 | 263b | 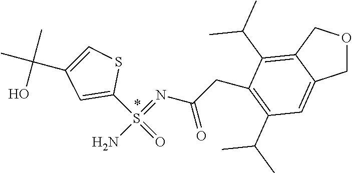 [SMILES: CC(C)c1cc2c(c(C(C)C)c1CC(=O)N=S(N)(=O)c1cc(C(C)(C)O)cs1)COC2] | (S) or (R)-N-(amino(4-(2- hydroxypropan-2-yl)thiophen- 2-yl)(oxo)-λ⁶-sulfaneylidene)- 2-(4,6-diisopropyl-1,3- dihydroisobenzofuran-5- yl)acetamide | CHIRALPAK IG, 2*25 cm, (5 um) | EtOH in Hex (8 mM NH₃•MeOH) | 465 |
| 218 | 264a | 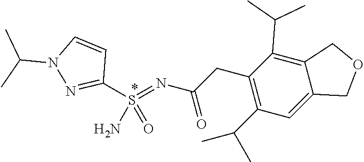 [SMILES: CC(C)c1cc2c(c(C(C)C)c1CC(=O)N=S(N)(=O)c1ccn(C(C)C)n1)COC2] | (R) or (S)-N-(amino(1- isopropyl-1H-pyrazol-3- yl)(oxo)-λ⁶-sulfaneylidene)-2- (4,6-diisopropyl-1,3- dihydroisobenzofuran-5- yl)acetamide | Chiralpak ID, 2*25 cm, 5 um | EtOH in Hex (0.1% FA) | 433 |
| 219 | 264b | 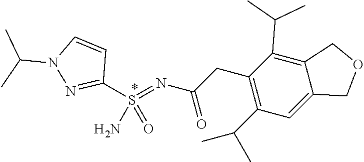 [SMILES: CC(C)c1cc2c(c(C(C)C)c1CC(=O)N=S(N)(=O)c1ccn(C(C)C)n1)COC2] | (S) or (R)-N-(amino(1- isopropyl-1H-pyrazol-3- yl)(oxo)-λ⁶-sulfaneylidene)-2- (4,6-diisopropyl-1,3- dihydroisobenzofuran-5- yl)acetamide | Chiralpak ID, 2*25 cm, 5 um | EtOH in Hex (0.1% FA) | 433 |

Example 159: ¹H NMR (400 MHz, DMSO-d₆) δ 7.92 (s, 2H), 7.62 (s, 1H), 7.60 (s, 1H), 7.08 (dd, J=8.7, 5.5 Hz, 1H), 6.91 (dd, J=11.9, 8.6 Hz, 1H), 5.19 (s, 1H), 3.68 (d, J=2.5 Hz, 2H), 3.20-2.90 (m, 2H), 1.39 (s, 6H), 1.25-1.04 (m, 12H).

Example 183: ¹H NMR (400 MHz, DMSO-d₆) δ 8.07 (br s, 3K), 7.04 (s, 1H), 6.29 (s, 1K), 5.05 (s, 2H), 4.88 (s, 2H), 3.77-3.62 (m, 2H), 3.25-2.90 (m, 2H), 1.48 (s, 6H), 1.15-0.99 (m, 12H).

Example 216: ¹H NMR (300 MHz, DMSO-d₆) δ 7.82 (br s, 2H), 7.59 (s, 1H), 7.58 (s, 1H), 7.03 (s, 1H), 5.19 (s, 1H), 5.04 (s, 2H), 4.87 (s, 2H), 3.67 (s, 2H), 3.25-3.00 (m, 2H), 1.38 (s, 6H), 1.20-0.80 (m, 12H).

The following protocol is suitable for testing the activity of the compounds disclosed herein.

Procedure 1: IL-1β Production in PMA-Differentiated THP-1 Cells Stimulated with Gramicidin.

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3.7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required.

THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1s was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100−0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β3 production in PMA-differentiated THP-1 cells.

Procedure 2: IL-1β Production in PMA-Differentiated THP-1 Cells Stimulated with Gramicidin.

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1p was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 3

1. Experimental procedure
   1.1 Cell Culture
   1) Culture THP-1 cells in the complete RPMI-1640 medium with 10% FBS at 37° C., 5% CO₂.
   2) Passage the cells every 3 days by inoculating 3×10⁻⁵ cells per ml.
   1.2 Compound Preparation
      Prepare the 3-fold serial dilution of the compounds with DMSO in a 384-well LDV Microplate using TECAN EVO system to generate the compound source plate with 10 concentrations. Top concentration is 30 mM.
   1.3 Cell preparation
   1) Centrifuge THP-1 cells at 350 g for 5 min.
   2) Re-suspend cells with complete RMPI-1640 medium, and count cells.
   3) Seed cells in T225 flask, about 2.5×10⁷ per flask, treat cells with 20 ng/ml PMA (final DMSO concentration<1%).
   4) Incubate overnight.
   1.4 THP-1 Stimulation
   1) Wash adherent THP-1 cells with PBS, and detach cells with 4 ml trypsin for T225 flask.
   2) Centrifuge cells at 350 g for 5 min, re-suspend cells with RPMI-1640 containing 2% FBS and count cells with trypan blue.
   3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
   4) Seed 50k cells in 40 ul RPMI-1640 with 2% FBS per well in 384-well plate.
   5) Incubate for 1h at 37° C., 5% CO₂.
   6) Prepare 5× gramicidin, add 10 μl per well, the final concentration is 5 μM, incubate for 2 hrs at 37° C., 5% CO₂.
   7) Centrifuge at 350 g for 1 min.
   8) Pipet 16 μl supernatant by apricot, and transfer into white 384 proxiplate. FIG. 3 depicts the layout of the plates: HC: 100 μM CRID3 (MCC950)+5 μM gramicidin LC: 5 μM Gramicidin.
   1.5 IL-1β detection
   1) Homogenize the 5× diluent #5 with a vortex and add 1 volume of stock solution in 4 volumes of distilled water.
   2) Thaw 20× stock solution of anti-IL1β-Cryptate-antibody and anti-IL1β XL-antibody. Dilute these two antibodies to 1× with detection buffer #3.
   3) Pre-mix the two ready-to-use antibody solutions just prior to use.
   4) Dispense 4 ul of pre-mixed Anti-IL1β antibodies working solution into all wells.
   5) Seal the plate and incubate overnight at 4° C.
   6) Read the cell plate using EnVison and plot Readout vs. the test compound concentration to calculate the IC₅₀.
2. Data Analysis:
1. IC₅₀ of compounds can be calculated using the following formulas

$$Formula for {IC}_{50} \% inhibition = 100 - 100 \times [ {HC}_{ave} - Readout / ( {HC}_{ave} - {LC}_{ave} ) ]$$

2. Fit the normalized data in a dose-response manner using XLfit, and calculate the compound concentration.

Table 12 shows the biological activity of compounds in hTHP-1 assay containing 2% fetal bovine serum: <0.008 μM=“++++++″: 20.008 and <0.04 μM=“+++++”; >0.04 and <0.2 μM=“++++”; ≥0.2 and <1 μM=“++>+”; 1 and <5 μM=“++”; >5 and <30 μM=+”.

TABLE 12
Average IC₅₀ of compounds in hTHP-1 assay

| | Compound | hTHP-1 |
|---|---|---|
| Example # | Number | IC₅₀ |
| 1 | 131 | ++ |
| 2 | 131b or 131a | ++ |
| 3 | 131a or 131b | >30 μM |
| 4 | 129 | + |
| 5 | 129b or 129a | >30 μM |
| 6 | 129a or 129b | ++ |
| 7 | 132 | +++ |
| 8 | 132b | ++ |
| 9 | 132a | +++ |
| 10 | 134 | + |
| 11 | 137 | + |
| 12 | 136 | >30 μM |
| 13 | 133 | +++ |
| 14 | 141 | +++++ |
| 15 | 139 | ++ |
| 16 | 138 | +++ |
| 17 | 140 | +++++ |
| 18 | 142 | ++++ |
| 19 | 143 | +++++ |
| 20 | 197 | +++ |
| 21 | 101 | ++++ |
| 22 | 152 | +++ |
| 23 | 150 | ++ |
| 24 | 148 | +++ |
| 25 | 147 | ++ |
| 26 | 113 | ++ |
| 27 | 151 | ++ |
| 28 | 114 | ++ |
| 29 | 112 | + |
| 30 | 116 | + |
| 31 | 105 | ++ |
| 32 | 117 | + |
| 33 | 109 | +++ |
| 34 | 146 | ++ |
| 35 | 110 | +++ |
| 36 | 126 | +++ |
| 37 | 104 | ++++ |
| 38 | 130 | +++ |
| 39 | 107 | +++ |
| 40 | 106 | +++ |
| 41 | 135 | + |
| 42 | 133b or 133a | ++++ |
| 43 | 133a or 133b | ++ |
| 44 | 141b or 141a | ++++ |
| 45 | 141a or 141b | ++ |
| 46 | 139b or 139a | >30 μM |
| 47 | 139a or 139b | +++ |
| 48 | 138b or 138a | ++++ |
| 49 | 138a or 138b | + |
| 50 | 140b or 140a | +++++ |
| 51 | 140a or 140b | +++ |
| 52 | 144b or 144a | +++++ |
| 53 | 144a or 144b | ++ |
| 54 | 145b or 145a | +++++ |
| 55 | 145a or 145b | ++ |
| 56 | 197b or 197a | +++ |
| 57 | 197a or 197b | ++ |
| 58 | 116b or 116a | >30 μM |
| 59 | 116a or 116b | + |
| 60 | 106b or 106a | ++++ |
| 61 | 106a or 106b | + |
| 62 | 117b or 117a | ++ |
| 63 | 117a or 117b | >30 μM |
| 64 | 152b or 152a | +++ |
| 65 | 152a or 152b | + |
| 66 | 150b or 150a | +++ |
| 67 | 150a or 150b | >30 μM |
| 68 | 148b or 148a | +++ |
| 69 | 148a or 148b | + |
| 70 | 147b or 147a | +++ |
| 71 | 147a or 147b | + |
| 72 | 114b or 114a | ++ |
| 73 | 114a or 114b | >30 μM |
| 74 | 112b or 112a | >30 μM |
| 75 | 112a or 112b | ++ |
| 76 | 101b or 101a | ++++ |
| 77 | 101a or 101b | ++ |
| 78 | 126b or 126a | +++ |
| 79 | 126a or 126b | + |
| 80 | 104b or 104a | +++++ |
| 81 | 104a or 104b | +++ |
| 82 | 130b or 130a | + |
| 83 | 130a or 130b | ++++ |
| 84 | 107b or 107a | ++++ |
| 85 | 107a or 107b | + |
| 86 | 110b or 110a | ++ |
| 87 | 110a or 110b | ++++ |
| 88 | 241 | +++ |
| 89 | 219 | +++ |
| 90 | 218 | + |
| 91 | 217 | + |
| 92 | 214 | >30 μM |
| 93 | 235 | >30 μM |
| 94 | 230 | ++ |
| 95 | 245 | +++ |
| 96 | 244 | >30 μM |
| 97 | 221 | +++ |
| 98 | 220 | + |
| 99 | 304 | ++ |
| 100 | 301 | +++ |
| 101 | 212 | + |
| 102 | 240 | + |
| 103 | 258 | +++ |
| 104 | 257 | +++ |
| 105 | 256 | ++++ |
| 106 | 201 | ++++ |
| 107 | 255 | + |
| 108 | 254 | ++++ |
| 109 | 253 | ++++ |
| 110 | 252 | +++ |
| 111 | 251 | ++++ |
| 112 | 250 | +++ |
| 113 | 249 | +++++ |
| 114 | 248 | ++ |
| 115 | 247 | +++ |
| 116 | 246 | + |
| 117 | 309 | ++++ |
| 118 | 310 | +++ |
| 119 | 243 | +++ |
| 120 | 242 | +++ |
| 121 | 239 | +++ |
| 122 | 238 | +++ |
| 123 | 237 | +++ |
| 124 | 236 | +++ |
| 125 | 234 | +++ |
| 126 | 233 | +++ |
| 127 | 232 | + |
| 128 | 120 | +++ |
| 129 | 229 | ++++ |
| 130 | 228 | ++ |
| 131 | 308 | + |
| 132 | 227 | ++ |
| 133 | 226 | +++ |
| 134 | 225 | ++ |
| 135 | 224 | ++ |
| 136 | 191 | +++ |
| 137 | 222 | +++ |
| 138 | 307 | ++ |
| 139 | 231 | +++ |
| 140 | 306 | +++ |
| 141 | 305 | ++++ |
| 142 | 216 | ++ |
| 143 | 215 | ++ |
| 144 | 203 | + |
| 145 | 213 | +++ |
| 146 | 204 | + |
| 147 | 202 | ++ |
| 148 | 211 | + |
| 149 | 210 | +++ |
| 150 | 209 | + |
| 151 | 208 | +++ |
| 152 | 207 | +++ |
| 153 | 206 | ++ |
| 154 | 205 | + |
| 155 | 261b | +++ |
| 156 | 261a | +++ |
| 157 | 256a | +++++ |
| 158 | 256b | ++ |
| 159 | 201a | ++++ |
| 160 | 201b | +++ |
| 161 | 258a | ++++ |
| 162 | 258b | + |
| 163 | 249a | ++++ |
| 164 | 249b | ++ |
| 165 | 309b | ++ |
| 166 | 309a | ++++ |
| 167 | 239a | ++ |
| 168 | 239b | +++ |
| 169 | 238a | ++ |
| 170 | 238b | +++ |
| 171 | 221a | +++ |
| 172 | 221b | + |
| 173 | 229a | +++ |
| 174 | 229b | + |
| 175 | 234b | ++ |
| 176 | 234a | +++ |
| 177 | 236a | ++++ |
| 178 | 236b | ++ |
| 179 | 242a | +++ |
| 180 | 242b | + |
| 181 | 243a | ++++ |
| 182 | 243b | + |
| 183 | 227a | +++ |
| 184 | 227b | + |
| 185 | 251b | ++++ |
| 186 | 251a | + |
| 187 | 228a | + |
| 188 | 228b | +++ |
| 189 | 226a | ++ |
| 190 | 226b | ++++ |
| 191 | 225a | ++ |
| 192 | 225b | >30 μM |
| 193 | 244b | >30 μM |
| 194 | 244ab | + |
| 195 | 244aa | >30 μM |
| 196 | 220a | + |
| 197 | 220b | >30 μM |
| 198 | 218a | + |
| 199 | 218b | >30 μM |
| 200 | 217a | ++ |
| 201 | 217b | >30 μM |
| 202 | 303 | +++ |
| 203 | 302 | + |
| 204 | 203b | ++ |
| 205 | 203a | >30 μM |
| 206 | 202a | +++ |
| 207 | 202b | >30 μM |
| 208 | 213a | ++++ |
| 209 | 213b | ++ |
| 210 | 210a | + |
| 211 | 210b | +++ |
| 212 | 216a | >30 μM |
| 213 | 216b | ++ |
| 214 | 110a′ | ++++ |
| 215 | 110b′ | ++ |
| 216 | 263a | +++ |
| 217 | 263b | + |
| 218 | 264a | + |
| 219 | 264b | >30 μM |
| 220 | 263 | ++++ |
| 221 | 264 | + |
| | 259 | >17 μM |
| | 259a | + |
| | 259b | >30 μM |
| | 262a | >30 μM |
| | 262b | + |

A number of embodiments of the invention have been described. Nevertheless, it will be understood that various modifications may be made without departing from the spirit and scope of the invention. Accordingly, other embodiments are within the scope of the following claims.

What is claimed is:

1-141. (canceled)

142. A method of treating a disease, disorder or condition that is any one of: a metabolic disorder; a disease of the central nervous system; lung disease; asthma, COPD or pulmonary idiopathic fibrosis; liver disease; pancreatic disease; kidney disease; intestinal disease; skin disease; musculoskeletal disease; vessel disorder; disorder of the bones; eye disease; caused by viral infection; autoimmune disease; cancer or aging;
the method comprising administering to a subject in need of such treatment an effective amount of a compound of Formula AA 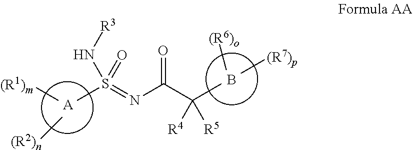
wherein
m=0, 1, or 2;
n=0, 1, or 2;
o=1 or 2;
p=0, 1, 2, or 3;
wherein
A is: thiophenyl optionally substituted with 1 or 2 R¹ and optionally substituted with 1 or 2 R²;
phenyl optionally substituted with 1 or 2 R¹ and optionally substituted with 1 or 2 R²;
thiazolyl optionally substituted with 1 or 2 R¹ and optionally substituted with 1 or 2 R²;
oxazolyl optionally substituted with 1 or 2 R¹ and optionally substituted with 1 or 2 R²;
pyridyl optionally substituted with 1 or 2 R¹ and optionally substituted with 1 or 2 R²;
pyrazolyl optionally substituted with 1 or 2 R¹ and optionally substituted with 1 or 2 R²;
B is phenyl, pyridyl, or pyrimidinyl;
wherein
at least one R⁶ is ortho to the bond connecting the B ring to the C(R⁴R⁵) group of Formula AA;
R¹ and R² are each independently selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,
wherein the C₁-C₆ alkyl and 3- to 7-membered heterocycloalkyl is optionally substituted with one or more substituents each independently selected from hydroxy and oxo;
R⁶ and R¹ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and Ron adjacent atoms, taken together with the atoms connecting them, independently form at least one C₅-C₈ carbocyclic ring, wherein the carbocyclic ring is optionally independently substituted with one or more hydroxy or oxo;
each of R⁴ and R⁵ is independently selected from hydrogen and C₁-C₆ alkyl;
R¹⁰ is C₁-C₆ alkyl;
each of R¹ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R¹ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is hydrogen,
or a pharmaceutically acceptable salt thereof.

143. The method of claim 142, wherein
A is any one of: thiophenyl optionally substituted with 1 or 2 R¹ and optionally substituted with 1 or 2 R²; oxazolyl optionally substituted with 1 or 2 R¹ and optionally substituted with 1 or 2 R²; thiazolyl optionally substituted with 1 or 2 R¹ and optionally substituted with 1 or 2 R²; or phenyl optionally substituted with 1 or 2 R¹ and optionally substituted with 1 or 2 R².

144. The method of claim 142, wherein m=1 and n=0; optionally wherein the substituted ring A is any one of:

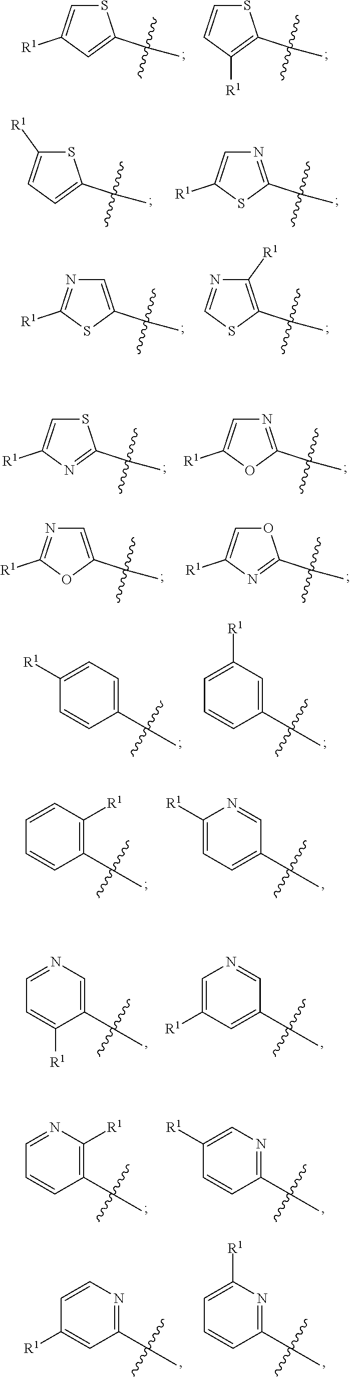

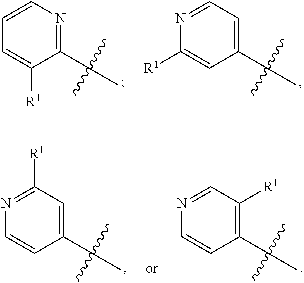

145. The method of claim 142, wherein m=1 and n=1 optionally wherein the substituted ring A is any one of:

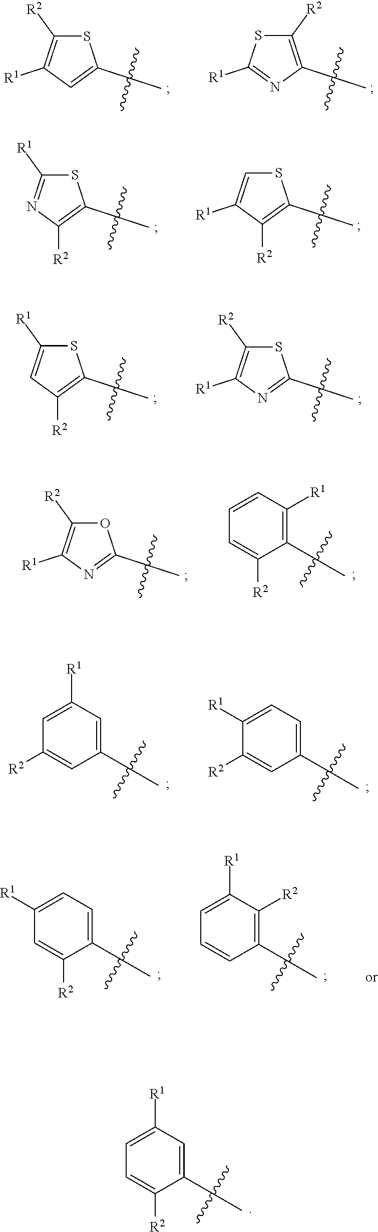

146. The method of claim 142, wherein m=2 and n=1; optionally wherein the substituted ring A is any one of:

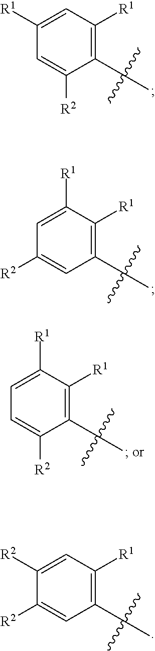

147. The method of claim 142, wherein R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃; and S(O₂)NR¹¹R¹².

148. The method of claim 147, wherein R² is selected from the group consisting of fluoro; chloro; cyano; methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃; and S(O₂)NR¹¹R¹².

149. The method of claim 142, wherein B is phenyl substituted with 1 or 2 R⁶ and optionally substituted with 1, 2, or 3 R⁷; optionally wherein the substituted ring B is any one of:

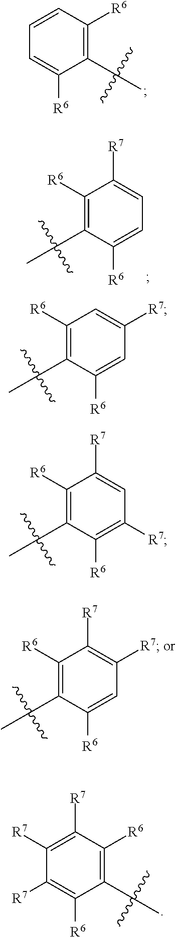

150. The method of claim 142, wherein B is pyridyl; o=1 or 2; and p=0, 1, or 2; optionally wherein the substituted ring B is

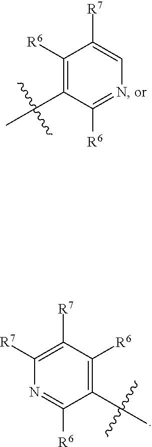

151. The method of claim 142, wherein each of R⁴ and R⁵ is hydrogen.

152. The method of claim 142, wherein the subject is a human.

153. The method of claim 142, wherein the compound of Formula AA is selected from the compounds below: 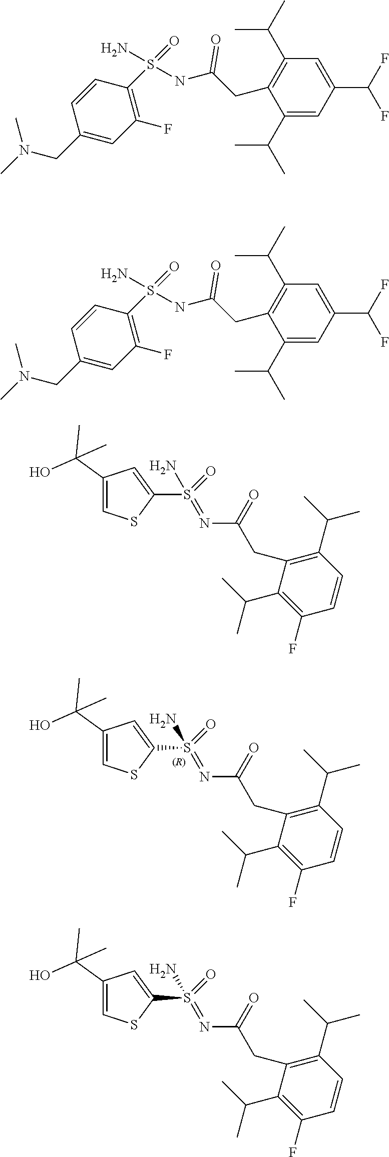 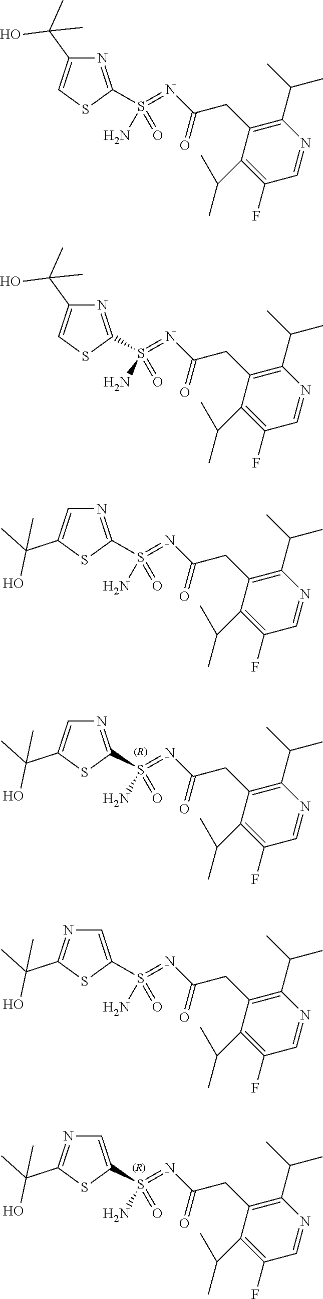 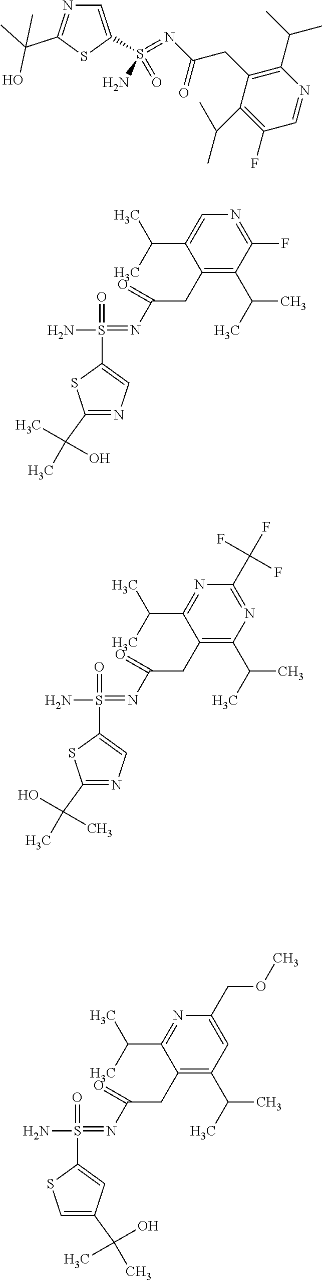 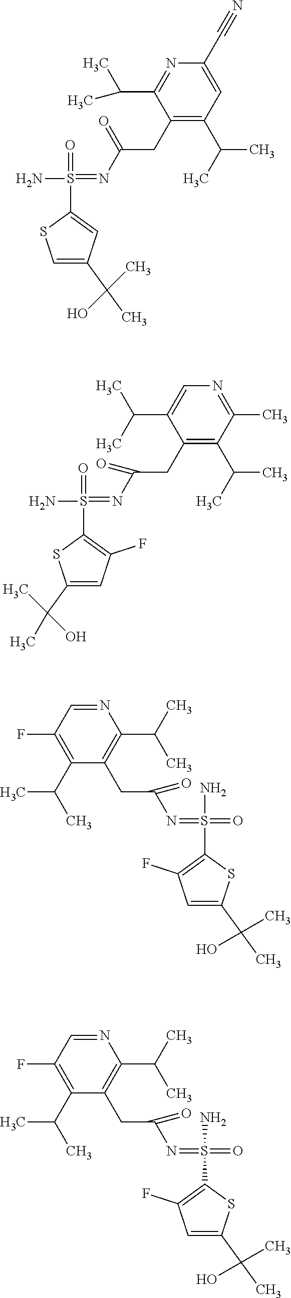 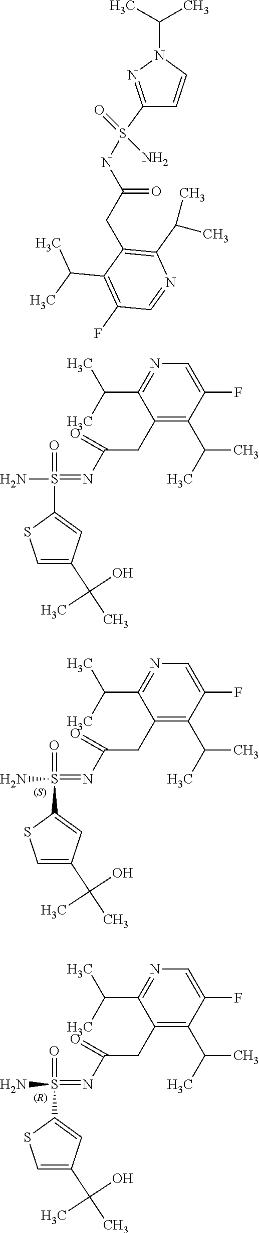 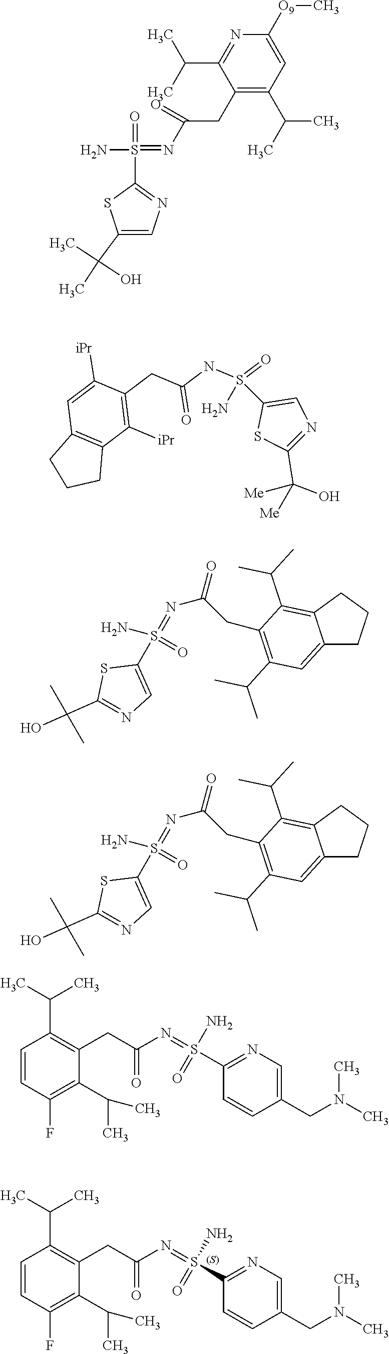 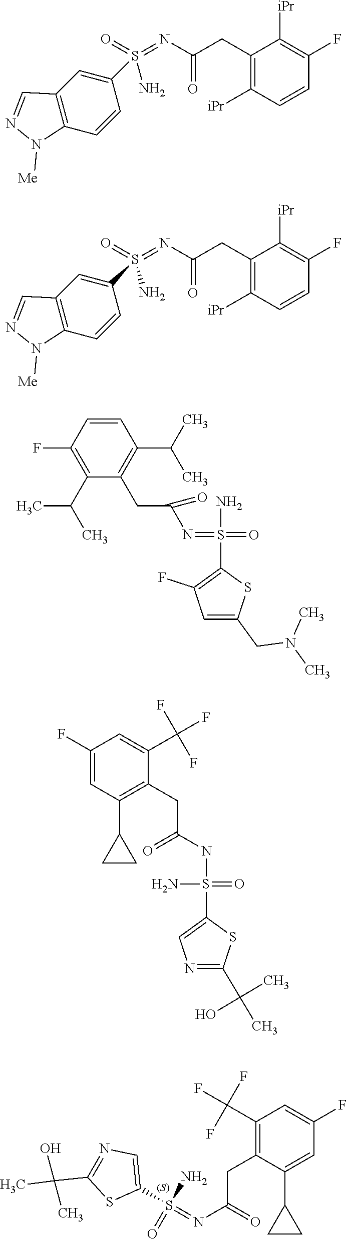 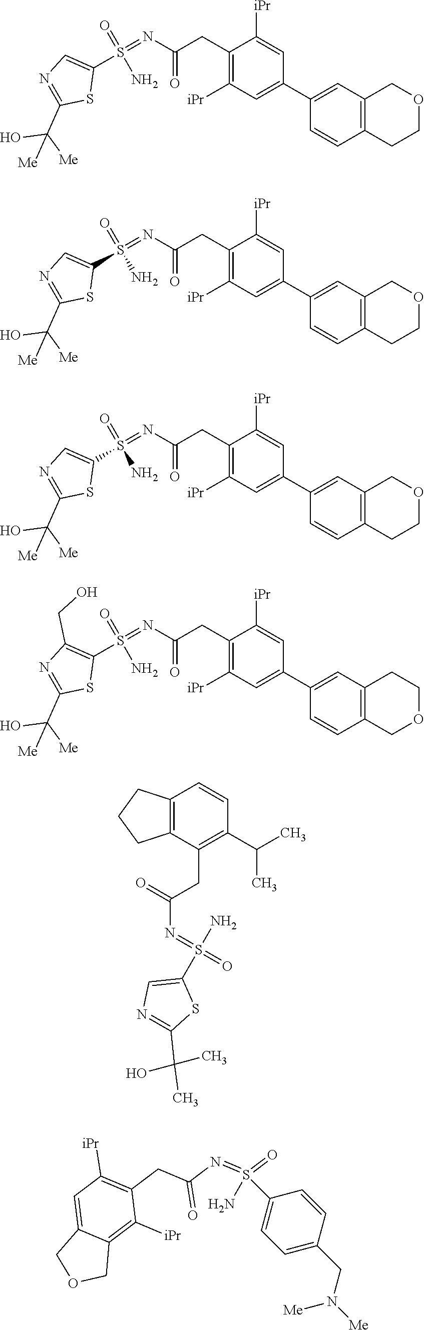 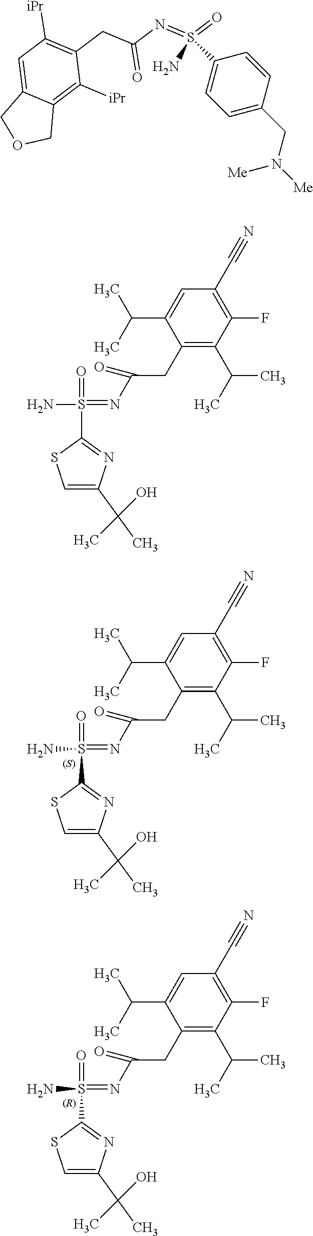 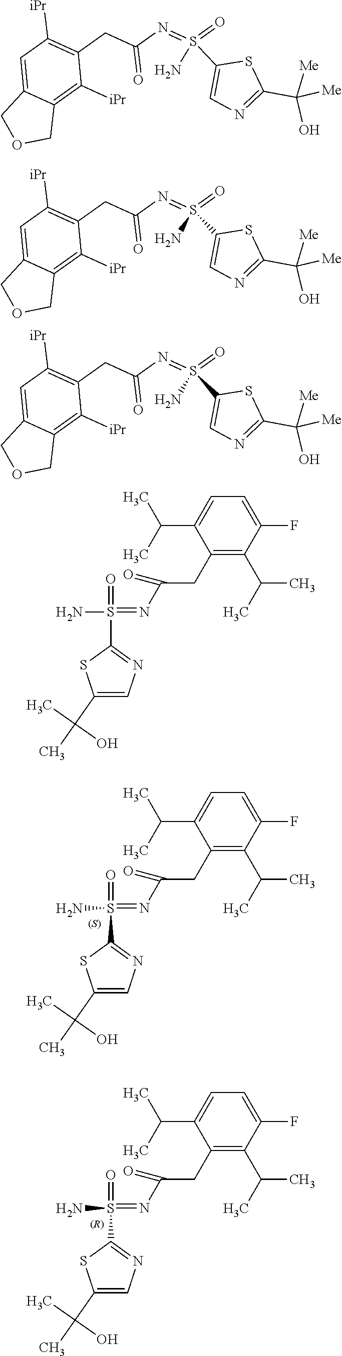 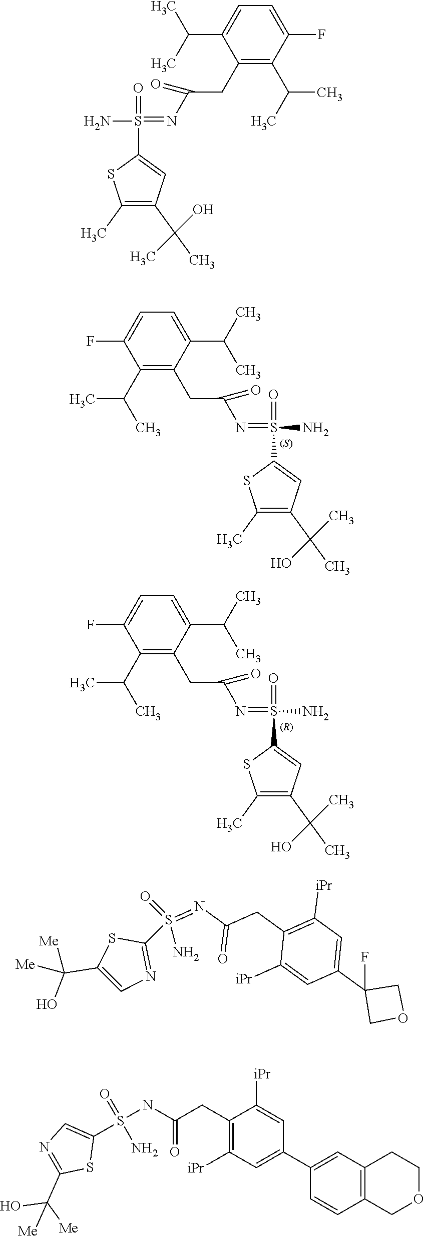 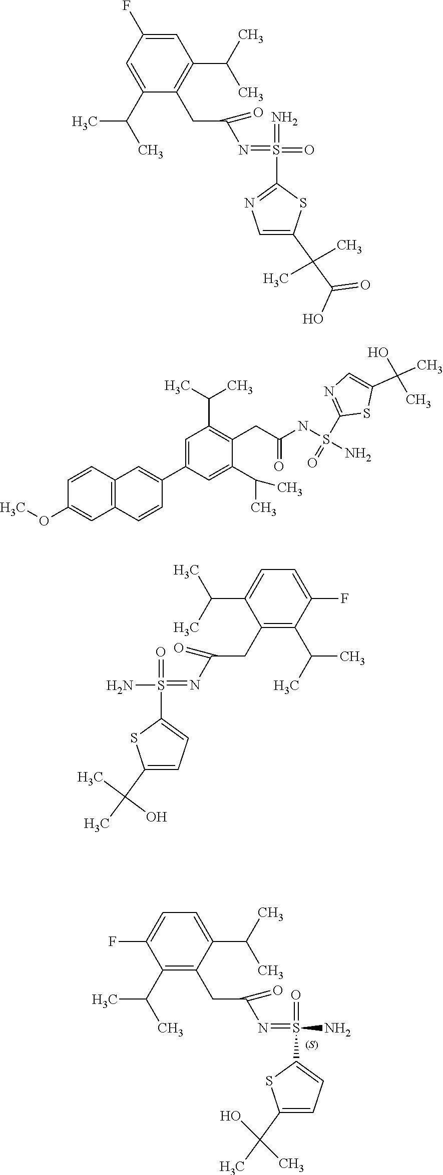 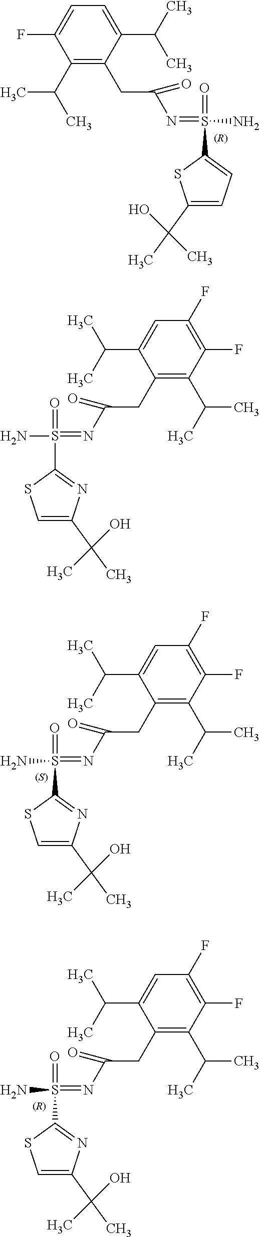 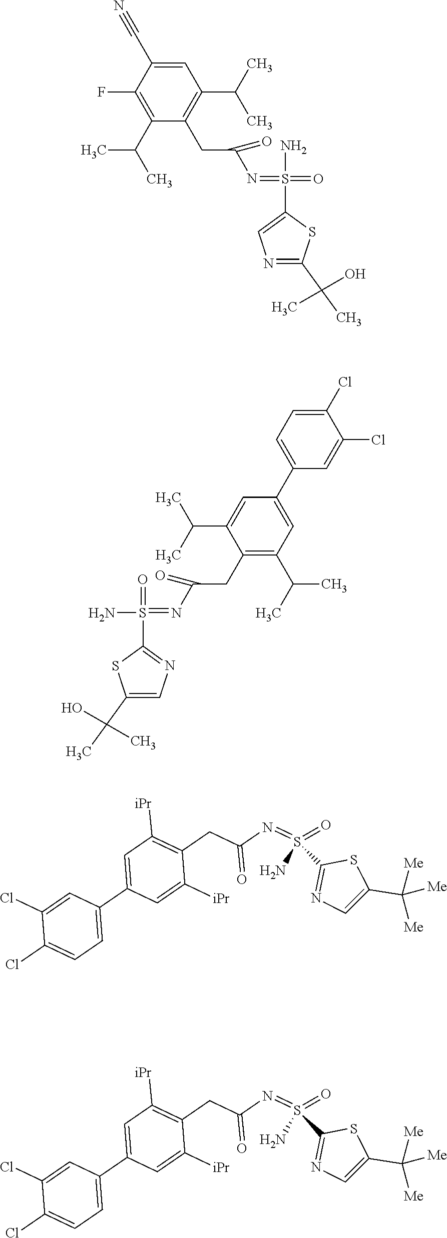 [SMILES: CC(C)c1cc(C#N)c(F)c(C(C)C)c1CC(=O)N=S(N)(=O)c1cnc(C(C)(C)O)s1 ; CC(C)c1cc(-c2ccc(Cl)c(Cl)c2)cc(C(C)C)c1CC(=O)N=S(N)(=O)c1ncc(C(C)(C)O)s1 ; CC(C)c1cc(-c2ccc(Cl)c(Cl)c2)cc(C(C)C)c1CC(=O)N=[S@](N)(=O)c1ncc(C(C)(C)C)s1 ; CC(C)c1cc(-c2ccc(Cl)c(Cl)c2)cc(C(C)C)c1CC(=O)N=[S@@](N)(=O)c1ncc(C(C)(C)C)s1] 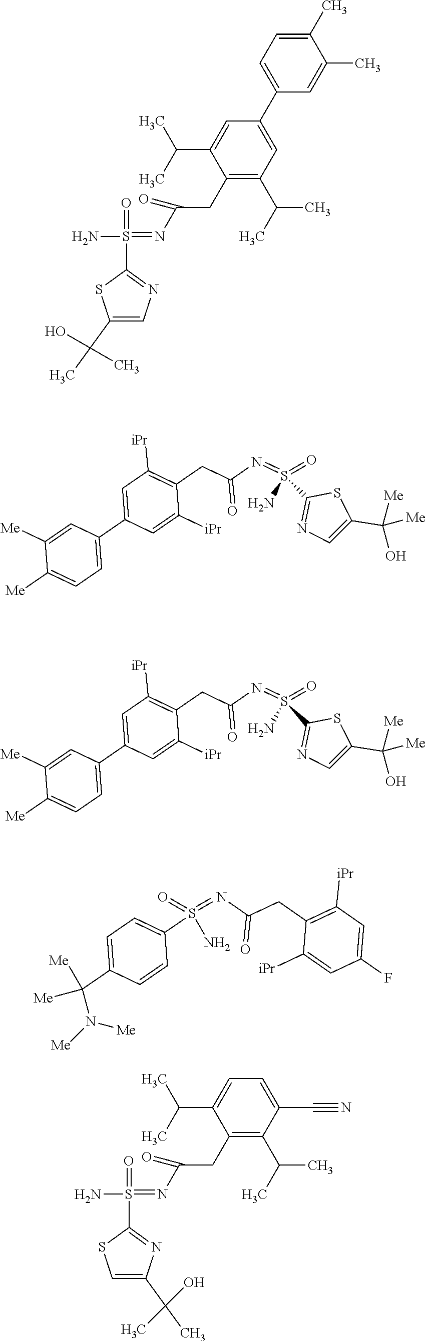 [SMILES: Cc1ccc(-c2cc(C(C)C)c(CC(=O)N=S(N)(=O)c3ncc(C(C)(C)O)s3)c(C(C)C)c2)cc1C ; Cc1ccc(-c2cc(C(C)C)c(CC(=O)N=[S@](N)(=O)c3ncc(C(C)(C)O)s3)c(C(C)C)c2)cc1C ; Cc1ccc(-c2cc(C(C)C)c(CC(=O)N=[S@@](N)(=O)c3ncc(C(C)(C)O)s3)c(C(C)C)c2)cc1C ; CC(C)c1cc(F)cc(C(C)C)c1CC(=O)N=S(N)(=O)c1ccc(C(C)(C)N(C)C)cc1 ; CC(C)c1ccc(C#N)c(C(C)C)c1CC(=O)N=S(N)(=O)c1nc(C(C)(C)O)cs1] 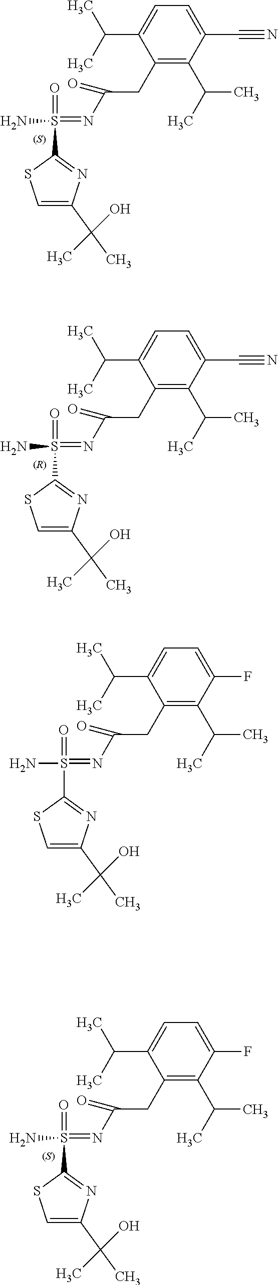 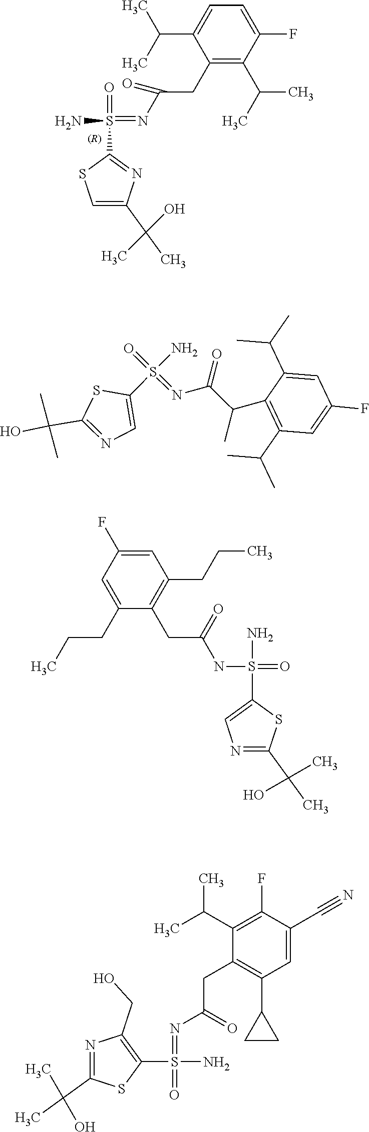 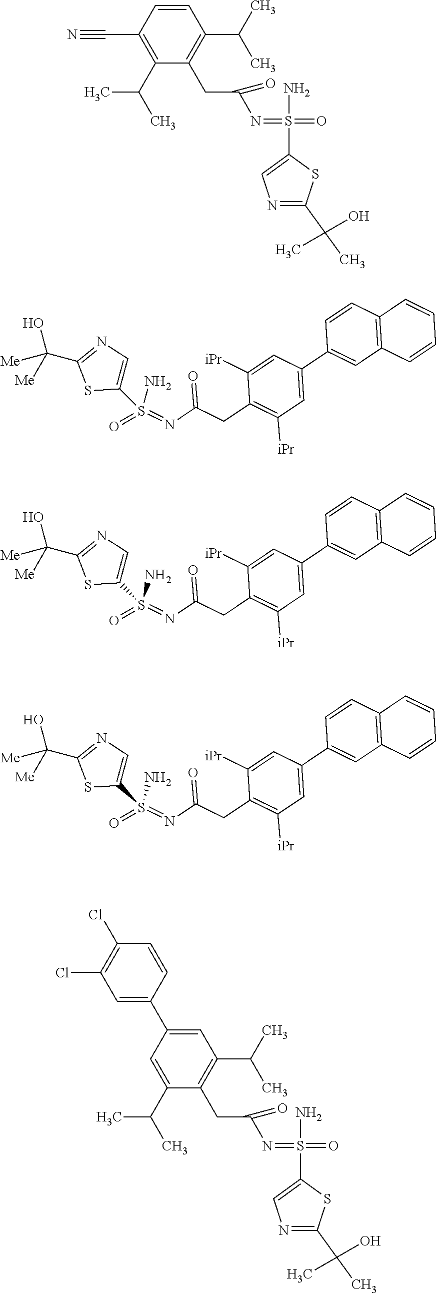 [SMILES: CC(C)c1ccc(C#N)c(C(C)C)c1CC(=O)N=S(N)(=O)c1cnc(C(C)(C)O)s1 ; CC(C)c1cc(-c2ccc3ccccc3c2)cc(C(C)C)c1CC(=O)N=S(N)(=O)c1cnc(C(C)(C)O)s1 ; CC(C)c1cc(-c2ccc3ccccc3c2)cc(C(C)C)c1CC(=O)N=[S@](N)(=O)c1cnc(C(C)(C)O)s1 ; CC(C)c1cc(-c2ccc3ccccc3c2)cc(C(C)C)c1CC(=O)N=[S@@](N)(=O)c1cnc(C(C)(C)O)s1 ; CC(C)c1cc(-c2ccc(Cl)c(Cl)c2)cc(C(C)C)c1CC(=O)N=S(N)(=O)c1cnc(C(C)(C)O)s1] 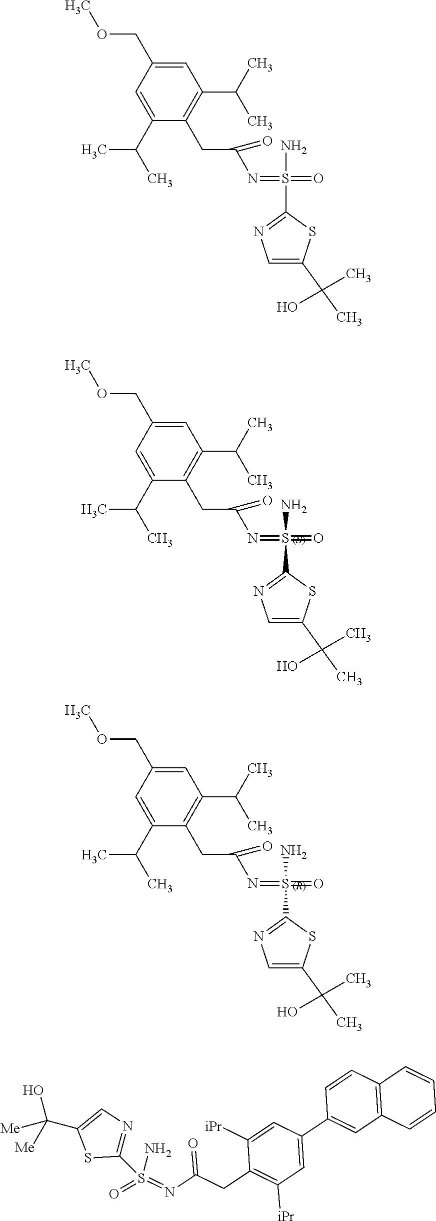 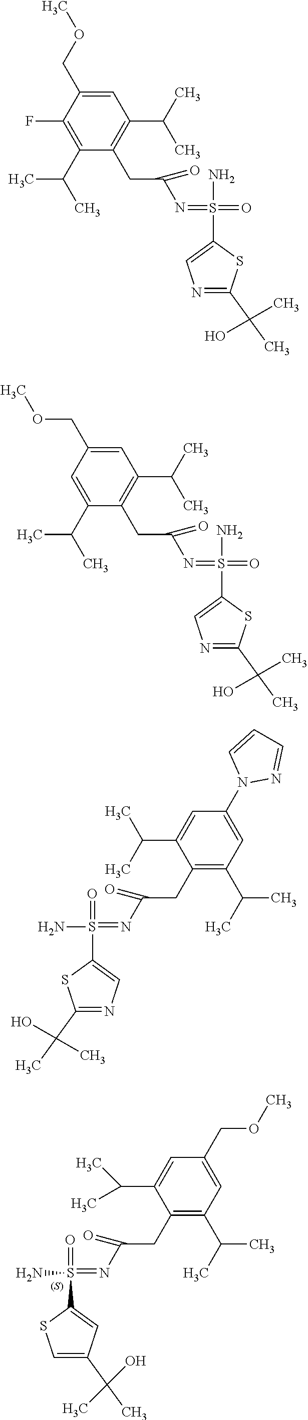 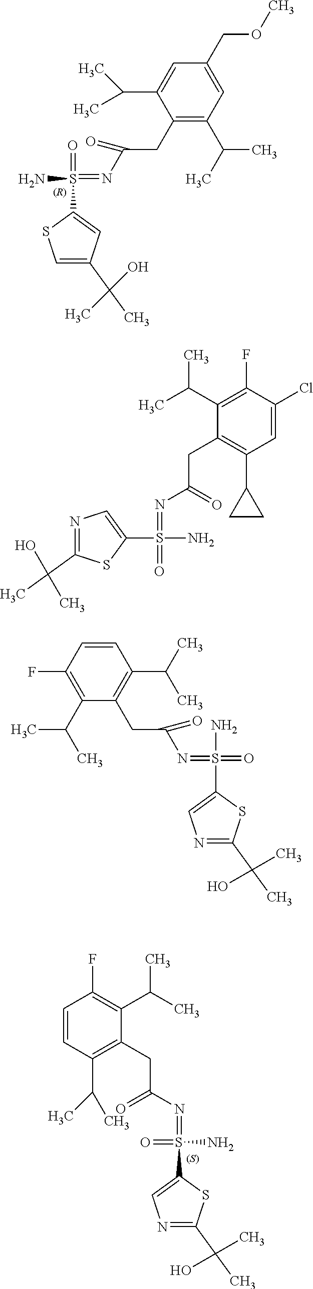 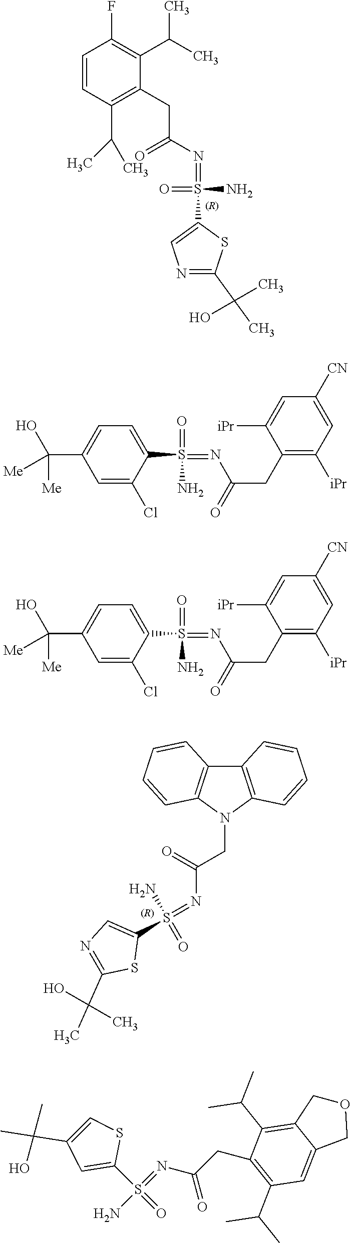 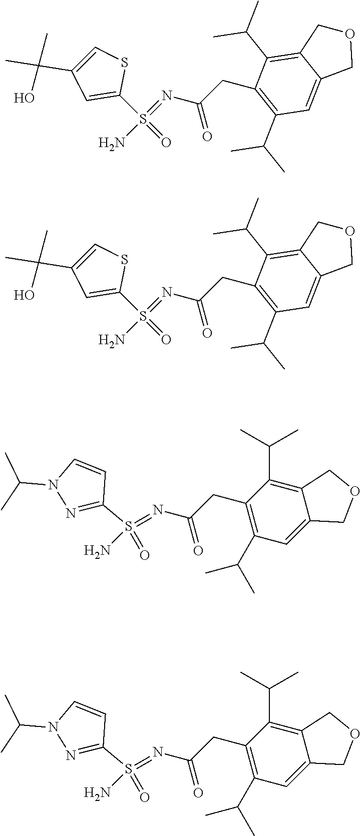
and a pharmaceutically acceptable salt thereof.

154. The method according to claim 142, further comprising administering a therapeutically effective amount of an anti-TNFα agent to the subject;
wherein the compound of Formula AA is administered to the subject prior to administration of the anti-TNFα agent to the subject; or
wherein the anti-TNFα agent is administered to the subject prior to the administration of the compound of Formula AA to the subject; or
wherein the compound of Formula AA and the anti-TNFα agent are administered to the subject at substantially the same time.
